(19) United States
(12) Patent Application Publication
(10) Pub. No.: US 2024/0182561 A1
(43) Pub. Date: Jun. 6, 2024

(54) CALCIUM VOLTAGE-GATED CHANNEL AUXILIARY SUBUNIT GAMMA 1 (CACNG1) BINDING PROTEINS AND CACNG1-MEDIATED DELIVERY TO SKELETAL MUSCLE

(71) Applicant: Regeneron Pharmaceuticals, Inc., Tarrytown, NY (US)

(72) Inventors: Trevor STITT, Crosswicks, NJ (US); Michael Stec, Tarrytown, NY (US); Sandra Kleiner, Yonkers, NY (US); Andrew J. Murphy, Croton-on-Hudson, NY (US); Amy Han, Hockessin, DE (US)

(73) Assignee: Regeneron Pharmaceuticals, Inc., Tarrytown, NY (US)

(21) Appl. No.: 18/501,865

(22) Filed: Nov. 3, 2023

Related U.S. Application Data
(60) Provisional application No. 63/382,418, filed on Nov. 4, 2022.
(60) Provisional application No. 63/422,845, filed on Nov. 4, 2022.
(60) Provisional application No. 63/525,901, filed on Jul. 10, 2023.

(51) Int. Cl. C07K 16/28; A61K 31/568; A61K 31/7105; A61K 47/68; A61P 21/00; C12N 15/113; C12N 15/63

(52) U.S. Cl. CPC C07K 16/28; A61K 31/568; A61K 31/7105; A61K 47/6803; A61P 21/00; C12N 15/113; C12N 15/63; C07K 2317/14; C07K 2317/55; C07K 2317/565; C07K 2317/622; C07K 2317/92; C12N 2310/11; C12N 2310/14; C12N 2310/315

(57) ABSTRACT

The present disclosure provides Calcium Voltage-Gated Channel Auxiliary Subunit Gamma 1 (CACNG1) antigen-binding proteins and protein-drug conjugates including a CACNG1 antigen-binding protein conjugated to a molecular cargo (e.g., a polynucleotide molecule, a polypeptide molecule, a carrier, or a small molecule) for delivery of the molecular cargo to skeletal muscle tissue and/or cells. Methods for treating, preventing, or reducing the likelihood of various skeletal muscle diseases and/or disorders with such antigen-binding proteins or protein-drug conjugates are also provided.

### CROSS REFERENCE TO RELATED APPLICATIONS

This application claims priority to U.S. Provisional Application No. 63/382,418, filed Nov. 4, 2022, U.S. Provisional Application No. 63/422,845, filed Nov. 4, 2022, and U.S. Provisional Application No. 63/525,901, filed Jul. 10, 2023, the disclosure of each of which is herein incorporated by reference in its entirety.

### SEQUENCE LISTING

The instant application contains a Sequence Listing which has been submitted electronically in XML file format and is hereby incorporated by reference in its entirety. Said XML copy, created on Oct. 31, 2023, is named 250298_000561_SL.xml and is 522,807 bytes in size.

### FIELD OF THE INVENTION

The present disclosure relates to Calcium Voltage-Gated Channel Auxiliary Subunit Gamma 1 (CACNG1) antigen-binding proteins and protein-drug conjugates, including a CACNG1 antigen-binding protein conjugated to a molecular cargo, as well as methods of treating, preventing, or reducing the likelihood of diseases with such protein-drug conjugates.

### BACKGROUND OF THE INVENTION

Recent advances in the field of genetic medicine have led to the development of therapies that have the potential to treat several muscle diseases. However, the delivery of these therapies to muscle remains a major challenge, as skeletal muscle is a large tissue, comprising ˜40% of the total body mass. Targeting therapies to muscle is typically achieved via high dose, systemic delivery, which often results in uptake in other organs and adverse events (e.g., liver toxicity), along with inefficient muscle uptake. Accordingly, there remains a need to develop skeletal muscle-specific therapies with efficient muscle uptake that can disrupt the onset and/or the course of muscle diseases (e.g., facioscapulohumeral muscular dystrophy, myotonic dystrophy, Duchenne muscular dystrophy), in particular, in order to improve the quality of the lives of those suffering from such diseases.

### SUMMARY OF THE INVENTION

The present disclosure provides Calcium Voltage-Gated Channel Auxiliary Subunit Gamma 1 (CACNG1) antigen-binding proteins and protein-drug conjugates including a CACNG1 antigen-binding protein conjugated to a molecular cargo (e.g., a polynucleotide molecule, a polypeptide molecule, a carrier, or a small molecule) for delivery of the molecular cargo to skeletal muscle tissue and/or cells. The antigen-binding protein can comprise a heavy chain variable region (HCVR or V_{H}) and a light chain variable region (LCVR or V_{L}). Methods for treating, preventing, or reducing the likelihood of various skeletal muscle diseases and/or disorders with such antigen-binding proteins or protein-drug conjugates are also provided.

In one aspect, provided herein is an antigen-binding protein that binds specifically Calcium Voltage-Gated Channel Auxiliary Subunit Gamma 1 (CACNG1), comprising:
(i) an HCVR that comprises the HCDR1, HCDR2, and HCDR3 of an HCVR comprising the amino acid sequence set forth in SEQ ID NO: 1, 9, 17, 25, 33, 41, 49, 57, 65, 73, 81, 89, 97, 105, 113, 121, 129, 137, 429, or 451 (or a variant thereof);
and/or
(ii) an LCVR that comprises the LCDR1, LCDR2, and LCDR3 of an LCVR comprising the amino acid sequence set forth in SEQ ID NO: 5, 13, 21, 29, 37, 45, 53, 61, 69, 77, 85, 93,101, 109, 117, 125, 133, 141, 437, or 459 (or a variant thereof).

In some embodiments of the above-described antigen-binding protein, the antigen-binding protein comprises:
(1) an HCVR comprising the HCDR1, HCDR2, and HCDR3 of an HCVR that comprises the amino acid sequence set forth in SEQ ID NO: 1 (or a variant thereof); and an LCVR comprising the LCDR1, LCDR2, and LCDR3 of an LCVR that comprises the amino acid sequence set forth in SEQ ID NO: 5 (or a variant thereof);
(2) an HCVR comprising the HCDR1, HCDR2, and HCDR3 of an HCVR that comprises the amino acid sequence set forth in SEQ ID NO: 9; and an LCVR comprising the LCDR1, LCDR2, and LCDR3 of an LCVR that comprises the amino acid sequence set forth in SEQ ID NO: 13 (or a variant thereof);
(3) an HCVR comprising the HCDR1, HCDR2, and HCDR3 of an HCVR that comprises the amino acid sequence set forth in SEQ ID NO: 17; and an LCVR comprising the LCDR1, LCDR2, and LCDR3 of an LCVR that comprises the amino acid sequence set forth in SEQ ID NO: 21 (or a variant thereof);
(4) an HCVR comprising the HCDR1, HCDR2, and HCDR3 of an HCVR that comprises the amino acid sequence set forth in SEQ ID NO: 25 (or a variant thereof); and an LCVR comprising the LCDR1, LCDR2, and LCDR3 of an LCVR that comprises the amino acid sequence set forth in SEQ ID NO: 29 (or a variant thereof);
(5) an HCVR comprising the HCDR1, HCDR2, and HCDR3 of an HCVR that comprises the amino acid sequence set forth in SEQ ID NO: 33 (or a variant thereof); and an LCVR comprising the LCDR1, LCDR2, and LCDR3 of an LCVR that comprises the amino acid sequence set forth in SEQ ID NO: 37 (or a variant thereof);
(6) an HCVR comprising the HCDR1, HCDR2, and HCDR3 of an HCVR that comprises the amino acid sequence set forth in SEQ ID NO: 41 (or a variant thereof); and an LCVR comprising the LCDR1, LCDR2, and LCDR3 of an LCVR that comprises the amino acid sequence set forth in SEQ ID NO: 45 (or a variant thereof);
(7) an HCVR comprising the HCDR1, HCDR2, and HCDR3 of an HCVR that comprises the amino acid sequence set forth in SEQ ID NO: 49 (or a variant thereof); and an LCVR comprising the LCDR1, LCDR2, and LCDR3 of an LCVR that comprises the amino acid sequence set forth in SEQ ID NO: 53 (or a variant thereof);
(8) an HCVR comprising the HCDR1, HCDR2, and HCDR3 of an HCVR that comprises the amino acid sequence set forth in SEQ ID NO: 57 (or a variant thereof); and an LCVR comprising the LCDR1, LCDR2, and LCDR3 of an LCVR that comprises the amino acid sequence set forth in SEQ ID NO: 61 (or a variant thereof);
(9) an HCVR comprising the HCDR1, HCDR2, and HCDR3 of an HCVR that comprises the amino acid sequence set forth in SEQ ID NO: 65 (or a variant thereof); and an LCVR comprising the LCDR1, LCDR2, and LCDR3 of an LCVR that comprises the amino acid sequence set forth in SEQ ID NO: 69 (or a variant thereof);
(10) an HCVR comprising the HCDR1, HCDR2, and HCDR3 of an HCVR that comprises the amino acid sequence set forth in SEQ ID NO: 73 (or a variant thereof); and an LCVR comprising the LCDR1, LCDR2, and LCDR3 of an LCVR that comprises the amino acid sequence set forth in SEQ ID NO: 77 (or a variant thereof);
(11) an HCVR comprising the HCDR1, HCDR2, and HCDR3 of an HCVR that comprises the amino acid sequence set forth in SEQ ID NO: 81 (or a variant thereof); and an LCVR comprising the LCDR1, LCDR2, and LCDR3 of an LCVR that comprises the amino acid sequence set forth in SEQ ID NO: 85 (or a variant thereof);
(12) an HCVR comprising the HCDR1, HCDR2, and HCDR3 of an HCVR that comprises the amino acid sequence set forth in SEQ ID NO: 89 (or a variant thereof); and an LCVR comprising the LCDR1, LCDR2, and LCDR3 of an LCVR that comprises the amino acid sequence set forth in SEQ ID NO: 93 (or a variant thereof);
(13) an HCVR comprising the HCDR1, HCDR2, and HCDR3 of an HCVR that comprises the amino acid sequence set forth in SEQ ID NO: 97 (or a variant thereof); and an LCVR comprising the LCDR1, LCDR2, and LCDR3 of an LCVR that comprises the amino acid sequence set forth in SEQ ID NO: 101 (or a variant thereof);
(14) an HCVR comprising the HCDR1, HCDR2, and HCDR3 of an HCVR that comprises the amino acid sequence set forth in SEQ ID NO: 105 (or a variant thereof); and an LCVR comprising the LCDR1, LCDR2, and LCDR3 of an LCVR that comprises the amino acid sequence set forth in SEQ ID NO: 109 (or a variant thereof);
(15) an HCVR comprising the HCDR1, HCDR2, and HCDR3 of an HCVR that comprises the amino acid sequence set forth in SEQ ID NO: 113 (or a variant thereof); and an LCVR comprising the LCDR1, LCDR2, and LCDR3 of an LCVR that comprises the amino acid sequence set forth in SEQ ID NO: 117 (or a variant thereof);
(16) an HCVR comprising the HCDR1, HCDR2, and HCDR3 of an HCVR that comprises the amino acid sequence set forth in SEQ ID NO: 121 (or a variant thereof); and an LCVR comprising the LCDR1, LCDR2, and LCDR3 of an LCVR that comprises the amino acid sequence set forth in SEQ ID NO: 125 (or a variant thereof);
(17) an HCVR comprising the HCDR1, HCDR2, and HCDR3 of an HCVR that comprises the amino acid sequence set forth in SEQ ID NO: 129 (or a variant thereof); and an LCVR comprising the LCDR1, LCDR2, and LCDR3 of an LCVR that comprises the amino acid sequence set forth in SEQ ID NO: 133 (or a variant thereof);
(18) an HCVR comprising the HCDR1, HCDR2, and HCDR3 of an HCVR that comprises the amino acid sequence set forth in SEQ ID NO: 137 (or a variant thereof); and an LCVR comprising the LCDR1, LCDR2, and LCDR3 of an LCVR that comprises the amino acid sequence set forth in SEQ ID NO: 141 (or a variant thereof);
(19) an HCVR comprising the HCDR1, HCDR2, and HCDR3 of an HCVR that comprises the amino acid sequence set forth in SEQ ID NO: 429 (or a variant thereof); and an LCVR comprising the LCDR1, LCDR2, and LCDR3 of an LCVR that comprises the amino acid sequence set forth in SEQ ID NO: 437 (or a variant thereof); or
(20) an HCVR comprising the HCDR1, HCDR2, and HCDR3 of an HCVR that comprises the amino acid sequence set forth in SEQ ID NO: 451 (or a variant thereof); and an LCVR comprising the LCDR1, LCDR2, and LCDR3 of an LCVR that comprises the amino acid sequence set forth in SEQ ID NO: 459 (or a variant thereof).

In some embodiments of the above-described antigen-binding protein, the antigen-binding protein comprises:
(a) an HCVR that comprises: an HCDR1 comprising the amino acid sequence set forth in SEQ ID NO: 2 (or a variant thereof), an HCDR2 comprising the amino acid sequence set forth in SEQ ID NO: 3 (or a variant thereof), and an HCDR3 comprising the amino acid sequence set forth in SEQ ID NO: 4 (or a variant thereof); and an LCVR that comprises: an LCDR1 comprising the amino acid sequence set forth in SEQ ID NO: 6 (or a variant thereof), an LCDR2 comprising the amino acid sequence set forth in SEQ ID NO: 7 (or a variant thereof), and an LCDR3 comprising the amino acid sequence set forth in SEQ ID NO: 8 (or a variant thereof);
(b) an HCVR that comprises: an HCDR1 comprising the amino acid sequence set forth in SEQ ID NO: 10 (or a variant thereof), an HCDR2 comprising the amino acid sequence set forth in SEQ ID NO: 11 (or a variant thereof), and an HCDR3 comprising the amino acid sequence set forth in SEQ ID NO: 12 (or a variant thereof); and an LCVR that comprises: an LCDR1 comprising the amino acid sequence set forth in SEQ ID NO: 14 (or a variant thereof), an LCDR2 comprising the amino acid sequence set forth in SEQ ID NO: 15 (or a variant thereof), and an LCDR3 comprising the amino acid sequence set forth in SEQ ID NO: 16 (or a variant thereof);
(c) an HCVR that comprises: an HCDR1 comprising the amino acid sequence set forth in SEQ ID NO: 18 (or a variant thereof), an HCDR2 comprising the amino acid sequence set forth in SEQ ID NO: 19 (or a variant thereof), and an HCDR3 comprising the amino acid sequence set forth in SEQ ID NO: 20 (or a variant thereof); and an LCVR that comprises: an LCDR1 comprising the amino acid sequence set forth in SEQ ID NO: 22 (or a variant thereof), an LCDR2 comprising the amino acid sequence set forth in SEQ ID NO: 23 (or a variant thereof), and an LCDR3 comprising the amino acid sequence set forth in SEQ ID NO: 24 (or a variant thereof);
(d) an HCVR that comprises: an HCDR1 comprising the amino acid sequence set forth in SEQ ID NO: 26 (or a variant thereof), an HCDR2 comprising the amino acid sequence set forth in SEQ ID NO: 27 (or a variant thereof), and an HCDR3 comprising the amino acid sequence set forth in SEQ ID NO: 28 (or a variant thereof); and an LCVR that comprises: an LCDR1 comprising the amino acid sequence set forth in SEQ ID NO: 30 (or a variant thereof), an LCDR2 comprising the amino acid sequence set forth in SEQ ID NO: 31 (or a variant thereof), and an LCDR3 comprising the amino acid sequence set forth in SEQ ID NO: 32 (or a variant thereof);
(e) an HCVR that comprises: an HCDR1 comprising the amino acid sequence set forth in SEQ ID NO: 34 (or a variant thereof), an HCDR2 comprising the amino acid sequence set forth in SEQ ID NO: 35 (or a variant thereof), and an HCDR3 comprising the amino acid sequence set forth in SEQ ID NO: 36 (or a variant thereof); and an LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof);
(f) an HCVR that comprises: an HCDR1 comprising the amino acid sequence set forth in SEQ ID NO: 42 (or a variant thereof), an HCDR2 comprising the amino acid sequence set forth in SEQ ID NO: 43 (or a variant thereof), and an HCDR3 comprising the amino acid sequence set forth in SEQ ID NO: 44 (or a variant thereof); and
an LCVR that comprises: an LCDR1 comprising the amino acid sequence set forth in SEQ ID NO: 46 (or a variant thereof), an LCDR2 comprising the amino acid sequence set forth in SEQ ID NO: 47 (or a variant thereof), and an LCDR3 comprising the amino acid sequence set forth in SEQ ID NO: 48 (or a variant thereof);
(g) an HCVR that comprises: an HCDR1 comprising the amino acid sequence set forth in SEQ ID NO: 50 (or a variant thereof), an HCDR2 comprising the amino acid sequence set forth in SEQ ID NO: 51 (or a variant thereof), and an HCDR3 comprising the amino acid sequence set forth in SEQ ID NO: 52 (or a variant thereof); and an LCVR that comprises: an LCDR1 comprising the amino acid sequence set forth in SEQ ID NO: 54 (or a variant thereof), an LCDR2 comprising the amino acid sequence set forth in SEQ ID NO: 55 (or a variant thereof), and an LCDR3 comprising the amino acid sequence set forth in SEQ ID NO: 56 (or a variant thereof);
(h) an HCVR that comprises: an HCDR1 comprising the amino acid sequence set forth in SEQ ID NO: 58 (or a variant thereof), an HCDR2 comprising the amino acid sequence set forth in SEQ ID NO: 59 (or a variant thereof), and an HCDR3 comprising the amino acid sequence set forth in SEQ ID NO: 60 (or a variant thereof); and an LCVR that comprises: an LCDR1 comprising the amino acid sequence set forth in SEQ ID NO: 62 (or a variant thereof), an LCDR2 comprising the amino acid sequence set forth in SEQ ID NO: 63 (or a variant thereof), and an LCDR3 comprising the amino acid sequence set forth in SEQ ID NO: 64 (or a variant thereof);
(i) an HCVR that comprises: an HCDR1 comprising the amino acid sequence set forth in SEQ ID NO: 66 (or a variant thereof), an HCDR2 comprising the amino acid sequence set forth in SEQ ID NO: 67 (or a variant thereof), and an HCDR3 comprising the amino acid sequence set forth in SEQ ID NO: 68 (or a variant thereof); and an LCVR that comprises: an LCDR1 comprising the amino acid sequence set forth in SEQ ID NO: 70 (or a variant thereof), an LCDR2 comprising the amino acid sequence set forth in SEQ ID NO: 71 (or a variant thereof), and an LCDR3 comprising the amino acid sequence set forth in SEQ ID NO: 72 (or a variant thereof);
(j) an HCVR that comprises: an HCDR1 comprising the amino acid sequence set forth in SEQ ID NO: 74 (or a variant thereof), an HCDR2 comprising the amino acid sequence set forth in SEQ ID NO: 75 (or a variant thereof), and an HCDR3 comprising the amino acid sequence set forth in SEQ ID NO: 76 (or a variant thereof); and an LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof);
(k) an HCVR that comprises: an HCDR1 comprising the amino acid sequence set forth in SEQ ID NO: 82 (or a variant thereof), an HCDR2 comprising the amino acid sequence set forth in SEQ ID NO: 83 (or a variant thereof), and an HCDR3 comprising the amino acid sequence set forth in SEQ ID NO: 84 (or a variant thereof); and an LCVR that comprises: an LCDR1 comprising the amino acid sequence set forth in SEQ ID NO: 86 (or a variant thereof), an LCDR2 comprising the amino acid sequence set forth in SEQ ID NO: 87 (or a variant thereof), and an LCDR3 comprising the amino acid sequence set forth in SEQ ID NO: 88 (or a variant thereof);
(l) an HCVR that comprises: an HCDR1 comprising the amino acid sequence set forth in SEQ ID NO: 90 (or a variant thereof), an HCDR2 comprising the amino acid sequence set forth in SEQ ID NO: 91 (or a variant thereof), and an HCDR3 comprising the amino acid sequence set forth in SEQ ID NO: 92 (or a variant thereof); and an LCVR that comprises: an LCDR1 comprising the amino acid sequence set forth in SEQ ID NO: 94 (or a variant thereof), an LCDR2 comprising the amino acid sequence set forth in SEQ ID NO: 95 (or a variant thereof), and an LCDR3 comprising the amino acid sequence set forth in SEQ ID NO: 96 (or a variant thereof);
(m) an HCVR that comprises: an HCDR1 comprising the amino acid sequence set forth in SEQ ID NO: 98 (or a variant thereof), an HCDR2 comprising the amino acid sequence set forth in SEQ ID NO: 99 (or a variant thereof), and an HCDR3 comprising the amino acid sequence set forth in SEQ ID NO: 100 (or a variant thereof); and an LCVR that comprises: an LCDR1 comprising the amino acid sequence set forth in SEQ ID NO: 102 (or a variant thereof), an LCDR2 comprising the amino acid sequence set forth in SEQ ID NO: 103 (or a variant thereof), and an LCDR3 comprising the amino acid sequence set forth in SEQ ID NO: 104 (or a variant thereof);
(n) an HCVR that comprises: an HCDR1 comprising the amino acid sequence set forth in SEQ ID NO: 106 (or a variant thereof), an HCDR2 comprising the amino acid sequence set forth in SEQ ID NO: 107 (or a variant thereof), and an HCDR3 comprising the amino acid sequence set forth in SEQ ID NO: 108 (or a variant thereof); and an LCVR that comprises: an LCDR1 comprising the amino acid sequence set forth in SEQ ID NO: 110 (or a variant thereof), an LCDR2 comprising the amino acid sequence set forth in SEQ ID NO: 111 (or a variant thereof), and an LCDR3 comprising the amino acid sequence set forth in SEQ ID NO: 112 (or a variant thereof);
(o) an HCVR that comprises: an HCDR1 comprising the amino acid sequence set forth in SEQ ID NO: 114 (or a variant thereof), an HCDR2 comprising the amino acid sequence set forth in SEQ ID NO: 115 (or a variant thereof), and an HCDR3 comprising the amino acid sequence set forth in SEQ ID NO: 116 (or a variant thereof); and an LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 102 (or a variant thereof);
(p) an HCVR that comprises: an HCDR1 comprising the amino acid sequence set forth in SEQ ID NO: 122 (or a variant thereof), an HCDR2 comprising the amino acid sequence set forth in SEQ ID NO: 123 (or a variant thereof), and an HCDR3 comprising the amino acid sequence set forth in SEQ ID NO: 124 (or a variant thereof); and an LCVR that comprises: an LCDR1 comprising the amino acid sequence set forth in SEQ ID NO: 126 (or a variant thereof), an LCDR2 comprising the amino acid sequence set forth in SEQ ID NO: 127 (or a variant thereof), and an LCDR3 comprising the amino acid sequence set forth in SEQ ID NO: 128 (or a variant thereof);
(q) an HCVR that comprises: an HCDR1 comprising the amino acid sequence set forth in SEQ ID NO: 130 (or a variant thereof), an HCDR2 comprising the amino acid sequence set forth in SEQ ID NO: 131 (or a variant thereof), and an HCDR3 comprising the amino acid sequence set forth in SEQ ID NO: 132 (or a variant thereof); and an LCVR that comprises: an LCDR1 comprising the amino acid sequence set forth in SEQ ID NO: 134 (or a variant thereof), an LCDR2 comprising the amino acid sequence set forth in SEQ ID NO: 135 (or a variant thereof), and an LCDR3 comprising the amino acid sequence set forth in SEQ ID NO: 136 (or a variant thereof);
(r) an HCVR that comprises: an HCDR1 comprising the amino acid sequence set forth in SEQ ID NO: 138 (or a variant thereof), an HCDR2 comprising the amino acid sequence set forth in SEQ ID NO: 139 (or a variant thereof), and an HCDR3 comprising the amino acid sequence set forth in SEQ ID NO: 140 (or a variant thereof); and an LCVR that comprises: an LCDR1 comprising the amino acid sequence set forth in SEQ ID NO: 142 (or a variant thereof), an LCDR2 comprising the amino acid sequence set forth in SEQ ID NO: 143 (or a variant thereof), and an LCDR3 comprising the amino acid sequence set forth in SEQ ID NO: 144 (or a variant thereof);
(s) an HCVR that comprises: an HCDR1 comprising the amino acid sequence set forth in SEQ ID NO: 431 (or a variant thereof), an HCDR2 comprising the amino acid sequence set forth in SEQ ID NO: 433 (or a variant thereof), and an HCDR3 comprising the amino acid sequence set forth in SEQ ID NO: 435 (or a variant thereof); and an LCVR that comprises: an LCDR1 comprising the amino acid sequence set forth in SEQ ID NO: 439 (or a variant thereof), an LCDR2 comprising the amino acid sequence YNS (or a variant thereof), and an LCDR3 comprising the amino acid sequence set forth in SEQ ID NO: 441 (or a variant thereof); or
(t) an HCVR that comprises: an HCDR1 comprising the amino acid sequence set forth in SEQ ID NO: 453 (or a variant thereof), an HCDR2 comprising the amino acid sequence set forth in SEQ ID NO: 455 (or a variant thereof), and an HCDR3 comprising the amino acid sequence set forth in SEQ ID NO: 457 (or a variant thereof); and an LCVR that comprises: an LCDR1 comprising the amino acid sequence set forth in SEQ ID NO: 461 (or a variant thereof), an LCDR2 comprising the amino acid sequence RNN (or a variant thereof), and an LCDR3 comprising the amino acid sequence set forth in SEQ ID NO: 463 (or a variant thereof).

In some embodiments of any of the above-described antigen-binding proteins, the antigen-binding protein comprises:
(1) an HCVR that comprises the amino acid sequence set forth in SEQ ID NO: 1 (or a variant thereof); and an LCVR that comprises the amino acid sequence set forth in SEQ ID NO: 5 (or a variant thereof);
(2) an HCVR that comprises the amino acid sequence set forth in SEQ ID NO: 9; and an LCVR that comprises the amino acid sequence set forth in SEQ ID NO: 13 (or a variant thereof);
(3) an HCVR that comprises the amino acid sequence set forth in SEQ ID NO: 17; and an LCVR that comprises the amino acid sequence set forth in SEQ ID NO: 21 (or a variant thereof);
(4) an HCVR that comprises the amino acid sequence set forth in SEQ ID NO: 25 (or a variant thereof); and an LCVR that comprises the amino acid sequence set forth in SEQ ID NO: 29 (or a variant thereof);
(5) an HCVR that comprises the amino acid sequence set forth in SEQ ID NO: 33 (or a variant thereof); and an LCVR that comprises the amino acid sequence set forth in SEQ ID NO: 37 (or a variant thereof);
(6) an HCVR that comprises the amino acid sequence set forth in SEQ ID NO: 41 (or a variant thereof); and an LCVR that comprises the amino acid sequence set forth in SEQ ID NO: 45 (or a variant thereof);
(7) an HCVR that comprises the amino acid sequence set forth in SEQ ID NO: 49 (or a variant thereof); and an LCVR that comprises the amino acid sequence set forth in SEQ ID NO: 53 (or a variant thereof);
(8) an HCVR that comprises the amino acid sequence set forth in SEQ ID NO: 57 (or a variant thereof); and an LCVR that comprises the amino acid sequence set forth in SEQ ID NO: 61 (or a variant thereof);
(9) an HCVR that comprises the amino acid sequence set forth in SEQ ID NO: 65 (or a variant thereof); and an LCVR that comprises the amino acid sequence set forth in SEQ ID NO: 69 (or a variant thereof);
(10) an HCVR that comprises the amino acid sequence set forth in SEQ ID NO: 73 (or a variant thereof); and an LCVR that comprises the amino acid sequence set forth in SEQ ID NO: 77 (or a variant thereof);
(11) an HCVR that comprises the amino acid sequence set forth in SEQ ID NO: 81 (or a variant thereof); and an LCVR that comprises the amino acid sequence set forth in SEQ ID NO: 85 (or a variant thereof); or
(12) an HCVR that comprises the amino acid sequence set forth in SEQ ID NO: 89 (or a variant thereof); and an LCVR that comprises the amino acid sequence set forth in SEQ ID NO: 93 (or a variant thereof)
(13) an HCVR that comprises the amino acid sequence set forth in SEQ ID NO: 97 (or a variant thereof); and an LCVR that comprises the amino acid sequence set forth in SEQ ID NO: 101 (or a variant thereof);
(14) an HCVR that comprises the amino acid sequence set forth in SEQ ID NO: 105 (or a variant thereof); and an LCVR that comprises the amino acid sequence set forth in SEQ ID NO: 109 (or a variant thereof);
(15) an HCVR that comprises the amino acid sequence set forth in SEQ ID NO: 113 (or a variant thereof); and an LCVR that comprises the amino acid sequence set forth in SEQ ID NO: 117 (or a variant thereof);
(16) an HCVR that comprises the amino acid sequence set forth in SEQ ID NO: 121 (or a variant thereof); and an LCVR that comprises the amino acid sequence set forth in SEQ ID NO: 125 (or a variant thereof);
(17) an HCVR that comprises the amino acid sequence set forth in SEQ ID NO: 129 (or a variant thereof); and an LCVR that comprises the amino acid sequence set forth in SEQ ID NO: 133 (or a variant thereof);
(18) an HCVR that comprises the amino acid sequence set forth in SEQ ID NO: 137 (or a variant thereof); and an LCVR that comprises the amino acid sequence set forth in SEQ ID NO: 141 (or a variant thereof);
(19) an HCVR that comprises the amino acid sequence set forth in SEQ ID NO: 429 (or a variant thereof); and an LCVR that comprises the amino acid sequence set forth in SEQ ID NO: 437 (or a variant thereof); or
(20) an HCVR that comprises the amino acid sequence set forth in SEQ ID NO: 451 (or a variant thereof); and an LCVR that comprises the amino acid sequence set forth in SEQ ID NO: 459 (or a variant thereof).

In some embodiments of any of the above-described antigen-binding proteins, the antigen-binding protein comprises:
(a) a heavy chain that comprises the amino acid sequence set forth in SEQ ID NO: 145 (or a variant thereof), and a light chain that comprises the amino acid sequence set forth in SEQ ID NO: 146 (or a variant thereof);
(b) a heavy chain that comprises the amino acid sequence set forth in SEQ ID NO: 147 (or a variant thereof), and a light chain that comprises the amino acid sequence set forth in SEQ ID NO: 148 (or a variant thereof);
(c) a heavy chain that comprises the amino acid sequence set forth in SEQ ID NO: 149 (or a variant thereof), and a light chain that comprises the amino acid sequence set forth in SEQ ID NO: 150 (or a variant thereof);
(d) a heavy chain that comprises the amino acid sequence set forth in SEQ ID NO: 151 (or a variant thereof), and a light chain that comprises the amino acid sequence set forth in SEQ ID NO: 152 (or a variant thereof);
(e) a heavy chain that comprises the amino acid sequence set forth in SEQ ID NO: 153 (or a variant thereof), and a light chain that comprises the amino acid sequence set forth in SEQ ID NO: 154 (or a variant thereof);
(f) a heavy chain that comprises the amino acid sequence set forth in SEQ ID NO: 155 (or a variant thereof), and a light chain that comprises the amino acid sequence set forth in SEQ ID NO: 156 (or a variant thereof);
(g) a heavy chain that comprises the amino acid sequence set forth in SEQ ID NO: 157 (or a variant thereof), and a light chain that comprises the amino acid sequence set forth in SEQ ID NO: 158 (or a variant thereof);
(h) a heavy chain that comprises the amino acid sequence set forth in SEQ ID NO: 159 (or a variant thereof), and a light chain that comprises the amino acid sequence set forth in SEQ ID NO: 160 (or a variant thereof);
(i) a heavy chain that comprises the amino acid sequence set forth in SEQ ID NO: 161 (or a variant thereof), and a light chain that comprises the amino acid sequence set forth in SEQ ID NO: 162 (or a variant thereof);
(j) a heavy chain that comprises the amino acid sequence set forth in SEQ ID NO: 163 (or a variant thereof), and a light chain that comprises the amino acid sequence set forth in SEQ ID NO: 164 (or a variant thereof);
(k) a heavy chain that comprises the amino acid sequence set forth in SEQ ID NO: 165 (or a variant thereof), and a light chain that comprises the amino acid sequence set forth in SEQ ID NO: 166 (or a variant thereof);
(l) a heavy chain that comprises the amino acid sequence set forth in SEQ ID NO: 167 (or a variant thereof), and a light chain that comprises the amino acid sequence set forth in SEQ ID NO: 168 (or a variant thereof);
(m) a heavy chain that comprises the amino acid sequence set forth in SEQ ID NO: 169 (or a variant thereof), and a light chain that comprises the amino acid sequence set forth in SEQ ID NO: 170 (or a variant thereof);
(n) a heavy chain that comprises the amino acid sequence set forth in SEQ ID NO: 171 (or a variant thereof), and a light chain that comprises the amino acid sequence set forth in SEQ ID NO: 172 (or a variant thereof);
(o) a heavy chain that comprises the amino acid sequence set forth in SEQ ID NO: 173 (or a variant thereof), and a light chain that comprises the amino acid sequence set forth in SEQ ID NO: 174 (or a variant thereof);
(p) a heavy chain that comprises the amino acid sequence set forth in SEQ ID NO: 175 (or a variant thereof), and a light chain that comprises the amino acid sequence set forth in SEQ ID NO: 176 (or a variant thereof);
(q) a heavy chain that comprises the amino acid sequence set forth in SEQ ID NO: 177 (or a variant thereof), and a light chain that comprises the amino acid sequence set forth in SEQ ID NO: 178 (or variant thereof);
(r) a heavy chain that comprises the amino acid sequence set forth in SEQ ID NO: 179 (or a variant thereof), and a light chain that comprises the amino acid sequence set forth in SEQ ID NO: 180 (or a variant thereof);
(s) a heavy chain that comprises the amino acid sequence set forth in SEQ ID NO: 443 (or a variant thereof), and a light chain that comprises the amino acid sequence set forth in SEQ ID NO: 447 (or a variant thereof); or
(t) a heavy chain that comprises the amino acid sequence set forth in SEQ ID NO: 465 (or a variant thereof), and a light chain that comprises the amino acid sequence set forth in SEQ ID NO: 469 (or a variant thereof).

In another aspect, provided herein is an antigen-binding protein that binds to the same epitope on CACNG1 as an antibody comprising an HCVR/LCVR amino acid sequence pair as set forth in Table 1-1.

In another aspect, provided herein is an antigen-binding protein that competes for binding to CACNG1 with an antibody comprising an HCVR/LCVR amino acid sequence pair as set forth in Table 1-1.

In some embodiments of any of the above-described antigen-binding proteins, the antigen-binding protein comprises an antibody or antigen-binding fragment thereof.

In some embodiments of any of the above-described antigen-binding proteins, the antigen-binding protein is a humanized antibody or antigen binding fragment thereof, a human antibody or antigen binding fragment thereof, a murine antibody or antigen binding fragment thereof, a chimeric antibody or antigen binding fragment thereof, a monovalent Fab′, a divalent Fab2, an F(ab)′3 fragment, a single-chain fragment variable (scFv), a bis-scFv, a (scFv)2, a diabody, a minibody, a nanobody, a triabody, a tetrabody, a disulfide stabilized Fv protein (dsFv), a single-domain antibody (sdAb), an Ig NAR, a single heavy chain antibody, a bispecific antibody or binding fragment thereof, a bi-specific T-cell engager (BiTE), a trispecific antibody, or a chemically modified derivative thereof.

In some embodiments, the antigen-binding protein comprises a fragment antigen-binding region (Fab).

In some embodiments, the antigen-binding protein comprises a single chain fragment variable (scFv).

In some embodiments, the scFv comprises variable regions arranged in the following orientation from N-terminus to C-terminus: HCVR-LCVR.

In some embodiments, the scFv comprises variable regions arranged in the following orientation from N-terminus to C-terminus: LCVR-HCVR.

In some embodiments, the variable regions in the scFv are connected by a linker.

In some embodiments, the linker is a peptide linker.

In some embodiments, the peptide linker is -(GGGGS)n-(SEQ ID NO: 411), wherein n is any integral selected from 1-10.

In some embodiments of any of the above-described antigen-binding proteins, the antigen-binding protein binds specifically to human CACNG1.

In some embodiments, the antigen-binding protein binds to human hCACNG1 with a K_{D} of about 1×10⁻⁷ M or a stronger affinity.

In some embodiments, the anti-hCACNG1 antibody or antigen-binding fragment thereof binds to hCACNG1 with a K_{D} of about 1×10⁻⁷ to about 1×10⁻¹⁰.

In some embodiments, the anti-hCACNG1 antibody or antigen-binding fragment thereof binds to hCACNG1 with a K_{D} of about 5×10⁻⁹ to about 1×10⁻¹⁰.

In another aspect, provided herein is an isolated polynucleotide encoding an antigen-binding protein described herein.

In another aspect, provided herein is a vector comprising an isolated polynucleotide described herein.

In another aspect, provided herein is a host cell comprising an antigen-binding protein described herein, an isolated polynucleotide described herein, or a vector described herein.

In some embodiments, a host cell described herein can be a Chinese hamster ovary (CHO) cell.

In another aspect, provided herein is a protein-drug conjugate comprising an antigen-binding protein that binds specifically to Calcium Voltage-Gated Channel Auxiliary Subunit Gamma 1 (CACNG1) and is conjugated to a molecular cargo.

In some embodiments of the above-described protein-drug conjugate, the antigen-binding protein comprises any of the above-described antigen-binding proteins.

In some embodiments of any of the above-described protein-drug conjugates, the antigen-binding protein and the molecular cargo are conjugated via a linker.

In some embodiments of any of the above-described protein-drug conjugates, the molecular cargo comprises a polynucleotide molecule, a polypeptide molecule, a carrier, or a small molecule.

In some embodiments, the molecular cargo comprises a polynucleotide molecule.

In some embodiments, the polynucleotide molecule is an interfering nucleic acid molecule, a guide RNA, a ribozyme, an aptamer, a mixmer, a multimer, or an mRNA.

In some embodiments, the interfering nucleic acid molecule is an siRNA, an shRNA, a miRNA, an antisense oligonucleotide, or a gapmer.

In some embodiments, the interfering nucleic acid molecule is an siRNA.

In some embodiments, the siRNA comprises a sense strand of 21 nucleotides in length.

In some embodiments, the siRNA comprises an antisense strand of 23 nucleotides in length.

In some embodiments, the siRNA comprises two phosphorothioate linkages at the first and second internucleoside linkages at the 5′ end of the sense strand.

In some embodiments, the siRNA comprises two phosphorothioate linkages at the first and second internucleoside linkages at the 3′ and/or 5′ ends of the antisense strand.

In some embodiments, the interfering nucleic acid is an antisense oligonucleotide.

In some embodiments, the polynucleotide molecule is a guide RNA.

In some embodiments, the polynucleotide molecule targets a gene or gene product associated with a skeletal muscle disease or disorder.

In some embodiments, the gene or gene product associated with a skeletal muscle disease or disorder is Double Homeobox 4 (DUX4), myotonic dystrophy protein kinase (DMPK), dystrophin (DMD), F-Box Only Protein 32 (FBX032), Tripartite Motif Containing 63 (TRIM63), Inhibin Subunit Beta A (INHBA), Myostatin (MSTN), Myocyte Enhancer Factor 2D (MEF2D), KLF Transcription Factor 15 (KLF15), Mediator Complex Subunit 1 (MED1), Mediator Complex Subunit 13 (MED13), Protein Phosphatase 1 Regulatory Subunit 3A (PPP1R3A), Myosin Light Chain Kinase (MLCK1), Activin A Receptor Type 1B (ACVR1 B), Type II SH2-domain-containing inositol 5-phosphatase (SHIP2), or a gene disclosed in Table 1-3.

In some embodiments, the polynucleotide molecule comprises one or more modified nucleotides.

In some embodiments, the molecular cargo comprises a polypeptide molecule.

In some embodiments, the polypeptide molecule is an enzyme or an antigen-binding protein that binds to a target other than CACNG1.

In some embodiments, the polypeptide molecule is associated with a skeletal muscle disease or disorder.

In some embodiments, the molecular cargo comprises a small molecule.

In some embodiments, the small molecule is an androgen, a glucocorticoid, a β2-adrenergic receptor agonist, rapamycin or an analog thereof, a MAPK inhibitor, or a histone deacetylase inhibitor.

In some embodiments, the small molecule is an androgen.

In some embodiments, the androgen is dihydrotestosterone (DHT).

In some embodiments, the small molecule is a glucocorticoid.

In some embodiments, the glucocorticoid is budesonide.

In some embodiments, the antigen-binding protein and the small molecule are conjugated via a valine-citrulline para-aminobenzylcarbamate (VC-PAB) and/or a glutamic acid-valine-citrulline para-aminobenzylcarbamate (EVC-PAB) linker.

In some embodiments, any of the above-described protein-drug conjugates can be used in treating, preventing, or reducing the likelihood of a skeletal muscle disease or disorder.

In some embodiments, the skeletal muscle disease or disorder is a muscular dystrophy, a muscular atrophy, an inflammatory myopathy, a disease of the peripheral nerve, a disease of the neuromuscular junction, a metabolic disease of the muscle, central core disease, hyperthyroid myopathy, myotonia congenita, myotubular myopathy, Nemaline myopathy, paramyotonia congenita, periodic paralysis-hypokalemic-hyperkalemic, centronuclear myopathy, Laing distal myopathy, myofibrillar myopathy, or a disease or disorder disclosed in Tables 1-3.

In some embodiments, the muscular dystrophy is Duchenne muscular dystrophy (DMD), Becker muscular dystrophy (BMD), a congenital muscular dystrophy, a distal muscular dystrophy, Emery-Dreifuss muscular dystrophy, a facioscapulohumeral muscular dystrophy, a Limb-Girdle muscular dystrophy, a myotonic muscular dystrophy, or an oculopharyngeal muscular dystrophy.

In some embodiments, the muscular atrophy is a spinal muscular atrophy, or a muscular atrophy induced by cancer cachexia, disuse, heart failure, chronic obstructive pulmonary disease, or a chronic infection.

In some embodiments, the spinal muscular atrophy is Amyotrophic Lateral Sclerosis (ALS), infantile progressive spinal muscular atrophy, intermediate spinal muscular atrophy, juvenile spinal muscular atrophy, or adult spinal muscular atrophy.

In some embodiments, the inflammatory myopathy is dermatomyositis, polymyositis, or inclusion body myositis.

In some embodiments, the disease of the peripheral nerve is Charcot-Marie tooth disease, Dejerine-Sottas disease, or Friedreich's ataxia.

In some embodiments, the disease of the neuromuscular junction is Myasthenia gravis, Lambert-Eaton syndrome, or botulism.

In some embodiments, the metabolic disease of the muscle is acid maltase deficiency, carnitine deficiency, carnitine palmityl transferase deficiency, debrancher enzyme deficiency, lactate dehydrogenase deficiency, mitochondrial myopathy, myoadenylate deaminase deficiency, phosphorylase deficiency, phosphofructokinase deficiency, or phosphoglycerate kinase deficiency.

In some embodiments, the polypeptide molecule is Double Homeobox 4 (DUX4), myotonic dystrophy protein kinase (DMPK), dystrophin (DMD), F-Box Only Protein 32 (FBX032), Tripartite Motif Containing 63 (TRIM63), Inhibin Subunit Beta A (INHBA), Myostatin (MSTN), Myocyte Enhancer Factor 2D (MEF2D), KLF Transcription Factor 15 (KLF15), Mediator Complex Subunit 1 (MED1), Mediator Complex Subunit 13 (MED13), Protein Phosphatase 1 Regulatory Subunit 3A (PPP1R3A), Myosin Light Chain Kinase (MLCK1), Activin A Receptor Type 1B (ACVR1 B), Type-II SH2-domain-containing inositol 5-phosphatase (SHIP2), or a protein disclosed in Tables 1-3.

In some embodiments, the molecular cargo comprises a carrier.

In some embodiments, the carrier is a lipid-based carrier.

In some embodiments, the lipid-based carrier is a lipid nanoparticle (LNP), a liposome, a lipidoid, or a lipoplex.

In some embodiments, the lipid-based carrier is a lipid nanoparticle (LNP).

In some embodiments, the LNP further comprises a polynucleotide molecule and/or a polypeptide molecule.

In some embodiments, the LNP comprises one or more components of a gene editing system.

In some embodiments, an LNP described herein comprises:
(a) a Cas nuclease, or a nucleic acid encoding the Cas nuclease, and/or
(b) a guide RNA, or one or more DNAs encoding the guide RNA.

In some embodiments, the Cas nuclease is a Cas9 protein.

In some embodiments, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

In some embodiments, the nucleic acid encoding the Cas nuclease is codon-optimized for expression in a mammalian cell.

In some embodiments, the nucleic acid encoding the Cas nuclease is codon-optimized for expression in a human cell.

In some embodiments, the nucleic acid encoding the Cas nuclease is an mRNA.

In some embodiments, the guide RNA is a single guide RNA (sgRNA).

In some embodiments, an LNP described herein comprises a zinc finger nuclease (ZFN) or a transcription activator-like effector nuclease (TALEN).

In some embodiments, the LNP comprises a cationic lipid, a neutral lipid, a helper lipid, a stealth lipid, or any combination thereof.

In some embodiments, the neutral lipid is distearoylphosphatidylcholine (DSPC).

In some embodiments, the helper lipid is cholesterol.

In some embodiments, the stealth lipid is PEG2k-DMG.

In another aspect, provided herein is a pharmaceutical composition comprising an antigen-binding protein described herein, an isolated polynucleotide described herein, a vector described herein, or a protein-drug conjugate described herein, and a pharmaceutically acceptable carrier.

In another aspect, provided herein is a composition or kit comprising an antigen-binding protein described herein, an isolated polynucleotide described herein, a vector described herein, a protein-drug conjugate described herein, or a pharmaceutical described herein, and a further therapeutic agent.

In another aspect, provided herein is a complex comprising an antigen-binding protein described herein or a protein-drug conjugate described herein bound to Calcium Voltage-Gated Channel Auxiliary Subunit Gamma 1 (CACNG1).

In another aspect, provided herein is a method for making an antigen-binding protein described herein, comprising culturing a host cell comprising a polynucleotide that encodes the antigen-binding protein in a culture medium under conditions favorable for expression of the antigen-binding protein.

In some embodiments of the above-described method for making an antigen-binding protein, the method comprises the steps:
(a) introducing the polynucleotide into a host cell;
(b) culturing the host cell under conditions favorable for expression of the antigen-binding protein;
(c) optionally, isolating the antigen-binding protein from the culture medium and/or host cell; and
(d) optionally, conjugating the antigen-binding protein to a molecular cargo.

In another aspect, provided herein is an antigen-binding protein which is produced by or obtainable by any of the above-described methods for making an antigen-binding protein.

In another aspect, provided herein is a method for making a protein-drug conjugate described herein comprising:
(a) contacting the antigen-binding protein, with the molecular cargo under the conditions favorable for conjugation of the antigen-binding protein to the molecular cargo; and
(b) optionally, isolating the protein-drug conjugate produced in step (a).

In another aspect, provided herein is a method for making a protein-drug conjugate of described herein, wherein the molecular cargo comprises a polypeptide molecule, the method comprising:
(a) culturing a host cell comprising a polynucleotide encoding the protein-drug conjugate under conditions that allow expression of the protein-drug conjugate; and
(b) optionally, isolating the protein-drug conjugate produced in step (a).

In another aspect, provided herein is a protein-drug conjugate produced by or obtainable by any of the above-described methods for making a protein-drug conjugate.

In another aspect, provided herein is a vessel or injection device comprising an antigen-binding protein described herein, an isolated polynucleotide described herein, a vector described herein, or a protein-drug conjugate described herein.

In another aspect, provided herein is a method for imaging skeletal muscle in a subject in need thereof, comprising introducing an antigen-binding protein described herein into the body of the subject, wherein the antigen-binding protein is conjugated to a detectable biosensor or a radioactive isotope.

In some embodiments, the radioactive isotope comprises a radionuclide.

In some embodiments, the antigen-binding protein conjugated to a detectable biosensor or a radioactive isotope is introduced to the subject via intramuscular, intravenous or subcutaneous administration.

In another aspect, provided herein is a method for causing internalization of a small molecule by a myofiber, comprising contacting the myofiber with an antigen-binding protein described herein, and wherein the antigen-binding protein is conjugated to a small molecule.

In some embodiments, the small molecule is an androgen, a glucocorticoid, a β2-adrenergic receptor agonist, rapamycin or an analog thereof, a MAPK inhibitor, or a histone deacetylase inhibitor.

In some embodiments, the small molecule is an androgen.

In some embodiments, the androgen is dihydrotestosterone (DHT).

In some embodiments, the small molecule is a glucocorticoid.

In some embodiments, the glucocorticoid is budesonide.

In some embodiments, the small molecule comprises a detectable biosensor or a radioactive isotope.

In some embodiments, the detectable radioactive isotope moiety comprises a radionuclide.

In some embodiments of any of the above-described methods for causing internalization of a small molecule by a myofiber, the contacting comprises administering intramuscularly, intravenously or subcutaneously to a subject in need thereof the antigen-binding protein conjugated to the small molecule.

In some embodiments of any of the above-described methods for causing internalization of a small molecule by a myofiber, wherein the contacting comprises culturing the myofiber in vitro with the antigen-binding protein conjugated to the small molecule.

In some embodiments of any of the above-described methods for causing internalization of a small molecule by a myofiber, the antigen-binding protein is conjugated to the small molecule via a valine-citrulline para-aminobenzylcarbamate (VC-PAB) and/or a glutamic acid-valine-citrulline para-aminobenzylcarbamate (EVC-PAB) linker.

In another aspect, provided herein is a method for administering an antigen-binding protein described herein, an isolated polynucleotide described herein, a vector described herein, or a protein-drug conjugate described herein to a subject in need thereof, the method comprising introducing the antigen-binding protein, the polynucleotide, the vector, or the protein-drug conjugate into the body of the subject.

In some embodiments, the antigen-binding protein, the polynucleotide, the vector, or the protein-drug conjugate is introduced into the body of the subject via intramuscular, subcutaneous, or intravenous administration.

In another aspect, provided herein is a method for delivering a molecular cargo to a skeletal muscle tissue and/or cell in the body of a subject in need thereof comprising administering to the subject a protein-drug conjugate described herein or a pharmaceutical described herein.

In another aspect, provided herein is a method for treating, preventing, or reducing the likelihood of a skeletal muscle disease or disorder in a subject in need thereof comprising administering to the subject a therapeutically-effective amount of an antigen-binding protein described herein, an isolated polynucleotide described herein, a vector described herein, a protein-drug conjugate described herein, or a pharmaceutical composition described herein.

In some embodiments of the above-described method for treating, preventing, or reducing the likelihood of a skeletal muscle disease or disorder in a subject in need thereof, the antigen-binding protein, the polynucleotide, the vector, or the protein-drug conjugate is administered via intramuscular, subcutaneous, or intravenous administration.

In some embodiments, the skeletal muscle disease or disorder is a muscular dystrophy, a muscular atrophy, an inflammatory myopathy, a disease of the peripheral nerve, a disease of the neuromuscular junction, a metabolic disease of the muscle, central core disease, hyperthyroid myopathy, myotonia congenita, myotubular myopathy, Nemaline myopathy, paramyotonia congenita, periodic paralysis-hypokalemic-hyperkalemic, centronuclear myopathy, Laing distal myopathy, myofibrillar myopathy, or a disease or disorder disclosed in Tables 1-3.

In some embodiments, the muscular dystrophy is Duchenne muscular dystrophy (DMD), Becker muscular dystrophy (BMD), a congenital muscular dystrophy, a distal muscular dystrophy, Emery-Dreifuss muscular dystrophy, a facioscapulohumeral muscular dystrophy, a Limb-Girdle muscular dystrophy, a myotonic muscular dystrophy, or an oculopharyngeal muscular dystrophy.

In some embodiments, the muscular atrophy is a spinal muscular atrophy, or a muscular atrophy induced by cancer cachexia, disuse, heart failure, chronic obstructive pulmonary disease, or a chronic infection.

In some embodiments, the spinal muscular atrophy is Amyotrophic Lateral Sclerosis (ALS), infantile progressive spinal muscular atrophy, intermediate spinal muscular atrophy, juvenile spinal muscular atrophy, or adult spinal muscular atrophy.

In some embodiments, the inflammatory myopathy is dermatomyositis, polymyositis, or inclusion body myositis.

In some embodiments, the disease of the peripheral nerve is Charcot-Marie tooth disease, Dejerine-Sottas disease, or Friedreich's ataxia.

In some embodiments, the disease of the neuromuscular junction is Myasthenia gravis, Lambert-Eaton syndrome, or botulism.

In some embodiments, the metabolic disease of the muscle is acid maltase deficiency, carnitine deficiency, carnitine palmityl transferase deficiency, debrancher enzyme deficiency, lactate dehydrogenase deficiency, mitochondrial myopathy, myoadenylate deaminase deficiency, phosphorylase deficiency, phosphofructokinase deficiency, or phosphoglycerate kinase deficiency.

In some embodiments of any of the above-described methods for treating, preventing, or reducing the likelihood of a skeletal muscle disease or disorder in a subject in need thereof, the method can further comprise administering an additional treatment to the subject.

In some embodiments, the additional treatment comprises physical exercise.

In some embodiments, the additional treatment comprises administering a testosterone and/or a glucocorticoid.

### BRIEF DESCRIPTION OF THE FIGURES

FIG. 1 depicts human myotube acetylcholine-induced calcium flux following addition of Calcium Voltage-Gated Channel Auxiliary Subunit Gamma 1 (CACNG1) antibodies, isotype control antibodies, or nicardipine (positive control for calcium blocking).

FIG. 2 shows binding of CACNG1 antibodies to single myofibers ex vivo.

FIG. 3 illustrates internalization of a fluorophore-conjugated CACNG1 antibody in single myofibers ex vivo.

FIG. 4 shows in vivo CACNG1 antibody biodistribution assessed by cryo-fluorescence tomography.

FIGS. 5A-5G show in vivo CACNG1 antibody biodistribution to skeletal muscles assessed by immunofluorescence imaging of tissue sections obtained from gastrocnemius/plantaris/soleus complex (FIG. 5A), tibialis anterior (FIG. 5B), diaphragm (FIG. 5C), tongue (FIG. 5D), triceps (FIG. 5E), trapezius (FIG. 5F), and pelvic floor muscles (FIG. 5G).

FIGS. 6A-6D show in vivo antibody biodistribution to non-muscle tissues assessed by immunofluorescence imaging of tissue sections obtained from liver (FIG. 6A), kidney (FIG. 6B), spleen (FIG. 6C), and brown adipose (FIG. 6D).

FIG. 7 demonstrates CACNG1 antibody distribution to muscle is altered by exercise and dose. Schematic depicting an exemplary experimental timeline (top panel). Photomicrograph showing CACNG1 antibody distribution to the soleus muscle under sedentary and exercise conditions at either a 10 mg/kg or a 50 mg/kg (high) dose (bottom panel).

FIGS. 8A-8B illustrate CACNG1 is highly and specifically expressed in human skeletal muscle tissue.

FIGS. 9A-9C demonstrate CACNG1 does not regulate skeletal muscle size or function. CACNG1 knockout mice are indistinguishable from wildtype mice with regard to muscle size (FIG. 9A) and muscle function (FIG. 9B). Bar graphs of unaltered muscle twitch (1 Hz) and tetanic (125 Hz) contractile force properties of muscle from wildtype versus CACNG1 knockout mice are shown in FIG. 9C. TA, Tibialis anterior; GA, Gastrocnemius; EDL, extensor digitorum longus.

FIGS. 10A-10D show in vitro and ex vivo evaluation of CACNG1 antibody properties. Mouse and human myotubes were used as an in vitro model of muscle to evaluate CACNG1 antibody cell binding (FIGS. 10A-10B), internalization (FIG. 10C), and localization (FIG. 10D). Incubation of live myotubes with anti-CACNG1 antibodies followed by fluorophore-conjugated secondary detection was performed to assess antibody binding (FIGS. 10A-10B). Incubation of myotubes with anti-CACNG1 antibodies, e.g., REGN7854 and other anti-CACNG1 antibodies described herein, followed by duocarmycin-conjugated secondary (2° Ab-cytotoxic drug) was performed to assess antibody internalization via cell kill (FIG. 10C). Immunostaining of CACNG1 in CACNG1^{Hu/Hu} mouse single myofibers (FIG. 10D, left panel) and muscle tissue cross sections (FIG. 10D, right panel) show expression of CACNG1 at the myofiber cell surface.

FIGS. 11A-11K demonstrate anti-CACNG1-DM1 Protein Kinase (DMPK) siRNA conjugate-specific knockdown of DMPK in skeletal muscle as compared to other tissues. A schematic representation of an exemplary anti-CAGNG1-DMPK siRNA conjugate is shown in FIG. 11A. Graphs showing DMPK mRNA expression levels (relative to PBS) measured in gastrocnemius (FIG. 11B), soleus (FIG. 11C), tibialis anterior (FIG. 11D), quadriceps (FIG. 11E), diaphragm (FIG. 11F), heart (FIG. 11G), liver (FIG. 11H), kidney (FIG. 111), spleen (FIG. 11J), and lungs (FIG. 11K) at 0.3 mg/kg, 1 mg/kg, and 3 mg/kg doses of total siRNA are shown in FIGS. 11B-11K.

FIGS. 12A-12B show a schematic representation of an exemplary siRNA against DMPK (siRNA1).

FIGS. 13A-13E illustrate α-CACNG1-Dmpk siRNA conjugates knock down Dmpk in skeletal muscle 1 week after dosing.

FIGS. 14A-14C illustrate α-CACNG1-Dmpk siRNA conjugates do not knock down Dmpk in other tissues (e.g., liver, heart, kidney) 1 week after dosing.

FIGS. 15A-15E illustrate α-CACNG1-Dmpk siRNA conjugates knock down Dmpk in skeletal muscle 3 weeks after dosing (*p<0.05, **p<0.01, ***p<0.001, ****p<0.0001). TA, tibialis anterior; Quad, quadriceps; Gastroc, gastrocnemius.

FIGS. 16A-16E illustrate α-CACNG1-Dmpk siRNA conjugates knock down Dmpk in skeletal muscle 6 weeks after dosing (*p<0.05, **p<0.01, ***p<0.001, ****p<0.0001). TA, tibialis anterior; Quad, quadriceps; Gastroc, gastrocnemius.

FIGS. 17A-17C illustrate α-CACNG1-Dmpk siRNA conjugates do not knock down Dmpk in other tissues (e.g., heart, liver, kidney) 3 weeks after dosing.

FIGS. 18A-18C illustrate α-CACNG1-Dmpk siRNA conjugates do not knock down Dmpk in other tissues (e.g., heart, liver, kidney) 6 weeks after dosing.

FIG. 19 shows an example of an antibody-steroid conjugation scheme described herein.

FIG. 20 shows a preparative size-exclusion chromatography (SEC) chromatogram of a CACNG1-linker conjugation mixture.

FIG. 21 shows a preparative SEC chromatogram of a CACNG1-steroid conjugation mixture.

FIG. 22 shows an analytical SEC chromatogram of a purified CACNG1-steroid antibody-drug conjugates (ADCs).

FIG. 23 shows a liquid chromatography-electrospray ionization-mass spectrometry (LC-ESI-MS) spectrum of a CACNG1-steroid ΔΔCt C sample. The calculated average drug-to-antibody ration (DAR) value was 3.94.

FIG. 24 shows the level of androgen receptor (AR) activation in terms of relative light units (RLU; y-axis) after a 24 hour incubation of an LNCaP cell line modified to express luciferase upon androgen receptor activation (AR.Luc) with: dihydrotestosterone (DHT) alone (M608; unconjugated DHT); an anti-human CACNG1 (hCACNG1) antibody (REGN14570, REGN14571, REGN14572, REGN14573, REGN14574 or REGN14647) conjugated via a VC-PAB linker to DHT (M3004); or an anti-FelD isotype control antibody (REGN3892) conjugated via a VC-PAB linker to DHT (M3004); at varying concentrations (Log[Conc. (M)]; x-axis).

FIGS. 25A-251 shows the level of androgen receptor (AR) activation in terms of relative light units (RLU; y-axis) after a 24 hour, 48 hour, or 72 hour incubation of a hCACNG1 expressing LNCaP cell line modified to also express luciferase upon androgen receptor activation (hCACNG1.AR.Luc) with: dihydrotestosterone (DHT) alone (M608; unconjugated DHT); an anti-hCACNG1 antibody (REGN14570, REGN14571, REGN14572, REGN14573, REGN14574 or REGN14647) conjugated via a VC-PAB linker to DHT (M3004); or an anti-FelD isotype control antibody (REGN3892) conjugated via a VC-PAB linker to DHT (M3004); at varying concentrations (Log[Conc. (M)]; x-axis).

FIGS. 26A-26C illustrate CACNG1 antibodies (Abs) conjugated to budesonide increase expression of glucocorticoid responsive genes kidney-enriched krueppel-like factor 15 (Klf15) (FIG. 26A), pyruvate dehydrogenase kinase 4 (Pdk4) (FIG. 26B), and FKBP prolyl isomerase 5 (Fkbp5) (FIG. 26C) in C2C12 myotubes.

FIGS. 27A-27D demonstrate anti-CACNG1 biosensor binding and cleavage in primary human skeletal myotubes (HuSKM) and in primary mouse myotubes (C2C12).

FIGS. 28A-28D illustrate quantification of anti-CACNG1 biosensor cleavage in primary human skeletal myotubes (HuSKM) and in primary mouse myotubes (C2C12).

### DETAILED DESCRIPTION OF THE INVENTION

The present disclosure provides antigen-binding proteins that specifically bind to Calcium Voltage-Gated Channel Auxiliary Subunit Gamma 1 (CACNG1), or antigenic fragments thereof. The present disclosure further provides protein-drug conjugates comprising an antigen-binding protein that specifically binds to CACNG1, or an antigenic fragment thereof, and that is conjugated to a molecular cargo. Such conjugates are useful, for example, for delivery of the molecular cargo to skeletal muscle tissue and/or cells (e.g., myofibers) in the body. The delivery of molecular cargos using protein-drug conjugates comprising CACNG1 antigen-binding proteins disclosed herein may be particularly advantageous in such instances where it is specifically desirable to target skeletal muscle tissues and the cells residing therein while avoiding the targeting of non-skeletal muscle tissues (off-targets) including other muscle tissues such as but not limited to smooth muscle tissues. The conjugates described herein have an ability to efficiently deliver molecular cargoes to skeletal muscle tissue and the cells residing therein and, thus, can be used for treatment, prevention, or reduction of the likelihood of diseases and disorders such as skeletal muscle diseases and disorders.

Skeletal muscle is the largest organ in the body, comprising ˜40% of total body mass. Skeletal muscle is one of the three significant muscle tissues in the human body. Each skeletal muscle consists of thousands of muscle fibers wrapped together by connective tissue sheaths. The individual bundles of muscle fibers in a skeletal muscle are known as fasciculi. The outermost connective tissue sheath surrounding the entire muscle is known as epimysium. The connective tissue sheath covering each fasciculus is known as perimysium, and the innermost sheath surrounding individual muscle fiber is known as endomysium. Each muscle fiber is comprised of a number of myofibrils containing multiple myofilaments.

When bundled together, all the myofibrils are arranged in a unique striated pattern forming sarcomeres which are the fundamental contractile unit of a skeletal muscle. The two most significant myofilaments are actin and myosin filaments arranged distinctively to form various bands on the skeletal muscle.

The primary functions of the skeletal muscle take place via its intrinsic excitation-contraction coupling process. As the muscle is attached to the bone tendons, the contraction of the muscle leads to movement of that bone that allows for the performance of specific movements. The skeletal muscle also provides structural support and helps in maintaining the posture of the body. The skeletal muscle also acts as a storage source for amino acids that can be used by different organs of the body for synthesizing organ-specific proteins. The skeletal muscle also acts as a site of glucose disposal in the form of muscle glycogen. The skeletal muscle also plays a central role in maintaining thermostasis and acts as an energy source during starvation. Thus, skeletal muscle plays key roles in locomotion, thermoregulation, and in controlling whole body metabolism.

In many muscle diseases as well as during normal aging, the size and function of skeletal muscle tissue is reduced, resulting in impaired functional mobility, and, in the case of severe muscle diseases, long-term disability and early mortality.

Treatments for muscle wasting (also referred to as muscle atrophy or muscular atrophy herein) and genetic muscle diseases described herein typically consist of broad-acting therapies, such as testosterone or dihydrotestosterone (DHT) therapy for muscle wasting, glucocorticoids (e.g., budesonide) for muscular dystrophies, etc. Untargeted delivery of these therapies reduces efficiency of specific muscle uptake, while also causing significant detrimental off-target effects on other organs.

In some aspects, the present disclosure addresses a need in the art for anti-human antibodies, capable of binding a muscle-specific marker (e.g., CACNG1) and effecting the internalization by muscle cells of a therapeutic payload.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

The term “about,” when used in reference to a particular recited numerical value, means that the value may vary from the recited value by no more than 1%. For example, the expression “about 100” includes 99 and 101 and all values in between (e.g., 99.1, 99.2, 99.3, 99.4, etc.).

A polynucleotide includes DNA and RNA. The present disclosure includes any polynucleotide described herein which is operably linked to a promoter or other expression control sequence.

The term “CACNG1” refers to Calcium Voltage-Gated Channel Auxiliary Subunit Gamma 1. Voltage-dependent calcium channels are generally composed of five subunits. The protein encoded by the CACNG1 gene represents the γ subunit of these subunits. “CACNG1” includes a protein encoded by the CACNG1 gene, and is one of two known gamma subunit proteins. CACNG1 is part of the skeletal muscle 1,4-dihydropyridine-sensitive calcium channel and is an integral membrane protein that plays a role in excitation-contraction coupling. CACNG1 is part of a functionally diverse eight-member protein subfamily of the PMP-22/EMP/MP20 family and is located in a cluster with two family members that function as transmembrane AMPA receptor regulatory proteins (TARPs). CACNG1 is highly and specifically expressed in skeletal muscle. The gene encoding human CACNG1 (CACNG1) is located on the long arm of chromosome 17. CACNG1 comprises 4 exons and is approximately 12,244 bases long. An example sequence for a human CACNG1 mRNA is assigned NCBI Accession Number NM_000727.4:

(SEQ ID NO: 473)
ACTCCCAGCTCGACAACCACTGCCACCCCCCAAGCTCGGCTTGTCAC
CTGCCCTAGGAGACGCAGCCGCCGGACCCTGCCCAGGGCACCCACGC
CTCGGCGACCACCATGTCCCAGACCAAAATGCTGAAGGTCCGCGTGA
CCCTCTTCTGCATCCTGGCAGGCATCGTGCTGGCCATGACAGCCGTG
GTAACCGACCACTGGGCTGTGCTGAGCCCCCACATGGAGCACCACAA
CACTACCTGCGAGGCGGCCCACTTCGGCCTCTGGCGGATTTGTACCA
AGCGCATCCCCATGGACGACAGCAAGACCTGCGGGCCCATCACCCTG
CCCGGGGAGAAGAACTGTTCCTACTTCAGGCATTTTAACCCCGGCGA
GAGCTCGGAGATCTTCGAATTCACCACTCAGAAGGAGTACAGCATCT
CGGCAGCCGCCATCGCCATCTTCAGCCTTGGCTTCATCATCCTGGGC
AGCCTCTGTGTCCTCCTGTCCCTCGGGAAGAAGAGGGACTATCTGCT
GCGACCCGCGTCCATGTTCTATGCCTTTGCAGGTCTCTGCATCCTCG
TCTCGGTGGAGGTCATGCGGCAGTCGGTGAAGCGCATGATTGACAGT
GAGGACACCGTCTGGATCGAGTACTATTACTCCTGGTCCTTTGCCTG
CGCCTGTGCCGCCTTCATCCTCCTCTTTCTCGGCGGTCTCGCCCTCC
TGCTGTTCTCCCTGCCTCGAATGCCCCGGAACCCATGGGAGTCCTGC
ATGGATGCTGAGCCCGAGCACTAACCCTCCTGCGGCCCTAGCGACCC
TCAGGCTTCTTCCCCAGGAAGCGGGGTCTTGGCCTGGAACCTTCCAG
AGAGGAGGCGGGAGCAATTTTAGCCCCACCCTGCTCCCATCTGCCCC
CCTGCAACAGTCGCAGGCTGCTTCCTCTCTCTGAGTTCCTCTGGGCT
GCCGCAGGCTCCCCTGGGAATAGAGCAAGACGTGAGTCCTAACCTGG
CCACAGTTGGGGGAGGCAGAGCCAGCAGGTGGACAGGTGTTTGCAGG
GGCCCAACTTCCCCTGGAGCTCAGAGGTGTCCCCACTGTACCAGCCT
CTGATAAGCTGCCTCCAGTTGTCCTTTATGAACATTGCAGGGACAAC
CTGTGTTTGCCAGCTGGGTGTTCCGTGTAAATAGCCAGCCTGTCTCT
TTCTCGGTGATAAAACACACCCTCTCTGGTGAGCCCAGCGTCCCCTC
CTTGGCTTCCAGGAGCCCTGGGAAGCATTTTTAACTGGGTAGAATCT
GACTGTGGCTTGAAATAAAAAGCTCTCAGAAAA

An example human CACNG1 protein is assigned NCBI Accession Number NP_000718:

(SEQ ID NO: 474)
MSQTKMLKVRVTLFCILAGIVLAMTAVVTDHWAVLSPHMEHHNTTCE
AAHFGLWRICTKRIPMDDSKTCGPITLPGEKNCSYFRHFNPGESSEI
FEFTTQKEYSISAAAIAIFSLGFIILGSLCVLLSLGKKRDYLLRPAS
MFYAFAGLCILVSVEVMRQSVKRMIDSEDTVWIEYYYSWSFACACAA
FILLFLGGLALLLFSLPRMPRNPWESCMDAEPEH

The antigen-binding proteins (e.g., antibodies and antigen-binding fragments) as described herein may bind soluble CACNG1 and/or cell surface expressed CACNG1. Soluble CACNG1 includes natural CACNG1 proteins as well as recombinant CACNG1 protein variants that lack a transmembrane domain or are otherwise unassociated with a cell membrane.

The expression “cell surface-expressed CACNG1” refers to one or more CACNG1 protein(s) that is/are expressed on the surface of a cell in vitro or in vivo, such that at least a portion of a CACNG1 protein is exposed to the extracellular side of the cell membrane and is accessible to an antigen-binding portion of an antibody. A “cell surface-expressed CACNG1” can comprise or consist of a CACNG1 protein expressed on the surface of a cell which normally expresses CACNG1 protein. Alternatively, “cell surface-expressed CACNG1” can comprise or consist of a CACNG1 protein expressed on the surface of a cell that normally does not express human CACNG1 on its surface but has been artificially engineered to express CACNG1 on its surface.

CACNG1 Binding Proteins and Protein-Drug Conjugates

In one aspect, the present disclosure provides antigen-binding proteins that bind specifically to CACNG1.

An antigen-binding protein that specifically binds to CACNG1 may bind at about 25° C., to CACNG1 or a fusion protein thereof, for example, a tag such as PΔΔCt RE-Flag-His fused to e.g., human CACNG1 in a surface plasmon resonance assay, with a K_{D} of about 1×10⁷ M or a stronger affinity. Such an antigen-binding protein may be referred to as “anti-CACNG1”.

In some embodiments, the antigen-binding protein that specifically binds to CACNG1 may comprise an antibody, or an antigen-binding fragment of an antibody, such as a fragment antigen-binding region (Fab) or single chain fragment variable (scFv).

The present disclosure provides CACNG1 binding protein-drug conjugates. A CACNG1 binding protein-drug conjugate comprises an optional signal peptide, connected to an antigen-binding protein (e.g., an antibody or an antigen-binding fragment of an antibody such as a fragment antigen-binding region (Fab) or single chain fragment variable (scFv)) that binds specifically to CACNG1, such as human CACNG1, and that is conjugated (optionally by a linker) to molecular cargo. The CACNG1-binding protein-drug conjugates described herein can deliver the conjugated molecular cargo to a desired tissue (e.g., skeletal muscle tissue) and/or desired cell type (e.g., myofibers) in the body.

The term “conjugate” means a body in which two substances are linked covalently, or non-covalently. The term “covalently linked” refers to a characteristic of at least two molecules being linked together by way of one or more covalent bond(s). In various embodiments, two molecules can be covalently linked together by a single bond, e.g., a disulfide bridge or a disulfide bond, that operates as a linker between the molecules. In some embodiments, two or more molecules may be covalently linked together by way of a molecule that operates as a linker that joins the at least two molecules together via multiple covalent bonds. In certain embodiments, a linker can be a cleavable linker or a non-cleavable linker. In the conjugate, the two substances may be linked directly or may be linked via a linker. In the present disclosure, one of the two substances is an antigen-binding protein, e.g., an antibody or antigen-binding fragment thereof, and the other is a drug (e.g., a polynucleotide, a polypeptide, a small molecule, a liposome or an LNP disclosed herein). In the present disclosure, the linker may be a cleavable linker or may be a non-cleavable linker. In some embodiments, two polypeptide molecules that are covalently linked, either directly or indirectly (e.g., by a linker), may be expressed from one single polynucleotide molecule.

As used herein, the term “antibody-drug conjugate” or “ADC” means a conjugate of an antibody or antigen-binding fragment thereof with a drug (e.g., a polynucleotide, a polypeptide, a small molecule, a liposome or an LNP disclosed herein). The affinity to an antigen is imparted to a drug by linking an antibody or antigen-binding fragment thereof with the drug (e.g., a polynucleotide, or a liposome or LNP disclosed herein), thereby increasing the efficiency of delivering the drug to a target site in vivo. “Antibody-drug conjugates” or “ADCs” as used herein also encompass fusion proteins wherein the antibody or antigen-binding fragment thereof is fused with another polypeptide molecule.

Antigen-binding molecules described herein includes an antibody and an antigen-binding fragment of an antibody. Accordingly, the present disclosure includes antibodies and antigen-binding fragments thereof, such as Fabs and scFvs, that bind specifically to the CACNG1, such as human CACNG1.

An antibody described herein can be any antigen-binding molecule or molecular complex comprising at least one complementarity determining region (CDR) that specifically binds to or interacts with a particular antigen (e.g., CACNG1). In some embodiments, the term “antibody” refers to immunoglobulin molecules comprising four polypeptide chains, two heavy chains (HCs) and two light chains (LCs), inter-connected by disulfide bonds (e.g., IgG). In an embodiment, each antibody heavy chain (HC) comprises a heavy chain variable region (“HCVR” or “VH”) and a heavy chain constant region which can comprise three domains CH1, CH2 and CH3; and each antibody light chain (LC) comprises a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL comprises three CDRs and four FRs. The three CDRs and the four FRs can be arranged from amino-terminus to carboxy-terminus in the following order: FR1, CDR1, FR2, CDR2, FR3, CDR3, FR4 (heavy chain CDRs may be abbreviated as HCDR1, HCDR2, and HCDR3; light chain CDRs may be abbreviated as LCDR1, LCDR2, and LCDR3. The term “antibody” also includes antigen-binding fragments of full antibody molecules.

An antibody may encompass any type of antibody, such as, e.g., monoclonal or polyclonal. Moreover, the antibody may be or any origin, such as, e.g., mammalian or non-mammalian. In one embodiment, the antibody may be mammalian or avian. In a further embodiment, the antibody may be of human origin and may further be a human monoclonal antibody.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a K_{D} in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that may be useful include e.g., those, that do not selectively bind either the first or second antigen selectively bound by the antigen-binding protein. Suitable light chains include those that can be identified by screening for the most com-monly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a ca-nonical domain (VH or VL) having a dual beta sheet structure wherein the beta sheets are con-nected by a disulfide bond between a residue of a first beta sheet and a second beta sheet.

The phrase “complementarity determining region,” or the term “CDR,”includes an amino acid sequence encoded by a nucleic acid sequence of an organism's immunoglobulin genes that normally (i.e., in a wildtype animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

In an embodiment, the assignment of amino acids to each framework or CDR domain in an immunoglobulin is in accordance with the definitions of Sequences of Proteins of Immunological Interest, Kabat et al.; National Institutes of Health, Bethesda, Md.; 5th ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et al., (1989) Nature 342: 878-883. Thus, the present disclosure includes antibodies and antigen-binding fragments including the CDRs of a VH and the CDRs of a VL, which VH and VL comprise amino acid sequences as set forth herein (see e.g., sequences of Table 1-1, or a variant thereof), wherein the CDRs are as defined according to Kabat and/or Chothia.

The phrase “Fc-containing protein” includes antibodies, multispecific antibodies (e.g., bispecific antibodies), immunoadhesins, and other binding proteins that comprise at least a functional portion of an immunoglobulin CH2 and CH3 region. A “functional portion” refers to a CH2 and CH3 region that can bind a Fc receptor (e.g., an FcyR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

The term “antigen-binding protein,” as used herein, refers to a polypeptide or protein (one or more polypeptides complexed in a functional unit) that specifically recognizes an epitope on an antigen, such as a cell-specific antigen and/or a target antigen as described herein (e.g., CACNG1). An antigen-binding protein may be multispecific. The term “multispecific” with reference to an antigen-binding protein means that the protein recognizes different epitopes, either on the same antigen or on different antigens. A multispecific antigen-binding protein as described herein can be a single multifunctional polypeptide, or it can be a multimeric complex of two or more polypeptides that are covalently or non-covalently associated with one another. The term “antigen-binding protein” includes antibodies or fragments thereof as described herein that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bispecific or a multispecific antigen-binding molecule with a second binding specificity.

A CACNG1-binding protein described herein may be an antigen-binding fragment of an antibody (which optionally may be conjugated to a molecular cargo). The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments (Ward et al. (1989) Nature 241:544-546); consisting of the amino acid residues that mimic the hypervariable region of an antibody (e.g., an isolated complementarity determining region (CDR) such as a CDR3 peptide), or a constrained FR3-CDR3-FR4 peptide, and (vi) an isolated CDR. Other engineered molecules, such as domain-specific antibodies, single domain antibodies, one-armed antibodies, domain-deleted antibodies, chimeric antibodies, CDR-grafted antibodies, single chain antibodies such as diabodies (see e.g., Holliger et al. (1993) PNAS USA 90:6444-6448; Poljak et al. (1994) Structure 2:1121-1123), triabodies, tetrabodies, minibodies and small modular immunopharmaceuticals (SMIPs), are also encompassed within the expression “antigen-binding fragment,” as used herein.

An antigen-binding portion of an antibody or an antigen-binding fragment of an antibody, and the like, described herein can include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As mentioned, a CACNG1-binding protein described herein may be an scFv which may be conjugated to a molecular cargo. An scFv (single chain fragment variable) has variable regions of heavy (VH) and light (VL) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35 Å) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991, 203:46-88). In an embodiment, the linker comprises an amino acid sequence of such length to separate the variable domains by about 3.5 nm. In some embodiments, the VH and VL are connected by a linker sequence of 10 to 25 amino acids.

ScFv polypeptides may also include other amino acid sequences, such as CL or CH1 regions. ScFv molecules can be manufactured by phage display or made by directly subcloning the heavy and light chains from a hybridoma or B-cell. Ahmad et al., Clinical and Developmental Immunology, volume 2012, article ID 98025 is incorporated herein by reference for methods of making scFv fragments by phage display and antibody domain cloning.

In some embodiments, an antigen-binding protein that specifically binds to CACNG1 comprises a heavy chain variable region (HCVR or V_{H}) and/or a light chain variable region (LCVR or V_{L}).

In an embodiment, an antigen-binding protein that specifically binds to CACNG1 comprises an anti-CACNG1 scFv comprising the arrangement of variable regions as follows LCVR-HCVR or HCVR-LCVR, wherein the HCVR and LCVR are optionally connected by a linker.

In some embodiments, a CACNG1 binding protein-drug conjugate comprises a heavy chain variable region (HCVR or V_{H}) and/or a light chain variable region (LCVR or V_{L}).

In an embodiment, a CACNG1 binding protein-drug conjugate includes an anti-CACNG1 scFv comprising the arrangement of variable regions as follows LCVR-HCVR or HCVR-LCVR, wherein the HCVR and LCVR are optionally connected by a linker and the scFv is connected, optionally by a linker, to a molecular cargo (e.g., LCVR-(Gly₄Ser)₃-HCVR-molecular cargo (“GGGGSGGGGSGGGGS” disclosed as SEQ ID NO: 423); or LCVR-(Gly₄Ser)₃-HCVR-molecular cargo (“GGGGSGGGGSGGGGS” disclosed as SEQ ID NO: 423)).

The term “domain” refers to any part of a protein or polypeptide having a particular function or structure. Preferably, domains as described herein bind to cell-specific or target antigens. Cell-specific antigen or target antigen-binding domains, and the like, as used herein, include any naturally occurring, enzymatically obtainable, synthetic, or genetically engineered polypeptide or glycoprotein that specifically binds an antigen.

In some embodiments, a CACNG1 binding protein described herein comprises a half-body. The term “half-body” or “half-antibody”, which are used interchangeably, refers to half of an antibody, which essentially contains one heavy chain and one light chain. Antibody heavy chains can form dimers, thus the heavy chain of one half-body can associate with heavy chain associated with a different molecule (e.g., another half-body) or another Fc-containing polypeptide. Two slightly different Fc-domains may “heterodimerize” as in the formation of bispecific antibodies or other heterodimers, -trimers, -tetramers, and the like. See Vincent and Murini, “Current strategies in antibody engineering: Fc engineering and pH-dependent antigen binding, bispecific anti-bodies and antibody drug conjugates,” 7 Biotechnol. J. 1444-1450 (20912); and Shimamoto et al., “Peptibodies: A flexible alternative format to antibodies,” 4(5) Mabs 586-91 (2012).

In some embodiments, an anti-CACNG1 protein-drug conjugate described herein may comprise a Fab which is conjugated to a molecular cargo.

In some embodiments, an anti-CACNG1 protein-drug conjugate described herein comprise a bivalent antibody which is conjugated to a molecular cargo.

In some embodiments, a CACNG1 binding protein described herein comprises a monovalent or “one-armed” antibody. The monovalent or “one-armed” antibodies as used herein refer to immunoglobulin proteins comprising a single variable domain. For example, the one-armed antibody may comprise a single variable domain within a Fab wherein the Fab is linked to at least one Fc fragment. In certain embodiments, the one-armed antibody comprises: (i) a heavy chain comprising a heavy chain constant region and a heavy chain variable region, (ii) a light chain comprising a light chain constant region and a light chain variable region, and (iii) a polypeptide comprising a Fc fragment or a truncated heavy chain. In certain embodiments, the Fc fragment or a truncated heavy chain comprised in the separate polypeptide is a “dummy Fc” which refers to an Fc fragment that is not linked to an antigen binding domain. The one-armed antibodies of the present disclosure may comprise any of the HCVR/LCVR pairs or CDR amino acid sequences as set forth in Table 1-1 herein. One-armed antibodies comprising a full-length heavy chain, a full-length light chain and an additional Fc domain polypeptide can be constructed using standard methodologies (see, e.g., WO2010151792, which is incorporated herein by reference in its entirety), wherein the heavy chain constant region differs from the Fc domain polypeptide by at least two amino acids (e.g., H95R and Y96F according to the IMGT exon numbering system; or H435R and Y436F according to the EU numbering system). Such modifications are useful in purification of the monovalent antibodies (see WO2010151792).

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH—VH, VH—VL or VL-VL dimers. Alternatively, the antigen-binding fragment of an antibody may contain a monomeric V_{H} and/or V_{L} domain which are bound non-covalently.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) VH—CH1; (ii) VH—CH2; (iii) VH—CH3; (iv) VH-CH1-CH2; (v) VH—CH1-CH2-CH3; (vi) VH—CH2-CH3; (vii) VH—CL; (viii) VL—CH1; (ix) VL—CH2; (x) VL—CH3; (xi) VL —CH1-CH2; (xii) VL-CH1-CH2-CH3; (xiii) VL —CH2-CH3; and (xiv) VL —CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody described herein may comprise a homo-dimer or hetero-dimer (or other multimer) of any of the variable and constant domain configurations listed above in non-covalent association with one another and/or with one or more monomeric VH or VL domain (e.g., by disulfide bond(s)). The present disclosure includes an antigen-binding fragment of an antigen-binding protein such as an antibody set forth herein.

Antigen-binding proteins (e.g., antibodies and antigen-binding fragments) may be monospecific or multispecific (e.g., bispecific). Multispecific antigen-binding proteins are discussed further herein. The present disclosure includes monospecific as well as multispecific (e.g., bispecific) antigen-binding fragments comprising one or more variable domains from an antigen-binding protein that is specifically set forth herein. In some embodiments, a multispecific antigen-binding fragment of an antibody described herein can comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody as described herein using routine techniques available in the art.

An anti-CACNG1 antibody (e.g., an anti-hCACNG1 antibody) and antigen-binding fragment thereof as described herein may be monospecific or multispecific (e.g., bispecific). Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. An anti-CACNG1 antibody (e.g., an anti-hCACNG1 antibody) and antigen-binding fragment thereof as described herein can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a multispecific antibody with a second or additional binding specificity.

Use of the expression “anti-CACNG1 antibody” or “anti-hCACNG1 antibody” herein is intended to include both monospecific anti-CACNG1 antibodies, e.g., anti-hCACNG1 antibodies, as well as mutispecific antibodies, e.g., bispecific antibodies, comprising a CACNG1-binding arm and a “target”-binding arm. Thus, described herein are bispecific antibodies wherein one arm of an immunoglobulin binds CANG1, e.g., hCACNG1, and the other arm of the immunoglobulin is specific for another target molecule. The CACNG1-binding arm can comprise any of the HCVR/LCVR or CDR amino acid sequences as set forth in Table 1-1 herein.

In certain embodiments, the CACNG1-binding arm binds to CACNG1, e.g., hCACNG1, and induces internalization of the CACNG1 and antibody bound thereto. In certain embodiments, the CACNG1-binding arm binds weakly to CACNG1, e.g., hCACNG1, and induces internalization of CACNG1 and antibody bound thereto.

In certain embodiments, a bispecific antigen-binding molecule described herein is a bispecific antibody. The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 do-main, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3.

The first antigen-binding domain and the second antigen-binding domain may be directly or indirectly connected to one another to form a bispecific antigen-binding molecule as described herein. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. A “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3domain), e.g., an Fc domain of an IgG selected from the isotypes IgG1, IgG2, IgG3, and IgG4, as well as any allotype within each isotype group.

Bispecific antigen-binding molecules as described herein will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain can be an Fc fragment or an amino acid sequence of from 1 to about 200 amino acids in length containing at least one cysteine residue. In other embodiments, the multimerizing domain can be a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules as described herein. For example, an antibody or fragment thereof having a first antigen binding specificity can be functionally linked (e.g., by chemical coupling, genetic fusion, noncovalent association, or otherwise) to one or more other molecular entities, such as another antibody or antibody fragment having a second antigen-binding specificity to produce a bispecific antigen-binding molecule. Specific exemplary bispecific formats include, without limitation, e.g., scFv-based ordiabody bi-specific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats).

In the context of bispecific antigen-binding molecules as described herein, the multimerizing domains, e.g., Fc domains, may comprise one or more amino acid changes (e.g., insertions, deletions or substitutions) as compared to the wild-type, naturally occurring version of the Fc domain. For example, bispecific antigen-binding molecules may comprise one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). It is to be understood that Fc domains of antigen-binding molecules disclosed herein such as but not limited to bispecific antigen-binding molecules may comprise, without limitation, any Fc domains described herein, e.g., Fc domains comprising any of various modifications described herein.

Also described herein are bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bispecific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V821 (by IMGT; D356E, L358M, N384S, K392N, V397M, and V422I by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a hu-man IgG2 or a human IgG4 hinge region. A particular example of a chimeric Fc domain that can be included in any of the antigen-binding molecules set forth herein comprises, from N- to C-terminus: [IgG4 CH1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 CH1]-[IgG1 upper hinge]-[IgG2 lower hinge]-[IgG4 C_{H}2]-[IgG1 C_{H}3]. These and other examples of chimeric Fc domains that can be included in any of the antigen-binding molecules as described herein are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function.

The phrase “an antibody that binds CACNG1” or an “anti-CACNG1 antibody” or “anti-hCACNG1 antibody” includes an antibody and antigen-binding fragment thereof that specifically recognizes a single CACNG1 molecule. An antibody and antigen-binding fragment thereof as described herein may bind soluble CACNG1 and/or cell surface-expressed CACNG1. Soluble CACNG1 includes natural CACNG1 proteins as well as recombinant CACNG1 protein variants that lack a transmembrane domain or are otherwise unassociated with a cell membrane.

The term “specifically binds” or “binds specifically” refers to those antigen-binding proteins (e.g., antibodies or antigen-binding fragments thereof) having a binding affinity to an antigen, such as human CACNG1 protein, mouse CACNG1 protein or monkey CACNG1 protein, expressed as K_{D}, of at least about 10⁻⁹ M (e.g., 0.01, 0.1, 0.2, 0.3, 0.4, 0.5, 0.6, 0.7, 0.8, 0.9 or 1.0 nM), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present disclosure includes antigen-binding proteins that specifically bind to CACNG1 protein (e.g., CACNG1b and/or CACNG1c isoform). “Anti-CACNG1” refers to an antigen-binding protein (or other molecule), for example an antibody or antigen-binding fragment thereof, that binds specifically to CACNG1.

In some embodiments, the antigen-binding proteins (e.g., antibodies or antigen-binding fragments thereof) described herein have high affinity to CACNG1, e.g., at least 10⁻⁹ M, at least 10⁻¹⁰ M; at least 10⁻¹¹ M; or at least 10⁻¹² M, e.g., as measured by surface plasmon resonance, e.g., BIACORE™ or solution-affinity ELISA.

Cell-based binding strategies, such as fluorescent-activated cell sorting (FACS) binding assays, are also routinely used and provide binding characterization data with respect to cell-surface expressed proteins. FACS data correlates well with other methods such as radioligand competition binding and SPR (Benedict, CA, J Immunol Methods. 1997, 201(2):223-31; Geuijen, C A, et al. J Immunol Methods. 2005, 302(1-2):68-77).

Accordingly, an anti-CACNG1 antibody and antigen-binding fragment thereof as described herein bind to the predetermined antigen or cell surface molecule (receptor) having an affinity corresponding to a K_{D} value that is at least ten-fold lower than its affinity for binding to a non-specific antigen (e.g., BSA, casein). The affinity of an antibody corresponding to a K_{D} value that is equal to or less than ten-fold lower than a non-specific antigen may be considered non-detectable binding, however such an antibody may be paired with a second antigen binding arm for the production of a bispecific antibody as described herein.

The term “K_{D}” in molar (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and there-fore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio deter-mined by dividing the larger K_{D} value (lower, or weaker, affinity) by the smaller K_{D} (higher, or stronger, affinity), for example expressed as 5-fold or 10-fold greater binding affinity, as the case may be.

Also described herein is an antibody and antigen-binding fragment thereof that binds CACNG1, e.g., hCACNG1, with high, medium, or low affinity, depending on the therapeutic context and particular targeting properties that are desired. For example, in the context of a bispecific antigen-binding molecule, wherein one arm binds CACNG1 and another arm binds a target antigen (e.g., a tumor associated antigen), it may be desirable for the target antigen-binding arm to bind the target antigen with high affinity while the anti-CACNG1 arm, e.g., anti-hCACNG1, binds CACNG1 with only moderate or low affinity. In this manner, preferential targeting of the antigen-binding molecule to cells expressing the target antigen may be achieved while avoiding general/untargeted CACNG1 binding and the consequent adverse side effects associated therewith.

Also described herein are antibodies, antigen-binding fragments, and bispecific antibodies thereof that bind CACNG1, e.g., hCACNG1, with weak (i.e. low) or even no detectable affinity. In some embodiments, an antibody and antigen-binding fragment thereof as described herein can bind CACNG1, e.g., hCACNG1, (e.g., at 37° C.) with a K_{D} of greater than about 100 nM as measured by surface plasmon resonance. In some embodiments, an antibody or antigen-binding fragment as described herein binds CACNG1 with a K_{D} of greater than about greater than about 110 nM, at least 120 nM, greater than about 130 nM, greater than about 140 nM, greater than about 150 nM, at least 160 nM, greater than about 170 nM, greater than about 180 nM, greater than about 190 nM, greater than about 200 nM, greater than about 250 nM, greater than about 300 nM, greater than about 400 nM, greater than about 500 nM, greater than about 600 nM, greater than about 700 nM, greater than about 800 nM, greater than about 900 nM, or greater than about 1 μM, or with no detectable affinity, as measured by surface plasmon resonance (e.g., mAb-capture or antigen-capture format), or a substantially similar assay.

The term “k_{d}” (sec −1 or 1/s) refers to the dissociation rate constant of a particular antibody-antigen interaction, or the dissociation rate constant of an antibody or antibody-binding fragment. Said value is also referred to as the k_{off} value.

The term “kₐ” (M-1 x sec-1 or 1/M) refers to the association rate constant of a particular antibody-antigen interaction, or the association rate constant of an antibody or antibody-binding fragment.

The term “K_{A}” (M-1 or 1/M) refers to the association equilibrium constant of a particular anti-body-antigen interaction, or the association equilibrium constant of an antibody or antibody-binding fragment. The association equilibrium constant is obtained by dividing the kₐ by the k_{d}.

The term “EC₅₀” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody as described herein that gives half-maximal binding to cells expressing CACNG1, as determined by e.g. a FACS binding assay or an androgen receptor activation luciferase assay. Thus, reduced or weaker binding is observed with an increased EC₅₀ , or half maximal effective concentration value.

In one embodiment, decreased binding can be defined as an increased EC₅₀ antibody concentration which enables binding to the half-maximal amount of target cells.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

In some embodiments, an isolated antibody described herein can be an antibody that has been identified and separated and/or recovered from at least one component of its natural environment. For example, an antibody that has been separated or removed from at least one component of an organism, or from a tissue or cell in which the antibody naturally exists or is naturally produced, may be considered an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The present disclosure includes antigen-binding proteins, e.g., antibodies or antigen-binding fragments, that bind to the same epitope as an antigen-binding protein described herein.

An antigen is a molecule, such as a peptide (e.g., CACNG1 or a fragment thereof (an antigenic fragment)), to which, for example, an antibody or antigen-binding fragment thereof binds. The specific region on an antigen that an antibody recognizes and binds to is called the epitope. Antigen-binding proteins (e.g., antibodies) described herein that specifically bind to such antigens are part of the present disclosure.

The term “epitope” refers to an antigenic determinant (e.g., on CACNG1) that interacts with a specific antigen-binding site of an antigen-binding protein, e.g., a variable region of an antibody, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes to which antigen-binding proteins described herein bind may be included in fragments of CACNG1, for example the extracellular domain thereof. Antigen-binding proteins (e.g., antibodies) described herein that bind to such epitopes are part of the present disclosure.

The epitope on CACNG1 to which an anti-CACNG1 antibody, e.g., an anti-hCACNG1 antibody, and antigen-binding fragment thereof as described herein may consist of a single contiguous sequence of 3 or more (e.g., 3, 4, 5, 6, 7, 8, 9, 10, 11, 12, 13, 14, 15, 16, 17, 18, 19, 20 or more) amino acids of a CACNG1 protein. Alternatively, the epitope may consist of a plurality of non-contiguous amino acids (or amino acid sequences) of CACNG1. The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267: 252-259; Engen and Smith (2001) Anal. Chem. 73: 256A-265A. In general terms, the hydrogen/deuterium exchange method can involve deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the anti-body interacts. See, e.g., Ehring (1999) Analytical Biochemistry 267(2):252-259; Engen and Smith (2001) Anal. Chem. 73:256A-265A. X-ray crystallography of the antigen/antibody complex may also be used for epitope mapping purposes.

The present disclosure includes antigen-binding proteins that compete for binding to a CACNG1 epitope as discussed herein, with an antigen-binding protein described herein. The term “competes” as used herein, refers to an antigen-binding protein (e.g., antibody or antigen-binding fragment thereof) that binds to an antigen (e.g., CACNG1) and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between CACNG1-binding proteins (e.g., monoclonal antibodies (mAbs)) can be determined using a real time, label-free bio-layer interferometry assay on an Octet RED384 biosensor (Pall ForteBio Corp.).

One can easily determine whether a particular antigen-binding molecule (e.g., antibody) or antigen-binding domain thereof binds to the same epitope as, or competes for binding with, a reference antigen-binding molecule as described herein by using routine methods known in the art. For example, to determine if a test antibody binds to the same epitope on CACNG1 as a reference bispecific antigen-binding molecule as described herein, the reference bispecific molecule is first allowed to bind to a CACNG1 protein. Next, the ability of a test antibody to bind to the CACNG1 molecule is assessed. If the test antibody is able to bind to CACNG1 following saturation binding with the reference bispecific antigen-binding molecule, it can be concluded that the test antibody binds to a different epitope of CACNG1 than the reference bispecific antigen-binding molecule. On the other hand, if the test antibody is not able to bind to the CACNG1 molecule following saturation binding with the reference bispecific antigen-binding molecule, then the test antibody may bind to the same epitope of CACNG1 as the epitope bound by the reference bispecific antigen-binding molecule as described herein. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with some embodiments described herein, two antigen-binding proteins bind to the same (or overlapping) epitope if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino ac-id mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a CACNG1 protein under saturating conditions followed by assessment of binding of the test antibody to the CACNG1 molecule. In a second orientation, the test antibody is allowed to bind to a CACNG1 molecule under saturating conditions followed by assessment of binding of the reference antigen-binding molecule to the CACNG1 molecule. If, in both orientations, only the first (saturating) antigen-binding molecule is capable of binding to the CACNG1 molecule, then it is concluded that the test antibody and the reference antigen-binding molecule compete for binding to CACNG1. As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

Typically, an antibody or antigen-binding fragment described herein which is modified in some way retains the ability to specifically bind to CACNG1, e.g., retains at least 10% of its CACNG1 binding activity (when compared to the parental antibody) when that activity is expressed on a molar basis. Preferably, an antibody or antigen-binding fragment described herein retains at least 20%, 50%, 70%, 80%, 90%, 95% or 100% or more of the CACNG1 binding affinity as the parental antibody. It is also intended that an antibody or antigen-binding fragment described herein may include conservative or non-conservative amino acid substitutions (referred to as “conservative variants” or “function conserved variants” of the antibody) that do not substantially alter its biologic activity.

A CACNG1-binding protein described herein may be a monoclonal antibody or a CACNG1-binding fragment of a monoclonal antibody which may be conjugated to a molecular cargo. The present disclosure includes monoclonal CACNG1-binding proteins, e.g., antibodies and antigen-binding fragments thereof, as well as monoclonal compositions comprising a plurality of isolated monoclonal antigen-binding proteins. The term “monoclonal antibody” or “mAb”, as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment, a CACNG1-binding protein, e.g., antibody or antigen-binding fragment (which may be conjugated to a molecular cargo) comprises a heavy chain constant domain, e.g., of the type IgA (e.g., IgA1 or IgA2), IgD, IgE, IgG (e.g., IgG1, IgG2, IgG3 and IgG4) or IgM. In an embodiment, an antigen-binding protein, e.g., antibody or antigen-binding fragment, comprises a light chain constant domain, e.g., of the type kappa or lambda. In an embodiment, a VH as set forth herein is linked to a human heavy chain constant domain (e.g., IgG) and a VL as set forth herein is linked to a human light chain constant domain (e.g., kappa). The present disclosure includes antigen-binding proteins comprising the variable domains set forth herein, which are linked to a heavy and/or light chain constant domain, e.g., as set forth herein.

The present disclosure includes human CACNG1-binding proteins which may be conjugated to a molecular cargo. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See e.g., U.S. Pat. Nos. 8,502,018; 6,596,541 or 5,789,215. The anti-CACNG1 human mAbs described herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject. The present disclosure includes human antigen-binding proteins (e.g., antibodies or antigen-binding fragments thereof described herein).

The antibodies as described herein may, in some embodiments, be recombinant human antibodies. The term “recombinant human antibody” is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies may exist in two general forms that are associated with hinge heterogeneity. In one general form, an immunoglobulin molecule comprises a stable four chain construct of approximately 150-160 kDa in which the dimers are held together by an interchain heavy chain disulfide bond. In a second general form, the dimers are not linked via interchain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The antibodies as described herein may have one or more mutations in the hinge, C_{H}2 or C_{H}3 region which may be desirable, for example, in production, to improve the yield of the desired antibody form.

The present disclosure includes anti-CACNG1 chimeric antigen-binding proteins, e.g., antibodies and antigen-binding fragments thereof (which may be conjugated to a molecular cargo), and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567; and Morrison et al., (1984) Proc. Natl. Acad. Sci. USA 81: 6851-6855). The present disclosure includes chimeric antibodies comprising the variable domains which are set forth herein and a non-human constant domain.

The term “recombinant” CACNG1-binding proteins, such as antibodies or antigen-binding fragments thereof (which may be conjugated to a molecular cargo), refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library. The present disclosure includes recombinant antigen-binding proteins, such as antibodies and antigen-binding fragments as set forth herein.

A “variant” of a polypeptide, such as an immunoglobulin chain, refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 1-180); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment) and/or comprising the amino acid sequence but having one or more (e.g., 1, 2, 3, 4, 5, 6, 7, 8, 9 or 10) mutations (e.g., point mutation, insertion, truncation, and/or deletion).

Moreover, a variant of a polypeptide may include a polypeptide such as an immunoglobulin chain which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes CACNG1-binding proteins which include an immunoglobulin light chain (or V_{L}) variant comprising the amino acid sequence set forth in SEQ ID NO: 5, 13, 21, 29, 37, 45, 53, 61, 69, 77, 85, 93, 101, 109, 117, 125, 133, 141, 437, 459 but having one or more of such mutations and/or an immunoglobulin heavy chain (or V_{H}) variant comprising the amino acid sequence set forth in SEQ ID NO: 1, 9, 17, 25, 33, 41, 49, 57, 65, 73, 81, 89, 97, 105, 113, 121, 129, 137, 429, 451 but having one or more of such mutations. In an embodiment, a CACNG1-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships” in Atlas of Protein Sequence and Structure, (1978) vol. 5, suppl. 3.″ M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments” in Theoretical and Computational Methods in Genome Research (S. Suhai, ed.), (1997) pp. 1-14, Plenum, N.Y.

The anti-hCACNG1 antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Also described herein are antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”).

A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies as described herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, an antibody and an antigen-binding fragment that contains one or more germline mutations can be easily tested for one or more desired property such as, improved binding specificity, increased binding affinity, improved or enhanced antagonistic or agonistic biological properties (as the case may be), reduced immunogenicity, etc. In some embodiments, an antibody or an antigen-binding fragment as described herein is obtained in this general manner.

Also described herein are anti-CACNG1 antibodies comprising variants of any of the HCVR, LCVR, and/or CDR amino acid sequences disclosed herein having one or more conservative substitutions. For example, some embodiments include anti-CACNG1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1-1 herein. An antibody and antigen-binding fragment thereof as described herein may comprise one or more amino acid substitutions, insertions and/or deletions in the framework and/or CDR regions of the heavy and light chain variable domains as compared to the corresponding germline sequences from which the individual antigen-binding domains were derived while maintaining or improving the desired weak-to-no detectable binding to, e.g., CACNG1.

A “conservatively modified variant” or a “conservative substitution” or a “conservative amino acid substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. In some embodiments, a conservative amino acid substitution can maintain or improve the desired weak-to-no detectable binding affinity in the case of anti-CACNG1 binding molecules (e.g., anti-hCACNG1 binding molecules) described herein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present disclosure includes CACNG1-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Once obtained, antigen-binding domains that contain one or more germline mutations cab be tested for decreased binding affinity utilizing one or more in vitro assays. Generally antibodies that recognize a particular antigen are typically screened for their purpose by testing for high (i.e. strong) binding affinity to the antigen.

Unexpected benefits, for example, improved pharmacokinetic properties and low toxicity to the patient may be realized from further modifying the antibodies as described herein by the methods described herein.

Also described herein are anti-CACNG1 antibodies and antigen-binding fragments thereof comprising an antigen-binding domain with an HCVR and/or CDR amino acid sequence that is substantially identical to any of the HCVR and/or CDR amino acid sequences disclosed herein, while maintaining or improving the desired weak affinity to CACNG1 antigen. The term “substantial identity” or “substantially identical,” when referring to an amino acid sequence means that two amino acid sequences, when optimally aligned, such as by the programs GAP or BEST-FIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as GAP and BEST-FIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best over-lap between the query and search sequences (Pearson (2000) supra). Another preferred algorithm when comparing a sequence as described herein to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

Also described herein are anti-CACNG1 antibodies and antigen-binding fragments thereof with pH-dependent binding characteristics. For example, an anti-CACNG1 as described herein may exhibit reduced binding to CACNG1 at acidic pH as compared to neutral pH. Alternatively, anti-CACNG1 antibodies as described herein may exhibit enhanced binding to CACNG1 at acidic pH as compared to neutral pH. The expression “acidic pH” includes pH values less than about 6.2, e.g., about 6.0, 5.95, 5, 9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. The expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to CACNG1 at acidic pH as compared to neutral pH” for purposes of the description herein if the antibody or antigen-binding fragment thereof exhibits an acidic/neutral K_{D} ratio of about 3.0 or greater. In certain exemplary embodiments, the acidic/neutral K_{D} ratio for an antibody or anti-gen-binding fragment as described herein can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0. 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

Antibodies and antigen-binding fragments described herein comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to CACNG1 comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

The amino acid sequences of domains in CACNG1-binding proteins of conjugates of the present disclosure are summarized below in Table 1-1. For example, anti-CACNG1 antibodies and antigen-binding fragments thereof (e.g., scFvs and Fabs) comprising the HCVR and LCVR of the molecules in Table 1-1; or comprising the CDRs thereof, conjugated to a molecular cargo, form part of the present disclosure.

TABLE 1-1
SEQ ID NOs or Sequences of Amino Acid Sequences of Domains in Antibodies or Antigen-binding Fragments
(e.g., Fabs or scFv Molecules) in Protein-Drug Conjugates of the Present Disclosure.

| | anti-CACNG1 | | | | | | | | | | |
|---|---|---|---|---|---|---|---|---|---|---|---|
| # | Molecule | HCVR | HCDR1 | HCDR2 | HCDR3 | LCVR | LCDR1 | LCDR2 | LCDR3 | HC | LC |
| 1 | H2aM31929N/ | 1 | 2 | 3 | 4 | 5 | 6 | AAS | 8 | 145 | 146 |
| | REGN10728 | | | | | | | | | | |
| 2 | H2aM31944N | 9 | 10 | 11 | 12 | 13 | 14 | AAS | 16 | 147 | 148 |
| 3 | H4H31265P2/ | 17 | 18 | 19 | 20 | 21 | 22 | 23 | 24 | 149 | 150 |
| | REGN5972 | | | | | | | | | | |
| 4 | H2aM31941N | 25 | 26 | 27 | 28 | 29 | 30 | 31 | 32 | 151 | 152 |
| 5 | REGN7660 | 33 | 34 | 35 | 36 | 37 | 38 | GAS | 40 | 153 | 154 |
| 6 | REGN9909 | 41 | 42 | 43 | 44 | 45 | 46 | RNN | 48 | 155 | 156 |
| 7 | REGN10713 | 49 | 50 | 51 | 52 | 53 | 54 | DNN | 56 | 157 | 158 |
| 8 | REGN10715 | 57 | 58 | 59 | 60 | 61 | 62 | ATS | 64 | 159 | 160 |
| 9 | REGN10716 | 65 | 66 | 67 | 68 | 69 | 70 | GAS | 72 | 161 | 162 |
| 10 | REGN10717 | 73 | 74 | 75 | 76 | 77 | 78 | KAS | 80 | 163 | 164 |
| 11 | REGN10783 | 81 | 82 | 83 | 84 | 85 | 86 | AAS | 88 | 165 | 166 |
| 12 | REGN7854 | 89 | 90 | 91 | 92 | 93 | 94 | GAS | 96 | 167 | 168 |
| 13 | REGN14570 | 97 | 98 | 99 | 100 | 101 | 102 | ATS | 104 | 169 | 170 |
| 14 | REGN14571 | 105 | 106 | 107 | 108 | 109 | 110 | GAS | 112 | 171 | 172 |
| 15 | REGN14572 | 113 | 114 | 115 | 116 | 117 | 118 | KAS | 120 | 173 | 174 |
| 16 | REGN14573 | 121 | 122 | 123 | 124 | 125 | 126 | DNN | 128 | 175 | 176 |
| 17 | REGN14574 | 129 | 130 | 131 | 132 | 133 | 134 | AAS | 136 | 177 | 178 |
| 18 | REGN14647 | 137 | 138 | 139 | 140 | 141 | 142 | AAS | 144 | 179 | 180 |
| 19 | REGN10712 | 429 | 431 | 433 | 435 | 437 | 439 | YNS | 441 | 443 | 447 |
| 20 | REGN9908 | 451 | 453 | 455 | 457 | 459 | 461 | RNN | 463 | 465 | 469 |

Sequences of domains in antibodies or antigen-binding fragments (e.g., Fabs or scFv molecules) in protein-drug conjugates described herein are set forth below.

H2aM31929N/REGN10728
HCVR DNA Sequence
(SEQ ID NO: 181)
CAGGTGCAGCTGGTGGAGTCTGGGGGAGGCGTGGTCCAGCCTGGGAG
GTCCCTGAGACTCTCCTGTACAGCGTCTGGAATCACCTTCAGAAATT
ATGGCATGCACTGGGTCCGCCAGGCTCCAGGCAAGGGGCTGGAGTGG
GTGGCAGTTATGTGGTATGATGGAAGTAATAAGTACTATGCAGACTC
CGTGAAGGGCCGTTTCACCATCTCCGGAGACAATTCCAAGGTGTATC
TGCAAATGAACAGCCTGAGAGCCGAGGACACGGCTGTATATTACTGT
GCGAGAAGGGGCACTATAAGAACAGCTGCCCCTTTTGACTACTGGGG
TCAGGGAACCCTGGTCACCGTCTCCTCA
HCVR Amino Acid Sequence
(SEQ ID NO: 1)
QVQLVESGGGVVQPGRSLRLSCTASGITFRNYGMHWVRQAPGKGLEW
VAVMWYDGSNKYYADSVKGRFTISGDNSKVYLQMNSLRAEDTAVYYC
ARRGTIRTAAPFDYWGQGTLVTVSS
HCDR1 DNA Sequence
(SEQ ID NO: 182)
GGAATCACCTTCAGAAATTATGGC
HCDR1 Amino Acid Sequence
(SEQ ID NO: 2)
GITFRNYG
HCDR2 DNA Sequence
(SEQ ID NO: 183)
ATGTGGTATGATGGAAGTAATAAG
HCDR2 Amino Acid Sequence
(SEQ ID NO: 3)
MWYDGSNK
HCDR3 DNA Sequence
(SEQ ID NO: 184)
GCGAGAAGGGGCACTATAAGAACAGCTGCCCCTTTTGACTAC
HCDR3 Amino Acid Sequence
(SEQ ID NO: 4)
ARRGTIRTAAPFDY
LCVR DNA Sequence
(SEQ ID NO: 185)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAA
LCVR Amino Acid Sequence
(SEQ ID NO: 5)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIK
LCDR1 DNA Sequence
(SEQ ID NO: 186)
CAGAGCATTAGCAGCTAT
LCDR1 Amino Acid Sequence
(SEQ ID NO: 6)
QSISSY
LCDR2 DNA Sequence
(SEQ ID NO: 187)
GCTGCATCC
LCDR2 Amino Acid Sequence
(SEQ ID NO: 7)
AAS
LCDR3 DNA Sequence
(SEQ ID NO: 188)
CAACAGAGTTACAGTACCCCTCCGATCACC
LCDR3 Amino Acid Sequence
(SEQ ID NO: 8)
QQSYSTPPIT
HC DNA Sequence
(SEQ ID NO: 189)
CAGGTGCAGCTGGTGGAGTCTGGGGGAGGCGTGGTCCAGCCTGGGAG
GTCCCTGAGACTCTCCTGTACAGCGTCTGGAATCACCTTCAGAAATT
ATGGCATGCACTGGGTCCGCCAGGCTCCAGGCAAGGGGCTGGAGTGG
GTGGCAGTTATGTGGTATGATGGAAGTAATAAGTACTATGCAGACTC
CGTGAAGGGCCGTTTCACCATCTCCGGAGACAATTCCAAGGTGTATC
TGCAAATGAACAGCCTGAGAGCCGAGGACACGGCTGTATATTACTGT
GCGAGAAGGGGCACTATAAGAACAGCTGCCCCTTTTGACTACTGGGG
TCAGGGAACCCTGGTCACCGTCTCCTCAGCCTCCACCAAGGGCCCAT
CGGTCTTCCCCCTGGCGCCCTGCTCCAGGAGCACCTCCGAGAGCACA
GCCGCCCTGGGCTGCCTGGTCAAGGACTACTTCCCCGAACCGGTGAC
GGTGTCGTGGAACTCAGGCGCCCTGACCAGCGGCGTGCACACCTTCC
CGGCTGTCCTACAGTCCTCAGGACTCTACTCCCTCAGCAGCGTGGTG
ACCGTGCCCTCCAGCAGCTTGGGCACGAAGACCTACACCTGCAACGT
AGATCACAAGCCCAGCAACACCAAGGTGGACAAGAGAGTTGAGTCCA
AATATGGTCCCCCATGCCCACCGTGCCCAGCACCAGGCGGTGGCGGA
CCATCAGTCTTCCTGTTCCCCCCAAAACCCAAGGACACTCTCATGAT
CTCCCGGACCCCTGAGGTCACGTGCGTGGTGGTGGACGTGAGCCAGG
AAGACCCCGAGGTCCAGTTCAACTGGTACGTGGATGGCGTGGAGGTG
CATAATGCCAAGACAAAGCCGCGGGAGGAGCAGTTCAACAGCACGTA
CCGTGTGGTCAGCGTCCTCACCGTCCTGCACCAGGACTGGCTGAACG
GCAAGGAGTACAAGTGCAAGGTCTCCAACAAAGGCCTCCCGTCCTCC
ATCGAGAAAACCATCTCCAAAGCCAAAGGGCAGCCCCGAGAGCCACA
GGTGTACACCCTGCCCCCATCCCAGGAGGAGATGACCAAGAACCAGG
TCAGCCTGACCTGCCTGGTCAAAGGCTTCTACCCCAGCGACATCGCC
GTGGAGTGGGAGAGCAATGGGCAGCCGGAGAACAACTACAAGACCAC
GCCTCCCGTGCTGGACTCCGACGGCTCCTTCTTCCTCTACAGCAGGC
TCACCGTGGACAAGAGCAGGTGGCAGGAGGGGAATGTCTTCTCATGC
TCCGTGATGCATGAGGCTCTGCACAACCACTACACACAGAAGTCCCT
CTCCCTGTCTCTGGGTAAATGA
HC Amino Acid Sequence
(SEQ ID NO: 145)
QVQLVESGGGVVQPGRSLRLSCTASGITFRNYGMHWVRQAPGKGLEW
VAVMWYDGSNKYYADSVKGRFTISGDNSKVYLQMNSLRAEDTAVYYC
ARRGTIRTAAPFDYWGQGTLVTVSSASTKGPSVFPLAPCSRSTSEST
AALGCLVKDYFPEPVTVSWNSGALTSGVHTFPAVLQSSGLYSLSSVV
TVPSSSLGTKTYTCNVDHKPSNTKVDKRVESKYGPPCPPCPAPGGGG
PSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSS
IEKTISKAKGQPREPQVYTLPPSQEEMTKNQVSLTCLVKGFYPSDIA
VEWESNGQPENNYKTTPPVLDSDGSFFLYSRLTVDKSRWQEGNVFSC
SVMHEALHNHYTQKSLSLSLGK
LC DNA Sequence
(SEQ ID NO: 190)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 146)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC
H2aM31944N
HCVR DNA Sequence
(SEQ ID NO: 191)
CAGGTGCAGTTGGTGGAGTCTGGGGGAGGCGTGGTCCAGCCTGGGAG
GTCCCTGAGACTCTCCTGTGAAGCGTCTGGAATCACCTTCAGAAACT
ATGGCATGCACTGGGTCCGCCAGGCTCCAGGCAAGGGGCTGGAGTGG
GTGGCAGTTATGTGGTATGATGGAAGTAATAAATACTACGCAGACTC
CGTGAAGGGCCGATTCACCATCTCCAGAGACAATTCCAAGAACACGG
TGTATCTGCAAATGAACAGCCTGAGAGCCGAAGACACGGCTGTGTAT
TACTGTGCGAGACGGGGTCATATAGCAACAGCTGCTCCCTTTGACTA
CTGGGGCCAGGGAACCCTGGTCACCGTCTCCTCA
HCVR Amino Acid Sequence
(SEQ ID NO: 9)
QVQLVESGGGVVQPGRSLRLSCEASGITFRNYGMHWVRQAPGKGLEW
VAVMWYDGSNKYYADSVKGRFTISRDNSKNTVYLQMNSLRAEDTAVY
YCARRGHIATAAPFDYWGQGTLVTVSS
HCDR1 DNA Sequence
(SEQ ID NO: 192)
GGAATCACCTTCAGAAACTATGGC
HCDR1 Amino Acid Sequence
(SEQ ID NO: 10)
GITFRNYG
HCDR2 DNA Sequence
(SEQ ID NO: 193)
ATGTGGTATGATGGAAGTAATAAA
HCDR2 Amino Acid Sequence
(SEQ ID NO: 11)
MWYDGSNK
HCDR3 DNA Sequence
(SEQ ID NO: 194)
GCGAGACGGGGTCATATAGCAACAGCTGCTCCCTTTGACTAC
HCDR3 Amino Acid Sequence
(SEQ ID NO: 12)
ARRGHIATAAPFDY
LCVR DNA Sequence
(SEQ ID NO: 195)
GACATCCAGATGACCCAGTCTCCATCCTCCCTGTCTGCATCCGTAGG
AGACAGAGTCACCATCAGTTGCCGGGCAAGTCAGAGCATTAGTAGTT
ATTTAAATTGGTATCAGCAGAAACCAGGGAAAGCCCCTAAGGTCCTG
ATGTATGCTGCATCCAGTTTGCAAAGTGGGGTCCCATCAAGGTTCAG
TGGCAGTGGATCTGGGACAGATTTCACTCTCACCATCAGCAGTCTGC
AACCTGAGGATTTTGCAACTTACTACTGTCAACAGAGTTACAGTACC
CCTCCGATCACCTTCGGCCAAGGGACACGACTGGAGATTAAA
LCVR Amino Acid Sequence
(SEQ ID NO: 13)
DIQMTQSPSSLSASVGDRVTISCRASQSISSYLNWYQQKPGKAPKVL
MYAASSLQSGVPSRFSGSGSGTDFTLTISSLQPEDFATYYCQQSYST
PPITFGQGTRLEIK
LCDR1 DNA Sequence
(SEQ ID NO: 196)
CAGAGCATTAGTAGTTAT
LCDR1 Amino Acid Sequence
(SEQ ID NO: 14)
QSISSY
LCDR2 DNA Sequence
(SEQ ID NO: 197)
GCTGCATCC
LCDR2 Amino Acid Sequence
(SEQ ID NO: 15)
AAS
LCDR3 DNA Sequence
(SEQ ID NO: 198)
CAACAGAGTTACAGTACCCCTCCGATCACC
LCDR3 Amino Acid Sequence
(SEQ ID NO: 16)
QQSYSTPPIT
HC DNA Sequence
(SEQ ID NO: 199)
CAGGTGCAGTTGGTGGAGTCTGGGGGAGGCGTGGTCCAGCCTGGGAG
GTCCCTGAGACTCTCCTGTGAAGCGTCTGGAATCACCTTCAGAAACT
ATGGCATGCACTGGGTCCGCCAGGCTCCAGGCAAGGGGCTGGAGTGG
GTGGCAGTTATGTGGTATGATGGAAGTAATAAATACTACGCAGACTC
CGTGAAGGGCCGATTCACCATCTCCAGAGACAATTCCAAGAACACGG
TGTATCTGCAAATGAACAGCCTGAGAGCCGAAGACACGGCTGTGTAT
TACTGTGCGAGACGGGGTCATATAGCAACAGCTGCTCCCTTTGACTA
CTGGGGCCAGGGAACCCTGGTCACCGTCTCCTCAGCCAAAACAACAG
CCCCATCGGTCTATCCACTGGCCCCTGTGTGTGGAGATACAACTGGC
TCCTCGGTGACTCTAGGATGCCTGGTCAAGGGTTATTTCCCTGAGCC
AGTGACCTTGACCTGGAACTCTGGATCCCTGTCCAGTGGTGTGCACA
CCTTCCCAGCTGTCCTGCAGTCTGACCTCTACACCCTCAGCAGCTCA
GTGACTGTAACCTCGAGCACCTGGCCCAGCCAGTCCATCACCTGCAA
TGTGGCCCACCCGGCAAGCAGCACCAAGGTGGACAAGAAAATTGAGC
CCAGAGGGCCCACAATCAAGCCCTGTCCTCCATGCAAATGCCCAGCA
CCTAACCTCTTGGGTGGACCATCCGTCTTCATCTTCCCTCCAAAGAT
CAAGGATGTACTCATGATCTCCCTGAGCCCCATAGTCACATGTGTGG
TGGTGGATGTGAGCGAGGATGACCCAGATGTCCAGATCAGCTGGTTT
GTGAACAACGTGGAAGTACACACAGCTCAGACACAAACCCATAGAGA
GGATTACAACAGTACTCTCCGGGTGGTCAGTGCCCTCCCCATCCAGC
ACCAGGACTGGATGAGTGGCAAGGAGTTCAAATGCAAGGTCAACAAC
AAAGACCTCCCAGCGCCCATCGAGAGAACCATCTCAAAACCCAAAGG
GTCAGTAAGAGCTCCACAGGTATATGTCTTGCCTCCACCAGAAGAAG
AGATGACTAAGAAACAGGTCACTCTGACCTGCATGGTCACAGACTTC
ATGCCTGAAGACATTTACGTGGAGTGGACCAACAACGGGAAAACAGA
GCTAAACTACAAGAACACTGAACCAGTCCTGGACTCTGATGGTTCTT
ACTTCATGTACAGCAAGCTGAGAGTGGAAAAGAAGAACTGGGTGGAA
AGAAATAGCTACTCCTGTTCAGTGGTCCACGAGGGTCTGCACAATCA
CCACACGACTAAGAGCTTCTCCCGGACTCCGGGTAAATGA
HC Amino Acid Sequence
(SEQ ID NO: 147)
QVQLVESGGGVVQPGRSLRLSCEASGITFRNYGMHWVRQAPGKGLEW
VAVMWYDGSNKYYADSVKGRFTISRDNSKNTVYLQMNSLRAEDTAVY
YCARRGHIATAAPFDYWGQGTLVTVSSAKTTAPSVYPLAPVCGDTTG
SSVTLGCLVKGYFPEPVTLTWNSGSLSSGVHTFPAVLQSDLYTLSSS
VTVTSSTWPSQSITCNVAHPASSTKVDKKIEPRGPTIKPCPPCKCPA
PNLLGGPSVFIFPPKIKDVLMISLSPIVTCVVVDVSEDDPDVQISWF
VNNVEVHTAQTQTHREDYNSTLRVVSALPIQHQDWMSGKEFKCKVNN
KDLPAPIERTISKPKGSVRAPQVYVLPPPEEEMTKKQVTLTCMVTDF
MPEDIYVEWTNNGKTELNYKNTEPVLDSDGSYFMYSKLRVEKKNWVE
RNSYSCSVVHEGLHNHHTTKSFSRTPGK
LC DNA Sequence
(SEQ ID NO: 200)
GACATCCAGATGACCCAGTCTCCATCCTCCCTGTCTGCATCCGTAGG
AGACAGAGTCACCATCAGTTGCCGGGCAAGTCAGAGCATTAGTAGTT
ATTTAAATTGGTATCAGCAGAAACCAGGGAAAGCCCCTAAGGTCCTG
ATGTATGCTGCATCCAGTTTGCAAAGTGGGGTCCCATCAAGGTTCAG
TGGCAGTGGATCTGGGACAGATTTCACTCTCACCATCAGCAGTCTGC
AACCTGAGGATTTTGCAACTTACTACTGTCAACAGAGTTACAGTACC
CCTCCGATCACCTTCGGCCAAGGGACACGACTGGAGATTAAACGAGC
TGATGCTGCACCAACTGTATCCATCTTCCCACCATCCAGTGAGCAGT
TAACATCTGGAGGTGCCTCAGTCGTGTGCTTCTTGAACAACTTCTAC
CCCAAAGACATCAATGTCAAGTGGAAGATTGATGGCAGTGAACGACA
AAATGGCGTCCTGAACAGTTGGACTGATCAGGACAGCAAAGACAGCA
CCTACAGCATGAGCAGCACCCTCACGTTGACCAAGGACGAGTATGAA
CGACATAACAGCTATACCTGTGAGGCCACTCACAAGACATCAACTTC
ACCCATTGTCAAGAGCTTCAACAGGGGAGAGTGTTGA
LC Amino Acid Sequence
(SEQ ID NO: 148)
DIQMTQSPSSLSASVGDRVTISCRASQSISSYLNWYQQKPGKAPKVL
MYAASSLQSGVPSRFSGSGSGTDFTLTISSLOPEDFATYYCQQSYST
PPITFGQGTRLEIKRADAAPTVSIFPPSSEQLTSGGASVVCFLNNFY
PKDINVKWKIDGSERQNGVLNSWTDQDSKDSTYSMSSTLTLTKDEYE
RHNSYTCEATHKTSTSPIVKSFNRGEC
H4H31265P2/REGN5972
HCVR DNA Sequence
(SEQ ID NO: 201)
CAGGTGCAGCTGGTGGAGTCTGGGGGAGGCGTGGTCCAGCCTGGGAG
GTCCCTGAGACTCTCCTGTACAGCGTCTGGATTCACCTTCCGTTCCT
ATGGCATGCACTGGGTCCGCCAGGCTCCAGGCAAGGGGCTGGAGTGG
GTGTCAGTTATTTGGATTGATGGAAATAATATATACTATGCAGACTC
CGTGAAGGGCCGATTCACCATCTCCAGAGACAATTCCAAGAACACGC
TGTATCTGCAAATGGACAGCCTGAGAGCCGAGGACACGGCTGTTTAT
TACTGTGCGAGAAGACTGGCTATAACATCAGCTGCCCCCTTTGACTA
CTGGGGCCAGGGAACCCTGGTCACCGTCTCCTCA
HCVR Amino Acid Sequence
(SEQ ID NO: 17)
QVQLVESGGGVVQPGRSLRLSCTASGFTFRSYGMHWVRQAPGKGLEW
VSVIWIDGNNIYYADSVKGRFTISRDNSKNTLYLQMDSLRAEDTAVY
YCARRLAITSAAPFDYWGQGTLVTVSS
HCDR1 DNA Sequence
(SEQ ID NO: 202)
GGATTCACCTTCCGTTCCTATGGC
HCDR1 Amino Acid Sequence
(SEQ ID NO: 18)
GFTFRSYG
HCDR2 DNA Sequence
(SEQ ID NO: 203)
ATTTGGATTGATGGAAATAATATA
HCDR2 Amino Acid Sequence
(SEQ ID NO: 19)
IWIDGNNI
HCDR3 DNA Sequence
(SEQ ID NO: 204)
GCGAGAAGACTGGCTATAACATCAGCTGCCCCCTTTGACTAC
HCDR3 Amino Acid Sequence
(SEQ ID NO: 20)
ARRLAITSAAPFDY
LCVR DNA Sequence
(SEQ ID NO: 205)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAA
LCVR Amino Acid Sequence
(SEQ ID NO: 21)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIK
LCDR1 DNA Sequence
(SEQ ID NO: 206)
CAGAGCATTAGCAGCTAT
LCDR1 Amino Acid Sequence
(SEQ ID NO: 22)
QSISSY
LCDR2 DNA Sequence
(SEQ ID NO: 207)
GCTGCATCC
LCDR2 Amino Acid Sequence
(SEQ ID NO: 23)
AAS
LCDR3 DNA Sequence
(SEQ ID NO: 208)
CAACAGAGTTACAGTACCCCTCCGATCACC
LCDR3 Amino Acid Sequence
(SEQ ID NO: 24)
QQSYSTPPIT
HC DNA Sequence
(SEQ ID NO: 209)
CAGGTGCAGCTGGTGGAGTCTGGGGGAGGCGTGGTCCAGCCTGGGAG
GTCCCTGAGACTCTCCTGTACAGCGTCTGGATTCACCTTCCGTTCCT
ATGGCATGCACTGGGTCCGCCAGGCTCCAGGCAAGGGGCTGGAGTGG
GTGTCAGTTATTTGGATTGATGGAAATAATATATACTATGCAGACTC
CGTGAAGGGCCGATTCACCATCTCCAGAGACAATTCCAAGAACACGC
TGTATCTGCAAATGGACAGCCTGAGAGCCGAGGACACGGCTGTTTAT
TACTGTGCGAGAAGACTGGCTATAACATCAGCTGCCCCCTTTGACTA
CTGGGGCCAGGGAACCCTGGTCACCGTCTCCTCAGCCTCCACCAAGG
GCCCATCGGTCTTCCCCCTGGCGCCCTGCTCCAGGAGCACCTCCGAG
AGCACAGCCGCCCTGGGCTGCCTGGTCAAGGACTACTTCCCCGAACC
GGTGACGGTGTCGTGGAACTCAGGCGCCCTGACCAGCGGCGTGCACA
CCTTCCCGGCTGTCCTACAGTCCTCAGGACTCTACTCCCTCAGCAGC
GTGGTGACCGTGCCCTCCAGCAGCTTGGGCACGAAGACCTACACCTG
CAACGTAGATCACAAGCCCAGCAACACCAAGGTGGACAAGAGAGTTG
AGTCCAAATATGGTCCCCCATGCCCACCCTGCCCAGCACCTGAGTTC
CTGGGGGGACCATCAGTCTTCCTGTTCCCCCCAAAACCCAAGGACAC
TCTCATGATCTCCCGGACCCCTGAGGTCACGTGCGTGGTGGTGGACG
TGAGCCAGGAAGACCCCGAGGTCCAGTTCAACTGGTACGTGGATGGC
GTGGAGGTGCATAATGCCAAGACAAAGCCGCGGGAGGAGCAGTTCAA
CAGCACGTACCGTGTGGTCAGCGTCCTCACCGTCCTGCACCAGGACT
GGCTGAACGGCAAGGAGTACAAGTGCAAGGTCTCCAACAAAGGCCTC
CCGTCCTCCATCGAGAAAACCATCTCCAAAGCCAAAGGGCAGCCCCG
AGAGCCACAGGTGTACACCCTGCCCCCATCCCAGGAGGAGATGACCA
AGAACCAGGTCAGCCTGACCTGCCTGGTCAAAGGCTTCTACCCCAGC
GACATCGCCGTGGAGTGGGAGAGCAATGGGCAGCCGGAGAACAACTA
CAAGACCACGCCTCCCGTGCTGGACTCCGACGGCTCCTTCTTCCTCT
ACAGCAGGCTCACCGTGGACAAGAGCAGGTGGCAGGAGGGGAATGTC
TTCTCATGCTCCGTGATGCATGAGGCTCTGCACAACCACTACACACA
GAAGTCCCTCTCCCTGTCTCTGGGTAAATGA
HC Amino Acid Sequence
(SEQ ID NO: 149)
QVQLVESGGGVVQPGRSLRLSCTASGFTFRSYGMHWVRQAPGKGLEW
VSVIWIDGNNIYYADSVKGRFTISRDNSKNTLYLQMDSLRAEDTAVY
YCARRLAITSAAPFDYWGQGTLVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPS
DIAVEWESNGQPENNYKTTPPVLDSDGSFFLYSRLTVDKSRWQEGNV
FSCSVMHEALHNHYTQKSLSLSLGK*
LC DNA Sequence
(SEQ ID NO: 210)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 150)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC*
H2aM31941N
HCVR DNA Sequence
(SEQ ID NO: 211)
CAGGTTCAGCTGGTGCAGTCTGGAGCTGAGGTGAAGAAGCCTGGGGC
CTCAGTGAAGGTCTCCTGCAAGGCTTCTGGTTACGCCTTCACCACCT
ATGGTATCACCTGGGTGCGACAGGCCCCTGGACAAGGACTTGAGTGG
ATGGGATGGATCAGCGCTTACAATGGAAATACAAACTATGCAGAGAA
GGTCCAGGGCAGATTCACCATGACCACAGACACATCCACGAATACAG
CCTACATGGAGCTGAGGAGCCTGAGATCCGACGACACGGCCGTGTAT
TTCTGTGCGAGAAAGGGTCACTATGGTTCGGGGACTTATTATAACCC
CTTTGGTTTTGATTTTTGGGGCCAAGGGACAATGGTCACCGTCTCTT
CA
HCVR Amino Acid Sequence
(SEQ ID NO: 25)
QVQLVQSGAEVKKPGASVKVSCKASGYAFTTYGITWVRQAPGQGLEW
MGWISAYNGNTNYAEKVQGRFTMTTDTSTNTAYMELRSLRSDDTAVY
FCARKGHYGSGTYYNPFGFDFWGQGTMVTVSS
HCDR1 DNA Sequence
(SEQ ID NO: 212)
GGTTACGCCTTCACCACCTATGGT
HCDR1 Amino Acid Sequence
(SEQ ID NO: 26)
GYAFTTYG
HCDR2 DNA Sequence
(SEQ ID NO: 213)
ATCAGCGCTTACAATGGAAATACA
HCDR2 Amino Acid Sequence
(SEQ ID NO: 27)
ISAYNGNT
HCDR3 DNA Sequence
(SEQ ID NO: 214)
GCGAGAAAGGGTCACTATGGTTCGGGGACTTATTATAACCCCTTTGG
TTTTGATTTT
HCDR3 Amino Acid Sequence
(SEQ ID NO: 28)
ARKGHYGSGTYYNPFGFDF
LCVR DNA Sequence
(SEQ ID NO: 215)
GAAATTATGTTGATGCAGTCTCCAGGCACCCTGTCTTTGTCTCCAGG
GGAAAGAGCCACCCTCTCCTGCAGGGCCAGTCAGAGTGTTAGCAGCA
GCTACTTAGCCTGGTACCAACAGAAACCTGGCCAGGCTCCCAGGCTC
CTCATCTATGGTGCATCCAGCAGGGCCACTGACATCCCAGACAGGTT
CAGTGGCAGTGGGTCTGGGACAGACTTCACTCTCACCATCAGCAGAC
TGGAGCCTGAAGATTTTGCAGTTTATTTCTGTCAGCAGTATTATGGC
TCACCTTGGACGTTCGGCCAAGGGACCAAGGTGGAAATCAAGCG
LCVR Amino Acid Sequence
(SEQ ID NO: 29)
EIMLMQSPGTLSLSPGERATLSCRASQSVSSSYLAWYQQKPGQAPRL
LIYGASSRATDIPDRFSGSGSGTDFTLTISRLEPEDFAVYFCQQYYG
SPWTFGQGTKVEIK
LCDR1 DNA Sequence
(SEQ ID NO: 216)
CAGAGTGTTAGCAGCAGCTAC
LCDR1 Amino Acid Sequence
(SEQ ID NO: 30)
QSVSSSY
LCDR2 DNA Sequence
(SEQ ID NO: 217)
GGTGCATCC
LCDR2 Amino Acid Sequence
(SEQ ID NO: 31)
GAS
LCDR3 DNA Sequence
(SEQ ID NO: 218)
CAGCAGTATTATGGCTCACCTTGGACG
LCDR3 Amino Acid Sequence
(SEQ ID NO: 32)
QQYYGSPWT
HC DNA Sequence
(SEQ ID NO: 219)
CAGGTTCAGCTGGTGCAGTCTGGAGCTGAGGTGAAGAAGCCTGGGGC
CTCAGTGAAGGTCTCCTGCAAGGCTTCTGGTTACGCCTTCACCACCT
ATGGTATCACCTGGGTGCGACAGGCCCCTGGACAAGGACTTGAGTGG
ATGGGATGGATCAGCGCTTACAATGGAAATACAAACTATGCAGAGAA
GGTCCAGGGCAGATTCACCATGACCACAGACACATCCACGAATACAG
CCTACATGGAGCTGAGGAGCCTGAGATCCGACGACACGGCCGTGTAT
TTCTGTGCGAGAAAGGGTCACTATGGTTCGGGGACTTATTATAACCC
CTTTGGTTTTGATTTTTGGGGCCAAGGGACAATGGTCACCGTCTCTT
CAGCCAAAACGACACCCCCATCTGTCTATCCACTGGCCCCTGGATCT
GCTGCCCAAACTAACTCCATGGTGACCCTGGGATGCCTGGTCAAGGG
CTATTTCCCTGAGCCAGTGACAGTGACCTGGAACTCTGGATCCCTGT
CCAGCGGTGTGCACACCTTCCCAGCTGTCCTGCAGTCTGACCTCTAC
ACTCTGAGCAGCTCAGTGACTGTCCCCTCCAGCACCTGGCCCAGCGA
GACCGTCACCTGCAACGTTGCCCACCCGGCCAGCAGCACCAAGGTGG
ACAAGAAAATTGTGCCCAGGGATTGTGGTTGTAAGCCTTGCATATGT
ACAGTCCCAGAAGTATCATCTGTCTTCATCTTCCCCCCAAAGCCCAA
GGATGTGCTCACCATTACTCTGACTCCTAAGGTCACGTGTGTTGTGG
TAGACATCAGCAAGGATGATCCCGAGGTCCAGTTCAGCTGGTTTGTA
GATGATGTGGAGGTGCACACAGCTCAGACGCAACCCCGGGAGGAGCA
GTTCAACAGCACTTTCCGCTCAGTCAGTGAACTTCCCATCATGCACC
AGGACTGGCTCAATGGCAAGGAGTTCAAATGCAGGGTCAACAGTGCA
GCTTTCCCTGCCCCCATCGAGAAAACCATCTCCAAAACCAAAGGCAG
ACCGAAGGCTCCACAGGTGTACACCATTCCACCTCCCAAGGAGCAGA
TGGCCAAGGATAAAGTCAGTCTGACCTGCATGATAACAGACTTCTTC
CCTGAAGACATTACTGTGGAGTGGCAGTGGAATGGGCAGCCAGCGGA
GAACTACAAGAACACTCAGCCCATCATGGACACAGATGGCTCTTACT
TCGTCTACAGCAAGCTCAATGTGCAGAAGTCCAACTGGGAGGCAGGA
AATACTTTCACCTGCTCTGTGTTACATGAGGGCCTGCACAACCACCA
TACTGAGAAGTCCCTCTCCCACTCTCCTGGTAAATGA
HC Amino Acid Sequence
(SEQ ID NO: 151)
QVQLVQSGAEVKKPGASVKVSCKASGYAFTTYGITWVRQAPGQGLEW
MGWISAYNGNTNYAEKVQGRFTMTTDTSTNTAYMELRSLRSDDTAVY
FCARKGHYGSGTYYNPFGFDFWGQGTMVTVSSAKTTPPSVYPLAPGS
AAQTNSMVTLGCLVKGYFPEPVTVTWNSGSLSSGVHTFPAVLQSDLY
TLSSSVTVPSSTWPSETVTCNVAHPASSTKVDKKIVPRDCGCKPCIC
TVPEVSSVFIFPPKPKDVLTITLTPKVTCVVVDISKDDPEVQFSWFV
DDVEVHTAQTQPREEQFNSTFRSVSELPIMHQDWLNGKEFKCRVNSA
AFPAPIEKTISKTKGRPKAPQVYTIPPPKEQMAKDKVSLTCMITDFF
PEDITVEWQWNGQPAENYKNTQPIMDTDGSYFVYSKLNVQKSNWEAG
NTFTCSVLHEGLHNHHTEKSLSHSPGK
LC DNA Sequence
(SEQ ID NO: 220)
GAAATTATGTTGATGCAGTCTCCAGGCACCCTGTCTTTGTCTCCAGG
GGAAAGAGCCACCCTCTCCTGCAGGGCCAGTCAGAGTGTTAGCAGCA
GCTACTTAGCCTGGTACCAACAGAAACCTGGCCAGGCTCCCAGGCTC
CTCATCTATGGTGCATCCAGCAGGGCCACTGACATCCCAGACAGGTT
CAGTGGCAGTGGGTCTGGGACAGACTTCACTCTCACCATCAGCAGAC
TGGAGCCTGAAGATTTTGCAGTTTATTTCTGTCAGCAGTATTATGGC
TCACCTTGGACGTTCGGCCAAGGGACCAAGGTGGAAATCAAGCGAGC
TGATGCTGCACCAACTGTATCCATCTTCCCACCATCCAGTGAGCAGT
TAACATCTGGAGGTGCCTCAGTCGTGTGCTTCTTGAACAACTTCTAC
CCCAAAGACATCAATGTCAAGTGGAAGATTGATGGCAGTGAACGACA
AAATGGCGTCCTGAACAGTTGGACTGATCAGGACAGCAAAGACAGCA
CCTACAGCATGAGCAGCACCCTCACGTTGACCAAGGACGAGTATGAA
CGACATAACAGCTATACCTGTGAGGCCACTCACAAGACATCAACTTC
ACCCATTGTCAAGAGCTTCAACAGGGGAGAGTGTTGA
LC Amino Acid Sequence
(SEQ ID NO: 152)
EIMLMQSPGTLSLSPGERATLSCRASQSVSSSYLAWYQQKPGQAPRL
LIYGASSRATDIPDRFSGSGSGTDFTLTISRLEPEDFAVYFCQQYYG
SPWTFGQGTKVEIKRADAAPTVSIFPPSSEQLTSGGASVVCFLNNFY
PKDINVKWKIDGSERQNGVLNSWTDQDSKDSTYSMSSTLTLTKDEYE
RHNSYTCEATHKTSTSPIVKSFNRGEC
REGN7660
HCVR DNA Sequence
(SEQ ID NO: 221)
GAGGTGCAGCTGGTGGAGTCTGGGGGAGGCTTGGTCCAGCCGGGGGG
GTCCCTGAAACTCTCCTGTACAGCCTCTGGGTTGACCCTCAGTGACT
CTGCTATGCACTGGGTCCGCCAGGCTTCCGGGAAAGGGCTGGAGTGG
GTTGGCCGTATAAGAAATAAGGCTAATAGGTACGCGACAGAATATGC
TGCGTCGGTGAAAGGCAGGTTCACCATTTCAAGAGATGATTCAAAGA
ACACGGCGTATCTACAAATGAACAGCCTGAAAACCGAGGACACGGCC
GTGTATTATTGTACTAGAAACTGGAAGATTTTCCTCTTTGACTACTG
GGGCCAGGGAACCCTGGTCACCGTCTCCTCA
HCVR Amino Acid Sequence
(SEQ ID NO: 33)
EVQLVESGGGLVQPGGSLKLSCTASGLTLSDSAMHWVRQASGKGLEW
VGRIRNKANRYATEYAASVKGRFTISRDDSKNTAYLQMNSLKTEDTA
VYYCTRNWKIFLFDYWGQGTLVTVSS
HCDR1 DNA Sequence
(SEQ ID NO: 222)
GGGTTGACCCTCAGTGACTCTGCT
HCDR1 Amino Acid Sequence
(SEQ ID NO: 34)
GLTLSDSA
HCDR2 DNA Sequence
(SEQ ID NO: 223)
ATAAGAAATAAGGCTAATAGGTACGCGACA
HCDR2 Amino Acid Sequence
(SEQ ID NO: 35)
IRNKANRYAT
HCDR3 DNA Sequence
(SEQ ID NO: 224)
ACTAGAAACTGGAAGATTTTCCTCTTTGACTAC
HCDR3 Amino Acid Sequence
(SEQ ID NO: 36)
TRNWKIFLFDY
LCVR DNA Sequence
(SEQ ID NO: 225)
GAAATTGTGTTGACGCAGTCTCCAGGCACCCTGACTTTGTCTCCAGG
GGAAAGAGCCACCCTCTCCTGCAGGGCCAGTCAGAGTGTTGGCAGCA
AATACTTAGCCTGGTTCCAGCAGAAACGTGGCCAGGCTCCCAGGCTC
CTCATCTATGGTGCATCCAGCAGGACCAGTGGCATCCCCGACAGGAT
CAGTGGCAGTGGGTCAGGGACAGACTTCACTCTCACCATCAGCAGAC
TGGAGCCTGAAGATTTTGCAGTGTATTACTGTCAGCAGTATGGAAGT
TCACCCTGGACGTTCGGCCAAGGGACCAAGGTGGAAATCAAA
LCVR Amino Acid Sequence
(SEQ ID NO: 37)
EIVLTQSPGTLTLSPGERATLSCRASQSVGSKYLAWFQQKRGQAPRL
LIYGASSRTSGIPDRISGSGSGTDFTLTISRLEPEDFAVYYCQQYGS
SPWTFGQGTKVEIK
LCDR1 DNA Sequence
(SEQ ID NO: 226)
CAGAGTGTTGGCAGCAAATAC
LCDR1 Amino Acid Sequence
(SEQ ID NO: 38)
QSVGSKY
LCDR2 DNA Sequence
(SEQ ID NO: 227)
GGTGCATCC
LCDR2 Amino Acid Sequence
GAS
(SEQ ID NO: 39)
LCDR3 DNA Sequence
(SEQ ID NO: 228)
CAGCAGTATGGAAGTTCACCCTGGACG
LCDR3 Amino Acid Sequence
(SEQ ID NO: 40)
QQYGSSPWT
HC DNA Sequence
(SEQ ID NO: 229)
GAGGTGCAGCTGGTGGAGTCTGGGGGAGGCTTGGTCCAGCCGGGGGG
GTCCCTGAAACTCTCCTGTACAGCCTCTGGGTTGACCCTCAGTGACT
CTGCTATGCACTGGGTCCGCCAGGCTTCCGGGAAAGGGCTGGAGTGG
GTTGGCCGTATAAGAAATAAGGCTAATAGGTACGCGACAGAATATGC
TGCGTCGGTGAAAGGCAGGTTCACCATTTCAAGAGATGATTCAAAGA
ACACGGCGTATCTACAAATGAACAGCCTGAAAACCGAGGACACGGCC
GTGTATTATTGTACTAGAAACTGGAAGATTTTCCTCTTTGACTACTG
GGGCCAGGGAACCCTGGTCACCGTCTCCTCAGCCTCCACCAAGGGCC
CATCGGTCTTCCCCCTGGCGCCCTGCTCCAGGAGCACCTCCGAGAGC
ACAGCCGCCCTGGGCTGCCTGGTCAAGGACTACTTCCCCGAACCGGT
GACGGTGTCGTGGAACTCAGGCGCCCTGACCAGCGGCGTGCACACCT
TCCCGGCTGTCCTACAGTCCTCAGGACTCTACTCCCTCAGCAGCGTG
GTGACCGTGCCCTCCAGCAGCTTGGGCACGAAGACCTACACCTGCAA
CGTAGATCACAAGCCCAGCAACACCAAGGTGGACAAGAGAGTTGAGT
CCAAATATGGTCCCCCATGCCCACCCTGCCCAGCACCTGAGTTCCTG
GGGGGACCATCAGTCTTCCTGTTCCCCCCAAAACCCAAGGACACTCT
CATGATCTCCCGGACCCCTGAGGTCACGTGCGTGGTGGTGGACGTGA
GCCAGGAAGACCCCGAGGTCCAGTTCAACTGGTACGTGGATGGCGTG
GAGGTGCATAATGCCAAGACAAAGCCGCGGGAGGAGCAGTTCAACAG
CACGTACCGTGTGGTCAGCGTCCTCACCGTCCTGCACCAGGACTGGC
TGAACGGCAAGGAGTACAAGTGCAAGGTCTCCAACAAAGGCCTCCCG
TCCTCCATCGAGAAAACCATCTCCAAAGCCAAAGGGCAGCCCCGAGA
GCCACAGGTGTACACCCTGCCCCCATCCCAGGAGGAGATGACCAAGA
ACCAGGTCAGCCTGACCTGCCTGGTCAAAGGCTTCTACCCCAGCGAC
ATCGCCGTGGAGTGGGAGAGCAATGGGCAGCCGGAGAACAACTACAA
GACCACGCCTCCCGTGCTGGACTCCGACGGCTCCTTCTTCCTCTACA
GCAGGCTCACCGTGGACAAGAGCAGGTGGCAGGAGGGGAATGTCTTC
TCATGCTCCGTGATGCATGAGGCTCTGCACAACCACTACACACAGAA
GTCCCTCTCCCTGTCTCTGGGTAAATGA
HC Amino Acid Sequence
(SEQ ID NO: 153)
EVQLVESGGGLVQPGGSLKLSCTASGLTLSDSAMHWVRQASGKGLEW
VGRIRNKANRYATEYAASVKGRFTISRDDSKNTAYLQMNSLKTEDTA
VYYCTRNWKIFLFDYWGQGTLVTVSSASTKGPSVFPLAPCSRSTSES
TAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSD
IAVEWESNGQPENNYKTTPPVLDSDGSFFLYSRLTVDKSRWQEGNVF
SCSVMHEALHNHYTQKSLSLSLGK
LC DNA Sequence
(SEQ ID NO: 230)
GAAATTGTGTTGACGCAGTCTCCAGGCACCCTGACTTTGTCTCCAGG
GGAAAGAGCCACCCTCTCCTGCAGGGCCAGTCAGAGTGTTGGCAGCA
AATACTTAGCCTGGTTCCAGCAGAAACGTGGCCAGGCTCCCAGGCTC
CTCATCTATGGTGCATCCAGCAGGACCAGTGGCATCCCCGACAGGAT
CAGTGGCAGTGGGTCAGGGACAGACTTCACTCTCACCATCAGCAGAC
TGGAGCCTGAAGATTTTGCAGTGTATTACTGTCAGCAGTATGGAAGT
TCACCCTGGACGTTCGGCCAAGGGACCAAGGTGGAAATCAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 154)
EIVLTQSPGTLTLSPGERATLSCRASQSVGSKYLAWFQQKRGQAPRL
LIYGASSRTSGIPDRISGSGSGTDFTLTISRLEPEDFAVYYCQQYGS
SPWTFGQGTKVEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC
REGN9909
HCVR DNA Sequence
(SEQ ID NO: 231)
GAGGTGCAGCTGTTGGAGTCTGGGGGAGGCTTGGTACAGCCGGGGGG
GTCCCTGAGACTCTCCTGTGCAGCCTCTGGATTCACCTTTAACAACT
ATGGCATGAGCTGGGTCCGCCAGGGTCCAGGGAAGGGGCTGGAGTGG
GTCTCATCTATTAGTGGTAGTGGTGGTACCACATTCTACGCAGACTC
CGTGAAGGGCCGGTTCACCATCTCCAGAGACAATTCCAAGAACACGC
TGTATCTGCAAATGAACAGCCTGAGAGCCGAGGACACGGCCGTATAT
TACTGTGGCAAAGGAGGATATTGTAGTAGTAGCGGCTGCCGTCACTA
CGGTATGGACGTCTGGGGCCAAGGGACCACGGTCACCGTCTCCTCA
HCVR Amino Acid Sequence
(SEQ ID NO: 41)
EVQLLESGGGLVQPGGSLRLSCAASGFTFNNYGMSWVRQGPGKGLEW
VSSISGSGGTTFYADSVKGRFTISRDNSKNTLYLQMNSLRAEDTAVY
YCGKGGYCSSSGCRHYGMDVWGQGTTVTVSS
HCDR1 DNA Sequence
(SEQ ID NO: 232)
GGATTCACCTTTAACAACTATGGC
HCDR1 Amino Acid Sequence
(SEQ ID NO: 42)
GFTFNNYG
HCDR2 DNA Sequence
(SEQ ID NO: 233)
ATTAGTGGTAGTGGTGGTACCACA
HCDR2 Amino Acid Sequence
(SEQ ID NO: 43)
ISGSGGTT
HCDR3 DNA Sequence
(SEQ ID NO: 234)
GGCAAAGGAGGATATTGTAGTAGTAGCGGCTGCCGTCACTACGGTAT
GGACGTC
HCDR3 Amino Acid Sequence
(SEQ ID NO: 44)
GKGGYCSSSGCRHYGMDV
LCVR DNA Sequence
(SEQ ID NO: 235)
CAGTCTGTGCTGACTCAGCCACCCTCAGCGTCTGGGACCCCCGGGCA
GAGGGTCACCATCTCTTGTTCTGGAAGCAGCTCCAACATCGGAAATA
ATTATATATACTGGTACCAGCGGCTCCCAGGAACGACCCCCAAACTC
CTCATCTATAGGAATAATCAGCGGCCCTCAGGGGTCCCTGACCGATT
CTCTGGCTCCAAGTCTGGCACCTCAGCCTCCCTGGCCATCAGTGGGC
TCCGGTCCGAGGATGAGGCTGATTATTACTGTGCAGCATGGGATGAC
ACCCTGAGTGGGTATGTCTTCGGAACTGGGACCAAGGTCACCGTCCT
A
LCVR Amino Acid Sequence
(SEQ ID NO: 45)
QSVLTQPPSASGTPGQRVTISCSGSSSNIGNNYIYWYQRLPGTTPKL
LIYRNNQRPSGVPDRFSGSKSGTSASLAISGLRSEDEADYYCAAWDD
TLSGYVFGTGTKVTVL
LCDR1 DNA Sequence
(SEQ ID NO: 236)
AGCTCCAACATCGGAAATAATTAT
LCDR1 Amino Acid Sequence
(SEQ ID NO: 46)
SSNIGNNY
LCDR2 DNA Sequence
(SEQ ID NO: 237)
AGGAATAAT
LCDR2 Amino Acid Sequence
(SEQ ID NO: 47)
RNN
LCDR3 DNA Sequence
(SEQ ID NO: 238)
GCAGCATGGGATGACACCCTGAGTGGGTATGTC
LCDR3 Amino Acid Sequence
(SEQ ID NO: 48)
AAWDDTLSGYV
HC DNA Sequence
(SEQ ID NO: 239)
GAGGTGCAGCTGTTGGAGTCTGGGGGAGGCTTGGTACAGCCGGGGGG
GTCCCTGAGACTCTCCTGTGCAGCCTCTGGATTCACCTTTAACAACT
ATGGCATGAGCTGGGTCCGCCAGGGTCCAGGGAAGGGGCTGGAGTGG
GTCTCATCTATTAGTGGTAGTGGTGGTACCACATTCTACGCAGACTC
CGTGAAGGGCCGGTTCACCATCTCCAGAGACAATTCCAAGAACACGC
TGTATCTGCAAATGAACAGCCTGAGAGCCGAGGACACGGCCGTATAT
TACTGTGGCAAAGGAGGATATTGTAGTAGTAGCGGCTGCCGTCACTA
CGGTATGGACGTCTGGGGCCAAGGGACCACGGTCACCGTCTCCTCAG
CCAAAACAACAGCCCCATCGGTCTATCCACTGGCCCCTGTGTGTGGA
GATACAACTGGCTCCTCGGTGACTCTAGGATGCCTGGTCAAGGGTTA
TTTCCCTGAGCCAGTGACCTTGACCTGGAACTCTGGATCCCTGTCCA
GTGGTGTGCACACCTTCCCAGCTGTCCTGCAGTCTGACCTCTACACC
CTCAGCAGCTCAGTGACTGTAACCTCGAGCACCTGGCCCAGCCAGTC
CATCACCTGCAATGTGGCCCACCCGGCAAGCAGCACCAAGGTGGACA
AGAAAATTGAGCCCAGAGGGCCCACAATCAAGCCCTGTCCTCCATGC
AAATGCCCAGCACCTAACCTCTTGGGTGGACCATCCGTCTTCATCTT
CCCTCCAAAGATCAAGGATGTACTCATGATCTCCCTGAGCCCCATAG
TCACATGTGTGGTGGTGGATGTGAGCGAGGATGACCCAGATGTCCAG
ATCAGCTGGTTTGTGAACAACGTGGAAGTACACACAGCTCAGACACA
AACCCATAGAGAGGATTACAACAGTACTCTCCGGGTGGTCAGTGCCC
TCCCCATCCAGCACCAGGACTGGATGAGTGGCAAGGAGTTCAAATGC
AAGGTCAACAACAAAGACCTCCCAGCGCCCATCGAGAGAACCATCTC
AAAACCCAAAGGGTCAGTAAGAGCTCCACAGGTATATGTCTTGCCTC
CACCAGAAGAAGAGATGACTAAGAAACAGGTCACTCTGACCTGCATG
GTCACAGACTTCATGCCTGAAGACATTTACGTGGAGTGGACCAACAA
CGGGAAAACAGAGCTAAACTACAAGAACACTGAACCAGTCCTGGACT
CTGATGGTTCTTACTTCATGTACAGCAAGCTGAGAGTGGAAAAGAAG
AACTGGGTGGAAAGAAATAGCTACTCCTGTTCAGTGGTCCACGAGGG
TCTGCACAATCACCACACGACTAAGAGCTTCTCCCGGACTCCGGGTA
AATGA
HC Amino Acid Sequence
(SEQ ID NO: 155)
EVQLLESGGGLVQPGGSLRLSCAASGFTFNNYGMSWVRQGPGKGLEW
VSSISGSGGTTFYADSVKGRFTISRDNSKNTLYLQMNSLRAEDTAVY
YCGKGGYCSSSGCRHYGMDVWGQGTTVTVSSAKTTAPSVYPLAPVCG
DTTGSSVTLGCLVKGYFPEPVTLTWNSGSLSSGVHTFPAVLQSDLYT
LSSSVTVTSSTWPSQSITCNVAHPASSTKVDKKIEPRGPTIKPCPPC
KCPAPNLLGGPSVFIFPPKIKDVLMISLSPIVTCVVVDVSEDDPDVQ
ISWFVNNVEVHTAQTQTHREDYNSTLRVVSALPIQHQDWMSGKEFKC
KVNNKDLPAPIERTISKPKGSVRAPQVYVLPPPEEEMTKKQVTLTCM
VTDFMPEDIYVEWTNNGKTELNYKNTEPVLDSDGSYFMYSKLRVEKK
NWVERNSYSCSVVHEGLHNHHTTKSFSRTPGK
LC DNA Sequence
(SEQ ID NO: 240)
CAGTCTGTGCTGACTCAGCCACCCTCAGCGTCTGGGACCCCCGGGCA
GAGGGTCACCATCTCTTGTTCTGGAAGCAGCTCCAACATCGGAAATA
ATTATATATACTGGTACCAGCGGCTCCCAGGAACGACCCCCAAACTC
CTCATCTATAGGAATAATCAGCGGCCCTCAGGGGTCCCTGACCGATT
CTCTGGCTCCAAGTCTGGCACCTCAGCCTCCCTGGCCATCAGTGGGC
TCCGGTCCGAGGATGAGGCTGATTATTACTGTGCAGCATGGGATGAC
ACCCTGAGTGGGTATGTCTTCGGAACTGGGACCAAGGTCACCGTCCT
ACGAGCTGATGCTGCACCAACTGTATCCATCTTCCCACCATCCAGTG
AGCAGTTAACATCTGGAGGTGCCTCAGTCGTGTGCTTCTTGAACAAC
TTCTACCCCAAAGACATCAATGTCAAGTGGAAGATTGATGGCAGTGA
ACGACAAAATGGCGTCCTGAACAGTTGGACTGATCAGGACAGCAAAG
ACAGCACCTACAGCATGAGCAGCACCCTCACGTTGACCAAGGACGAG
TATGAACGACATAACAGCTATACCTGTGAGGCCACTCACAAGACATC
AACTTCACCCATTGTCAAGAGCTTCAACAGGGGAGAGTGTTGA
LC Amino Acid Sequence
(SEQ ID NO: 156)
QSVLTQPPSASGTPGQRVTISCSGSSSNIGNNYIYWYQRLPGTTPKL
LIYRNNQRPSGVPDRFSGSKSGTSASLAISGLRSEDEADYYCAAWDD
TLSGYVFGTGTKVTVLRADAAPTVSIFPPSSEQLTSGGASVVCFLNN
FYPKDINVKWKIDGSERQNGVLNSWTDQDSKDSTYSMSSTLTLTKDE
YERHNSYTCEATHKTSTSPIVKSFNRGEC
REGN10713
HCVR DNA Sequence
(SEQ ID NO: 241)
GAGGTGCAGCTGGTGGAGTCTGGGGGAAACTTGGTACAGCCTGGGGG
GTCCCTGAGACTCTCCTGTGCAGCCTCTGGATTCACCTTTACCAGCC
ATGCCATGAACTGGGTCCGCCAGGCTCCAGGGAAGGGGCTGGAGTGG
GTCTCAGTTATTACTGGTAGAGGTTTTGACACACACTACGCTGACTC
CGTGAAGGGCCGGTTCACCATCTCCAGAGACATTTCCAAAAACACGC
TGTATCTGCAAATGAACAGCCTGAGAGCCGAGGACACGGCCGTTTAT
TACTGTGCGAAAGGTCTCTATGATTCGGGGAATTATTATATCGATTA
CTGGGGCCAGGGAACCCTGGTCACCGTCTCCTCA
HCVR Amino Acid Sequence
(SEQ ID NO: 49)
EVQLVESGGNLVQPGGSLRLSCAASGFTFTSHAMNWVRQAPGKGLEW
VSVITGRGFDTHYADSVKGRFTISRDISKNTLYLQMNSLRAEDTAVY
YCAKGLYDSGNYYIDYWGQGTLVTVSS
HCDR1 DNA Sequence
(SEQ ID NO: 242)
GGATTCACCTTTACCAGCCATGCC
HCDR1 Amino Acid Sequence
(SEQ ID NO: 50)
GFTFTSHA
HCDR2 DNA Sequence
(SEQ ID NO: 243)
ATTACTGGTAGAGGTTTTGACACA
HCDR2 Amino Acid Sequence
(SEQ ID NO: 51)
ITGRGFDT
HCDR3 DNA Sequence
(SEQ ID NO: 244)
GCGAAAGGTCTCTATGATTCGGGGAATTATTATATCGATTAC
HCDR3 Amino Acid Sequence
(SEQ ID NO: 52)
AKGLYDSGNYYIDY
LCVR DNA Sequence
(SEQ ID NO: 245)
CAGTCTGTGTTGACGCAGCCGCCCTCAGTGTCTGCGGCCCCAGGACA
GAAGGTCACCATCTCCTGCTCTGGAAGCAGCTCCAACATTGGGAATA
ATTATGTTTCCTGGTACCAGCAGCTCCCAGGAACAGCCCCCAAACTC
CTCATTTATGACAATAATAAGCGACCCTCAGGGATTCCTGACCGATT
CTCTGGCTCCAAGTCTGGCACGTCAGCCACCCTGGGCATCACCGGAC
TCCAGACTGGGGACGAGGCCGATTATTACTGCGGAACATGGGATCTC
AGCCTGAGTTTCAATTGGGTGTTCGGCGGAGGGACCAAGCTGACCGT
CCTA
LCVR Amino Acid Sequence
(SEQ ID NO: 53)
QSVLTQPPSVSAAPGQKVTISCSGSSSNIGNNYVSWYQQLPGTAPKL
LIYDNNKRPSGIPDRFSGSKSGTSATLGITGLQTGDEADYYCGTWDL
SLSFNWVFGGGTKLTVL
LCDR1 DNA Sequence
(SEQ ID NO: 246)
AGCTCCAACATTGGGAATAATTAT
LCDR1 Amino Acid Sequence
(SEQ ID NO: 54)
SSNIGNNY
CDR2 DNA Sequence
(SEQ ID NO: 247)
GACAATAAT
LCDR2 Amino Acid Sequence
(SEQ ID NO: 55)
DNN
LCDR3 DNA Sequence
(SEQ ID NO: 248)
GGAACATGGGATCTCAGCCTGAGTTTCAATTGGGTG
LCDR3 Amino Acid Sequence
(SEQ ID NO: 56)
GTWDLSLSFNWV
HC DNA Sequence
(SEQ ID NO: 249)
GAGGTGCAGCTGGTGGAGTCTGGGGGAAACTTGGTACAGCCTGGGGG
GTCCCTGAGACTCTCCTGTGCAGCCTCTGGATTCACCTTTACCAGCC
ATGCCATGAACTGGGTCCGCCAGGCTCCAGGGAAGGGGCTGGAGTGG
GTCTCAGTTATTACTGGTAGAGGTTTTGACACACACTACGCTGACTC
CGTGAAGGGCCGGTTCACCATCTCCAGAGACATTTCCAAAAACACGC
TGTATCTGCAAATGAACAGCCTGAGAGCCGAGGACACGGCCGTTTAT
TACTGTGCGAAAGGTCTCTATGATTCGGGGAATTATTATATCGATTA
CTGGGGCCAGGGAACCCTGGTCACCGTCTCCTCAGCCTCCACCAAGG
GCCCATCGGTCTTCCCCCTGGCGCCCTGCTCCAGGAGCACCTCCGAG
AGCACAGCCGCCCTGGGCTGCCTGGTCAAGGACTACTTCCCCGAACC
GGTGACGGTGTCGTGGAACTCAGGCGCCCTGACCAGCGGCGTGCACA
CCTTCCCGGCTGTCCTACAGTCCTCAGGACTCTACTCCCTCAGCAGC
GTGGTGACCGTGCCCTCCAGCAGCTTGGGCACGAAGACCTACACCTG
CAACGTAGATCACAAGCCCAGCAACACCAAGGTGGACAAGAGAGTTG
AGTCCAAATATGGTCCCCCATGCCCACCCTGCCCAGCACCTGAGTTC
CTGGGGGGACCATCAGTCTTCCTGTTCCCCCCAAAACCCAAGGACAC
TCTCATGATCTCCCGGACCCCTGAGGTCACGTGCGTGGTGGTGGACG
TGAGCCAGGAAGACCCCGAGGTCCAGTTCAACTGGTACGTGGATGGC
GTGGAGGTGCATAATGCCAAGACAAAGCCGCGGGAGGAGCAGTTCAA
CAGCACGTACCGTGTGGTCAGCGTCCTCACCGTCCTGCACCAGGACT
GGCTGAACGGCAAGGAGTACAAGTGCAAGGTCTCCAACAAAGGCCTC
CCGTCCTCCATCGAGAAAACCATCTCCAAAGCCAAAGGGCAGCCCCG
AGAGCCACAGGTGTACACCCTGCCCCCATCCCAGGAGGAGATGACCA
AGAACCAGGTCAGCCTGACCTGCCTGGTCAAAGGCTTCTACCCCAGC
GACATCGCCGTGGAGTGGGAGAGCAATGGGCAGCCGGAGAACAACTA
CAAGACCACGCCTCCCGTGCTGGACTCCGACGGCTCCTTCTTCCTCT
ACAGCAGGCTCACCGTGGACAAGAGCAGGTGGCAGGAGGGGAATGTC
TTCTCATGCTCCGTGATGCATGAGGCTCTGCACAACCACTACACACA
GAAGTCCCTCTCCCTGTCTCTGGGTAAATGA
HC Amino Acid Sequence
(SEQ ID NO: 157)
EVQLVESGGNLVQPGGSLRLSCAASGFTFTSHAMNWVRQAPGKGLEW
VSVITGRGFDTHYADSVKGRFTISRDISKNTLYLQMNSLRAEDTAVY
YCAKGLYDSGNYYIDYWGQGTLVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPS
DIAVEWESNGQPENNYKTTPPVLDSDGSFFLYSRLTVDKSRWQEGNV
FSCSVMHEALHNHYTQKSLSLSLGK*
LC DNA Sequence
(SEQ ID NO: 250)
CAGTCTGTGTTGACGCAGCCGCCCTCAGTGTCTGCGGCCCCAGGACA
GAAGGTCACCATCTCCTGCTCTGGAAGCAGCTCCAACATTGGGAATA
ATTATGTTTCCTGGTACCAGCAGCTCCCAGGAACAGCCCCCAAACTC
CTCATTTATGACAATAATAAGCGACCCTCAGGGATTCCTGACCGATT
CTCTGGCTCCAAGTCTGGCACGTCAGCCACCCTGGGCATCACCGGAC
TCCAGACTGGGGACGAGGCCGATTATTACTGCGGAACATGGGATCTC
AGCCTGAGTTTCAATTGGGTGTTCGGCGGAGGGACCAAGCTGACCGT
CCTAGGCCAGCCCAAGGCCGCCCCCTCCGTGACCCTGTTCCCCCCCT
CCTCCGAGGAGCTGCAGGCCAACAAGGCCACCCTGGTGTGCCTGATC
TCCGACTTCTACCCCGGCGCCGTGACCGTGGCCTGGAAGGCCGACTC
CTCCCCCGTGAAGGCCGGCGTGGAGACCACCACCCCCTCCAAGCAGT
CCAACAACAAGTACGCCGCCTCCTCCTACCTGTCCCTGACCCCCGAG
CAGTGGAAGTCCCACCGGTCCTACTCCTGCCAGGTGACCCACGAGGG
CTCCACCGTGGAGAAGACCGTGGCCCCCACCGAGTGCTCCTGA
LC Amino Acid Sequence
(SEQ ID NO: 158)
QSVLTQPPSVSAAPGQKVTISCSGSSSNIGNNYVSWYQQLPGTAPKL
LIYDNNKRPSGIPDRFSGSKSGTSATLGITGLQTGDEADYYCGTWDL
SLSFNWVFGGGTKLTVLGQPKAAPSVTLFPPSSEELQANKATLVCLI
SDFYPGAVTVAWKADSSPVKAGVETTTPSKQSNNKYAASSYLSLTPE
QWKSHRSYSCQVTHEGSTVEKTVAPTECS*
REGN10715
HCVR DNA Sequence
(SEQ ID NO: 251)
CAGGTGCAGCTACAGCAGTGGGGCGCAGGACTGTTGAAGCCTTCGGC
GACCCTGTCCCGCACCTGCGCTGTCTATGGTGGGTCCTTCAGTGGTT
ACTACTGGAACTGGATCCGCCAGTCCCCAGGGAAGGGGCTGGAATGG
ATTGGGGAAATCCTTCATAGTGGAAGAACCAACTACAACCCGTCCCT
CAAGAGTCGAGTCACCATATCAGTAGACACGTCCAAGAACCAGTTCT
CCCTGAAGCTGACCTCTGTGACCGCCGCGGACACGGCTGTATATTAC
TGTGCGGGAAGGATAGCAGCTCGTCACGGCTGGTTCGACCCCTGGGG
CCAGGGAACCCTGGTCACCGTCTCCTCA
HCVR Amino Acid Sequence
(SEQ ID NO: 57)
QVQLQQWGAGLLKPSATLSRTCAVYGGSFSGYYWNWIRQSPGKGLEW
IGEILHSGRTNYNPSLKSRVTISVDTSKNQFSLKLTSVTAADTAVYY
CAGRIAARHGWFDPWGQGTLVTVSS
HCDR1 DNA Sequence
(SEQ ID NO: 252)
GGTGGGTCCTTCAGTGGTTACTAC
HCDR1 Amino Acid Sequence
(SEQ ID NO: 58)
GGSFSGYY
HCDR2 DNA Sequence
(SEQ ID NO: 253)
ATCCTTCATAGTGGAAGAACC
HCDR2 Amino Acid Sequence
(SEQ ID NO: 59)
ILHSGRT
HCDR3 DNA Sequence
(SEQ ID NO: 254)
GCGGGAAGGATAGCAGCTCGTCACGGCTGGTTCGACCCC
HCDR3 Amino Acid Sequence
(SEQ ID NO: 60)
AGRIAARHGWFDP
LCVR DNA Sequence
(SEQ ID NO: 255)
GACATCCAGATGACCCAGTCTCCATCTTCCGTGTCTACATCTGTAGG
AGACAGAGTCACCATCTCTTGTCGGGCGAGTCAGGATATTCGCAAGT
GGTTAGCCTGGTATCAACAGAAACCAGGAAAAGCCCCTAAACTCCTG
ATCTATGCTACATCCAGTTTGCAAAGTGGGGTCCCTTCAAGGTTCAG
CGGCAGTGGATCTGGGACAGATTTCACTCTCACCATCAGCAGCCTGC
AGCCTGAGGATTTTGCAACTTACTTTTGTCAACAGGCTAACAGTTTC
CCGTTCACTTTTGGCCAGGGGACCAAGCTGGAGATCAAA
LCVR Amino Acid Sequence
(SEQ ID NO: 61)
DIQMTQSPSSVSTSVGDRVTISCRASQDIRKWLAWYQQKPGKAPKLL
IYATSSLQSGVPSRFSGSGSGTDFTLTISSLQPEDFATYFCQQANSF
PFTFGQGTKLEIK
LCDR1 DNA Sequence
(SEQ ID NO: 256)
CAGGATATTCGCAAGTGG
LCDR1 Amino Acid Sequence
(SEQ ID NO: 62)
QDIRKW
LCDR2 DNA Sequence
(SEQ ID NO: 257)
GCTACATCC
LCDR2 Amino Acid Sequence
(SEQ ID NO: 63)
ATS
LCDR3 DNA Sequence
(SEQ ID NO: 258)
CAACAGGCTAACAGTTTCCCGTTCACT
LCDR3 Amino Acid Sequence
(SEQ ID NO: 64)
QQANSFPFT
HC DNA Sequence
(SEQ ID NO: 259)
CAGGTGCAGCTACAGCAGTGGGGCGCAGGACTGTTGAAGCCTTCGGC
GACCCTGTCCCGCACCTGCGCTGTCTATGGTGGGTCCTTCAGTGGTT
ACTACTGGAACTGGATCCGCCAGTCCCCAGGGAAGGGGCTGGAATGG
ATTGGGGAAATCCTTCATAGTGGAAGAACCAACTACAACCCGTCCCT
CAAGAGTCGAGTCACCATATCAGTAGACACGTCCAAGAACCAGTTCT
CCCTGAAGCTGACCTCTGTGACCGCCGCGGACACGGCTGTATATTAC
TGTGCGGGAAGGATAGCAGCTCGTCACGGCTGGTTCGACCCCTGGGG
CCAGGGAACCCTGGTCACCGTCTCCTCAGCCTCCACCAAGGGCCCAT
CGGTCTTCCCCCTGGCGCCCTGCTCCAGGAGCACCTCCGAGAGCACA
GCCGCCCTGGGCTGCCTGGTCAAGGACTACTTCCCCGAACCGGTGAC
GGTGTCGTGGAACTCAGGCGCCCTGACCAGCGGCGTGCACACCTTCC
CGGCTGTCCTACAGTCCTCAGGACTCTACTCCCTCAGCAGCGTGGTG
ACCGTGCCCTCCAGCAGCTTGGGCACGAAGACCTACACCTGCAACGT
AGATCACAAGCCCAGCAACACCAAGGTGGACAAGAGAGTTGAGTCCA
AATATGGTCCCCCATGCCCACCCTGCCCAGCACCTGAGTTCCTGGGG
GGACCATCAGTCTTCCTGTTCCCCCCAAAACCCAAGGACACTCTCAT
GATCTCCCGGACCCCTGAGGTCACGTGCGTGGTGGTGGACGTGAGCC
AGGAAGACCCCGAGGTCCAGTTCAACTGGTACGTGGATGGCGTGGAG
GTGCATAATGCCAAGACAAAGCCGCGGGAGGAGCAGTTCAACAGCAC
GTACCGTGTGGTCAGCGTCCTCACCGTCCTGCACCAGGACTGGCTGA
ACGGCAAGGAGTACAAGTGCAAGGTCTCCAACAAAGGCCTCCCGTCC
TCCATCGAGAAAACCATCTCCAAAGCCAAAGGGCAGCCCCGAGAGCC
ACAGGTGTACACCCTGCCCCCATCCCAGGAGGAGATGACCAAGAACC
AGGTCAGCCTGACCTGCCTGGTCAAAGGCTTCTACCCCAGCGACATC
GCCGTGGAGTGGGAGAGCAATGGGCAGCCGGAGAACAACTACAAGAC
CACGCCTCCCGTGCTGGACTCCGACGGCTCCTTCTTCCTCTACAGCA
GGCTCACCGTGGACAAGAGCAGGTGGCAGGAGGGGAATGTCTTCTCA
TGCTCCGTGATGCATGAGGCTCTGCACAACCACTACACACAGAAGTC
CCTCTCCCTGTCTCTGGGTAAATGA
HC Amino Acid Sequence
(SEQ ID NO: 159)
QVQLQQWGAGLLKPSATLSRTCAVYGGSFSGYYWNWIRQSPGKGLEW
IGEILHSGRTNYNPSLKSRVTISVDTSKNQFSLKLTSVTAADTAVYY
CAGRIAARHGWFDPWGQGTLVTVSSASTKGPSVFPLAPCSRSTSEST
AALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVE
VHNAKTKPREEQFNSTYRWVSVLTVLHQDWLNGKEYKCKVSNKGLPS
SIEKTISKAKGQPREPQVYTLPPSQEEMTKNQVSLTCLVKGFYPSDI
AVEWESNGQPENNYKTTPPVLDSDGSFFLYSRLTVDKSRWQEGNVFS
CSVMHEALHNHYTQKSLSLSLGK*
LC DNA Sequence
(SEQ ID NO: 260)
GACATCCAGATGACCCAGTCTCCATCTTCCGTGTCTACATCTGTAGG
AGACAGAGTCACCATCTCTTGTCGGGCGAGTCAGGATATTCGCAAGT
GGTTAGCCTGGTATCAACAGAAACCAGGAAAAGCCCCTAAACTCCTG
ATCTATGCTACATCCAGTTTGCAAAGTGGGGTCCCTTCAAGGTTCAG
CGGCAGTGGATCTGGGACAGATTTCACTCTCACCATCAGCAGCCTGC
AGCCTGAGGATTTTGCAACTTACTTTTGTCAACAGGCTAACAGTTTC
CCGTTCACTTTTGGCCAGGGGACCAAGCTGGAGATCAAACGAACTGT
GGCTGCACCATCTGTCTTCATCTTCCCGCCATCTGATGAGCAGTTGA
AATCTGGAACTGCCTCTGTTGTGTGCCTGCTGAATAACTTCTATCCC
AGAGAGGCCAAAGTACAGTGGAAGGTGGATAACGCCCTCCAATCGGG
TAACTCCCAGGAGAGTGTCACAGAGCAGGACAGCAAGGACAGCACCT
ACAGCCTCAGCAGCACCCTGACGCTGAGCAAAGCAGACTACGAGAAA
CACAAAGTCTACGCCTGCGAAGTCACCCATCAGGGCCTGAGCTCGCC
CGTCACAAAGAGCTTCAACAGGGGAGAGTGTTAG
LC Amino Acid Sequence
(SEQ ID NO: 160)
DIQMTQSPSSVSTSVGDRVTISCRASQDIRKWLAWYQQKPGKAPKLL
IYATSSLQSGVPSRFSGSGSGTDFTLTISSLQPEDFATYFCQQANSF
PFTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC*
REGN10716
HCVR DNA Sequence
(SEQ ID NO: 261)
CAGGTGCAGCTGCAGGAGTCGGGCCCAGGACTGGTGAAGCCTTCGGA
GACCCTGTCCCTCACCTGCACTGTCTCTGGTGACTCCATCAATAATT
ACTACTGGACCTGGCTCCGGCAGCCCCCAGGGAAGGGACTGGAGTGG
ATTGGTTATATCTATTACAGTGGGAGCGCCAACTACAACCCCTCCCT
CAAGAGTCGAGTCACCATATCAGTAGACACGTCCAAGAACCAGTTCT
CCCTGAAGCTAAATTCTGTGACCGCTGCGGACACGGCCGTGTATTAC
TGTGCGAGAGGGGCGGTCAAGTACTTCCGGCATTGGGGCCAGGGCAC
CCTGGTCACCGTCTCCTCA
HCVR Amino Acid Sequence
(SEQ ID NO: 65)
QVQLQESGPGLVKPSETLSLTCTVSGDSINNYYWTWLRQPPGKGLEW
IGYIYYSGSANYNPSLKSRVTISVDTSKNQFSLKLNSVTAADTAVYY
CARGAVKYFRHWGQGTLVTVSS
HCDR1 DNA Sequence
(SEQ ID NO: 262)
GGTGACTCCATCAATAATTACTAC
HCDR1 Amino Acid Sequence
(SEQ ID NO: 66)
GDSINNYY
HCDR2 DNA Sequence
(SEQ ID NO: 263)
ATCTATTACAGTGGGAGCGCC
HCDR2 Amino Acid Sequence
(SEQ ID NO: 67)
IYYSGSA
HCDR3 DNA Sequence
(SEQ ID NO: 264)
GCGAGAGGGGCGGTCAAGTACTTCCGGCAT
HCDR3 Amino Acid Sequence
(SEQ ID NO: 68)
ARGAVKYFRH
LCVR DNA Sequence
(SEQ ID NO: 265)
GAAATTGTGTTGACGCAGTCTCCGGGCACCCTCTCTTTGTCTCCAGG
GGAAAGAGCCACCCTCTCCTGCAGGGCCAGTCAGACTATTAACCACA
ACAACTTAGCCTGGTACCAGCAGAGACCTGGCCAGGCTCCCAGGCTC
CTCATCTATGGTGCATCCAACAGGGCCACTGCCATCCCAGACAGGTT
CAGTGGCAGTGGGTCTGGGACAGACTTCACTCTCACCATCAGCAGAC
TGGAGCCTGAAGATTTTGAAGTGTATTCTTGTCAGCAGTATGGTAGC
TTGCCGCTCACTTTCGGCGGAGGGACCAAGGTGGAGATCAAA
LCVR Amino Acid Sequence
(SEQ ID NO: 69)
EIVLTQSPGTLSLSPGERATLSCRASQTINHNNLAWYQQRPGQAPRL
LIYGASNRATAIPDRFSGSGSGTDFTLTISRLEPEDFEVYSCQQYGS
LPLTFGGGTKVEIK
LCDR1 DNA Sequence
(SEQ ID NO: 266)
CAGACTATTAACCACAACAAC
LCDR1 Amino Acid Sequence
(SEQ ID NO: 70)
QTINHNN
LCDR2 DNA Sequence
(SEQ ID NO: 267)
GGTGCATCC
LCDR2 Amino Acid Sequence
(SEQ ID NO: 71)
GAS
LCDR3 DNA Sequence
(SEQ ID NO: 268)
CAGCAGTATGGTAGCTTGCCGCTCACT
LCDR3 Amino Acid Sequence
(SEQ ID NO: 72)
QQYGSLPLT
HC DNA Sequence
(SEQ ID NO: 269)
CAGGTGCAGCTGCAGGAGTCGGGCCCAGGACTGGTGAAGCCTTCGGA
GACCCTGTCCCTCACCTGCACTGTCTCTGGTGACTCCATCAATAATT
ACTACTGGACCTGGCTCCGGCAGCCCCCAGGGAAGGGACTGGAGTGG
ATTGGTTATATCTATTACAGTGGGAGCGCCAACTACAACCCCTCCCT
CAAGAGTCGAGTCACCATATCAGTAGACACGTCCAAGAACCAGTTCT
CCCTGAAGCTAAATTCTGTGACCGCTGCGGACACGGCCGTGTATTAC
TGTGCGAGAGGGGCGGTCAAGTACTTCCGGCATTGGGGCCAGGGCAC
CCTGGTCACCGTCTCCTCAGCCTCCACCAAGGGCCCATCGGTCTTCC
CCCTGGCGCCCTGCTCCAGGAGCACCTCCGAGAGCACAGCCGCCCTG
GGCTGCCTGGTCAAGGACTACTTCCCCGAACCGGTGACGGTGTCGTG
GAACTCAGGCGCCCTGACCAGCGGCGTGCACACCTTCCCGGCTGTCC
TACAGTCCTCAGGACTCTACTCCCTCAGCAGCGTGGTGACCGTGCCC
TCCAGCAGCTTGGGCACGAAGACCTACACCTGCAACGTAGATCACAA
GCCCAGCAACACCAAGGTGGACAAGAGAGTTGAGTCCAAATATGGTC
CCCCATGCCCACCCTGCCCAGCACCTGAGTTCCTGGGGGGACCATCA
GTCTTCCTGTTCCCCCCAAAACCCAAGGACACTCTCATGATCTCCCG
GACCCCTGAGGTCACGTGCGTGGTGGTGGACGTGAGCCAGGAAGACC
CCGAGGTCCAGTTCAACTGGTACGTGGATGGCGTGGAGGTGCATAAT
GCCAAGACAAAGCCGCGGGAGGAGCAGTTCAACAGCACGTACCGTGT
GGTCAGCGTCCTCACCGTCCTGCACCAGGACTGGCTGAACGGCAAGG
AGTACAAGTGCAAGGTCTCCAACAAAGGCCTCCCGTCCTCCATCGAG
AAAACCATCTCCAAAGCCAAAGGGCAGCCCCGAGAGCCACAGGTGTA
CACCCTGCCCCCATCCCAGGAGGAGATGACCAAGAACCAGGTCAGCC
TGACCTGCCTGGTCAAAGGCTTCTACCCCAGCGACATCGCCGTGGAG
TGGGAGAGCAATGGGCAGCCGGAGAACAACTACAAGACCACGCCTCC
CGTGCTGGACTCCGACGGCTCCTTCTTCCTCTACAGCAGGCTCACCG
TGGACAAGAGCAGGTGGCAGGAGGGGAATGTCTTCTCATGCTCCGTG
ATGCATGAGGCTCTGCACAACCACTACACACAGAAGTCCCTCTCCCT
GTCTCTGGGTAAATGA
HC Amino Acid Sequence
(SEQ ID NO: 161)
QVQLQESGPGLVKPSETLSLTCTVSGDSINNYYWTWLRQPPGKGLEW
IGYIYYSGSANYNPSLKSRVTISVDTSKNQFSLKLNSVTAADTAVYY
CARGAVKYFRHWGQGTLVTVSSASTKGPSVFPLAPCSRSTSESTAAL
GCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVE
WESNGQPENNYKTTPPVLDSDGSFFLYSRLTVDKSRWQEGNVFSCSV
MHEALHNHYTQKSLSLSLGK*
LC DNA Sequence
(SEQ ID NO: 270)
GAAATTGTGTTGACGCAGTCTCCGGGCACCCTCTCTTTGTCTCCAGG
GGAAAGAGCCACCCTCTCCTGCAGGGCCAGTCAGACTATTAACCACA
ACAACTTAGCCTGGTACCAGCAGAGACCTGGCCAGGCTCCCAGGCTC
CTCATCTATGGTGCATCCAACAGGGCCACTGCCATCCCAGACAGGTT
CAGTGGCAGTGGGTCTGGGACAGACTTCACTCTCACCATCAGCAGAC
TGGAGCCTGAAGATTTTGAAGTGTATTCTTGTCAGCAGTATGGTAGC
TTGCCGCTCACTTTCGGCGGAGGGACCAAGGTGGAGATCAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 162)
EIVLTQSPGTLSLSPGERATLSCRASQTINHNNL
AWYQQRPGQAPRLLIYGASNRATAIPDRFSGSGSGTDFTLTISRLEP
EDFEVYSCQQYGSLPLTFGGGTKVEIKRTVAAPSVFIFPPSDEQLKS
GTASVVCLLNNFYPREAKVQWKVDNALQSGNSQESVTEQDSKDSTYS
LSSTLTLSKADYEKHKVYACEVTHQGLSSPVTKSFNRGEC*
REGN10717
HCVR DNA Sequence
(SEQ ID NO: 271)
CAGGTGCAGCTGGTGGAGTCTGGGGGAGGCGTGGTCCAGCCTGGGAG
GTCCCTGAGACTCTCCTGTGCAGCGTCTGGATTCACCTTCAGTACAT
ATGGCATGCACTGGGTCCGCCAGGCTCCAGGCAAGGGGCTGGAGTGG
GTGGCAGTTATTTGGCATGATGGAAGTGATAAATATTATGTAGACTC
CGTGAAGGGCCGATTCTCCATCGCCAGAGACAATTCCAAGAACACGC
TTTATCTGCAAATGAATAGTCTGAGAGTCGAGGACACGGGTATATAT
TACTGTGCGAGAAGGGGTATACGTGGAACCGTTTTTGACCACTGGGG
CCTGGGAACCCTGGTCACCGTCTCCTCA
HCVR Amino Acid Sequence
(SEQ ID NO: 73)
QVQLVESGGGVVQPGRSLRLSCAASGFTFSTYGMHWVRQAPGKGLEW
VAVIWHDGSDKYYVDSVKGRFSIARDNSKNTLYLQMNSLRVEDTGIY
YCARRGIRGTVFDHWGLGTLVTVSS
HCDR1 DNA Sequence
(SEQ ID NO: 272)
GGATTCACCTTCAGTACATATGGC
HCDR1 Amino Acid Sequence
(SEQ ID NO: 74)
GFTFSTYG
HCDR2 DNA Sequence
(SEQ ID NO: 273)
ATTTGGCATGATGGAAGTGATAAA
HCDR2 Amino Acid Sequence
(SEQ ID NO: 75)
IWHDGSDK
HCDR3 DNA Sequence
(SEQ ID NO: 274)
GCGAGAAGGGGTATACGTGGAACCGTTTTTGACCAC
HCDR3 Amino Acid Sequence
(SEQ ID NO: 76)
ARRGIRGTVFDH
LCVR DNA Sequence
(SEQ ID NO: 275)
GACATCCAGATGACCCAGTCTCCTTCCACCCTGTCTGCATCTGTAGG
AGACAGAGTCACCCTCACTTGTCGGGCCAGTCAGAGTATTAGTAACA
AGTTGGCCTGGTATCAGCAGAAACCAGGGAAAGCCCCTAACCTCCTG
ATCTATAAGGCGTCTAATTTAGAAAGTGGGGTCCCATCAAGGTTCAG
CGGCAGTGGATCTGGGACAGAATTCACTCTCACCATCAGCAGCCTGC
AGCCTGATGATTTTGCAACTTATTACTGCCAACAGTATAATAGTTAT
TCGTGGACGTTCGGCCAAGGGACCAAGGTGGAAATCAAA
LCVR Amino Acid Sequence
(SEQ ID NO: 77)
DIQMTQSPSTLSASVGDRVTLTCRASQSISNKLAWYQQKPGKAPNLL
IYKASNLESGVPSRFSGSGSGTEFTLTISSLQPDDFATYYCQQYNSY
SWTFGQGTKVEIK
LCDR1 DNA Sequence
(SEQ ID NO: 276)
CAGAGTATTAGTAACAAG
LCDR1 Amino Acid Sequence
(SEQ ID NO: 78)
QSISNK
LCDR2 DNA Sequence
(SEQ ID NO: 277)
AAGGCGTCT
LCDR2 Amino Acid Sequence
(SEQ ID NO: 79)
KAS
LCDR3 DNA Sequence
(SEQ ID NO: 278)
CAACAGTATAATAGTTATTCGTGGACG
LCDR3 Amino Acid Sequence
(SEQ ID NO: 80)
QQYNSYSWT
HC DNA Sequence
(SEQ ID NO: 279)
CAGGTGCAGCTGGTGGAGTCTGGGGGAGGCGTGGTCCAGCCTGGGAG
GTCCCTGAGACTCTCCTGTGCAGCGTCTGGATTCACCTTCAGTACAT
ATGGCATGCACTGGGTCCGCCAGGCTCCAGGCAAGGGGCTGGAGTGG
GTGGCAGTTATTTGGCATGATGGAAGTGATAAATATTATGTAGACTC
CGTGAAGGGCCGATTCTCCATCGCCAGAGACAATTCCAAGAACACGC
TTTATCTGCAAATGAATAGTCTGAGAGTCGAGGACACGGGTATATAT
TACTGTGCGAGAAGGGGTATACGTGGAACCGTTTTTGACCACTGGGG
CCTGGGAACCCTGGTCACCGTCTCCTCAGCCTCCACCAAGGGCCCAT
CGGTCTTCCCCCTGGCGCCCTGCTCCAGGAGCACCTCCGAGAGCACA
GCCGCCCTGGGCTGCCTGGTCAAGGACTACTTCCCCGAACCGGTGAC
GGTGTCGTGGAACTCAGGCGCCCTGACCAGCGGCGTGCACACCTTCC
CGGCTGTCCTACAGTCCTCAGGACTCTACTCCCTCAGCAGCGTGGTG
ACCGTGCCCTCCAGCAGCTTGGGCACGAAGACCTACACCTGCAACGT
AGATCACAAGCCCAGCAACACCAAGGTGGACAAGAGAGTTGAGTCCA
AATATGGTCCCCCATGCCCACCCTGCCCAGCACCTGAGTTCCTGGGG
GGACCATCAGTCTTCCTGTTCCCCCCAAAACCCAAGGACACTCTCAT
GATCTCCCGGACCCCTGAGGTCACGTGCGTGGTGGTGGACGTGAGCC
AGGAAGACCCCGAGGTCCAGTTCAACTGGTACGTGGATGGCGTGGAG
GTGCATAATGCCAAGACAAAGCCGCGGGAGGAGCAGTTCAACAGCAC
GTACCGTGTGGTCAGCGTCCTCACCGTCCTGCACCAGGACTGGCTGA
ACGGCAAGGAGTACAAGTGCAAGGTCTCCAACAAAGGCCTCCCGTCC
TCCATCGAGAAAACCATCTCCAAAGCCAAAGGGCAGCCCCGAGAGCC
ACAGGTGTACACCCTGCCCCCATCCCAGGAGGAGATGACCAAGAACC
AGGTCAGCCTGACCTGCCTGGTCAAAGGCTTCTACCCCAGCGACATC
GCCGTGGAGTGGGAGAGCAATGGGCAGCCGGAGAACAACTACAAGAC
CACGCCTCCCGTGCTGGACTCCGACGGCTCCTTCTTCCTCTACAGCA
GGCTCACCGTGGACAAGAGCAGGTGGCAGGAGGGGAATGTCTTCTCA
TGCTCCGTGATGCATGAGGCTCTGCACAACCACTACACACAGAAGTC
CCTCTCCCTGTCTCTGGGTAAATGA
HC Amino Acid Sequence
(SEQ ID NO: 163)
QVQLVESGGGVVQPGRSLRLSCAASGFTFSTYGMHWVRQAPGKGLEW
VAVIWHDGSDKYYVDSVKGRFSIARDNSKNTLYLQMNSLRVEDTGIY
YCARRGIRGTVFDHWGLGTLVTVSSASTKGPSVFPLAPCSRSTSEST
AALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVE
VHNAKTKPREEQFNSTYRWVSVLTVLHQDWLNGKEYKCKVSNKGLPS
SIEKTISKAKGQPREPQVYTLPPSQEEMTKNQVSLTCLVKGFYPSDI
AVEWESNGQPENNYKTTPPVLDSDGSFFLYSRLTVDKSRWQEGNVFS
CSVMHEALHNHYTQKSLSLSLGK*
LC DNA Sequence
(SEQ ID NO: 280)
GACATCCAGATGACCCAGTCTCCTTCCACCCTGTCTGCATCTGTAGG
AGACAGAGTCACCCTCACTTGTCGGGCCAGTCAGAGTATTAGTAACA
AGTTGGCCTGGTATCAGCAGAAACCAGGGAAAGCCCCTAACCTCCTG
ATCTATAAGGCGTCTAATTTAGAAAGTGGGGTCCCATCAAGGTTCAG
CGGCAGTGGATCTGGGACAGAATTCACTCTCACCATCAGCAGCCTGC
AGCCTGATGATTTTGCAACTTATTACTGCCAACAGTATAATAGTTAT
TCGTGGACGTTCGGCCAAGGGACCAAGGTGGAAATCAAACGAACTGT
GGCTGCACCATCTGTCTTCATCTTCCCGCCATCTGATGAGCAGTTGA
AATCTGGAACTGCCTCTGTTGTGTGCCTGCTGAATAACTTCTATCCC
AGAGAGGCCAAAGTACAGTGGAAGGTGGATAACGCCCTCCAATCGGG
TAACTCCCAGGAGAGTGTCACAGAGCAGGACAGCAAGGACAGCACCT
ACAGCCTCAGCAGCACCCTGACGCTGAGCAAAGCAGACTACGAGAAA
CACAAAGTCTACGCCTGCGAAGTCACCCATCAGGGCCTGAGCTCGCC
CGTCACAAAGAGCTTCAACAGGGGAGAGTGTTAG
LC Amino Acid Sequence
(SEQ ID NO: 164)
DIQMTQSPSTLSASVGDRVTLTCRASQSISNKLAWYQQKPGKAPNLL
IYKASNLESGVPSRFSGSGSGTEFTLTISSLQPDDFATYYCQQYNSY
SWTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC*
REGN10783
HCVR DNA Sequence
(SEQ ID NO: 281)
CAGGTGCAGCTGGTGGAGTCTGGGGGAGGCGTGGTCCAGCCTGGGACG
TCCCTGAGACTCTCCTGTGCAGCGTCAGGATTCACCTTCAGTAGCTA
TGGCATGCACTGGGTCCGCCAGGCTCCAGGCAAGGGGCTGGAGTGGG
TGGCAGTTATATGGATTGATGGAAGTAATAAATATTATGCAGACTCC
GTGAAGGGCCGATTCACCATCTCCAGAGACAATTCCAAGAACACGCT
GTATCTGCAAATGAACAGCCTGAGAGCCGAGGACACGGCTGTGTATT
ACTGTGCGAGAAGGGGGGGTATAGTAGTAGCTGCCCCCTTTGACTAC
TGGGGCCAGGGAACCCTGGTCACCGTCTCCTCA
HCVR Amino Acid Sequence
(SEQ ID NO: 81)
QVQLVESGGGVVQPGTSLRLSCAASGFTFSSYGMHWVRQAPGKGLEW
VAVIWIDGSNKYYADSVKGRFTISRDNSKNTLYLQMNSLRAEDTAVY
YCARRGGIVVAAPFDYWGQGTLVTVSS
HCDR1 DNA Sequence
(SEQ ID NO: 282)
GGATTCACCTTCAGTAGCTATGGC
HCDR1 Amino Acid Sequence
(SEQ ID NO: 82)
GFTFSSYG
HCDR2 DNA Sequence
(SEQ ID NO: 283)
ATATGGATTGATGGAAGTAATAAA
HCDR2 Amino Acid Sequence
(SEQ ID NO: 83)
IWIDGSNK
HCDR3 DNA Sequence
(SEQ ID NO: 284)
GCGAGAAGGGGGGGTATAGTAGTAGCTGCCCCCTTTGACTAC
HCDR3 Amino Acid Sequence
(SEQ ID NO: 84)
ARRGGIVVAAPFDY
LCVR DNA Sequence
(SEQ ID NO: 285)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAA
LCVR Amino Acid Sequence
(SEQ ID NO: 85)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIK
LCDR1 DNA Sequence
(SEQ ID NO: 286)
CAGAGCATTAGCAGCTAT
LCDR1 Amino Acid Sequence
(SEQ ID NO: 86)
QSISSY
LCDR2 DNA Sequence
(SEQ ID NO: 287)
GCTGCATCC
LCDR2 Amino Acid Sequence
(SEQ ID NO: 87)
AAS
LCDR3 DNA Sequence
(SEQ ID NO: 288)
CAACAGAGTTACAGTACCCCTCCGATCACC
LCDR3 Amino Acid Sequence
(SEQ ID NO: 88)
QQSYSTPPIT
HC DNA Sequence
(SEQ ID NO: 289)
CAGGTGCAGCTGGTGGAGTCTGGGGGAGGCGTGGTCCAGCCTGGGAC
GTCCCTGAGACTCTCCTGTGCAGCGTCAGGATTCACCTTCAGTAGCT
ATGGCATGCACTGGGTCCGCCAGGCTCCAGGCAAGGGGCTGGAGTGG
GTGGCAGTTATATGGATTGATGGAAGTAATAAATATTATGCAGACTC
CGTGAAGGGCCGATTCACCATCTCCAGAGACAATTCCAAGAACACGC
TGTATCTGCAAATGAACAGCCTGAGAGCCGAGGACACGGCTGTGTAT
TACTGTGCGAGAAGGGGGGGTATAGTAGTAGCTGCCCCCTTTGACTA
CTGGGGCCAGGGAACCCTGGTCACCGTCTCCTCAGCCTCCACCAAGG
GCCCATCGGTCTTCCCCCTGGCGCCCTGCTCCAGGAGCACCTCCGAG
AGCACAGCCGCCCTGGGCTGCCTGGTCAAGGACTACTTCCCCGAACC
GGTGACGGTGTCGTGGAACTCAGGCGCCCTGACCAGCGGCGTGCACA
CCTTCCCGGCTGTCCTACAGTCCTCAGGACTCTACTCCCTCAGCAGC
GTGGTGACCGTGCCCTCCAGCAGCTTGGGCACGAAGACCTACACCTG
CAACGTAGATCACAAGCCCAGCAACACCAAGGTGGACAAGAGAGTTG
AGTCCAAATATGGTCCCCCATGCCCACCCTGCCCAGCACCTGAGTTC
CTGGGGGGACCATCAGTCTTCCTGTTCCCCCCAAAACCCAAGGACAC
TCTCATGATCTCCCGGACCCCTGAGGTCACGTGCGTGGTGGTGGACG
TGAGCCAGGAAGACCCCGAGGTCCAGTTCAACTGGTACGTGGATGGC
GTGGAGGTGCATAATGCCAAGACAAAGCCGCGGGAGGAGCAGTTCAA
CAGCACGTACCGTGTGGTCAGCGTCCTCACCGTCCTGCACCAGGACT
GGCTGAACGGCAAGGAGTACAAGTGCAAGGTCTCCAACAAAGGCCTC
CCGTCCTCCATCGAGAAAACCATCTCCAAAGCCAAAGGGCAGCCCCG
AGAGCCACAGGTGTACACCCTGCCCCCATCCCAGGAGGAGATGACCA
AGAACCAGGTCAGCCTGACCTGCCTGGTCAAAGGCTTCTACCCCAGC
GACATCGCCGTGGAGTGGGAGAGCAATGGGCAGCCGGAGAACAACTA
CAAGACCACGCCTCCCGTGCTGGACTCCGACGGCTCCTTCTTCCTCT
ACAGCAGGCTCACCGTGGACAAGAGCAGGTGGCAGGAGGGGAATGTC
TTCTCATGCTCCGTGATGCATGAGGCTCTGCACAACCACTACACACA
GAAGTCCCTCTCCCTGTCTCTGGGTAAATGA
HC Amino Acid Sequence
(SEQ ID NO: 165)
QVQLVESGGGVVQPGTSLRLSCAASGFTFSSYGMHWVRQAPGKGLEW
VAVIWIDGSNKYYADSVKGRFTISRDNSKNTLYLQMNSLRAEDTAVY
YCARRGGIVVAAPFDYWGQGTLVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPS
DIAVEWESNGQPENNYKTTPPVLDSDGSFFLYSRLTVDKSRWQEGNV
FSCSVMHEALHNHYTQKSLSLSLGK*
LC DNA Sequence
(SEQ ID NO: 290)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 166)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC*
REGN7854
HCVR DNA Sequence
(SEQ ID NO: 291)
CAGGTTCAGCTGGTGCAGTCTGGAGCTGAGGTGAAGAAGCCTGGGGC
CTCAGTGAAGGTCTCCTGCAAGGCTTCTGGTTACGCCTTCACCACCT
ATGGTATCACCTGGGTGCGACAGGCCCCTGGACAAGGACTTGAGTGG
ATGGGATGGATCAGCGCTTACAATGGAAATACAAACTATGCAGAGAA
GGTCCAGGGCAGATTCACCATGACCACAGACACATCCACGAATACAG
CCTACATGGAGCTGAGGAGCCTGAGATCCGACGACACGGCCGTGTAT
TTCTGTGCGAGAAAGGGTCACTATGGTTCGGGGACTTATTATAACCC
CTTTGGTTTTGATTTTTGGGGCCAAGGGACAATGGTCACCGTCTCTT
CA
HCVR Amino Acid Sequence
(SEQ ID NO: 89)
QVQLVQSGAEVKKPGASVKVSCKASGYAFTTYGITWVRQAPGQGLEW
MGWISAYNGNTNYAEKVQGRFTMTTDTSTNTAYMELRSLRSDDTAVY
FCARKGHYGSGTYYNPFGFDFWGQGTMVTVSS
HCDR1 DNA Sequence
(SEQ ID NO: 292)
GGTTACGCCTTCACCACCTATGGT
HCDR1 Amino Acid Sequence
(SEQ ID NO: 90)
GYAFTTYG
HCDR2 DNA Sequence
(SEQ ID NO: 293)
ATCAGCGCTTACAATGGAAATACA
HCDR2 Amino Acid Sequence
(SEQ ID NO: 91)
ISAYNGNT
HCDR3 DNA Sequence
(SEQ ID NO: 294)
GCGAGAAAGGGTCACTATGGTTCGGGGACTTATTATAACCCCTTTGG
TTTTGATTTT
HCDR3 Amino Acid Sequence
(SEQ ID NO: 92)
ARKGHYGSGTYYNPFGFDF
LCVR DNA Sequence
(SEQ ID NO: 295)
GAAATTGTGTTGACGCAGTCTCCAGGCACCCTGTCTTTGTCTCCAGG
GGAAAGAGCCACCCTCTCCTGCAGGGCCAGTCAGAGTGTTAGCAGCA
GCTACTTAGCCTGGTACCAACAGAAACCTGGCCAGGCTCCCAGGCTC
CTCATCTATGGTGCATCCAGCAGGGCCACTGGCATCCCAGACAGGTT
CAGTGGCAGTGGGTCTGGGACAGACTTCACTCTCACCATCAGCAGAC
TGGAGCCTGAAGATTTTGCTTTGTATTTCTGTCAGCAGTATTATGGC
TCACCTTGGACGTTCGGCCAAGGGACCAAGGTGGAAATCAAA
LCVR Amino Acid Sequence
(SEQ ID NO: 93)
EIVLTQSPGTLSLSPGERATLSCRASQSVSSSYLAWYQQKPGQAPRL
LIYGASSRATGIPDRFSGSGSGTDFTLTISRLEPEDFALYFCQQYYG
SPWTFGQGTKVEIK
LCDR1 DNA Sequence
(SEQ ID NO: 296)
CAGAGTGTTAGCAGCAGCTAC
LCDR1 Amino Acid Sequence
(SEQ ID NO: 94)
QSVSSSY
LCDR2 DNA Sequence
(SEQ ID NO: 297)
GGTGCATCC
LCDR2 Amino Acid Sequence
(SEQ ID NO: 95)
GAS
LCDR3 DNA Sequence
(SEQ ID NO: 298)
CAGCAGTATTATGGCTCACCTTGGACG
LCDR3 Amino Acid Sequence
(SEQ ID NO: 96)
QQYYGSPWT
HC DNA Sequence
(SEQ ID NO: 299)
CAGGTTCAGCTGGTGCAGTCTGGAGCTGAGGTGAAGAAGCCTGGGGC
CTCAGTGAAGGTCTCCTGCAAGGCTTCTGGTTACGCCTTCACCACCT
ATGGTATCACCTGGGTGCGACAGGCCCCTGGACAAGGACTTGAGTGG
ATGGGATGGATCAGCGCTTACAATGGAAATACAAACTATGCAGAGAA
GGTCCAGGGCAGATTCACCATGACCACAGACACATCCACGAATACAG
CCTACATGGAGCTGAGGAGCCTGAGATCCGACGACACGGCCGTGTAT
TTCTGTGCGAGAAAGGGTCACTATGGTTCGGGGACTTATTATAACCC
CTTTGGTTTTGATTTTTGGGGCCAAGGGACAATGGTCACCGTCTCTT
CAGCCTCCACCAAGGGCCCATCGGTCTTCCCCCTGGCGCCCTGCTCC
AGGAGCACCTCCGAGAGCACAGCCGCCCTGGGCTGCCTGGTCAAGGA
CTACTTCCCCGAACCGGTGACGGTGTCGTGGAACTCAGGCGCCCTGA
CCAGCGGCGTGCACACCTTCCCGGCTGTCCTACAGTCCTCAGGACTC
TACTCCCTCAGCAGCGTGGTGACCGTGCCCTCCAGCAGCTTGGGCAC
GAAGACCTACACCTGCAACGTAGATCACAAGCCCAGCAACACCAAGG
TGGACAAGAGAGTTGAGTCCAAATATGGTCCCCCATGCCCACCCTGC
CCAGCACCTGAGTTCCTGGGGGGACCATCAGTCTTCCTGTTCCCCCC
AAAACCCAAGGACACTCTCATGATCTCCCGGACCCCTGAGGTCACGT
GCGTGGTGGTGGACGTGAGCCAGGAAGACCCCGAGGTCCAGTTCAAC
TGGTACGTGGATGGCGTGGAGGTGCATAATGCCAAGACAAAGCCGCG
GGAGGAGCAGTTCAACAGCACGTACCGTGTGGTCAGCGTCCTCACCG
TCCTGCACCAGGACTGGCTGAACGGCAAGGAGTACAAGTGCAAGGTC
TCCAACAAAGGCCTCCCGTCCTCCATCGAGAAAACCATCTCCAAAGC
CAAAGGGCAGCCCCGAGAGCCACAGGTGTACACCCTGCCCCCATCCC
AGGAGGAGATGACCAAGAACCAGGTCAGCCTGACCTGCCTGGTCAAA
GGCTTCTACCCCAGCGACATCGCCGTGGAGTGGGAGAGCAATGGGCA
GCCGGAGAACAACTACAAGACCACGCCTCCCGTGCTGGACTCCGACG
GCTCCTTCTTCCTCTACAGCAGGCTCACCGTGGACAAGAGCAGGTGG
CAGGAGGGGAATGTCTTCTCATGCTCCGTGATGCATGAGGCTCTGCA
CAACCACTACACACAGAAGTCCCTCTCCCTGTCTCTGGGTAAATGA
HC Amino Acid Sequence
(SEQ ID NO: 167)
QVQLVQSGAEVKKPGASVKVSCKASGYAFTTYGITWVRQAPGQGLEW
MGWISAYNGNTNYAEKVQGRFTMTTDTSTNTAYMELRSLRSDDTAVY
FCARKGHYGSGTYYNPFGFDFWGQGTMVTVSSASTKGPSVFPLAPCS
RSTSESTAALGCLVKDYFPEPVTVSWNSGALTSGVHTFPAVLQSSGL
YSLSSVVTVPSSSLGTKTYTCNVDHKPSNTKVDKRVESKYGPPCPPC
PAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVK
GFYPSDIAVEWESNGQPENNYKTTPPVLDSDGSFFLYSRLTVDKSRW
QEGNVFSCSVMHEALHNHYTQKSLSLSLGK
LC DNA Sequence
(SEQ ID NO: 300)
GAAATTGTGTTGACGCAGTCTCCAGGCACCCTGTCTTTGTCTCCAGG
GGAAAGAGCCACCCTCTCCTGCAGGGCCAGTCAGAGTGTTAGCAGCA
GCTACTTAGCCTGGTACCAACAGAAACCTGGCCAGGCTCCCAGGCTC
CTCATCTATGGTGCATCCAGCAGGGCCACTGGCATCCCAGACAGGTT
CAGTGGCAGTGGGTCTGGGACAGACTTCACTCTCACCATCAGCAGAC
TGGAGCCTGAAGATTTTGCTTTGTATTTCTGTCAGCAGTATTATGGC
TCACCTTGGACGTTCGGCCAAGGGACCAAGGTGGAAATCAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 168)
EIVLTQSPGTLSLSPGERATLSCRASQSVSSSYLAWYQQKPGQAPRL
LIYGASSRATGIPDRFSGSGSGTDFTLTISRLEPEDFALYFCQQYYG
SPWTFGQGTKVEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC
REGN14570
HCVR DNA Sequence
(SEQ ID NO: 301)
CAGGTGCAGCTACAGCAGTGGGGCGCAGGACTGTTGAAGCCTTCGGC
GACCCTGTCCCGCACCTGCGCTGTCTATGGTGGGTCCTTCAGTGGTT
ACTACTGGAACTGGATCCGCCAGTCCCCAGGGAAGGGGCTGGAATGG
ATTGGGGAAATCCTTCATAGTGGAAGAACCAACTACAACCCGTCCCT
CAAGAGTCGAGTCACCATATCAGTAGACACGTCCAAGAACCAGTTCT
CCCTGAAGCTGACCTCTGTGACCGCCGCGGACACGGCTGTATATTAC
TGTGCGGGAAGGATAGCAGCTCGTCACGGCTGGTTCGACCCCTGGGG
CCAGGGAACCCTGGTCACCGTCTCCTCA
HCVR Amino Acid Sequence
(SEQ ID NO: 97)
QVQLQQWGAGLLKPSATLSRTCAVYGGSFSGYYWNWIRQSPGKGLEW
IGEILHSGRTNYNPSLKSRVTISVDTSKNQFSLKLTSVTAADTAVYY
CAGRIAARHGWFDPWGQGTLVTVSS
HCDR1 DNA Sequence
(SEQ ID NO: 302)
GGTGGGTCCTTCAGTGGTTACTAC
HCDR1 Amino Acid Sequence
(SEQ ID NO: 98)
GGSFSGYY
HCDR2 DNA Sequence
(SEQ ID NO: 303)
ATCCTTCATAGTGGAAGAACC
HCDR2 Amino Acid Sequence
(SEQ ID NO: 99)
ILHSGRT
HCDR3 DNA Sequence
(SEQ ID NO: 304)
GCGGGAAGGATAGCAGCTCGTCACGGCTGGTTCGACCCC
HCDR3 Amino Acid Sequence
(SEQ ID NO: 100)
AGRIAARHGWFDP
LCVR DNA Sequence
(SEQ ID NO: 305)
GACATCCAGATGACCCAGTCTCCATCTTCCGTGTCTACATCTGTAGG
AGACAGAGTCACCATCTCTTGTCGGGCGAGTCAGGATATTCGCAAGT
GGTTAGCCTGGTATCAACAGAAACCAGGAAAAGCCCCTAAACTCCTG
ATCTATGCTACATCCAGTTTGCAAAGTGGGGTCCCTTCAAGGTTCAG
CGGCAGTGGATCTGGGACAGATTTCACTCTCACCATCAGCAGCCTGC
AGCCTGAGGATTTTGCAACTTACTTTTGTCAACAGGCTAACAGTTTC
CCGTTCACTTTTGGCCAGGGGACCAAGCTGGAGATCAAA
LCVR Amino Acid Sequence
(SEQ ID NO: 101)
DIQMTQSPSSVSTSVGDRVTISCRASQDIRKWLAWYQQKPGKAPKLL
IYATSSLQSGVPSRFSGSGSGTDFTLTISSLQPEDFATYFCQQANSF
PFTFGQGTKLEIK
LCDR1 DNA Sequence
(SEQ ID NO: 306)
CAGGATATTCGCAAGTGG
LCDR1 Amino Acid Sequence
(SEQ ID NO: 102)
QDIRKW
LCDR2 DNA Sequence
(SEQ ID NO: 307)
GCTACATCC
LCDR2 Amino Acid Sequence
(SEQ ID NO: 103)
ATS
LCDR3 DNA Sequence
(SEQ ID NO: 308)
CAACAGGCTAACAGTTTCCCGTTCACT
LCDR3 Amino Acid Sequence
(SEQ ID NO: 104)
QQANSFPFT
HC DNA Sequence
(SEQ ID NO: 309)
CAGGTGCAGCTACAGCAGTGGGGCGCAGGACTGTTGAAGCCTTCGGC
GACCCTGTCCCGCACCTGCGCTGTCTATGGTGGGTCCTTCAGTGGTT
ACTACTGGAACTGGATCCGCCAGTCCCCAGGGAAGGGGCTGGAATGG
ATTGGGGAAATCCTTCATAGTGGAAGAACCAACTACAACCCGTCCCT
CAAGAGTCGAGTCACCATATCAGTAGACACGTCCAAGAACCAGTTCT
CCCTGAAGCTGACCTCTGTGACCGCCGCGGACACGGCTGTATATTAC
TGTGCGGGAAGGATAGCAGCTCGTCACGGCTGGTTCGACCCCTGGGG
CCAGGGAACCCTGGTCACCGTCTCCTCAGCCTCCACCAAGGGCCCAT
CGGTCTTCCCCCTGGCACCCTCCTCCAAGAGCACCTCTGGGGGCACA
GCGGCCCTGGGCTGCCTGGTCAAGGACTACTTCCCCGAACCGGTGAC
GGTGTCGTGGAACTCAGGCGCCCTGACCAGCGGCGTGCACACCTTCC
CGGCTGTCCTACAGTCCTCAGGACTCTACTCCCTCAGCAGCGTGGTG
ACCGTGCCCTCCAGCAGCTTGGGCACCCAGACCTACATCTGCAACGT
GAATCACAAGCCCAGCAACACCAAGGTGGACAAGAAAGTTGAGCCCA
AATCTTGTGACAAAACTCACACATGCCCACCGTGCCCAGCACCTGAA
CTCCTGGGGGGACCGTCAGTCTTCCTCTTCCCCCCAAAACCCAAGGA
CACCCTCATGATCTCCCGGACCCCTGAGGTCACATGCGTGGTGGTGG
ACGTGAGCCACGAAGACCCTGAGGTCAAGTTCAACTGGTACGTGGAC
GGCGTGGAGGTGCATAATGCCAAGACAAAGCCGCGGGAGGAGCAGTA
CCAAAGCACGTACCGTGTGGTCAGCGTCCTCACCGTCCTGCACCAGG
ACTGGCTGAATGGCAAGGAGTACAAGTGCAAGGTCTCCAACAAAGCC
CTCCCAGCCCCCATCGAGAAAACCATCTCCAAAGCCAAAGGGCAGCC
CCGAGAACCACAGGTGTACACCCTGCCCCCATCCCGGGATGAGCTGA
CCAAGAACCAGGTCAGCCTGACCTGCCTGGTCAAAGGCTTCTATCCC
AGCGACATCGCCGTGGAGTGGGAGAGCAATGGGCAGCCGGAGAACAA
CTACAAGACCACGCCTCCCGTGCTGGACTCCGACGGCTCCTTCTTCC
TCTACAGCAAGCTCACCGTGGACAAGAGCAGGTGGCAGCAGGGGAAC
GTCTTCTCATGCTCCGTGATGCATGAGGCTCTGCACAACCACTACAC
GCAGAAGTCCCTCTCCCTGTCTCCGGGTAAATGA
HC Amino Acid Sequence
(N297Q is indicated in bold)
(SEQ ID NO: 169)
QVQLQQWGAGLLKPSATLSRTCAVYGGSFSGYYWNWIRQSPGKGLEW
IGEILHSGRTNYNPSLKSRVTISVDTSKNQFSLKLTSVTAADTAVYY
CAGRIAARHGWFDPWGQGTLVTVSSASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYQSTYRVVSVLTVLHQDWLNGKEYKCKVSNKA
LPAPIEKTISKAKGQPREPQVYTLPPSRDELTKNQVSLTCLVKGFYP
SDIAVEWESNGQPENNYKTTPPVLDSDGSFFLYSKLTVDKSRWQQGN
VFSCSVMHEALHNHYTQKSLSLSPGK
LC DNA Sequence
(SEQ ID NO: 310)
GACATCCAGATGACCCAGTCTCCATCTTCCGTGTCTACATCTGTAGG
AGACAGAGTCACCATCTCTTGTCGGGCGAGTCAGGATATTCGCAAGT
GGTTAGCCTGGTATCAACAGAAACCAGGAAAAGCCCCTAAACTCCTG
ATCTATGCTACATCCAGTTTGCAAAGTGGGGTCCCTTCAAGGTTCAG
CGGCAGTGGATCTGGGACAGATTTCACTCTCACCATCAGCAGCCTGC
AGCCTGAGGATTTTGCAACTTACTTTTGTCAACAGGCTAACAGTTTC
CCGTTCACTTTTGGCCAGGGGACCAAGCTGGAGATCAAACGAACTGT
GGCTGCACCATCTGTCTTCATCTTCCCGCCATCTGATGAGCAGTTGA
AATCTGGAACTGCCTCTGTTGTGTGCCTGCTGAATAACTTCTATCCC
AGAGAGGCCAAAGTACAGTGGAAGGTGGATAACGCCCTCCAATCGGG
TAACTCCCAGGAGAGTGTCACAGAGCAGGACAGCAAGGACAGCACCT
ACAGCCTCAGCAGCACCCTGACGCTGAGCAAAGCAGACTACGAGAAA
CACAAAGTCTACGCCTGCGAAGTCACCCATCAGGGCCTGAGCTCGCC
CGTCACAAAGAGCTTCAACAGGGGAGAGTGTTAG
LC Amino Acid Sequence
(SEQ ID NO: 170)
DIQMTQSPSSVSTSVGDRVTISCRASQDIRKWLAWYQQKPGKAPKLL
IYATSSLQSGVPSRFSGSGSGTDFTLTISSLQPEDFATYFCQQANSF
PFTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
REGN14571
HCVR DNA Sequence
(SEQ ID NO: 311)
CAGGTGCAGCTGCAGGAGTCGGGCCCAGGACTGGTGAAGCCTTCGGA
GACCCTGTCCCTCACCTGCACTGTCTCTGGTGACTCCATCAATAATT
ACTACTGGACCTGGCTCCGGCAGCCCCCAGGGAAGGGACTGGAGTGG
ATTGGTTATATCTATTACAGTGGGAGCGCCAACTACAACCCCTCCCT
CAAGAGTCGAGTCACCATATCAGTAGACACGTCCAAGAACCAGTTCT
CCCTGAAGCTAAATTCTGTGACCGCTGCGGACACGGCCGTGTATTAC
TGTGCGAGAGGGGCGGTCAAGTACTTCCGGCATTGGGGCCAGGGCAC
CCTGGTCACCGTCTCCTCA
HCVR Amino Acid Sequence
(SEQ ID NO: 105)
QVQLQESGPGLVKPSETLSLTCTVSGDSINNYYWTWLRQPPGKGLEW
IGYIYYSGSANYNPSLKSRVTISVDTSKNQFSLKLNSVTAADTAVYY
CARGAVKYFRHWGQGTLVTVSS
HCDR1 DNA Sequence
(SEQ ID NO: 312)
GGTGACTCCATCAATAATTACTAC
HCDR1 Amino Acid Sequence
(SEQ ID NO: 106)
GDSINNYY
HCDR2 DNA Sequence
(SEQ ID NO: 313)
ATCTATTACAGTGGGAGCGCC
HCDR2 Amino Acid Sequence
(SEQ ID NO: 107)
IYYSGSA
HCDR3 DNA Sequence
(SEQ ID NO: 314)
GCGAGAGGGGCGGTCAAGTACTTCCGGCAT
HCDR3 Amino Acid Sequence
ARGAVKYFRH
(SEQ ID NO: 108)
LCVR DNA Sequence
(SEQ ID NO: 315)
GAAATTGTGTTGACGCAGTCTCCGGGCACCCTCTCTTTGTCTCCAGG
GGAAAGAGCCACCCTCTCCTGCAGGGCCAGTCAGACTATTAACCACA
ACAACTTAGCCTGGTACCAGCAGAGACCTGGCCAGGCTCCCAGGCTC
CTCATCTATGGTGCATCCAACAGGGCCACTGCCATCCCAGACAGGTT
CAGTGGCAGTGGGTCTGGGACAGACTTCACTCTCACCATCAGCAGAC
TGGAGCCTGAAGATTTTGAAGTGTATTCTTGTCAGCAGTATGGTAGC
TTGCCGCTCACTTTCGGCGGAGGGACCAAGGTGGAGATCAAA
LCVR Amino Acid Sequence
(SEQ ID NO: 109)
EIVLTQSPGTLSLSPGERATLSCRASQTINHNNLAWYQQRPGQAPRL
LIYGASNRATAIPDRFSGSGSGTDFTLTISRLEPEDFEVYSCQQYGS
LPLTFGGGTKVEIK
LCDR1 DNA Sequence
(SEQ ID NO: 316)
CAGACTATTAACCACAACAAC
LCDR1 Amino Acid Sequence
(SEQ ID NO: 110)
QTINHNN
LCDR2 DNA Sequence
(SEQ ID NO: 317)
GGTGCATCC
LCDR2 Amino Acid Sequence
(SEQ ID NO: 111)
GAS
LCDR3 DNA Sequence
(SEQ ID NO: 318)
CAGCAGTATGGTAGCTTGCCGCTCACT
LCDR3 Amino Acid Sequence
(SEQ ID NO: 112)
QQYGSLPLT
HC DNA Sequence
(SEQ ID NO: 319)
CAGGTGCAGCTGCAGGAGTCGGGCCCAGGACTGGTGAAGCCTTCGGA
GACCCTGTCCCTCACCTGCACTGTCTCTGGTGACTCCATCAATAATT
ACTACTGGACCTGGCTCCGGCAGCCCCCAGGGAAGGGACTGGAGTGG
ATTGGTTATATCTATTACAGTGGGAGCGCCAACTACAACCCCTCCCT
CAAGAGTCGAGTCACCATATCAGTAGACACGTCCAAGAACCAGTTCT
CCCTGAAGCTAAATTCTGTGACCGCTGCGGACACGGCCGTGTATTAC
TGTGCGAGAGGGGCGGTCAAGTACTTCCGGCATTGGGGCCAGGGCAC
CCTGGTCACCGTCTCCTCAGCCTCCACCAAGGGCCCATCGGTCTTCC
CCCTGGCACCCTCCTCCAAGAGCACCTCTGGGGGCACAGCGGCCCTG
GGCTGCCTGGTCAAGGACTACTTCCCCGAACCGGTGACGGTGTCGTG
GAACTCAGGCGCCCTGACCAGCGGCGTGCACACCTTCCCGGCTGTCC
TACAGTCCTCAGGACTCTACTCCCTCAGCAGCGTGGTGACCGTGCCC
TCCAGCAGCTTGGGCACCCAGACCTACATCTGCAACGTGAATCACAA
GCCCAGCAACACCAAGGTGGACAAGAAAGTTGAGCCCAAATCTTGTG
ACAAAACTCACACATGCCCACCGTGCCCAGCACCTGAACTCCTGGGG
GGACCGTCAGTCTTCCTCTTCCCCCCAAAACCCAAGGACACCCTCAT
GATCTCCCGGACCCCTGAGGTCACATGCGTGGTGGTGGACGTGAGCC
ACGAAGACCCTGAGGTCAAGTTCAACTGGTACGTGGACGGCGTGGAG
GTGCATAATGCCAAGACAAAGCCGCGGGAGGAGCAGTACCAAAGCAC
GTACCGTGTGGTCAGCGTCCTCACCGTCCTGCACCAGGACTGGCTGA
ATGGCAAGGAGTACAAGTGCAAGGTCTCCAACAAAGCCCTCCCAGCC
CCCATCGAGAAAACCATCTCCAAAGCCAAAGGGCAGCCCCGAGAACC
ACAGGTGTACACCCTGCCCCCATCCCGGGATGAGCTGACCAAGAACC
AGGTCAGCCTGACCTGCCTGGTCAAAGGCTTCTATCCCAGCGACATC
GCCGTGGAGTGGGAGAGCAATGGGCAGCCGGAGAACAACTACAAGAC
CACGCCTCCCGTGCTGGACTCCGACGGCTCCTTCTTCCTCTACAGCA
AGCTCACCGTGGACAAGAGCAGGTGGCAGCAGGGGAACGTCTTCTCA
TGCTCCGTGATGCATGAGGCTCTGCACAACCACTACACGCAGAAGTC
CCTCTCCCTGTCTCCGGGTAAATGA
HC Amino Acid Sequence
(N297Q is indicated in bold)
(SEQ ID NO: 171)
QVQLQESGPGLVKPSETLSLTCTVSGDSINNYYWTWLRQPPGKGLEW
IGYIYYSGSANYNPSLKSRVTISVDTSKNQFSLKLNSVTAADTAVYY
CARGAVKYFRHWGQGTLVTVSSASTKGPSVFPLAPSSKSTSGGTAAL
GCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVE
VHNAKTKPREEQYQSTYRVVSVLTVLHQDWLNGKEYKCKVSNKALPA
PIEKTISKAKGQPREPQVYTLPPSRDELTKNQVSLTCLVKGFYPSDI
AVEWESNGQPENNYKTTPPVLDSDGSFFLYSKLTVDKSRWQQGNVFS
CSVMHEALHNHYTQKSLSLSPGK
LC DNA Sequence
(SEQ ID NO: 320)
GAAATTGTGTTGACGCAGTCTCCGGGCACCCTCTCTTTGTCTCCAGG
GGAAAGAGCCACCCTCTCCTGCAGGGCCAGTCAGACTATTAACCACA
ACAACTTAGCCTGGTACCAGCAGAGACCTGGCCAGGCTCCCAGGCTC
CTCATCTATGGTGCATCCAACAGGGCCACTGCCATCCCAGACAGGTT
CAGTGGCAGTGGGTCTGGGACAGACTTCACTCTCACCATCAGCAGAC
TGGAGCCTGAAGATTTTGAAGTGTATTCTTGTCAGCAGTATGGTAGC
TTGCCGCTCACTTTCGGCGGAGGGACCAAGGTGGAGATCAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 172)
EIVLTQSPGTLSLSPGERATLSCRASQTINHNNLAWYQQRPGQAPRL
LIYGASNRATAIPDRFSGSGSGTDFTLTISRLEPEDFEVYSCQQYGS
LPLTFGGGTKVEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC
REGN14572
HCVR DNA Sequence
(SEQ ID NO: 321)
CAGGTGCAGCTGGTGGAGTCTGGGGGAGGCGTGGTCCAGCCTGGGAG
GTCCCTGAGACTCTCCTGTGCAGCGTCTGGATTCACCTTCAGTACAT
ATGGCATGCACTGGGTCCGCCAGGCTCCAGGCAAGGGGCTGGAGTGG
GTGGCAGTTATTTGGCATGATGGAAGTGATAAATATTATGTAGACTC
CGTGAAGGGCCGATTCTCCATCGCCAGAGACAATTCCAAGAACACGC
TTTATCTGCAAATGAATAGTCTGAGAGTCGAGGACACGGGTATATAT
TACTGTGCGAGAAGGGGTATACGTGGAACCGTTTTTGACCACTGGGG
CCTGGGAACCCTGGTCACCGTCTCCTCA
HCVR Amino Acid Sequence
(SEQ ID NO: 113)
QVQLVESGGGVVQPGRSLRLSCAASGFTFSTYGMHWVRQAPGKGLEW
VAVIWHDGSDKYYVDSVKGRFSIARDNSKNTLYLQMNSLRVEDTGIY
YCARRGIRGTVFDHWGLGTLVTVSS
HCDR1 DNA Sequence
(SEQ ID NO: 322)
GGATTCACCTTCAGTACATATGGC
HCDR1 Amino Acid Sequence
(SEQ ID NO: 114)
GFTFSTYG
HCDR2 DNA Sequence
(SEQ ID NO: 323)
ATTTGGCATGATGGAAGTGATAAA
HCDR2 Amino Acid Sequence
(SEQ ID NO: 115)
IWHDGSDK
HCDR3 DNA Sequence
(SEQ ID NO: 324)
GCGAGAAGGGGTATACGTGGAACCGTTTTTGACCAC
HCDR3 Amino Acid Sequence
(SEQ ID NO: 116)
ARRGIRGTVFDH
LCVR DNA Sequence
(SEQ ID NO: 325)
GACATCCAGATGACCCAGTCTCCTTCCACCCTGTCTGCATCTGTAGG
AGACAGAGTCACCCTCACTTGTCGGGCCAGTCAGAGTATTAGTAACA
AGTTGGCCTGGTATCAGCAGAAACCAGGGAAAGCCCCTAACCTCCTG
ATCTATAAGGCGTCTAATTTAGAAAGTGGGGTCCCATCAAGGTTCAG
CGGCAGTGGATCTGGGACAGAATTCACTCTCACCATCAGCAGCCTGC
AGCCTGATGATTTTGCAACTTATTACTGCCAACAGTATAATAGTTAT
TCGTGGACGTTCGGCCAAGGGACCAAGGTGGAAATCAAA
LCVR Amino Acid Sequence
(SEQ ID NO: 117)
DIQMTQSPSTLSASVGDRVTLTCRASQSISNKLAWYQQKPGKAPNLL
IYKASNLESGVPSRFSGSGSGTEFTLTISSLQPDDFATYYCQQYNSY
SWTFGQGTKVEIK
LCDR1 DNA Sequence
(SEQ ID NO: 326)
CAGAGTATTAGTAACAAG
LCDR1 Amino Acid Sequence
(SEQ ID NO: 118)
QSISNK
LCDR2 DNA Sequence
(SEQ ID NO: 327)
AAGGCGTCT
LCDR2 Amino Acid Sequence
(SEQ ID NO: 119)
KAS
LCDR3 DNA Sequence
(SEQ ID NO: 328)
CAACAGTATAATAGTTATTCGTGGACG
LCDR3 Amino Acid Sequence
(SEQ ID NO: 120)
QQYNSYSWT
HC DNA Sequence
(SEQ ID NO: 329)
CAGGTGCAGCTGGTGGAGTCTGGGGGAGGCGTGGTCCAGCCTGGGAG
GTCCCTGAGACTCTCCTGTGCAGCGTCTGGATTCACCTTCAGTACAT
ATGGCATGCACTGGGTCCGCCAGGCTCCAGGCAAGGGGCTGGAGTGG
GTGGCAGTTATTTGGCATGATGGAAGTGATAAATATTATGTAGACTC
CGTGAAGGGCCGATTCTCCATCGCCAGAGACAATTCCAAGAACACGC
TTTATCTGCAAATGAATAGTCTGAGAGTCGAGGACACGGGTATATAT
TACTGTGCGAGAAGGGGTATACGTGGAACCGTTTTTGACCACTGGGG
CCTGGGAACCCTGGTCACCGTCTCCTCAGCCTCCACCAAGGGCCCAT
CGGTCTTCCCCCTGGCACCCTCCTCCAAGAGCACCTCTGGGGGCACA
GCGGCCCTGGGCTGCCTGGTCAAGGACTACTTCCCCGAACCGGTGAC
GGTGTCGTGGAACTCAGGCGCCCTGACCAGCGGCGTGCACACCTTCC
CGGCTGTCCTACAGTCCTCAGGACTCTACTCCCTCAGCAGCGTGGTG
ACCGTGCCCTCCAGCAGCTTGGGCACCCAGACCTACATCTGCAACGT
GAATCACAAGCCCAGCAACACCAAGGTGGACAAGAAAGTTGAGCCCA
AATCTTGTGACAAAACTCACACATGCCCACCGTGCCCAGCACCTGAA
CTCCTGGGGGGACCGTCAGTCTTCCTCTTCCCCCCAAAACCCAAGGA
CACCCTCATGATCTCCCGGACCCCTGAGGTCACATGCGTGGTGGTGG
ACGTGAGCCACGAAGACCCTGAGGTCAAGTTCAACTGGTACGTGGAC
GGCGTGGAGGTGCATAATGCCAAGACAAAGCCGCGGGAGGAGCAGTA
CCAAAGCACGTACCGTGTGGTCAGCGTCCTCACCGTCCTGCACCAGG
ACTGGCTGAATGGCAAGGAGTACAAGTGCAAGGTCTCCAACAAAGCC
CTCCCAGCCCCCATCGAGAAAACCATCTCCAAAGCCAAAGGGCAGCC
CCGAGAACCACAGGTGTACACCCTGCCCCCATCCCGGGATGAGCTGA
CCAAGAACCAGGTCAGCCTGACCTGCCTGGTCAAAGGCTTCTATCCC
AGCGACATCGCCGTGGAGTGGGAGAGCAATGGGCAGCCGGAGAACAA
CTACAAGACCACGCCTCCCGTGCTGGACTCCGACGGCTCCTTCTTCC
TCTACAGCAAGCTCACCGTGGACAAGAGCAGGTGGCAGCAGGGGAAC
GTCTTCTCATGCTCCGTGATGCATGAGGCTCTGCACAACCACTACAC
GCAGAAGTCCCTCTCCCTGTCTCCGGGTAAATGA
HC Amino Acid Sequence
(N297Q is indicated in bold)
(SEQ ID NO: 173)
QVQLVESGGGVVQPGRSLRLSCAASGFTFSTYGMHWVRQAPGKGLEW
VAVIWHDGSDKYYVDSVKGRFSIARDNSKNTLYLQMNSLRVEDTGIY
YCARRGIRGTVFDHWGLGTLVTVSSASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYQSTYRVVSVLTVLHQDWLNGKEYKCKVSNKA
LPAPIEKTISKAKGQPREPQVYTLPPSRDELTKNQVSLTCLVKGFYP
SDIAVEWESNGQPENNYKTTPPVLDSDGSFFLYSKLTVDKSRWQQGN
VFSCSVMHEALHNHYTQKSLSLSPGK
LC DNA Sequence
(SEQ ID NO: 330)
GACATCCAGATGACCCAGTCTCCTTCCACCCTGTCTGCATCTGTAGG
AGACAGAGTCACCCTCACTTGTCGGGCCAGTCAGAGTATTAGTAACA
AGTTGGCCTGGTATCAGCAGAAACCAGGGAAAGCCCCTAACCTCCTG
ATCTATAAGGCGTCTAATTTAGAAAGTGGGGTCCCATCAAGGTTCAG
CGGCAGTGGATCTGGGACAGAATTCACTCTCACCATCAGCAGCCTGC
AGCCTGATGATTTTGCAACTTATTACTGCCAACAGTATAATAGTTAT
TCGTGGACGTTCGGCCAAGGGACCAAGGTGGAAATCAAACGAACTGT
GGCTGCACCATCTGTCTTCATCTTCCCGCCATCTGATGAGCAGTTGA
AATCTGGAACTGCCTCTGTTGTGTGCCTGCTGAATAACTTCTATCCC
AGAGAGGCCAAAGTACAGTGGAAGGTGGATAACGCCCTCCAATCGGG
TAACTCCCAGGAGAGTGTCACAGAGCAGGACAGCAAGGACAGCACCT
ACAGCCTCAGCAGCACCCTGACGCTGAGCAAAGCAGACTACGAGAAA
CACAAAGTCTACGCCTGCGAAGTCACCCATCAGGGCCTGAGCTCGCC
CGTCACAAAGAGCTTCAACAGGGGAGAGTGTTAG
LC Amino Acid Sequence
(SEQ ID NO: 174)
DIQMTQSPSTLSASVGDRVTLTCRASQSISNKLAWYQQKPGKAPNLL
IYKASNLESGVPSRFSGSGSGTEFTLTISSLQPDDFATYYCQQYNSY
SWTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
REGN14573
HCVR DNA Sequence
(SEQ ID NO: 331)
GAGGTGCAGCTGGTGGAGTCTGGGGGAAACTTGGTACAGCCTGGGGG
GTCCCTGAGACTCTCCTGTGCAGCCTCTGGATTCACCTTTACCAGCC
ATGCCATGAACTGGGTCCGCCAGGCTCCAGGGAAGGGGCTGGAGTGG
GTCTCAGTTATTACTGGTAGAGGTTTTGACACACACTACGCTGACTC
CGTGAAGGGCCGGTTCACCATCTCCAGAGACATTTCCAAAAACACGC
TGTATCTGCAAATGAACAGCCTGAGAGCCGAGGACACGGCCGTTTAT
TACTGTGCGAAAGGTCTCTATGATTCGGGGAATTATTATATCGATTA
CTGGGGCCAGGGAACCCTGGTCACCGTCTCCTCA
HCVR Amino Acid Sequence
(SEQ ID NO: 121)
EVQLVESGGNLVQPGGSLRLSCAASGFTFTSHAMNWVRQAPGKGLEW
VSVITGRGFDTHYADSVKGRFTISRDISKNTLYLQMNSLRAEDTAVY
YCAKGLYDSGNYYIDYWGQGTLVTVSS
HCDR1 DNA Sequence
(SEQ ID NO: 332)
GGATTCACCTTTACCAGCCATGCC
HCDR1 Amino Acid Sequence
(SEQ ID NO: 122)
GFTFTSHA
HCDR2 DNA Sequence
(SEQ ID NO: 333)
ATTACTGGTAGAGGTTTTGACACA
HCDR2 Amino Acid Sequence
(SEQ ID NO: 123)
ITGRGFDT
HCDR3 DNA Sequence
(SEQ ID NO: 334)
GCGAAAGGTCTCTATGATTCGGGGAATTATTATATCGATTAC
HCDR3 Amino Acid Sequence
(SEQ ID NO: 124)
AKGLYDSGNYYIDY
LCVR DNA Sequence
(SEQ ID NO: 335)
CAGTCTGTGTTGACGCAGCCGCCCTCAGTGTCTGCGGCCCCAGGACA
GAAGGTCACCATCTCCTGCTCTGGAAGCAGCTCCAACATTGGGAATA
ATTATGTTTCCTGGTACCAGCAGCTCCCAGGAACAGCCCCCAAACTC
CTCATTTATGACAATAATAAGCGACCCTCAGGGATTCCTGACCGATT
CTCTGGCTCCAAGTCTGGCACGTCAGCCACCCTGGGCATCACCGGAC
TCCAGACTGGGGACGAGGCCGATTATTACTGCGGAACATGGGATCTC
AGCCTGAGTTTCAATTGGGTGTTCGGCGGAGGGACCAAGCTGACCGT
CCTA
LCVR Amino Acid Sequence
(SEQ ID NO: 125)
QSVLTQPPSVSAAPGQKVTISCSGSSSNIGNNYVSWYQQLPGTAPKL
LIYDNNKRPSGIPDRFSGSKSGTSATLGITGLQTGDEADYYCGTWDL
SLSFNWVFGGGTKLTVL
LCDR1 DNA Sequence
(SEQ ID NO: 336)
AGCTCCAACATTGGGAATAATTAT
LCDR1 Amino Acid Sequence
(SEQ ID NO: 126)
SSNIGNNY
CDR2 DNA Sequence
(SEQ ID NO: 337)
GACAATAAT
LCDR2 Amino Acid Sequence
(SEQ ID NO: 127)
DNN
LCDR3 DNA Sequence
(SEQ ID NO: 338)
GGAACATGGGATCTCAGCCTGAGTTTCAATTGGGTG
LCDR3 Amino Acid Sequence
(SEQ ID NO: 128)
GTWDLSLSFNWV
HC DNA Sequence
(SEQ ID NO: 339)
GAGGTGCAGCTGGTGGAGTCTGGGGGAAACTTGGTACAGCCTGGGGG
GTCCCTGAGACTCTCCTGTGCAGCCTCTGGATTCACCTTTACCAGCC
ATGCCATGAACTGGGTCCGCCAGGCTCCAGGGAAGGGGCTGGAGTGG
GTCTCAGTTATTACTGGTAGAGGTTTTGACACACACTACGCTGACTC
CGTGAAGGGCCGGTTCACCATCTCCAGAGACATTTCCAAAAACACGC
TGTATCTGCAAATGAACAGCCTGAGAGCCGAGGACACGGCCGTTTAT
TACTGTGCGAAAGGTCTCTATGATTCGGGGAATTATTATATCGATTA
CTGGGGCCAGGGAACCCTGGTCACCGTCTCCTCAGCCTCCACCAAGG
GCCCATCGGTCTTCCCCCTGGCACCCTCCTCCAAGAGCACCTCTGGG
GGCACAGCGGCCCTGGGCTGCCTGGTCAAGGACTACTTCCCCGAACC
GGTGACGGTGTCGTGGAACTCAGGCGCCCTGACCAGCGGCGTGCACA
CCTTCCCGGCTGTCCTACAGTCCTCAGGACTCTACTCCCTCAGCAGC
GTGGTGACCGTGCCCTCCAGCAGCTTGGGCACCCAGACCTACATCTG
CAACGTGAATCACAAGCCCAGCAACACCAAGGTGGACAAGAAAGTTG
AGCCCAAATCTTGTGACAAAACTCACACATGCCCACCGTGCCCAGCA
CCTGAACTCCTGGGGGGACCGTCAGTCTTCCTCTTCCCCCCAAAACC
CAAGGACACCCTCATGATCTCCCGGACCCCTGAGGTCACATGCGTGG
TGGTGGACGTGAGCCACGAAGACCCTGAGGTCAAGTTCAACTGGTAC
GTGGACGGCGTGGAGGTGCATAATGCCAAGACAAAGCCGCGGGAGGA
GCAGTACCAAAGCACGTACCGTGTGGTCAGCGTCCTCACCGTCCTGC
ACCAGGACTGGCTGAATGGCAAGGAGTACAAGTGCAAGGTCTCCAAC
AAAGCCCTCCCAGCCCCCATCGAGAAAACCATCTCCAAAGCCAAAGG
GCAGCCCCGAGAACCACAGGTGTACACCCTGCCCCCATCCCGGGATG
AGCTGACCAAGAACCAGGTCAGCCTGACCTGCCTGGTCAAAGGCTTC
TATCCCAGCGACATCGCCGTGGAGTGGGAGAGCAATGGGCAGCCGGA
GAACAACTACAAGACCACGCCTCCCGTGCTGGACTCCGACGGCTCCT
TCTTCCTCTACAGCAAGCTCACCGTGGACAAGAGCAGGTGGCAGCAG
GGGAACGTCTTCTCATGCTCCGTGATGCATGAGGCTCTGCACAACCA
CTACACGCAGAAGTCCCTCTCCCTGTCTCCGGGTAAATGA
HC Amino Acid Sequence
(N297Q is indicated in bold)
(SEQ ID NO: 175)
EVQLVESGGNLVQPGGSLRLSCAASGFTFTSHAMNWVRQAPGKGLEW
VSVITGRGFDTHYADSVKGRFTISRDISKNTLYLQMNSLRAEDTAVY
YCAKGLYDSGNYYIDYWGQGTLVTVSSASTKGPSVFPLAPSSKSTSG
GTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWY
VDGVEVHNAKTKPREEQYQSTYRVVSVLTVLHQDWLNGKEYKCKVSN
KALPAPIEKTISKAKGQPREPQVYTLPPSRDELTKNQVSLTCLVKGF
YPSDIAVEWESNGQPENNYKTTPPVLDSDGSFFLYSKLTVDKSRWQQ
GNVFSCSVMHEALHNHYTQKSLSLSPGK
LC DNA Sequence
(SEQ ID NO: 340)
CAGTCTGTGTTGACGCAGCCGCCCTCAGTGTCTGCGGCCCCAGGACA
GAAGGTCACCATCTCCTGCTCTGGAAGCAGCTCCAACATTGGGAATA
ATTATGTTTCCTGGTACCAGCAGCTCCCAGGAACAGCCCCCAAACTC
CTCATTTATGACAATAATAAGCGACCCTCAGGGATTCCTGACCGATT
CTCTGGCTCCAAGTCTGGCACGTCAGCCACCCTGGGCATCACCGGAC
TCCAGACTGGGGACGAGGCCGATTATTACTGCGGAACATGGGATCTC
AGCCTGAGTTTCAATTGGGTGTTCGGCGGAGGGACCAAGCTGACCGT
CCTAGGCCAGCCCAAGGCCGCCCCCTCCGTGACCCTGTTCCCCCCCT
CCTCCGAGGAGCTGCAGGCCAACAAGGCCACCCTGGTGTGCCTGATC
TCCGACTTCTACCCCGGCGCCGTGACCGTGGCCTGGAAGGCCGACTC
CTCCCCCGTGAAGGCCGGCGTGGAGACCACCACCCCCTCCAAGCAGT
CCAACAACAAGTACGCCGCCTCCTCCTACCTGTCCCTGACCCCCGAG
CAGTGGAAGTCCCACCGGTCCTACTCCTGCCAGGTGACCCACGAGGG
CTCCACCGTGGAGAAGACCGTGGCCCCCACCGAGTGCTCCTGA
LC Amino Acid Sequence
(SEQ ID NO: 176)
QSVLTQPPSVSAAPGQKVTISCSGSSSNIGNNYVSWYQQLPGTAPKL
LIYDNNKRPSGIPDRFSGSKSGTSATLGITGLQTGDEADYYCGTWDL
SLSFNWVFGGGTKLTVLGQPKAAPSVTLFPPSSEELQANKATLVCLI
SDFYPGAVTVAWKADSSPVKAGVETTTPSKQSNNKYAASSYLSLTPE
QWKSHRSYSCQVTHEGSTVEKTVAPTECS
REGN14574
HCVR DNA Sequence
(SEQ ID NO: 341)
CAGGTGCAGCTGGTGGAGTCTGGGGGAGGCGTGGTCCAGCCTGGGAC
GTCCCTGAGACTCTCCTGTGCAGCGTCAGGATTCACCTTCAGTAGCT
ATGGCATGCACTGGGTCCGCCAGGCTCCAGGCAAGGGGCTGGAGTGG
GTGGCAGTTATATGGATTGATGGAAGTAATAAATATTATGCAGACTC
CGTGAAGGGCCGATTCACCATCTCCAGAGACAATTCCAAGAACACGC
TGTATCTGCAAATGAACAGCCTGAGAGCCGAGGACACGGCTGTGTAT
TACTGTGCGAGAAGGGGGGGTATAGTAGTAGCTGCCCCCTTTGACTA
CTGGGGCCAGGGAACCCTGGTCACCGTCTCCTCA
HCVR Amino Acid Sequence
(SEQ ID NO: 129)
QVQLVESGGGVVQPGTSLRLSCAASGFTFSSYGMHWVRQAPGKGLEW
VAVIWIDGSNKYYADSVKGRFTISRDNSKNTLYLQMNSLRAEDTAVY
YCARRGGIVVAAPFDYWGQGTLVTVSS
HCDR1 DNA Sequence
(SEQ ID NO: 342)
GGATTCACCTTCAGTAGCTATGGC
HCDR1 Amino Acid Sequence
(SEQ ID NO: 130)
GFTFSSYG
HCDR2 DNA Sequence
(SEQ ID NO: 343)
ATATGGATTGATGGAAGTAATAAA
HCDR2 Amino Acid Sequence
(SEQ ID NO: 131)
IWIDGSNK
HCDR3 DNA Sequence
(SEQ ID NO: 344)
GCGAGAAGGGGGGGTATAGTAGTAGCTGCCCCCTTTGACTAC
HCDR3 Amino Acid Sequence
(SEQ ID NO: 132)
ARRGGIVVAAPFDY
LCVR DNA Sequence
(SEQ ID NO: 345)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAA
LCVR Amino Acid Sequence
(SEQ ID NO: 133)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIK
LCDR1 DNA Sequence
(SEQ ID NO: 346)
CAGAGCATTAGCAGCTAT
LCDR1 Amino Acid Sequence
(SEQ ID NO: 134)
QSISSY
LCDR2 DNA Sequence
(SEQ ID NO: 347)
GCTGCATCC
LCDR2 Amino Acid Sequence
(SEQ ID NO: 135)
AAS
LCDR3 DNA Sequence
(SEQ ID NO: 348)
CAACAGAGTTACAGTACCCCTCCGATCACC
LCDR3 Amino Acid Sequence
(SEQ ID NO: 136)
QQSYSTPPIT
HC DNA Sequence
(SEQ ID NO: 349)
CAGGTGCAGCTGGTGGAGTCTGGGGGAGGCGTGGTCCAGCCTGGGAC
GTCCCTGAGACTCTCCTGTGCAGCGTCAGGATTCACCTTCAGTAGCT
ATGGCATGCACTGGGTCCGCCAGGCTCCAGGCAAGGGGCTGGAGTGG
GTGGCAGTTATATGGATTGATGGAAGTAATAAATATTATGCAGACTC
CGTGAAGGGCCGATTCACCATCTCCAGAGACAATTCCAAGAACACGC
TGTATCTGCAAATGAACAGCCTGAGAGCCGAGGACACGGCTGTGTAT
TACTGTGCGAGAAGGGGGGGTATAGTAGTAGCTGCCCCCTTTGACTA
CTGGGGCCAGGGAACCCTGGTCACCGTCTCCTCAGCCTCCACCAAGG
GCCCATCGGTCTTCCCCCTGGCACCCTCCTCCAAGAGCACCTCTGGG
GGCACAGCGGCCCTGGGCTGCCTGGTCAAGGACTACTTCCCCGAACC
GGTGACGGTGTCGTGGAACTCAGGCGCCCTGACCAGCGGCGTGCACA
CCTTCCCGGCTGTCCTACAGTCCTCAGGACTCTACTCCCTCAGCAGC
GTGGTGACCGTGCCCTCCAGCAGCTTGGGCACCCAGACCTACATCTG
CAACGTGAATCACAAGCCCAGCAACACCAAGGTGGACAAGAAAGTTG
AGCCCAAATCTTGTGACAAAACTCACACATGCCCACCGTGCCCAGCA
CCTGAACTCCTGGGGGGACCGTCAGTCTTCCTCTTCCCCCCAAAACC
CAAGGACACCCTCATGATCTCCCGGACCCCTGAGGTCACATGCGTGG
TGGTGGACGTGAGCCACGAAGACCCTGAGGTCAAGTTCAACTGGTAC
GTGGACGGCGTGGAGGTGCATAATGCCAAGACAAAGCCGCGGGAGGA
GCAGTACCAAAGCACGTACCGTGTGGTCAGCGTCCTCACCGTCCTGC
ACCAGGACTGGCTGAATGGCAAGGAGTACAAGTGCAAGGTCTCCAAC
AAAGCCCTCCCAGCCCCCATCGAGAAAACCATCTCCAAAGCCAAAGG
GCAGCCCCGAGAACCACAGGTGTACACCCTGCCCCCATCCCGGGATG
AGCTGACCAAGAACCAGGTCAGCCTGACCTGCCTGGTCAAAGGCTTC
TATCCCAGCGACATCGCCGTGGAGTGGGAGAGCAATGGGCAGCCGGA
GAACAACTACAAGACCACGCCTCCCGTGCTGGACTCCGACGGCTCCT
TCTTCCTCTACAGCAAGCTCACCGTGGACAAGAGCAGGTGGCAGCAG
GGGAACGTCTTCTCATGCTCCGTGATGCATGAGGCTCTGCACAACCA
CTACACGCAGAAGTCCCTCTCCCTGTCTCCGGGTAAATGA
HC Amino Acid Sequence
(N297Q is indicated in bold)
(SEQ ID NO: 177)
QVQLVESGGGVVQPGTSLRLSCAASGFTFSSYGMHWVRQAPGKGLEW
VAVIWIDGSNKYYADSVKGRFTISRDNSKNTLYLQMNSLRAEDTAVY
YCARRGGIVVAAPFDYWGQGTLVTVSSASTKGPSVFPLAPSSKSTSG
GTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWY
VDGVEVHNAKTKPREEQYQSTYRVVSVLTVLHQDWLNGKEYKCKVSN
KALPAPIEKTISKAKGQPREPQVYTLPPSRDELTKNQVSLTCLVKGF
YPSDIAVEWESNGQPENNYKTTPPVLDSDGSFFLYSKLTVDKSRWQQ
GNVFSCSVMHEALHNHYTQKSLSLSPGK
LC DNA Sequence
(SEQ ID NO: 350)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 178)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC
REGN14647
HCVR DNA Sequence
(SEQ ID NO: 351)
CAGGTGCAGCTGGTGGAGTCTGGGGGAGGCGTGGTCCAGCCTGGGAG
GTCCCTGAGACTCTCCTGTACAGCGTCTGGAATCACCTTCAGAAATT
ATGGCATGCACTGGGTCCGCCAGGCTCCAGGCAAGGGGCTGGAGTGG
GTGGCAGTTATGTGGTATGATGGAAGTAATAAGTACTATGCAGACTC
CGTGAAGGGCCGTTTCACCATCTCCGGAGACAATTCCAAGGTGTATC
TGCAAATGAACAGCCTGAGAGCCGAGGACACGGCTGTATATTACTGT
GCGAGAAGGGGCACTATAAGAACAGCTGCCCCTTTTGACTACTGGGG
TCAGGGAACCCTGGTCACCGTCTCCTCA
HCVR Amino Acid Sequence
(SEQ ID NO: 137)
QVQLVESGGGVVQPGRSLRLSCTASGITFRNYGMHWVRQAPGKGLEW
VAVMWYDGSNKYYADSVKGRFTISGDNSKVYLQMNSLRAEDTAVYYC
ARRGTIRTAAPFDYWGQGTLVTVSS
HCDR1 DNA Sequence
(SEQ ID NO: 352)
GGAATCACCTTCAGAAATTATGGC
HCDR1 Amino Acid Sequence
(SEQ ID NO: 138)
GITFRNYG
HCDR2 DNA Sequence
(SEQ ID NO: 353)
ATGTGGTATGATGGAAGTAATAAG
HCDR2 Amino Acid Sequence
(SEQ ID NO: 139)
MWYDGSNK
HCDR3 DNA Sequence
(SEQ ID NO: 354)
GCGAGAAGGGGCACTATAAGAACAGCTGCCCCTTTTGACTAC
HCDR3 Amino Acid Sequence
(SEQ ID NO: 140)
ARRGTIRTAAPFDY
LCVR DNA Sequence
(SEQ ID NO: 355)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAA
LCVR Amino Acid Sequence
(SEQ ID NO: 141)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIK
LCDR1 DNA Sequence
(SEQ ID NO: 356)
CAGAGCATTAGCAGCTAT
LCDR1 Amino Acid Sequence
(SEQ ID NO: 142)
QSISSY
LCDR2 DNA Sequence
(SEQ ID NO: 357)
GCTGCATCC
LCDR2 Amino Acid Sequence
(SEQ ID NO: 143)
AAS
LCDR3 DNA Sequence
(SEQ ID NO: 358)
CAACAGAGTTACAGTACCCCTCCGATCACC
LCDR3 Amino Acid Sequence
(SEQ ID NO: 144)
QQSYSTPPIT
HC DNA Sequence
(SEQ ID NO: 359)
CAGGTGCAGCTGGTGGAGTCTGGGGGAGGCGTGGTCCAGCCTGGGAG
GTCCCTGAGACTCTCCTGTACAGCGTCTGGAATCACCTTCAGAAATT
ATGGCATGCACTGGGTCCGCCAGGCTCCAGGCAAGGGGCTGGAGTGG
GTGGCAGTTATGTGGTATGATGGAAGTAATAAGTACTATGCAGACTC
CGTGAAGGGCCGTTTCACCATCTCCGGAGACAATTCCAAGGTGTATC
TGCAAATGAACAGCCTGAGAGCCGAGGACACGGCTGTATATTACTGT
GCGAGAAGGGGCACTATAAGAACAGCTGCCCCTTTTGACTACTGGGG
TCAGGGAACCCTGGTCACCGTCTCCTCAGCCTCCACCAAGGGCCCAT
CGGTCTTCCCCCTGGCACCCTCCTCCAAGAGCACCTCTGGGGGCACA
GCGGCCCTGGGCTGCCTGGTCAAGGACTACTTCCCCGAACCGGTGAC
GGTGTCGTGGAACTCAGGCGCCCTGACCAGCGGCGTGCACACCTTCC
CGGCTGTCCTACAGTCCTCAGGACTCTACTCCCTCAGCAGCGTGGTG
ACCGTGCCCTCCAGCAGCTTGGGCACCCAGACCTACATCTGCAACGT
GAATCACAAGCCCAGCAACACCAAGGTGGACAAGAAAGTTGAGCCCA
AATCTTGTGACAAAACTCACACATGCCCACCGTGCCCAGCACCTGAA
CTCCTGGGGGGACCGTCAGTCTTCCTCTTCCCCCCAAAACCCAAGGA
CACCCTCATGATCTCCCGGACCCCTGAGGTCACATGCGTGGTGGTGG
ACGTGAGCCACGAAGACCCTGAGGTCAAGTTCAACTGGTACGTGGAC
GGCGTGGAGGTGCATAATGCCAAGACAAAGCCGCGGGAGGAGCAGTA
CCAAAGCACGTACCGTGTGGTCAGCGTCCTCACCGTCCTGCACCAGG
ACTGGCTGAATGGCAAGGAGTACAAGTGCAAGGTCTCCAACAAAGCC
CTCCCAGCCCCCATCGAGAAAACCATCTCCAAAGCCAAAGGGCAGCC
CCGAGAACCACAGGTGTACACCCTGCCCCCATCCCGGGATGAGCTGA
CCAAGAACCAGGTCAGCCTGACCTGCCTGGTCAAAGGCTTCTATCCC
AGCGACATCGCCGTGGAGTGGGAGAGCAATGGGCAGCCGGAGAACAA
CTACAAGACCACGCCTCCCGTGCTGGACTCCGACGGCTCCTTCTTCC
TCTACAGCAAGCTCACCGTGGACAAGAGCAGGTGGCAGCAGGGGAAC
GTCTTCTCATGCTCCGTGATGCATGAGGCTCTGCACAACCACTACAC
GCAGAAGTCCCTCTCCCTGTCTCCGGGTAAATGA
HC Amino Acid Sequence
(SEQ ID NO: 179)
QVQLVESGGGVVQPGRSLRLSCTASGITFRNYGMHWVRQAPGKGLEW
VAVMWYDGSNKYYADSVKGRFTISGDNSKVYLQMNSLRAEDTAVYYC
ARRGTIRTAAPFDYWGQGTLVTVSSASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYQSTYRVVSVLTVLHQDWLNGKEYKCKVSNKA
LPAPIEKTISKAKGQPREPQVYTLPPSRDELTKNQVSLTCLVKGFYP
SDIAVEWESNGQPENNYKTTPPVLDSDGSFFLYSKLTVDKSRWQQGN
VFSCSVMHEALHNHYTQKSLSLSPGK
LC DNA Sequence
(SEQ ID NO: 360)
GACATCCAGATGACCCAGTCTCCATCCTCCCTGTCTGCATCTGTAGG
AGACAGAGTCACCATCACTTGCCGGGCAAGTCAGAGCATTAGCAGCT
ATTTAAATTGGTATCAGCAGAAACCAGGGAAAGCCCCTAAGCTCCTG
ATCTATGCTGCATCCAGTTTGCAAAGTGGGGTCCCGTCAAGGTTCAG
TGGCAGTGGATCTGGGACAGATTTCACTCTCACCATCAGCAGTCTGC
AACCTGAAGATTTTGCAACTTACTACTGTCAACAGAGTTACAGTACC
CCTCCGATCACCTTCGGCCAAGGGACACGACTGGAGATTAAACGAAC
TGTGGCTGCACCATCTGTCTTCATCTTCCCGCCATCTGATGAGCAGT
TGAAATCTGGAACTGCCTCTGTTGTGTGCCTGCTGAATAACTTCTAT
CCCAGAGAGGCCAAAGTACAGTGGAAGGTGGATAACGCCCTCCAATC
GGGTAACTCCCAGGAGAGTGTCACAGAGCAGGACAGCAAGGACAGCA
CCTACAGCCTCAGCAGCACCCTGACGCTGAGCAAAGCAGACTACGAG
AAACACAAAGTCTACGCCTGCGAAGTCACCCATCAGGGCCTGAGCTC
GCCCGTCACAAAGAGCTTCAACAGGGGAGAGTGTTAG
LC Amino Acid Sequence
(SEQ ID NO: 180)
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PPITFGQGTRLEIKRTVAAPSVFIFPPSDEQLKSGTASVVCLLNNFY
PREAKVQWKVDNALQSGNSQESVTEQDSKDSTYSLSSTLTLSKADYE
KHKVYACEVTHQGLSSPVTKSFNRGEC
REGN10712
HCVR DNA Sequence
(SEQ ID NO: 428)
CAGGTGCAGCTGGTGGAGTCTGGGGGAGGCGTGGTCCAGCCTGGGAG
GTCCCTGAGACTCTCCTGTGCAGCCTCTGGATTCACATTCAGTACCT
ATGGCATGTACTGGGTCCGCCAGACTCCAGGCAAGGGGCTGGAGTGG
GTGACAGTTATATCATTTGATGGAAATAAAAAATACTATGCAGACTC
CGTGAAGGGCCGATTCACCATCTCCAGAGACAATTCCAAAAACACGC
TGTTTCTGCAAATGAACAGCCTGAAAACTGAGGACACGGCTGTATAT
TACTGTGCGAAATCTTCTAACTGGAACTACGGTTCTTTTGATATATG
GGGCCAAGGGACAATGGTCACCGTCTCTTCA
HCVR Amino Acid Sequence
(SEQ ID NO: 429)
QVQLVESGGGVVQPGRSLRLSCAASGFTFSTYGMYWVRQTPGKGLEW
VTVISFDGNKKYYADSVKGRFTISRDNSKNTLFLQMNSLKTEDTAVY
YCAKSSNWNYGSFDIWGQGTMVTVSS
HCDR1 DNA Sequence
(SEQ ID NO: 430)
GGATTCACATTCAGTACCTATGGC
HCDR1 Amino Acid Sequence
(SEQ ID NO: 431)
GFTFSTYG
HCDR2 DNA Sequence
(SEQ ID NO: 432)
ATATCATTTGATGGAAATAAAAAA
HCDR2 Amino Acid Sequence
(SEQ ID NO: 433)
ISFDGNKK
HCDR3 DNA Sequence
(SEQ ID NO: 434)
GCGAAATCTTCTAACTGGAACTACGGTTCTTTTGATATA
HCDR3 Amino Acid Sequence
(SEQ ID NO: 435)
AKSSNWNYGSFDI
LCVR DNA Sequence
(SEQ ID NO: 436)
CAGTCTGTGCTGACGCAGCCGCCCTCAGTGTCTGGGGCCCCAGGGCA
GAGGGTCACCATCTCCTGCACTGGGAGCAGCTCCAACATCGGGGCAG
GTTATGATGTACACTGGTACCAGCAGCTTCCAGGCACAGCCCCCAGA
CTCCTCATCTCTTATAACAGCAATCGGCCCTCAGGGGTCCCTGACCG
ATTCTCTGGCTCCAAGTCTGGCACCTCAGCCTCCCTGGCCATCACTG
GGCTCCAGGCTGAGGATGAGGCTGACTATTACTGCCAGTCCTATGAC
AGAAGCCTGAGTGGTTCTGTGTTCGGAGGAGGCACCCAGCTGACCGT
CCTC
LCVR Amino Acid Sequence
(SEQ ID NO: 437)
QSVLTQPPSVSGAPGQRVTISCTGSSSNIGAGYDVHWYQQLPGTAPR
LLISYNSNRPSGVPDRFSGSKSGTSASLAITGLQAEDEADYYCQSYD
RSLSGSVFGGGTQLTVL
LCDR1 DNA Sequence
(SEQ ID NO: 438)
AGCTCCAACATCGGGGCAGGTTATGAT
LCDR1 Amino Acid Sequence
(SEQ ID NO: 439)
SSNIGAGYD
LCDR2 DNA Sequence
TATAACAGC
LCDR2 Amino Acid Sequence
YNS
LCDR3 DNA Sequence
(SEQ ID NO: 440)
CAGTCCTATGACAGAAGCCTGAGTGGTTCTGTG
LCDR3 Amino Acid Sequence
(SEQ ID NO: 441)
QSYDRSLSGSV
HC DNA Sequence
(SEQ ID NO: 442)
CAGGTGCAGCTGGTGGAGTCTGGGGGAGGCGTGGTCCAGCCTGGGAG
GTCCCTGAGACTCTCCTGTGCAGCCTCTGGATTCACATTCAGTACCT
ATGGCATGTACTGGGTCCGCCAGACTCCAGGCAAGGGGCTGGAGTGG
GTGACAGTTATATCATTTGATGGAAATAAAAAATACTATGCAGACTC
CGTGAAGGGCCGATTCACCATCTCCAGAGACAATTCCAAAAACACGC
TGTTTCTGCAAATGAACAGCCTGAAAACTGAGGACACGGCTGTATAT
TACTGTGCGAAATCTTCTAACTGGAACTACGGTTCTTTTGATATATG
GGGCCAAGGGACAATGGTCACCGTCTCTTCAGCCTCCACCAAGGGCC
CATCGGTCTTCCCCCTGGCGCCCTGCTCCAGGAGCACCTCCGAGAGC
ACAGCCGCCCTGGGCTGCCTGGTCAAGGACTACTTCCCCGAACCGGT
GACGGTGTCGTGGAACTCAGGCGCCCTGACCAGCGGCGTGCACACCT
TCCCGGCTGTCCTACAGTCCTCAGGACTCTACTCCCTCAGCAGCGTG
GTGACCGTGCCCTCCAGCAGCTTGGGCACGAAGACCTACACCTGCAA
CGTAGATCACAAGCCCAGCAACACCAAGGTGGACAAGAGAGTTGAGT
CCAAATATGGTCCCCCATGCCCACCCTGCCCAGCACCTGAGTTCCTG
GGGGGACCATCAGTCTTCCTGTTCCCCCCAAAACCCAAGGACACTCT
CATGATCTCCCGGACCCCTGAGGTCACGTGCGTGGTGGTGGACGTGA
GCCAGGAAGACCCCGAGGTCCAGTTCAACTGGTACGTGGATGGCGTG
GAGGTGCATAATGCCAAGACAAAGCCGCGGGAGGAGCAGTTCAACAG
CACGTACCGTGTGGTCAGCGTCCTCACCGTCCTGCACCAGGACTGGC
TGAACGGCAAGGAGTACAAGTGCAAGGTCTCCAACAAAGGCCTCCCG
TCCTCCATCGAGAAAACCATCTCCAAAGCCAAAGGGCAGCCCCGAGA
GCCACAGGTGTACACCCTGCCCCCATCCCAGGAGGAGATGACCAAGA
ACCAGGTCAGCCTGACCTGCCTGGTCAAAGGCTTCTACCCCAGCGAC
ATCGCCGTGGAGTGGGAGAGCAATGGGCAGCCGGAGAACAACTACAA
GACCACGCCTCCCGTGCTGGACTCCGACGGCTCCTTCTTCCTCTACA
GCAGGCTCACCGTGGACAAGAGCAGGTGGCAGGAGGGGAATGTCTTC
TCATGCTCCGTGATGCATGAGGCTCTGCACAACCACTACACACAGAA
GTCCCTCTCCCTGTCTCTGGGTAAATGA
HC Amino Acid Sequence
(SEQ ID NO: 443)
QVQLVESGGGVVQPGRSLRLSCAASGFTFSTYGMYWVRQTPGKGLEW
VTVISFDGNKKYYADSVKGRFTISRDNSKNTLFLQMNSLKTEDTAVY
YCAKSSNWNYGSFDIWGQGTMVTVSSASTKGPSVFPLAPCSRSTSES
TAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSD
IAVEWESNGQPENNYKTTPPVLDSDGSFFLYSRLTVDKSRWQEGNVF
SCSVMHEALHNHYTQKSLSLSLGK
HC constant, hIgG4 (S108P) DNA Sequence
(SEQ ID NO: 444)
GCCTCCACCAAGGGCCCATCGGTCTTCCCCCTGGCGCCCTGCTCCAG
GAGCACCTCCGAGAGCACAGCCGCCCTGGGCTGCCTGGTCAAGGACT
ACTTCCCCGAACCGGTGACGGTGTCGTGGAACTCAGGCGCCCTGACC
AGCGGCGTGCACACCTTCCCGGCTGTCCTACAGTCCTCAGGACTCTA
CTCCCTCAGCAGCGTGGTGACCGTGCCCTCCAGCAGCTTGGGCACGA
AGACCTACACCTGCAACGTAGATCACAAGCCCAGCAACACCAAGGTG
GACAAGAGAGTTGAGTCCAAATATGGTCCCCCATGCCCACCCTGCCC
AGCACCTGAGTTCCTGGGGGGACCATCAGTCTTCCTGTTCCCCCCAA
AACCCAAGGACACTCTCATGATCTCCCGGACCCCTGAGGTCACGTGC
GTGGTGGTGGACGTGAGCCAGGAAGACCCCGAGGTCCAGTTCAACTG
GTACGTGGATGGCGTGGAGGTGCATAATGCCAAGACAAAGCCGCGGG
AGGAGCAGTTCAACAGCACGTACCGTGTGGTCAGCGTCCTCACCGTC
CTGCACCAGGACTGGCTGAACGGCAAGGAGTACAAGTGCAAGGTCTC
CAACAAAGGCCTCCCGTCCTCCATCGAGAAAACCATCTCCAAAGCCA
AAGGGCAGCCCCGAGAGCCACAGGTGTACACCCTGCCCCCATCCCAG
GAGGAGATGACCAAGAACCAGGTCAGCCTGACCTGCCTGGTCAAAGG
CTTCTACCCCAGCGACATCGCCGTGGAGTGGGAGAGCAATGGGCAGC
CGGAGAACAACTACAAGACCACGCCTCCCGTGCTGGACTCCGACGGC
TCCTTCTTCCTCTACAGCAGGCTCACCGTGGACAAGAGCAGGTGGCA
GGAGGGGAATGTCTTCTCATGCTCCGTGATGCATGAGGCTCTGCACA
ACCACTACACACAGAAGTCCCTCTCCCTGTCTCTGGGTAAATGA
HC constant, hIgG4 (S108P) Amino Acid Sequence
(SEQ ID NO: 445)
ASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTC
VVVDVSQEDPEVQFNWYVDGVEVHNAKTKPREEQFNSTYRWVSVLTV
LHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
LC DNA Sequence
(SEQ ID NO: 446)
CAGTCTGTGCTGACGCAGCCGCCCTCAGTGTCTGGGGCCCCAGGGCA
GAGGGTCACCATCTCCTGCACTGGGAGCAGCTCCAACATCGGGGCAG
GTTATGATGTACACTGGTACCAGCAGCTTCCAGGCACAGCCCCCAGA
CTCCTCATCTCTTATAACAGCAATCGGCCCTCAGGGGTCCCTGACCG
ATTCTCTGGCTCCAAGTCTGGCACCTCAGCCTCCCTGGCCATCACTG
GGCTCCAGGCTGAGGATGAGGCTGACTATTACTGCCAGTCCTATGAC
AGAAGCCTGAGTGGTTCTGTGTTCGGAGGAGGCACCCAGCTGACCGT
CCTCGGCCAGCCCAAGGCCGCCCCCTCCGTGACCCTGTTCCCCCCCT
CCTCCGAGGAGCTGCAGGCCAACAAGGCCACCCTGGTGTGCCTGATC
TCCGACTTCTACCCCGGCGCCGTGACCGTGGCCTGGAAGGCCGACTC
CTCCCCCGTGAAGGCCGGCGTGGAGACCACCACCCCCTCCAAGCAGT
CCAACAACAAGTACGCCGCCTCCTCCTACCTGTCCCTGACCCCCGAG
CAGTGGAAGTCCCACCGGTCCTACTCCTGCCAGGTGACCCACGAGGG
CTCCACCGTGGAGAAGACCGTGGCCCCCACCGAGTGCTCCTGA
LC Amino Acid Sequence
(SEQ ID NO: 447)
QSVLTQPPSVSGAPGQRVTISCTGSSSNIGAGYDVHWYQQLPGTAPR
LLISYNSNRPSGVPDRFSGSKSGTSASLAITGLQAEDEADYYCQSYD
RSLSGSVFGGGTQLTVLGQPKAAPSVTLFPPSSEELQANKATLVCLI
SDFYPGAVTVAWKADSSPVKAGVETTTPSKQSNNKYAASSYLSLTPE
QWKSHRSYSCQVTHEGSTVEKTVAPTECS
LC constant, hLamda DNA Sequence
(SEQ ID NO: 448)
GGCCAGCCCAAGGCCGCCCCCTCCGTGACCCTGTTCCCCCCCTCCTC
CGAGGAGCTGCAGGCCAACAAGGCCACCCTGGTGTGCCTGATCTCCG
ACTTCTACCCCGGCGCCGTGACCGTGGCCTGGAAGGCCGACTCCTCC
CCCGTGAAGGCCGGCGTGGAGACCACCACCCCCTCCAAGCAGTCCAA
CAACAAGTACGCCGCCTCCTCCTACCTGTCCCTGACCCCCGAGCAGT
GGAAGTCCCACCGGTCCTACTCCTGCCAGGTGACCCACGAGGGCTCC
ACCGTGGAGAAGACCGTGGCCCCCACCGAGTGCTCCTGA
LC constant, hLamda Amino Acid Sequence
(SEQ ID NO: 449)
GQPKAAPSVTLFPPSSEELQANKATLVCLISDFYPGAVTVAWKADSS
PVKAGVETTTPSKQSNNKYAASSYLSLTPEQWKSHRSYSCQVTHEGS
TVEKTVAPTECS
REGN9908
HCVR DNA Sequence
(SEQ ID NO: 450)
GAGGTGCAGTTGTTGGAGTCTGGGGGAGGCTTGGCACAGCCTGGGGG
GTCCCTGAGACTCTCCTGTGCAGCCTCTGGATTCACCTTTAGCAGTT
ATGCCATGAGCTGGGTCCGCCAGGCTCCAGGGAAGGGGCTGGAGTGG
GTCTCATCTGTTAGTGGTAGTGGTGGTACCACATATTATGCAGCCTC
CGTGAAGGGCCGGTTCACCGTCTCCAGAGACAATTCCAAGAAGACGC
TCTATCTGCAAATGAACAGCCTGAGAGCCGAGGACACGGCCGTATAT
TACTGTGGGAAAGGAGGATATTGTAGTAGTAGTGGTTGCCGTCACTA
CGGTATGGACGTCTGGGGCCAAGGGACCACGGTCACCGTCTCCGCA
HCVR Amino Acid Sequence
(SEQ ID NO: 451)
EVQLLESGGGLAQPGGSLRLSCAASGFTFSSYAMSWVRQAPGKGLEW
VSSVSGSGGTTYYAASVKGRFTVSRDNSKKTLYLQMNSLRAEDTAVY
YCGKGGYCSSSGCRHYGMDVWGQGTTVTVSA
HCDR1 DNA Sequence
(SEQ ID NO: 452)
GGATTCACCTTTAGCAGTTATGCC
HCDR1 Amino Acid Sequence
(SEQ ID NO: 453)
GFTFSSYA
HCDR2 DNA Sequence
(SEQ ID NO: 454)
GTTAGTGGTAGTGGTGGTACCACA
HCDR2 Amino Acid Sequence
(SEQ ID NO: 455)
VSGSGGTT
HCDR3 DNA Sequence
(SEQ ID NO: 456)
GGGAAAGGAGGATATTGTAGTAGTAGTGGTTGCCGTCACTACGGTAT
GGACGTC
HCDR3 Amino Acid Sequence
(SEQ ID NO: 457)
GKGGYCSSSGCRHYGMDV
LCVR DNA Sequence
(SEQ ID NO: 458)
CAGTCTGTGCTGACTCAGCCACCCTCAGCGTCTGGGACCCCCGGGCA
GAGGGTCGCCATTTCTTGTTCTGGAAGCAACTCCAACATCGGAAATA
ATTACTTATACTGGTACCAGCAGATCCCAGGAACGACCCCCAAACTC
CTCATCTATAGAAATAATCAGCGGCCCTCAGGGGTCCCTGACCGATT
CTCTGCCTCCAAGTCTGGCACCTCAGCCTCCCTGGCCATCAGTGGGC
TCCGGTCCGGGGATGAGGCTGATTATTACTGTGCAGCATGGGATGAC
AGCCTGAGTGGGTATGTCTTCGGAACTGGGACCAAGGTCACCGTCCT
A
LCVR Amino Acid Sequence
(SEQ ID NO: 459)
QSVLTQPPSASGTPGQRVAISCSGSNSNIGNNYLYWYQQIPGTTPKL
LIYRNNQRPSGVPDRFSASKSGTSASLAISGLRSGDEADYYCAAWDD
SLSGYVFGTGTKVTVL
LCDR1 DNA Sequence
(SEQ ID NO: 460)
AACTCCAACATCGGAAATAATTA
LCDR1 Amino Acid Sequence
(SEQ ID NO: 461)
NSNIGNN
LCDR2 DNA Sequence
AGAAATAAT
LCDR2 Amino Acid Sequence
RNN
LCDR3 DNA Sequence
GCAGCATGGGATGACAGCCTGAGTGGGTATGTC
(SEQ ID NO: 462)
LCDR3 Amino Acid Sequence
AAWDDSLSGYV
(SEQ ID NO: 463)
HC DNA Sequence
(SEQ ID NO: 464)
GAGGTGCAGTTGTTGGAGTCTGGGGGAGGCTTGGCACAGCCTGGGGG
GTCCCTGAGACTCTCCTGTGCAGCCTCTGGATTCACCTTTAGCAGTT
ATGCCATGAGCTGGGTCCGCCAGGCTCCAGGGAAGGGGCTGGAGTGG
GTCTCATCTGTTAGTGGTAGTGGTGGTACCACATATTATGCAGCCTC
CGTGAAGGGCCGGTTCACCGTCTCCAGAGACAATTCCAAGAAGACGC
TCTATCTGCAAATGAACAGCCTGAGAGCCGAGGACACGGCCGTATAT
TACTGTGGGAAAGGAGGATATTGTAGTAGTAGTGGTTGCCGTCACTA
CGGTATGGACGTCTGGGGCCAAGGGACCACGGTCACCGTCTCCGCAG
CCAAGACAACACCTCCTTCTGTGTATCCTCTGGCTCCTGGATGTGGA
GATACAACAGGATCTTCTGTGACACTGGGATGTCTGGTGAAGGGATA
TTTTCCTGAATCTGTGACAGTGACATGGAACTCTGGATCTCTGTCTT
CTTCTGTGCATACATTTCCTGCTCTGCTGCAGTCTGGACTGTATACA
ATGTCTTCTTCTGTGACAGTGCCTTCTTCTACATGGCCTTCTCAGAC
AGTGACATGTTCTGTGGCTCATCCTGCTTCTTCTACAACAGTGGATA
AGAAGCTGGAACCTTCTGGACCTATCTCTACAATCAATCCTTGTCCT
CCTTGTAAGGAATGTCATAAGTGTCCTGCTCCTAATCTGGAAGGAGG
ACCTTCTGTGTTTATCTTTCCTCCTAATATCAAGGATGTGCTGATGA
TCTCTCTGACACCTAAGGTGACATGTGTGGTGGTGGATGTGTCTGAA
GATGATCCTGATGTGCAGATCTCTTGGTTTGTGAATAATGTGGAAGT
GCATACAGCTCAGACACAGACACATAGAGAAGATTATAATTCTACAA
TCAGAGTGGTGTCTACACTGCCTATCCAGCATCAGGATTGGATGTCT
GGAAAGGAATTTAAGTGTAAGGTGAATAATAAGGATCTGCCTTCTCC
TATCGAAAGAACAATCTCTAAGATCAAGGGACTGGTGAGAGCTCCTC
AGGTGTATATCCTGCCTCCTCCTGCTGAACAGCTGTCCAGAAAGGAT
GTGTCTCTGACATGTCTGGTGGTGGGATTTAATCCTGGAGATATCTC
TGTGGAATGGACATCTAATGGACATACAGAAGAAAATTATAAGGATA
CAGCTCCTGTGCTGGATTCTGATGGATCTTATTTTATCTATTCTAAG
CTGAATATGAAGACATCTAAGTGGGAAAAGACAGATTCTTTTTCTTG
TAATGTGAGACATGAAGGACTGAAGAATTATTATCTGAAGAAGACAA
TCTCCAGATCTCCTGGAAAGTGA
HC Amino Acid Sequence
(SEQ ID NO: 465)
EVQLLESGGGLAQPGGSLRLSCAASGFTFSSYAMSWVRQAPGKGLEW
VSSVSGSGGTTYYAASVKGRFTVSRDNSKKTLYLQMNSLRAEDTAVY
YCGKGGYCSSSGCRHYGMDVWGQGTTVTVSAAKTTPPSVYPLAPGCG
DTTGSSVTLGCLVKGYFPESVTVTWNSGSLSSSVHTFPALLQSGLYT
MSSSVTVPSSTWPSQTVTCSVAHPASSTTVDKKLEPSGPISTINPCP
PCKECHKCPAPNLEGGPSVFIFPPNIKDVLMISLTPKVTCVVVDVSE
DDPDVQISWFVNNVEVHTAQTQTHREDYNSTIRVVSTLPIQHQDWMS
GKEFKCKVNNKDLPSPIERTISKIKGLVRAPQVYILPPPAEQLSRKD
VSLTCLVVGFNPGDISVEWTSNGHTEENYKDTAPVLDSDGSYFIYSK
LNMKTSKWEKTDSFSCNVRHEGLKNYYLKKTISRSPGK
HC constant, mIgG2b DNA Sequence
(SEQ ID NO: 466)
GCCAAGACAACACCTCCTTCTGTGTATCCTCTGGCTCCTGGATGTGG
AGATACAACAGGATCTTCTGTGACACTGGGATGTCTGGTGAAGGGAT
ATTTTCCTGAATCTGTGACAGTGACATGGAACTCTGGATCTCTGTCT
TCTTCTGTGCATACATTTCCTGCTCTGCTGCAGTCTGGACTGTATAC
AATGTCTTCTTCTGTGACAGTGCCTTCTTCTACATGGCCTTCTCAGA
CAGTGACATGTTCTGTGGCTCATCCTGCTTCTTCTACAACAGTGGAT
AAGAAGCTGGAACCTTCTGGACCTATCTCTACAATCAATCCTTGTCC
TCCTTGTAAGGAATGTCATAAGTGTCCTGCTCCTAATCTGGAAGGAG
GACCTTCTGTGTTTATCTTTCCTCCTAATATCAAGGATGTGCTGATG
ATCTCTCTGACACCTAAGGTGACATGTGTGGTGGTGGATGTGTCTGA
AGATGATCCTGATGTGCAGATCTCTTGGTTTGTGAATAATGTGGAAG
TGCATACAGCTCAGACACAGACACATAGAGAAGATTATAATTCTACA
ATCAGAGTGGTGTCTACACTGCCTATCCAGCATCAGGATTGGATGTC
TGGAAAGGAATTTAAGTGTAAGGTGAATAATAAGGATCTGCCTTCTC
CTATCGAAAGAACAATCTCTAAGATCAAGGGACTGGTGAGAGCTCCT
CAGGTGTATATCCTGCCTCCTCCTGCTGAACAGCTGTCCAGAAAGGA
TGTGTCTCTGACATGTCTGGTGGTGGGATTTAATCCTGGAGATATCT
CTGTGGAATGGACATCTAATGGACATACAGAAGAAAATTATAAGGAT
ACAGCTCCTGTGCTGGATTCTGATGGATCTTATTTTATCTATTCTAA
GCTGAATATGAAGACATCTAAGTGGGAAAAGACAGATTCTTTTTCTT
GTAATGTGAGACATGAAGGACTGAAGAATTATTATCTGAAGAAGACA
ATCTCCAGATCTCCTGGAAAGTGA
HC constant, mIgG2b Amino Acid Sequence
(SEQ ID NO: 467)
AKTTPPSVYPLAPGCGDTTGSSVTLGCLVKGYFPESVTVTWNSGSLS
SSVHTFPALLQSGLYTMSSSVTVPSSTWPSQTVTCSVAHPASSTTVD
KKLEPSGPISTINPCPPCKECHKCPAPNLEGGPSVFIFPPNIKDVLM
ISLTPKVTCVVVDVSEDDPDVQISWFVNNVEVHTAQTQTHREDYNST
IRVVSTLPIQHQDWMSGKEFKCKVNNKDLPSPIERTISKIKGLVRAP
QVYILPPPAEQLSRKDVSLTCLVVGFNPGDISVEWTSNGHTEENYKD
TAPVLDSDGSYFIYSKLNMKTSKWEKTDSFSCNVRHEGLKNYYLKKT
ISRSPGK
LC DNA Sequence
(SEQ ID NO: 468)
CAGTCTGTGCTGACTCAGCCACCCTCAGCGTCTGGGACCCCCGGGCA
GAGGGTCGCCATTTCTTGTTCTGGAAGCAACTCCAACATCGGAAATA
ATTACTTATACTGGTACCAGCAGATCCCAGGAACGACCCCCAAACTC
CTCATCTATAGAAATAATCAGCGGCCCTCAGGGGTCCCTGACCGATT
CTCTGCCTCCAAGTCTGGCACCTCAGCCTCCCTGGCCATCAGTGGGC
TCCGGTCCGGGGATGAGGCTGATTATTACTGTGCAGCATGGGATGAC
AGCCTGAGTGGGTATGTCTTCGGAACTGGGACCAAGGTCACCGTCCT
AGGCCAACCTAAATCATCTCCATCCGTTACTCTCTTCCCCCCATCTT
CAGAAGAACTCGAAACCAATAAAGCCACACTCGTTTGCACCATTACA
GATTTCTATCCAGGAGTAGTTACAGTCGATTGGAAAGTAGACGGAAC
ACCAGTTACACAGGGTATGGAAACCACACAACCATCTAAGCAGTCTA
ATAACAAATACATGGCCTCATCATACCTCACTCTTACCGCCCGCGCA
TGGGAAAGACATTCATCATATTCTTGCCAGGTAACCCACGAAGGACA
CACAGTTGAAAAATCTTTGAGTAGAGCAGATTGTAGTTGA
LC Amino Acid Sequence
(SEQ ID NO: 469)
QSVLTQPPSASGTPGQRVAISCSGSNSNIGNNYLYWYQQIPGTTPKL
LIYRNNQRPSGVPDRFSASKSGTSASLAISGLRSGDEADYYCAAWDD
SLSGYVFGTGTKVTVLGQPKSSPSVTLFPPSSEELETNKATLVCTIT
DFYPGVVTVDWKVDGTPVTQGMETTQPSKQSNNKYMASSYLTLTARA
WERHSSYSCQVTHEGHTVEKSLSRADCS
LC constant, mLambda DNA Sequence
(SEQ ID NO: 470)
GGCCAACCTAAATCATCTCCATCCGTTACTCTCTTCCCCCCATCTTC
AGAAGAACTCGAAACCAATAAAGCCACACTCGTTTGCACCATTACAG
ATTTCTATCCAGGAGTAGTTACAGTCGATTGGAAAGTAGACGGAACA
CCAGTTACACAGGGTATGGAAACCACACAACCATCTAAGCAGTCTAA
TAACAAATACATGGCCTCATCATACCTCACTCTTACCGCCCGCGCAT
GGGAAAGACATTCATCATATTCTTGCCAGGTAACCCACGAAGGACAC
ACAGTTGAAAAATCTTTGAGTAGAGCAGATTGTAGTTGA
LC constant, mLambda Amino Acid Sequence
(SEQ ID NO: 471)
GQPKSSPSVTLFPPSSEELETNKATLVCTITDFYPGVVTVDWKVDGT
PVTQGMETTQPSKQSNNKYMASSYLTLTARAWERHSSYSCQVTHEGH
TVEKSLSRADCS

In one aspect, the present disclosure provides an antigen-binding protein that binds specifically Calcium Voltage-Gated Channel Auxiliary Subunit Gamma 1 (CACNG1), comprising: (i) an HCVR that comprises the HCDR1, HCDR2, and HCDR3 of an HCVR comprising the amino acid sequence set forth in SEQ ID NO: 1, 9, 17, 25, 33, 41, 49, 57, 65, 73, 81, 89, 97, 105, 113, 121, 129, 137, 429, or 451 (or a variant thereof); and/or (ii) an LCVR that comprises the LCDR1, LCDR2, and LCDR3 of an LCVR comprising the amino acid sequence set forth in SEQ ID NO: 5, 13, 21, 29, 37, 45, 53, 61, 69, 77, 85, 93, 101, 109, 117, 125, 133, 141, 437, or 459 (or a variant thereof).

In some embodiments, an antigen-binding protein described herein comprises: (1) an HCVR comprising the HCDR1, HCDR2, and HCDR3 of an HCVR that comprises the amino acid sequence set forth in SEQ ID NO: 1 (or a variant thereof); and an LCVR comprising the LCDR1, LCDR2, and LCDR3 of an LCVR that comprises the amino acid sequence set forth in SEQ ID NO: 5 (or a variant thereof); (2) an HCVR comprising the HCDR1, HCDR2, and HCDR3 of an HCVR that comprises the amino acid sequence set forth in SEQ ID NO: 9; and an LCVR comprising the LCDR1, LCDR2, and LCDR3 of an LCVR that comprises the amino acid sequence set forth in SEQ ID NO: 13 (or a variant thereof); (3) an HCVR comprising the HCDR1, HCDR2, and HCDR3 of an HCVR that comprises the amino acid sequence set forth in SEQ ID NO: 17; and an LCVR comprising the LCDR1, LCDR2, and LCDR3 of an LCVR that comprises the amino acid sequence set forth in SEQ ID NO: 21 (or a variant thereof); (4) an HCVR comprising the HCDR1, HCDR2, and HCDR3 of an HCVR that comprises the amino acid sequence set forth in SEQ ID NO: 25 (or a variant thereof); and an LCVR comprising the LCDR1, LCDR2, and LCDR3 of an LCVR that comprises the amino acid sequence set forth in SEQ ID NO: 29 (or a variant thereof); (5) an HCVR comprising the HCDR1, HCDR2, and HCDR3 of an HCVR that comprises the amino acid sequence set forth in SEQ ID NO: 33 (or a variant thereof); and an LCVR comprising the LCDR1, LCDR2, and LCDR3 of an LCVR that comprises the amino acid sequence set forth in SEQ ID NO: 37 (or a variant thereof); (6) an HCVR comprising the HCDR1, HCDR2, and HCDR3 of an HCVR that comprises the amino acid sequence set forth in SEQ ID NO: 41 (or a variant thereof); and an LCVR comprising the LCDR1, LCDR2, and LCDR3 of an LCVR that comprises the amino acid sequence set forth in SEQ ID NO: 45 (or a variant thereof); (7) an HCVR comprising the HCDR1, HCDR2, and HCDR3 of an HCVR that comprises the amino acid sequence set forth in SEQ ID NO: 49 (or a variant thereof); and an LCVR comprising the LCDR1, LCDR2, and LCDR3 of an LCVR that comprises the amino acid sequence set forth in SEQ ID NO: 53 (or a variant thereof); (8) an HCVR comprising the HCDR1, HCDR2, and HCDR3 of an HCVR that comprises the amino acid sequence set forth in SEQ ID NO: 57 (or a variant thereof); and an LCVR comprising the LCDR1, LCDR2, and LCDR3 of an LCVR that comprises the amino acid sequence set forth in SEQ ID NO: 61 (or a variant thereof); (9) an HCVR comprising the HCDR1, HCDR2, and HCDR3 of an HCVR that comprises the amino acid sequence set forth in SEQ ID NO: 65 (or a variant thereof); and an LCVR comprising the LCDR1, LCDR2, and LCDR3 of an LCVR that comprises the amino acid sequence set forth in SEQ ID NO: 69 (or a variant thereof); (10) an HCVR comprising the HCDR1, HCDR2, and HCDR3 of an HCVR that comprises the amino acid sequence set forth in SEQ ID NO: 73 (or a variant thereof); and an LCVR comprising the LCDR1, LCDR2, and LCDR3 of an LCVR that comprises the amino acid sequence set forth in SEQ ID NO: 77 (or a variant thereof); (11) an HCVR comprising the HCDR1, HCDR2, and HCDR3 of an HCVR that comprises the amino acid sequence set forth in SEQ ID NO: 81 (or a variant thereof); and an LCVR comprising the LCDR1, LCDR2, and LCDR3 of an LCVR that comprises the amino acid sequence set forth in SEQ ID NO: 85 (or a variant thereof); (12) an HCVR comprising the HCDR1, HCDR2, and HCDR3 of an HCVR that comprises the amino acid sequence set forth in SEQ ID NO: 89 (or a variant thereof); and an LCVR comprising the LCDR1, LCDR2, and LCDR3 of an LCVR that comprises the amino acid sequence set forth in SEQ ID NO: 93 (or a variant thereof); (13) an HCVR comprising the HCDR1, HCDR2, and HCDR3 of an HCVR that comprises the amino acid sequence set forth in SEQ ID NO: 97 (or a variant thereof); and an LCVR comprising the LCDR1, LCDR2, and LCDR3 of an LCVR that comprises the amino acid sequence set forth in SEQ ID NO: 101 (or a variant thereof); (14) an HCVR comprising the HCDR1, HCDR2, and HCDR3 of an HCVR that comprises the amino acid sequence set forth in SEQ ID NO: 105 (or a variant thereof); and an LCVR comprising the LCDR1, LCDR2, and LCDR3 of an LCVR that comprises the amino acid sequence set forth in SEQ ID NO: 109 (or a variant thereof); (15) an HCVR comprising the HCDR1, HCDR2, and HCDR3 of an HCVR that comprises the amino acid sequence set forth in SEQ ID NO: 113 (or a variant thereof); and an LCVR comprising the LCDR1, LCDR2, and LCDR3 of an LCVR that comprises the amino acid sequence set forth in SEQ ID NO: 117 (or a variant thereof); (16) an HCVR comprising the HCDR1, HCDR2, and HCDR3 of an HCVR that comprises the amino acid sequence set forth in SEQ ID NO: 121 (or a variant thereof); and an LCVR comprising the LCDR1, LCDR2, and LCDR3 of an LCVR that comprises the amino acid sequence set forth in SEQ ID NO: 125 (or a variant thereof); (17) an HCVR comprising the HCDR1, HCDR2, and HCDR3 of an HCVR that comprises the amino acid sequence set forth in SEQ ID NO: 129 (or a variant thereof); and an LCVR comprising the LCDR1, LCDR2, and LCDR3 of an LCVR that comprises the amino acid sequence set forth in SEQ ID NO: 133 (or a variant thereof); (18) an HCVR comprising the HCDR1, HCDR2, and HCDR3 of an HCVR that comprises the amino acid sequence set forth in SEQ ID NO: 137 (or a variant thereof); and an LCVR comprising the LCDR1, LCDR2, and LCDR3 of an LCVR that comprises the amino acid sequence set forth in SEQ ID NO: 141 (or a variant thereof) (19) an HCVR comprising the HCDR1, HCDR2, and HCDR3 of an HCVR that comprises the amino acid sequence set forth in SEQ ID NO: 429 (or a variant thereof); and an LCVR comprising the LCDR1, LCDR2, and LCDR3 of an LCVR that comprises the amino acid sequence set forth in SEQ ID NO: 437 (or a variant thereof); or (20) an HCVR comprising the HCDR1, HCDR2, and HCDR3 of an HCVR that comprises the amino acid sequence set forth in SEQ ID NO: 451 (or a variant thereof); and an LCVR comprising the LCDR1, LCDR2, and LCDR3 of an LCVR that comprises the amino acid sequence set forth in SEQ ID NO: 459 (or a variant thereof).

In some embodiments, an antigen-binding protein described herein comprises: (a) an HCVR that comprises: an HCDR1 comprising the amino acid sequence set forth in SEQ ID NO: 2 (or a variant thereof), an HCDR2 comprising the amino acid sequence set forth in SEQ ID NO: 3 (or a variant thereof), and an HCDR3 comprising the amino acid sequence set forth in SEQ ID NO: 4 (or a variant thereof); and an LCVR that comprises: an LCDR1 comprising the amino acid sequence set forth in SEQ ID NO: 6 (or a variant thereof), an LCDR2 comprising the amino acid sequence set forth in SEQ ID NO: 7 (or a variant thereof), and an LCDR3 comprising the amino acid sequence set forth in SEQ ID NO: 8 (or a variant thereof); (b) an HCVR that comprises: an HCDR1 comprising the amino acid sequence set forth in SEQ ID NO: 10 (or a variant thereof), an HCDR2 comprising the amino acid sequence set forth in SEQ ID NO: 11 (or a variant thereof), and an HCDR3 comprising the amino acid sequence set forth in SEQ ID NO: 12 (or a variant thereof); and an LCVR that comprises: an LCDR1 comprising the amino acid sequence set forth in SEQ ID NO: 14 (or a variant thereof), an LCDR2 comprising the amino acid sequence set forth in SEQ ID NO: 15 (or a variant thereof), and an LCDR3 comprising the amino acid sequence set forth in SEQ ID NO: 16 (or a variant thereof); (c) an HCVR that comprises: an HCDR1 comprising the amino acid sequence set forth in SEQ ID NO: 18 (or a variant thereof), an HCDR2 comprising the amino acid sequence set forth in SEQ ID NO: 19 (or a variant thereof), and an HCDR3 comprising the amino acid sequence set forth in SEQ ID NO: 20 (or a variant thereof); and an LCVR that comprises: an LCDR1 comprising the amino acid sequence set forth in SEQ ID NO: 22 (or a variant thereof), an LCDR2 comprising the amino acid sequence set forth in SEQ ID NO: 23 (or a variant thereof), and an LCDR3 comprising the amino acid sequence set forth in SEQ ID NO: 24 (or a variant thereof); (d) an HCVR that comprises: an HCDR1 comprising the amino acid sequence set forth in SEQ ID NO: 26 (or a variant thereof), an HCDR2 comprising the amino acid sequence set forth in SEQ ID NO: 27 (or a variant thereof), and an HCDR3 comprising the amino acid sequence set forth in SEQ ID NO: 28 (or a variant thereof); and an LCVR that comprises: an LCDR1 comprising the amino acid sequence set forth in SEQ ID NO: 30 (or a variant thereof), an LCDR2 comprising the amino acid sequence set forth in SEQ ID NO: 31 (or a variant thereof), and an LCDR3 comprising the amino acid sequence set forth in SEQ ID NO: 32 (or a variant thereof); (e) an HCVR that comprises: an HCDR1 comprising the amino acid sequence set forth in SEQ ID NO: 34 (or a variant thereof), an HCDR2 comprising the amino acid sequence set forth in SEQ ID NO: 35 (or a variant thereof), and an HCDR3 comprising the amino acid sequence set forth in SEQ ID NO: 36 (or a variant thereof); and an LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof); (f) an HCVR that comprises: an HCDR1 comprising the amino acid sequence set forth in SEQ ID NO: 42 (or a variant thereof), an HCDR2 comprising the amino acid sequence set forth in SEQ ID NO: 43 (or a variant thereof), and an HCDR3 comprising the amino acid sequence set forth in SEQ ID NO: 44 (or a variant thereof); and an LCVR that comprises: an LCDR1 comprising the amino acid sequence set forth in SEQ ID NO: 46 (or a variant thereof), an LCDR2 comprising the amino acid sequence set forth in SEQ ID NO: 47 (or a variant thereof), and an LCDR3 comprising the amino acid sequence set forth in SEQ ID NO: 48 (or a variant thereof); (g) an HCVR that comprises: an HCDR1 comprising the amino acid sequence set forth in SEQ ID NO: 50 (or a variant thereof), an HCDR2 comprising the amino acid sequence set forth in SEQ ID NO: 51 (or a variant thereof), and an HCDR3 comprising the amino acid sequence set forth in SEQ ID NO: 52 (or a variant thereof); and an LCVR that comprises: an LCDR1 comprising the amino acid sequence set forth in SEQ ID NO: 54 (or a variant thereof), an LCDR2 comprising the amino acid sequence set forth in SEQ ID NO: 55 (or a variant thereof), and an LCDR3 comprising the amino acid sequence set forth in SEQ ID NO: 56 (or a variant thereof); (h) an HCVR that comprises: an HCDR1 comprising the amino acid sequence set forth in SEQ ID NO: 58 (or a variant thereof), an HCDR2 comprising the amino acid sequence set forth in SEQ ID NO: 59 (or a variant thereof), and an HCDR3 comprising the amino acid sequence set forth in SEQ ID NO: 60 (or a variant thereof); and an LCVR that comprises: an LCDR1 comprising the amino acid sequence set forth in SEQ ID NO: 62 (or a variant thereof), an LCDR2 comprising the amino acid sequence set forth in SEQ ID NO: 63 (or a variant thereof), and an LCDR3 comprising the amino acid sequence set forth in SEQ ID NO: 64 (or a variant thereof); (i) an HCVR that comprises: an HCDR1 comprising the amino acid sequence set forth in SEQ ID NO: 66 (or a variant thereof), an HCDR2 comprising the amino acid sequence set forth in SEQ ID NO: 67 (or a variant thereof), and an HCDR3 comprising the amino acid sequence set forth in SEQ ID NO: 68 (or a variant thereof); and an LCVR that comprises: an LCDR1 comprising the amino acid sequence set forth in SEQ ID NO: 70 (or a variant thereof), an LCDR2 comprising the amino acid sequence set forth in SEQ ID NO: 71 (or a variant thereof), and an LCDR3 comprising the amino acid sequence set forth in SEQ ID NO: 72 (or a variant thereof); (j) an HCVR that comprises: an HCDR1 comprising the amino acid sequence set forth in SEQ ID NO: 74 (or a variant thereof), an HCDR2 comprising the amino acid sequence set forth in SEQ ID NO: 75 (or a variant thereof), and an HCDR3 comprising the amino acid sequence set forth in SEQ ID NO: 76 (or a variant thereof); and an LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof); (k) an HCVR that comprises: an HCDR1 comprising the amino acid sequence set forth in SEQ ID NO: 82 (or a variant thereof), an HCDR2 comprising the amino acid sequence set forth in SEQ ID NO: 83 (or a variant thereof), and an HCDR3 comprising the amino acid sequence set forth in SEQ ID NO: 84 (or a variant thereof); and an LCVR that comprises: an LCDR1 comprising the amino acid sequence set forth in SEQ ID NO: 86 (or a variant thereof), an LCDR2 comprising the amino acid sequence set forth in SEQ ID NO: 87 (or a variant thereof), and an LCDR3 comprising the amino acid sequence set forth in SEQ ID NO: 88 (or a variant thereof); (l) an HCVR that comprises: an HCDR1 comprising the amino acid sequence set forth in SEQ ID NO: 90 (or a variant thereof), an HCDR2 comprising the amino acid sequence set forth in SEQ ID NO: 91 (or a variant thereof), and an HCDR3 comprising the amino acid sequence set forth in SEQ ID NO: 92 (or a variant thereof); and an LCVR that comprises: an LCDR1 comprising the amino acid sequence set forth in SEQ ID NO: 94 (or a variant thereof), an LCDR2 comprising the amino acid sequence set forth in SEQ ID NO: 95 (or a variant thereof), and an LCDR3 comprising the amino acid sequence set forth in SEQ ID NO: 96 (or a variant thereof); (m) an HCVR that comprises: an HCDR1 comprising the amino acid sequence set forth in SEQ ID NO: 98 (or a variant thereof), an HCDR2 comprising the amino acid sequence set forth in SEQ ID NO: 99 (or a variant thereof), and an HCDR3 comprising the amino acid sequence set forth in SEQ ID NO: 100 (or a variant thereof); and an LCVR that comprises: an LCDR1 comprising the amino acid sequence set forth in SEQ ID NO: 102 (or a variant thereof), an LCDR2 comprising the amino acid sequence set forth in SEQ ID NO: 103 (or a variant thereof), and an LCDR3 comprising the amino acid sequence set forth in SEQ ID NO: 104 (or a variant thereof); (n) an HCVR that comprises: an HCDR1 comprising the amino acid sequence set forth in SEQ ID NO: 106 (or a variant thereof), an HCDR2 comprising the amino acid sequence set forth in SEQ ID NO: 107 (or a variant thereof), and an HCDR3 comprising the amino acid sequence set forth in SEQ ID NO: 108 (or a variant thereof); and an LCVR that comprises: an LCDR1 comprising the amino acid sequence set forth in SEQ ID NO: 110 (or a variant thereof), an LCDR2 comprising the amino acid sequence set forth in SEQ ID NO: 111 (or a variant thereof), and an LCDR3 comprising the amino acid sequence set forth in SEQ ID NO: 112 (or a variant thereof); (o) an HCVR that comprises: an HCDR1 comprising the amino acid sequence set forth in SEQ ID NO: 114 (or a variant thereof), an HCDR2 comprising the amino acid sequence set forth in SEQ ID NO: 115 (or a variant thereof), and an HCDR3 comprising the amino acid sequence set forth in SEQ ID NO: 116 (or a variant thereof); and an LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 102 (or a variant thereof); (p) an HCVR that comprises: an HCDR1 comprising the amino acid sequence set forth in SEQ ID NO: 122 (or a variant thereof), an HCDR2 comprising the amino acid sequence set forth in SEQ ID NO: 123 (or a variant thereof), and an HCDR3 comprising the amino acid sequence set forth in SEQ ID NO: 124 (or a variant thereof); and an LCVR that comprises: an LCDR1 comprising the amino acid sequence set forth in SEQ ID NO: 126 (or a variant thereof), an LCDR2 comprising the amino acid sequence set forth in SEQ ID NO: 127 (or a variant thereof), and an LCDR3 comprising the amino acid sequence set forth in SEQ ID NO: 128 (or a variant thereof); (q) an HCVR that comprises: an HCDR1 comprising the amino acid sequence set forth in SEQ ID NO: 130 (or a variant thereof), an HCDR2 comprising the amino acid sequence set forth in SEQ ID NO: 131 (or a variant thereof), and an HCDR3 comprising the amino acid sequence set forth in SEQ ID NO: 132 (or a variant thereof); and an LCVR that comprises: an LCDR1 comprising the amino acid sequence set forth in SEQ ID NO: 134 (or a variant thereof), an LCDR2 comprising the amino acid sequence set forth in SEQ ID NO: 135 (or a variant thereof), and an LCDR3 comprising the amino acid sequence set forth in SEQ ID NO: 136 (or a variant thereof); (r) an HCVR that comprises: an HCDR1 comprising the amino acid sequence set forth in SEQ ID NO: 138 (or a variant thereof), an HCDR2 comprising the amino acid sequence set forth in SEQ ID NO: 139 (or a variant thereof), and an HCDR3 comprising the amino acid sequence set forth in SEQ ID NO: 140 (or a variant thereof); and an LCVR that comprises: an LCDR1 comprising the amino acid sequence set forth in SEQ ID NO: 142 (or a variant thereof), an LCDR2 comprising the amino acid sequence set forth in SEQ ID NO: 143 (or a variant thereof), and an LCDR3 comprising the amino acid sequence set forth in SEQ ID NO: 144 (or a variant thereof); (s) an HCVR that comprises: an HCDR1 comprising the amino acid sequence set forth in SEQ ID NO: 431 (or a variant thereof), an HCDR2 comprising the amino acid sequence set forth in SEQ ID NO: 433 (or a variant thereof), and an HCDR3 comprising the amino acid sequence set forth in SEQ ID NO: 435 (or a variant thereof); and an LCVR that comprises: an LCDR1 comprising the amino acid sequence set forth in SEQ ID NO: 439 (or a variant thereof), an LCDR2 comprising the amino acid sequence YNS (or a variant thereof), and an LCDR3 comprising the amino acid sequence set forth in SEQ ID NO: 441 (or a variant thereof); or (t) an HCVR that comprises: an HCDR1 comprising the amino acid sequence set forth in SEQ ID NO: 453 (or a variant thereof), an HCDR2 comprising the amino acid sequence set forth in SEQ ID NO: 455 (or a variant thereof), and an HCDR3 comprising the amino acid sequence set forth in SEQ ID NO: 457 (or a variant thereof); and an LCVR that comprises: an LCDR1 comprising the amino acid sequence set forth in SEQ ID NO: 461 (or a variant thereof), an LCDR2 comprising the amino acid sequence RNN (or a variant thereof), and an LCDR3 comprising the amino acid sequence set forth in SEQ ID NO: 463 (or a variant thereof)).

In some embodiments, an antigen-binding protein described herein comprises: (1) an HCVR that comprises the amino acid sequence set forth in SEQ ID NO: 1 (or a variant thereof); and an LCVR that comprises the amino acid sequence set forth in SEQ ID NO: 5 (or a variant thereof); (2) an HCVR that comprises the amino acid sequence set forth in SEQ ID NO: 9; and an LCVR that comprises the amino acid sequence set forth in SEQ ID NO: 13 (or a variant thereof); (3) an HCVR that comprises the amino acid sequence set forth in SEQ ID NO: 17; and an LCVR that comprises the amino acid sequence set forth in SEQ ID NO: 21 (or a variant thereof); (4) an HCVR that comprises the amino acid sequence set forth in SEQ ID NO: 25 (or a variant thereof); and an LCVR that comprises the amino acid sequence set forth in SEQ ID NO: 29 (or a variant thereof); (5) an HCVR that comprises the amino acid sequence set forth in SEQ ID NO: 33 (or a variant thereof); and an LCVR that comprises the amino acid sequence set forth in SEQ ID NO: 37 (or a variant thereof); (6) an HCVR that comprises the amino acid sequence set forth in SEQ ID NO: 41 (or a variant thereof); and an LCVR that comprises the amino acid sequence set forth in SEQ ID NO: 45 (or a variant thereof); (7) an HCVR that comprises the amino acid sequence set forth in SEQ ID NO: 49 (or a variant thereof); and an LCVR that comprises the amino acid sequence set forth in SEQ ID NO: 53 (or a variant thereof); (8) an HCVR that comprises the amino acid sequence set forth in SEQ ID NO: 57 (or a variant thereof); and an LCVR that comprises the amino acid sequence set forth in SEQ ID NO: 61 (or a variant thereof); (9) an HCVR that comprises the amino acid sequence set forth in SEQ ID NO: 65 (or a variant thereof); and an LCVR that comprises the amino acid sequence set forth in SEQ ID NO: 69 (or a variant thereof); (10) an HCVR that comprises the amino acid sequence set forth in SEQ ID NO: 73 (or a variant thereof); and an LCVR that comprises the amino acid sequence set forth in SEQ ID NO: 77 (or a variant thereof); (11) an HCVR that comprises the amino acid sequence set forth in SEQ ID NO: 81 (or a variant thereof); and an LCVR that comprises the amino acid sequence set forth in SEQ ID NO: 85 (or a variant thereof); or (12) an HCVR that comprises the amino acid sequence set forth in SEQ ID NO: 89 (or a variant thereof); and an LCVR that comprises the amino acid sequence set forth in SEQ ID NO: 93 (or a variant thereof) (13) an HCVR that comprises the amino acid sequence set forth in SEQ ID NO: 97 (or a variant thereof); and an LCVR that comprises the amino acid sequence set forth in SEQ ID NO: 101 (or a variant thereof); (14) an HCVR that comprises the amino acid sequence set forth in SEQ ID NO: 105 (or a variant thereof); and an LCVR that comprises the amino acid sequence set forth in SEQ ID NO: 109 (or a variant thereof); (15) an HCVR that comprises the amino acid sequence set forth in SEQ ID NO: 113 (or a variant thereof); and an LCVR that comprises the amino acid sequence set forth in SEQ ID NO: 117 (or a variant thereof); (16) an HCVR that comprises the amino acid sequence set forth in SEQ ID NO: 121 (or a variant thereof); and an LCVR that comprises the amino acid sequence set forth in SEQ ID NO: 125 (or a variant thereof); (17) an HCVR that comprises the amino acid sequence set forth in SEQ ID NO: 129 (or a variant thereof); and an LCVR that comprises the amino acid sequence set forth in SEQ ID NO: 133 (or a variant thereof); (18) an HCVR that comprises the amino acid sequence set forth in SEQ ID NO: 137 (or a variant thereof); and an LCVR that comprises the amino acid sequence set forth in SEQ ID NO: 141 (or a variant thereof); (19) an HCVR that comprises the amino acid sequence set forth in SEQ ID NO: 429 (or a variant thereof); and an LCVR that comprises the amino acid sequence set forth in SEQ ID NO: 437 (or a variant thereof); or (20) an HCVR that comprises the amino acid sequence set forth in SEQ ID NO: 451 (or a variant thereof); and an LCVR that comprises the amino acid sequence set forth in SEQ ID NO: 459 (or a variant thereof).

In some embodiments, an antigen-binding protein described herein comprises: (a) a heavy chain that comprises the amino acid sequence set forth in SEQ ID NO: 145 (or a variant thereof), and a light chain that comprises the amino acid sequence set forth in SEQ ID NO: 146 (or a variant thereof); (b) a heavy chain that comprises the amino acid sequence set forth in SEQ ID NO: 147 (or a variant thereof), and a light chain that comprises the amino acid sequence set forth in SEQ ID NO: 148 (or a variant thereof); (c) a heavy chain that comprises the amino acid sequence set forth in SEQ ID NO: 149 (or a variant thereof), and a light chain that comprises the amino acid sequence set forth in SEQ ID NO: 150 (or a variant thereof); (d) a heavy chain that comprises the amino acid sequence set forth in SEQ ID NO: 151 (or a variant thereof), and a light chain that comprises the amino acid sequence set forth in SEQ ID NO: 152 (or a variant thereof); (e) a heavy chain that comprises the amino acid sequence set forth in SEQ ID NO: 153 (or a variant thereof), and a light chain that comprises the amino acid sequence set forth in SEQ ID NO: 154 (or a variant thereof); (f) a heavy chain that comprises the amino acid sequence set forth in SEQ ID NO: 155 (or a variant thereof), and a light chain that comprises the amino acid sequence set forth in SEQ ID NO: 156 (or a variant thereof); (g) a heavy chain that comprises the amino acid sequence set forth in SEQ ID NO: 157 (or a variant thereof), and a light chain that comprises the amino acid sequence set forth in SEQ ID NO: 158 (or a variant thereof); (h) a heavy chain that comprises the amino acid sequence set forth in SEQ ID NO: 159 (or a variant thereof), and a light chain that comprises the amino acid sequence set forth in SEQ ID NO: 160 (or a variant thereof); (i) a heavy chain that comprises the amino acid sequence set forth in SEQ ID NO: 161 (or a variant thereof), and a light chain that comprises the amino acid sequence set forth in SEQ ID NO: 162 (or a variant thereof); (j) a heavy chain that comprises the amino acid sequence set forth in SEQ ID NO: 163 (or a variant thereof), and a light chain that comprises the amino acid sequence set forth in SEQ ID NO: 164 (or a variant thereof); (k) a heavy chain that comprises the amino acid sequence set forth in SEQ ID NO: 165 (or a variant thereof), and a light chain that comprises the amino acid sequence set forth in SEQ ID NO: 166 (or a variant thereof); (l) a heavy chain that comprises the amino acid sequence set forth in SEQ ID NO: 167 (or a variant thereof), and a light chain that comprises the amino acid sequence set forth in SEQ ID NO: 168 (or a variant thereof); (m) a heavy chain that comprises the amino acid sequence set forth in SEQ ID NO: 169 (or a variant thereof), and a light chain that comprises the amino acid sequence set forth in SEQ ID NO: 170 (or a variant thereof); (n) a heavy chain that comprises the amino acid sequence set forth in SEQ ID NO: 171 (or a variant thereof), and a light chain that comprises the amino acid sequence set forth in SEQ ID NO: 172 (or a variant thereof); (o) a heavy chain that comprises the amino acid sequence set forth in SEQ ID NO: 173 (or a variant thereof), and a light chain that comprises the amino acid sequence set forth in SEQ ID NO: 174 (or a variant thereof); (p) a heavy chain that comprises the amino acid sequence set forth in SEQ ID NO: 175 (or a variant thereof), and a light chain that comprises the amino acid sequence set forth in SEQ ID NO: 176 (or a variant thereof); (q) a heavy chain that comprises the amino acid sequence set forth in SEQ ID NO: 177 (or a variant thereof), and a light chain that comprises the amino acid sequence set forth in SEQ ID NO: 178 (or a variant thereof); (r) a heavy chain that comprises the amino acid sequence set forth in SEQ ID NO: 179 (or a variant thereof), and a light chain that comprises the amino acid sequence set forth in SEQ ID NO: 180 (or a variant thereof); (s) a heavy chain that comprises the amino acid sequence set forth in SEQ ID NO: 443 (or a variant thereof), and a light chain that comprises the amino acid sequence set forth in SEQ ID NO: 447 (or a variant thereof); or (t) a heavy chain that comprises the amino acid sequence set forth in SEQ ID NO: 465 (or a variant thereof), and a light chain that comprises the amino acid sequence set forth in SEQ ID NO: 469 (or a variant thereof).

The present disclosure further provides anti-CACNG1 protein-drug conjugates comprising an antibody or antigen-binding fragment thereof that specifically binds to CACNG1 or an antigen-binding fragment thereof comprising: a heavy chain variable region (HCVR) that comprises the HCDR1, HCDR2, and HCDR3 of an HCVR that comprises the amino acid sequence set forth in SEQ ID NO: 1, 9, 17, 25, 33, 41, 49, 57, 65, 73, 81, 89, 97, 105, 113, 121, 129, 137, 429, or 451, and a light chain variable region (LCVR) that comprises the LCDR1, LCDR2, and LCDR3 of an LCVR that comprises the amino acid sequence set forth in SEQ ID NO: 5, 13, 21, 29, 37, 45, 53, 61, 69, 77, 85, 93, 101, 109, 117, 125, 133, 141, 437, or 459.

The present disclosure provides an anti-CACNG1 protein-drug conjugate comprising an isolated antibody or antigen-binding fragment thereof that specifically binds to CACNG1 or an antigenic fragment thereof comprising: (a) a heavy chain variable region (HCVR) that comprises the HCDR1, HCDR2, and HCDR3 of an HCVR that comprises the amino acid sequence set forth in SEQ ID NO: 1, and a light chain variable region (LCVR) that comprises the LCDR1, LCDR2, and LCDR3 of an LCVR that comprises the amino acid sequence set forth in SEQ ID NO: 5; (b) a heavy chain variable region (HCVR) that comprises the HCDR1, HCDR2, and HCDR3 of an HCVR that comprises the amino acid sequence set forth in SEQ ID NO: 9, and a light chain variable region (LCVR) that comprises the LCDR1, LCDR2, and LCDR3 of an LCVR that comprises the amino acid sequence set forth in SEQ ID NO: 13; (c) a heavy chain variable region (HCVR) that comprises the HCDR1, HCDR2, and HCDR3 of an HCVR that comprises the amino acid sequence set forth in SEQ ID NO: 17, and a light chain variable region (LCVR) that comprises the LCDR1, LCDR2, and LCDR3 of an LCVR that comprises the amino acid sequence set forth in SEQ ID NO: 21; (d) a heavy chain variable region (HCVR) that comprises the HCDR1, HCDR2, and HCDR3 of an HCVR that comprises the amino acid sequence set forth in SEQ ID NO: 25, and a light chain variable region (LCVR) that comprises the LCDR1, LCDR2, and LCDR3 of an LCVR that comprises the amino acid sequence set forth in SEQ ID NO: 29; (e) a heavy chain variable region (HCVR) that comprises the HCDR1, HCDR2, and HCDR3 of an HCVR that comprises the amino acid sequence set forth in SEQ ID NO: 33, and a light chain variable region (LCVR) that comprises the LCDR1, LCDR2, and LCDR3 of an LCVR that comprises the amino acid sequence set forth in SEQ ID NO: 37; (f) a heavy chain variable region (HCVR) that comprises the HCDR1, HCDR2, and HCDR3 of an HCVR that comprises the amino acid sequence set forth in SEQ ID NO: 41, and a light chain variable region (LCVR) that comprises the LCDR1, LCDR2, and LCDR3 of an LCVR that comprises the amino acid sequence set forth in SEQ ID NO: 45; (g) a heavy chain variable region (HCVR) that comprises the HCDR1, HCDR2, and HCDR3 of an HCVR that comprises the amino acid sequence set forth in SEQ ID NO: 49, and a light chain variable region (LCVR) that comprises the LCDR1, LCDR2, and LCDR3 of an LCVR that comprises the amino acid sequence set forth in SEQ ID NO: 53; (h) a heavy chain variable region (HCVR) that comprises the HCDR1, HCDR2, and HCDR3 of an HCVR that comprises the amino acid sequence set forth in SEQ ID NO: 57, and a light chain variable region (LCVR) that comprises the LCDR1, LCDR2, and LCDR3 of an LCVR that comprises the amino acid sequence set forth in SEQ ID NO: 61; (i) a heavy chain variable region (HCVR) that comprises the HCDR1, HCDR2, and HCDR3 of an HCVR that comprises the amino acid sequence set forth in SEQ ID NO: 65, and a light chain variable region (LCVR) that comprises the LCDR1, LCDR2, and LCDR3 of an LCVR that comprises the amino acid sequence set forth in SEQ ID NO: 69; (j) a heavy chain variable region (HCVR) that comprises the HCDR1, HCDR2, and HCDR3 of an HCVR that comprises the amino acid sequence set forth in SEQ ID NO: 73, and a light chain variable region (LCVR) that comprises the LCDR1, LCDR2, and LCDR3 of an LCVR that comprises the amino acid sequence set forth in SEQ ID NO: 77; (k) a heavy chain variable region (HCVR) that comprises the HCDR1, HCDR2, and HCDR3 of an HCVR that comprises the amino acid sequence set forth in SEQ ID NO: 81, and a light chain variable region (LCVR) that comprises the LCDR1, LCDR2, and LCDR3 of an LCVR that comprises the amino acid sequence set forth in SEQ ID NO: 85; (l) a heavy chain variable region (HCVR) that comprises the HCDR1, HCDR2, and HCDR3 of an HCVR that comprises the amino acid sequence set forth in SEQ ID NO: 89, and a light chain variable region (LCVR) that comprises the LCDR1, LCDR2, and LCDR3 of an LCVR that comprises the amino acid sequence set forth in SEQ ID NO: 93; (m) a heavy chain variable region (HCVR) that comprises the HCDR1, HCDR2, and HCDR3 of an HCVR that comprises the amino acid sequence set forth in SEQ ID NO: 97, and a light chain variable region (LCVR) that comprises the LCDR1, LCDR2, and LCDR3 of an LCVR that comprises the amino acid sequence set forth in SEQ ID NO: 101; (n) a heavy chain variable region (HCVR) that comprises the HCDR1, HCDR2, and HCDR3 of an HCVR that comprises the amino acid sequence set forth in SEQ ID NO: 105, and a light chain variable region (LCVR) that comprises the LCDR1, LCDR2, and LCDR3 of an LCVR that comprises the amino acid sequence set forth in SEQ ID NO: 109; (o) a heavy chain variable region (HCVR) that comprises the HCDR1, HCDR2, and HCDR3 of an HCVR that comprises the amino acid sequence set forth in SEQ ID NO: 113, and a light chain variable region (LCVR) that comprises the LCDR1, LCDR2, and LCDR3 of an LCVR that comprises the amino acid sequence set forth in SEQ ID NO: 117; (p) a heavy chain variable region (HCVR) that comprises the HCDR1, HCDR2, and HCDR3 of an HCVR that comprises the amino acid sequence set forth in SEQ ID NO: 121, and a light chain variable region (LCVR) that comprises the LCDR1, LCDR2, and LCDR3 of an LCVR that comprises the amino acid sequence set forth in SEQ ID NO: 125; (q) a heavy chain variable region (HCVR) that comprises the HCDR1, HCDR2, and HCDR3 of an HCVR that comprises the amino acid sequence set forth in SEQ ID NO: 129, and a light chain variable region (LCVR) that comprises the LCDR1, LCDR2, and LCDR3 of an LCVR that comprises the amino acid sequence set forth in SEQ ID NO: 133; (r) a heavy chain variable region (HCVR) that comprises the HCDR1, HCDR2, and HCDR3 of an HCVR that comprises the amino acid sequence set forth in SEQ ID NO: 137, and a light chain variable region (LCVR) that comprises the LCDR1, LCDR2, and LCDR3 of an LCVR that comprises the amino acid sequence set forth in SEQ ID NO: 141; (s) a heavy chain variable region (HCVR) that comprises the HCDR1, HCDR2, and HCDR3 of an HCVR that comprises the amino acid sequence set forth in SEQ ID NO: 429, and a light chain variable region (LCVR) that comprises the LCDR1, LCDR2, and LCDR3 of an LCVR that comprises the amino acid sequence set forth in SEQ ID NO: 437; and/or (t) a heavy chain variable region (HCVR) that comprises the HCDR1, HCDR2, and HCDR3 of an HCVR that comprises the amino acid sequence set forth in SEQ ID NO: 451, and a light chain variable region (LCVR) that comprises the LCDR1, LCDR2, and LCDR3 of an LCVR that comprises the amino acid sequence set forth in SEQ ID NO: 459.

The present disclosure also provides anti-CACNG1 protein-drug conjugates comprising an isolated antibody or antigen-binding fragment thereof that specifically binds to CACNG1 or an antigenic fragment thereof comprising: (a) a heavy chain variable region that comprises an HCDR1 that comprises the amino acid sequence set forth in SEQ ID NO: 2, an HCDR2 that comprises the amino acid sequence set forth in SEQ ID NO: 3, and an HCDR3 that comprises the amino acid sequence set forth in SEQ ID NO: 4, and a light chain variable region that comprises an LCDR1 that comprises the amino acid sequence set forth in SEQ ID NO: 6, an LCDR2 that comprises the amino acid sequence set forth in SEQ ID NO: 7, and an LCDR3 that comprises the amino acid sequence set forth in SEQ ID NO: 8; (b) a heavy chain variable region that comprises an HCDR1 that comprises the amino acid sequence set forth in SEQ ID NO: 10, an HCDR2 that comprises the amino acid sequence set forth in SEQ ID NO: 11, and an HCDR3 that comprises the amino acid sequence set forth in SEQ ID NO: 12, and a light chain variable region that comprises an LCDR1 that comprises the amino acid sequence set forth in SEQ ID NO: 14, an LCDR2 that comprises the amino acid sequence set forth in SEQ ID NO: 15, and an LCDR3 that comprises the amino acid sequence set forth in SEQ ID NO: 16; (c) a heavy chain variable region that comprises an HCDR1 that comprises the amino acid sequence set forth in SEQ ID NO: 18, an HCDR2 that comprises the amino acid sequence set forth in SEQ ID NO: 19, and an HCDR3 that comprises the amino acid sequence set forth in SEQ ID NO: 20, and a light chain variable region that comprises an LCDR1 that comprises the amino acid sequence set forth in SEQ ID NO: 22, an LCDR2 that comprises the amino acid sequence set forth in SEQ ID NO: 23, and an LCDR3 that comprises the amino acid sequence set forth in SEQ ID NO: 24; (d) a heavy chain variable region that comprises an HCDR1 that comprises the amino acid sequence set forth in SEQ ID NO: 26, an HCDR2 that comprises the amino acid sequence set forth in SEQ ID NO: 27, and an HCDR3 that comprises the amino acid sequence set forth in SEQ ID NO: 28, and a light chain variable region that comprises an LCDR1 that comprises the amino acid sequence set forth in SEQ ID NO: 30, an LCDR2 that comprises the amino acid sequence set forth in SEQ ID NO: 31, and an LCDR3 that comprises the amino acid sequence set forth in SEQ ID NO: 32; (e) a heavy chain variable region that comprises an HCDR1 that comprises the amino acid sequence set forth in SEQ ID NO: 34, an HCDR2 that comprises the amino acid sequence set forth in SEQ ID NO: 35, and an HCDR3 that comprises the amino acid sequence set forth in SEQ ID NO: 36, and a light chain variable region that comprises an LCDR1 that comprises the amino acid sequence set forth in SEQ ID NO: 38, an LCDR2 that comprises the amino acid sequence set forth in SEQ ID NO: 39, and an LCDR3 that comprises the amino acid sequence set forth in SEQ ID NO: 40; (f) a heavy chain variable region that comprises an HCDR1 that comprises the amino acid sequence set forth in SEQ ID NO: 42, an HCDR2 that comprises the amino acid sequence set forth in SEQ ID NO: 43, and an HCDR3 that comprises the amino acid sequence set forth in SEQ ID NO: 44, and a light chain variable region that comprises an LCDR1 that comprises the amino acid sequence set forth in SEQ ID NO: 46, an LCDR2 that comprises the amino acid sequence set forth in SEQ ID NO: 47, and an LCDR3 that comprises the amino acid sequence set forth in SEQ ID NO: 48; (g) a heavy chain variable region that comprises an HCDR1 that comprises the amino acid sequence set forth in SEQ ID NO: 50, an HCDR2 that comprises the amino acid sequence set forth in SEQ ID NO: 51, and an HCDR3 that comprises the amino acid sequence set forth in SEQ ID NO: 52, and a light chain variable region that comprises an LCDR1 that comprises the amino acid sequence set forth in SEQ ID NO: 54, an LCDR2 that comprises the amino acid sequence set forth in SEQ ID NO: 55, and an LCDR3 that comprises the amino acid sequence set forth in SEQ ID NO: 56; (h) a heavy chain variable region that comprises an HCDR1 that comprises the amino acid sequence set forth in SEQ ID NO: 58, an HCDR2 that comprises the amino acid sequence set forth in SEQ ID NO: 59, and an HCDR3 that comprises the amino acid sequence set forth in SEQ ID NO: 60, and a light chain variable region that comprises an LCDR1 that comprises the amino acid sequence set forth in SEQ ID NO: 62, an LCDR2 that comprises the amino acid sequence set forth in SEQ ID NO: 63, and an LCDR3 that comprises the amino acid sequence set forth in SEQ ID NO: 64; (i) a heavy chain variable region that comprises an HCDR1 that comprises the amino acid sequence set forth in SEQ ID NO: 66, an HCDR2 that comprises the amino acid sequence set forth in SEQ ID NO: 67, and an HCDR3 that comprises the amino acid sequence set forth in SEQ ID NO: 68, and a light chain variable region that comprises an LCDR1 that comprises the amino acid sequence set forth in SEQ ID NO: 70, an LCDR2 that comprises the amino acid sequence set forth in SEQ ID NO: 71, and an LCDR3 that comprises the amino acid sequence set forth in SEQ ID NO: 72; (j) a heavy chain variable region that comprises an HCDR1 that comprises the amino acid sequence set forth in SEQ ID NO: 74, an HCDR2 that comprises the amino acid sequence set forth in SEQ ID NO: 75, and an HCDR3 that comprises the amino acid sequence set forth in SEQ ID NO: 76, and a light chain variable region that comprises an LCDR1 that comprises the amino acid sequence set forth in SEQ ID NO: 78, an LCDR2 that comprises the amino acid sequence set forth in SEQ ID NO: 79, and an LCDR3 that comprises the amino acid sequence set forth in SEQ ID NO: 80; (k) a heavy chain variable region that comprises an HCDR1 that comprises the amino acid sequence set forth in SEQ ID NO: 82, an HCDR2 that comprises the amino acid sequence set forth in SEQ ID NO: 83, and an HCDR3 that comprises the amino acid sequence set forth in SEQ ID NO: 84, and a light chain variable region that comprises an LCDR1 that comprises the amino acid sequence set forth in SEQ ID NO: 86, an LCDR2 that comprises the amino acid sequence set forth in SEQ ID NO: 87, and an LCDR3 that comprises the amino acid sequence set forth in SEQ ID NO: 88; (l) a heavy chain variable region that comprises an HCDR1 that comprises the amino acid sequence set forth in SEQ ID NO: 90, an HCDR2 that comprises the amino acid sequence set forth in SEQ ID NO: 91, and an HCDR3 that comprises the amino acid sequence set forth in SEQ ID NO: 92, and a light chain variable region that comprises an LCDR1 that comprises the amino acid sequence set forth in SEQ ID NO: 94, an LCDR2 that comprises the amino acid sequence set forth in SEQ ID NO: 95, and an LCDR3 that comprises the amino acid sequence set forth in SEQ ID NO: 96; (m) a heavy chain variable region that comprises an HCDR1 that comprises the amino acid sequence set forth in SEQ ID NO: 98, an HCDR2 that comprises the amino acid sequence set forth in SEQ ID NO: 99, and an HCDR3 that comprises the amino acid sequence set forth in SEQ ID NO: 100, and a light chain variable region that comprises an LCDR1 that comprises the amino acid sequence set forth in SEQ ID NO: 102, an LCDR2 that comprises the amino acid sequence set forth in SEQ ID NO: 103, and an LCDR3 that comprises the amino acid sequence set forth in SEQ ID NO: 104; (n) a heavy chain variable region that comprises an HCDR1 that comprises the amino acid sequence set forth in SEQ ID NO: 106, an HCDR2 that comprises the amino acid sequence set forth in SEQ ID NO: 107, and an HCDR3 that comprises the amino acid sequence set forth in SEQ ID NO: 108, and a light chain variable region that comprises an LCDR1 that comprises the amino acid sequence set forth in SEQ ID NO: 110, an LCDR2 that comprises the amino acid sequence set forth in SEQ ID NO: 111, and an LCDR3 that comprises the amino acid sequence set forth in SEQ ID NO: 112; (o) a heavy chain variable region that comprises an HCDR1 that comprises the amino acid sequence set forth in SEQ ID NO: 114, an HCDR2 that comprises the amino acid sequence set forth in SEQ ID NO: 115, and an HCDR3 that comprises the amino acid sequence set forth in SEQ ID NO: 116, and a light chain variable region that comprises an LCDR1 that comprises the amino acid sequence set forth in SEQ ID NO: 118, an LCDR2 that comprises the amino acid sequence set forth in SEQ ID NO: 119, and an LCDR3 that comprises the amino acid sequence set forth in SEQ ID NO: 120; (p) a heavy chain variable region that comprises an HCDR1 that comprises the amino acid sequence set forth in SEQ ID NO: 122, an HCDR2 that comprises the amino acid sequence set forth in SEQ ID NO: 123, and an HCDR3 that comprises the amino acid sequence set forth in SEQ ID NO: 124, and a light chain variable region that comprises an LCDR1 that comprises the amino acid sequence set forth in SEQ ID NO: 126, an LCDR2 that comprises the amino acid sequence set forth in SEQ ID NO: 127, and an LCDR3 that comprises the amino acid sequence set forth in SEQ ID NO: 128; (q) a heavy chain variable region that comprises an HCDR1 that comprises the amino acid sequence set forth in SEQ ID NO: 130, an HCDR2 that comprises the amino acid sequence set forth in SEQ ID NO: 131, and an HCDR3 that comprises the amino acid sequence set forth in SEQ ID NO: 132, and a light chain variable region that comprises an LCDR1 that comprises the amino acid sequence set forth in SEQ ID NO: 134, an LCDR2 that comprises the amino acid sequence set forth in SEQ ID NO: 135, and an LCDR3 that comprises the amino acid sequence set forth in SEQ ID NO: 136; (r) a heavy chain variable region that comprises an HCDR1 that comprises the amino acid sequence set forth in SEQ ID NO: 138, an HCDR2 that comprises the amino acid sequence set forth in SEQ ID NO: 139, and an HCDR3 that comprises the amino acid sequence set forth in SEQ ID NO: 140, and a light chain variable region that comprises an LCDR1 that comprises the amino acid sequence set forth in SEQ ID NO: 142, an LCDR2 that comprises the amino acid sequence set forth in SEQ ID NO: 143, and an LCDR3 that comprises the amino acid sequence set forth in SEQ ID NO: 144; (s) an HCVR that comprises: an HCDR1 comprising the amino acid sequence set forth in SEQ ID NO: 431 (or a variant thereof), an HCDR2 comprising the amino acid sequence set forth in SEQ ID NO: 433 (or a variant thereof), and an HCDR3 comprising the amino acid sequence set forth in SEQ ID NO: 435 (or a variant thereof); and an LCVR that comprises: an LCDR1 comprising the amino acid sequence set forth in SEQ ID NO: 439 (or a variant thereof), an LCDR2 comprising the amino acid sequence YNS (or a variant thereof), and an LCDR3 comprising the amino acid sequence set forth in SEQ ID NO: 441 (or a variant thereof); and/or (t) an HCVR that comprises: an HCDR1 comprising the amino acid sequence set forth in SEQ ID NO: 453 (or a variant thereof), an HCDR2 comprising the amino acid sequence set forth in SEQ ID NO: 455 (or a variant thereof), and an HCDR3 comprising the amino acid sequence set forth in SEQ ID NO: 457 (or a variant thereof); and an LCVR that comprises: an LCDR1 comprising the amino acid sequence set forth in SEQ ID NO: 461 (or a variant thereof), an LCDR2 comprising the amino acid sequence RNN (or a variant thereof), and an LCDR3 comprising the amino acid sequence set forth in SEQ ID NO: 463 (or a variant thereof).

The present disclosure further provides anti-CACNG1 protein-drug conjugates comprising an isolated antibody or antigen-binding fragment thereof that specifically binds to CACNG1 or an antigen-binding fragment thereof comprising a heavy chain variable region that comprises the amino acid sequence set forth in SEQ ID NO: 1, 9, 17, 25, 33, 41, 49, 57, 65, 73, 81, 89, 97, 105, 113, 121, 129, 137, 429, 451 and a light chain variable region that comprises the amino acid sequence set forth in SEQ ID NO: 5, 13, 21, 29, 37, 45, 53, 61, 69, 77, 85, 93, 101, 109, 117, 125, 133, 141, 437, 459.

The present disclosure also provides anti-CACNG1 protein-drug conjugates comprising an isolated antibody or antigen-binding fragment thereof that specifically binds to CACNG1 or an antigen-binding thereof comprising: (a) a heavy chain variable region that comprises the amino acid sequence set forth in SEQ ID NO: 1, and a light chain variable region that comprises the amino acid sequence set forth in SEQ ID NO: 5; (b) a heavy chain variable region that comprises the amino acid sequence set forth in SEQ ID NO: 9, and a light chain variable region that comprises the amino acid sequence set forth in SEQ ID NO: 13; (c) a heavy chain variable region that comprises the amino acid sequence set forth in SEQ ID NO: 17, and a light chain variable region that comprises the amino acid sequence set forth in SEQ ID NO: 21; (d) a heavy chain variable region that comprises the amino acid sequence set forth in SEQ ID NO: 25, and a light chain variable region that comprises the amino acid sequence set forth in SEQ ID NO: 29; (e) a heavy chain variable region that comprises the amino acid sequence set forth in SEQ ID NO: 33, and a light chain variable region that comprises the amino acid sequence set forth in SEQ ID NO: 37; (f) a heavy chain variable region that comprises the amino acid sequence set forth in SEQ ID NO: 41, and a light chain variable region that comprises the amino acid sequence set forth in SEQ ID NO: 45; (g) a heavy chain variable region that comprises the amino acid sequence set forth in SEQ ID NO: 49, and a light chain variable region that comprises the amino acid sequence set forth in SEQ ID NO: 53; (h) a heavy chain variable region that comprises the amino acid sequence set forth in SEQ ID NO: 57, and a light chain variable region that comprises the amino acid sequence set forth in SEQ ID NO: 61; (i) a heavy chain variable region that comprises the amino acid sequence set forth in SEQ ID NO: 65, and a light chain variable region that comprises the amino acid sequence set forth in SEQ ID NO: 69; (j) a heavy chain variable region that comprises the amino acid sequence set forth in SEQ ID NO: 73, and a light chain variable region that comprises the amino acid sequence set forth in SEQ ID NO: 77; (k) a heavy chain variable region that comprises the amino acid sequence set forth in SEQ ID NO: 81, and a light chain variable region that comprises the amino acid sequence set forth in SEQ ID NO: 85; (l) a heavy chain variable region that comprises the amino acid sequence set forth in SEQ ID NO: 89, and a light chain variable region that comprises the amino acid sequence set forth in SEQ ID NO: 93; (m) a heavy chain variable region that comprises the amino acid sequence set forth in SEQ ID NO: 97, and a light chain variable region that comprises the amino acid sequence set forth in SEQ ID NO: 101; (n) a heavy chain variable region that comprises the amino acid sequence set forth in SEQ ID NO: 105, and a light chain variable region that comprises the amino acid sequence set forth in SEQ ID NO: 109; (o) a heavy chain variable region that comprises the amino acid sequence set forth in SEQ ID NO: 113, and a light chain variable region that comprises the amino acid sequence set forth in SEQ ID NO: 117; (p) a heavy chain variable region that comprises the amino acid sequence set forth in SEQ ID NO: 121, and a light chain variable region that comprises the amino acid sequence set forth in SEQ ID NO: 125; (q) a heavy chain variable region that comprises the amino acid sequence set forth in SEQ ID NO: 129, and a light chain variable region that comprises the amino acid sequence set forth in SEQ ID NO: 133; (r) a heavy chain variable region that comprises the amino acid sequence set forth in SEQ ID NO: 137, and a light chain variable region that comprises the amino acid sequence set forth in SEQ ID NO: 141; (s) a heavy chain variable region that comprises the amino acid sequence set forth in SEQ ID NO: 429, and a light chain variable region that comprises the amino acid sequence set forth in SEQ ID NO: 437; and/or (t) a heavy chain variable region that comprises the amino acid sequence set forth in SEQ ID NO: 451, and a light chain variable region that comprises the amino acid sequence set forth in SEQ ID NO: 459.

The present disclosure provides anti-CACNG1 protein-drug conjugates comprising an isolated antibody or antigen-binding fragment thereof that specifically binds to CACNG1 or an antigen-binding fragment thereof comprising a heavy chain that comprises the amino acid sequence set forth in SEQ ID NO: 145, 147, 149, 151, 153, 155, 157, 159, 161, 163, 165, 167, 169, 171, 173, 175, 177, 179, 443, 465, and a light chain that comprises the amino acid sequence set forth in SEQ ID NO: 146, 148, 150, 152, 154, 156, 158, 160, 162, 164, 166, 168, 170, 172, 174, 176, 178, 180, 447, 469.

The present disclosure also provides anti-CACNG1 protein-drug conjugates comprising an isolated antibody or antigen-binding fragment thereof that specifically binds to CACNG1 or an antigen-binding fragment thereof comprising: (a) a heavy chain that comprises the amino acid sequence set forth in SEQ ID NO: 145, and a light chain that comprises the amino acid sequence set forth in SEQ ID NO: 146; (b) a heavy chain that comprises the amino acid sequence set forth in SEQ ID NO: 147, and a light chain that comprises the amino acid sequence set forth in SEQ ID NO: 148; (c) a heavy chain that comprises the amino acid sequence set forth in SEQ ID NO: 149, and a light chain that comprises the amino acid sequence set forth in SEQ ID NO: 150; (d) a heavy chain that comprises the amino acid sequence set forth in SEQ ID NO: 151, and a light chain that comprises the amino acid sequence set forth in SEQ ID NO: 152; (e) a heavy chain that comprises the amino acid sequence set forth in SEQ ID NO: 153, and a light chain that comprises the amino acid sequence set forth in SEQ ID NO: 154; (f) a heavy chain that comprises the amino acid sequence set forth in SEQ ID NO: 155, and a light chain that comprises the amino acid sequence set forth in SEQ ID NO: 156; (g) a heavy chain that comprises the amino acid sequence set forth in SEQ ID NO: 157, and a light chain that comprises the amino acid sequence set forth in SEQ ID NO: 158; (h) a heavy chain that comprises the amino acid sequence set forth in SEQ ID NO: 159, and a light chain that comprises the amino acid sequence set forth in SEQ ID NO: 160; (i) a heavy chain that comprises the amino acid sequence set forth in SEQ ID NO: 161, and a light chain that comprises the amino acid sequence set forth in SEQ ID NO: 162; (j) a heavy chain that comprises the amino acid sequence set forth in SEQ ID NO: 163, and a light chain that comprises the amino acid sequence set forth in SEQ ID NO: 164; (k) a heavy chain that comprises the amino acid sequence set forth in SEQ ID NO: 165, and a light chain that comprises the amino acid sequence set forth in SEQ ID NO: 166; (l) a heavy chain that comprises the amino acid sequence set forth in SEQ ID NO: 167, and a light chain that comprises the amino acid sequence set forth in SEQ ID NO: 168; (m) a heavy chain that comprises the amino acid sequence set forth in SEQ ID NO: 169, and a light chain that comprises the amino acid sequence set forth in SEQ ID NO: 170; (n) a heavy chain that comprises the amino acid sequence set forth in SEQ ID NO: 171, and a light chain that comprises the amino acid sequence set forth in SEQ ID NO: 172; (o) a heavy chain that comprises the amino acid sequence set forth in SEQ ID NO: 173, and a light chain that comprises the amino acid sequence set forth in SEQ ID NO: 174; (p) a heavy chain that comprises the amino acid sequence set forth in SEQ ID NO: 175, and a light chain that comprises the amino acid sequence set forth in SEQ ID NO: 176; (q) a heavy chain that comprises the amino acid sequence set forth in SEQ ID NO: 177, and a light chain that comprises the amino acid sequence set forth in SEQ ID NO: 178; (r) a heavy chain that comprises the amino acid sequence set forth in SEQ ID NO: 179, and a light chain that comprises the amino acid sequence set forth in SEQ ID NO: 180; (s) a heavy chain that comprises the amino acid sequence set forth in SEQ ID NO: 443 (or a variant thereof), and a light chain that comprises the amino acid sequence set forth in SEQ ID NO: 447 (or a variant thereof); and/or (t) a heavy chain that comprises the amino acid sequence set forth in SEQ ID NO: 465 (or a variant thereof), and a light chain that comprises the amino acid sequence set forth in SEQ ID NO: 469 (or a variant thereof).

In some embodiments, the antigen-binding protein binds to the same epitope on human CACNG1 as an antibody comprising an HCVR/LCVR amino acid sequence pair as set forth in Table 1-1.

As provided herein, an anti-CACNG1 protein-drug conjugate disclosed herein may comprise an anti-CACNG1 scFv comprising an optional signal peptide (e.g., mROR signal sequence), connected to an scFv (e.g., including a VL and a VH optionally connected by a linker), connected to an optional linker, connected to a molecular cargo. In various embodiments, the optional signal peptide is, for example, the signal peptide from Mus musculus Ror 1 (e.g., comprising or consisting of the amino acids MHRPRRRGTRPPPLALLAALLLAARGΔΔCt A (SEQ ID NO: 363)).

In some embodiments, an anti-CACNG1 scFv described herein, in VL-(Gly4Ser)3-VH (“GGGGSGGGGSGGGGS” disclosed as SEQ ID NO: 423) format, comprises an amino acid sequence as set forth in Table 1-1. In other embodiments, the present disclosure includes scFvs that are in the format VH-(Gly4Ser)3-VL (“GGGGSGGGGSGGGGS” disclosed as SEQ ID NO: 423). Optionally, an anti-CACNG1 scFv of the present disclosure further includes a tag sequence LLQGSG (SEQ ID NO: 364) and/or HHHHHH (SEQ ID NO: 365). The tag sequence LLQGSG (SEQ ID NO: 364) or HHHHHH (SEQ ID NO: 365) may be included at the N-terminus and/or C-terminus of the anti-CACNG1 scFv. In one embodiment, an anti-CACNG1 scFv of the present disclosure further includes an N-terminal LLQGSG (SEQ ID NO: 364) and/or a C-terminal HHHHHH (SEQ ID NO: 365).

In some embodiments, the CACNG1 binding protein described herein comprises a humanized antibody or antigen binding fragment thereof, human antibody or antigen binding fragment thereof, murine antibody or antigen binding fragment thereof, chimeric antibody or antigen binding fragment thereof, monoclonal antibody or antigen binding fragment thereof (e.g., monovalent Fab′, divalent Fab2, F(ab)′3 fragments, single-chain variable fragment (scFv), bis-scFv, (scFv)₂, diabody, bivalent antibody, one-armed antibody, minibody, nanobody, triabody, tetrabody, disulfide stabilized Fv protein (dsFv), single-domain antibody (sdAb), Ig NAR, camelid antibody or antigen binding fragment thereof, single heavy chain antibody, bispecific antibody or binding fragment thereof, (e.g., bisscFv, or a bi-specific T-cell engager (BiTE)), trispecific antibody (e.g., F(ab)′3 fragments or a triabody), or a chemically modified derivative thereof. In some embodiments, the CACNG1 binding protein described herein comprises a fragment antigen-binding region (Fab). In some embodiments, the anti-CACNG1 antigen-binding protein can be bivalent. In some embodiments, the anti-CACNG1 antigen-binding protein can be monovalent (e.g., one-arm antibody).

The term “humanized antibody”, as used herein, includes antibodies in which CDR sequences derived from the germline of another mammalian species, such as a mouse, have been grafted onto human framework sequences, or otherwise modified to increase their similarity to antibody variants produced naturally in humans.

In some cases, the CACNG1-binding protein is an antibody which comprises one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcyR, antibody-dependent cell-mediated cytotoxicity (ΔΔCt CC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

The anti-CACNG1 antibodies and antigen-binding fragments described herein may be modified after translation, e.g., glycosylated.

For example, antibodies and antigen-binding fragments described herein may be glycosylated (e.g., N-glycosylated and/or O-glycosylated) or aglycosylated. Typically, antibodies and antigen-binding fragments are glycosylated at the conserved residue N297 of the IgG Fc domain. Some antibodies and fragments include one or more additional glycosylation sites in a variable region. In an embodiment, the glycosylation site is in the following context: FN₂₉₇S or YN₂₉₇S.

In an embodiment, said glycosylation is any one or more of three different N-glycan types: high mannose, complex and/or hybrid that are found on IgGs with their respective linkage. Complex and hybrid types exist with core fucosylation, addition of a fucose residue to the innermost N-acetylglucosamine, and without core fucosylation.

In some cases, the CACNG1-binding protein is an aglycosylated antibody, i.e., an antibody that does not comprise a glycosylation sequence that might interfere with a transglutamination reaction, for instance an antibody that does not have a saccharide group at N297 on one or more heavy chains according to the EU numbering system (or position N180 with reference to the amino acid sequence ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGGP SVFLFPPKPKDTLMISRTPEVTCVVVDVSHEDPEVKFNWYVDGVEVHNAKTKPREEQYNS TYRVVSVLTVLHQDWLNGKEYKCKVSNKALPAPIEKTISKAKGQPREPQVYTLPPSRDELT KNQVSLTCLVKGFYPSDIAVEWESNGQPENNYKTTPPVLDSDGSFFLYSKLTVDKSRWQQ GNVFSCSVMHEALHNHYTQKSLSLSPGK (SEQ ID NO: 472). In particular embodiments, an antibody heavy chain has an N297 mutation (or an N180 mutation with reference to the amino acid sequence of SEQ ID NO: 472). In particular embodiments, an antibody heavy chain has an N297Q or an N297D mutation (or an N180Q or N180D mutation with reference to the amino acid sequence of SEQ ID NO: 472). The N-linked glycan found at position 297 can be found as a core structure, common to all IgG found in human beings and rodents. Antibodies comprising such above-described mutations can be prepared by site-directed mutagenesis to remove or disable a glycosylation sequence or by site-directed mutagenesis to insert a glutamine residue at site apart from any interfering glycosylation site or any other interfering structure. Such antibodies also can be isolated from natural or artificial sources. Aglycosylated antibodies also include antibodies comprising a T299 or S298P or other mutations, or combinations of mutations that result in a lack of glycosylation.

In some cases, the antigen-binding protein is a deglycosylated antibody, i.e., an antibody in which a saccharide group at is removed to facilitate transglutaminase-mediated conjugation. Saccharides include, but are not limited to, N-linked oligosaccharides. In some embodiments, deglycosylation is performed at residue N297. In some embodiments, deglycosylation is performed at residue N180 with reference to the amino acid sequence of SEQ ID NO: 472. In some embodiments, removal of saccharide groups is accomplished enzymatically, included but not limited to via PNGase.

In an embodiment, an antibody or fragment described herein is afucosylated.

The antibodies and antigen-binding fragments described herein may also be post-translationally modified in other ways including, for example: Glu or Gln cyclization at N-terminus; Loss of positive N-terminal charge; Lys variants at C-terminus; Deamidation (Asn to Asp); Isomerization (Asp to isoAsp); Deamidation (Gln to Glu); Oxidation (Cys, His, Met, Tyr, Trp); and/or Disulfide bond heterogeneity (Shuffling, thioether and trisulfide formation).

In some embodiments, an antibody disclosed herein comprises Q295 which can be native to the antibody heavy chain sequence. In some embodiments, an antibody heavy chain disclosed herein may comprise Q295. In some embodiments, an antibody heavy chain disclosed herein may comprise Q295 and an amino acid substitution N297D.

According to certain embodiments of the present disclosure, anti-CACNG1 antibodies and antigen-binding fragments are provided comprising an Fc domain comprising one or more mutations which enhance or diminish antibody binding to the FcRn receptor, e.g., at acidic pH as compared to neutral pH. For example, the present disclosure includes anti-CACNG1 antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
   and/or a modification at position:
428 and/or 433 (e.g., H/L/R/S/P/Q or K), and/or
434 (e.g., A, W, H, F or Y);
   and/or a modification at position:
250 and/or 428;
   and/or a modification at position:
307 or 308 (e.g., 308F, V308F), and/or 434. In an embodiment, the modification comprises:
a 428L (e.g., M428L) and 434S (e.g., N434S) modification;
a 428L, 2591 (e.g., V2591), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present disclosure includes anti-CACNG1 antibodies comprising an Fc domain comprising one or more pairs or groups of mutations selected from the group consisting of:
250Q and 248L (e.g., T250Q and M248L);
252Y, 254T and 256E (e.g., M252Y, S254T and T256E);
2571 and 3111 (e.g., P2571 and Q3111);
2571 and 434H (e.g., P2571 and N434H);
376V and 434H (e.g., D376V and N434H);
307A, 380A and 434A (e.g., T307A, E380A and N434A);
428L and 434S (e.g., M428L and N434S); and
433K and 434F (e.g., H433K and N434F).

In yet another embodiment, the modification comprises a 265A (e.g., D265A) and/or a 297A (e.g., N297A) modification.

In an embodiment, the heavy chain constant domain is gamma-4 comprising an S228P and/or S108P mutation. See Angal et al., A single amino acid substitution abolishes the heterogeneity of chimeric mouse/human (IgG4) antibody, Mol Immunol. 1993 January; 30(1):105-108.

In some embodiments, an anti-CACNG1 antibody and antigen-binding fragment thereof is provided comprising an Fc domain comprising one or more mutations which enhance or diminish antibody binding to the FcRn receptor, e.g., at acidic pH as compared to neutral pH. For example, antibodies as described herein may comprise a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an in-crease in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modifica-tion; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, an anti-CACNG1 antibody and antigen-binding fragment as described herein may comprise an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F).

All possible combinations of the foregoing Fc domain mutations, and other mutations within the antibody variable domains disclosed herein, are contemplated within the scope of the present disclosure.

The anti-CACNG1 antibodies described herein may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ΔΔCt CC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcyR).

In certain embodiments, the modified Fc domain is a variant IgG1 Fc or a variant IgG4 Fc comprising a substitution in the hinge region. For example, a modified Fc for use in the context of the present disclosure may comprise a variant IgG1 Fc wherein at least one amino acid of the IgG1 Fc hinge region is replaced with the corresponding amino acid from the IgG2 Fc hinge region. Alternatively, a modified Fc for use in the context of the present disclosure may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present disclosure are set forth in US Patent Application Publication No. 2014/0243504, the disclosure of which is hereby incorporated by reference in its entirety, as well as any functionally equivalent variants of the modified Fc regions set forth therein.

Also provided herein are antigen-binding proteins (e.g., antibodies or antigen-binding fragments), comprising an HCVR set forth herein and a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies provided herein comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. See, e.g., WO2014/022540.

Other modified Fc domains and Fc modifications that can be used in the context of the present disclosure include any of the modifications as set forth in US2014/0171623; U.S. Pat. No. 8,697,396; US2014/0134162; WO2014/043361, the disclosures of which are hereby incorporated by reference in their entireties. Methods of constructing antibodies or other antigen-binding fusion proteins comprising a modified Fc domain as described herein are known in the art.

Antigen-binding molecules having amino acid sequences that vary from those of the exemplary molecules disclosed herein but that retain the ability to bind CACNG1 are also described herein. Such variant molecules may comprise one or more additions, deletions, or substitutions of amino acids when compared to parent sequence, but exhibit biological activity that is essentially equivalent to that of the described antigen-binding molecules.

Antigen-binding molecules that are bioequivalent to any of the exemplary antigen-binding molecules set forth herein are also described. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or re-move glycosylation.

In some embodiments, antigen-binding molecules as described herein bind to human CACNG1 but not to CACNG1 from other species. Also described herein are antigen-binding molecules that bind to human CACNG1 and to CACNG1 from one or more non-human species.

In some embodiments, antigen-binding molecules as described herein that bind to human CACNG1 may bind, or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CACNG1.

The present disclosure provides a vessel (e.g., a plastic or glass vial, e.g., with a cap or a chromatography column, hollow bore needle or a syringe cylinder) comprising antigen-binding proteins (e.g., an anti-CACNG1 antibodies or antigen-binding fragments thereof described herein) and/or anti-CACNG1 protein-drug conjugates, e.g., CACNG1-binding protein-drug conjugates or anti-CACNG1 Fab-drug conjugates described herein.

The present disclosure also provides an injection device comprising an antigen-binding protein (e.g., an anti-CACNG1 antibody or antigen-binding fragment thereof described herein) and/or an anti-CACNG1 protein-drug conjugate, e.g., anti-CACNG1 scFv-drug conjugates or anti-CACNG1 Fab-drug conjugates described herein, or a pharmaceutical composition thereof. The injection device may be packaged into a kit. An injection device is a device that introduces a substance into the body of a subject via a parenteral route, e.g., intramuscular, subcutaneous or intravenous. For example, an injection device may be a syringe or an auto-injector (e.g., pre-filled with the pharmaceutical formulation) which, for example, includes a cylinder or barrel for holding fluid to be injected (e.g., comprising the antibody or fragment or a pharmaceutical formulation thereof), a needle for piecing skin, blood vessels or other tissue for injection of the fluid; and a plunger for pushing the fluid out of the cylinder and through the needle bore and into the body of the subject.

The present disclosure provides methods for administering an anti-antigen-binding protein, e.g., antibody or antigen-binding fragment thereof to a subject, comprising introducing the protein or a pharmaceutical formulation thereof into the body of the subject. For example, in an embodiment, the method comprises piercing the body of the subject, e.g., with a needle of a syringe, and injecting the antigen-binding protein or a pharmaceutical formulation thereof into the body of the subject, e.g., into the eye, vein, artery, muscular tissue or subcutis of the subject.

The present disclosure further provides methods for delivering a molecular cargo, wherein the molecular cargo is conjugated to, e.g., an antigen-binding protein described herein, e.g., an anti-CACNG1 scFv or an anti-CACNG1 Fab described herein, to a targeted tissue (e.g., muscle tissue) or a target cell (e.g., myofiber) in a subject, comprising introducing the protein-drug conjugate into the body of the subject (e.g., a human), for example, parenterally (e.g., via intramuscular, subcutaneous, or intravenous injection). For example, the method comprises piercing the body of the subject with a needle of a syringe and injecting the protein-drug conjugate into the body of the subject, e.g., into the skeletal muscle of the subject. For example, the protein-drug conjugate may be introduced into the subject via intramuscular injection into the skeletal muscle.

### Molecular Cargoes

In some aspects, the present disclosure includes methods and compositions for delivering a conjugated molecular cargo to a cell or tissue. In certain aspects the antigen-binding protein that binds specifically to Calcium Voltage-Gated Channel Auxiliary Subunit Gamma 1 (CACNG1) disclosed herein, e.g., an antibody or an antigen-binding fragment thereof (e.g., an scFv), may be conjugated (e.g., covalently conjugated) to the molecular cargo (e.g., a polynucleotide, a polypeptide, a small molecule, a liposome, or an LNP disclosed herein). Accordingly, the present disclosure provides antibody-drug conjugates (ADCs) comprising an anti-hCACNG1 antibody or an antigen-binding fragment thereof conjugated to a “drug” (e.g., a polynucleotide, a polypeptide, a small molecule, a liposome, or an LNP disclosed herein).

In general terms, an ΔΔCt C disclosed herein can comprise: A-[L-P]y, in which A is an antigen-binding molecule, e.g. an anti-hCACNG1 antibody, or an antigen-binding fragment thereof (e.g., a fragment comprising at least an HCDR3 selected from any of the HCDR3 amino acid sequences listed in Table 1-1), L is a linker, P is the payload or molecular cargo, and y is an integer from 1 to 30.

As used herein, the term “molecular cargo” or a “payload” refers to a molecule that operates to effect a biological outcome. As a non-limiting example, the molecular cargo may operate to modulate the transcription of a DNA sequence, to modulate the expression of a protein, or to modulate the activity of a protein, to delete or disrupt an endogenous gene (or fragment thereof), to achieve an enzymatic activity, to supplement or replace a deficient endogenous protein, to insert an exogenous gene (or fragment thereof), or to replace an endogenous gene (or fragment thereof) with an exogenous gene (or fragment thereof). In various embodiments, the molecular cargo may comprise a polynucleotide. In various embodiments, the molecular cargo may comprise a polypeptide. In various embodiments, the molecular cargo comprises a lipid nanoparticle (LNP), liposome, or non-lipid nanoparticle described herein, which optionally comprises one or more polynucleotides and/or protein molecules. In various embodiments, the molecular cargo may comprise a small molecule.

In some embodiments, the anti-CACNG1 antibody or an antigen-binding fragment thereof disclosed herein may be used, for example, to deliver the conjugated molecular cargo to a tissue or a cell that expresses CACNG1 (e.g., skeletal muscle tissue or myofiber) for diagnosing and or treating a disease (e.g., atrophy and/or myotonic dystrophy). In some embodiments, the molecular cargoes conjugated to the anti-CACNG1 antibody or antigen-binding fragment thereof may be taken up by, e.g., myofibers, via binding to the CACNG1, which may be endocytosed. In some embodiments, the anti-CACNG1 antibody or an antigen-binding fragment thereof described herein can exhibit superior activity, e.g., in delivering a molecular cargo into a target tissue (e.g., skeletal muscle tissue) or a target cell (e.g., a myofiber).

In some embodiments, the molecular cargo comprises a polynucleotide molecule. The terms “polynucleotide” and “nucleic acid” are used interchangeably herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with optional substitutions, e.g., methoxy or 2′ halide substitutions. In some embodiments, polynucleotides up to about 30 nucleotides in length can be referred to herein as an “oligonucleotide”. Oligonucleotides may be of a variety of different lengths, e.g., depending on the form. In some embodiments, an oligonucleotide is 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the oligonucleotide is 8 to 30 nucleotides in length, 10 to 15 nucleotides in length, 10 to 20 nucleotides in length, 15 to 25 nucleotides in length, 21 to 23 nucleotides in lengths.

In some embodiments, the molecular cargo comprises a polypeptide molecule. The terms “polypeptide” and “protein” used interchangeably herein encompass native or artificial proteins, protein fragments and polypeptide analogs of a protein sequence. A polypeptide or protein may be monomeric or polymeric.

In some embodiments, a protein cargo described herein can include biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins may be produced using recombinant cell-based production systems, such as the insect baculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation,” 28 Biotechnol Genet Eng Rev. 147-75 (2012).

In some embodiments, the molecular cargo described herein may comprise a carrier, such as a liposome or lipid nanoparticle (LNP). A lipid particle, e.g., a liposome or lipid nanoparticle disclosed herein, may include a lipid formulation that can be used to deliver a therapeutic nucleic acid (e.g., gRNA) to a target site of interest (e.g., cell, tissue, organ, and the like). Without wishing to be bound by theory, carriers may be used, e.g., as a means for delivery of a polynucleotide disclosed herein and/or a protein disclosed herein. In some embodiments, a carrier (e.g., liposome or LNP) may be useful for the delivery of a nucleic acid (e.g., DNA or RNA), protein (e.g., RNA-guided DNA binding agent), or a combination thereof. By way of a non-limiting example, a carrier (e.g., liposome or LNP) may be used to deliver various components of a gene editing system, for example, a CRISPR/Cas system or additional gene editing systems described herein.

In some embodiments, the molecular cargo comprises a small molecule. A small molecule (SM) can permeably enter or diffuse into cells. For example, without limitation, once a conjugate comprising a small molecule (e.g., a bioactive small molecule) described herein internalizes to inside a cell, the linker of the conjugate can be cleaved to release the bioactive small molecule which can then modulate intracellular bio-responses, such as, but not limited to, binding to a nuclear receptor (e.g., dihydrotestosterone (DHT) binding to androgen receptor, budesonide binding to glucocorticoid receptor) or other proteins. Thus, once inside a cell, a small molecule can affect other various molecules, such as proteins therein. This is different from many large molecular weight molecules such as antibodies. As a non-limiting example, a small molecule of the present disclosure can be, e.g., an androgen, (e.g., testosterone or a biologically equivalent variant thereof or dihydrotestosterone (DHT)), a glucocorticoid (e.g., budesonide), a β2-adrenergic receptor agonist, rapamycin or an analog thereof, a MAPK inhibitor, or a histone deacetylase inhibitor. In some embodiments, the small molecule can be testosterone or a biologically equivalent variant thereof. In some embodiments, the small molecule can be dihydrotestosterone (DHT). In some embodiments, the small molecule can be a glucocorticoid, e.g., budesonide, or a biologically equivalent variant thereof. In various embodiments, a small molecule can comprise a detectable biosensor or a radioactive isotope. In some embodiments, a radioactive isotope comprises a radionuclide. An antigen-binding protein and a small molecule described herein can be conjugated in some instances via a valine-citrulline para-aminobenzylcarbamate (VC-PAB) and/or a glutamic acid-valine-citrulline para-aminobenzylcarbamate (EVC-PAB) linker. In certain embodiments, an antigen-binding protein described herein can be conjugated to a small molecule, detectable biosensor, and/or a radioactive isotope via a valine-citrulline para-aminobenzylcarbamate (VC-PAB) and/or a glutamic acid-valine-citrulline para-aminobenzylcarbamate (EVC-PAB) linker.

Exemplary molecular cargoes are described in further detail herein; however, it should be appreciated that the exemplary molecular cargoes provided herein are not intended to be limiting.

### Polynucleotide Molecules

Non-limiting examples of polynucleotide molecules that are useful as molecular cargoes in the protein-drug conjugates of the present disclosure include, but are not limited to, interfering nucleic acids (e.g., shRNAs, siRNAs, microRNAs, antisense oligonucleotides, gapmers), mixmers, ribozymes, phosphorodiamidite morpholinos, peptide nucleic acids, aptamers, and guide nucleic acids (e.g., Cas9 guide RNAs), mRNAs, etc. In various embodiments, a polynucleotide may comprise one or more modified nucleotides. In various embodiments, a polynucleotide may comprise one or more modified inter-nucleotide linkage. Polynucleotides may be single-stranded or double-stranded.

In some embodiments, the molecular cargo comprises at least one polynucleotide molecule. In some embodiments, the molecular cargo comprises at least 2, at least 3, at least 4, at least 5, or at least 10 polynucleotide molecules.

In some embodiments, the polynucleotide molecule is DNA. In some embodiments, the polynucleotide molecule is RNA.

In various embodiments, a polynucleotide described herein (e.g., interfering nucleic acid or guide RNA) may comprise a region of complementarity to a target nucleic acid which can be in the range of 8 to 15, 8 to 30, 8 to 40, or 10 to 50, or 5 to 50, or 5 to 40 nucleotides in length. In certain embodiments, a region of complementarity of a polynucleotide to a target nucleic acid may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity may be complementary with at least 10 consecutive nucleotides of a target nucleic acid. In some embodiments, a polynucleotide may contain 1, 2, 3, 4 or 5 base mismatches compared to the portion of the consecutive nucleotides of target nucleic acid. In some embodiments the polynucleotide may have up to 3 mismatches over 15 bases, or up to 4 mismatches over 10 bases. In some embodiments, the polynucleotide is complementary (e.g., at least 80%, at least 85% at least 90%, at least 95%, or 100%) to a target sequence of any one of the polynucleotides of the present disclosure. In various embodiments, such target sequence may be 100% complementary to the polynucleotide described herein. In some embodiments, any one or more of the thymine bases (T's) in any one of the polynucleotides described herein may be uracil bases (U's), and/or any one or more of the U's may be T's. A target sequence described herein may comprise a sequence of nucleic acid in a target gene that has complementarity to the guide sequence of the gRNA. The interaction of the target sequence and the guide sequence directs an RNA-guided DNA-binding agent (e.g., Cas protein) to bind, and potentially nick or cleave (depending on the activity of the agent), within the target sequence.

The polynucleotides described herein may be modified, e.g., comprise a modified nucleotide, a modified internucleoside linkage, and/or a modified sugar moiety, or combinations thereof. In addition, polynucleotides can possess one or more of the following properties: have improved cell uptake compared to unmodified polynucleotides; are not toxic to cells or mammals are not immune stimulatory; avoid pattern recognition receptors do not mediate alternative splicing; are nuclease resistant; have improved endosomal exit internally in a cell; or minimizes TLR stimulation. Any of the various modified chemistries or formats of polynucleotides disclosed herein may be combined with together. As a non-limiting example, one, two, three, four, five, six, seven, eight or more different types of modifications may be included within the same polynucleotide.

In various embodiments, particular nucleotide modification(s) may be used that render a polynucleotide into which the modification(s) are incorporated more resistant to nuclease digestion than the native oligoribonucleotide or oligodeoxynucleotide molecules, such modified polynucleotides survive intact for a longer time than unmodified polynucleotides. Exemplary modified polynucleotides include those comprising modified backbones, for example, modified internucleoside linkages such as, methyl phosphonates, phosphotriesters, phosphorothioates short chain alkyl or cycloalkyl intersugar linkages heterocyclic intersugar linkages or short chain heteroatomic or. As such, polynucleotides described herein may be stabilized against nucleolytic degradation, e.g., via incorporation of a modification, e.g., a nucleotide modification.

In various embodiments, a polynucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, or 2 to 45, nucleotides of the polynucleotide may be modified nucleotides. The polynucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the polynucleotide can be modified nucleotides. In some embodiments, the polynucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the polynucleotide are modified nucleotides. In some embodiments, the polynucleotides can have every nucleotide except 1, 2, 3, 4, 5, 6, 7, 8, 9, 10, 11, 12, 13, 14, or 15 nucleotides modified.

In various embodiments, the polynucleotide disclosed herein may comprise at least one nucleoside, e.g., modified at the 2′ position of the sugar. In some embodiments, all of the nucleosides in the polynucleotide are 2′-modified nucleosides. In some embodiments, a polynucleotide comprises at least one 2′-modified nucleoside.

In various embodiments, the polynucleotide disclosed herein may one or more non-bicyclic 2′-modified nucleosides, e.g., 2′-O-dimethylaminoethyloxyethyl (2′-O-DMAEOE)2′-O-methyl (2′-O-Me), 2′-O-dimethylaminoethyl (2′-O-DMAOE), 2′-O-methoxyethyl (2′-MOE), 2′-deoxy, 2′-O—N-methylacetamido (2′-O—NMA) modified nucleoside, 2′-fluoro (2′-F), 2′-O-aminopropyl (2′-O-AP), or 2′-O-dimethylaminopropyl (2′-O-DMAP).

In some embodiments, the polynucleotide of the present disclosure may comprise one or more 2′-4′ bicyclic nucleosides in which the ribose ring may comprise a bridge moiety, e.g., connecting two atoms in the ring (e.g., connecting the 2′-O atom to the 4′-C atom via an ethylene (ENA) bridge, a methylene (LNA) bridge, or a (S)-constrained ethyl (cEt) bridge). Non-limiting examples of ENAs are disclosed in PCT Publication No. WO 2005/042777; Morita et al., Nucleic Acid Res., Suppl 1:241-242, 2001; Koizumi, Curr. Opin. Mol. Ther., 8:144-149, 2006, Surono et al., Hum. Gene Ther., 15:749-757, 2004; and Horie et al., Nucleic Acids Symp. Ser (Oxf), 49:171-172, 2005; the disclosures of which are incorporated herein by reference in their entireties. Non-limiting examples of LNAs are disclosed in PCT Patent Application Publication No. WO2008/043753, the contents of which are incorporated herein by reference in its entirety. Non-limiting examples of cEt are disclosed in in U.S. Pat. Nos. 7,569,686, 7,101,993, and 7,399,845 each of which is herein incorporated by reference in its entirety.

In various embodiments, the polynucleotide described herein may comprise a modified nucleoside disclosed in, for example, U.S. Pat. Nos. 8,022,193; 7,569,686; 7,399,845; 7,741,457; 7,335,765; 7,816,333; 8,957,201; 7,314,923, the entire contents of each of which are incorporated herein by reference for all purposes.

In various embodiments, the polynucleotide comprises at least one modified nucleoside that results in an increase in Tm of the polynucleotide in a range of 1° C. to 10° C. compared with a polynucleotide that does not have the at least one modified nucleoside. The polynucleotide may have a plurality of modified nucleosides that result in a total increase in Tm of the polynucleotide in a range of 2° C., 3° C., 4° C., 5° C., 6° C., 7° C., 8° C., 9° C., 10° C., 15° C., 20° C., 25° C., 30° C., 35° C., 40° C., 45° C., 50° C., 55° C., 60° C. or more as compared to a polynucleotide which does not have the modified nucleoside.

In some embodiments, the polynucleotide may comprise a mix of nucleosides of different kinds. A polynucleotide may comprise a mix of deoxyribonucleosides or ribonucleosides and 2′-O-Me modified nucleosides. A polynucleotide may comprise a mix of 2′-4′ bicyclic nucleosides and 2′-MOE, 2′-fluoro, or 2′-O-Me modified nucleosides. A polynucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt). A polynucleotide may comprise a mix of 2′-deoxyribonucleosides or ribonucleosides and 2′-fluoro modified nucleosides. A polynucleotide may comprise a mix of 2′-fluoro modified nucleosides and 2′-O-Me modified nucleosides.

In various embodiments, the oligonucleotide may comprise alternating nucleosides of different types. In certain embodiments, the oligonucleotide may comprise alternating deoxyribonucleosides or ribonucleosides and 2′-O-Me modified nucleosides. In certain embodiments, a polynucleotide may comprise alternating 2′-deoxyribonucleosides or ribonucleosides and 2′-fluoro modified nucleosides. In certain embodiments, the oligonucleotide may comprise alternating 2′-fluoro modified nucleosides and 2′-O-Me modified nucleosides. In certain embodiments, the oligonucleotide may comprise alternating 2′-4′ bicyclic nucleosides and 2′-MOE, 2′-fluoro, or 2′-O-Me modified nucleosides. In certain embodiments, the oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In various embodiments, a polynucleotide of the present disclosure may comprise one or more abasic residues, a 5-vinylphosphonate modification, and/or one or more inverted abasic residues.

In various embodiments, the oligonucleotide may comprise a phosphorothioate or other modified internucleoside linkage. In various embodiments, the oligonucleotide may comprise phosphorothioate internucleoside linkages. In various embodiments, the oligonucleotide comprises phosphorothioate internucleoside linkages between at least two nucleotides. In various embodiments, the oligonucleotide comprises phosphorothioate internucleoside linkages between all nucleotides. By way of a non-limiting example, in certain embodiments, oligonucleotides comprise modified internucleoside linkages at the first, second, and/or (e.g., and) third internucleoside linkage at the 5′ or 3′ end of the nucleotide sequence.

Non-limiting examples of phosphorus-containing linkages include aminoalkylphosphotriesters phosphorothioates, chiral phosphorothioates, phosphotriesters, phosphorodithioates, methyl and other alkyl phosphonates comprising 3′alkylene phosphonates and chiral phosphonates, phosphinates, phosphoramidates comprising 3′-amino phosphoramidate and aminoalkylphosphoramidates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 5,625,050; 4,469,863; 4,476,301; 5,023,243; 5,550,111; 5,177,196; 5,587,361; 5,188,897; 5,264,423; 5,276,019; 5,519,126; 5,278,302; 5,286,717; 5,321,131; 5,399,676; 5,405,939; 5,453,496; 5,455, 233; 5,466,677; 5,476,925; 5,536,821; 5,541,306; 5,563, 253; 5,571,799; and 3,687,808.

In various embodiments, a polynucleotide of the present disclosure may have heteroatom backbones, e.g., or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497), morpholino backbones (see Summerton and Weller, U.S. Pat. No. 5,034,506); amide backbones (see De Mesmaeker et al. Ace. Chem. Res. 1995, 28:366-374); or MMI or methylene (methylimino) backbones.

Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 6-O-methylguanine, 4-thio-pyrimidines, 4-amino-pyrimidines, 4-dimethylhydrazine-pyrimidines, and 4-O-alkyl-pyrimidines; U.S. Pat. No. 5,378,825 and PCT Publication No. WO 93/13121). For general discussion see Adams et al, The Biochemistry of the Nucleic Acids 5-36, 11^{th} ed., 1992. Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleotides and one or more nucleotide analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42): 13233-41). RNA and DNA have different sugar moieties and can differ by the presence of uracil or analogs thereof in RNA and thymine or analogs thereof in DNA.

Interfering Nucleic Acids

In some embodiments, a conjugated molecular cargo may comprise a polynucleotide molecule(s) which is capable of modifying expression of one more genes (e.g., inhibiting gene expression and/or translation, modulating RNA splicing or inducing exon skipping) in a target cell. In some embodiments, the polynucleotide molecule may be an interfering nucleic acid molecule, e.g., an siRNA, an shRNA, a miRNA, or an antisense oligonucleotide (ASO), that targets, e.g., an RNA (e.g., an mRNA).

In some embodiments, the interfering nucleic acid molecule may modify expression of one more genes associated with a skeletal muscle disease and/or disorder disclosed herein, for example, Double Homeobox 4 (DUX4), myotonic dystrophy protein kinase (DMPK, DMPK, also referred as DM, DM1, DM1PK, DMK, MDPK, MT-PK, Dm15, dystrophia myotonica protein kinase), dystrophin (DMD), F-Box Only Protein 32 (FBX032), Tripartite Motif Containing 63 (TRIM63), Inhibin Subunit Beta A (INHBA), Myostatin (MSTN), Myocyte Enhancer Factor 2D (MEF2D), KLF Transcription Factor 15 (KLF15), Mediator Complex Subunit 1 (MED1), Mediator Complex Subunit 13 (MED13), Protein Phosphatase 1 Regulatory Subunit 3A (PPP1R3A), Myosin Light Chain Kinase (MLCK1), and/or Activin A Receptor Type 1B (ACVR1B), Type-II SH2-domain-containing inositol 5-phosphatase (SHIP2).

In certain embodiments, interfering nucleic acid molecules that selectively target and inhibit the activity or expression of a product (e.g., an mRNA product) of a targeted gene are used in compositions and methods described herein. An interfering nucleic acid molecule may inhibit the expression or activity of a product (e.g., an mRNA product) of at least one targeted gene by at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, or 100%.

In some embodiments, an interfering nucleic acid molecule may inhibit the expression or activity of a product (e.g., an mRNA product) of at least one targeted gene by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more. In some embodiments, the expression or activity of a product (e.g., an mRNA product) of at least one targeted gene may be inhibited by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99%. In some embodiments, the expression or activity of a product (e.g., an mRNA product) of at least one targeted gene may be inhibited by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100%.

In some embodiments, an interfering nucleic acid molecule may inhibit the expression or activity of a product (e.g., an mRNA product) of at least one targeted gene by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, or at least 75%, or more.

In some embodiments, an interfering nucleic acid molecule may inhibit the expression or activity of a product (e.g., an mRNA product) of at least one targeted gene for at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, or at least 15 weeks, or more, e.g., following administration to a subject (i.e., post dosing). In some embodiments, an interfering nucleic acid molecule may inhibit the expression or activity of a product (e.g., an mRNA product) of at least one targeted gene for at least 3 weeks. In some embodiments, an interfering nucleic acid molecule may inhibit the expression or activity of a product (e.g., an mRNA product) for at least one targeted gene by at least 6 weeks.

In some embodiments, an interfering nucleic acid molecule may inhibit the expression or activity of a product (e.g., an mRNA product) of at least one targeted gene for at least about 1 month to at least about 2 months, for at least about 1 month to at least about 3 months, for at least about 1 month to at least about 4 months, for at least about 1 month to at least about 5 months, for at least about 1 month to at least about 6 months, for at least about 1 month to at least about 7 months, for at least about 1 month to at least about 8 months, for at least about 1 month to at least about 9 months, for at least about 1 month to at least about 10 months, for at least about 1 month to at least about 11 months, for at least about 1 month to at least about 12 months, for at least about 2 months to at least about 3 months, for at least about 2 months to at least about 4 months, for at least about 2 months to at least about 5 months, for at least about 2 months to at least about 6 months, for at least about 2 months to at least about 7 months, for at least about 2 months to at least about 8 months, for at least about 2 months to at least about 9 months, for at least about 2 months to at least about 10 months, for at least about 2 months to at least about 11 months, for at least about 2 months to at least about 12 months, for at least about 3 months to at least about 4 months, for at least about 3 months to at least about 5 months, for at least about 3 months to at least about 6 months, for at least about 3 months to at least about 7 months, for at least about 3 months to at least about 8 months, for at least about 3 months to at least about 9 months, for at least about 3 months to at least about 10 months, for at least about 3 months to at least about 11 months, for at least about 3 months to at least about 12 months, for at least about 4 months to at least about 5 months, for at least about 4 months to at least about 6 months, for at least about 4 months to at least about 7 months, for at least about 4 months to at least about 8 months, for at least about 4 months to at least about 9 months, for at least about 4 months to at least about 10 months, for at least about 4 months to at least about 11 months, for at least about 4 months to at least about 12 months, for at least about 5 months to at least about 6 months, for at least about 5 months to at least about 7 months, for at least about 5 months to at least about 8 months, for at least about 5 months to at least about 9 months, for at least about 5 months to at least about 10 months, for at least about 5 months to at least about 11 months, for at least about 5 months to at least about 12 months, for at least about 6 months to at least about 7 months, for at least about 6 months to at least about 8 months, for at least about 6 months to at least about 9 months, for at least about 6 months to at least about 10 months, for at least about 6 months to at least about 11 months, for at least about 6 months to at least about 12 months, for at least about 7 months to at least about 8 months, for at least about 7 months to at least about 9 months, for at least about 7 months to at least about 10 months, for at least about 7 months to at least about 11 months, for at least about 7 months to at least about 12 months, for at least about 8 months to at least about 9 months, for at least about 8 months to at least about 10 months, for at least about 8 months to at least about 11 months, for at least about 8 months to at least about 12 months, for at least about 9 months to at least about 10 months, for at least about 9 months to at least about 11 months, for at least about 9 months to at least about 12 months, for at least about 10 months to at least about 11 months, for at least about 10 months to at least about 12 months, for at least about 11 months to at least about 12 months, or more, e.g., following administration to a subject (i.e., post dosing).

In some embodiments, an interfering nucleic acid molecule may inhibit the expression or activity of a product (e.g., an mRNA product) of at least one targeted gene for at least 1 month, at least 2 months, at least 3 months, at least 4 months, at least 5 months, at least 6 months, at least 7 months, at least 8 months, at least 9 months, at least 10 months, at least 11 months, at least 12 months, or more, e.g., following administration to a subject (i.e., post dosing). In some embodiments, an interfering nucleic acid molecule may inhibit the expression or activity of a product (e.g., an mRNA product) of at least one targeted gene for at least 3 months. In some embodiments, an interfering nucleic acid molecule may inhibit the expression or activity of a product (e.g., an mRNA product) for at least one targeted gene by at least 6 months.

An agent disclosed herein may comprise a nucleobase sequence that is at least 80%, at least 85%, at least 90%, at least 95%, or 100% complementarity to a product (e.g., an mRNA product) of at least targeted gene. Without wishing to be bound by theory, “complementarity” of nucleic acids can mean that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

Interfering nucleic acids can include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence.

Typically, at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is single-stranded RNA. In some embodiments, the interfering nucleic acid molecule is double-stranded RNA. The double-stranded RNA molecule may have a 1-3 nucleotide 3′ and/or 5′ overhang in either a sense strand and/or an antisense strand. In some embodiments, the double-stranded RNA molecule has a 2 nucleotide 3′ overhang. In some embodiments, the two RNA strands are connected via a hairpin structure, forming a shRNA molecule. shRNA molecules can contain hairpins derived from microRNA molecules.

Interfering nucleic acid molecules described herein can contain RNA bases, non-RNA bases or a mixture of RNA bases and non-RNA bases. For example, interfering nucleic acid molecules described herein can be primarily composed of RNA bases or modified RNA bases, but also contain DNA bases, modified DNA bases, and/or non-naturally occurring nucleotides. The term “ribonucleotide” or “nucleotide” can, in the case of a modified RNA or nucleotide surrogate, also refer to a modified nucleotide, or surrogate replacement moiety at one or more positions.

In some embodiments, the interfering nucleic acid molecule is a small interfering RNAs (siRNA), also known as short interfering RNA or silencing RNA. siRNAs are a class of double-stranded RNA molecules, typically about 20-25 base pairs in length that target nucleic acids (e.g., mRNAs) for degradation via the RNA interference (RNAi) pathway in cells. Such siRNA molecules typically include a region of sufficient homology to the target region, and are of sufficient length in terms of nucleotides, such that the siRNA molecules down-regulate target nucleic acid. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

Specificity of siRNA molecules may be measured via the binding of the antisense strand of the molecule to its target RNA. Effective siRNA molecules are often fewer than 30 to 35 base pairs in length, e.g., to prevent stimulation of non-specific RNA interference pathways in the cell by way of the interferon response, however longer siRNA may also be effective. In various embodiments, the siRNA molecules are 5, 6, 7, 8, 9, 10, 11, 12, 13, 14, 15, 16, 17, 18, 19, 20, 21, 22, 23, 24, 25, 26, 27, 28, 29, or 30 base pairs in length. In various embodiments, the siRNA molecules are about 35 to about 70 more base pairs in length In some embodiments, the siRNA molecules are more than 70 base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, 21 to 24 base pairs in length. In some embodiments, the sense and antisense strands of the siRNA molecules are each independently about 19 to about 24 nucleotides in length. In some embodiments, the sense strand of an siRNA molecule is 23 nucleotides in length and the antisense strand is 21 nucleotides in length. In some embodiments, both the sense strand and the antisense strand of an siRNA molecule are 21 nucleotides in length.

After selection of a suitable target RNA sequence, siRNA molecules that comprise a nucleotide sequence complementary to all or a portion of the target sequence, i.e., an antisense sequence, may be designed and prepared using suitable methods (see, e.g., U.S. Patent Publication Nos. 2004/0077574 and 2008/0081791 and PCT Publication No. WO 2004/016735). In some embodiments, the siRNA molecule may be single-stranded (i.e. a ssRNA molecule comprising just an antisense strand) or double stranded (i.e. a dsRNA molecule comprising an antisense strand and a complementary sense strand that hybridizes to form the dsRNA). In various embodiments, the siRNA molecules may comprise a duplex, asymmetric duplex, hairpin or asymmetric hairpin secondary structure, comprising self-complementary sense and/or antisense strands.

In various embodiments, the antisense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiment, the antisense strand of the siRNA molecule is about 35 to about 70 nucleotides in length. In various embodiment, the antisense strand of the siRNA molecule is more than 70 nucleotides in length. In some embodiments, the antisense strand is 8 to 40 nucleotides in length, 10 to 20 nucleotides in length, 10 to 30 nucleotides in length, 15 to 20 nucleotides in length, 19 to 23 nucleotides in length, or 21 to 24 nucleotides in length.

In some embodiments, the sense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 more nucleotides in length. In various embodiments, the sense strand of the siRNA molecule is about 30 to about 70 nucleotides in length. In various embodiments, the sense strand of the siRNA molecule more than 70 nucleotides in length. In some embodiments, the sense strand is 8 to 40 nucleotides in length, 10 to 20 nucleotides in length, 10 to 30 nucleotides in length, 15 to 20 nucleotides in length, 19 to 23 nucleotides in length, 21 to 24 nucleotides in length.

In various embodiments, siRNA molecules can comprise an antisense strand comprising a region of complementarity to a target region in a target mRNA. In some embodiments, the region of complementarity is at least 70%, at least 75%, at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region may comprise a region of consecutive nucleotides in the target mRNA. In some embodiments, it may not be requisite for a region of complementarity to be 100% complementary to that of its target to be specifically hybridizable or specific for a target RNA sequence.

In some embodiments, siRNA molecules disclosed herein may comprise an antisense strand that comprises a region of complementarity to a target RNA sequence and the region of complementarity is in the range of 8 to 20, 8 to 35, 8 to 45, or 10 to 50, or 5 to 55, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of a target RNA sequence. In some embodiments, siRNA molecules comprise an antisense strand having a nucleotide sequence that contains no more than 1, 2, 3, 4, 5, 6, 7, 8, 9, or 10 base mismatches compared to the portion of the consecutive nucleotides of target RNA sequence. In some embodiments, siRNA molecules comprise a nucleotide sequence that has up to 3 mismatches over 15 bases, or up to 4 mismatches over 10 bases with a target sequence. In some embodiments, siRNA molecules comprises an antisense strand having a nucleotide sequence that has up 0, 1, 2, or 3 mismatches over 15-22 bases with a target sequence. In some embodiments, siRNA molecules comprises an antisense strand having a nucleotide sequence that has 0, 1, or 2 mismatches over 15-22 bases with a target sequence. In some embodiments, siRNA molecules comprises an antisense strand having a nucleotide sequence that has 0 or 1 mismatch over 15-22 bases with a target sequence. In some embodiments, siRNA molecules comprises an antisense strand having a nucleotide sequence that has 0 mismatches over 15-22 bases with a target sequence.

In various embodiments, siRNA molecules may comprise an antisense strand comprising a nucleotide sequence that is at least 70%, at least 75%, at least 85%, at least 90%, at least 95%, or 100% complementary to the target RNA sequence of the antisense oligonucleotides disclosed herein. In some embodiments, siRNA molecules comprise an antisense strand comprising a nucleotide sequence that is at least 70%, at least 75%, at least 85%, at least 90%, at least 95%, or 100% identical to any of the antisense oligonucleotides provided herein. In some embodiments, siRNA molecules comprise an antisense strand comprising at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of any of the antisense oligonucleotides provided herein.

In some embodiments, double-stranded siRNA can comprise sense and antisense RNA strands that are different lengths or the same length. In some embodiments, double-stranded siRNA molecules may also be generated from a single oligonucleotide in a stem-loop structure. The self-complementary sense and antisense regions of the siRNA molecule having a stem-loop structure may be linked by means of a nucleic acid based or a non-nucleic acid-based linker. In some embodiments, an siRNA having a stem-loop structure comprises a circular single-stranded RNA having two or more loop structures and a stem comprising self-complementary sense and antisense strands. In some embodiments, the circular RNA may be processed in vivo or in vitro to produce an active siRNA molecule which may be capable of mediating RNAi. Small hairpin RNA (shRNA) molecules are therefore also contemplated in the present disclosure. Such molecules may comprise a specific antisense sequence together with the reverse complement (sense) sequence, which may be separated by a spacer or loop sequence in some instances. A reverse complement described herein may comprise a sequence that is a complement sequence of a reference sequence, wherein the complement sequence is written in the reverse orientation. Due to codon usage redundancy, a reverse complement can diverge from a reference sequence that encodes the same polypeptide. As used herein, “reverse complement” also includes sequences that are, e.g., at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence. Cleavage of the spacer or loop can provide a single-stranded RNA molecule and its reverse complement, such that they may anneal to form a dsRNA molecule. In various embodiments, additional optional processing steps may result in removal or addition of 1, 2, 3, 4, 5 or more nucleotides from the 3′ end and/or the 5′ end of one or both strands. A spacer may be of a suitable length to allow the antisense and sense sequences to anneal and form a double-stranded structure or stem prior to cleavage of the spacer. In certain embodiments subsequent optional processing steps may result in removal or addition of 1, 2, 3, 4, 5 or more nucleotides from the 3′ end and/or the 5′ end of one or both strands. In some embodiments, a spacer sequence can be an unrelated nucleotide sequence that may be, e.g., situated between two complementary nucleotide sequence regions that, when annealed into a double-stranded nucleic acid, can comprise a shRNA.

The length of the siRNA molecules can vary from about 10 to about 120 nucleotides depending on the type of siRNA molecule being designed. Generally, between about 10 and about 55 of these nucleotides may be complementary to the RNA target sequence. For instance, when the siRNA is a double-stranded siRNA or single-stranded siRNA, the length can vary from about 10 to about 55 nucleotides, whereas when the siRNA is a shRNA or circular molecule, the length can vary from about 30 nucleotides to about 110 nucleotides.

In various embodiments, an siRNA molecule can comprise a 3′ overhang at one end of the molecule. In some embodiments, the other end can be blunt-ended or may also comprise an overhang (e.g., 5′ and/or 3′). When the siRNA molecule comprises an overhang at both ends of the molecule, the length of the overhangs may be different or the same. In some embodiments, an siRNA molecule described herein may comprises 3′ overhangs of about 1 to about 3 nucleotides on both ends of the molecule. In some embodiments, the siRNA molecule comprises 3′ overhangs of about 1 to about 3 nucleotides on both the sense strand and the antisense strand. In some embodiments, the siRNA molecule comprises 3′ overhangs of about 1 to about 3 nucleotides on the antisense strand. In some embodiments, the siRNA molecule may comprise 3′ overhangs of about 1 to about 3 nucleotides on the sense strand.

In various embodiments, the siRNA molecule comprises one or more modified nucleotides (e.g., 1, 2, 3, 4, 5, 6, 7, 8, 9, 10, 11, 12, 13, 14, 15 or more). In some embodiments, all of the nucleotides of the sense strand and/or the antisense strand of the siRNA molecule are modified. In certain embodiments, the siRNA molecule can comprise one or more modified nucleotides and/or one or more modified internucleotide linkages. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand.

In some embodiments, the modified nucleotide may comprise a modified sugar moiety (e.g., a 2′ modified nucleotide). In some embodiments, the siRNA molecule can comprise one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In various embodiments, each nucleotide of the siRNA molecule can a modified nucleotide (e.g., a 2′-modified nucleotide). In some embodiments, the siRNA molecule may comprise one or more phosphorodiamidate morpholinos. In some embodiments, each nucleotide of the siRNA molecule consists of a phosphorodiamidate morpholino.

In various embodiments, the siRNA molecule may comprise a phosphorothioate or other modified internucleotide linkage. In various embodiments, the siRNA molecule may comprise, e.g., a phosphorothioate internucleoside linkage(s). In some embodiments, the siRNA molecule may comprise a phosphorothioate internucleoside linkage(s) between two or more nucleotides. In some embodiments, the siRNA molecule may comprise a phosphorothioate internucleoside linkage(s) between all nucleotides. In some embodiments, the siRNA molecule may comprise modified internucleotide linkages at the first, second, and/or third internucleoside linkage at the 5′ or 3′ end of the siRNA molecule. In some embodiments, the siRNA molecule may comprise modified internucleotide linkages at the first and second internucleoside linkages at the 5′ and/or 3′ end of the siRNA molecule. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand. In some embodiments, the siRNA molecule may comprise modified internucleotide linkages at the first internucleoside linkage at the 5′ and 3′ ends of the siRNA molecule sense strand, at the first, second, and third internucleoside linkages at the 5′ end of the siRNA molecule antisense strand, and at the first internucleoside linkage at the 3′ end of the siRNA molecule antisense strand.

In various embodiments, the modified internucleotide linkages may comprise phosphorus-containing linkages. In some embodiments, phosphorus-containing linkages which may be used in the practice of the present disclosure include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3-5′ linkages, 2′-5′ linked analogs of these, and those having inverted polarity wherein the adjacent pairs of nucleoside units are linked 3-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the various modified formats or chemistries of siRNA molecules disclosed herein may be combined together. For example, without limitation, 1, 2, 3, 4, 5, 6, 7, 8, 9, or 10 or more different types of modifications may be included within the same siRNA molecule.

In various embodiments, the antisense strand may comprise one or more modified nucleotides (e.g., 1, 2, 3, 4, 5, 6, 7, 8, 9, 10, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, antisense strand contains a phosphorothioate or other modified internucleotide linkage. In some embodiments, the antisense strand may comprise phosphorothioate internucleoside linkage(s). In some embodiments, the antisense strand may comprise phosphorothioate internucleoside linkage(s) between two or more nucleotides. In some embodiments, the antisense strand may comprise phosphorothioate internucleoside linkage(s) between all nucleotides. In some embodiments, the antisense strand may comprise modified internucleotide linkages at the first, second, and/or third nucleotide at the 5′ or 3′ end of the antisense strand. In some embodiments, the antisense strand may comprise modified internucleotide linkages at the first and second nucleotide positions (e.g., between the first and second and between the second and third nucleotides) at the 5′ and 3′ ends of the antisense strand.

In various embodiments, the modified internucleotide linkages may comprise phosphorus-containing linkages of the antisense strand. In some embodiments, phosphorus-containing linkages which may be used in the practice of the present disclosure include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3-5′ linkages, 2′-5′ linked analogs of these, and those having inverted polarity wherein the adjacent pairs of nucleoside units are linked 3-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the modified formats or chemistries of the antisense strand disclosed herein may be combined together. For example, without limitation, 1, 2, 3, 4, 5, 6, 7, 8, 9, or 10 or more different types of modifications may be included within the same antisense strand.

In some embodiments, the sense strand comprises one or more modified nucleotides (e.g., 1, 2, 3, 4, 5, 6, 7, 8, 9, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, the sense strand contains a phosphorothioate or other modified internucleotide linkage. In some embodiments, the sense strand may comprise phosphorothioate internucleoside linkage(s). In some embodiments, the sense strand may comprise phosphorothioate internucleoside linkage(s) between two or more nucleotides. In some embodiments, the sense strand may comprise phosphorothioate internucleoside linkages between all nucleotides. For example, in some embodiments, the sense strand comprises modified internucleotide linkages at the first, second, and/or third nucleotide at the 5′ or 3′ end of the sense strand. In some embodiments, the sense strand may comprise modified internucleotide linkages at the first and second nucleotide positions (e.g., between the first and second and between the second and third nucleotides) at the 5′ end of the sense strand.

In various embodiments, the modified internucleotide linkages may comprise phosphorus-containing linkages of the sense strand. In some embodiments, phosphorus-containing linkages which may be used in the practice of the present disclosure include, without limitation, chiral phosphorothioates, phosphorothioates, phosphorodithioates, aminoalkylphosphotriesters, phosphotriesters, methyl and other alkyl phosphonates comprising 3′alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the modified chemistries or formats of the sense strand described herein can be combined together. For example, without limitation, 1, 2, 3, 4, 5, 6, 7, 8, 9, or 10 or more different types of modifications may be included within the same sense strand.

In various embodiments, the antisense and/or sense strand of the siRNA molecule may comprise one or more modifications capable of enhancing or reducing, e.g., RNA-induced silencing complex (RISC) loading. In some embodiments, the antisense strand of the siRNA molecule may comprise one or more modifications capable of enhancing RISC loading. In various embodiments, the sense strand of the siRNA molecule may comprise one or more modifications capable of reducing RISC loading and/or reducing off-target effects. In various embodiments, the antisense strand of the siRNA molecule may comprise a 2′-O-methoxyethyl (2′-MOE) modification. In some embodiments, the addition of the 2′-O-methoxyethyl (2′-MOE) group, e.g., at the cleavage site may improve the silencing activity and/or specificity of siRNAs, e.g., by facilitating the oriented RNA-induced silencing complex (RISC) loading of the modified strand, e.g., as disclosed in Song et al., (2017) Mol Ther Nucleic Acids 9:242-250, incorporated herein by reference in its entirety. In various embodiments, the antisense strand of the siRNA molecule may comprise a 2′-O-Me-phosphorodithioate modification. In some embodiment, the 2′-O-Me-phosphorodithioate modification may increase RISC loading, e.g., as disclosed in Wu et al., (2014) Nat Commun 5:3459, incorporated herein by reference in its entirety.

In various embodiments, the sense strand of the siRNA molecule may comprise a 5′-nitroindole modification. In some embodiments, the 5′-nitroindole modification may decrease the RNAi potency of the sense strand and/or reduces off-target effects, e.g., as disclosed in Zhang et al., (2012) Chembiochem 13(13): 1940-1945, incorporated herein by reference in its entirety. In various embodiments, the sense strand may comprise a 2′-O-methyl (2′-O-Me) modification. In some embodiments, the 2′-O-Me modification may reduce RISC loading and/or the off-target effects of the sense strand, e.g., as disclosed in Zheng et al., FASEB (2013) 27(10): 4017-4026, incorporated herein by reference in its entirety. In various embodiments, the sense strand of the siRNA molecule may be fully substituted with morpholino, 2′-MOE and/or 2′-O-Me residues, and may not be recognized by RISC, e.g., as disclosed in Kole et al., (2012) Nature reviews. Drug Discovery 11(2): 125-140, incorporated herein by reference in its entirety.

In various embodiments, the sense strand of the siRNA molecule may comprise a 5′-morpholino modification. In various embodiments, the 5′-morpholino modification may reduce RISC loading of the sense strand and/or improves RNAi activity and/or antisense strand selection, e.g., as disclosed in Kumar et al., (2019) Chem Commun (Camb) 55(35):5139-5142, incorporated herein by reference in its entirety. In various embodiments, the sense strand of the siRNA molecule may be modified, for example, with a synthetic RNA-like high affinity nucleotide analogue called Locked Nucleic Acid (LNA) that may reduce RISC loading of the sense strand and promote antisense strand incorporation into RISC, e.g., as disclosed in Elman et al., (2005) Nucleic Acids Res. 33(1): 439-447, incorporated herein by reference in its entirety. In various embodiments, the sense strand of the siRNA molecule may comprise a 5′ unlocked nucleic acid (UNA) modification. In various embodiments, the 5′ unlocked nucleic acid (UNA) modification may reduce RISC loading of the sense strand and/or improve silencing capability of the antisense strand, e.g., as disclosed in Snead et al., (2013) Mol Ther Nucleic Acids 2(7):e103, incorporated herein by reference in its entirety.

In some embodiments, the antisense strand of the siRNA molecule may comprise a 2′-MOE modification and/or the sense strand may comprise an 2′-O-Me modification (see e.g., Song et al., (2017) Mol Ther Nucleic Acids 9:242-250). In some embodiments at least one (e.g., at least 2, at least 3, at least 4, at least 5, at least 6, at least 5, at least 8, at least 9, at least 10 or more) siRNA molecule may be conjugated, for example, covalently to a CACNG1-binding protein described herein. In some embodiments, the CACNG1-binding protein may be conjugated to the 5′ end of the sense strand of the siRNA molecule. In some embodiments, the CACNG1-binding protein may be conjugated to the 3′ end of the sense strand of the siRNA molecule. In some embodiments, the CACNG1-binding protein may be conjugated internally to the sense strand of the siRNA molecule. In some embodiments, the CACNG1-binding protein may be conjugated to the 5′ end of the antisense strand of the siRNA molecule. In some embodiments, the CACNG1-binding protein may be conjugated to the 3′ end of the antisense strand of the siRNA molecule. In some embodiments, the CACNG1-binding protein be conjugated internally to the antisense strand of the siRNA molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-termini of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (e.g., C3-C12 (e.g., C3, C6, C9, C12), abasic, tri ethylene glycol, hexaethylene glycol), biotin or fluorescein reagents that come as phosphoramidites and that have another DMT-protected hydroxyl group, allowing multiple couplings during RNA synthesis.

In some embodiments, the sense strand is 23 nucleotides in length and the antisense strand is 21 nucleotides in length. In some embodiments, the sense strand is 23 nucleotides in length and the antisense strand is 21 nucleotides in length, wherein the 3′ and 5′ terminal nucleotide positions of the sense strand are inverted abasic residues. The sense strand 3′ and 5′ terminal inverted abasic residues may be overhangs. The inverted abasic residues may be linked via a 3′-3′ phosphodiester linkage. In some embodiments, the antisense strand of the siRNA molecule contains 1-2 phosphorothioate linkages at the 3′ and/or 5′ ends. In some embodiments, the antisense strand contain two or three phosphorothioate internucleotide linkages at the 5′-terminus and 1 phosphorothioate internucleotide linkage at the 3′-terminus. The siRNA molecule may be linked to a targeting moiety at the 5′ or 3′ end of the sense strand.

In some embodiments, the sense strand is 21 nucleotides in length and the antisense strand is 23 nucleotides in length, wherein the antisense strand contains a 2 nucleobase 3′ overhang. In some embodiments, the antisense strand of the siRNA molecule contains 1-3 phosphorothioate linkages at the 3′ and 5′ ends and the sense strand of the siRNA molecule contains 1-2 phosphorothioate linkages at the 5′ end. In some embodiments, the antisense strand of the siRNA molecule contains 2-3 phosphorothioate linkages at the 5′ end and 2 phosphorothioate linkages at the 3′, and the sense strand of the siRNA molecule contains 2 phosphorothioate linkages at the 5′ end. The siRNA molecule may be linked to a targeting moiety at the 5′ or 3′ end of the sense strand.

In some embodiments, the interfering nucleic acid molecule is a short hairpin RNA (shRNA). A “small hairpin RNA” or “short hairpin RNA” or “shRNA” described herein may include a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure may be cleaved by the cellular machinery into siRNA, which is then bound to the RNA-induced silencing complex (RISC).

Non-limiting examples of shRNAs include a double-stranded polynucleotide molecule assembled from a single-stranded molecule, where the sense and antisense regions are linked by a nucleic acid-based or non-nucleic acid-based linker, and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. Patent Publication No. 2011/0071208, the disclosure of which is herein incorporated by reference in its entirety for all purposes.

In some embodiments, the interfering nucleic acid molecule is a microRNA (miRNA). miRNAs represent a large group of small RNAs produced naturally in organisms, some of which regulate the expression of target genes. miRNAs are short hairpin RNAs about 18 to about 25 nucleotides in length that function in RNA silencing and post-translational regulation of gene expression. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer. miRNAs are not translated into proteins, but instead bind to specific messenger RNAs, thereby blocking translation. In some embodiments, miRNAs base-pair imprecisely with their targets to inhibit translation.

miRNAs as described herein can include pri-miRNA, pre-miRNA, mature miRNA or fragments of variants thereof that retain the biological activity of mature miRNA. In some embodiments, the size range of the miRNA can be from 21 nucleotides to 170 nucleotides. In one embodiment, the size range of the miRNA is from 70 to 170 nucleotides in length. In another embodiment, mature miRNAs of from 21 to 25 nucleotides in length can be used.

In certain embodiments, the interfering nucleic acid molecule is an antisense oligonucleotide (ASO). An ASO can down regulate a target by inducing RNase H endonuclease cleavage of a target RNA, by steric hindrance of ribosomal activity, by inhibiting 5′ cap formation, or by altering splicing. An ASO can be, but is not limited to, a gapmer or a morpholino. An antisense oligonucleotide typically comprises a short nucleotide sequence which is substantially complementary to a target nucleotide sequence in a pre-mRNA molecule, heterogeneous nuclear RNA (hnRNA) or mRNA molecule. The degree of complementarity (or substantial complementarity) of the antisense sequence is preferably such that a molecule comprising the antisense sequence can form a stable double stranded hybrid with the target nucleotide sequence in the RNA molecule under physiological conditions. Antisense oligonucleotides are often synthetic and chemically modified.

Antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

In some embodiments, an interfering nucleic acid molecule described herein is a gapmer. A “Gapmer” is oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” A gapmer can have 5′ and 3′ wings each having 2-6 nucleotides and a gap having 7-12 nucleotides. In some embodiments, a gapmer can have a 3-10⁻³ configuration or a 5-10⁻⁵ configuration.

A gapmer commonly has the formula 5′-X—Y—Z-3′, with X and Z as flanking regions around a gap region Y. In some embodiments, flanking region X of formula 5′-X—Y—Z-3′ is also called X region, flanking sequence X, 5′ wing region X, or 5′ wing segment. In some embodiments, flanking region Z of formula 5′-X—Y—Z-3′ is also called Z region, flanking sequence Z, 3′ wing region Z, or 3′ wing segment. In some embodiments, gap region Y of formula 5′-X—Y—Z-3′ is also called Y region, Y segment, gap-segment Y, gap segment, or gap region. In some embodiments, each nucleoside in the gap region Y is a 2′-deoxyribonucleoside, and neither the 5′ wing region X or the 3′ wing region Z comprises any 2′-deoxyribonucleosides.

In some embodiments, the gap region of the gapmer polynucleotide may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, or at least five or more nucleotides. In some embodiments, each internucleotide linkage in the gap segment comprises a phosphorothioate linkage. In some embodiments, the gap region and two flanking regions each independently comprise modified internucleoside linkages (e.g., phosphorothioate internucleoside linkages or other linkages) between at least two, at least three, at least four, or at least five or more nucleotides. In some embodiments, each internucleotide linkage in the 5′ or 3′ wing region comprises a phosphorothioate linkage. In some embodiments, each internucleotide linkage in the gapmer comprises a phosphorothioate linkage.

In some embodiments, the Y region may comprise a contiguous stretch of nucleotides, e.g., a region of 5 or more DNA nucleotides, which can be capable of recruiting an RNase including but not limited to RNase H. In some embodiments, the gapmer may bind to a target nucleic acid such that an RNase is recruited to cleave the target nucleic acid. In some embodiments, the Y region may be flanked both 5′ and 3′ by regions X and Z comprising high-affinity modified nucleosides, e.g., 1-10 high-affinity modified nucleosides. Exemplary high affinity modified nucleosides include, without limitation, 2′-4′ bicyclic nucleosides (e.g., LNA, cEt, ENA) and 2′-modified nucleosides (e.g., 2′-MOE, 2′0-Me, 2′-F). In some embodiments, the flanking sequences X and Z may be of 1-30 nucleotides, 1-20 nucleotides, 1-10 nucleotides, or 1-5 nucleotides in length. The flanking sequences X and Z may be of similar length or of dissimilar lengths. In some embodiments, the flanking sequences X and Z are each 5 nucleotides in length. In some embodiments, the flanking sequences X and Z are each 3 nucleotides in length. In some embodiments, the gap-segment Y may be a nucleotide sequence of 5-30 nucleotides, 5-20 nucleotides, or 5-10 nucleotides in length. In some embodiments, the gap segment is 10 nucleotides in length.

A gapmer may be produced using suitable methods. Preparation of gapmers is described in, for example, U.S. Pat. Nos. 10,260,069; 10,017,764; 9,695,418; 9,428,534; 9,428,534; 9,045,754; 8,580,756; 8,580,756; 7,750,131; 7,683,036; 7,569,686; 7,432,250; 7,399,845; 7,101,993; 7,015,315; 5,898,031; 5,700,922; 5,652,356; 5,652,355; 5,623,065; 5,565,350; 5,491,133; 5,403,711; 5,366,878; 5,256,775; 5,220,007; 5,149,797; and 5,013,830; U.S. Patent Publication Nos. US2010/0197762, US2005/0074801, US2009/0221685, US2009/0286969, and US2011/0112170; PCT Publication Nos. WO2005/023825, WO2004/069991, WO2008/049085 and WO2009/090182, each of which is herein incorporated by reference in its entirety.

In some embodiments, a gapmer is 10⁻⁵⁰ nucleosides in length. For example, a gapmer may be 10-50, 10-45, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length. In some embodiments, a gapmer is about 16 to about 20 nucleosides in length. In some embodiments, a gapmer is 16 nucleotides in length. In some embodiments, a gapmer is 20 nucleotides in length.

In some embodiments, the 5 wing region and the 3′ wing region of a gapmer are independently 1-20 nucleosides (e.g., 1, 2, 3, 4, 5, 6, 7, 8, 9, 10, 11, 12, 13, 14, 15, 16, 17, 18, 19, or 20 nucleosides) long. For example, the Swing region and the 3′ wing region of the gapmer may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′ wing region and the 3′ wing region of the gapmer are of the same length. In some embodiments, the 5′ wing region and the 3′ wing region of a gapmer are of different lengths. In some embodiments, the 5′ wing region is longer than the 3′ wing region of a gapmer. In some embodiments, the 5′ wing region is shorter than the 3′ wing region of the gapmer.

In some embodiments, the gap region in a gapmer is 5-20 nucleosides in length. For example, the gap region Y may be 5-20, 5-15, 5-10, 10-20, 10-15, or 15-20 nucleosides in length. In some embodiments, the gap region is 5, 6, 7, 8, 9, 10, 11, 12, 13, 14, 15, 16, 17, 18, 19, or 20 nucleosides in length. In some embodiments, one or more nucleosides in the gap region Y is a 2′-deoxyribonucleoside. In some embodiments, every nucleotide in the gap region is a deoxyribonucleoside. In some embodiments, one or more of the nucleosides in the gap region is a modified nucleoside (e.g., a 2′ modified nucleoside such as those described herein). In some embodiments, one or more cytosines in the gap region Y are 5-methyl-cytosines. In some embodiments, every cytosine in the gap region Y is a 5-methyl-cytosine. In some embodiments, every cytosine in a gapmer is a 5-methyl-cytosine.

In some embodiments, one or more nucleosides in the 5 wing region or the 3′ wing region of a gapmer are modified nucleotides. In some embodiments, the modified nucleotide may be a 2′-modified nucleoside, e.g., 2′-4′ bicyclic nucleoside or a non-bicyclic 2′-modified nucleoside. In some embodiments, the nucleoside may be a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-dimethylaminoethyl (2′-O-DMAOE), 2′-O-dimethylaminopropyl (2′-O-DMAP), 2′-O-methoxyethyl (2′-MOE), 2′-O-aminopropyl (2′-O-AP), 2′-O-dimethylaminoethyloxyethyl (2′-O-DMAEOE), or 2′-O—N-methylacetamido (2′-O—NMA)). In some embodiments, every nucleotide in a wing region is a modified nucleotide. In some embodiments, every nucleotide in a wing region is a 2′-MOE, LNA or cET nucleotide.

In some embodiments, a gapmer of the present disclosure may comprises one or more modified nucleoside linkages in each of the X, Y, and Z regions. In some embodiments, each internucleoside linkage may comprise phosphorothioate linkage. In some embodiments, each of the X, Y, and Z regions independently comprises a combination of phosphodiester linkages and phosphorothioate linkages. In some embodiments, each internucleoside linkage in the gap region Y may be a phosphorothioate linkage, the 5′ wing region X comprises a combination of phosphorothioate linkages and phosphodiester linkages, and the 3′ wing region Z comprises a combination of phosphorothioate linkages and phosphodiester linkages.

In some embodiments, each nucleotide in the gap region of a gapmer is a deoxyribonucleotide and each nucleotide in a wing region is a 2′-MOE nucleotide. In some embodiments, each nucleotide in the gap region of a gapmer is a deoxyribonucleotide, each nucleotide in a wing region is a 2′-MOE nucleotide, and every cytosine in the gapmer is a 5-methyl-cytosine. In some embodiments, each nucleotide in the gap region of a gapmer is a deoxyribonucleotide, each nucleotide in a wing region is a 2′-MOE nucleotide, every cytosine in the gapmer is a 5-methyl-cytosine and every internucleotide linkage is a phosphorothioate linkage.

In some embodiments, each nucleotide in the gap region of a gapmer is a deoxyribonucleotide and each nucleotide in a wing region is a LNA nucleotide. In some embodiments, each nucleotide in the gap region of a gapmer is a deoxyribonucleotide, each nucleotide in a wing region is a LNA nucleotide, and every cytosine in the gapmer is a 5-methyl-cytosine. In some embodiments, each nucleotide in the gap region of a gapmer is a deoxyribonucleotide, each nucleotide in a wing region is a LNA nucleotide, every cytosine in the gapmer is a 5-methyl-cytosine and every internucleotide linkage is a phosphorothioate linkage. In some embodiments, each nucleotide in the gap region of a gapmer is a deoxyribonucleotide and each nucleotide in a wing region is a cET nucleotide. In some embodiments, each nucleotide in the gap region of a gapmer is a deoxyribonucleotide, each nucleotide in a wing region is a cET nucleotide, and every cytosine in the gapmer is a 5-methyl-cytosine. In some embodiments, each nucleotide in the gap region of a gapmer is a deoxyribonucleotide, each nucleotide in a wing region is a cET nucleotide, every cytosine in the gapmer is a 5-methyl-cytosine and every internucleotide linkage is a phosphorothioate linkage.

The interfering nucleic acids can employ a variety of oligonucleotide chemistries. Examples of oligonucleotide chemistries include, without limitation, peptide nucleic acid (PNA), locked nucleic acid (LNA), phosphorothioate, 2′-O-Me-modified oligonucleotides, and morpholino chemistries, including combinations of any of the foregoing. In general, PNA and LNA chemistries can utilize shorter targeting sequences because of their relatively high target binding strength relative to 2′-O-Me oligonucleotides. Phosphorothioate and 2′-O-Me-modified chemistries are often combined to generate 2′-O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications. The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 5,539,082; 5,714,331; and 5,719,262, 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids described herein may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties. Alternatively, non-phosphorous containing linkers may be employed. In some embodiments, an antisense oligonucleotides comprises an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases SI and PI, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., lyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′ 0-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′-O-Me oligonucleotides (phosphodiester or phosphothioate) can be synthesized according to routine techniques in the art (see, e.g., Yoo et al., Nucleic Acids Res. 32:2008-16, 2004).

Interfering nucleic acid molecules can be prepared, for example, by chemical synthesis, in vitro transcription, or digestion of long dsRNA by RNase III or Dicer. These can be introduced into cells by transfection, electroporation, or other methods known in the art. See Hannon, G J, 2002, Nature 418: 244-251; Bernstein E et al., 2002, RNA 7: 1509-1521; Hutvagner G et al., Curr. Opin. Genetics & Development 12: 225-232; Brummelkamp, 2002, Science 296: 550-553; Lee N S, et al. 2002. Nature Biotechnol. 20:500-505; Miyagishi M, and Taira K. 2002. Nature Biotechnol. 20:497-500; Paddison P J, et al., 2002. Genes & Dev. 16:948-958; Paul C P, et al., 2002. Nature Biotechnol. 20:505-508; Sui G et al., 2002. Proc. Natl. Acad. Sci. USA 99(6):5515-5520; Yu J-Y et al., 2002. Proc. Natl. Acad. Sci. USA 99(9):6047-6052. Each of the foregoing is incorporated by reference in its entirety.

Guide RNAs

In some embodiments, a conjugated molecular cargo comprises a guide RNA or a DNA encoding a guide RNA. A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 366) or GUUUUAGAGCUAUGCUGUUUUG (SEQ ID NO: 367). Any of the DNA-targeting segments disclosed herein can be joined to the 5′ end of SEQ ID NO: 366 or 367 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of AGCAUAGCAAGUUAAAAUAAGGCUAGUCCGUUAUCAACUUGAAAAAGUGGCACCGAG UCGGUGCUUU (SEQ ID NO: 368), AAACAGCAUAGCAAGUUAAAAUAAGGCUAGUCCGUUAUCAACUUGAAAAAGUGGCAC CGAGUCGGUGCUUUU (SEQ ID NO: 369), or GUUGGAACCAUUCAAAACAGCAUAGCAAGUUAAAAUAAGGCUAGUCCGUUAUCAACU UGAAAAAGUGGCACCGAGUCGGUGC (SEQ ID NO: 370).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

(version 1; SEQ ID NO: 371)
GUUUUAGAGCUAGAAAUAGCAAGUUAAAAUAAGGCUAGUCCGUUAUCAA
CUUGAAAAAGUGGCACCGAGUCGGUGCU;
(version 2; SEQ ID NO: 372)
GUUGGAACCAUUCAAAACAGCAUAGCAAGUUAAAAUAAGGCUAGUCCGU
UAUCAACUUGAAAAAGUGGCACCGAGUCGGUGC;
(version 3; SEQ ID NO: 373)
GUUUUAGAGCUAGAAAUAGCAAGUUAAAAUAAGGCUAGUCCGUUAUCAA
CUUGAAAAAGUGGCACCGAGUCGGUGC;
and
(version 4; SEQ ID NO: 374)
GUUUAAGAGCUAUGCUGGAAACAGCAUAGCAAGUUUAAAUAAGGCUAGU
CCGUUAUCAACUUGAAAAAGUGGCACCGAGUCGGUGC;
(version 5; SEQ ID NO: 375)
GUUUUAGAGCUAGAAAUAGCAAGUUAAAAUAAGGCUAGUCCGUUAUCAA
CUUGAAAAAGUGGCACCGAGUCGGUGCUUUUUUU;
(version 6; SEQ ID NO: 376)
GUUUUAGAGCUAGAAAUAGCAAGUUAAAAUAAGGCUAGUCCGUUAUCAA
CUUGAAAAAGUGGCACCGAGUCGGUGCUUUU;
(version 7; SEQ ID NO: 377)
GUUUAAGAGCUAUGCUGGAAACAGCAUAGCAAGUUUAAAUAAGGCUAGU
CCGUUAUCAACUUGAAAAAGUGGCACCGAGUCGGUGCUUUUUU;
or
(version 8; SEQ ID NO: 378)
GUUUUAGAGCUAGAAAUAGCAAGUUAAAAUAAGGCUAGUCCGUUAUCAA
CUUGGCACCGAGUCGGUGC.

In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

In some cases, a guide RNA for use in a transcriptional activation system comprising a dCas9-VP64 fusion protein paired with MS2-p65-HSF1 can be used. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence:

(SEQ ID NO: 379)
mN*mN*mN*NNNNNNNNNNNNNNNNNGUUUUAGAmGmCmUmAmGmAmAm
AmUmAmGmCAAGUUAAAAUAAGGCUAGUCCGUUAUCAmAmCmUmUmGmA
mAmAmAmAmGmUmGmGmCmAmCmCmGmAmGmUmCmGmGmUmGmCmU*mU
*mU*mU,

where “N” may be any natural or non-natural nucleotide. The totality of N residues can comprise a DNA-targeting segment as described herein. The terms “mA,” “mC,” “mU,” and “mG” denote a nucleotide (A, C, U, and G, respectively) that has been modified with 2′-O-Me. The symbol “*” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be conjugated to the CACNG1-binding protein disclosed herein, such as an scFv or an antibody or an antigen-binding fragment thereof, in the form of RNA, either as two molecules (separate crRNA and tracrRNA) or as one molecule (sgRNA), and optionally in the form of a complex with a Cas protein.

The gRNA can be conjugated to the CACNG1-binding protein disclosed herein, such as an scFv or an antibody or an antigen-binding fragment thereof, in the form of DNA encoding the gRNA. The DNA encoding the gRNA can encode a single RNA molecule (sgRNA) or separate RNA molecules (e.g, separate crRNA and tracrRNA). In the latter case, the DNA encoding the gRNA can be provided as one DNA molecule or as separate DNA molecules encoding the crRNA and tracrRNA, respectively.

Multiple gRNAs can be conjugated to the CACNG1-binding protein disclosed herein, such as an scFv or an antibody or an antigen-binding fragment thereof. The gRNAs can be the same or different gRNAs, or can target the same gene or different genes. In some embodiments, 1, 2, 3, 4, 5 or more guide RNAs are conjugated to the CACNG1-binding protein disclosed herein, such as an scFv or an antibody or an antigen-binding fragment thereof.

Alternatively, the gRNA, either in the form of RNA or DNA, may be incorporated into a carrier (e.g., liposomes or LNPs) which is conjugated to the CACNG1-binding protein disclosed herein, such as an scFv or an antibody or an antigen-binding fragment thereof. The carrier can further comprise a Cas protein, such as a Cas9 protein, or a nucleic acid (e.g., mRNA) encoding a Cas protein. Carriers such as liposomes or lipid nanoparticles are described in further detail below.

Multiple gRNAs can be incorporated into a carrier (e.g., liposome or LNP) which is conjugated to the CACNG1-binding protein disclosed herein, such as an scFv or an antibody or an antigen-binding fragment thereof. The gRNAs can be the same or different gRNAs, or can target the same gene or different genes. In some embodiments, 1, 2, 3, 4, 5 or more guide RNAs are incorporated into a carrier (e.g., liposome or LNP) which is conjugated to the CACNG1-binding protein disclosed herein, such as an scFv or an antibody or an antigen-binding fragment thereof.

When a gRNA is provided in the form of DNA, the gRNA after being delivered to the target cell can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand”.

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

The target sequence (e.g., guide RNA target sequence) for the DNA-binding protein can be anywhere within a targeted gene that is suitable for altering expression of the targeted gene. As one example, the target sequence can be within a regulatory element, such as an enhancer or promoter, or can be in proximity to a regulatory element. For example, the target sequence can be within about 10, 20, 30, 40, 50, 100, 200, 300, 400, 500, or 1,000 nucleotides of the start codon.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-NiGG-3′, where Ni is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN2-3′, where N2 is any DNA nucleotide and is immediately 5′ of the sequence to which the DNA-targeting segment of the guide RNA hybridizes on the complementary strand of the target DNA. In some such cases, Ni and N2 can be complementary and the Ni—N2 base pair can be any base pair (e.g, Ni═C and N2=G; Ni=G and N2=C; Ni=A and N2=T; or Ni=T, and N2=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. The guanine at the 5′ end can facilitate transcription by RNA polymerase in cells. Other examples of guide RNA target sequences plus PAMs can include two guanine nucleotides at the 5′ end to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length, including the 5′ G or GG and the 3′ GG or NGG. Yet other guide RNA target sequences plus PAMs can have between 14 and 20 nucleotides in length.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

Other Types of Polynucleotide Molecules

In some embodiments, a molecular cargo, e.g., a polynucleotide molecule described herein may comprise a ribozyme (ribonucleic acid enzyme). Without wishing to be bound by theory, a ribozyme is a molecule, commonly an RNA molecule, that is capable of performing specific biochemical reactions, akin to the action of protein enzymes. Ribozymes comprise molecules possessing catalytic activities such as, but not limited to, the capacity to cleave at specific phosphodiester linkages in RNA molecules to which they have hybridized, e.g., RNA-containing substrates, lncRNAs, mRNAs, and ribozymes.

Ribozymes may take on one of several physical structures, one such structure is termed “hammerhead”. A hammerhead ribozyme can comprise, e.g., a catalytic core comprising nine conserved bases, two regions complementary to the target RNA flanking regions the catalytic core, and a double-stranded stem and loop structure (stem-loop II). The flanking regions may permit the binding of the ribozyme to the target RNA, in particular, by forming double-stranded stems I and III. Cleavage may occur in trans (cleavage of an RNA substrate other than that containing the ribozyme) or in cis (cleavage of the same RNA molecule that contains the hammerhead motif) adjacent to a specific ribonucleotide triplet by a transesterification reaction from a 3′, 5′-phosphate diester to a 2′, 3-cyclic phosphate diester. In certain embodiments, this catalytic activity may require the presence of specific, highly conserved sequences in the catalytic region of the ribozyme.

Modifications in ribozyme structure can include the replacement or substitution of non-core portions of the molecule with non-nucleotidic molecules. As a non-limiting example, Ma et al. (Biochem. (1993) 32:1751-1758; Nucleic Acids Res. (1993) 21:2585-2589) replaced the six-nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length. Benseler et al. (J. Am. Chem. Soc. (1993) 115:8483-8484) describes hammerhead-like molecules where two of the base pairs of stem II, and all four of the nucleotides of loop II may be replaced with non-nucleoside linkers based on bis(propanediol) phosphate, hexaethylene glycol, bis(triethylene glycol) phosphate, propanediol, or tris(propanediol)bisphosphate.

Ribozyme polynucleotides may be generated using any of various suitable methods known in the art (see, e.g., U.S. Pat. Nos. 5,436,143 and 5,650,502; and PCT Publications Nos. WO94/13688; WO91/18624, WO92/01806; and WO 92/07065) or can be obtained from commercial sources (e.g., US Biochemicals), the contents of each of which are incorporated herein by reference in their entirety. In some embodiments, the ribozyme polynucleotide described herein can incorporate nucleotide analogs, e.g., to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme RNA sequences maybe synthesized conventionally, for example, by using RNA polymerases such as T7 or SP6. The ribozyme may also be produced in recombinant vectors by suitable means.

In some embodiments, internucleotidic phosphorus atoms of the polynucleotide molecules disclosed herein may be chiral, and the properties of the polynucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43, the contents of which are incorporated herein by reference in their entirety). In some embodiments, phosphorothioate-containing oligonucleotides may comprise nucleoside units that can be joined together by either substantially all Rp or substantially all Sp phosphorothioate inter-sugar linkages. In some embodiments, such phosphorothioate oligonucleotides comprising substantially chirally pure inter-sugar linkages may be produced via chemical synthesis or enzymatic approaches, as disclosed, e.g., in U.S. Pat. No. 5,587,261, the contents of which are incorporated herein by reference in their entirety. In some embodiments, chirally controlled polynucleotide molecules described may provide selective cleavage patterns of a target nucleic acid. As a non-limiting example, a chirally controlled polynucleotide molecule may provide single site cleavage within a complementary sequence of a nucleic acid, as disclosed, for example, in US Patent Publication No. 2017/0037399, the contents of which are incorporated herein by reference in their entirety.

In some embodiments, the polynucleotide molecule described herein may be a morpholino-based compound. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties). Morpholino-based oligomeric compounds are also described in, e.g., U.S. Pat. No. 5,034,506, and Genesis, volume 30, issue 3, 2001; Heasman, J., Dev. Biol., 2002, 243, 209-214; Dwaine A. Braasch and David R. Corey, Biochemistry, 2002, 41(14), 4503-4510); Nasevicius et al., Nat. Genet., 2000, 26, 216-220; Lacerra et al., Proc. Natl. Acad. Sci., 2000, 97, 9591-9596, the disclosures of which are incorporated herein by reference in their entireties.

In some embodiments, a polynucleotide molecule described herein may comprise an aptamer. An aptamer may comprise any nucleic acid which specifically binds specifically to a target, e.g., protein or nucleic acid in a cell. In some embodiments, the aptamer is a DNA aptamer or an RNA aptamer. In some embodiments, a nucleic acid aptamer may comprise a single-stranded RNA (ssDNA or ssRNA) or DNA. In certain embodiments, a single-stranded nucleic acid aptamer may form loop(s) and/or helice(s) structures. The nucleic acid that forms the nucleic acid aptamer may comprise naturally occurring nucleotides, modified nucleotides with hydrocarbon or PEG linkers inserted between one or more nucleotides, modified nucleotides, naturally occurring nucleotides with hydrocarbon linkers (e.g., an alkylene) or a polyether linker (e.g., a PEG linker) inserted between one or more nucleotides, or a combination of thereof. Aptamers and method of producing aptamers are described in, e.g., U.S. Pat. Nos. 8,318,438, 5,650,275; 5,683,867; 5,670,637; 5,696,249; 5,789,157; 5,843,653; 5,270,163; 5,567,588, 5,864,026; 5,989,823; 6,569,630; and PCT Publication No. WO 99/31275, Lorsch and Szostak, 1996; Jayasena, 1999; each incorporated herein by reference.

In some embodiments, a polynucleotide molecule described herein may be a mixmer or comprise a mixmer sequence pattern. In some embodiments, mixmers can be polynucleotides that comprise both naturally and non-naturally occurring nucleosides or comprise two different types of non-naturally occurring nucleosides commonly in an alternating pattern. Mixmers may have higher binding affinity than unmodified polynucleotides and may be used, in particular, to specifically bind a target molecule, e.g., to block a binding site on the target molecule. In some embodiments, mixmers may not recruit an RNase to a target molecule and hence do not promote cleavage of the target molecule. Such polynucleotides that may be incapable of recruiting, e.g., RNase H have been described, e.g., see WO2007/112753 or WO2007/112754.

In some embodiments, a mixmer disclosed herein may comprise a repeating pattern of naturally occurring nucleosides and nucleoside analogues, or, e.g., one type of nucleoside analogue and a second type of nucleoside analogue. Yet, a mixmer need not comprise a repeating pattern and may instead comprise any arrangement of modified naturally occurring nucleosides and nucleosides or any arrangement of one type of modified nucleoside and a second type of modified nucleoside. Such repeating pattern, may, for example comprise every second or every third nucleoside as a modified nucleoside, e.g., LNA. In certain embodiments, the remaining nucleosides may be naturally occurring nucleosides, e.g., DNA, or may be a 2′ substituted nucleoside analogue, e.g., 2′ fluoro analogues or 2′-MOE, or any other some modified nucleoside(s) disclosed herein. It is understood that the repeating pattern of modified nucleoside, such as LNA units, may be combined with modified nucleoside at fixed positions (e.g., at the 5′ and/or 3′ termini).

In some embodiments, a mixmer may not comprise a region of more than 6. more than 5, more than 4, more than 3, or more than 2 consecutive naturally occurring nucleosides (e.g., DNA nucleosides). In some embodiments, the mixmer may comprise at least a region comprising at least two consecutive modified nucleosides, for example, at least two consecutive LNAs. In some embodiments, the mixmer may comprise at least a region consisting of at least three consecutive modified nucleoside units, e.g., at least three consecutive LNAs.

In some embodiments, the mixmer may not comprise a region of more than 8, more than 7, more than 6, more than 5, more than 4, more than 3, or more than 2 consecutive nucleoside analogues, e.g., LNAs. In some embodiments, LNA units may be replaced with other nucleoside analogues including, but not limited to, those referred to herein.

In some embodiments, mixmers may be designed to comprise a mixture of affinity enhancing modified nucleosides, such as, without limitation, in LNA nucleosides and 2′-O-Me nucleosides. In some embodiments, a mixmer may comprise modified internucleoside linkages (e.g., phosphorothioate internucleoside linkages or other linkages) between at least two, at least three, at least four, at least five, at least six or more nucleosides.

In some embodiments, a mixmer may comprise one or more morpholino nucleosides. In some embodiments, a mixmer may comprise morpholino nucleosides mixed (e.g., in an alternating manner) with one or more other nucleosides (e.g., DNA, RNA nucleosides) or modified nucleosides (e.g., 2′-O-Me nucleosides, LNA).

In some embodiments, mixmers may be useful for splice correcting or exon skipping, for example, as described in Chen S. et al., Molecules 2016, 21, 1582, Touznik A., et al., Scientific Reports, volume 7, Article number: 3672 (2017), the contents of each which are incorporated herein by reference.

A mixmer may be produced using any suitable method. Preparation of mixmers is described in, for example, U.S. Pat. No. 7,687,617, and U.S. Patent Application Publication Nos. US2012/0322851, US2009/0209748, US2009/0298916, US2006/0128646, and US2011/0077288. Additional examples of multimers are described, for example, in U.S. Pat. No. 5,693,773, US Patent Application Publication Nos. 2015/0247141; 2015/0315588; US 2011/0158937; the contents of each of which are incorporated herein by reference in their entireties.

In some embodiments, polynucleotide molecules comprising molecular cargos disclosed herein may comprise multimers (e.g., concatemers) of two or more polynucleotide molecules connected, e.g., by a linker. Polynucleotides in a multimer may be the same or different (e.g., targeting different sites on the same gene different genes or products thereof).

In some embodiments, multimers may comprise two or more polynucleotide molecules linked together by a cleavable linker. In some embodiments, multimers may comprise two or more polynucleotide molecules linked together, e.g., by a non-cleavable linker. In some embodiments, a multimer may comprise 2, 3, 4, 5, 6, 7, 8, 9, 10, 11, 12, 13, 14, 15, 16, 17, 18, 19, 20 or more polynucleotide molecules linked together. In some embodiments, a multimer may comprises 2 to 5, 2 to 10, 4 to 20 or 5 to 30 polynucleotide molecules linked together.

In some embodiments, a multimer may comprises two or more polynucleotide molecules linked in a linear arrangement, e.g., end-to-end. In some embodiments, a multimer may comprises two or more polynucleotide molecules linked end-to-end via a polynucleotide-based linker (e.g., an abasic linker, a poly-dT linker). In some embodiments, a multimer comprises a 3′ end of one polynucleotide linked to a 3′ end of another polynucleotide. In some embodiments, a multimer may comprise a 5′ end of one polynucleotide linked to a 3′ end of another polynucleotide. In some embodiments, a multimer comprises a 5′ end of one polynucleotide linked to a 5′ end of another polynucleotide. In some embodiments, multimers may comprise a branched structure comprising multiple polynucleotides linked together by a branching linker.

In some embodiments, a polynucleotide molecule of the present disclosure can target splicing. In some embodiments, the polynucleotide can targets splicing by inducing exon skipping and restoring the reading frame within a gene. For example, without limitation, the oligonucleotide may induce skipping of an exon encoding a frameshift mutation and/or an exon that encodes a premature stop codon. In some embodiments, a polynucleotide may induce exon skipping by, e.g., blocking spliceosome recognition of a splice site. In some embodiments, a polynucleotide molecule disclosed herein may induce inclusion of an exon by targeting a splice site inhibitory sequence. In some embodiments, the oligonucleotide promotes inclusion of a particular exon. In some embodiments, exon skipping results in a truncated but functional protein compared to the reference protein.

In some embodiments, the polynucleotide molecule described herein may be a messenger RNA (mRNA). mRNAs comprise an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. Bases of an mRNA can be modified bases such as pseudouridine, N-1-methyl-pseudouridine, or other naturally occurring or non-naturally occurring bases.

### Polypeptide Molecules

In some embodiments, the molecular cargo described herein comprises a polypeptide molecule. When a CACNG1-binding protein (e.g., antibody or antigen-binding fragment) described herein is covalently conjugated to a polypeptide molecule, such conjugates may also be referred to as “fusion proteins”. The term “fused” or “tethered” with regard to fused polypeptides refers to polypeptides joined directly or indirectly (e.g., via a linker or other polypeptide). In one embodiment, the fusion protein is encoded by a single nucleic acid that encodes the CACNG1-binding protein with the polypeptide molecule.

The anti-CACNG1 fusion proteins may be useful, for example, for delivery of the fused polypeptide molecule to various tissues (e.g., skeletal muscle tissue) and/or cells, including myofibers.

Non-limiting examples of polypeptide molecules that can be fused with a CACNG1-binding protein described herein can include, e.g., enzymes, molecules, or proteins, including other antigen-binding proteins (e.g., antibodies and antigen-binding fragments thereof).

In some embodiments, the present disclosure includes anti-CACNG1 fusion proteins, e.g., wherein the antigen-binding protein of the fusion is an antibody or antigen-binding fragment thereof set forth herein, and wherein the molecular cargo is a therapeutic agent useful for treating, preventing, or reducing the likelihood of a skeletal muscle disease or disorder disclosed herein.

In some embodiments, a muscle disease or disorder (e.g., a skeletal muscle disease or disorder) disclosed herein may include, muscular dystrophies (e.g., Duchenne muscular dystrophy (DMD), Becker muscular dystrophy (BMD), congenital muscular dystrophy, distal muscular dystrophy, Emery-Dreifuss muscular dystrophy, facioscapulohumeral muscular dystrophy, Limb-Girdle muscular dystrophy, myotonic muscular dystrophy, and oculopharyngeal muscular dystrophy), muscle atrophies (e.g., spinal muscular atrophies [e.g., Amyotrophic Lateral Sclerosis (ALS), infantile progressive spinal muscular atrophy, intermediate spinal muscular atrophy, juvenile spinal muscular atrophy, adult spinal muscular atrophy] as well as muscle atrophies induced by cancer cachexia, disuse, heart failure, chronic obstructive pulmonary disease, chronic infection, and the like), inflammatory myopathies (e.g., dermatomyositis, polymyositis, inclusion body myositis), diseases of peripheral nerve (e.g., Charcot-Marie tooth disease, Dejerine-Sottas disease, Friedreich's ataxia), diseases of the neuromuscular junction (e.g., Myasthenia gravis, Lambert-Eaton syndrome, botulism), metabolic diseases of the muscle (e.g., acid maltase deficiency, carnitine deficiency, carnitine palmityl transferase deficiency, debrancher enzyme deficiency, lactate dehydrogenase deficiency, mitochondrial myopathy, myoadenylate deaminase deficiency, phosphorylase deficiency, phosphofructokinase deficiency, phosphoglycerate kinase deficiency), central core disease, hyperthyroid myopathy, myotonia congenita, myotubular myopathy, Nemaline myopathy, paramyotonia congenita, periodic paralysis-hypokalemic-hyperkalemic, centronuclear myopathy, Laing distal myopathy, and myofibrillar myopathy.

Example methods for preparing a fusion protein comprising an antigen-binding protein are described in, e.g., U.S. Pat. No. 11,208,458, US Patent Publication No. US 2019/0112588, and Baik et al., Mol Ther. 2021 Dec. 1; 29(12):3512-3524; the contents of all of which are incorporated herein by reference in their entireties.

The CACNG1-binding proteins may also be fused to other polypeptide molecules such as, but are not limited to, an epitope (e.g., FLAG) or a tag sequence (e.g., His₆ (SEQ ID NO: 365, and the like) to allow for the detection and/or isolation of the anti-CACNG1 antigen-binding protein; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region (e.g., an Fc domain); a half-life extending polypeptide (e.g., albumin or albumin-binding peptides/proteins); a functional or non-functional antibody, or a heavy or light chain thereof; and a polypeptide which has an activity, such as a therapeutic activity, different from the anti-CACNG1 antigen-binding protein of the present disclosure.

In some embodiments, the polypeptide molecule can be a gene editing nuclease, such as Cas protein, ZFN, TALEN. Gene editing nucleases are described in further details below.

In some embodiments, anti-CACNG1 fusion proteins can be made by fusing the heterologous polypeptide molecule at either the N-terminus or at the C-terminus of the anti-CACNG1 antigen-binding protein (e.g., the heavy chain and/or light chain). Heterologous sequences can be fused either directly to the anti-CACNG1 antigen-binding protein, either chemically or by recombinant expression from a single polynucleotide or they may be joined via a linker or adapter molecule. A peptidyl linker or adapter molecule can be one or more amino acid residues (or -mers), e.g., 1, 2, 3, 4, 5, 6, 7, 8, or 9 residues (or -mers), preferably from 10 to 50 amino acid residues (or -mers), e.g., 10, 11, 12, 13, 14, 15, 16, 17, 18, 19, 20, 25, 30, 35, 40, 45, or 50 residues (or -mers), and more preferably from 15 to 35 amino acid residues (or -mers). A linker or adapter molecule can also be designed with a cleavage site for a protease to allow for the separation of the fused moieties.

When forming the fusion proteins of the present disclosure, a linker can be employed. The linker can be made up of amino acids linked together by peptide bonds, i.e., a peptidyl linker. In some embodiments, the linker is made up of from 1 to 20 or more amino acids linked by peptide bonds, wherein the amino acids are selected from the 20 naturally occurring amino acids. In some embodiments, the amino acids are selected from the amino acids glycine, serine, and glutamate. In some embodiments, suitable linkers include, for example, GSGEGEGSEGSG (SEQ ID NO: 381); GGSEGEGSEGGS (SEQ ID NO: 382); GGGGS (SEQ ID NO: 383); and GGGS (SEQ ID NO: 384). The present disclosure contemplates linkers of any length or composition.

### Liposomes, Lipid Nanoparticles and Other Carriers

In some embodiments, a conjugated molecular cargo described herein comprises a carrier, for example, a lipid-based carrier, such as a lipid nanoparticle (LNP), a liposome, a lipidoid, or a lipoplex, a polymeric nanoparticle, an inorganic nanoparticle, a peptide carrier, a nanoparticle mimic, or a nanotube.

In some embodiments, a conjugated molecular cargo described herein comprises a liposome or LNP. Liposomes and LNPs are vesicles including one or more lipid bilayers. In some embodiments, a liposome or LNP includes two or more concentric bilayers separated by aqueous compartments. Lipid bilayers can be functionalized and/or crosslinked to one another. Lipid bilayers can include one or more proteins, polysaccharides or other molecules.

Lipid formulations can protect biological molecules from degradation while improving their cellular uptake. Liposomes or LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such liposomes or LNPs can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine (DSPC). In another example, the other components can comprise a helper lipid such as cholesterol, an optional neutral lipid such as DSPC, and a stealth lipid such as S010, S024, S027, S031, or S033.

Liposomes are amphiphilic lipids which can form bilayers in an aqueous environment to encapsulate an aqueous core. The polypeptide (e.g., Cas protein) or polynucleotide (e.g., guide RNA) may be incorporated into the aqueous core. These lipids can have an anionic, cationic or zwitterionic hydrophilic head group. Liposomes can be formed from a single lipid or from a mixture of lipids. A mixture may comprise (1) a mixture of anionic lipids; (2) a mixture of cationic lipids; (3) a mixture of zwitterionic lipids; (4) a mixture of anionic lipids and cationic lipids; (5) a mixture of anionic lipids and zwitterionic lipids; (6) a mixture of zwitterionic lipids and cationic lipids; or (7) a mixture of anionic lipids, cationic lipids and zwitterionic lipids. Similarly, a mixture may comprise both saturated and unsaturated lipids. Exemplary phospholipids include, but are not limited to, phosphatidylethanolamines, phosphatidylcholines, phosphatidylserines, and phosphatidylglycerols. Cationic lipids include, but are not limited to, 1,2-distearyloxy-N,N-dimethyl-3-aminopropane (DSDMA), dioleoyl trimethylammonium propane (DOTAP), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids include dodecylphosphocholine, DPPC, and DOPC.

The liposomes or LNPs may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes.

In some examples, the liposomes or LNPs comprise cationic lipids. In some examples, the liposomes or LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Additional suitable cationic lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C18 alkyl chains, ether linkages between the head group and alkyl chains, and 0 to 3 double bonds. Such lipids include, e.g., DSDMA, DLinDMA, DLenDMA, and DODMA. The cationic lipids may comprise ether linkages and pH titratable head groups. Such lipids include, e.g., DODMA. Additional cationic lipids are described in U.S. Pat. Nos. 7,745,651; 5,208,036; 5,264,618; 5,279,833; 5,283,185; 5,753,613; and 5,785,992, incorporated herein by reference.

In some embodiments, the cationic lipids may comprise a protonatable tertiary amine head group. Such lipids are referred to herein as ionizable lipids. Ionizable lipids refer to lipid species comprising an ionizable amine head group and typically comprising a pKa of less than about 7. In environments with an acidic pH, the ionizable amine head group is protonated such that the ionizable lipid preferentially interacts with negatively charged molecules (e.g., nucleic acids such as the recombinant polynucleotides described herein) thus facilitating liposome or LNP assembly and encapsulation. Therefore, in some embodiments, ionizable lipids can increase the loading of nucleic acids into liposomes or LNPs. In environments where the pH is greater than about 7 (e.g., physiologic pH of 7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle.

In some embodiments, the liposomes or LNPs may comprise one or more non-cationic helper lipids. Exemplary helper lipids include (1,2-dilauroyl-sn-glycero-3-phosphoethanolamine) (DLPE), 1,2-diphytanoyl-sn-glycero-3-phosphoethanolamine (D iPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-distearoyl-sn-glycero-3-phosphoethanolamine (DSPE), ceramides, sphingomyelins, and cholesterol.

Some such lipids suitable for use in the liposomes or LNPs described herein are biodegradable in vivo. Examples of biodegradable lipids include, but are not limited to, (9Z,12Z)-3-((4,4-bis(octyloxy)butanoyl)oxy)-2-((((3-20 (diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate) or another ionizable lipid. See, e.g., PCT Publication Nos. WO2017/173054, WO2015/095340, and WO2014/136086. In some embodiments, the term cationic and ionizable in the context of liposome or LNP lipids is interchangeable, e.g., wherein ionizable lipids are cationic depending on the pH.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the liposomes or LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the liposomes or LNPs. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain liposome or LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (I-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000](PEG2k-DSA). In one particular example, the stealth lipid may be PEG2k-DMG.

In some embodiments, the liposomes or LNPs may further comprise one or more of PEG-modified lipids that comprise a poly(ethylene)glycol chain of up to 5 kDa in length covalently attached to a lipid comprising one or more C6-C20 alkyls. In some embodiments, the liposomes or LNPs further comprise 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)] (DSPE-PEG-amine). In some embodiments, the PEG-modified lipid comprises about 0.1% to about 1% of the total lipid content in a lipid nanoparticle. In some embodiments, the PEG-modified lipid comprises about 0.1%, about 0.2% about 0.3%, about 0.4%, about 0.5%, about 0.6%, about 0.7%, about 0.8%, about 0.9%, or about 1.0%, of the total lipid content in the liposome or lipid nanoparticle.

In some embodiments, a liposome or LNP described herein may comprise a conjugated lipid that inhibits aggregation of lipid particles. Such lipid conjugates include, but are not limited to, PEG-lipid conjugates such as, e.g, PEG coupled to dialkyloxypropyls (e.g, PEG-DAA conjugates), PEG coupled to diacylglycerols (e.g, PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates), polyamide oligomers (e.g, ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain embodiments, non-ester containing linker moieties, such as amides or carbamates, are used.

The liposomes or LNPs can comprise different respective molar ratios of the component lipids in the formulation. The mol-% of the CCD lipid may be, for example, from about 30 mol-% to about 60 mol-%. The mol-% of the helper lipid may be, for example, from about 30 mol-% to about 60 mol-%. The mol-% of the neutral lipid may be, for example, from about 1 mol-% to about 20 mol-%. The mol-% of the stealth lipid may be, for example, from about 1 mol-% to about 10 mol-%

The liposomes or LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100. The N/P ratio can also be from about 4 to about 6.

In some embodiments, the liposome or LNP can comprise a nuclease agent (e.g., CRISPR/Cas system, ZFN, or TALEN), can comprise a polynucleotide molecule (e.g., guide RNA), can comprise a nucleic acid construct encoding a polypeptide of interest (e.g., multidomain therapeutic protein), or can comprise both a nuclease agent (e.g., a CRISPR/Cas system) and a nucleic acid construct encoding a polypeptide of interest (e.g., a donor template for use in gene editing). Regarding CRISPR/Cas systems, the liposomes or LNPs can comprise the Cas protein in any form (e.g., protein, DNA, or mRNA) and/or can comprise the guide RNA(s) in any form (e.g., DNA or RNA). In one example, the liposomes or LNPs comprise the Cas protein in the form of mRNA (e.g., a modified RNA as described herein) and the guide RNA(s) in the form of RNA (e.g., a modified guide RNA as disclosed herein). As another example, the liposomes or LNPs can comprise the Cas protein in the form of protein and the guide RNA(s) in the form of RNA). In one example, the guide RNA and the Cas protein are each introduced in the form of RNA via LNP-mediated delivery in the same LNP. As discussed in more detail elsewhere herein, one or more of the RNAs can be modified.

For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity.

In certain liposomes or LNPs, the cargo can include a guide RNA or a nucleic acid encoding a guide RNA. In certain liposomes or LNPs, the cargo can include an mRNA encoding a Cas nuclease, such as Cas9, and a guide RNA or a nucleic acid encoding a guide RNA. In certain liposomes or LNPs, the cargo can include a nucleic acid construct encoding a polypeptide of interest (e.g., multidomain therapeutic protein) as described elsewhere herein. In certain liposomes or LNPs, the cargo can include an mRNA encoding a Cas nuclease, such as Cas9, a guide RNA or a nucleic acid encoding a guide RNA, and a nucleic acid construct encoding a polypeptide of interest (e.g., multidomain therapeutic protein). In some liposomes or LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG.

In some liposomes or LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25. Alternatively, the liposome or LNP formulation can include a ratio of Cas mRNA to gRNA nucleic acid of from about 2:1 to about 1:2. In specific examples, the ratio of Cas mRNA to gRNA can be about 2:1.

In some liposomes or LNPs, the cargo can comprise a nucleic acid construct encoding a polypeptide of interest (e.g., multidomain therapeutic protein) and gRNA. The nucleic acid construct encoding a polypeptide of interest (e.g., multidomain therapeutic protein) and gRNAs can be in different ratios. For example, the liposome or LNP formulation can include a ratio of nucleic acid construct to gRNA nucleic acid ranging from about 25:1 to about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

Dynamic Light Scattering (“DLS”) can be used to characterize the polydispersity index (“PDI”) and size of the liposomes and LNPs. In some embodiments, the PDI may range from about 0.005 to about 0.75. In some embodiments, the PDI may range from about 0.01 to about 0.5. In some embodiments, the PDI may range from about 0.02 to about 0.4. In some embodiments, the PDI may range from about 0.03 to about 0.35. In some embodiments, the PDI may range from about 0.1 to about 0.35.

The LNPs disclosed herein may have a size of about 1 to about 250 nm. In some embodiments, the LNPs may have a size of about 10 to about 200 nm. In some embodiments, the LNPs may have a size of about 20 to about 150 nm. In some embodiments, the LNPs may have a size of about 50 to about 150 nm. In some embodiments, the LNPs may have a size of about 50 to about 100 nm. In some embodiments, the LNPs may have a size of about 50 to about 120 nm. In some embodiments, the LNPs may have a size of about 75 to about 150 nm. In some embodiments, the LNPs may have a size of about 30 to about 200 nm. In some embodiments, the average sizes (diameters) of the fully formed nanoparticles are measured by dynamic light scattering on a Malvern Zetasizer (e.g., the nanoparticle sample may be diluted in phosphate buffered saline (PBS) so that the count rate is approximately 200-400 kcts, and the data may be presented as a weighted-average of the intensity measure).

In some embodiments, the liposomes or LNPs may be formed with an average encapsulation efficiency ranging from about 50% to about 100%. In some embodiments, the liposomes or LNPs may be formed with an average encapsulation efficiency ranging from about 50% to about 70%. In some embodiments, the liposomes or LNPs may be formed with an average encapsulation efficiency ranging from about 70% to about 90%. In some embodiments, the liposomes or LNPs may be formed with an average encapsulation efficiency ranging from about 90% to about 100%. In some embodiments, the liposomes or LNPs may be formed with an average encapsulation efficiency ranging from about 75% to about 95%.

In addition to liposomes and LNPs, a CACNG1-binding protein disclosed herein, such as an scFv or an antibody or an antigen-binding fragment thereof, may be conjugated to other carriers for delivery of nucleic acid and/protein molecules. Examples of other suitable carriers include, but are not limited to, lipoids and lipoplexes, particulate or polymeric nanoparticles, inorganic nanoparticles, peptide carriers, nanoparticle mimics, nanotubes, conjugates, immune stimulating complexes (ISCOM), virus-like particles (VLPs), self-assembling proteins, or emulsion delivery systems such as cationic submicron oil-in-water emulsions.

Polymeric microparticles or nanoparticles can also be used to encapsulate or adsorb a polypeptide (e.g., Cas protein) or polynucleotide (e.g., guide RNA). The particles may be substantially non-toxic and biodegradable. The particles useful for delivering a polynucleotide (e.g., guide RNA) may have an optimal size and zeta potential. For example, the microparticles may have a diameter in the range of 0.02 μm to 8 μm. In the instances when the composition has a population of micro- or nanoparticles with different diameters, at least 80%, 85%, 90%, or 95% of those particles ideally have diameters in the range of 0.03-7 μm. The particles may also have a zeta potential of between 40-100 mV, in order to provide maximal adsorption of the polynucleotide (e.g., guide RNA) to the particles.

Non-toxic and biodegradable polymers include, but are not limited to, poly(ahydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, one or more natural polymers such as a polysaccharide, for example pullulan, alginate, inulin, and chitosan, and combinations thereof. In some embodiments, the particles are formed from poly(ahydroxy acids), such as a poly(lactides) (PLA), poly(g-glutamic acid) (g-PGA), poly(ethylene glycol) (PEG), polystyrene, copolymers of lactide and glycolide such as a poly(D,L-lactide-co-glycolide) (PLG), and copolymers of D,L-lactide and caprolactone. Useful PLG polymers can include those having a lactide/glycolide molar ratio ranging, for example, from 20:80 to 80:20 e.g., 25:75, 40:60, 45:55, 55:45, 60:40, 75:25. Useful PLG polymers include those having a molecular weight between, for example, 5,000-200,000 Da e.g., between 10,000-100,000, 20,000-70,000, 40,000-50,000 Da.

The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles.

For example, the inorganic nanoparticles may be calcium phosphate nanoparticles, silicon nanoparticles or gold nanoparticles. Inorganic nanoparticles typically have a rigid structure and comprise a shell in which a polypeptide or polynucleotide is encapsulated or a core to which the polypeptide or polynucleotide may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

Other molecules suitable for complexing with the polypeptides or polynucleotides of the disclosure include cationic molecules, such as, polyamidoamine, dendritic polylysine, polyethylene irinine or polypropylene imine, polylysine, chitosan, DNA-gelatin coarcervates, DEAE dextran, dendrimers, or polyethylenimine (PEI).

In some embodiments, polypeptides or polynucleotides of the present disclosure can be conjugated to nanoparticles. Nanoparticles that may be used for conjugation with antigens and/or antibodies of the present disclosure include but not are limited to chitosan-shelled nanoparticles, carbon nanotubes, PEGylated liposomes, poly(d,1-lactide-co-glycolide)/montmorillonite (PLGA/MMT) nanoparticles, poly(lactide-co-glycolide) (PLGA) nanoparticles, poly-(malic acid)-based nanoparticles, and other inorganic nanoparticles (e.g., nanoparticles made of magnesium-aluminium layered double hydroxides with disuccinimidyl carbonate (DSC), and TiO₂ nanoparticles). Nanoparticles can be developed and conjugated to an antigens and/or antibodies contained in a composition for targeting virus-infected cells.

Oil-in-water emulsions may also be used for delivering a polypeptide or polynucleotide (e.g., mRNA) to a subject. Examples of oils useful for making the emulsions include animal (e.g., fish) oil or vegetable oil (e.g., nuts, grains and seeds). The oil may be biodegradable and biocompatible. Exemplary oils include, but are not limited to, tocopherols and squalene, a shark liver oil which is a branched, unsaturated terpenoid and combinations thereof. Terpenoids are branched chain oils that are synthesized biochemically in 5-carbon isoprene units.

The aqueous component of the emulsion can be water or can be water in which additional components have been added. For example, it may include salts to form a buffer e.g., citrate or phosphate salts, such as sodium salts. Exemplary buffers include a borate buffer, a citrate buffer, a histidine buffer a phosphate buffer, a Tris buffer, or a succinate buffer.

In some embodiments, the oil-in water emulsions include one or more cationic molecules. For example, a cationic lipid can be included in the emulsion to provide a positively charged droplet surface to which negatively-charged polynucleotide (e.g., mRNA) can attach. Exemplary cationic lipids include, but are not limited to: 1,2-dioleoyloxy-3-(trimethylammonio)propane (DOTAP), 1,2-Dimyristoyl-3-Trimethyl-AmmoniumPropane (DMTAP), 3′-[N—(N′,N′-Dimethylaminoethane)-carbamoyl]Cholesterol (DC Cholesterol), dimethyldioctadecyl-ammonium (DDA e.g., the bromide), dipalmitoyl(C16:0)trimethyl ammonium propane (DPTAP), distearoyltrimethylammonium propane (DSTAP). Other useful cationic lipids include benzalkonium chloride (BAK), benzethonium chloride, cholesterol hemisuccinate choline ester, lipopolyamines (e.g., dioctadecylamidoglycylspermine (DOGS), dipalmitoyl phosphatidylethanol-amidospermine (DPPES)), cetramide, cetylpyridinium chloride (CPC), cetyl trimethylammonium chloride (CTAC), cationic derivatives of cholesterol (e.g., cholesteryl-3.beta.-oxysuccinamidoethylenetrimethylammonium salt, cholesteryl-3.beta.-oxysuccinamidoethylene-dimethylamine, cholesteryl-3.beta.-carboxyamidoethylenetrimethylammonium salt, and cholesteryl-3.beta.-carboxyamidoethylenedimethylamine), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), cholesteryl (4′-trimethylammonio) butanoate), N-alkyl pyridinium salts (e.g., cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-alpha.dioleoylphosphatidylethanolamine, lipopoly-L (or D)-lysine (LPLL, LPDL), poly(L (or D)-lysine conjugated to N-glutarylphosphatidylethanolamine, dialkyldimethylammonium salts, [1-(2,3-dioleyloxy)-propyl]-N,N,N,trimethylammonium chloride, 1,2-diacyl-3-(trimethylammonio) propane (acyl group can be dimyristoyl, dipalmitoyl, distearoyl, or dioleoyl), 1,2-diacyl-3 (dimethylammonio)propane (acyl group can be dimyristoyl, dipalmitoyl, distearoyl, or dioleoyl), 1,2-dioleoyl-3-(4′-trimethyl-ammonio)butanoyl-sn-glycerol, 1,2-dioleoyl 3-succinyl-sn-glycerol choline ester, didodecyl glutamate ester with pendant amino group (C GluPhCnN), and ditetradecyl glutamate ester with pendant amino group (C14GluCnN+).

In some embodiments, in addition to the oil and cationic lipid, an emulsion can also include a non-ionic surfactant and/or a zwitterionic surfactant. Examples of useful surfactants include, but are not limited to: the polyoxyethylene sorbitan esters surfactants, e.g., polysorbate 20 and polysorbate 80; copolymers of ethylene oxide, propylene oxide, and/or butylene oxide, linear block copolymers; phospholipids, e.g., phosphatidylcholine; polyoxyethylene fatty ethers derived from lauryl, cetyl, stearyl and oleyl alcohols; polyoxyethylene-9-lauryl ether; octoxynols; (octylphenoxy)polyethoxyethanol; and sorbitan esters.

In some embodiments, a polynucleotide described herein may be incorporated into polynucleotide complexes, such as, but not limited to, nanoparticles (e.g., polynucleotide self-assembled nanoparticles, polymer-based self-assembled nanoparticles, inorganic nanoparticles, lipid nanoparticles, semiconductive/metallic nanoparticles), gels and hydrogels, polynucleotide complexes with cations and anions, microparticles, and any combination thereof. The polynucleotide complexes may be conjugated to a CACNG1-binding protein described herein, e.g., via linkage to the polynucleotide or nanoparticle/hydrogel/microparticle.

In some embodiments, the polynucleotides disclosed herein may be formulated as self-assembled nanoparticles. As a non-limiting example, polynucleotides may be used to make nanoparticles which may be used in a delivery system for the polynucleotides (See e.g., PCT Publication No. WO2012/125987). In some embodiments, the polynucleotide self-assembled nanoparticles may comprise a core of the polynucleotides disclosed herein and a polymer shell. The polymer shell may be any of the polymers described herein and are known in the art. In an additional embodiment, the polymer shell may be used to protect the polynucleotides in the core.

In some embodiments, self-assembled nanoparticles may be microsponges formed of long polymers of polynucleotide hairpins which form into crystalline “pleated” sheets before self-assembling into microsponges. These microsponges are densely-packed sponge like microparticles which may function as an efficient carrier and may be able to deliver cargo to a cell. The microsponges may be from 1 μm to 300 nm in diameter. The microsponges may be complexed with other agents known in the art to form larger microsponges. As a non-limiting example, the microsponge may be complexed with an agent to form an outer layer to promote cellular uptake such as polycation polyethyleneime (PEI). This complex can form a 250-nm diameter particle that can remain stable at high temperatures (150° C.) (Grabow and Jaegar, Nature Materials 2012, 11:269-269). Additionally, these microsponges may be able to exhibit an extraordinary degree of protection from degradation by ribonucleases. In an embodiment, the polymer-based self-assembled nanoparticles such as, but not limited to, microsponges, may be fully programmable nanoparticles. The geometry, size and stoichiometry of the nanoparticle may be precisely controlled to create the optimal nanoparticle for delivery of cargo such as, but not limited to, polynucleotides.

In some embodiments, a polynucleotide disclosed herein may be formulated in inorganic nanoparticles (see U.S. Pat. No. 8,257,745). The inorganic nanoparticles may include, but are not limited to, clay substances that are water swellable. As a non-limiting example, the inorganic nanoparticle may include synthetic smectite clays which are made from simple silicates (See U.S. Pat. Nos. 5,585,108 and 8,257,745).

In some embodiments, a polynucleotide disclosed herein may be formulated in water-dispersible nanoparticle comprising a semiconductive or metallic material (U.S. Patent Application Publication No. 2012/0228565; herein incorporated by reference in its entirety) or formed in a magnetic nanoparticle (U.S. Patent Application Publication No. 2012/0265001 and 2012/0283503). The water-dispersible nanoparticles may be hydrophobic nanoparticles or hydrophilic nanoparticles.

In some embodiments, the polynucleotides disclosed herein may be encapsulated into any hydrogel known in the art which may form a gel when injected into a subject. Hydrogels are a network of polymer chains that are hydrophilic, and are sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. The hydrogel described herein may be used to encapsulate lipid nanoparticles which are biocompatible, biodegradable and/or porous.

As a non-limiting example, the hydrogel may be an aptamer-functionalized hydrogel. The aptamer-functionalized hydrogel may be programmed to release one or more polynucleotides using polynucleotide hybridization. (Battig et al., J. Am. Chem. Society. 2012 134:12410⁻¹²⁴¹³). In some embodiments, the polynucleotide may be encapsulated in a lipid nanoparticle and then the lipid nanoparticle may be encapsulated into a hydrogel.

In some embodiments, the polynucleotides disclosed herein may be encapsulated into a fibrin gel, fibrin hydrogel or fibrin glue. In another embodiment, the polynucleotides may be formulated in a lipid nanoparticle or a rapidly eliminated lipid nanoparticle prior to being encapsulated into a fibrin gel, fibrin hydrogel or a fibrin glue. In yet another embodiment, the polynucleotides may be formulated as a lipoplex prior to being encapsulated into a fibrin gel, hydrogel or a fibrin glue. Fibrin gels, hydrogels and glues comprise two components, a fibrinogen solution and a thrombin solution which is rich in calcium (See e.g., Spicer and Mikos, Journal of Controlled Release 2010. 148: 49-55; Kidd et al. Journal of Controlled Release 2012. 157:80-85). The concentration of the components of the fibrin gel, hydrogel and/or glue can be altered to change the characteristics, the network mesh size, and/or the degradation characteristics of the gel, hydrogel and/or glue such as, but not limited to changing the release characteristics of the fibrin gel, hydrogel and/or glue. (See e.g., Spicer and Mikos, Journal of Controlled Release 2010. 148: 49-55; Kidd et al. Journal of Controlled Release 2012. 157:80-85; Catelas et al. Tissue Engineering 2008. 14:119-128). This feature may be advantageous when used to deliver the polynucleotide disclosed herein. (See e.g., Kidd et al. Journal of Controlled Release 2012. 157:80-85; Catelas et al. Tissue Engineering 2008. 14:119-128).

In some embodiments, a polynucleotide disclosed herein may include cations or anions. In one embodiment, the formulations include metal cations such as, but not limited to, Zn²⁺, Ca²⁺, Cu²⁺, Mg²⁺ and combinations thereof. As a non-limiting example, formulations may include polymers and a polynucleotide complexed with a metal cation (See U.S. Pat. Nos. 6,265,389 and 6,555,525).

In some embodiments, a polynucleotide may be formulated in nanoparticles and/or microparticles. These nanoparticles and/or microparticles may be molded into any size shape and chemistry. As an example, the nanoparticles and/or microparticles may be made using the PRINT® technology by LIQUIDA TECHNOLOGIES (Morrisville, N.C.) (See e.g., International Pub. Publication No. WO2007/024323).

In some embodiments, the polynucleotides disclosed herein may be formulated in NanoJackets and NanoLiposomes by Keystone Nano (State College, Pa.). NanoJackets are made of compounds that are naturally found in the body including calcium, phosphate and may also include a small amount of silicates. Nanojackets may range in size from 5 to 50 nm and may be used to deliver hydrophilic and hydrophobic compounds such as, but not limited to, polynucleotides, primary constructs and/or polynucleotide. NanoLiposomes are made of lipids such as, but not limited to, lipids which naturally occur in the body. NanoLiposomes may range in size from 60-80 nm and may be used to deliver hydrophilic and hydrophobic compounds such as, but not limited to, polynucleotides, primary constructs and/or polynucleotide. In one aspect, the polynucleotides disclosed herein are formulated in a NanoLiposome such as, but not limited to, Ceramide NanoLiposomes.

### Gene Editing System

In various embodiments, a molecular cargo described herein can include a gene editing system or components of such systems. Various known gene editing systems can be used in the practice of the present methods and compositions described herein, including, e.g., a Clustered Regularly Interspersed Short Palindromic Repeats (CRISPR)/Cas system; zinc finger nuclease (ZFN) system; transcription activator-like effector nuclease (TALEN) system, or systems using meganucleases, restriction endonucleases, or recombinases. Generally, these gene editing systems are used to modify a genome within a cell by inducing a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA (gRNA) to guide specific cleavage or nicking of a target DNA sequence. Further, targeted nucleases have been developed, and additional nucleases are being developed, for example based on the Argonaute system (e.g., from T. thermophilus, known as ‘TtAgo’, see Swarts et al (2014) Nature 507(7491): 258-261), which also may have the potential for uses in genome editing and gene therapy.

Deletion of DNA may be performed using a gene editing system to knock-out or disrupt a target gene. A knock-out can be a gene knock-down or the gene can be knocked out by a mutation such as, a point mutation, an insertion, a deletion, a frameshift, or a missense mutation by techniques known in the art. Alternatively, a knock-in of an exogenous gene or replacement of a defective gene with a corrective gene can also be achieved with a gene editing system. In such instances, a donor template carrying an heterologous gene to be inserted into a genomic locus is provided along with a gene editing system. The donor template would typically include homology arms corresponding to the genomic locus which is targeted by a gene editing system.

There are various ways to incorporate a gene editing system or component(s) thereof (e.g., Cas protein, guide RNA) to an anti-CACNG1 protein-drug conjugate described herein. In some embodiments, a gene editing system or component(s) thereof (e.g., Cas protein or nucleic acid (e.g., mRNA or DNA) encoding, guide RNA or DNA encoding) are loaded to a carrier described, such as a liposome or LNP, which is conjugated to a CACNG1-binding protein described herein. In some embodiments, a guide RNA or a DNA encoding the guide RNA is conjugated to a CACNG1-binding protein described herein. In some embodiments, a gene editing nuclease (e.g., Cas protein, ZFN, TALEN) or one or more nucleic acids (e.g., mRNA or DNA) encoding the gene editing nuclease is conjugated to CACNG1-binding protein described herein. In some embodiments, both a guide RNA (or DNA encoding) and a Cas protein (or nucleic acid (e.g., mRNA or DNA) encoding) may be conjugated to a CACNG1-binding protein described herein. In some embodiments, a guide RNA (or DNA encoding) is conjugated to a CACNG1-binding protein described herein, and a Cas protein (or nucleic acid (e.g., mRNA or DNA) encoding) is loaded to a carrier described, such as a liposome or LNP, which is conjugated to a CACNG1-binding protein described herein. In some embodiments, a Cas protein (or nucleic acid (e.g., mRNA or DNA) encoding) is conjugated to a CACNG1-binding protein described herein, and a guide RNA (or DNA encoding) is loaded to a carrier described, such as a liposome or LNP, which is conjugated to a CACNG1-binding protein described herein.

In some embodiments, the molecular cargo disclosed herein can comprise a CRISPR/Cas system or components of such systems. CRISPR/Cas systems include transcripts and other elements involved in the expression of, or directing the activity of, Cas genes. A CRISPR/Cas system can be, for example, a type I, a type II, or a type III system. Alternatively, a CRISPR/Cas system can be a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed cleavage of nucleic acids.

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18^{th} base pair from the PAM sequence on the non-targeted strand and after the 23^{rd} base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacterjejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCΔΔCt C, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number AOQ7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1 180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas nuclease can be fused to a cleavage domain, an epigenetic modification domain, a transcriptional activation domain, or a transcriptional repressor domain. See WO 2014/089290, herein incorporated by reference in its entirety for all purposes. Examples of transcriptional activation domains include a herpes simplex virus VP 16 activation domain, VP64 (which is a tetrameric derivative of VP 16), a NFKB p65 activation domain, p53 activation domains 1 and 2, a CREB (cAMP response element binding protein) activation domain, an E2A activation domain, and an NFAT (nuclear factor of activated T-cells) activation domain. Other examples include activation domains from Octl, Oct-2A, SP1, AP-2, CTF1, P300, CBP, PCAF, SRC1, PvALF, ERF-2, OsGAI, HALF-1, Cl, API, ARF-5, ARF-6, ARF-7, ARF-8, CPRF1, CPRF4, MYC-RP/GP, TRAB1PC4, and HSF1. See, e.g., US 2016/0237456, EP3045537, and WO 2011/146121, each of which is incorporated by reference in its entirety for all purposes.

In some cases, a transcriptional activation system can be used comprising a dCas9-VP64 fusion protein paired with MS2-p65-HSFI. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes.

Examples of transcriptional repressor domains include inducible cAMP early repressor (ICER) domains, Kruppel-associated box A (KRAB-A) repressor domains, YY 1 glycine rich repressor domains, Spl-like repressors, E(spl) repressors, IKB repressor, and MeCP2. Other examples include transcriptional repressor domains from A/B, KOX, TGF-beta-inducible early gene (TIEG), v-erbA, SID, SID4X, MBD2, MBD3, DNMT1, DNMG3A, DNMT3B, Rb, ROM2, See, e.g., EP3045537 and WO 2011/146121, each of which is incorporated by reference in its entirety for all purposes. Cas nucleases can also be fused to a heterologous polypeptide providing increased or decreased stability. The fused domain or heterologous polypeptide can be located at the N-terminus, the C-terminus, or internally within the Cas nuclease.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 385) or PKKKRRV (SEQ ID NO: 386). The NLS may be a bipartite sequence, such as the NLS of nucleoplasmin, KRPAATKKAGQAKKKK (SEQ ID NO: 387). In a specific example, a single PKKKRKV (SEQ ID NO: 385) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge). Examples of codon-optimized Cas9 coding sequences, Cas9 mRNAs, and Cas9 protein sequences include those described in WO2013/176772, WO2014/065596, WO2016/106121, and WO2019/067910 are hereby incorporated by reference. In particular, the Cas9 coding sequences and Cas9 amino acid sequences of the table at paragraph [0449] WO2019/067910, and the Cas9 mRNAs and coding sequences of paragraphs [0214]-[0234] of WO2019/067910 are hereby incorporated by reference. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA). Different promoters can be used to drive expression of the gRNA, such as a U6 promoter or the small tRNA Gln. Likewise, different promoters can be used to drive Cas9 expression.

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl pseudouridine can be used. As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). Likewise, Cas mRNAs can be modified by depletion of uridine using synonymous codons. For example, capped and polyadenylated Cas mRNA fully substituted with pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides (SEQ ID NO: 424).

In some embodiments, a CRISPR/Cas system can be used to create a site of insertion at a desired locus within a host genome, at which site a construct disclosed herein can be inserted to express one or more polypeptides of interest. Methods of designing suitable guide RNAs that target any desired locus of a host genome for insertion are well known in the art. A construct comprising a transgene may be heterologous with respect to its insertion site, for example, insertion of a heterologous transgene into a “safe harbor” locus. A construct comprising a transgene may be non-heterologous with respect to its insertion site, for example, insertion of a wild-type transgene into its endogenous locus.

Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1 (PPP1 R12C), Rosa (e.g., Rosa26), AngptiS, ApoC3, ASGR2, FIX (F9), G6PC, Gys2, HGD, Lp(a), Pcsk9, SERPINA1, TF, and TTR. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67, 328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In some embodiments, the heterologous gene may be inserted into a safe harbor locus and use the safe harbor locus's endogenous signal sequence. In some embodiments, the heterologous gene may comprise its own signal sequence, may be inserted into the safe harbor locus, and may further use the safe harbor locus's endogenous signal sequence. In some embodiments, the gene may comprise its own signal sequence and an internal ribosomal entry site (IRES), may be inserted into the safe harbor locus, and may further use the safe harbor locus's endogenous signal sequence. In some embodiments, the gene may comprise its own signal sequence and IRES, may be inserted into the safe harbor locus, and does not use the safe harbor locus's endogenous signal sequence. In some embodiments, the gene may be inserted into the safe harbor locus and may comprise an IRES and does not use any signal sequence.

In some methods, two or more nuclease agents can be used. For example, two or more nuclease agents can be used, each targeting a nuclease target sequence including or proximate to the start codon. As another example, two nuclease agents can be used, one targeting a nuclease target sequence including or proximate to the start codon, and one targeting a nuclease target sequence including or proximate to the stop codon, wherein cleavage by the nuclease agents can result in deletion of the coding region between the two nuclease target sequences. As yet another example, three or more nuclease agents can be used, with one or more (e.g., two) targeting nuclease target sequences including or proximate to the start codon, and one or more (e.g., two) targeting nuclease target sequences including or proximate to the stop codon, wherein cleavage by the nuclease agents can result in deletion of the coding region between the nuclease target sequences including or proximate to the start codon and the nuclease target sequence including or proximate to the stop codon.

In some embodiments, CRISPR/Cas systems used in the compositions and methods disclosed herein can be non-naturally occurring.

In some embodiments, the Cas protein (e.g., Cas9) may be complexed with a gRNA to form a ribonucleoprotein complex (RNP). In some embodiments, a molecular cargo (e.g., liposome or LNP) of the present disclosure comprises a ribonucleoprotein complex (RNP) comprising a Cas protein (e.g., Cas9) and a gRNA.

In some embodiments, a molecular cargo (e.g., liposomes and LNPs) described herein may comprise one or more components from gene editing systems other than a CRISPR/Cas system. In some embodiments, the molecular cargo is a nuclease, such as Zinc-finger nuclease (ZFN) or a TALEN, which is effective to bind and modify at a target gene.

Any nuclease molecular cargo that induces a nick or double-strand break into a desired target sequence or any DNA-binding protein that binds to a desired target sequence can be used in the methods and compositions disclosed herein. A naturally occurring or native nuclease molecular cargo can be employed so long as the nuclease molecular cargo induces a nick or double-strand break in a desired target sequence. Likewise, a naturally occurring or native DNA-binding protein can be employed so long as the DNA-binding protein binds to the desired target sequence. Alternatively, a modified or engineered nuclease molecular cargo or DNA-binding protein can be employed. An “engineered nuclease molecular cargo or DNA-binding protein” includes a nuclease molecular cargo or DNA-binding protein that is engineered (modified or derived) from its native form to specifically recognize a desired target sequence. Thus, an engineered nuclease molecular cargo or DNA-binding protein can be derived from a native, naturally occurring nuclease molecular cargo or DNA-binding protein or it can be artificially created or synthesized. The engineered nuclease molecular cargo or DNA-binding protein can recognize a target sequence, for example, wherein the target sequence is not a sequence that would have been recognized by a native (non-engineered or non-modified) nuclease molecular cargo or DNA-binding protein. The modification of the nuclease molecular cargo or DNA-binding protein can be as little as one amino acid in a protein cleavage molecular cargo or one nucleotide in a nucleic acid cleavage molecular cargo. Producing a nick or double-strand break in a target sequence or other DNA can be referred to herein as “cutting” or “cleaving” the target sequence or other DNA.

Active variants and fragments of nuclease molecular cargoes or DNA-binding proteins (i.e., an engineered nuclease molecular cargo or DNA-binding protein) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease molecular cargo or DNA-binding protein, wherein the active variants retain the ability to cut at a desired target sequence and hence retain nick or double-strand-break-inducing activity or retain the ability to bind a desired target sequence. For example, any of the nuclease molecular cargoes described herein can be modified from a native endonuclease sequence and designed to recognize and induce a nick or double-strand break at a target sequence that was not recognized by the native nuclease molecular cargo. Thus, some engineered nucleases have a specificity to induce a nick or double-strand break at a target sequence that is different from the corresponding native nuclease molecular cargo target sequence. Assays for nick or double-strand-break-inducing activity are known and generally measure the overall activity and specificity of the endonuclease on DNA substrates containing the target sequence. The target sequence can be endogenous (or native) to the cell or the target sequence can be exogenous to the cell. A target sequence that is exogenous to the cell is not naturally occurring in the genome of the cell. The target sequence can also exogenous to the polynucleotides of interest that one desires to be positioned at the target locus. In some cases, the target sequence is present only once in the genome of the host cell.

Active variants and fragments of the exemplified target sequences are also provided. Such active variants can comprise at least 65%, 70%, 75%, 80%, 85%, 90%, 91%, 92%, 93%, 94%, 95%, 96%, 97%, 98%, 99% or more sequence identity to the given target sequence, wherein the active variants retain biological activity and hence are capable of being recognized and cleaved by a nuclease molecular cargo in a sequence-specific manner. Assays to measure the double-strand break of a target sequence by a nuclease molecular cargo are known (e.g., TAQMAN® qPCR assay, Frendewey et al. (2010) Methods in Enzymology 476:295-307, herein incorporated by reference in its entirety for all purposes).

The length of the target sequence can vary, and includes, for example, target sequences that are about 30-36 bp for a zinc finger nuclease (ZFN) pair (about 15-18 bp for each ZFN), about 36 bp for a Transcription Activator-Like Effector (TALE) protein or Transcription Activator-Like Effector Nuclease (TALEN), or about 20 bp for a CRISPR/Cas9 guide RNA.

The target sequence of the DNA-binding protein or nuclease molecular cargo can be positioned anywhere in or near the target genomic locus. The target sequence can be located within a coding region of a gene, or within regulatory regions that influence the expression of the gene. A target sequence of the DNA-binding protein or nuclease molecular cargo can be located in an intron, an exon, a promoter, an enhancer, a regulatory region, or any non-protein coding region.

One type of DNA-binding protein that can be employed in the various methods and compositions disclosed herein is a Transcription Activator-Like Effector (TALE). A TALE can be fused or linked to, for example, an epigenetic modification domain, a transcriptional activation domain, or a transcriptional repressor domain. Examples of such domains are described with respect to Cas proteins, below, and can also be found, for example, in WO 2011/145121, herein incorporated by reference in its entirety for all purposes. Correspondingly, one type of nuclease molecular cargo that can be employed in the various methods and compositions disclosed herein is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease such as Fokl. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See WO 2010/079430; Morbitzer et al. (2010) Proc. Natl. Acad. Sci. U.S.A. 107(50:21617-21622; Scholze & Boch (2010) Virulence 1:428-432; Christian et al. (2010) Genetics 186:757-761; Li et al. (2011) Nucleic Acids Res. 39(l):359-372; and Miller et al. (2011) Nature Biotechnology 29: 143-148, each of which is herein incorporated by reference in its entirety for all purposes.

The non-specific DNA cleavage domain from the end of the Fokl endonuclease can be used to construct hybrid nucleases that are active in a yeast assay. These molecular cargoes are also active in plant cells and in animal cells. The Fokl domain functions as a dimer, requiring two constructs with unique DNA binding domains for sites in the target genome with proper orientation and spacing. Both the number of amino acid residues between the TALEN DNA binding domain and the Fokl cleavage domain and the number of bases between the two individual TALEN binding sites are parameters for achieving high levels of activity. The number of amino acid residues between the TALEN DNA binding domain and the Fokl cleavage domain may be modified by introduction of a spacer (distinct from the spacer sequence) between the plurality of TAL effector repeat sequences and the Fokl endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two-step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled, they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms.

Examples of suitable TAL nucleases, and methods for preparing suitable TAL nucleases, are disclosed, e.g., in US 2011/0239315 A1, US 2011/0269234 A1, US 2011/0145940 A1, US 2003/0232410 A1, US 2005/0208489 A1, US 2005/0026157 A1, US 2005/0064474 A1, US 2006/0188987 A1, and US 2006/0063231 A1, each of which is herein incorporated by reference in its entirety for all purposes. In some embodiments, TAL effector nucleases are engineered that cut in or near a target nucleic acid sequence in, for example, a genomic locus of interest, wherein the target nucleic acid sequence is at or near a sequence to be modified.

In some TALENs, each monomer of the TALEN comprises 33-35 TAL repeats that recognize a single base pair via two hypervariable residues. In some TALENs, the nuclease molecular cargo is a chimeric protein comprising a TAL-repeat-based DNA binding domain operably linked to an independent nuclease such as a Fokl endonuclease. For example, the nuclease molecular cargo can comprise a first TAL-repeat-based DNA binding domain and a second TAL-repeat-based DNA binding domain, wherein each of the first and the second TAL-repeat-based DNA binding domains is operably linked to a Fokl nuclease, wherein the first and the second TAL-repeat-based DNA binding domain recognize two contiguous target DNA sequences in each strand of the target DNA sequence separated by a spacer sequence of varying length (12-20 bp), and wherein the Fokl nuclease subunits dimerize to create an active nuclease that makes a double strand break at a target sequence.

Transcription Activator-Like Effector Nucleases (TALENs) are artificial restriction enzymes generated by fusing the TAL effector DNA binding domain to a DNA cleavage domain. These molecular cargoes enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Pat. Nos. 8,586,363; 8,450,471; 8,440,431; 8,440,432; and 8,697,853, all of which are incorporated by reference herein in their entirety.

Another example of a DNA-binding protein is a zinc finger protein. Such zinc finger proteins can be linked or fused to, for example, an epigenetic modification domain, a transcriptional activation domain, or a transcriptional repressor domain. Examples of such domains are described with respect to Cas proteins, below, and can also be found, for example, in WO 2011/145121, herein incorporated by reference in its entirety for all purposes. Correspondingly, another example of a nuclease molecular cargo that can be employed in the various methods and compositions disclosed herein is a zinc-finger nuclease (ZFN). In some ZFNs, each monomer of the ZFN comprises three or more zinc finger-based DNA binding domains, wherein each zinc finger-based DNA binding domain binds to a 3 bp subsite. In other ZFNs, the ZFN is a chimeric protein comprising a zinc finger-based DNA binding domain operably linked to an independent nuclease such as a Fokl endonuclease. For example, the nuclease molecular cargo can comprise a first ZFN and a second ZFN, wherein each of the first ZFN and the second ZFN is operably linked to a Fokl nuclease subunit, wherein the first and the second ZFN recognize two contiguous target DNA sequences in each strand of the target DNA sequence separated by about 5-7 bp spacer, and wherein the Fokl nuclease subunits dimerize to create an active nuclease that makes a double strand break. See, e.g., US 2006/0246567; US 2008/0182332; US 2002/0081614; US 2003/0021776; WO 2002/057308 A2; US 2013/0123484; US 2010/0291048; WO 2011/017293 A2; and Gaj et al. (2013) Trends in Biotechnology 31(7):397-405, each of which is herein incorporated by reference in its entirety for all purposes.

### Small Molecules or Detectable Moieties

In some embodiments, the molecular cargo comprises a small molecule as a therapeutic agent, e.g. a therapeutic agent that may be useful for treating muscle wasting or genetic muscle diseases. A small molecule (SM) can permeably enter or diffuse into cells. For example, without limitation, once a conjugate comprising a small molecule (e.g., a bioactive small molecule) described herein internalizes to inside a cell, the linker of the conjugate can be cleaved to release the bioactive small molecule which can then modulate intracellular bio-responses, such as, but not limited to, binding to a nuclear receptor (e.g., DHT binding to androgen receptor; budesonide binding to glucocorticoid receptor) or other proteins, and may, for example, cause cancer cells to die. This is different from many large molecular weight molecules such as antibodies. An example, of a small molecule may be conjugated to an anti-CACNG1 antigen-binding protein, to form an anti-CACNG1:SM conjugate.

Therapeutic agents that may be useful for treating muscle wasting or genetic muscle diseases include an androgen (e.g., testosterone and biologically active variants thereof or dihydrotestosterone (DHT)), a glucocorticoid and biologically active variants thereof (e.g., budesonide), β2-adrenergic receptor agonists (e.g., clenbuterol), rapamycin or its analogs, MAPK inhibitors, or histone deacetylase inhibitors, etc.

In some embodiments, the molecular cargo comprises a radioactive isotope, e.g., comprising a radionuclide. Accordingly, also provided herein are antibody-radionuclide conjugates (ARCs) comprising anti-hCACNG1 antibodies conjugated to one or more radionuclides. Exemplary radionuclides that can be used in the context of this aspect of the disclosure include, but are not limited to, e.g., ²²⁵Ac, ²¹²Bi, ²¹³Bi, ¹³¹I, ¹⁸⁶Re, ²²⁷Th, ²²²Rn, ²²³Ra, ²²⁴Ra, and ⁹⁰Y.

### Conjugation of Molecular Cargo to Antigen-binding Protein

In some embodiments, a molecular cargo described herein, e.g., a polynucleotide molecule described herein, or a liposome or LNP, is conjugated to a CACNG1-binding protein for delivery to a site of interest (e.g., skeletal muscle tissue). In some embodiments, the CACNG1-binding protein is conjugated to at least one molecular cargo (e.g., polynucleotide molecule, polypeptide molecule, or liposome or LNP).

In some embodiments, a CACNG1-binding protein is conjugated to the 5′ terminus of a polynucleotide molecule, the 3′ terminus of a polynucleotide molecule, an internal site on a polynucleotide molecule, or in any combinations thereof.

In some embodiments, a CACNG1-binding protein is conjugated to the N terminus of a polypeptide molecule, the C terminus of a polypeptide molecule, an internal site on a polypeptide molecule, or in any combinations thereof.

In some embodiments, the CACNG1-binding protein is conjugated to at least one molecular cargo (e.g., at least one polynucleotide molecule, polypeptide molecule and/or liposome or LNP). In some embodiments, the CACNG1-binding protein is conjugated to at least 2, 3, 4, 5, 6, 7, 8, 10, 12, 16, 20, 24, 30 or more molecular cargoes described herein (e.g., at least 2, 3, 4, 5, 6, 7, 8, 10, 12, 16, 20, 24, 30 or more polynucleotide molecules, polypeptide molecules, and/or liposomes or LNPs).

In some embodiments, a protein-drug conjugate described herein comprises an anti-CACNG1 antibody conjugated to one siRNA molecule. In some embodiments, a protein-drug conjugate described herein comprises an anti-CACNG1 antibody conjugated to two siRNA molecules.

In some embodiments, a protein-drug conjugate described herein comprises an anti-CACNG1 scFv conjugated to one siRNA molecule. In some embodiments, a protein-drug conjugate described herein comprises an anti-CACNG1 scFv conjugated to two siRNA molecules.

In some embodiments, a protein-drug conjugate described herein comprises an anti-CACNG1 Fab conjugated to one siRNA molecule. In some embodiments, a protein-drug conjugate described herein comprises an anti-CACNG1 Fab conjugated to two siRNA molecules.

In some embodiments, a protein-drug conjugate described herein comprises an anti-CACNG1 one-armed antibody conjugated to one siRNA molecule. In some embodiments, a protein-drug conjugate described herein comprises an anti-CACNG1 one-armed antibody conjugated to two siRNA molecules.

In some embodiments, the CACNG1-binding protein is conjugated to a molecular cargo (e.g., polynucleotide molecule, polypeptide molecule, or liposome or LNP) non-specifically. In some embodiments, the CACNG1-binding protein is conjugated to a molecular cargo (e.g., polynucleotide molecule, polypeptide molecule, or liposome or LNP) via a lysine residue or a cysteine residue, in a non-site-specific manner. In some embodiments, the CACNG1-binding protein is conjugated to a molecular cargo (e.g., polynucleotide molecule, polypeptide molecule, or liposome or LNP) via a lysine residue (e.g., lysine residue present in the CACNG1-binding protein) in a non-site-specific manner. In some cases, the CACNG1-binding protein is conjugated to a molecular cargo (e.g., polynucleotide molecule, or liposome or LNP) via a cysteine residue (e.g., cysteine residue present in the CACNG1-binding protein) in a non-site-specific manner.

In some embodiments, the CACNG1-binding protein is conjugated to a molecular cargo (e.g., polynucleotide molecule, polypeptide molecule, or liposome or LNP) in a site-specific manner. In some embodiments, the CACNG1-binding protein is conjugated to a molecular cargo (e.g., polynucleotide molecule, polypeptide molecule, or liposome or LNP) through a lysine residue, a cysteine residue, at the N-terminus, at the C-terminus, an unnatural amino acid, or an enzyme-modified or enzyme-catalyzed residue, via a site-specific manner. In some embodiments, the CACNG1-binding protein is conjugated to a molecular cargo (e.g., polynucleotide molecule, or liposome or LNP) through a lysine residue (e.g., lysine residue present in the CACNG1-binding protein) via a site-specific manner. In some embodiments, the CACNG1-binding protein is conjugated to a molecular cargo (e.g., polynucleotide molecule, polypeptide molecule, or liposome or LNP) through a cysteine residue (e.g., cysteine residue present in the CACNG1-binding protein) via a site-specific manner. In some embodiments, the CACNG1-binding protein is conjugated to a molecular cargo (e.g., polynucleotide molecule, polypeptide molecule, or liposome or LNP) at the N-terminus via a site-specific manner. In some embodiments, the CACNG1-binding protein is conjugated to a molecular cargo (e.g., polynucleotide molecule, polypeptide molecule, or liposome or LNP) at the C-terminus via a site-specific manner. In some embodiments, the CACNG1-binding protein is conjugated to a molecular cargo (e.g., polynucleotide molecule, polypeptide molecule, or liposome or LNP) through an unnatural amino acid via a site-specific manner. In some embodiments, the CACNG1-binding protein is conjugated to a molecular cargo (e.g., polynucleotide molecule, or liposome or LNP) through an enzyme-modified or enzyme-catalyzed residue via a site-specific manner.

In some embodiments, one or more molecular cargoes (e.g., polynucleotide molecule, polypeptide molecule, and/or liposome or LNP) is conjugated to a CACNG1-binding protein. In some embodiments, about 1, 2, 3, 4, 5, 6, 7, 8, 9, 10, 11, 12, 13, 14, 15, 16, 20, 24, 30, 36 or more molecular cargoes (e.g., polynucleotide molecule, polypeptide molecule, and/or liposome or LNP) are conjugated to one CACNG1-binding protein. In some embodiments, 1 molecular cargo is conjugated to one CACNG1-binding protein. In some embodiments, 2 molecular cargoes are conjugated to one CACNG1-binding protein. In some embodiments, 3 molecular cargoes are conjugated to one CACNG1-binding protein. In some embodiments, 4 molecular cargoes are conjugated to one CACNG1-binding protein. In some embodiments, 5 molecular cargoes are conjugated to one CACNG1-binding protein. In some embodiments, 6 molecular cargoes are conjugated to one CACNG1-binding protein. In some embodiments, 7 molecular cargoes are conjugated to one CACNG1-binding protein. In some embodiments, 8 molecular cargoes are conjugated to one CACNG1-binding protein. In some embodiments, 9 molecular cargoes are conjugated to one CACNG1-binding protein. In some embodiments, 10 molecular cargoes are conjugated to one CACNG1-binding protein. In some embodiments, 11 molecular cargoes are conjugated to one CACNG1-binding protein. In some embodiments, 12 molecular cargoes are conjugated to one CACNG1-binding protein. In some embodiments, 13 molecular cargoes are conjugated to one CACNG1-binding protein. In some embodiments, 14 molecular cargoes are conjugated to one CACNG1-binding protein. In some embodiments, 15 molecular cargoes are conjugated to one CACNG1-binding protein. In some embodiments, 16 molecular cargoes are conjugated to one CACNG1-binding protein. In some cases, the one or more molecular cargoes are the same. In other cases, the one or more molecular cargoes are different.

In some embodiments, the number of molecular cargoes conjugated to a CACNG1-binding protein forms a ratio. In some embodiments, the ratio is referred to as a DAR (drug-to-antibody) ratio, in which the drug as referred to herein is a molecular cargo described herein (e.g., polynucleotide molecule, polypeptide molecule, or liposome or LNP). In some embodiments, the DAR ratio of the molecular cargo to CACNG1-binding protein is about 1, 2, 3, 4, 5, 6, 7, 8, 9, 10, 11, 12, 13, 14, 15, 16, 20, 24, 30, 36 or greater. In some embodiments, the DAR ratio of the molecular cargo to CACNG1-binding protein is about 1 or greater. In some embodiments, the DAR ratio of the molecular cargo to CACNG1-binding protein is about 2 or greater. In some embodiments, the DAR ratio of the molecular cargo to CACNG1-binding protein is about 3 or greater. In some embodiments, the DAR ratio of the molecular cargo to CACNG1-binding protein is about 4 or greater. In some embodiments, the DAR ratio of the molecular cargo to CACNG1-binding protein is about 5 or greater. In some embodiments, the DAR ratio of the molecular cargo to CACNG1-binding protein is about 6 or greater. In some embodiments, the DAR ratio of the molecular cargo to CACNG1-binding protein is about 7 or greater. In some embodiments, the DAR ratio of the molecular cargo to CACNG1-binding protein is about 8 or greater. In some embodiments, the DAR ratio of the molecular cargo to CACNG1-binding protein is about 9 or greater. In some embodiments, the DAR ratio of the molecular cargo to CACNG1-binding protein is about 10 or greater. In some embodiments, the DAR ratio of the molecular cargo to CACNG1-binding protein is about 11 or greater. In some embodiments, the DAR ratio of the molecular cargo to CACNG1-binding protein is about 12 or greater. In some embodiments, the DAR ratio of the molecular cargo to CACNG1-binding protein is about 16 or greater. In some embodiments, the DAR ratio of the molecular cargo to CACNG1-binding protein is about 20 or greater. In some embodiments, the DAR ratio of the molecular cargo to CACNG1-binding protein is about 24 or greater.

In some embodiments, the DAR ratio of the molecular cargo to CACNG1-binding protein is about 1, 2, 3, 4, 5, 6, 7, 8, 9, 10, 11, 12, 13, 14, 15, 16, 20, 24, 30, or 36. In some embodiments, the DAR ratio of the molecular cargo to CACNG1-binding protein is about 1. In some embodiments, the DAR ratio of the molecular cargo to CACNG1-binding protein is about 2. In some embodiments, the DAR ratio of the molecular cargo to CACNG1-binding protein is about 3. In some embodiments, the DAR ratio of the molecular cargo to CACNG1-binding protein is about 4. In some embodiments, the DAR ratio of the molecular cargo to CACNG1-binding protein is about 5. In some embodiments, the DAR ratio of the molecular cargo to CACNG1-binding protein is about 6. In some embodiments, the DAR ratio of the molecular cargo to CACNG1-binding protein is about 7. In some embodiments, the DAR ratio of the molecular cargo to CACNG1-binding protein is about 8. In some embodiments, the DAR ratio of the molecular cargo to CACNG1-binding protein is about 9. In some embodiments, the DAR ratio of the molecular cargo to CACNG1-binding protein is about 10. In some embodiments, the DAR ratio of the molecular cargo to CACNG1-binding protein is about 11. In some embodiments, the DAR ratio of the molecular cargo to CACNG1-binding protein is about 12. In some embodiments, the DAR ratio of the molecular cargo to CACNG1-binding protein is about 13. In some embodiments, the DAR ratio of the molecular cargo to CACNG1-binding protein is about 14. In some embodiments, the DAR ratio of the molecular cargo to CACNG1-binding protein is about 15. In some embodiments, the DAR ratio of the molecular cargo to CACNG1-binding protein is about 16.

In some embodiments, liposome or LNP functionalization with binding moieties is carried out via the adsorption phenomenon, covalent-nature binding, or binding by the use of adapter molecules or linkers.

### Adsorption

This phenomenon is a non-covalent immobilization strategy that comprises physical adsorption and ionic binding. Physical adsorption occurs via weak interactions such as hydrogen bonding, electrostatic, hydrophobic and Van der Waals attractive forces, while ionic binding occurs between the opposite charges of the CACNG1-binding protein and liposome or LNP surfaces. However, when compared to other methodologies such as covalent binding, adsorption provides less stability. On the other hand, the fact that the interaction is non-covalent may allow easier release of the cargo in the tumor tissue.

### Covalent Strategies

Covalent binding requires prior activation of the LNPs. In some embodiments, covalent strategies occur via carbodiimide chemistry, maleimide chemistry or “click chemistry”, as discussed in detail below.

### Conjugation Chemistry

In some embodiments, a molecular cargo described herein (e.g., a polynucleotide molecule or polypeptide molecule described herein, or a liposome or LNP) is conjugated to a CACNG1-binding protein. In some embodiments, a molecular cargo described herein (e.g., a polynucleotide molecule or polypeptide molecule described herein, or a liposome or LNP) is conjugated to a CACNG1-binding protein directly. In some embodiments, a molecular cargo described herein (e.g., a polynucleotide molecule or polypeptide molecule described herein, or a liposome or LNP) is conjugated to a CACNG1-binding protein via a linker covalently connecting the CACNG1-binding protein with the molecular cargo. In some embodiments, the CACNG1-binding protein is an antibody or antigen binding fragment thereof (e.g., scFv or Fab).

In some embodiments, the molecular cargo described herein (e.g., a polynucleotide molecule or polypeptide molecule described herein, or a liposome or LNP) is conjugated to the CACNG1-binding protein by a chemical ligation process. In some embodiments, the molecular cargo described herein (e.g., a polynucleotide molecule or polypeptide molecule described herein, or a liposome or LNP) is conjugated to the CACNG1-binding protein by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910. In some embodiments, the molecular cargo described herein (e.g., a polynucleotide molecule or polypeptide molecule, described herein, or a liposome or LNP) is conjugated to the CACNG1-binding protein either site-specifically or non-specifically via native ligation chemistry.

In some embodiments, the molecular cargo described herein (e.g., a polynucleotide molecule or polypeptide molecule described herein, or a liposome or LNP) is conjugated to the CACNG1-binding protein by a site-directed method utilizing a “traceless” coupling technology (Philochem). In some embodiments, the “traceless” coupling technology utilizes an N-terminal 1,2-aminothiol group on the CACNG1-binding protein which is then conjugated with a molecular cargo described herein (e.g., a polynucleotide molecule or polypeptide molecule described herein, or a liposome or LNP) containing an aldehyde group. (see Casi et al., “Site-specific traceless coupling of potent cytotoxic drugs to recombinant antibodies for pharmacodelivery,” JACS 134(13): 5887-5892 (2012)).

In some embodiments, the molecular cargo described herein (e.g., a polynucleotide molecule or polypeptide molecule described herein, or a liposome or LNP) is conjugated to the CACNG1-binding protein by a site-directed method utilizing an unnatural amino acid incorporated into the CACNG1-binding protein. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some embodiments, the molecular cargo described herein (e.g., a polynucleotide molecule or polypeptide molecule described herein, or a liposome or LNP) is conjugated to the CACNG1-binding protein by a site-directed method utilizing an enzyme-catalyzed process. In some embodiments, the site-directed method utilizes SMARTag™ technology (Catalent, Inc.). In some embodiments, the SMARTag™ technology comprises generation of a formylglycine (FGIy) residue from cysteine by formylglycine-generating enzyme (FGE) through an oxidation process under the presence of an aldehyde tag and the subsequent conjugation of FGIy to an alkylhydraine-functionalized molecular cargo described herein (e.g., a polynucleotide molecule or polypeptide molecule described herein, or a liposome or LNP) via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some embodiments, the enzyme-catalyzed process comprises transglutaminase (TG), e.g., microbial transglutaminase (mTG). In some cases, the molecular cargo described herein (e.g., a polynucleotide molecule or polypeptide molecule described herein, or a liposome or LNP) is conjugated to the CACNG1-binding protein utilizing a microbial transglutaminase-catalyzed process. In some embodiments, mTG catalyzes the formation of a covalent bond between the amide side chain of a glutamine within the recognition sequence and a primary amine of a functionalized molecular cargo described herein (e.g., a polynucleotide molecule or polypeptide molecule described herein, or a liposome or LNP). In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some embodiments, a sequence of amino acids comprising an acceptor glutamine residue are incorporated into (e.g., appended to) a polypeptide sequence, under suitable conditions, for recognition by a TG. This sequence leads to cross-linking by the TG through a reaction between an amino acid side chain within the sequence of amino acids and a reaction partner. The recognition tag may be a peptide sequence that is not naturally present in the polypeptide comprising the TG recognition tag. In some embodiments, the TG recognition tag comprises at least one Gln. in some embodiments, the TGase recognition tag comprises an amino acid sequence XXQX, wherein X is any amino acid (e.g., conventional amino acid Leu, Ala, Gly, Ser, Val, Phe, Tyr, His, Arg, Asn, Glu, Asp, Cys, Gln, IIe, Met, Pro, Thr, Lys, or Trp or nonconventional amino acid). In some embodiments, the acyl donor glutamine-containing tag comprises an amino acid sequence selected from the group consisting of LLQGG (SEQ ID NO: 389), LLQG (SEQ ID NO: 390), LSLSQG (SEQ ID NO: 391), GGGLLQGG (SEQ ID NO: 392), GLLQG (SEQ ID NO: 393), LLQ, GSPLAQSHGG (SEQ ID NO: 395), GLLQGGG (SEQ ID NO: 396), GLLQGG (SEQ ID NO: 397), GLLQ (SEQ ID NO: 398), LLQLLQGA (SEQ ID NO: 399), LLQGA (SEQ ID NO: 400), LLQYQGA (SEQ ID NO: 401), LLQGSG (SEQ ID NO: 402), LLQYQG (SEQ ID NO: 403), LLQLLQG (SEQ ID NO: 404), SLLQG (SEQ ID NO: 405), LLQLQ (SEQ ID NO: 406), LLQLLQ (SEQ ID NO: 407), and LLQGR (SEQ ID NO: 408). See, e.g., PCT Publication No. WO2012/059882. In some embodiments, the acyl donor glutamine-containing tag is present at the N-terminus of the antigen-binding protein. In some embodiments, the acyl donor glutamine-containing tag is present at the C-terminus of the antigen-binding protein. In some embodiments, the acyl donor glutamine-containing tag is present both at the N-terminus and the C-terminus of the antigen-binding protein.

In some embodiments, the molecular cargo described herein (e.g., a polynucleotide molecule or polypeptide molecule described herein, or a liposome or LNP) is conjugated to the CACNG1-binding protein by a method as described in PCT Publication No. W02014/140317, which utilizes a sequence-specific transpeptidase. In some embodiments, the molecular cargo described herein (e.g., a polynucleotide molecule or polypeptide molecule described herein, or a liposome or LNP) is conjugated to the CACNG1-binding protein by a method as described in U.S. Patent Publication Nos. 2015/0105539 and 2015/0105540.

In some embodiments, the molecular cargo described herein (e.g., a polynucleotide molecule or polypeptide molecule described herein, or a liposome or LNP) is conjugated to the CACNG1-binding protein utilizing Azide-Alkyne Cycloaddition (CuAAC) click chemistry. Azides and alkynes can undergo catalyst free [3+2] cycloaddition by a using the reaction of activated alkynes with azides. Such catalyst-free [3+2] cycloaddition can be used in the methods described herein to conjugate a CACNG1-binding protein and the molecular cargo described herein (e.g., a polynucleotide molecule or polypeptide molecule described herein, or a liposome or LNP). Alkynes can be activated by ring strain such as, by way of example only, eight-membered ring structures, or nine-membered, appending electron-withdrawing groups to such alkyne rings, or alkynes can be activated by the addition of a Lewis acid such as, by way of example only, Au(l) or Au(III).

Alkynes activated by ring strain have been described and used in “copperless” [3+2] cycloaddition. For example, the cyclooctynes and difluorocyclooctynes described by Agard et al., J. Am. Chem. Soc., 126 (46):15046-15047 (2004), the dibenzocyclooctynes described by Boons et al., PCT International Publication No. WO 2009/067663 A1 (2009), the aza-dibenzocyclooctynes described by Debets et al., Chem. Comm., 46:97-99 (2010), and the cyclononynes described by Dommerholt et al., Angew. Chem. 122:9612-9615 (2010)). In some embodiments, a tetrazine (Tzn)-activated CACNG1-binding protein may be cross-linked to a trans-cyclooctene (TCO)-activated molecular cargo described herein (e.g., a polynucleotide molecule or polypeptide molecule described herein, or a liposome or LNP). In some embodiments, a TCO-activated CACNG1-binding protein may be crosslinked to a Tzn-activated molecular cargo described herein (e.g., a polynucleotide molecule or polypeptide molecule described herein, or a liposome or LNP).

### Linkers

Complexes described herein generally comprise a linker that connects a binding agent to a molecular cargo (e.g., a polynucleotide molecule, polypeptide molecule, a liposome, or an LNP). A linker comprises at least one covalent bond. In some embodiments, a linker may be a single bond, e.g., a disulfide bond or disulfide bridge, that connects a binding agent to a polynucleotide molecule, polypeptide molecule, or a liposome or LNP. However, in some embodiments, a linker may connect a binding agent to a polynucleotide molecule, polypeptide molecule, or a liposome or LNP through multiple covalent bonds. A linker is generally stable in vitro and in vivo, and may be stable in certain cellular environments. Additionally, generally a linker does not negatively impact the functional properties of either the binding agent or the polynucleotide molecule, polypeptide molecule, or a liposome or LNP. Examples and methods of synthesis of linkers are known in the art (see, e.g. Kline, T. et al. “Methods to Make Homogenous Antibody Drug Conjugates.” Pharmaceutical Research, 2015, 32:11, 3480-3493; Jain, N. et al. “Current ΔΔCt C Linker Chemistry” Pharm Res. 2015, 32:11, 3526-3540; McCombs, J. R. and Owen, S. C., “Antibody Drug Conjugates: Design and Selection of Linker, Payload and Conjugation Chemistry” AAPS J. 2015, 17:2, 339-351).

A precursor to a linker typically will contain two different reactive species that allow for attachment to both the binding agent and a polynucleotide molecule, polypeptide molecule, or a liposome or LNP. In some embodiments, the two different reactive species may be a nucleophile and/or an electrophile. In some embodiments, a linker is connected to a binding agent via conjugation to a lysine residue or a cysteine residue of the binding agent. In some embodiments, a linker is connected to a cysteine residue of a muscle-targeting agent via a maleimide-containing linker, wherein optionally the maleimide-containing linker comprises a maleimidocaproyl or maleimidomethyl cyclohexane-1-carboxylate group. In some embodiments, a linker is connected to a cysteine residue of a muscle-targeting agent or thiol functionalized molecular cargo via a 3-arylpropionitrile functional group. In some embodiments, a linker is connected to a binding agent and/or a polynucleotide molecule, polypeptide molecule, or an LNP via an amide bond, a hydrazide, a triazole, a thioether or a disulfide bond.

In some embodiments, a linker described herein is a cleavable linker or a non-cleavable linker. In some embodiments, the linker is a cleavable linker. In other embodiments, the linker is a non-cleavable linker.

### Cleavable Linkers

A cleavable linker may be a protease-sensitive linker, a pH-sensitive linker, or a glutathione-sensitive linker. These linkers are generally cleavable only intracellularly and are preferably stable in extracellular environments.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by an disulfide exchange reaction with a glutathione species inside a cell. In some embodiments, the disulfide moiety further comprises at least one amino acid, e.g. a cysteine residue.

### Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyneazide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation will be utilized to covalently link a muscle-targeting agent comprising a LPXT sequence to a molecular cargo comprising a (G), sequence (see, e.g. Proft T. Sortase-mediated protein ligation: an emerging biotechnology tool for protein modification and immobilization. Biotechnol Lett. 2010, 32(1):1-10).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species, an optionally substituted sulfur species, or a poly(alkylene oxide), e.g. polyethylene oxide or polypropylene oxide.

In some cases, the linker is a non-polymeric linker. A non-polymeric linker refers to a linker that does not contain a repeating unit of monomers generated by a polymerization process. Exemplary non-polymeric linkers include, but are not limited to, C1-C30 alkyl group (e.g., a C5, C4, C 3, C 2, or C1 alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C1-C30 alkyl group (e.g., a C5, C4, C 3, C 2, or C1 alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups. In one embodiment, the linker has a structure

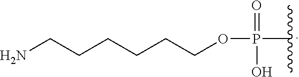 [SMILES: *P(=O)(O)OCCCCCCN]

In some cases, the non-polymeric linker does not encompass a polyalkylene oxide (e.g., PEG). In some cases, the non-polymeric linker does not encompass a PEG.

In some embodiments, the linker comprises a homobifunctional linker. Exemplary homobifunctional linkers include, but are not limited to, organoazide, organoalkyne, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido) butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-113-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, a,a′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio) propionate (sPDP), long-chain N-succinimidyl 3-(2-pyridyldithio) propionate (LC-sPDP), water-soluble-long-chain N-succinimidyl 3-(2-pyridyldithio) propionate (sulfo-LCsPDP), succinimidyloxycarbonyl-a-methyl-a-(2-pyridyldithio) toluene (sMPT), sulfosuccinimidyl-6-[a-methyl-a-(2-pyridyldithio)toluamido]hexanoate (sulfo-LC-sMPT), succinimidyl-4-(N-maleimidomethyl) cyclohexane-1-car-boxylate (sMCC), sulfosuccinimidyl-4-(N-maleimidomethyl) cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MB s), N-succinimidyl (4-iodoacteyl)aminobenzoate (sIAB), sulfosuccinimidyl (4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(y-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino) hexanoyl)amino]hexanoate (slAXX), succinimidyl 4-(((iodoacetyl) amino)methyl)cyclohexane-I-carboxylate (slAC), succinimidyl 6-((((4-iodoacetyl)amino)methyl)cyclohexane-1-carbonyl)amino) hexanoate (slACX), p-nitrophenyl iodoacetate (NPIA), carbonyl-reactive and sulfhydrylreactive cross-linkers such as 4-(4-N-maleimidophenyl) butyric acid hydrazide (MPBH), 4-(N-maleimidomethyl) cyclohexane-I-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido2′-nitrophenylamino)hexanoate (sANPAH), sulfo succinimidyl-6-(4′-azido-2′-nitrophenylamino)hexanoate(sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidopheny1)_{1,3}′-dithiopropionate (sΔΔCt P), N-sulfosuccinimidyl(4-azidophenyl)-1,3′-dithiopropionate (sulfo-sΔΔCt P), sulfosuccinimidyl 4-(p-azidophenyl) butyrate (sulfo-sAPB), sulfosuccinimidyl 2-(7-azido-4-methylcoumarin-3-acetamide)ethy1-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive crosslinkers such as 1-(p-Azidosalicylamido)-4-(iodoacetamido) butane (AslB), N-[4-(p-azidosalicylamido)buty1]-3′-(2′-pyridyldithio)propionamide (APDP), benzophenone-4-iodoacetamide, benzophenone-4-maleimide carbonylreactive and photoreactive cross-linkers such as p-azidobenzoyl hydrazide (ABH), carboxylate-reactive and photoreactive cross-linkers such as 4-(p-azidosalicylamido) butylamine (AsBA), and arginine-reactive and photoreactive cross-linkers such as p-azidophenyl glyoxal (APG).

In some embodiments, the linker comprises a reactive functional group. In some cases, the reactive functional group comprises a nucleophilic group that is reactive to an electrophilic group present on a CACNG1-binding protein. Exemplary electrophilic groups include carbonyl groups such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (mc). In some cases, the linker comprises maleimidocaproyl (mc). In some cases, the linker is maleimidocaproyl (mc). In other embodiments, the maleimide group comprises a maleimidomethyl group, such as succinimidy1-4-(N-maleimidomethyl)cyclohexane-I-carboxylate (sMCC) or sulfosuccinimidy1-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some embodiments, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some embodiments, the linker comprises a self-stabilizing maleimide. In some embodiments, the linker is a self-stabilizing maleimide.

In some embodiments, the linker comprises at least one azide moiety, e.g., as part of an organoazide moiety. In some embodiments, the linker comprises at least one alkyne moiety, e.g., as part of an organoalkyne moiety. In one embodiment, the alkyne is an activated alkyne. In some embodiments, the linker comprises a trizole (e.g., formed via a 1,3-cycloaddition reaction of an azide and an alkyne). In some embodiments, the linker comprises a Diels-Alder adduct.

In some embodiments, the linker comprises a peptide moiety. In some embodiments, the peptide moiety comprises at least 2, 3, 4, 5, or 6 more amino acid residues. In some embodiments, the peptide moiety comprises at most 2, 3, 4, 5, 6, 7, or 8 amino acid residues. In some embodiments, the peptide moiety comprises about 2, about 3, about 4, about 5, or about 6 amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly (SEQ ID NO: 425), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 426), or Gly-Phe-Leu-Gly (SEQ ID NO: 427). In some embodiments, the linker comprises a peptide moiety such as: Val-Cit (valine-citrulline), Gly-Gly-Phe-Gly (SEQ ID NO: 425), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 426), or Gly-Phe-Leu-Gly (SEQ ID NO: 427). In some cases, the linker comprises Val-Cit. In some cases, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (mc). In some embodiments, the peptide group is val-cit. In some embodiments, the benzoic acid group is PABA. In some embodiments, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO 2015/038426.

In some embodiments, the linker is a dendritic type linker. In some embodiments, the dendritic type linker comprises a branching, multifunctional linker moiety. In some embodiments, the dendritic type linker is used to increase the molar ratio of polynucleotide B to the CACNG1-binding protein. In some embodiments, the dendritic type linker comprises PAMAM dendrimers. In some embodiments, the dendritic type linker comprises triazoles. In some embodiments, the triazoles are connected by PEG links. In some embodiments, the linkers are as described in WO 2022/015656.

In some embodiments, the linker is a traceless linker or a linker in which after cleavage does not leave behind a linker moiety (e.g., an atom or a linker group) to a CACNG1-binding protein or a polynucleotide B. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., “Traceless solid-phase organic synthesis,” Chem. Rev. 102: 2607-2024 (2002). In some embodiments, a linker is a traceless linker as described in U.S. Pat. No. 6,821,783.

In some embodiments, the linker is a linker described in U.S. Pat. Nos. 6,884,869; 7,498,298; 8,288,352; 8,609,105; or 8,697,688; U.S. Patent Publication Nos. US2014/0127239; US2013/028919; US2014/286970; US2013/0309256; US2015/037360; and US2014/0294851; or International Application Publication Nos. WO2015/057699; WO2014/080251; WO2014/197854; WO2014/145090; WO2014/177042, WO2022/015656.

In some embodiments, a linker is a bond, i.e., a linker is absent. In some cases, a linker is a non-polymeric linker. In some cases, a linker is a polymeric linker.

In some embodiments, the linker comprises an alkyl group. In some embodiments, the linker comprises a C1-C30 alkyl group, or a C1-C24 alkyl group, or a C1-C20 alkyl group, or a C1-C16 alkyl group, or a C1-C12 alkyl group, or a C1-C10 alkyl group, or a C1-C8 alkyl group, or a C1-C6 alkyl group, or a C1-C4 alkyl group. In some cases, a linker is a C1-C6 alkyl group, such as for example, a C 3, C 4, C 3, C 2, or C1 alkyl group. In some cases, the C1-C6 alkyl group is an unsubstituted C1-C6 alkyl group. As used in the context of a linker, alkyl means a saturated straight or branched hydrocarbon radical containing up to six carbon atoms. In some embodiments, the linker comprises a homobifunctional linker or a heterobifunctional linker described supra.

In some cases, a linker is an oligomeric or a polymeric linker. In some embodiments, a linker is a natural or synthetic oligomer or polymer, consisting of branched or unbranched monomers, and/or cross-linked network of monomers in two or three dimensions. In some embodiments, the linker comprises a polysaccharide, lignin, rubber, or polyalkylen oxide (e.g., polyethylene glycol).

In some embodiments, the at least one polymeric linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylene terephthalate (also known as poly(ethylene terephthalate), PET, PETG, or PETE), polytetramethylene glycol (PTG), or polyurethane as well as mixtures thereof. In some embodiments, the linker comprises polyalkylene oxide. In some embodiments, the linker comprises PEG. In some embodiments, the linker comprises polyethylene imide (PEI) or hydroxy ethyl starch (HES).

In some embodiments, the linker comprises a polyalkylene oxide (e.g., PEG) comprising discrete ethylene oxide units. In some cases, the linker comprises between about 2 and about 48 ethylene oxide units. In some cases, the polymer moiety C comprises about 2, about 3, about 4, about 5, about 6, about 7, about 8, about 9, about 10, about 11, about 12, about 13, about 14, about 15, about 16, about 17, about 18, about 19, about 20, about 24, about 30, about 36, about 42, or about 48 ethylene oxide units.

In some embodiments, the CACNG1-binding protein is conjugated to the molecular cargo described herein (e.g., a polynucleotide molecule, polypeptide molecule described herein, or a liposome or LNP) using a protamine linker, as disclosed in the U.S. Patent Application Publication Nos. US2002/0132990, US2004/0023902, US2007/012152, and US2010/0209440. In some embodiments, a protamine linker encompassed for use in the present disclosure comprises a sequence disclosed in US 2010/0209440.

Acid cleavable linkers can also be used with the present disclosure and include, but are not limited to, bismaleimideothoxy propane, adipic acid dihydrazide linkers (see, e.g., Fattom et al., Infection & Immun. 60:584 589, 1992) and acid labile transferrin conjugates that contain a sufficient portion of transferrin to permit entry into the intracellular transferrin cycling pathway (see, e.g., Welhoner et al., J. Biol. Chem. 266:4309 4314, 1991). Conjugates linked via acid cleavable linkers should be preferentially cleaved in acidic intracellular compartments, such as the endosome.

Photocleavable linkers can also be used with the present disclosure. Photocleavable linkers are cleaved upon exposure to light (see, e.g., Goldmacher et al., Bioconj. Chem. 3:104-107, 1992), thereby releasing the targeted agent upon exposure to light. (Hazum et al., Proc. Eur. Pept. Symp., 16th, Brunfeldt, K (Ed), pp. 105 110, 1981; nitrobenzyl group as a photocleavable protective group for cysteine; Yen et al., Makromol. Chem 190:69 82, 1989; water soluble photocleavable copolymers, including hydroxypropylmethacrylamide copolymer, glycine copolymer, fluorescein copolymer and methylrhodamine copolymer; and Senter et al., Photochem. Photobiol. 42:231 237, 1985; nitrobenzyloxy carbonyl chloride cross linking reagents that produce photocleavable linkages), relevant portions incorporated herein by reference. Such linkers are particularly useful in treating dermatological or ophthalmic conditions. In addition, other tissues, such as blood vessels that can be exposed to light using fiber-optics during angioplasty in the prevention or treatment of restenosis may benefit from the use of photocleavable linkers. After administration of the conjugate, the body part is exposed to light, resulting in release of the targeted moiety from the conjugate. Heat sensitive linkers would also have similar applicability.

In one embodiment, the linker has a structure

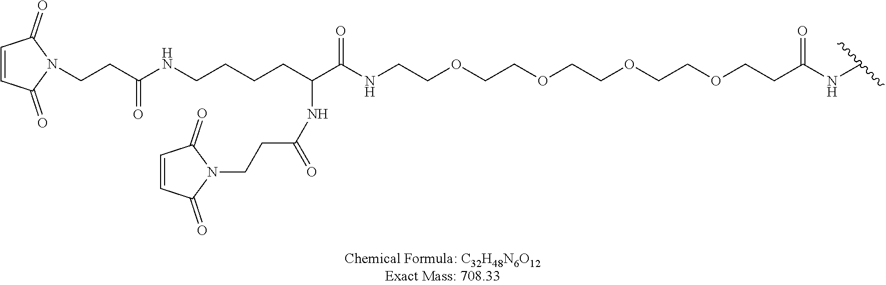 [SMILES: *NC(=O)CCOCCOCCOCCOCCNC(=O)C(CCCCNC(=O)CCN1C(=O)C=CC1=O)NC(=O)CCN1C(=O)C=CC1=O]

In certain embodiments provided herein, suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference. Generally, suitable binding agent linkers for the antibody conjugates described herein are those that are sufficiently stable to exploit the circulating half-life of the antibody and, at the same time, capable of releasing its payload after antigen-mediated internalization of the conjugate. Linkers can be cleavable or non-cleavable. Cleavable linkers include linkers that are cleaved by intracellular metabolism following internalization, e.g., cleavage via hydrolysis, reduction, or enzymatic reaction. Non-cleavable linkers include linkers that release an attached payload via lysosomal degradation of the antibody following internalization. Suitable linkers include, but are not limited to, acid-labile linkers, hydrolysis-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citrulline units, and para-aminobenzyl (PAB) units.

Any linker molecule or linker technology known in the art can be used to create or construct an ΔΔCt C of the present disclosure. In certain embodiments, the linker is a cleavable linker. According to other embodiments, the linker is a non-cleavable linker. Exemplary linkers that can be used in the context of the present disclosure include, linkers that comprise or consist of e.g., MC (6-maleimidocaproyl), MP (maleimidopropanoyl), val-cit (valine-citrulline), val-ala (valine-alanine), val-gly (valine-glycine), dipeptide site in protease-cleavable linker, ala-phe (alanine-phenylalanine), dipeptide site in protease-cleavable linker, PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate), and variants and combinations thereof. Additional examples of linkers that can be used in the context of the present disclosure are provided, e.g., in U.S. Pat. No. 7,754,681 and in Ducry, Bioconjugate Chem., 2010, 21:5-13, and the references cited therein, the contents of which are incorporated by reference herein in their entireties.

In certain embodiments, the linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the linker comprises a non-cleavable moiety.

Suitable linkers also include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the linker comprises one or more amino acids. Suitable amino acids include natural, non-natural, standard, non-standard, proteinogenic, non-proteinogenic, and L- or D-a-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the linker comprises lysine, valine, and citrulline. In some embodiments, the linker comprises lysine, valine, and alanine. In some embodiments, the linker comprises valine and alanine.

In some embodiments, the linker comprises a self-immolative group. The self-immolative group can be any such group known to those of skill. In particular embodiments, the self-immolative group is p-aminobenzyl (PAB), or a derivative thereof. Useful derivatives include p-aminobenzyloxycarbonyl (PABC). Those of skill will recognize that a self-immolative group is capable of carrying out a chemical reaction which releases the remaining atoms of a linker from a payload.

In some embodiments, the linker is:

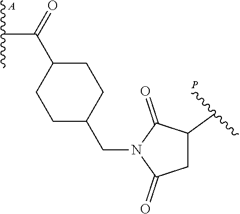

wherein

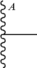

is a bond to the antibody or antigen-binding protein (e.g., via lysine residue)
and

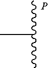

is a bond to the therapeutic payload (e.g., dihydrotestosterone (DHT) or testosterone, or a biologically equivalent variant thereof). In some embodiments, the linker is:

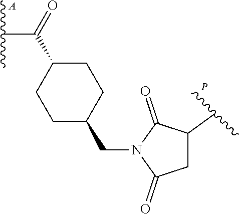

wherein

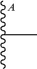

is a bond to the antibody or antigen-binding protein (e.g., via lysine residue)
and

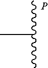

is a bond to a therapeutic payload (e.g., dihydrotestosterone (DHT) or testosterone, or a biologically equivalent variant thereof). In certain embodiments, the linker is:

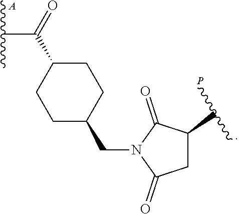

In certain embodiments, the linker is:

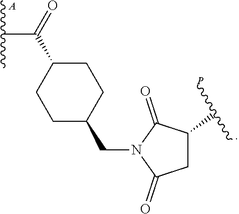

In some embodiments, the linker is derived from maleimidylmethyl-4-trans-cyclohexanecarboxysuccinate:

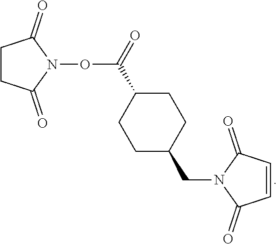 [SMILES: O=C1C=CC(=O)N1C[C@H]1CC[C@H](C(=O)ON2C(=O)CCC2=O)CC1]

In some embodiments, the linker is:

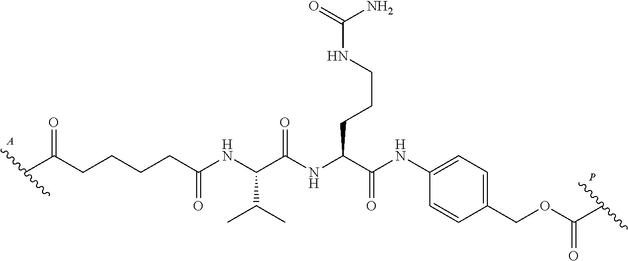

wherein

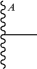

is a bond to the antibody or antigen-binding protein (e.g., via lysine residue)
and

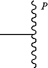

is a bond to therapeutic payload (e.g., dihydrotestosterone (DHT) or testosterone, or a biologically equivalent variant thereof).

In some embodiments, the linker is:

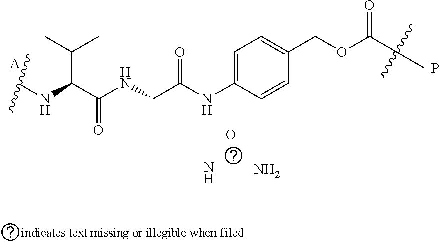

wherein

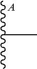

is a bond to the antibody or antigen-binding protein (e.g., via lysine residue)
and

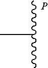

is a bond to therapeutic payload (e.g., dihydrotestosterone (DHT) or testosterone, or a biologically equivalent variant thereof).

The present disclosure comprises ADCs in which a linker connects an anti-hCACNG1 antigen-binding protein as described herein to therapeutic agent through an attachment at a particular amino acid within the antibody or antigen-binding molecule. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; and US 2013/0101546), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36(Suppl 1): 100). Site specific conjugation techniques, include, but are not limited to glutamine conjugation via transglutaminase (see e.g., Schibli, Angew Chemie Inter Ed. 2010, 49,9995). In some embodiments, a residue of an antibody as described herein, e.g., a residue in a heavy chain constant region of the antibody, may be substituted with a glutamine to further facilitate glutamine conjugation via transglutaminase. As a non-limiting example, a human heavy chain constant region may be modified with the N297Q substitution found in the sequence of the human IgG1 heavy chain constant region. Such substitution provides for a total of 4 glutamines for conjugation by transglutaminase.

The antibody drug conjugates described herein can be prepared using conjugation conditions known to those of ordinary skill in the art, (see, e.g., Doronina et al. Nature Biotechnology 2003, 21, 7, 778, which is incorporated herein by reference in its entirety). In some embodiments an anti-hCACNG1 antigen-binding protein drug conjugate is prepared by contacting an anti-hCACNG1 antigen-binding protein AS described herein with a compound comprising the desired linker and therapeutic agent, wherein said linker possesses a moiety that is reactive with the antibody or antigen-binding protein, e.g., at the desired residue of the antibody or antigen-binding protein.

In some embodiments, provided herein are processes for preparing an antibody-drug conjugate comprising contacting an anti-hCACNG1 antigen-binding protein described herein, including an azido-functionalized anti-hCACNG1 antigen binding protein, with a compound having the following formula A1:

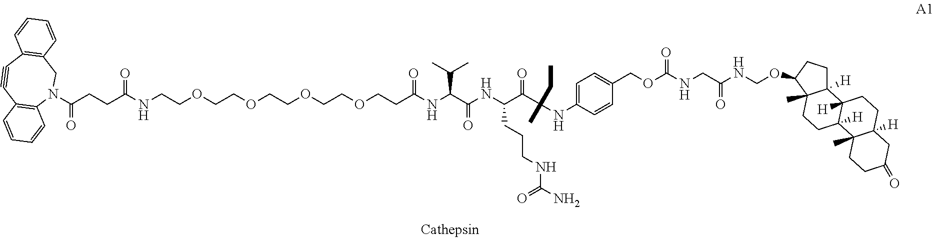

and a transglutaminase, see, e.g., U.S. Pat. No. 9,676,871, which is incorporated herein in its entirety by reference. Shown in A¹ is a cathepsin cleavage site.

### Polynucleotides and Methods of Making

The present disclosure includes any polynucleotide described herein, for example, encoding an immunoglobulin V_{H}, V_{L}, CDR-H, CDR-L, HC or LC disclosed herein, optionally, which is operably linked to a promoter or other expression control sequence. For example, the present disclosure provides any polynucleotide (e.g., DNA) that includes a nucleotide sequence set forth in SEQ ID NO: 181-360. In an embodiment, a polynucleotide of the present disclosure is fused to a secretion signal sequence. Polypeptides encoded by such polynucleotides are also within the scope of the present disclosure. A polynucleotide described herein can be DNA or RNA.

Nucleotide sequences of HCVRs and LCVRs of anti-CACNG1 protein-drug conjugates set forth herein are summarized below in Table 1-2. Polynucleotides encoding an anti-CACNG1 protein-drug conjugates, or polypeptide portion(s) thereof, that include one or more of the HCVRs and/or LCVRs set forth in Table 1-2 form part of the present disclosure.

TABLE 1-2
SEQ ID NOs or Sequences of Nucleotide Sequences encoding Domains in
Antibodies, Antigen-binding Fragments (e.g., Fabs or scFv Molecules) in Protein-
drug Conjugates of the Present Disclosure.

| | anti-CACNG1 | | | | | | | | | | |
|---|---|---|---|---|---|---|---|---|---|---|---|
| # | Molecule | HCVR | HCDR1 | HCDR2 | HCDR3 | LCVR | LCDR1 | LCDR2 | LCDR3 | HC | LC |
| 1 | H2aM31929N/ | 181 | 182 | 183 | 184 | 185 | 186 | GCT | 188 | 189 | 190 |
| | REGN10728 | | | | | | | GCA | | | |
| | | | | | | | | TCC | | | |
| 2 | H2aM31944N | 191 | 192 | 193 | 194 | 195 | 196 | GCT | 198 | 199 | 200 |
| | | | | | | | | GCA | | | |
| | | | | | | | | TCC | | | |
| 3 | H4H31265P2/ | 201 | 202 | 203 | 204 | 205 | 206 | GCT | 208 | 209 | 210 |
| | REGN5972 | | | | | | | GCA | | | |
| | | | | | | | | TCC | | | |
| 4 | H2aM31941N | 211 | 212 | 213 | 214 | 215 | 216 | GGT | 218 | 219 | 220 |
| | | | | | | | | GCA | | | |
| | | | | | | | | TCC | | | |
| 5 | REGN7660 | 221 | 222 | 223 | 224 | 225 | 226 | GGT | 228 | 229 | 230 |
| | | | | | | | | GCA | | | |
| | | | | | | | | TCC | | | |
| 6 | REGN9909 | 231 | 232 | 233 | 234 | 235 | 236 | AGG | 238 | 239 | 240 |
| | | | | | | | | AATA | | | |
| | | | | | | | | AT | | | |
| 7 | REGN10713 | 241 | 242 | 243 | 244 | 245 | 246 | GAC | 248 | 249 | 250 |
| | | | | | | | | AATA | | | |
| | | | | | | | | AT | | | |
| 8 | REGN10715 | 251 | 252 | 253 | 254 | 255 | 256 | GCT | 258 | 259 | 260 |
| | | | | | | | | ACA | | | |
| | | | | | | | | TCC | | | |
| 9 | REGN10716 | 261 | 262 | 263 | 264 | 265 | 266 | GGT | 268 | 269 | 270 |
| | | | | | | | | GCA | | | |
| | | | | | | | | TCC | | | |
| 10 | REGN10717 | 271 | 272 | 273 | 274 | 275 | 276 | AAG | 278 | 279 | 280 |
| | | | | | | | | GCG | | | |
| | | | | | | | | TCT | | | |
| 11 | REGN10783 | 281 | 282 | 283 | 284 | 285 | 286 | GCT | 288 | 289 | 290 |
| | | | | | | | | GCA | | | |
| | | | | | | | | TCC | | | |
| 12 | REGN7854 | 291 | 292 | 293 | 294 | 295 | 296 | GGT | 298 | 299 | 300 |
| | | | | | | | | GCA | | | |
| | | | | | | | | TCC | | | |
| 13 | REGN14570 | 301 | 302 | 303 | 304 | 305 | 306 | GCT | 308 | 309 | 310 |
| | | | | | | | | ACA | | | |
| | | | | | | | | TCC | | | |
| 14 | REGN14571 | 311 | 312 | 313 | 314 | 315 | 316 | GGT | 318 | 319 | 320 |
| | | | | | | | | GCA | | | |
| | | | | | | | | TCC | | | |
| 15 | REGN14572 | 321 | 322 | 323 | 324 | 325 | 326 | AAG | 328 | 329 | 330 |
| | | | | | | | | GCG | | | |
| | | | | | | | | TCT | | | |
| 16 | REGN14573 | 331 | 332 | 333 | 334 | 335 | 336 | GAC | 338 | 339 | 340 |
| | | | | | | | | AATA | | | |
| | | | | | | | | AT | | | |
| 17 | REGN14574 | 341 | 342 | 343 | 344 | 345 | 346 | GCT | 348 | 349 | 350 |
| | | | | | | | | GCA | | | |
| | | | | | | | | TCC | | | |
| 18 | REGN14647 | 351 | 352 | 353 | 354 | 355 | 356 | GCT | 358 | 359 | 360 |
| | | | | | | | | GCA | | | |
| | | | | | | | | TCC | | | |
| 19 | REGN10712 | 428 | 430 | 432 | 434 | 436 | 438 | TATA | 440 | 442 | 446 |
| | | | | | | | | ACA | | | |
| | | | | | | | | GC | | | |
| 20 | REGN9908 | 450 | 452 | 454 | 456 | 458 | 460 | AGA | 462 | 464 | 468 |
| | | | | | | | | AATA | | | |
| | | | | | | | | AT | | | |

In some embodiments, a nucleic acid molecule as described herein comprises a nucleic acid sequence encoding any of the HCVR amino acid sequences listed in Table 1-2; in certain embodiments the nucleic acid molecule comprises a polynucleotide sequence selected from any of the HCVR nucleic acid sequences listed in Table 1-2, or a substantially similar sequence thereof having at least 90%, at least 95%, at least 98% or at least 99% sequence identity thereto.

In some embodiments, the nucleic acid molecule comprises a polynucleotide sequence selected from any of the LCVR nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the HCDR1 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the HCDR2 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the HCDR3 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the LCDR1 nucleic acid sequences listed in Table 1-2, or a substantially similar sequence thereof having at least 90%, at least 95%, at least 98% or at least 99% sequence identity thereto.

In some embodiments the nucleic acid molecule comprises a polynucleotide sequence selected from any of the LCDR2 nucleic acid sequences listed in Table 1-2, or a substantially similar sequence thereof having at least 90%, at least 95%, at least 98% or at least 99% sequence identity thereto.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the disclosure. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ΔΔCt C (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present disclosure includes polynucleotides encoding immunoglobulin polypeptide chains which are variants nucleotide sequences. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear). In an embodiment, a variant of a nucleotide sequence comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment, be missense or nonsense mutations. In an embodiment, such a variant polynucleotide encodes an immunoglobulin polypeptide chain which can be incorporated into a CACNG1-binding protein, i.e., such that the protein retains specific binding to CACNG1.

Eukaryotic and prokaryotic host cells, including mammalian cells, may be used as hosts for expression of a CACNG1-binding protein (e.g., antibody or antigen-binding fragment thereof). Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present disclosure includes an isolated host cell (e.g., a CHO cell or any type of host cell set forth above) comprising an antigen-binding protein, a V_{H}, V_{L}, HC, LC or CDRs thereof (or variant thereof), disclosed herein; and/or a polynucleotide encoding one or more immunoglobulin chains thereof (e.g., as discussed herein).

The present disclosure also includes a cell which is expressing CACNG1 or an antigenic fragment or fusion thereof which is bound by an antigen-binding protein of the present disclosure (e.g., an antibody or antigen-binding fragment thereof), for example, wherein the cell is in the body of a subject or is in vitro. In addition, the present disclosure also provides a complex comprising a CACNG1-binding protein, e.g., antibody or antigen-binding fragment thereof, as discussed herein complexed with CACNG1 polypeptide or an antigenic fragment thereof or fusion thereof and/or with a secondary antibody or antigen-binding fragment thereof (e.g., detectably labeled secondary antibody) that binds specifically to the anti-CACNG1 antibody or fragment. In an embodiment of the disclosure, the complex is in vitro (e.g., is immobilized to a solid substrate) or is in the body of a subject.

In an embodiment, a myc tag has the amino acid sequence EQKLISEEDLGG (SEQ ID NO: 409), a His₆ (SEQ ID NO: 365) or hexahis (SEQ ID NO: 365) or hexahistidine tag (SEQ ID NO: 365) has the amino acid sequence HHHHHH (SEQ ID NO: 365), an mmh tag has the amino acid sequence EQKLISEEDLGGEQKLISEEDLHHHHHH (SEQ ID NO: 380) and a mouse Fc tag has the amino acid sequence EPRGPTIKPCPPCKCPAPNLLGGPSVFIFPPKIKDVLMISLSPIVTCVVVDVSEDDPDVQISWFVNNVEVHTAQTQT HREDYNSTLRVVSALPIQHQDWMSGKEFKCKVNNKDLPAPIERTISKPKGSVRAPQVYVLPPPEEEMTKKQVTLTCM VTDFMPEDIYVEWTNNGKTELNYKNTEPVLDSDGSYFMYSKLRVEKKNWVERNSYSCSVVHEGLHNHHTTKSFSRTP GK (SEQ ID NO: 410).

In addition, the present disclosure also provides a complex comprising an anti-CACNG1 protein-drug conjugate, as discussed herein complexed with a CACNG1 polypeptide or an antigenic fragment thereof or fusion thereof and/or with a secondary antibody or antigen-binding fragment thereof (e.g., detectably labeled secondary antibody) that binds specifically to the anti-CACNG1 protein-drug conjugate. In an embodiment, the complex is in vitro (e.g., is immobilized to a solid substrate) or is in the body of a subject.

Recombinant CACNG1-binding proteins, e.g., antibodies and antigen-binding fragments, or anti-CACNG1 fusion proteins disclosed herein may also be produced in an E. coli/T7 expression system. In this embodiment, polynucleotides encoding the anti-CACNG1 antibody immunoglobulin molecules described herein (e.g., HC, LC, V_{H} and/or V_{L} or CDRs thereof described herein) may be inserted into a pET-based plasmid and expressed in the E. coli/T7 system. For example, the present disclosure includes methods for expressing an antibody or antigen-binding fragment thereof or immunoglobulin chain thereof in a host cell (e.g., bacterial host cell such as E. coli such as BL21 or BL21 DE3) comprising expressing T7 RNA polymerase in the cell which also includes a polynucleotide encoding an immunoglobulin chain that is operably linked to a T7 promoter. For example, in an embodiment, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5; 189(1): 113-30.

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455. Thus, the present disclosure includes recombinant methods for making an anti-CACNG1 antigen-binding protein, such as an antibody or antigen-binding fragment thereof of the present disclosure, or an immunoglobulin chain thereof, comprising (i) introducing, into a host cell, one or more polynucleotides (e.g., including the nucleotide sequence in any one or more of SEQ ID NOs: 189, 199, 209, 219, 229, 239, 249, 259, 269, 279, 289, 299, 309, 319, 329, 339, 349, 359, 190, 200, 210, 220, 230, 240, 250, 260, 270, 280, 290, 300, 310, 320, 330, 340, 350, 360, or a variant thereof) encoding light and/or heavy immunoglobulin chains of the antigen-binding protein, for example, wherein the polynucleotide is in a vector; and/or integrates into the host cell chromosome and/or is operably linked to a promoter; (ii) culturing the host cell (e.g., CHO or Pichia or Pichia pastoris) under conditions favorable for expression of the polynucleotide and, (iii) optionally, isolating the antigen-binding protein (e.g., antibody or antigen-binding fragment) or chain from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment). The methods of the present disclosure include those wherein only a heavy immunoglobulin chain or only a light immunoglobulin chain or both (e.g., any of those discussed herein including mature fragments and/or variable domains thereof) are expressed in a cell. Such single chains are useful, for example, as intermediates in the expression of an antibody or antigen-binding fragment that includes such a chain. For example, the present disclosure also includes CACNG1-binding proteins, such as antibodies and antigen-binding fragments thereof which are the product of the production methods set forth herein, and, optionally, the purification methods set forth herein.

In an embodiment of the disclosure, a method for making a CACNG1-binding protein, e.g., antibody or antigen-binding fragment thereof, includes a method of purifying the antigen-binding protein, e.g., by column chromatography, precipitation and/or filtration. As discussed, the product of such a method also forms part of the present disclosure.

### Preparation of Human Antibodies

The anti-CACNG1 antibodies and antigen-binding fragments described herein can be fully human antibodies and fragments. Methods for generating monoclonal antibodies, including fully human monoclonal antibodies are known in the art. Any such known methods can be used in the context of the present disclosure to make human antibodies that specifically bind to CACNG1.

Antigen-binding domains specific for particular antigens can be prepared by any antibody generating technology known in the art. Once obtained, two different antigen-binding domains, specific for two different antigens (e.g., CACNG1 and a target antigen), can be appropriately arranged relative to one another to produce a bispecific antigen-binding molecule as described herein using routine methods. (A discussion of exemplary bispecific antibody formats that can be used to construct the bispecific antigen-binding molecules as described herein is provided elsewhere herein). In certain embodiments, one or more of the individual components (e.g., heavy and light chains) of the antigen-binding molecules as described herein are derived from chimeric, humanized or fully human antibodies. Methods for making such antibodies are well known in the art. For example, one or more of the heavy and/or light chains of the antigen-binding molecules as described herein can be prepared using VELOCIMMUNE™ technology.

Using VELOCIMMUNE™ technology, for example, or any other similar known method for generating fully human monoclonal antibodies, high affinity chimeric antibodies to CACNG1 are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-CACNG1 antibody. While the constant region selected may vary according to specific use, high affinity antigen-binding and target specificity characteristics reside in the variable region. In certain instances, fully human anti-CACNG1 antibodies are isolated directly from antigen-positive B cells. See, for example, U.S. Pat. No. 6,596,541, Regeneron Pharmaceuticals, VELOCIMMUNE®.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to isolate heavy chain and light chain variable regions to produce fully human bispecific antigen-binding molecules. As such, the fully human bispecific antigen-binding molecules comprise two different heavy chains that associate with the same light chain. (See, e.g., US 2011/0195454). Fully human refers to an antibody, or antigen-binding fragment or immunoglobulin domain thereof, comprising an amino acid sequence encoded by a DNA derived from a human sequence over the entire length of each poly-peptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

Bispecific antigen-binding molecules may be constructed with one heavy chain having a modified Fc domain that abrogates its binding to Protein A, thus enabling a purification method that yields a heterodimeric protein. See, for example, U.S. Pat. No. 8,586,713. As such, the bispecific antigen-binding molecules comprise a first CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU).

### Treatment and Administration

The present disclosure provides anti-CACNG1 antigen-binding proteins (e.g., anti-CACNG1 antibodies or antigen-binding fragments thereof) and anti-CACNG1 protein-drug conjugates which can be used, for example, for delivering a molecular cargo to the body of a subject (e.g., skeletal muscle tissue, in particular, myofibers residing therein), for treating, preventing, or reducing the likelihood of a disease or disorder (e.g., skeletal muscle disease or disorder), in the body of the subject.

In some embodiments, the present disclosure provides methods for administering antigen-binding proteins (e.g., anti-CACNG1 antibodies or antigen-binding fragments thereof) of the present disclosure to a subject, the methods comprising introducing the antigen-binding proteins into the body of the subject. In some embodiments, the present disclosure provides methods for administering the protein-drug conjugates of the present disclosure to a subject, the methods comprising introducing the protein-drug conjugate into the body of the subject.

In some embodiments, the disease or disorder being treated here can be a condition that is not mediated by the activity of CACNG1.

In one aspect, the disclosure provides a pharmaceutical composition comprising an antigen-binding protein (e.g., an anti-CACNG1 antibody or antigen-binding fragment thereof described herein) and/or a protein-drug conjugate described herein together with a pharmaceutically acceptable carrier and/or excipient. The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The pharmaceutical compositions of the disclosure may be in any suitable form (depending upon the desired method of administering to a patient). Suitable compositions and methods of administration are known to those skilled in the art, for example see, Johnson et al., Blood. 2009; 114(3):535-46.

In some embodiments, pharmaceutical compositions may be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The pharmaceutical compositions may comprise the antigen-binding protein or antigen-binding fragment thereof (e.g., the anti-CACNG1 antibody or antigen-binding fragment thereof described herein) and/or the protein-drug conjugates of the disclosure either in the free form or in the form of a pharmaceutically acceptable salt. The term “pharmaceutically acceptable salt” as used herein refers to a derivative of the disclosed protein-drug conjugates wherein the protein-drug conjugates is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids, e.g., acetic acid, benzoic acid, citric acid, propionic acid, glycolic acid, trifluoroacetic acid, pyruvic acid, oxalic acid, malic acid, malonic acid, maleic acid, succinic acid, fumaric acid, tartaric acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a protein-drug conjugates are prepared using a pharmaceutically acceptable base such as sodium hydroxide, potassium hydroxide, ammonium hydroxide, calcium hydroxide, trimethylamine or the like.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

In some embodiments, the protein-drug conjugates described herein may be present in a solution at a concentration of about 1 μg/mL to 50 mg/mL, for example, about 0.1 mg/mL to 10 mg/mL, about 0.2 mg/mL to 5 mg/mL, about 0.5 mg/mL to 8 mg/mL, about 0.8 mg/mL to 12 mg/mL, about 1 mg/mL to 15 mg/mL, about 2 mg/mL to 20 mg/mL, or about 5 mg/mL to 25 mg/mL, or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.25 mg/mL, 1.5 mg/mL, 1.75 mg/mL, 2 mg/mL, 2.25 mg/mL, 2.5 mg/mL, 2.75 mg/mL, 3 mg/mL, 3.25 mg/mL, 3.5 mg/mL, 3.75 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL or 20 mg/mL.

In some embodiments, the antigen-binding proteins or antigen-binding fragments thereof described herein may be present in a solution at a concentration of about 1 μg/mL to 50 mg/mL, for example, about 0.1 mg/mL to 10 mg/mL, about 0.2 mg/mL to 5 mg/mL, about 0.5 mg/mL to 8 mg/mL, about 0.8 mg/mL to 12 mg/mL, about 1 mg/mL to 15 mg/mL, about 2 mg/mL to 20 mg/mL, or about 5 mg/mL to 25 mg/mL, or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.25 mg/mL, 1.5 mg/mL, 1.75 mg/mL, 2 mg/mL, 2.25 mg/mL, 2.5 mg/mL, 2.75 mg/mL, 3 mg/mL, 3.25 mg/mL, 3.5 mg/mL, 3.75 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL or 20 mg/mL.

The pharmaceutical composition may be adapted for administration by any appropriate route such as, e.g., parenteral injections (e.g., via intramuscular, subcutaneous, or intravenous injection).

Such compositions may be prepared by any method known in the art of pharmacy, for example, by mixing the active ingredient with the carrier(s) or excipient(s) under sterile conditions.

In addition, disclosed herein are pharmaceutical dosage forms comprising the antigen-binding proteins and/or the protein-drug conjugates of the disclosure.

Pharmaceutical compositions based on the antigen-binding and/or protein-drug conjugates disclosed herein can be formulated in any conventional manner using one or more physiologically acceptable carriers and/or excipients. The antigen-binding proteins and/or protein-drug conjugates may be formulated for administration by, for example, injection, inhalation, or insulation (either through the mouth or the nose) or by oral, buccal, parenteral administration, or by administration directly to an organ or tissue.

The pharmaceutical compositions can be formulated for a variety of modes of administration, including systemic, topical, or localized administration. Techniques and formulations can be found in, for example, Remington's Pharmaceutical Sciences, Meade Publishing Co., Easton, Pa. In some embodiments, localized injection is used, (e.g., parenteral injection). For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

In some embodiments, the pharmaceutical compositions of the present disclosure may be lyophilized. As a non-limiting example, the obtained lyophilizate can be reconstituted into a hydrous composition by adding a hydrous solvent. In some embodiments, the hydrous composition may be able to be directly administered parenterally (e.g., via intramuscular, subcutaneous, or intravenous injection) to a patient. Therefore, a further embodiment of the present disclosure is a hydrous pharmaceutical composition, obtainable via reconstitution of the lyophilizate with a hydrous solvent.

In some embodiments, the pharmaceutical composition disclosed herein may comprise a lyophilized formulation. As a non-limiting example, the lyophilization formulation may comprise antigen-binding proteins (e.g., antibodies or antigen-binding fragments thereof) and/or protein-drug conjugates of the disclosure, mannitol, and/or TWEEN 80@. As another non-limiting example, the lyophilization formulation may comprise antigen-binding proteins (e.g., antibodies or antigen-binding fragments thereof) and/or protein-drug conjugates disclosed herein, mannitol and poloxamer 188. In some embodiments, the pharmaceutical composition may comprise a lyophilization formulation comprising a reconstituted-liquid composition.

In some embodiments, pharmaceutical compositions of the present disclosure may provide a formulation with an enhanced solubility and/or moistening of the lyophilizate over previously known compositions. As a non-limiting example, enhanced solubility and/or moistening of the lyophilizate may be achieved using an appropriate composition of excipients. In this way, pharmaceutical compositions of the present disclosure comprising antigen-binding proteins (e.g., antibodies or antigen-binding fragments thereof) and/or protein-drug conjugates described herein may be developed to show a desired shelf stability at (e.g., at −20° C., +5° C., or +25° C.) and can be easily resolubilized such that the lyophilizate can be completely dissolved through the use of a buffer or other excipients from seconds up to two or more minutes, with or without the use of an of ultrasonic homogenizer. Furthermore, the composition can be easily provided to a patient in need of treatment via any appropriate delivery route disclosed herein, e.g., parenteral (e.g., via intramuscular, subcutaneous, or intravenous injection), enteral (including oral or rectal), inhalation, or intranasal routes. As a non-limiting example, the pH-value of the resulting solution may be between pH 2.7 and pH 9.

In some embodiments, the pharmaceutical compositions of the present disclosure may be desiccated, e.g., freeze-dried, or a pharmaceutical formulation thereof that includes a pharmaceutically acceptable carrier but substantially lacks water.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g. lactose, microcrystalline cellulose or calcium hydrogen phosphate); lubricants (e.g., magnesium stearate, talc or silica); disintegrants (e.g. potato starch or sodium starch glycolate); or wetting agents (e.g., sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long-acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is then slowly released from the microspheres and absorbed by the surrounding cells present in the selected tissue.

Various delivery systems are known and can be used to administer the pharmaceutical composition as described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition as described herein can be delivered subcutaneously or intravenously with a standard needle and syringe. In addition, with respect to subcutaneous delivery, a pen delivery device readily has applications in delivering a pharmaceutical composition as de-scribed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pens and autoinjector delivery devices have applications in the subcutaneous delivery of a pharmaceutical composition as described herein. Examples include, but are not lim-ited to AUTOPEN™ (Owen Mumford, Inc., Woodstock, UK), DISETRONIC™ pen (Di-setronic Medical Systems, Bergdorf, Switzerland), HUMALOG MIX 75/25™ pen, HU-MALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN348anofiET™, and OPTICLIK™ (sanofi-aventis, Frankfurt, Ger-many), to name only a few. Examples of disposable pen delivery devices having applications in subcutaneous delivery of a pharmaceutical composition as described herein include, but are not limi 349 anofi the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the vector particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

An antigen-binding protein (e.g., an antibody or antigen-binding fragment thereof) and/or a protein-drug conjugate described herein can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutically acceptable carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow-release capsules or microparticles and microspheres and the like can also be employed.

For parenteral administration in an aqueous solution, for example, the solution should be suitably buffered if necessary and the liquid diluent first rendered isotonic with sufficient saline or glucose. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The dose of antigen-binding molecule administered to a patient may vary depending upon the age and the size of the patient, target disease, conditions, route of administration, and the like. The preferred dose is typically calculated according to body weight or body surface area. When an antigen-binding molecule as described herein is used for therapeutic purposes in an adult patient, it may be advantageous to intravenously administer the antigen-binding molecule as described herein normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen-binding molecule may be determined empirically; for example, patient progress can be monitored by peri-odic assessment, and the dose adjusted accordingly. Moreover, interspecies scaling of dosages can be performed using well-known methods in the art (e.g., Mordenti et al., 1991, Pharmaceut. Res. 8:1351).

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Oth-er controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be pre-pared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, poly-ethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

The person responsible for administration will, in any event, determine the appropriate dose for the individual subject. For example, a subject may be administered the antigen-binding proteins (e.g., antibodies or antigen-binding fragments thereof) and/or the protein-drug conjugates described herein on a daily or weekly basis for a time period or on a monthly, bi-yearly or yearly basis.

In addition to the compounds formulated for parenteral administration, (e.g., via intramuscular, subcutaneous, or intravenous injection), other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compounds sucrose as a sweetening agent methyl and propylparabens as preservatives, a dye and flavoring, such as cherry or orange flavor.

The mode of administration of the CACNG1 binding proteins (e.g., the anti-CACNG1 antibodies or antigen-binding fragments thereof described herein) and/or the anti-CACNG1 protein-drug conjugates or composition thereof can vary. Routes of administration include parenteral, non-parenteral, oral, rectal, transmucosal, intestinal, parenteral; intramuscular, subcutaneous, intradermal, intravenous, intraperitoneal, intranasal, intraocular, inhalation, insufflation, topical, cutaneous, intraocular, intravitreal, transdermal or intra-arterial.

Compositions may be administered to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Compositions as disclosed herein can also include adjuvants such as aluminum salts and other mineral adjuvants, tensoactive agents, bacterial derivatives, vehicles and cytokines. Adjuvants can also have antagonizing immunomodulating properties.

Compositions and methods as disclosed herein can also include adjuvant therapy.

The pharmaceutical compositions of the disclosure may be administered directly into the patient, into the affected organ or systemically, or applied ex vivo to cells derived from the patient or a human cell line which are subsequently administered to the patient, or used in vitro to select a subpopulation of cells derived from the patient, which are then re-administered to the patient.

The present disclosure provides a vessel (e.g., a plastic or glass vial, e.g., with a cap or a chromatography column, hollow bore needle or a syringe cylinder) comprising any of the CACNG1 binding proteins or antigen-binding fragments thereof and/or the anti-CACNG1 protein-drug conjugates, or a pharmaceutical formulation comprising a pharmaceutically acceptable carrier thereof.

In some embodiments, CACNG1 binding proteins described herein (e.g., anti-CACNG1 antibodies or antigen-binding fragments thereof described herein) and/or anti-CACNG1 protein-drug conjugates described herein are used for treating, preventing, or reducing the likelihood of a disease or disorder, e.g., a skeletal muscle disease or disorder.

In some embodiments, CACNG1 binding proteins described herein (e.g., anti-CACNG1 antibodies or antigen-binding fragments thereof described herein) and/or anti-CACNG1 protein-drug conjugates described herein may be useful, inter alia, for the treatment, prevention and/or amelioration of any disease or disorder associated with skeletal muscle tissue. For example, CACNG1 binding proteins described herein (e.g., anti-CACNG1 antibodies or antigen-binding fragments thereof described herein) and/or anti-CACNG1 protein-drug conjugates described herein described herein may be useful for the treatment of muscle wasting disorders (e.g., cachexia, glucocorticoid-induced muscle loss, heart failure induced muscle loss, HIV wasting, disuse, aging, etc.) and/or muscular dystrophies/myopathies.

In some embodiments, CACNG1 binding proteins described herein (e.g., anti-CACNG1 antibodies or antigen-binding fragments thereof described herein) and/or anti-CACNG1 protein-drug conjugates described herein are used for treating, preventing, or reducing the likelihood of a skeletal muscle disease or disorder. Non-limiting examples of skeletal muscle diseases or disorders include, but are not limited to, muscular dystrophies (e.g., Duchenne muscular dystrophy (DMD), Becker muscular dystrophy (BMD), congenital muscular dystrophy, distal muscular dystrophy, Emery-Dreifuss muscular dystrophy, facioscapulohumeral muscular dystrophy, Limb-Girdle muscular dystrophy, myotonic muscular dystrophy, and oculopharyngeal muscular dystrophy), muscle atrophies (e.g., spinal muscular atrophies [e.g., Amyotrophic Lateral Sclerosis (ALS), infantile progressive spinal muscular atrophy, intermediate spinal muscular atrophy, juvenile spinal muscular atrophy, adult spinal muscular atrophy] as well as muscle atrophies induced by cancer cachexia, disuse, heart failure, chronic obstructive pulmonary disease, chronic infection, and the like), inflammatory myopathies (e.g., dermatomyositis, polymyositis, inclusion body myositis), diseases of peripheral nerve (e.g., Charcot-Marie tooth disease, Dejerine-Sottas disease, Friedreich's ataxia), diseases of the neuromuscular junction (e.g., Myasthenia gravis, Lambert-Eaton syndrome, botulism), metabolic diseases of the muscle (e.g., acid maltase deficiency, carnitine deficiency, carnitine palmityl transferase deficiency, debrancher enzyme deficiency, lactate dehydrogenase deficiency, mitochondrial myopathy, myoadenylate deaminase deficiency, phosphorylase deficiency, phosphofructokinase deficiency, phosphoglycerate kinase deficiency), central core disease, hyperthyroid myopathy, myotonia congenita, myotubular myopathy, Nemaline myopathy, paramyotonia congenita, periodic paralysis-hypokalemic-hyperkalemic, centronuclear myopathy, Laing distal myopathy, and myofibrillar myopathy. Also provided are methods for treating, preventing, or reducing the likelihood of a skeletal muscle disease or disorder described herein, in a patient in need thereof.

In some embodiments, a muscle disease or disorder (e.g., a skeletal muscle disease or disorder) disclosed herein may include, centronuclear myopathy, Duchenne muscular dystrophy, Facioscapulohumeral muscular dystrophy, familial hypertrophic cardiomyopathy, Friedreich's ataxia, Laing distal myopathy, myofibrillar myopathy, myotonia congenita, myotonic dystrophy, myotubular myopathy, nemaline myopathy, oculopharyngeal muscular dystrophy, Pompe, paramyotonia congenita, and limb girdle muscular dystrophy. In some embodiments, a muscle disease or disorder (e.g., a skeletal muscle disease or disorder) disclosed herein may include any muscle diseases listed in Table 1-3. In some embodiments, defective or differentially regulated (e.g., an upregulated or downregulated) genes corresponding to the muscle diseases disclosed herein that may be targeted with CACNG1 binding proteins described herein (e.g., anti-CACNG1 antibodies or antigen-binding fragments thereof described herein) and/or anti-CACNG1 protein-drug conjugates described herein are also listed in Table 1-3.

Table 1-3. List of muscle diseases and corresponding genes Disease Gene name GenBank Accession No.

Centronuclear myopathy Dynamin 2 (DNM2) NM_001190716; NM_004945; (CNM) NM 001005362;

TABLE 1-3
List of muscle diseases and corresponding genes

| Disease | Gene name | GenBank Accession No. |
|---|---|---|
| Centronuclear myopathy | Dynamin 2 (DNM2) | NM_001190716; NM_004945; |
| (CNM) | | NM_001005362; NM_001005360; |
| | | NM_001005361; NM_007871 |
| | Striated Muscle | NM_001173476; NM_005876 |
| | Enriched Protein | |
| | Kinase (SPEG) | |
| Duchenne muscular | Dystrophin (DMD) | NM_004023; NM_004020; |
| Dystrophy (DMD) | | NM_004018; NM_004012 |
| Facioscapulohumeral | Double Homeobox | NM_001306068; |
| muscular dystrophy | 4 (DUX4) | NM_001363820; |
| (FSHD) | | NM_001205218; |
| | | NM_001293798 |
| | Structural | NM_015295 |
| | Maintenance Of | |
| | Chromosomes | |
| | Flexible Hinge | |
| | Domain Containing | |
| | 1 (SMCHD1) | |
| Familial hypertrophic | Myosin Heavy | NM_000257; NM_080728 |
| cardiomyopathy | Chain 7 (MYH7) | |
| Friedreich's ataxia | Frataxin (FXN) | NM_001161706; NM_181425; |
| (FRDA) | | NM_000144 |
| Inclusion body | Glucosamine (UDP- | NM_001190383; |
| myopathy 2 | N-Acetyl)-2- | NM_001190384; |
| | Epimerase/N- | NM_001128227; NM_005476; |
| | Acetylmannosamine | NM_001190388 |
| | Kinase (GNE) | |
| Laing distal myopathy | Myosin Heavy | NM_000257; NM_080728 |
| | Chain 7 (MYH7) | |
| Myofibrillar myopathy | BAG Cochaperones | NM_004281 |
| | (BAGS) | |
| | Crystallin Alpha B | NM_001885; NM_001330379; |
| | (CRYAB) | NM_001289807; |
| | | NM_001289808 |
| | Desmin (DES) | NM_001927 |
| | DnaJ Heat Shock | NM_005494; NM_058246 |
| | Protein Family | |
| | (Hsp40) Member B6 | |
| | (DNAJB6) | |
| | Four And A Half | NM_001159701; |
| | LIM Domains 1 | NM_001159699; |
| | (FHL1) | NM_001159702; |
| | | NM_001159703; |
| | | NM_001159704; |
| | | NM_001159700; |
| | | NM_001167819; |
| | | NM_001330659; NM_001449; |
| | | NM_001077362 |
| | Filamin C (FLNC) | NM_001458; NM_001127487 |
| | LIM Domain Binding | NM_007078; NM_001171611; |
| | 3 (LDB3) | NM_001171610; |
| | | NM_001080114; |
| | | NM_001080115; |
| | | NM_001080116 |
| | Myotilin (MYOT) | NM_001300911; NM_006790; |
| | | NM_001135940 |
| | Plectin (PLEC) | NM_201378; NM_201379; |
| | | NM_201380; NM_201381; |
| | | NM_201382; NM_201383; |
| | | NM_201384; NM_000445 |
| | Titin (TTN) | NM_133432; NM_133379; |
| | | NM_133437; NM_003319; |
| | | NM_001256850; |
| | | NM_001267550; NM_133378 |
| Myotonia congenita | Chloride Voltage- | NM_000083; NM_013491 |
| (autosomal dominant form, | Gated Channel 1 | |
| Thomsen Disease) | (CLCN1) | |
| Myotonic dystrophy type I | Myotonic dystrophy | NM_001081563; NM_004409; |
| | protein kinase | NM_001081560; |
| | (DMPK) | NM_001081562; |
| | | NM_001288764; |
| | | NM_001288765; |
| | | NM_001288766 |
| Myotonic dystrophy type II | CCHC-Type Zinc | NM_001127192; |
| | Finger Nucleic Acid | NM_001127193; |
| | Binding Protein | NM_001127194; |
| | (CNBP) | NM_001127195; |
| | | NM_001127196; NM_003418 |
| Myotubular myopathy | Myotubularin 1 | NM_000252 |
| | (MTM1) | |
| Oculopharyngeal muscular | Poly(A) Binding | NM_004643 |
| dystrophy | Protein Nuclear 1 | |
| | (PABPN1) | |
| Nemaline myopathy | Nebulin (NEB) | NM_001164507; |
| | | NM_001164508; |
| | | NM_001271208; NM_004543 |
| Paramyotonia congenita | Sodium Voltage- | NM_000334 |
| | Gated Channel | |
| | Alpha Subunit 4 | |
| | (SCN4A) | |
| Pompe | Alpha Glucosidase | NM_000152; NM_001079803; |
| | (GAA) | NM_001079804 |
| | Glycogen Synthase | NM_001161587; NM_002103 |
| | 1 (GYS1) | |

In some embodiments, CACNG1 binding proteins described herein (e.g., anti-CACNG1 antibodies or antigen-binding fragments thereof described herein) and/or anti-CACNG1 protein-drug conjugates described herein can be used to target a muscle disease or disorder that is associated with a gene or gene product such as Double Homeobox 4 (DUX4), myotonic dystrophy protein kinase (DMPK), dystrophin (DMD), F-Box Only Protein 32 (FBX032), Tripartite Motif Containing 63 (TRIM63), Inhibin Subunit Beta A (INHBA), Myostatin (MSTN), Myocyte Enhancer Factor 2D (MEF2D), KLF Transcription Factor 15 (KLF15), Mediator Complex Subunit 1 (MED1), Mediator Complex Subunit 13 (MED13), Protein Phosphatase 1 Regulatory Subunit 3A (PPP1R3A), Myosin Light Chain Kinase (MLCK1), Activin A Receptor Type 1B (ACVR1B), or Type II SH2-domain-containing inositol 5-phosphatase (SHIP2).

In some embodiments, CACNG1 binding proteins described herein (e.g., anti-CACNG1 antibodies or antigen-binding fragments thereof described herein) and/or anti-CACNG1 protein-drug conjugates described herein comprise a CACNG1-binding protein conjugated to interfering nucleic acid (e.g., siRNA) targeting a gene associated with muscle atrophy. The interfering nucleic acid (e.g., siRNA) mediates RNA interference against the gene associated with muscle atrophy, thereby treating, preventing, and/or reducing the likelihood of muscle atrophy in a subject. In some embodiments, the gene associated with muscle atrophy comprises a differentially regulated (e.g., an upregulated or downregulated) gene within the IGF1-Akt-FoxO pathway, the glucocorticoids-GR pathway, the PGC1α-FoxO pathway, the TNFα-NFKB pathway, orthe myostatin-ActRIIb-Smad2/3 pathway. Forexample, the gene associated with muscle atrophy may encodes a ubiquitin ligase (e.g., E3 ubiquitin ligase), Forkhead box transcription factor, a growth factor, deubiquitinating enzyme, or a protein that is involved in glucocorticoid-induced atrophy.

In some embodiments, a gene associated with muscle atrophy described herein encodes a ubiquitin ligase. Exemplary ubiquitin ligases include, but are not limited to, E3 ubiquitin ligases such as Atrogin-1/MAFbx, F-Box protein 30 (Fbxo30), F-Box protein 40 (Fbxo40), muscle RING finger 1 (MuRF1), neural precursor cell expressed developmentally down-regulated protein 4 (Nedd4-1), TNF receptor adaptor protein 6 (TRAF6), or tripartite motif-containing protein 32 (Trim32), and mitochondrial ubiquitin ligases, such as Mitochondrial E3 ubiquitin protein ligase 1 (Mull) and Carboxy terminus of Hsc70 interacting protein (CHIP). In some embodiments, a gene associated with muscle atrophy described herein encodes a Forkhead box transcription factor, such as isoforms Forkhead box protein O1 (FoxO1) and Forkhead box protein 03 (FoxO3). In some embodiments, a gene associated with muscle atrophy described herein encodes a growth factor, such as myostatin. In some embodiments, a gene associated with muscle atrophy described herein encodes a deubiquitinating enzyme, such as Ubiquitin specific peptidase 14 (USP14) and Ubiquitin specific peptidase 19 (USP19). In some embodiments, genes associated with muscle atrophy described herein include those that encode regulated in development and DNA damage response 1 (Redd1), cathepsin L2, TG interacting factor (TGIF1), myogenin, myotonic dystrophy protein kinase (DMPK), histone deacetylase 2 (HDAC2), histone deacetylase 3 (HDAC3), metallothionein 1 L (MT1 L), or metallothionein 1B (MT1 B).

In some embodiments, the muscle atrophy is induced by cancer cachexia, disuse, heart failure, COPD, chronic infection, etc. In some embodiments, the muscle atrophy is a diabetes-associated muscle atrophy. In some embodiments, the muscle atrophy is a cancer cachexia-associated muscle atrophy. In some embodiments, the muscle atrophy is associated with insulin deficiency. In some embodiments, the muscle atrophy is associated with chronic renal failure. In some embodiments, the muscle atrophy is associated with congestive heart failure. In some embodiments, the muscle atrophy is associated with chronic respiratory disease. In some embodiments, the muscle atrophy is associated with a chronic infection. In some embodiments, the muscle atrophy is associated with fasting. In some embodiments, the muscle atrophy is associated with denervation. In some embodiments, the muscle atrophy is associated with sarcopenia, glucocorticoid treatment, stroke, and/or heart attack.

In some embodiments, the muscle disease is myotonic dystrophy type 1 (DM1). In some embodiments, the DM1 is associated with an expansion of CTG repeats in the 3′ UTR of the DMPK gene.

In some embodiments, a subject disclosed herein has a skeletal muscle disease or disorder disclosed herein. In some embodiments, for a subject having a skeletal muscle disease or disorder, a gene or gene product related to that skeletal muscle disease or disorder disclosed herein is knocked down by from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having a skeletal muscle disease or disorder, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having a skeletal muscle disease or disorder, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is about 5%. In some embodiments, the knockdown level is about 10%. In some embodiments, the knockdown level is about 15%. In some embodiments, the knockdown level is about 20%. In some embodiments, the knockdown level is about 25%. In some embodiments, the knockdown level is about 30%. In some embodiments, the knockdown level is about 35%. In some embodiments, the knockdown level is 40%. In some embodiments, the knockdown level is about 45%. In some embodiments, the knockdown level is about 50%. In some embodiments, the knockdown level is about 55%. In some embodiments, the knockdown level is about 60%. In some embodiments, the knockdown level is about 65%. In some embodiments, the knockdown level is 70%. In some embodiments, the knockdown level is about 75%. In some embodiments, the knockdown level is 80%. In some embodiments, the knockdown level is about 85%.

In some embodiments, a subject disclosed herein has a muscular dystrophy. In some embodiments, for a subject having a muscular dystrophy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having a muscular dystrophy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having a muscular dystrophy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, the muscular dystrophy can be, e.g., Duchenne muscular dystrophy (DMD), Becker muscular dystrophy (BMD), congenital muscular dystrophy, distal muscular dystrophy, Emery-Dreifuss muscular dystrophy, facioscapulohumeral muscular dystrophy, Limb-Girdle muscular dystrophy, myotonic muscular dystrophy, or oculopharyngeal muscular dystrophy

In some embodiments, a subject disclosed herein has a muscular atrophy. In some embodiments, for a subject having a muscular atrophy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having a muscular atrophy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having a muscular atrophy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, the muscular atrophy can be, e.g., spinal muscular atrophies, and muscle atrophies induced by cancer cachexia, disuse, heart failure, chronic obstructive pulmonary disease, or chronic infection. Non-limiting examples of spinal muscular atrophies are Amyotrophic Lateral Sclerosis (ALS), infantile progressive spinal muscular atrophy, intermediate spinal muscular atrophy, juvenile spinal muscular atrophy, and adult spinal muscular atrophy.

In some embodiments, a subject disclosed herein has an inflammatory myopathy. In some embodiments, for a subject having an inflammatory myopathy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having an inflammatory myopathy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having an inflammatory myopathy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, the inflammatory myopathy can be, e.g., dermatomyositis, polymyositis, or inclusion body myositis.

In some embodiments, a subject disclosed herein has a disease of a peripheral nerve. In some embodiments, for a subject having a disease of a peripheral nerve, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having a disease of a peripheral nerve, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having a disease of a peripheral nerve, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, the disease of the peripheral nerve can be, e.g., Charcot-Marie tooth disease, Dejerine-Sottas disease or Friedreich's ataxia.

In some embodiments, a subject disclosed herein has a disease of a neuromuscular junction. In some embodiments, for a subject having a disease of a neuromuscular junction, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having a disease of a neuromuscular junction, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having a disease of a neuromuscular junction, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, the disease of the neuromuscular junction can be, e.g., Myasthenia gravis, Lambert-Eaton syndrome or botulism.

In some embodiments, a subject disclosed herein has a metabolic diseases of a muscle. In some embodiments, for a subject having a metabolic diseases of a muscle, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having a metabolic diseases of a muscle, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having a metabolic diseases of a muscle, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, the metabolic disease of the muscle can be, e.g., acid maltase deficiency, carnitine deficiency, carnitine palmityl transferase deficiency, debrancher enzyme deficiency, lactate dehydrogenase deficiency, mitochondrial myopathy, myoadenylate deaminase deficiency, phosphorylase deficiency, phosphofructokinase deficiency or phosphoglycerate kinase deficiency.

In some embodiments, a subject disclosed herein has central core disease. In some embodiments, for a subject having central core disease, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having central core disease, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having central core disease, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has hyperthyroid myopathy. In some embodiments, for a subject having hyperthyroid myopathy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having hyperthyroid myopathy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having hyperthyroid myopathy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has myotonia congenita. In some embodiments, for a subject having myotonia congenita, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having myotonia congenita, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having myotonia congenita, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has myotubular myopathy. In some embodiments, for a subject having myotubular myopathy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having myotubular myopathy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having myotubular myopathy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has Nemaline myopathy. In some embodiments, for a subject having Nemaline myopathy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having Nemaline myopathy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having Nemaline myopathy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has paramyotonia congenita. In some embodiments, for a subject having paramyotonia congenita, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having paramyotonia congenita, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having paramyotonia congenita, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has periodic paralysis-hypokalemic-hyperkalemic. In some embodiments, for a subject having periodic paralysis-hypokalemic-hyperkalemic, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having periodic paralysis-hypokalemic-hyperkalemic, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having periodic paralysis-hypokalemic-hyperkalemic, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has centronuclear myopathy. In some embodiments, for a subject having centronuclear myopathy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having centronuclear myopathy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having centronuclear myopathy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has Laing distal myopathy. In some embodiments, for a subject having Laing distal myopathy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having Laing distal myopathy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having Laing distal myopathy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has myofibrillar myopathy. In some embodiments, for a subject having myofibrillar myopathy, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having myofibrillar myopathy, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having myofibrillar myopathy, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

In some embodiments, a subject disclosed herein has a disease or disorder disclosed in Tables 1-3. In some embodiments, for a subject having a disease or disorder disclosed in Tables 1-3, a gene or gene product disclosed herein is knocked down by from at least about 5% to at least about 10%, from at least about 5% to at least about 15%, from at least about 5% to at least about 20%, from at least about 5% to at least about 25%, from at least about 5% to at least about 30%, from at least about 5% to at least about 35%, from at least about 5% to at least about 40%, from at least about 5% to at least about 45%, from at least about 5% to at least about 50%, from at least about 10% to at least about 15%, from at least about 10% to at least about 20%, from at least about 10% to at least about 25%, from at least about 10% to at least about 30%, from at least about 10% to at least about 35%, from at least about 10% to at least about 40%, from at least about 10% to at least about 45%, from at least about 10% to at least about 50%, from at least about 15% to at least about 20%, from at least about 15% to at least about 25%, from at least about 15% to at least about 30%, from at least about 15% to at least about 35%, from at least about 15% to at least about 40%, from at least about 15% to at least about 45%, from at least about 15% to at least about 50%, from at least about 20% to at least about 25%, from at least about 20% to at least about 30%, from at least about 20% to at least about 35%, from at least about 20% to at least about 40%, from at least about 20% to at least about 45%, from at least about 20% to at least about 50%, from at least about 25% to at least about 30%, from at least about 25% to at least about 35%, from at least about 25% to at least about 40%, from at least about 25% to at least about 45%, from at least about 25% to at least about 50%, from at least about 30% to at least about 35%, from at least about 30% to at least about 40%, from at least about 30% to at least about 45%, from at least about 30% to at least about 50%, from at least about 35% to at least about 40%, from at least about 35% to at least about 45%, from at least about 35% to at least about 50%, from at least about 40% to at least about 45%, from at least about 40% to at least about 50%, from at least about 45% to at least about 50%, or more upon administration of a protein-drug conjugate (e.g., an antibody-drug conjugate) disclosed herein as compared to a subject who is not administered the protein-drug conjugate. In some embodiments, for a subject having a disease or disorder disclosed in Tables 1-3, a gene or gene product disclosed herein is knocked down by from at least about 50% to at least about 60%, from at least about 50% to at least about 70%, from at least about 50% to at least about 80%, from at least about 50% to at least about 90%, more than 60%, from at least about 60% to at least about 70%, from at least about 60% to at least about 80%, from at least about 60% to at least about 90%, more than at least about 70%, from at least about 70% to at least about 80%, from at least about 70% to at least about 90%, more than at least about 80%, from at least about 80% to at least about 90%, more than 90%, from at least about 90% to at least about 95%, from at least about 90% to at least about 98%, more than 95%, from at least about 95% to at least about 98%, more than at least about 98%, or more than at least about 99% upon administration of a protein-drug conjugate (e.g., an antibody-drug conjugate) disclosed herein as compared to a subject who is not administered the protein-drug conjugate. In some embodiments, for a subject having a disease or disorder disclosed in Tables 1-3, a gene or gene product disclosed herein is knocked down by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or even 100% upon administration of a protein-drug conjugate (e.g., an antibody-drug conjugate) disclosed herein as compared to a subject who is not administered the protein-drug conjugate. In some embodiments, the knockdown level is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100%. In some embodiments, the knockdown level is 5%. In some embodiments, the knockdown level is 10%. In some embodiments, the knockdown level is 15%. In some embodiments, the knockdown level is 20%. In some embodiments, the knockdown level is 25%. In some embodiments, the knockdown level is 30%. In some embodiments, the knockdown level is 35%. In some embodiments, the knockdown level is 40%. In some embodiments, the knockdown level is 45%. In some embodiments, the knockdown level is 50%. In some embodiments, the knockdown level is 55%. In some embodiments, the knockdown level is 60%. In some embodiments, the knockdown level is 65%. In some embodiments, the knockdown level is 70%. In some embodiments, the knockdown level is 75%. In some embodiments, the knockdown level is 80%. In some embodiments, the knockdown level is 85%.

As used herein, the term “subject” refers to a mammal (e.g., rat, mouse, cat, dog, cow, sheep, horse, goat, rabbit), preferably a human, for example, in need of prevention and/or treatment of a disease or disorder described herein.

The present disclosure includes therapeutically-effective combinations including a CACNG1 binding protein described herein (e.g., an anti-CACNG1 antibody or antigen-binding fragment thereof described herein) and/or an anti-CACNG1 protein-drug conjugate described herein, in association with one or more further therapeutic agents. A further therapeutic agent that is administered to a subject in association with a CACNG1 binding protein described herein (e.g., an anti-CACNG1 antibody or antigen-binding fragment thereof described herein) and/or an anti-CACNG1 protein-drug conjugate is administered to the subject in accordance with the Physicians' Desk Reference 2003 (Thomson Healthcare, 57th edition (Nov. 1, 2002)). The CACNG1 binding protein and/or the anti-CACNG1 protein-drug conjugate and the further therapeutic agent can be in a single composition or in separate compositions.

Methods for treating, preventing, or reducing the likelihood of a disease or disorder in a subject in need of said treatment, prevention, or reduction of likelihood of the disease or disorder by administering a CACNG1 binding protein described herein (e.g., an anti-CACNG1 antibody or antigen-binding fragment thereof described herein) and/or an anti-CACNG1 protein-drug conjugate described herein, in association with a further therapeutic agent, are part of the present disclosure. Compositions comprising the CACNG1 binding protein and/or the anti-CACNG1 protein-drug conjugate in association with one or more further therapeutic agents also form part of the present disclosure.

The term “in association with” indicates that components, a CACNG1-binding protein, e.g., antibody or antigen-binding fragment thereof of the present disclosure, along with another agent such as methotrexate, can be formulated into a single composition, e.g., for simultaneous delivery, or formulated separately into two or more compositions (e.g., a kit including each component). Components administered in association with each another can be administered to a subject at a different time than when the other component is administered; for example, each administration may be given non-simultaneously (e.g., separately or sequentially) at intervals over a given period of time. Separate components administered in association with each another may also be administered sequentially, though essentially simultaneously, during the same administration session. Moreover, the separate components administered in association with each another may be administered to a subject by the same or by a different route.

In some embodiments, a CACNG1 binding protein (e.g., an anti-CACNG1 antibody or antigen-binding fragment thereof described herein) and/or an anti-CACNG1 protein-drug conjugate may be administered in association with a further therapeutic agent such as, for example, Astaxanthin (e.g., HI-Q 300, Carnirich-M, Ovasafe, Prorac, Wasolvit Gold, EN-Q 300, Eyetamin Gold, Fortify, Lenova-M, Rqual-Gold), Drotaverine (e.g., Dove, Drovera, Drot, Samspas, Doverin, Drovet, Spacovin, Din, Spasmoter, Drotavin), Eperisone (e.g., Rapisone, Epry, Enzoril, Skelact, Myosone, Rapisone SR), Fenoverine, (e.g., Spasmopriv, Spasmopriv, Methocarbamol (e.g., Robinax, Robiflam, Robiflam, Robilid, Methoriv-N, Flexinol, Mylax, Neuromol-MR, Robinaxol, Methoriv-P), Nusinersen, Pipcuronium (Arduan), or Valbenazine for the treatment of muscular dystrophy.

An effective or therapeutically-effective dose of a CACNG1 binding protein (e.g., a CACNG1 antibody or antigen-binding fragment thereof described herein) and/or an CACNG1 protein-drug conjugate, for treating, preventing, or reducing the likelihood of an CACNG1-mediated condition refers to the amount of the antigen-binding protein sufficient to alleviate one or more signs and/or symptoms of the disease or condition in the treated subject, whether by inducing the regression or elimination of such signs and/or symptoms or by inhibiting the progression of such signs and/or symptoms. In an embodiment of the disclosure, an effective or therapeutically-effective dose of CACNG1-binding protein is about 2-30 mg/kg about once a month. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses are separated by days or weeks or months.

A symptom is a manifestation of disease apparent to the patient himself, while a sign is a manifestation of disease that the physician perceives. Reduction, fully or in part, of a sign or symptom may be referred to as alleviation of the sign or symptom.

In an embodiment, an effective or therapeutically-effective dose of CACNG1 binding protein (e.g., a CACNG1 antibody or antigen-binding fragment thereof described herein) and/or anti-CACNG1 protein-drug conjugate is about 1 mg/kg and 50 mg/kg body weight. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses are separated by days or weeks.

In some embodiments, CACNG1 binding proteins (e.g., a CACNG1 antibodies or antigen-binding fragments thereof described herein) and/or anti-CACNG1 protein-drug conjugates as described herein, or pharmaceutical compositions thereof, may be administered in accordance with a stepwise dosing regimen. Stepwise dosing of a composition can refer to breaking up (i.e., dividing) dosing of the same composition over multiple administrations. In some embodiments, the dosing of the same composition is broken up once, twice, three times, four times, five times, six times, seven times, eight times, nine times, ten times, or more, over the time course of the treatment of a subject which can occur over any number of days, weeks, or years. In some embodiments, when a stepwise dose regimen is used in the administration of CACNG1 binding proteins (e.g., a CACNG1 antibodies or antigen-binding fragments thereof described herein) and/or anti-CACNG1 protein-drug conjugates, the stepwise dosing regimen may result in a gradual increase in therapeutic transgene levels with each administration of the anti-CACNG1 protein-drug conjugate.

In some embodiments, CACNG1 binding proteins (e.g., a CACNG1 antibodies or antigen-binding fragments thereof described herein) and/or anti-CACNG1 protein-drug conjugates as described herein may be used in diagnostic assays for CACNG1, e.g., detecting its expression in specific cells, tissues, etc., e.g., as a reagent to identify/label skeletal muscle fibers. Various diagnostic and prognostic assay techniques known in the art may be used, such as competitive binding assays, direct or indirect sandwich assays and immunoprecipitation assays conducted in either heterogeneous or homogeneous phases (Zola (1987) Monoclonal Antibodies: A Manual of Techniques, CRC Press, Inc. pp. 147-1581). The antibodies used in the assays can be labeled with a detectable moiety. The detectable moiety should be capable of producing, either directly or indirectly, a detectable signal. Any method known in the art for conjugating the antibody to the detectable moiety may be employed.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1. Generation of Anti-Human CACNG1 Antibodies

Anti-human CACNG1 antibodies were obtained by immunizing a mouse (e.g., an engineered mouse comprising DNA encoding human immunoglobulin heavy and human kappa light chain variable regions), with human CACNG1.

Following immunization, splenocytes were harvested from each mouse and either (1) fused with mouse myeloma cells to preserve their viability and form hybridoma cells and screened for human CACNG1 specificity, or (2) B-cell sorted (as described in US 2007/0280945A1) using a either a human CACNG1 fragment as the sorting reagent that binds and identifies reactive antibodies (antigen-positive B cells).

Chimeric antibodies to human CACNG1 were initially isolated having a human variable region and a mouse constant region using, e.g., VELOCIMMUNE technology as described in U.S. Pat. Nos. 7,105,348; 8,642,835; and 9,622,459, each of which is incorporated herein by reference.

In some antibodies, for testing purposes, mouse constant regions were replaced with a desired human constant region, for example wild-type human CH or modified human CH (e.g. IgG1, IgG2 or IgG4 isotypes), and light chain constant region (CL), to generate a fully human anti-hCACNG1 antibody, or antigen binding portion thereof. While the constant region selected may vary according to specific use, high affinity antigen-binding and target specificity characteristics reside in the variable region.

Certain biological properties of the exemplary anti-human CACNG1 antibodies generated in accordance with the methods of this Example are described in detail in the Examples set forth below.

### Example 2. Biacore Binding Kinetics of Anti-CACNG1 Antibodies in an Antigen Capture Format to Human CACNG1 Nanodisc

This Example relates to evaluation of binding kinetics of purified anti-CACNG1 antibodies described herein to a human CACNG1 nanodisc capture surface using real-time surface plasmon resonance biosensor technology with a Biacore T200 instrument. Table 2-1 shows a description of the anti-CACNG1 antibodies tested in the present Example.

TABLE 2-1
Anti-CACNG1 antibodies tested

| REGN #/Ab PID # | Lot # | Common Name |
|---|---|---|
| H2aM31929N/REGN10728 | H2aM31929N-L1 | anti-CACNG1 mAb |
| H2aM31944N | H2aM31944N-L1 | anti-CACNG1 mAb |
| H4H31265P2/REGN5972 | H4H31265P2-L1 | anti-CACNG1 mAb |
| H2aM31941N | H1M31941N-L1 | anti-CACNG1 mAb |
| REGN7660 | REGN7660-L1 | anti-CACNG1 mAb |
| REGN9909 | REGN9909-L1 | anti-CACNG1 mAb |
| REGN10713 | REGN10713-L1 | anti-CACNG1 mAb |
| REGN10715 | REGN10715-L1 | anti-CACNG1 mAb |
| REGN10716 | REGN10716-L1 | anti-CACNG1 mAb |
| REGN10717 | REGN10717-L1 | anti-CACNG1 mAb |
| REGN10783 | REGN10783-L1 | anti-CACNG1 mAb |

Equilibrium dissociation constants (K_{D} values) for nanodisc embedded human CACNG1 expressed with a C-terminal PΔΔCt RE-Flag-His tag (human CACNG1 nanodisc; concentration 0.82 mg/ml) binding to purified anti-CACNG1 antibodies were determined using a real-time surface plasmon resonance biosensor technology with a Biacore T200 instrument. The CM5 Biacore sensor surface was derivatized by amine coupling with a monoclonal mouse anti-His antibody (Cytiva; Marlborough, MA). All Biacore binding studies were performed in a buffer composed of 0.01M HEPES, 0.15M NaCl, 1 mM CaCl₂, 0.5 mM MgCl₂, pH 7.4 (HBS-N++ running buffer). Different concentrations of anti-CACNG1 antibodies (ranging from 300 nM to 12 nM in 5-fold serial dilutions) prepared in HBS-N++ running buffer were injected over the captured human CACNG1 nanodisc at a flow rate of 30 μL/minute. Antibody association was monitored for 2 minutes while dissociation was monitored in HBS-N++ running buffer for 5 minutes. At the end of each cycle, the human CACNG1 nanodisc capture surface was regenerated using three 10 second injections of 10 mM Gly pH1.5. All binding kinetics experiments were performed at 25° C.

The specific SPR-Biacore sensorgrams were obtained by a double referencing procedure. The double referencing was performed by first subtracting the signal of each injection over a reference surface (anti-His) from the signal over the experimental surface (anti-His captured human CACNG1 nanodisc) thereby removing contributions from refractive index changes. In addition, running buffer injections were performed to allow subtraction of the signal changes resulting from the dissociation of captured antibodies from the coupled anti-His surface. Kinetic association (kₐ) and dissociation (kₐ) rate constants were determined by fitting the real-time sensorgrams to a 1:1 binding model using Scrubber v2.0c curve fitting software. Binding dissociation equilibrium constants (K_{D}) and dissociative half-lives (t %) were calculated from the kinetic rate constants as:

$${K}_{D} ( M ) = \frac{kd}{ka} , and t 1 / 2 ( min ) = \frac{ln ( 2 )}{60 * kd}$$

Anti-CACNG antibodies kinetic results are presented in Table 2-2. As shown in Table 2-2, all of the antibodies of the disclosure bound to surface-captured human CACNG1 nanodisc with several antibodies of the disclosure binding with single digit nM or triple digit pM affinities.

TABLE 2-2
Kinetic and Equilibrium Binding Parameters of anti-CACNG1 antibodies
to surface-captured human CACNG1 nanodisc at 25° C.

| | CACNG1 | mAb | | | | |
|---|---|---|---|---|---|---|
| | nanodisc | bound at | | | | |
| | captured | 300 nM | kₐ | k_{d} | K_{D} | t½ |
| REGN #/Ab PID # | (RU) | (RU) | (1/Ms) | (1/s) | (M) | (min) |
| H2aM31929N/REGN10728 | 122 ± 0.7 | 25 | 7.50E+05 | 5.59E−04 | 7.45E−10 | 20.7 |
| H2aM31944N | 112 ± 0.6 | 16 | 2.31E+05 | 3.95E−03 | 1.71E−08 | 2.9 |
| H4H31265P2/REGN5972 | 140 ± 1.3 | 14 | 1.04E+05 | 8.40E−03 | 8.10E−08 | 1.4 |
| H2aM31941N | 126 ± 0.9 | 19 | 3.42E+05 | 4.06E−04 | 1.19E−09 | 28.4 |
| REGN7660 | 209 ± 1.9 | 23 | 1.93E+06 | 2.43E−02 | 1.26E−08 | 0.5 |
| REGN10712 | 171 ± 1.7 | 31 | 7.33E+05 | 3.32E−03 | 4.53E−09 | 3.5 |
| REGN9908 | 194 ± 0.6 | 21 | 4.19E+05 | 4.02E−03 | 9.57E−09 | 2.9 |
| REGN9909 | 191 ± 3.3 | 22 | 3.31E+05 | 1.37E−03 | 4.15E−09 | 8.4 |
| REGN10713 | 167 ± 1 | 53 | 7.42E+04 | 4.72E−05 | 6.35E−10 | 245.0 |
| REGN10715 | 159 ± 1.5 | 31 | 1.52E+05 | 3.01E−04 | 1.98E−09 | 38.4 |
| REGN10716 | 152 ± 1.9 | 31 | 7.30E+05 | 8.36E−05 | 1.14E−10 | 138.2 |
| REGN10717 | 148 ± 1.9 | 7 | 2.94E+05 | 3.04E−02 | 1.04E−07 | 0.4 |
| REGN10783 | 139 ± 0.6 | 9 | 1.36E+05 | 1.27E−02 | 9.35E−08 | 0.9 |

### Example 3. Effects of CACNG1 Antibodies on Myotube Calcium Flux

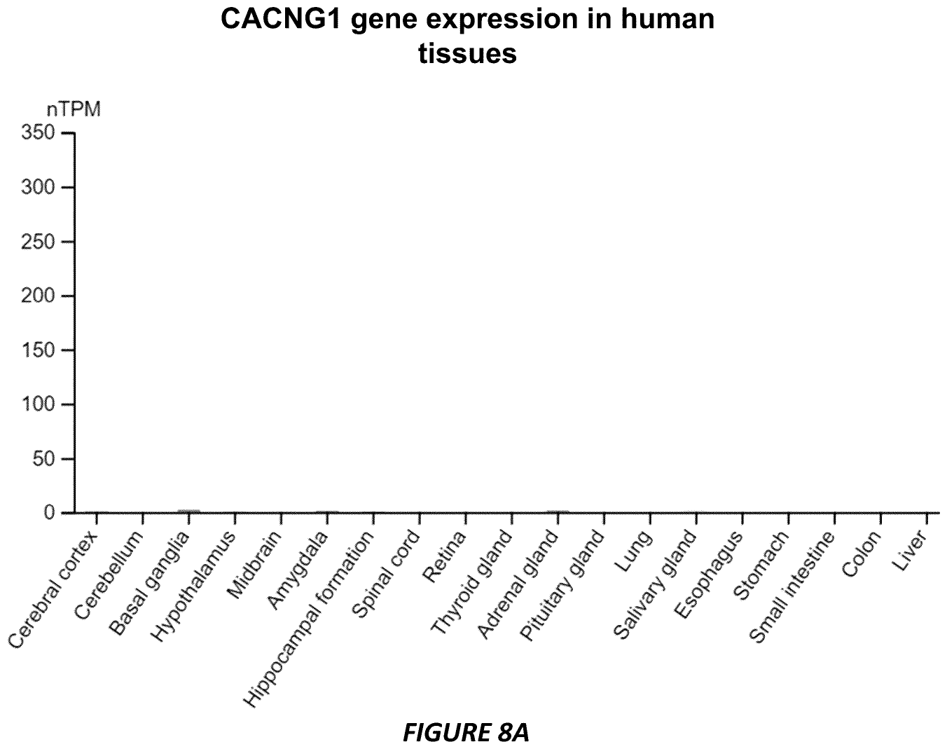

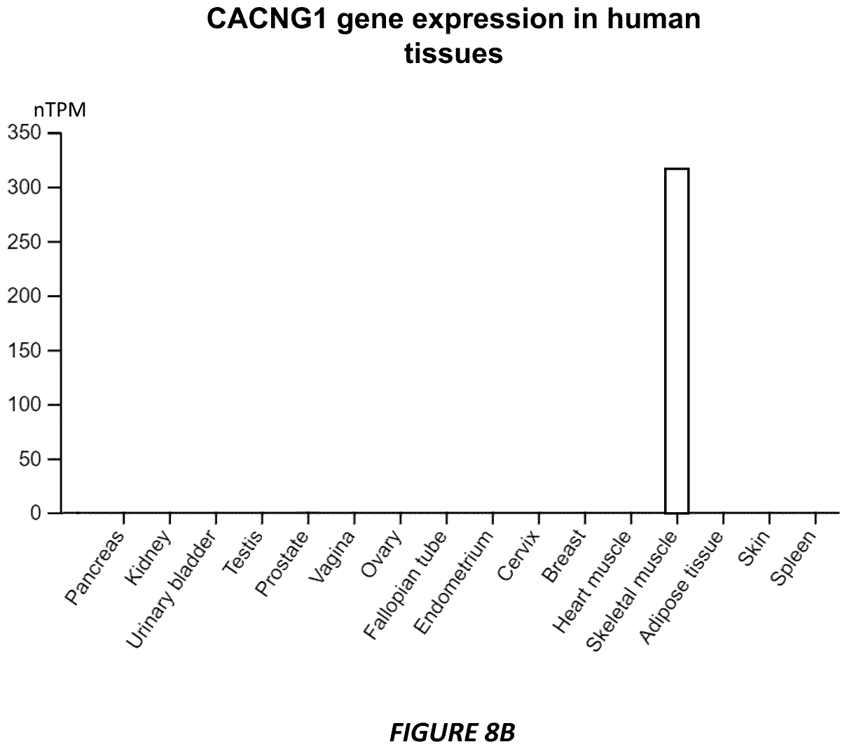

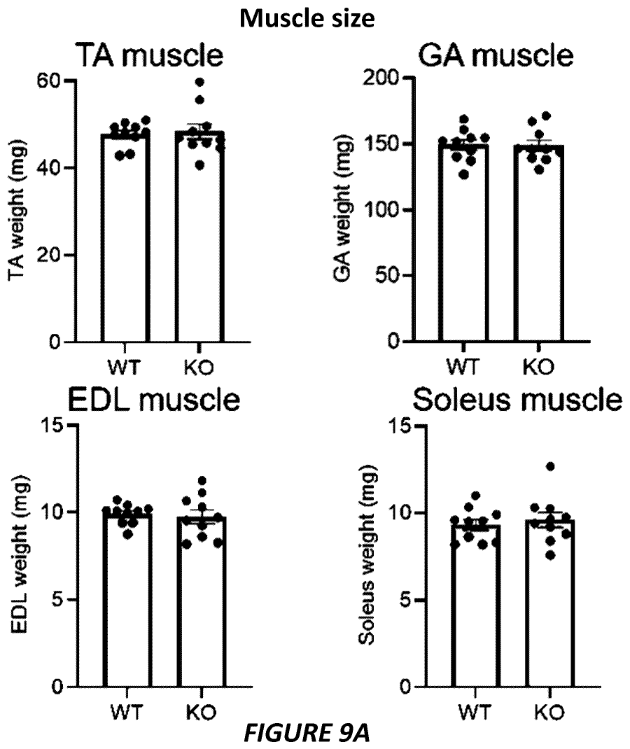

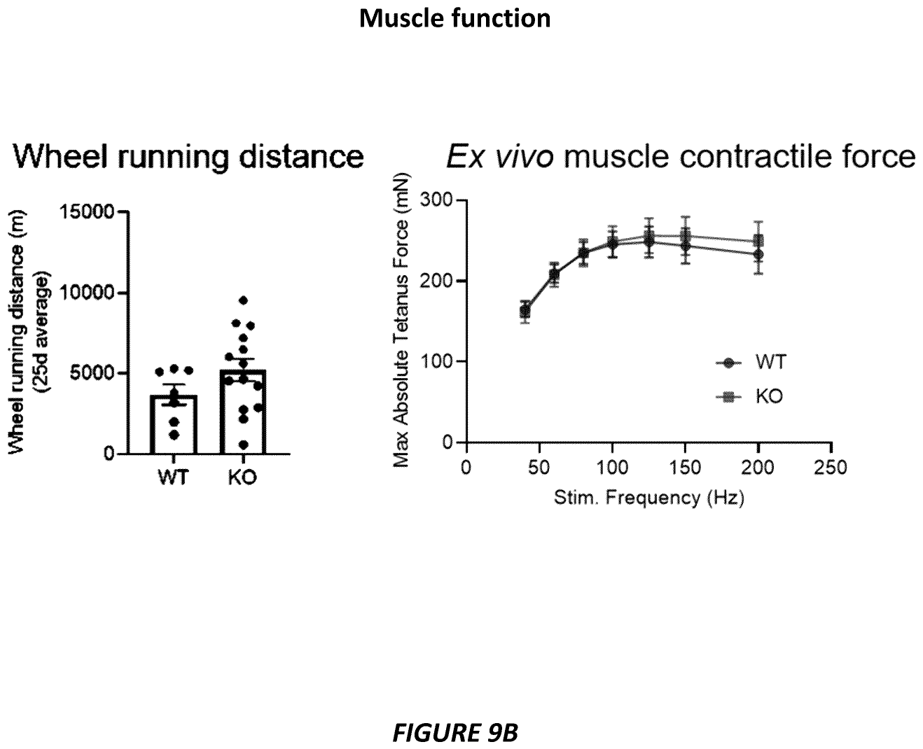

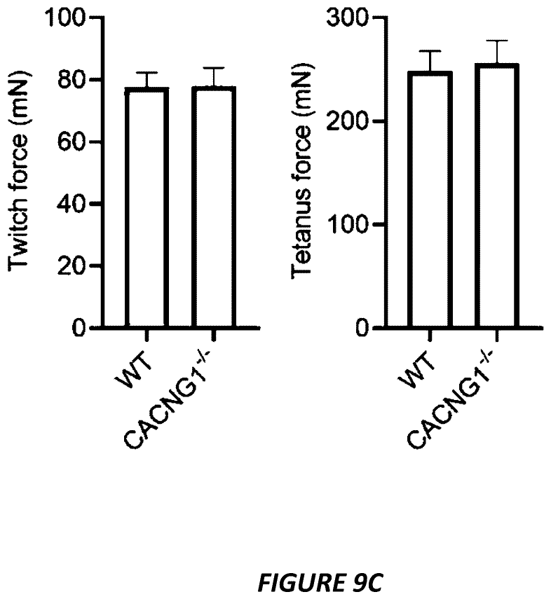

CACNG1 is the γ subunit of the skeletal muscle-specific L-type calcium channel, (dihydropyridine receptor) and is highly and specifically expressed in human skeletal muscle tissue, (GTEx Portal) (FIGS. 8A-8B). To determine whether genetic deletion of CACNG1 disrupts normal muscle function, CACNG1 knockout mice were generated. 224 bp directly following the mouse Cacng1 start ATG was replaced with a lacZ reporter cassette such that the lacZ coding sequence was in frame with the Cacng1 ATG. Following the lacZ coding sequence was an SV40 poly(A) signal, additional cloning sequence, and a loxP recombination site where an antibiotic resistance cassette was removed by self-deleting technology. The 3′ 5 bp of Cacng1 exon 1 was retained, following the IoxP site. In this knockout mouse, genetic deletion of CACNG1 appears to have no major impact on skeletal muscle weights (FIG. 9A). Additionally, genetic deletion of CACNG1 had no impact on mouse voluntary running capacity, assessed by wheel running distance (Sable Systems) (FIG. 9B) or ex vivo muscle force production, measured by stimulating EDL muscles at various frequencies, as well as measuring maximal twitch and tetanic force (Aurora Scientific, Inc.) (FIGS. 9B-9C).

In addition to demonstrating that CACNG1 is not required for normal muscle function, the present Example was designed to determine whether antibodies against CACNG1 also do not impact muscle function. A calcium flux assay was performed on human myotubes that were incubated with CACNG1 antibodies to determine whether these antibodies affect acetylcholine-induced calcium release. Table 3-1 shows a description of the anti-CACNG1 antibodies tested in the present Example.

TABLE 3-1
Anti-CACNG1 antibodies tested

| REGN # | Description |
|---|---|
| REGN5972 | CACNG1 hIgG1 antibody |
| REGN10728 | CACNG1 hIgG4 uber stealth antibody |
| H2aM31944N | CACNG1 mIgG2a antibody |
| REGN3892 | Isotype control hIgG1 antibody |
| REGN1945 | Isotype control hIgG4 antibody |
| REGN1097 | Isotype control mIgG2a antibody |

Human skeletal myoblasts (Cook Myosite, Inc.) were plated at 10,000 cells/well of a 96-well plate and were differentiated for 7 days into myotubes. On the final day of differentiation, media was replaced with 50 μL of FLIPR calcium 5 dye with probenecid (Invitrogen) and 50 μL of assay buffer (0.1% BSA-DMEM) per well. CACNG1 and isotype control antibodies were serial diluted in assay buffer and added to the cells and incubated at 37° C. in a 5% CO₂ incubator for 1 hour prior to the calcium flux assay. Nicardipine hydrochloride (Sigma) was added to untreated wells to serve as a control for calcium channel blockade. After 1 hour, acetylcholine (Sigma) was added at a 20 μM final concentration to induce myotube calcium release, and plates were assayed on a FLIPR Tetra (Molecular Devices, LLC).

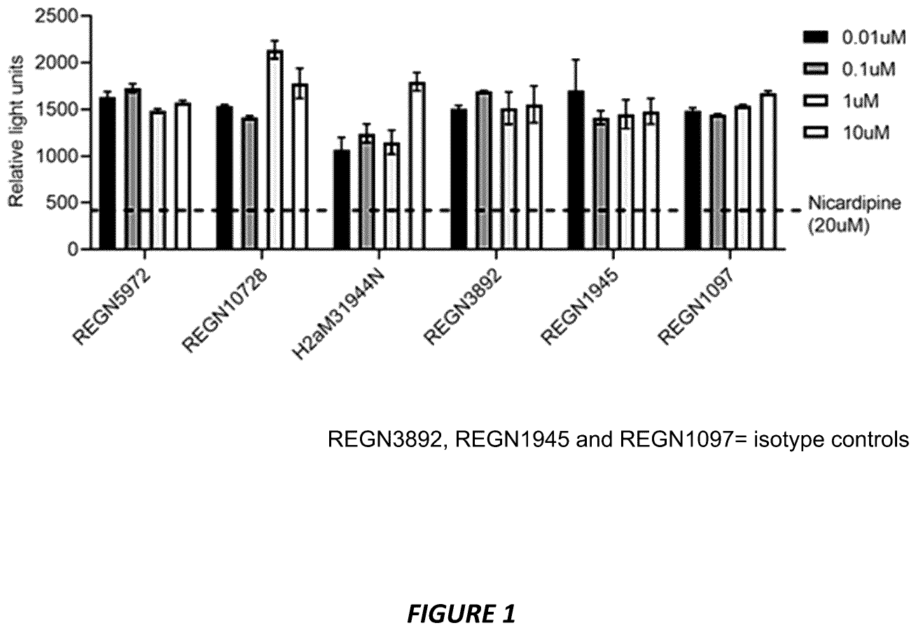

Nicardipine substantially reduced human myotube calcium release, while none of the CACNG1 antibodies or isotype control antibodies had any major impact on calcium flux (FIG. 1). Overall, this demonstrates that CACNG1 antibodies of the present disclosure do not block calcium release in human myotubes cultured in vitro.

### Example 4. CACNG1 Antibody Binding and Internalization in Myofibers Ex Vivo

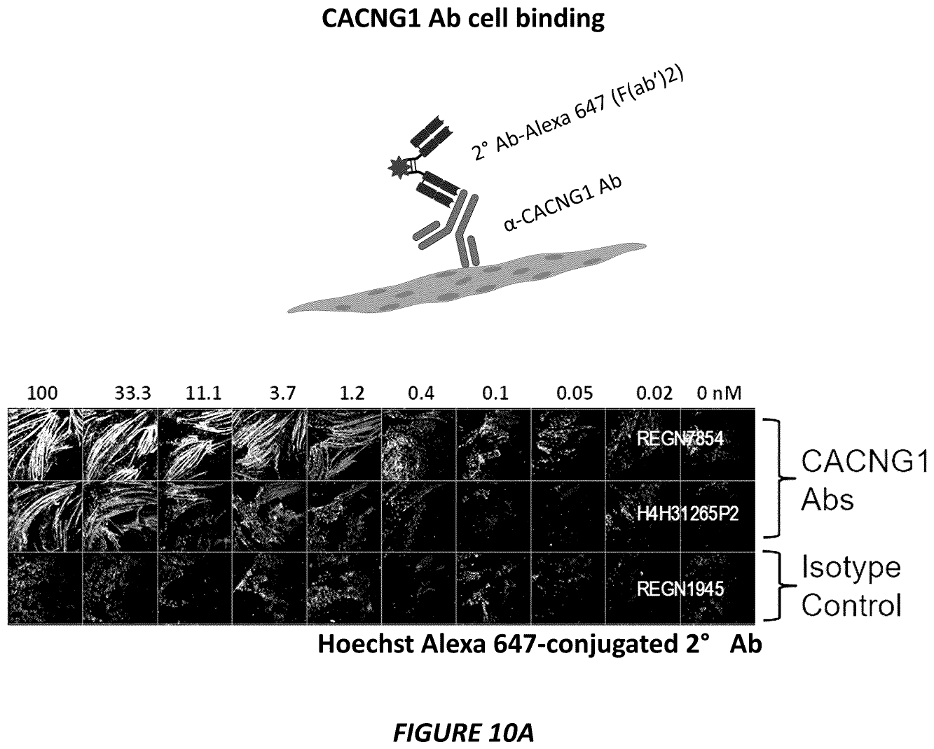

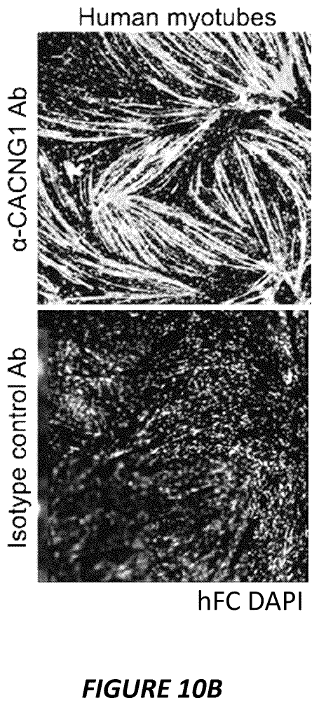

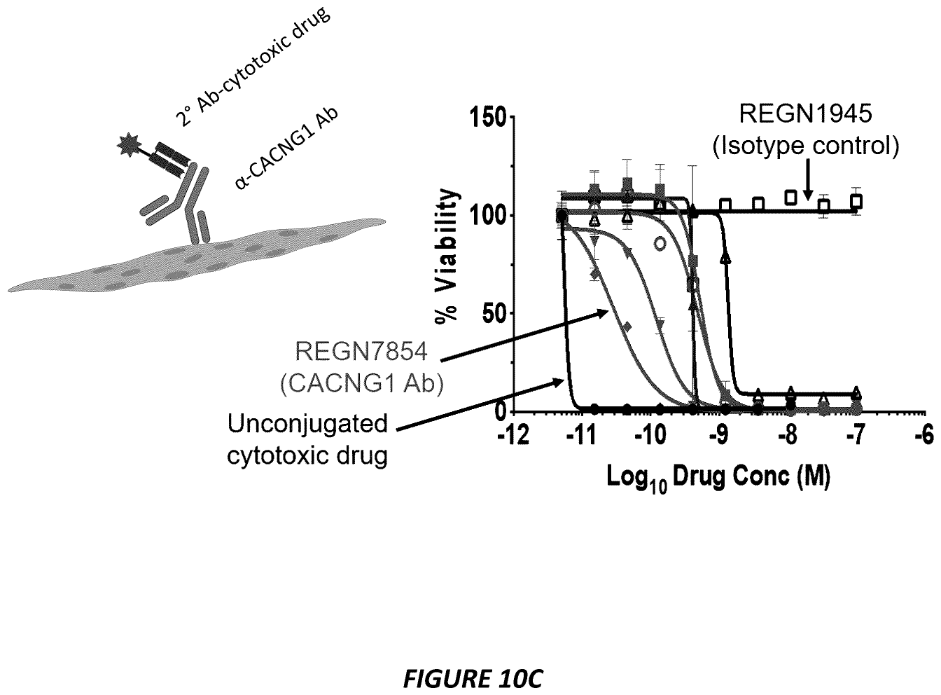

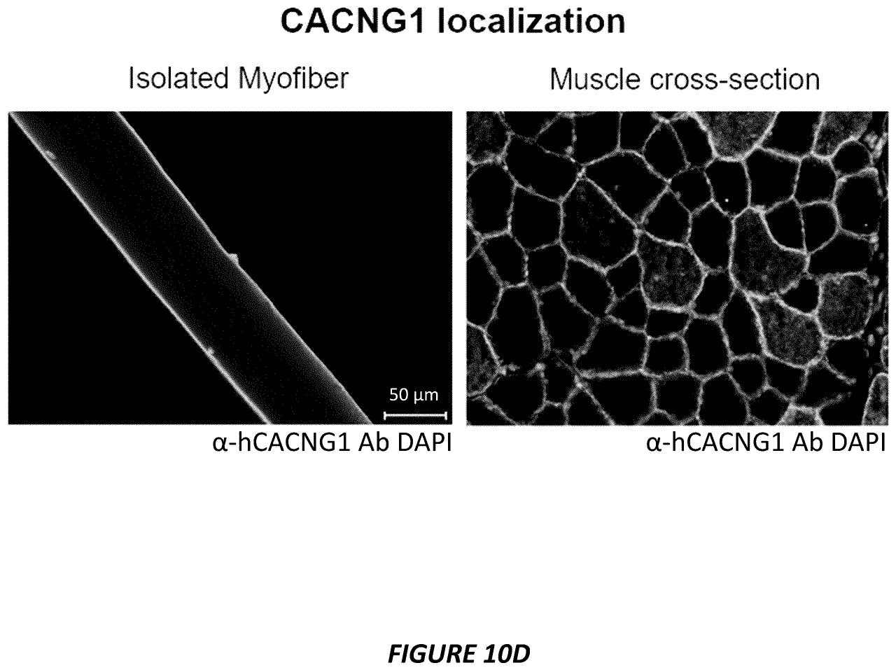

CACNG1 antibodies were confirmed to bind to live human myotubes by incubating with various concentrations of antibody for 30 minutes, washing, incubating with APC-conjugated secondary antibody (Thermo Fisher) for 30 minutes, washing, fixing, counterstaining for Hoescht, and imaging on a fluorescent microscope (Zeiss) (see, e.g., FIG. 10A-B). Following confirmation of CACNG1 antibody binding to human myotubes, a subset of antibodies was tested for binding to fully mature myofibers ex vivo. Table 4-1 shows a description of the anti-CACNG1 antibodies tested in the present Example.

TABLE 4-1
Anti-CACNG1 antibodies tested

| REGN # | Description |
|---|---|
| H1M31941N | CACNG1 mIgG1 antibody |
| REGN5972 | CACNG1 hIgG1 antibody |
| REGN10728 | CACNG1 hIgG4 uber stealth antibody |
| REGN653 | Isotype control mIgG1 antibody |
| REGN1945 | Isotype control hIgG4 antibody |
| REGN4439 | Isotype control hIgG4 uber stealth antibody |

Single myofibers were isolated from either wildtype mice, mice that were homozygous for the deletion of CACNG1 (referred to as CACNG1 knockout mice), or mice that were homozygous for the expression of human CACNG1 in place of mouse CACNG1 (referred to as CACNG1^{Hu/Hu}). The gastrocnemius muscle was removed, collagenase digested, and single myofibers were isolated, washed, and incubated overnight at 37° C. at 5% CO₂, in DMEM+10% horse serum. Following overnight incubation, single myofibers were incubated with 100 nM of each CACNG1 antibody or an isotype control antibody for 30 minutes. Myofibers were then washed twice in DMEM+10% horse serum, and subsequently incubated with 10 μg/mL of fluorescent-conjugated secondary antibodies for 30 minutes, washed twice in DMEM+10% horse serum, and then fixed with 4% paraformaldehyde (PFA) for 15 minutes at room temperature. Single fibers were then washed twice with PBS, stained with Hoechst for 5 minutes, washed once more with PBS, then transferred to microscope slides, cover slipped, and imaged using a Zeiss LSM 710 confocal microscope.

Separately, to determine whether a CACNG1 antibody of the present disclosure can internalize in myofibers ex vivo, Alexa 647 (A647) fluorophore was directly conjugated to the CACNG1 antibody and an isotype control antibody. Single myofibers were isolated, washed, and incubated overnight. The following day, myofibers were incubated with 100 nM of the A647-conjugated antibodies for 30 minutes, 4 hours, or 8 hours, and were then washed twice with PBS. Myofibers were then fixed with 4% PFA for 15 minutes, stained with Hoechst for 5 minutes, washed once more with PBS, then transferred to microscope slides, cover slipped, and imaged using a Zeiss LSM 710 confocal microscope.

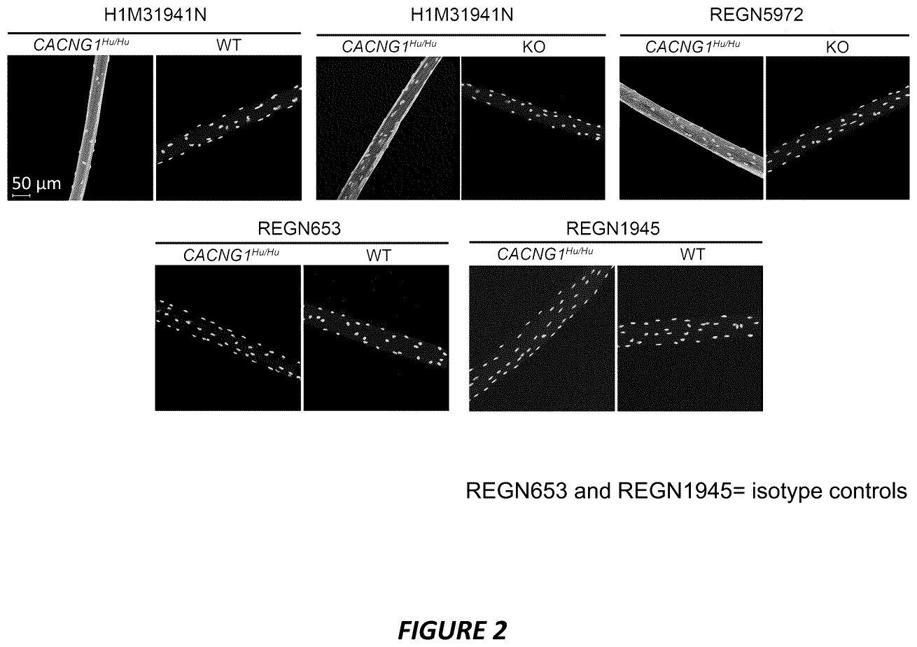

Two CACNG1 antibodies, H1M31941N and REGN5972, demonstrated binding to CACNG1^{Hu/Hu} myofibers ex vivo and did not bind to wildtype or CACNG1 knockout myofibers. Isotype control antibodies did not bind to either CACNG1^{Hu/Hu} myofibers orwildtype myofibers (FIG. 2).

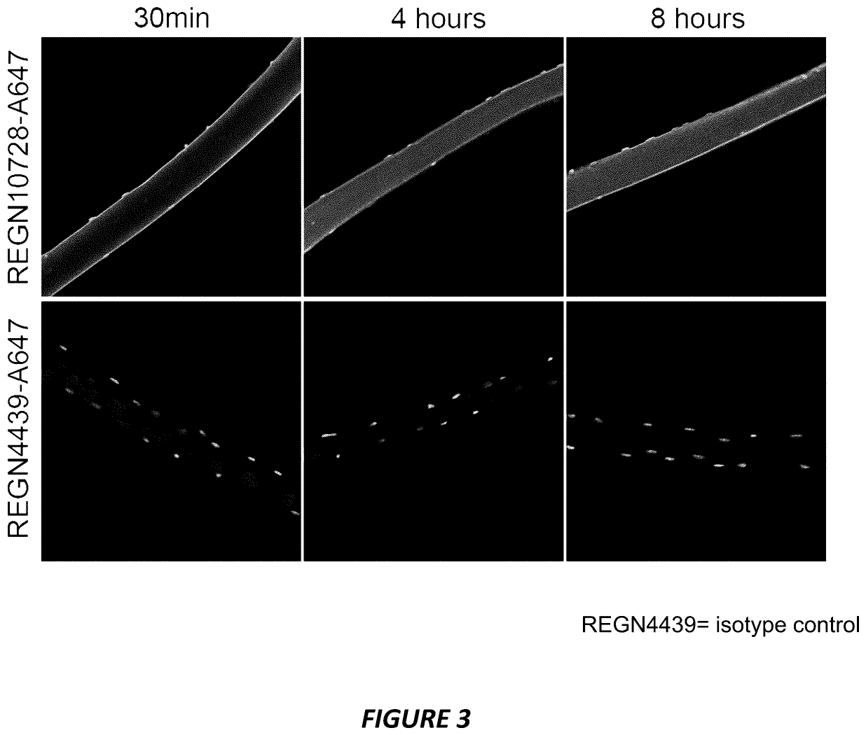

Single plane confocal imaging revealed that the fluorophore-conjugated CACNG1 antibody, REGN10728, bound to the surface of the myofiber following 30 minutes of incubation, and that it was internalized within the myofiber by as early as 4 hours (FIG. 3).

In conclusion, CACNG1 antibodies can bind to the surface of single myofibers ex vivo. Additionally, tracking of a fluorophore conjugated CACNG1 antibody demonstrates initial binding to the surface of the myofiber, followed by internalization into the myofiber after several hours.

### Example 5. CACNG1 Antibody In Vivo Biodistribution

To determine whether CACNG1 antibodies can specifically target skeletal muscle in vivo, CACNG1 antibodies (REGN5972 and REGN10728) and an isotype control antibody (REGN4439) were conjugated with Alexa Fluor 647 fluorescent dye, and were tail vein injected into mice (n=1/group) that were homozygous for the expression of human CACNG1 in place of mouse CACNG1 (referred to as CACNG1^{Hu/Hu}) at a dose of 10 mg/kg, or with saline control. Six days following injection, mice were cryopreserved for whole body antibody distribution analysis using cryo-fluorescence tomography (Invicro). A separate set of mice (n =1/group) that were injected with the same antibodies (or saline control) were sacrificed at 6 days post-injection and PBS perfused, and the following tissues were harvested for immunofluorescence analysis: tibialis anterior, gastrocnemius/plantaris/soleus complex, diaphragm, tongue, pelvic floor muscle, triceps, trapezius, liver, kidney, spleen, brown adipose. Tissues were embedded in optical cutting temperature (OCT) compound, frozen in liquid nitrogen-cooled isopentane, and subsequently cryo-sectioned at 10 μm onto microscope slides. Tissue sections were then permeabilized with Triton X-100, blocked with 4% BSA, and incubated with a rabbit-derived laminin antibody (Sigma) overnight. The following day, sections were washed, stained with anti-rabbit Alexa 488 secondary antibody (Thermo Fisher), counterstained with Hoescht, washed, fixed with 4% PFA, washed, and mounted with Fluoromount-G (Thermo Fisher). Tissues were then imaged on a Zeiss Axioscan Z1 slide scanner to visualize tissue distribution of Alexa 647-conjugated antibodies.

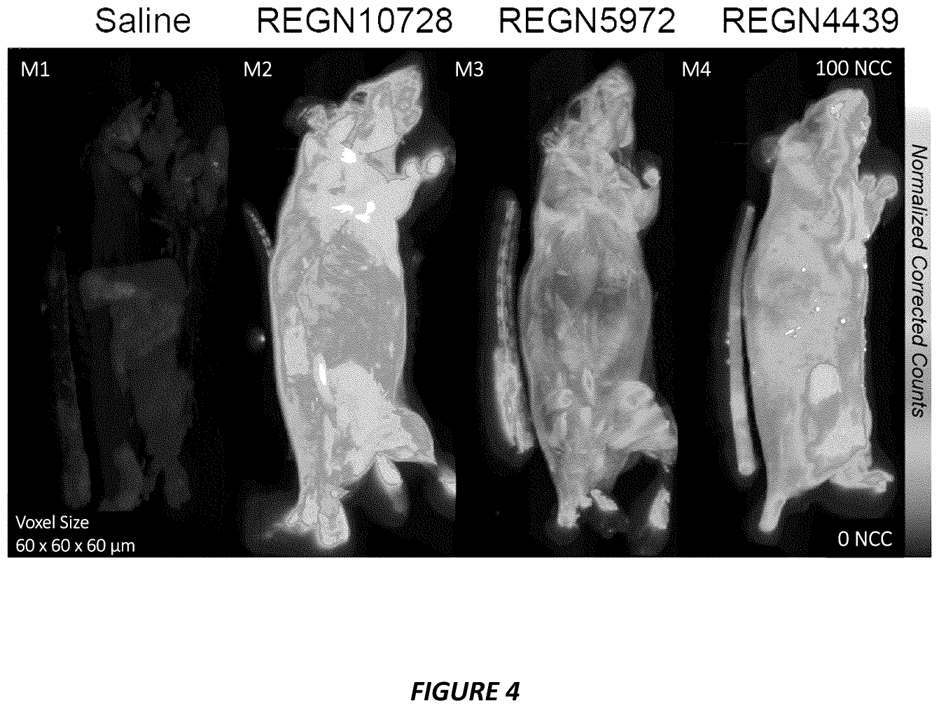

Alexa 647-conjugated CACNG1 antibodies, REGN10728 and REGN5972 displayed clear signal in multiple skeletal muscles via cryo-fluorescence tomography imaging, while the isotype control antibody did not show muscle uptake and accumulated mostly in the bladder (FIG. 4). Saline-dosed control did not show any appreciable fluorescent signal throughout the mouse. Fluorescent signal in the muscle appeared stronger in the mouse dosed with CACNG1 antibody REGN10728 compared to REGN5972, though their overall muscle distribution pattern was similar.

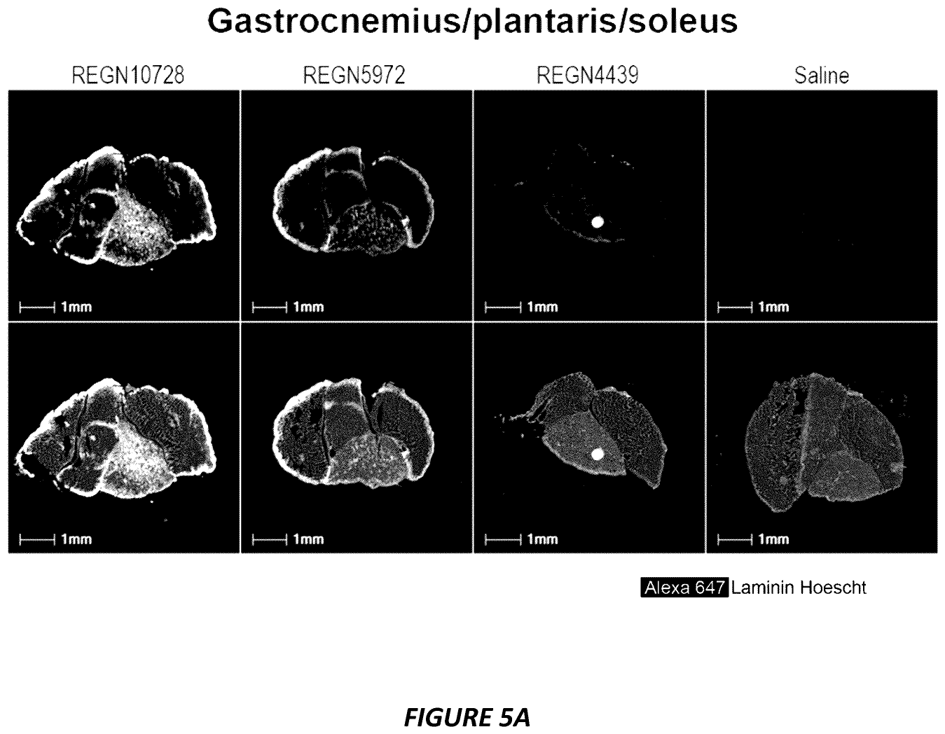

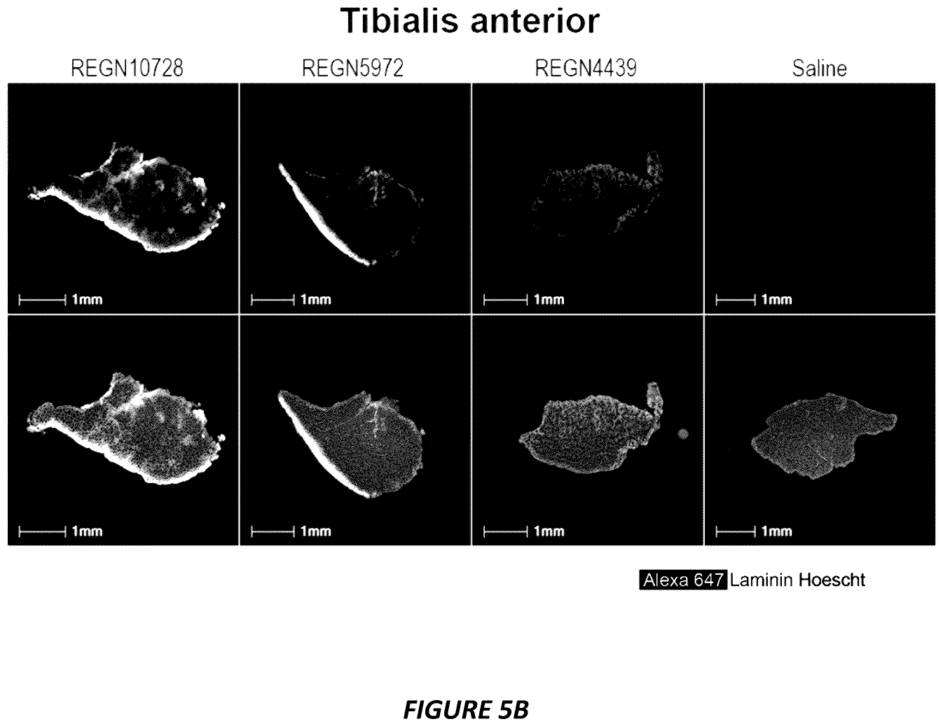

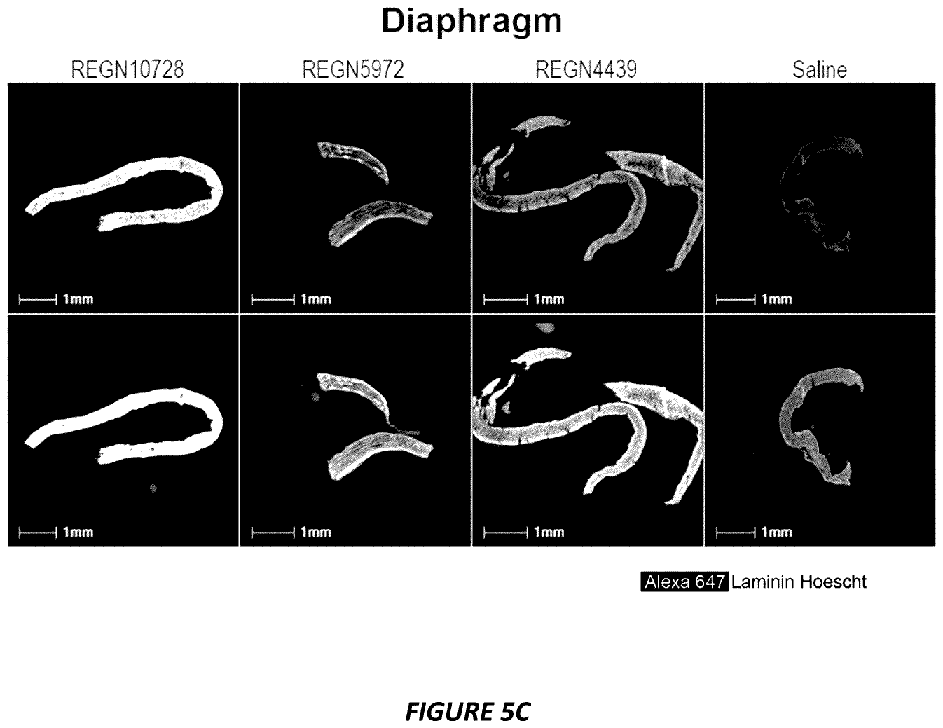

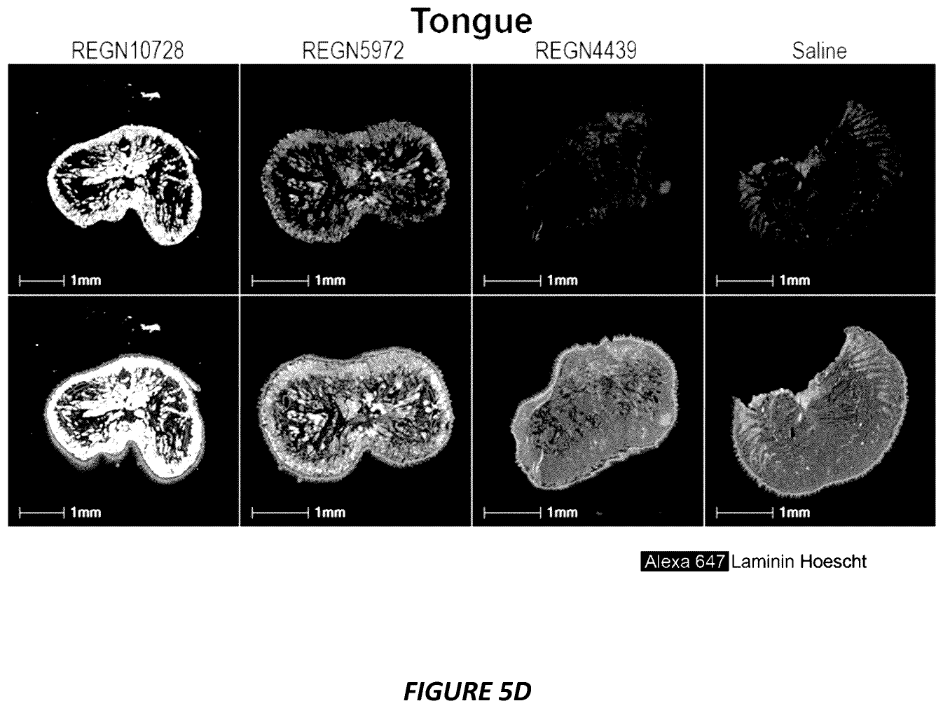

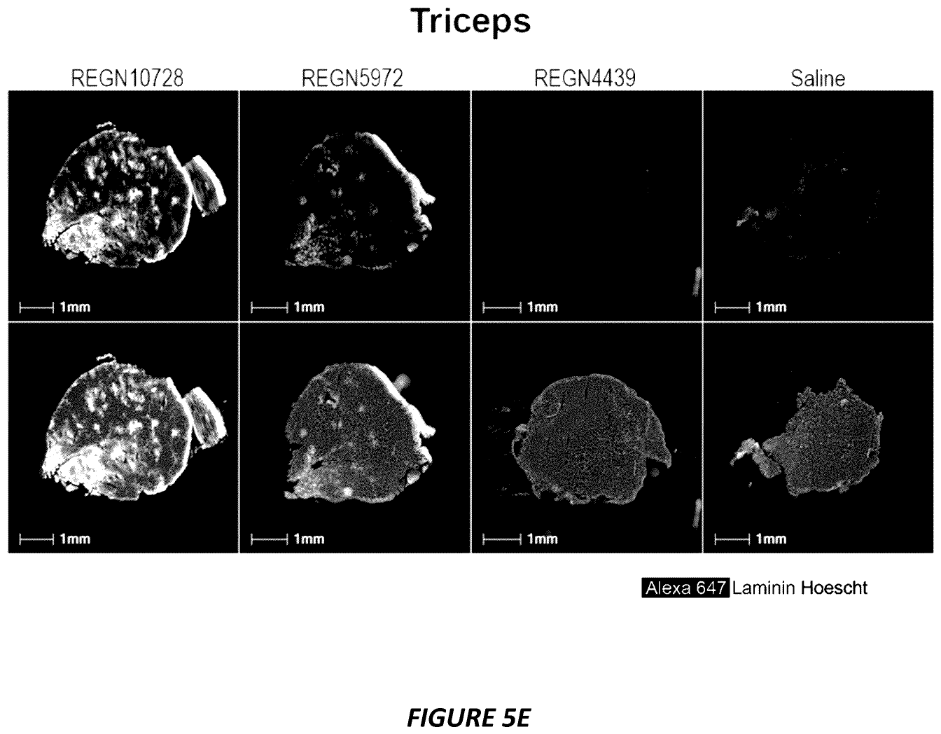

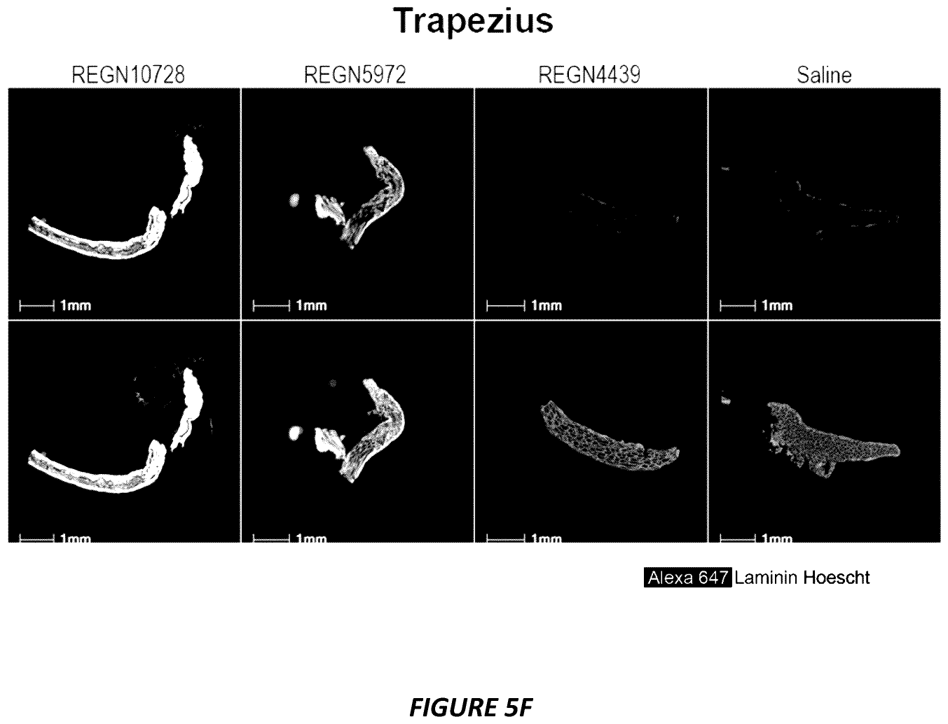

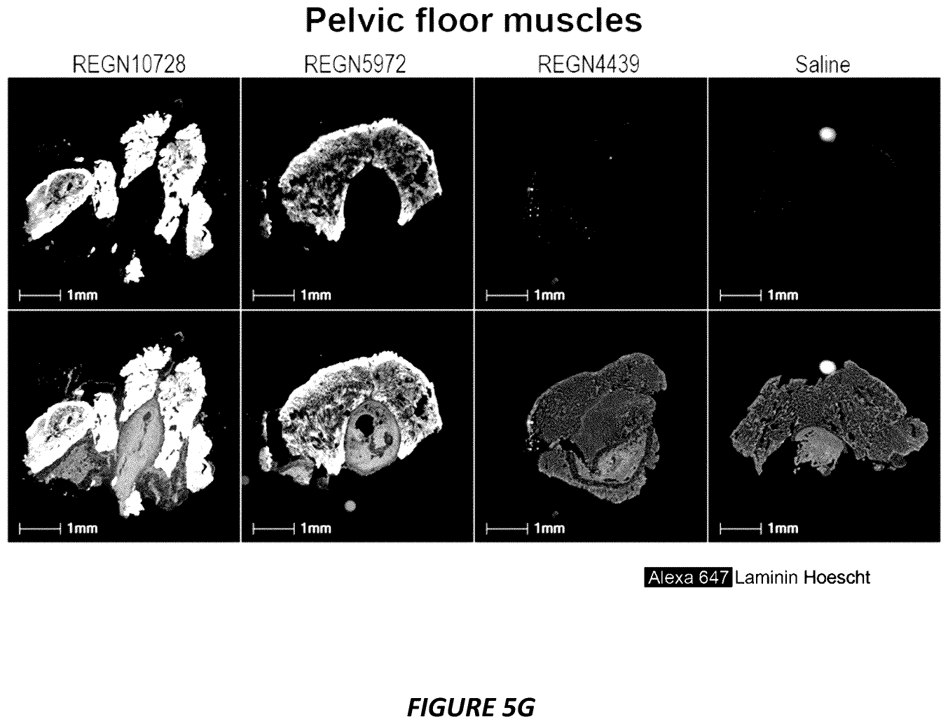

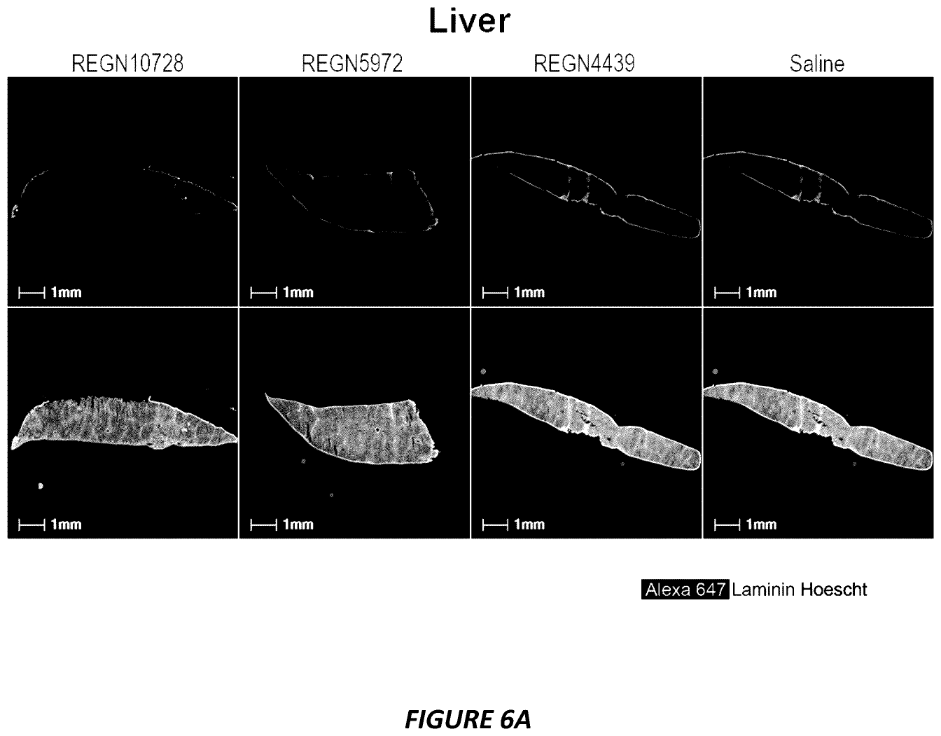

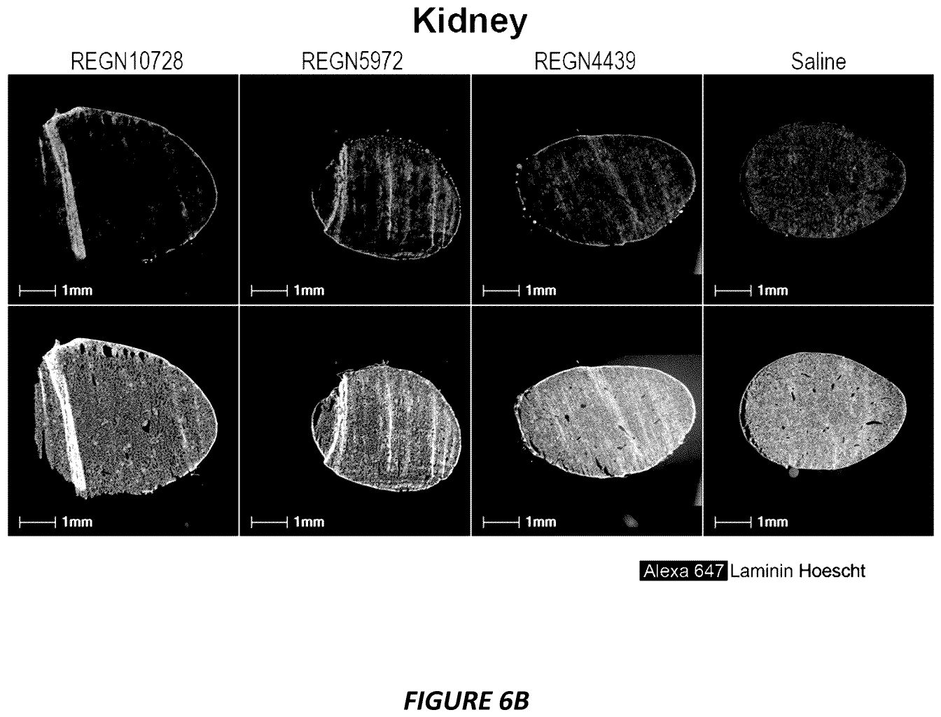

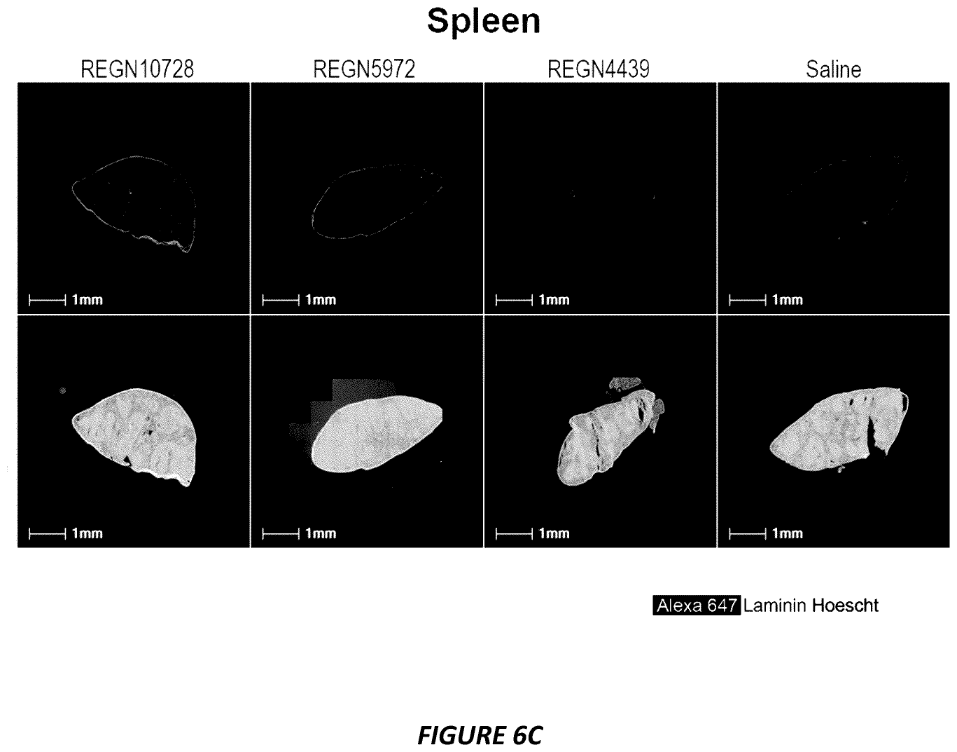

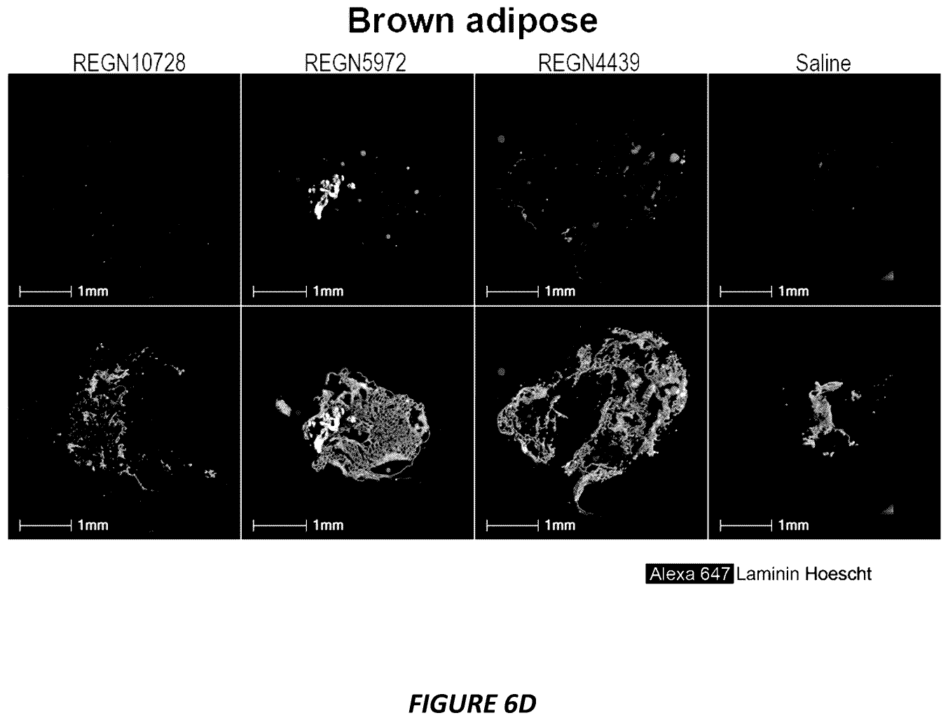

Fluorescence imaging of tissue sections revealed uptake of Alexa 647-conjugated CACNG1 antibodies REGN10728 and REGN5972 in multiple skeletal muscles, including: gastrocnemius/plantaris/soleus complex, tibialis anterior, diaphragm, tongue, triceps, trapezius, and pelvic floor muscles (FIGS. 5A-5G, respectively). Similar to cryo-fluorescence tomography imaging findings, the overall signal of REGN10728 appeared stronger in the muscle sections compared to REGN5972. Neither REGN10728 and REGN9572 showed any clear staining in multiple non-muscle tissues, including: liver, kidney, spleen, and brown adipose (FIGS. 6A-6D, respectively).

In conclusion, in vivo biodistribution studies demonstrated that fluorophore-conjugated CACNG1 antibodies specifically target skeletal muscle, and are not taken up by other non-muscle tissues.

### Example 6. CACNG1 Antibody Distribution to Muscle is Altered by Exercise and Dose

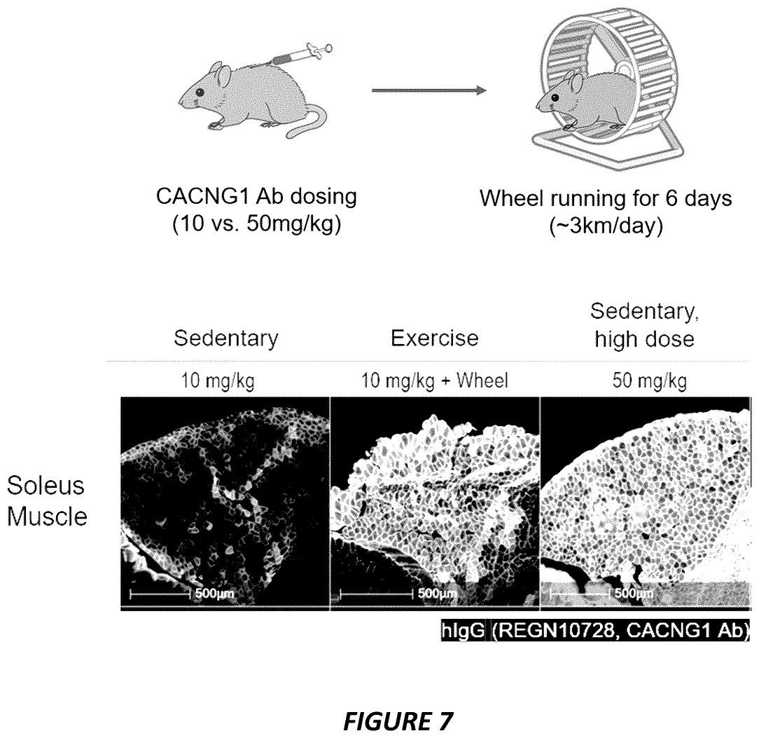

To test methods to enhance CACNG1 antibody distribution to muscle, CACNG1^{Hu/Hu} mice were dosed with 10 mg/kg or 50 mg/kg CACNG1 antibody (REGN10728), and a subset of mice were given access to exercise wheels (FIG. 7, top panel). Six days following dosing, mice were sacrificed, and the gastrocnemius/plantaris/soleus muscle was harvested for immunofluorescence analysis. Tissues were embedded in optimal cutting temperature (OCT) compound, frozen in liquid nitrogen-cooled isopentane, and subsequently cryo-sectioned at 10 μm onto microscope slides. Tissue sections were then permeabilized with Triton X-100, blocked with 4% BSA, and incubated with an anti-hlgG-Alexa 647 antibody (Thermo Fisher) for one hour, and then mounted in Fluoromount-G (Thermo Fisher). Voluntary wheel running (approximately 3 kilometers per 24 hours) enhanced CACNG1 antibody distribution to the working soleus muscle compared to sedentary mice dosed at 10 mg/kg, and 50 mg/kg dose in sedentary mice also showed enhanced distribution throughout the soleus compared to the sedentary 10 mg/kg dosed group (FIG. 7, bottom panel).

### Example 7. In Vitro and Ex Vivo Screening of Purified CACNG1 Antibodies Using Human and Mouse Myotubes

A total of 43 purified CACNG1 antibodies from two immunization campaigns were screened in vitro using human and mouse myotubes. Incubation of live myotubes with CACNG1 antibodies followed by fluorophore-conjugated secondary detection was performed to assess antibody binding. Antibodies were incubated with various concentrations of antibody for 30 minutes, washed, incubated with Alexa647-conjugated F(ab′)2 secondary antibody (Jackson) for 30 minutes, washed, fixed, counterstained for Hoescht, and imaged on a fluorescent microscope. FIG. 10A shows the results for two of the purified CACNG1 antibodies. Live staining of human myotubes with 25 nM of anti-CACNG1 antibody followed by fluorophore-conjugated secondary antibody detection displayed robust binding compared to isotype control (FIG. 10B).

Incubation of myotubes with CACNG1 antibodies followed by Duocarmycin-conjugated secondary was performed to assess antibody internalization via cell kill. Primary human myotubes or C2C12 myotubes were incubated with CACNG1 antibodies and Fab anti-Fc secondary antibody conjugated to Duocarmycin DM (30 nM, Moradec) for 144 hours at 37° C., 5% C02 for 144 hours. Plates were then equilibrated to room temperature for 15 minutes and Cell Titer Glo (Promega) was added and agitated for 10 minutes, and luminescence was read on a plate reader (Envision). Percent viability from this secondary cell kill assay was used to determine internalization, with greater cell kill representing better internalization (see, e.g., FIG. 10C).

Immunostaining for CACNG1 in CACNG1^{Hu/Hu} mouse single myofibers and muscle tissue cross sections (FIG. 10D) confirmed that CACNG1 is expressed at the cell surface of the myofiber.

### Example 8. mRNA Knockdown in Target Tissues Following Systemic Delivery of Antibody-Oligonucleotide Conjugate (AOC)

To enhance skeletal muscle delivery of therapeutic oligonucleotides, skeletal muscle-specific antibodies targeting CACNG1, the γ1 subunit of the skeletal muscle dihydropyridine receptor, are developed. Conjugation of oligonucleotides (e.g., siRNA, ASO) to these CACNG1 antibodies can provide efficient gene knockdown or exon skipping in skeletal muscle for the treatment of muscle diseases. Table 5-1 shows a description of the anti-CACNG1 antibodies for testing in the present Example.

TABLE 5-1
Anti-CACNG1 antibodies for testing

| REGN # | Description |
|---|---|
| REGN14572-M3463- | CACNG1 hIgG1 antibody conjugated |
| siRNA1 | with siRNA |
| REGN3892-M3463- | Isotype control hIgG1 antibody |
| siRNA1 | conjugated with siRNA |
| Control Ab1-M3463- | Isotype control rIgG2a antibody |
| siRNA1 | conjugated with siRNA |
| Control Ab2-M3463- | TfR1 rIgG2a antibody conjugated |
| siRNA1 | with siRNA |

M3463 is a linker as structure below (purchased from Broadpharm, BP-22617)

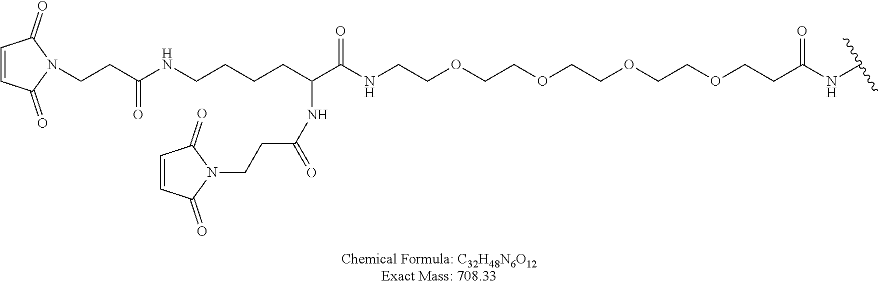 [SMILES: *NC(=O)CCOCCOCCOCCOCCNC(=O)C(CCCCNC(=O)CCN1C(=O)C=CC1=O)NC(=O)CCN1C(=O)C=CC1=O]

CACNG1 antibody is conjugated with siRNA against DM1 Protein Kinase (DMPK) to test antibody-oligonucleotide conjugate-targeted knockdown of DMPK specifically in skeletal muscle in mice. DMPK is a gene that encodes myotonin-protein kinase, a serine/threonine protein kinase. In humans, a CTG repeat expansion in the 3′ non-coding, untranslated region of DMPK is associated with myotonic dystrophy type I (DM1), the most common form of myotonic dystrophy (DM). From a functional standpoint, the repeat expansion of a CTG trinucleotide repeat in DM1 patients may comprise from approximately 50 to 3,000, or more, total repeats thereby leading to generation of toxic RNA repeats capable of forming hairpin structures that bind essential intracellular proteins (e.g., muscleblind-like proteins) with high affinity causing protein sequestration and loss-of-function phenotypes that are characteristic of DM1. There are as yet no effective therapeutics for DM1.

The siRNA against DMPK (siRNA1) comprises the nucleotide sequence that targets the DMPK gene. The control reagent is a non-targeting antibody conjugated with the same siRNA, and the comparator molecules is an antibody against transferrin receptor (TfR1) and its associated isotype control conjugated with siRNA. Seven-week-old C57BL6 male mice are maintained on chow diet and are tail vein injected with under 150 μl of the following reagents: PBS, 3 mg/kg siRNA1, 3 mg/kg Control Ab1-M3463-siRNA1, 0.3 mg/kg Control Ab2-M3463-siRNA1, 1 mg/kg Control Ab2-M3463-siRNA1, 3 mg/kg Control Ab2-M3463-siRNA1, 3 mg/kg REGN3892-M3463-siRNA1, 0.3 mg/kg REGN14572-M3463-siRNA1, 1 mg/kg REGN14572-M3463-siRNA1, and 3 mg/kg REGN14572-M3463-siRNA1. Five mice per group are used for experiments. Bleeds for detection of circulating antibody are performed at baseline (1 week before dosing), 2 hours, 1 day, 4 days and 7 days post-dosing.

Seven days after injection, mice are anesthetized by isoflurane and then sacrificed by cervical dislocation. Gastrocnemius, tibialis anterior, soleus, quadriceps and diaphragm muscles are collected in RNAlater, as well as the heart, liver, spleen, kidney, and lungs. After resting in RNAlater for 4 hours, samples are frozen at −20° C. until RNA isolation.

Fc ELISAs-To determine circulating antibody levels, high binding 96-well clear plates (Thermo Scientific Pierce, catalog #15041) are coated overnight with antibodies against human and rat IgG (Jackson Immunoresearch, Catalog #109-005-098, 112-005-167). Plates are washed, blocked, washed again, and then incubated with diluted serum samples from bleeds described before. Plates are washed again and incubated with antibodies against human and rat IgG conjugated with horseradish peroxidase. Plates are washed and incubated with TMB substrate followed by sulfuric acid to stop the reaction, and then are read on an optical plate reader at 450 nm wavelength.

RNA isolation-RNA is purified from samples in RNAlater using the automated MagMax (Thermo Scientific) protocol.

cDNA synthesis/qPCR-250 ng-1 μg of RNA sample is treated with DNAse I (Thermo Scientific Catalog #EN0521) and then reverse transcribed using the SuperScript VILO Master Mix (Invitrogen Catalog #11755-050). cDNA is diluted 10-fold with nuclease-free H₂O, and then assayed by qPCR. TaqMan assays for Dmpk (Mm00446261_m1) and Rplp0 (Mm00725448_s1) are used to determine gene expression with the TaqMan Gene Expression Master Mix (Applied Biosystems, Catalog #4369106) on the QuantStudio 6 Flex System (Applied Biosystems).

### Example 9. siRNA Synthesis, Coupling of the Linker to siRNA, and Conjugation of the siRNA to the Antibody

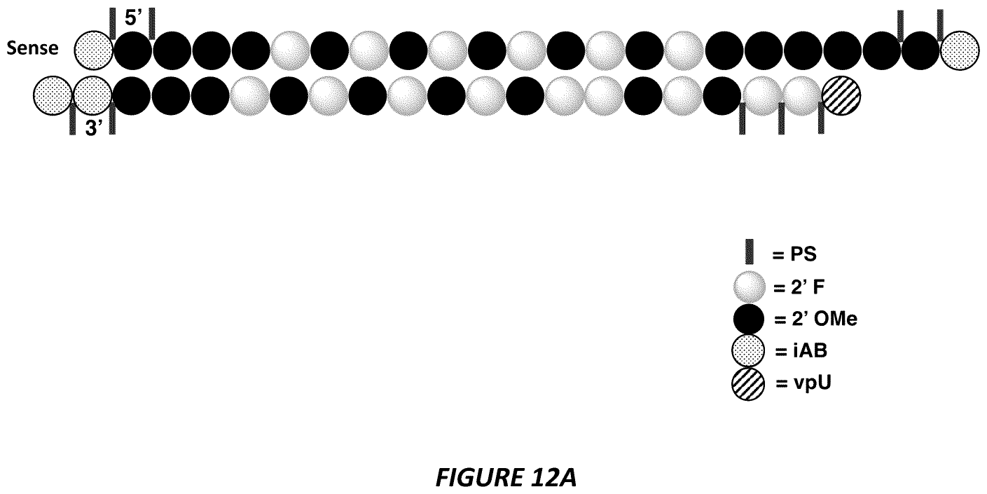

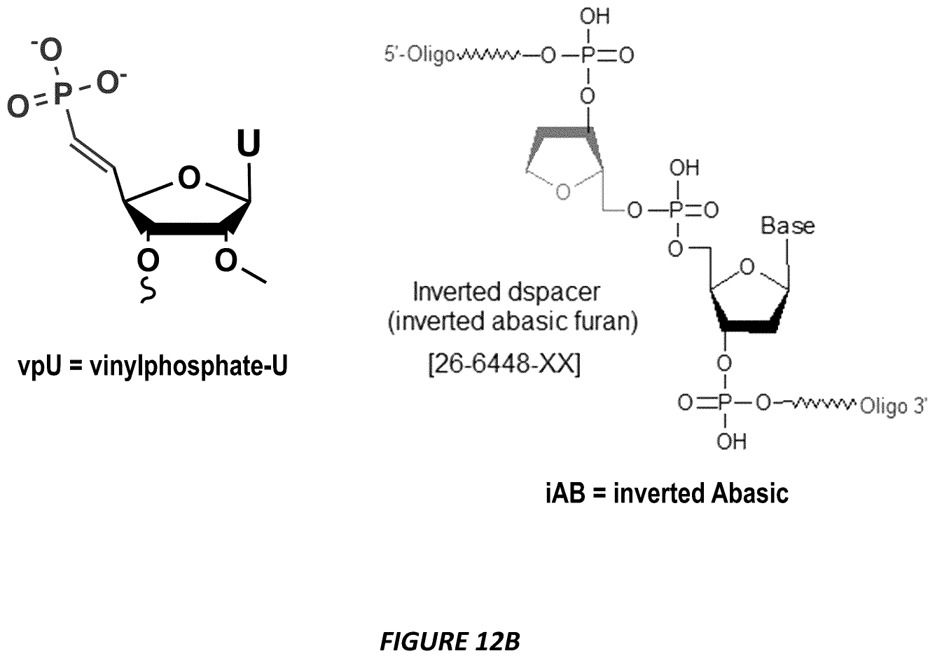

To enhance skeletal muscle delivery of siRNA, skeletal muscle-specific antibodies targeting CACNG1 were conjugated with siRNA against DM1 Protein Kinase (DMPK). The structure of the siRNA against DMPK (siRNA1) is described in FIG. 12. A bifunctional linker (M3463) (purchased from Broadpharm, BP-22617) depicted above (see Example 8) was used as a linker. Table 6-1 shows a description of the antibody-siRNA conjugates generated in the present Example.

TABLE 6-1
Antibody-siRNA conjugates

| REGN # | Description |
|---|---|
| REGN14572-M3463-siRNA1 | Anti-CACNG1 N297Q hIgG1 antibody |
| | conjugated with siRNA |
| REGN3892-M3463-siRNA1 | Isotype control N297Q hIgG1 antibody |
| | conjugated with siRNA |
| Control Ab1-M3463-siRNA1 | Isotype control rIgG2a antibody |
| | conjugated with siRNA |
| Control Ab2-M3463-siRNA1 | Anti-TfR1 rIgG2a antibody conjugated |
| | with siRNA |

### Step 1: Synthesis of siRNA

The modified single strands were synthesized by standard automated oligonucleotide synthesis. During the on-bead synthesis, a 5′ sense strand modification with a six carbon chain terminating with a primary amino group was attached to the abasic group to provide a handle for the linker (FIGS. 12A-12B; bifunctional linker M3463 depicted above, see Example 8). For siRNA1, the nucleotide sequence for the sense strand (5′-3′) was M3463-(NH)(iAB)*c*cuc(Gf)g(Uf)a(Uf)u(Uf)a(Uf)u(Gf)ucuau*u*(iAB) (SEQ ID NO: 421) and the nucleotide sequence for the antisense strand was (vpu)*(Af)*(Gf)*a(Cf)a(Af)(Uf)a(Af)a(Uf)a(Cf)c(Gf)agg*(iAB)*(iAB) (SEQ ID NO: 422), wherein * is phosphorothioate (PS), Xf is 2′-fluoro (2′F), x is 2′-O-methyl (2′ OMe) iAB is inverted basic and vpU is =vinylphosphate-U (see, e.g., FIG. 12B). Standard deprotection and cleavage of the strands followed by reverse phase high-performance liquid chromatography (HPLC) purification furnished the strands that were annealed to form the duplex siRNA.

### Step 2: Coupling of the Linker to siRNA

A bifunctional linker (M3463) depicted above (see Example 8) was coupled to the terminal amino group of the 5′ sense strand modification using an excess of the linker. The mixture was then purified by reverse phase HPLC to furnish the functionalized duplex siRNA ready for antibody conjugation.

### Step 3: Conjugating siRNA to Antibody

The antibody was treated with 1 mM dithiothreitol or TCEP (tris(2-carboxyethyl)phosphine) at 37° C. for 30 minutes. After gel filtration (G-25, pH 4.5 sodium acetate), the bis-maleimido linker siRNA was added to the reduced antibody and the mixture adjusted to pH 7.0 with 1 M HEPES (pH 7.4). After 1 hour the conjugates were purified by size exclusion chromatography and sterile filtered. Protein and linker payload concentrations were determined by UV spectral analysis. Size-exclusion HPLC established that all conjugates used were >95% monomeric, and reversed-phase high-performance liquid chromatography (RP-HPLC) established that there was <0.5% unconjugated linker payload. UV (Hamblett, et al, Cancer Res 2004 10 7063) and hydrophobic interaction chromatography (HIC) determined the loadings to be 1-1.6 siRNAs/antibody.

### Example 10. Anti-CACNG1-DMPK siRNA Conjugates Knockdown DMPK in Skeletal Muscle

An anti-CACNG1 antibody was conjugated with siRNA against DM1 Protein Kinase (DMPK) to test in vivo antibody-oligonucleotide conjugate-targeted specific knockdown of DMPK in skeletal muscle of mice. Table 7-1 shows a description of the antibody-siRNA conjugates tested in the present Example.

TABLE 7-1
Antibody-siRNA conjugates

| REGN # | Description |
|---|---|
| REGN14572-M3463-siRNA1 | Anti-CACNG1 N297Q hIgG1 antibody |
| | conjugated with siRNA |
| REGN3892-M3463-siRNA1 | Isotype control N297Q hIgG1 antibody |
| | conjugated with siRNA |
| Control Ab1-M3463-siRNA1 | Isotype control rIgG2a antibody |
| | conjugated with siRNA |
| Control Ab2-M3463-siRNA1 | Anti-TfR1 rIgG2a antibody conjugated |
| | with siRNA |

The sense and antisense strand of the siRNA against DMPK (siRNA1) comprised the nucleotide sequences CCUCGGUAUUUAUUGUCUA (sense) (SEQ ID NO: 416) and UAGACAAUAAAUACCGAGG (antisense) (SEQ ID NO: 417), respectively (see also, e.g., FIG. 12A-12B). M3463 (purchased from Broadpharm, BP-22617) depicted above (see Example 8) was used as a linker. The control reagent was a non-targeting antibody conjugated with the same siRNA, and the comparator molecules were an antibody against transferrin receptor (TfR1) and its associated isotype control conjugated with siRNA (BioXCell anti-TfR1 BE0175, Lot #821022M2; isotype control Rat IgG2a Lot #815022J1). All antibodies were reduced by TCEP (tris(2-carboxyethyl)phosphine) and then conjugated to the siRNA against DMPK (siRNA1) through cysteine maleimide chemistry. Anti-CACNG1 antibody siRNA drug conjugates were synthesized in accordance with the exemplary protocol described below; other conjugates can follow the same protocol.

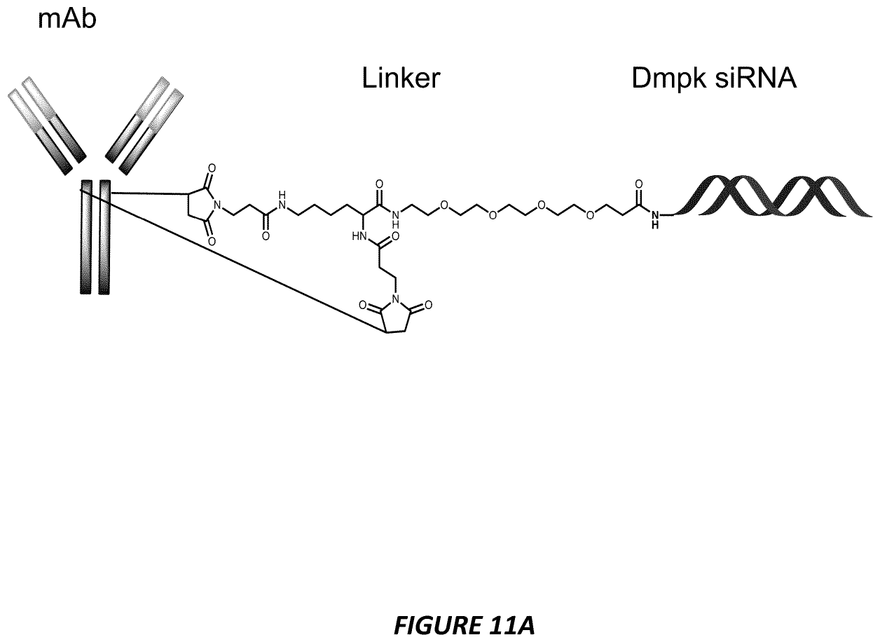

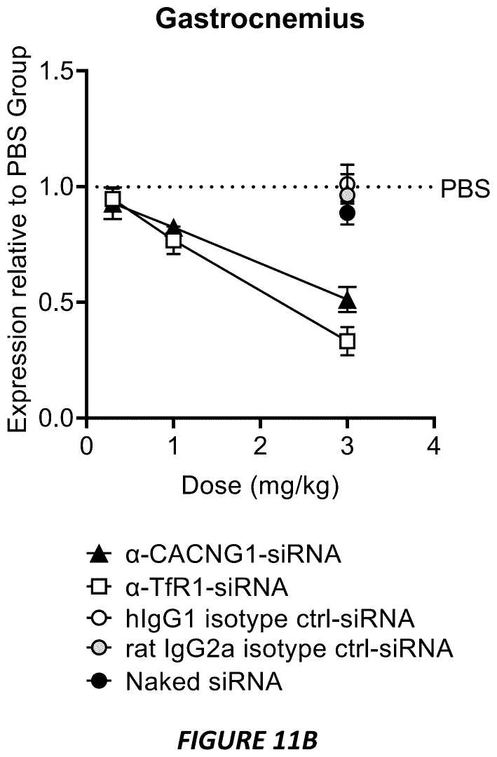

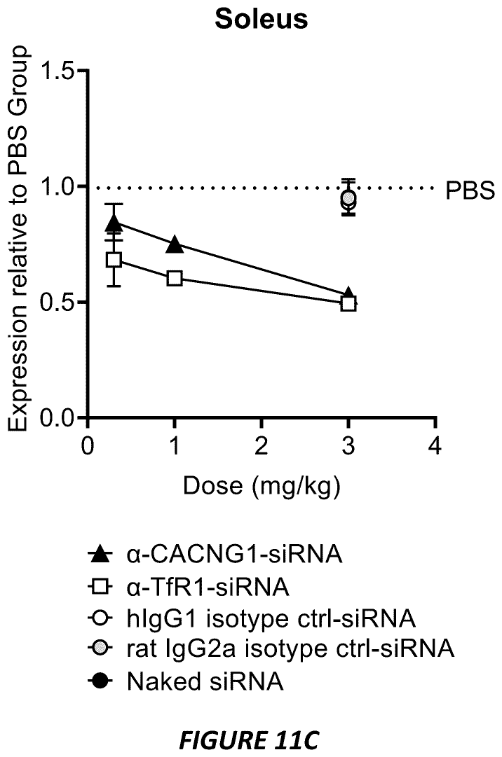

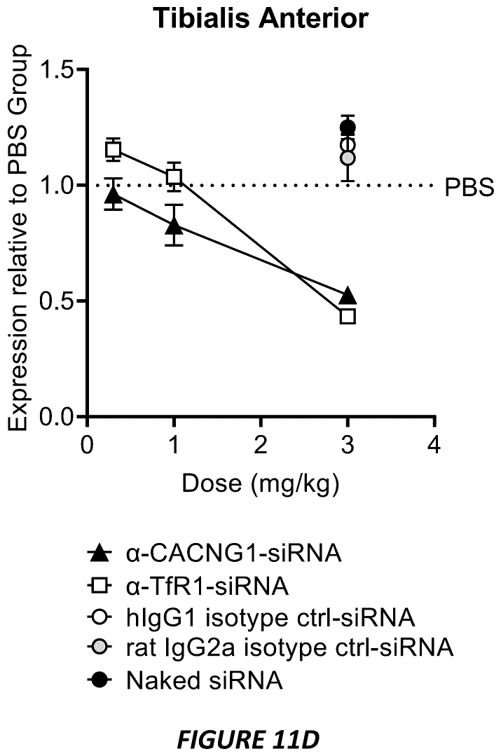

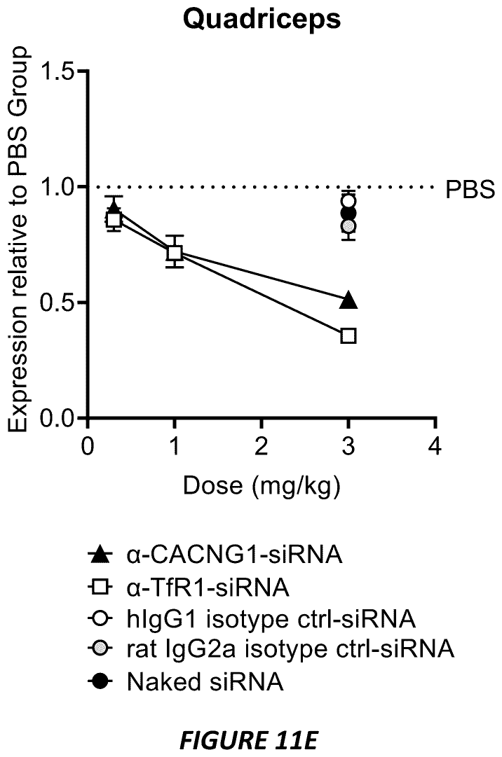

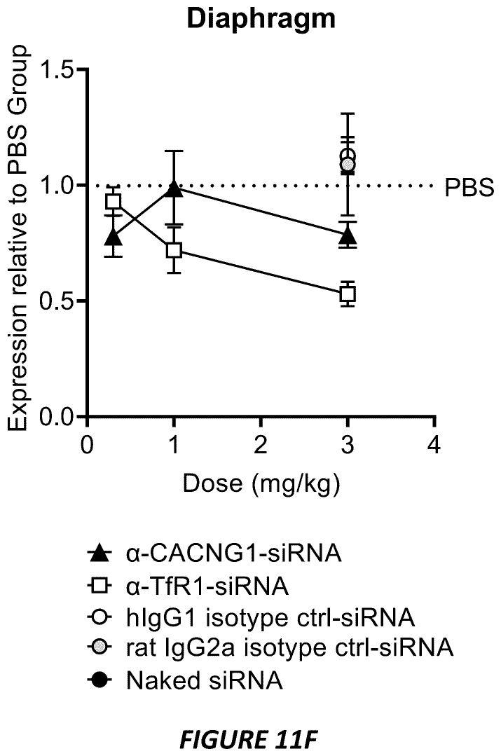

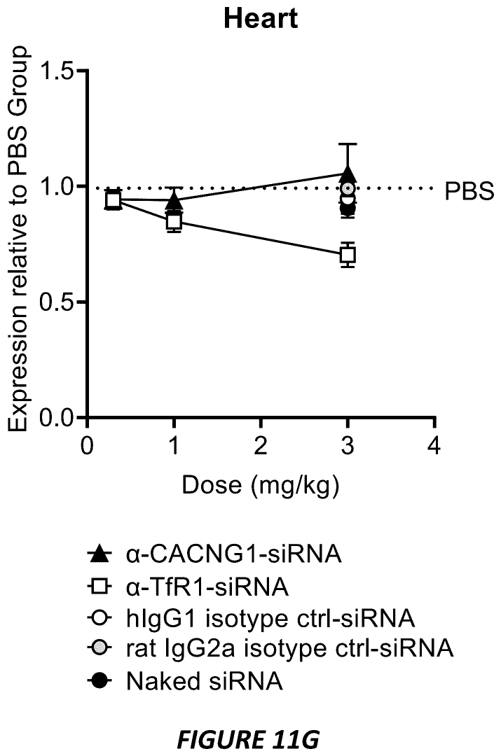

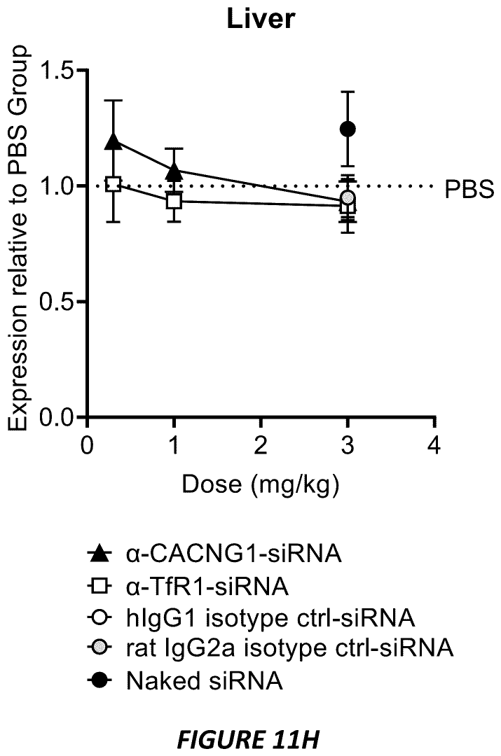

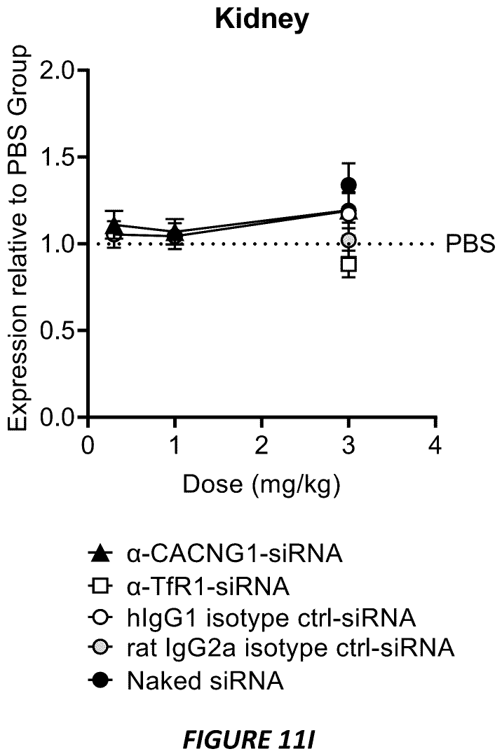

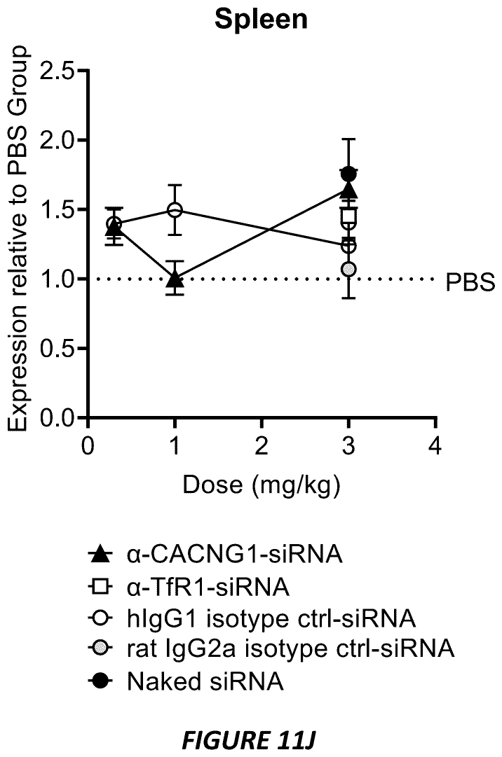

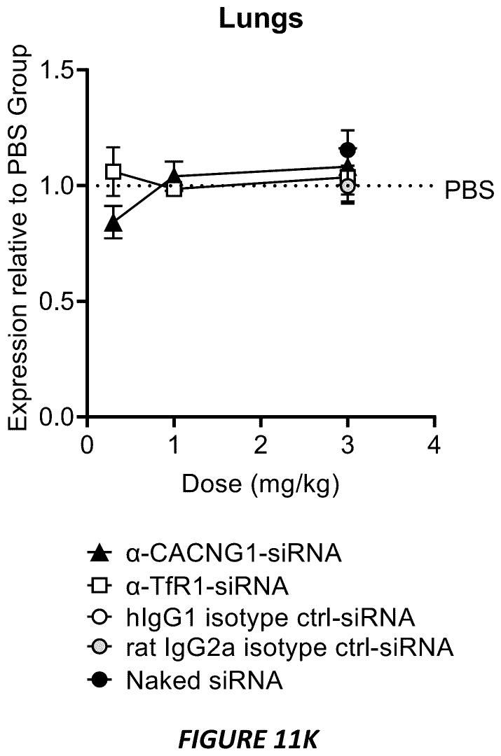

Anti-CACNG mAb REGN14572 was diluted in DPBS, and 50 mM EDTA was added to the sample, followed by 4 mol/mol TCEP. The reaction mixture was incubated at 25° C., 350 RPM, overnight. Three equivalents of M3463-siRNA1 were added and the reaction was incubated at 25° C., 350 RPM, overnight. The crude mixture was purified by AKTA Avant 25 (Model number: 28930842) size exclusion chromatography (Superdex 200 16/600 GL) using Mobile phase: DPBS #2192437 (—CaCl₂, —MgCl₂) and sterile filtered. Protein concentrations and payload-to-antibody ratios were determined by ultraviolet (UV) spectral analysis and HIC (hydrophobic interaction chromatography). Size-exclusion high-performance liquid chromatography (HPLC) testing established that all conjugates were >97% monomeric. Naked siRNA was also used as a control. A schematic representation of an exemplary anti-CAGNG1-DMPK siRNA conjugate is shown in FIG. 11A.

ADCs were further characterized by liquid chromatography electrospray ionization mass spectrometry (LC-ESI-MS) to calculate the drug-to-antibody (DAR) ratio as described herein. The DAR ratios for ADCs of the present Example were as follows: Control Ab1-M3463-siRNA1 (Rat IgG2a isotype control with siRNA): 1.17; Control Ab2-M3463-siRNA1 (anti-TfR1 with siRNA): 1.58; REGN3892-M3463-siRNA1 (anti-FelD N297Q hlgG1 Lot 139 with siRNA): 1.6; and REGN14572-M3463-siRNA1 (anti-CACNG1 N297Q hlgG1 Lot 1): 1.3. All doses in the present Example were adjusted for DAR, i.e., mice were dosed based on siRNA amount for body weight.

Seven-week-old C57BL6 male mice were maintained on chow diet and were tail vein injected with 0.3 mg/kg, 1 mg/kg, or 3 mg/kg total siRNA at antibody-siRNA conjugate doses of approximately 3 mg/kg, 10 mg/kg, or 30 mg/kg, respectively. Five mice per group were used for the experiments.

Seven days after injection, mice were anesthetized by isoflurane and then sacrificed by cervical dislocation. Skeletal muscles, e.g., gastrocnemius, tibialis anterior, soleus, quadriceps and diaphragm muscles, as well as other tissues, including, e.g., heart, liver, spleen, kidney, and lungs were collected in RNAlater. After incubation in RNAlater for 4 hours, samples were frozen at −20° C. until RNA isolation.

DMPK mRNA expression levels (relative to PBS) measured in gastrocnemius, soleus, tibialis anterior, quadriceps, diaphragm, heart, liver, kidney, spleen, and lungs at 0.3 mg/kg, 1 mg/kg, or 3 mg/kg total siRNA are shown in FIGS. 11B-11K, respectively. These data illustrate similar skeletal muscle knockdown of DMPK mRNA at a dose of 3 mg/kg total siRNA for anti-TfR1-siRNA versus anti-CACNG1-siRNA conjugates.

RNA isolation-RNA was purified from samples in RNAlater using the automated MagMax (Thermo Scientific) protocol.

cDNA synthesis/qPCR-50 ng-1 μg of RNA sample was treated with DNAse I (Thermo Scientific Catalog #EN0521) and then reverse transcribed using the SuperScript VILO Master Mix (Invitrogen Catalog #11755-050). cDNA was diluted 10-fold with nuclease-free H₂O, and then assayed by qPCR. TaqMan assays for Dmpk (Mm00446261_m1) and Rplp0 (forward primer, AAGGCCGTGGTGCTGATG (SEQ ID NO: 418); reverse primer, TCTCCAGAGCTGGGTTGTTCT (SEQ ID NO: 419); probe sequence, AAGAACACCATGATGCGCAAGGC (SEQ ID NO: 420) were used to determine gene expression with the TaqMan Gene Expression Master Mix (Applied Biosystems, Catalog #4369106) on the QuantStudio 6 Flex System (Applied Biosystems).

### Example 11. CACNG1 In Vivo siRNA Durability

In vivo delivery of unmodified antisense oligonucleotides (ASOs) and siRNA do not efficiently target skeletal muscle; thus requiring conjugation of targeting moieties to improve delivery to muscle. One strategy to improve oligonucleotide delivery to muscle is by conjugating an antibody that specifically targets skeletal myofibers. Dmpk siRNA (siRNA1) was synthesized and conjugated to CACNG1 antibody REGN14572, which specifically targets skeletal muscle.

To assess the durability of Dmpk knockdown, C57BL/6 mice were tail vein injected with 3 or 5 mg/kg of siRNA conjugated to either CACNG1 Ab (REGN14572-M3463) or isotype control Ab (REGN3892-M3463) or injected with naked siRNA (n=5 per group). All doses were adjusted for drug-to-antibody (DAR) ratio, i.e., mice were dosed based on siRNA amount for body weight, as determined by liquid chromatography electrospray ionization approaches described herein. The DAR ratios for ADCs of the present Example were as follows: REGN3892-M3463-siRNA1 (anti-Feld N297Q Lot 139): 2; and REGN14572-M3463-siRNA1 (anti-CACNG1 N297Q hlgG1 Lot 6): 2. Mice were sacrificed at either 1 week, 3 weeks, or 6 weeks post dosing. The 5 mg/kg group was only assessed at 1 week post dosing. The following tissues were placed into RNAlater (ThermoFisher Catalog #AM7021): gastrocnemius, soleus, quadricep, tibialis anterior, diaphragm, heart, liver, spleen, kidney. Tissues were allowed to sit in RNAlater for at least 2 hours at room temperature before being transferred to −20° C. Total RNA was purified using MagMAX™-96 for Microarrays Total RNA Isolation Kit Catalog #AM1839 (Ambion by Life Technologies) according to manufacturer's specifications. DNAse I treatment of samples (Thermo Scientific Catalog #EN0525) was used as per manufacturer's specifications for samples that had enough RNA for a final concentration of 500 ng, otherwise samples did not get DNase I treatment. Samples were then reverse transcribed using SuperScript VILO MasterMix (Invitrogen Catalog #11755-250) according to manufacturer's specifications. For TaqMan gene expression analysis, TaqMan Gene Expression MasterMix (ThermoFisher Scientific Ref #4369016) was used according to manufacturer's specifications. The probes used in the assay were as followed: Dmpk FAM (ThermoFisher Mm00446261_m1) and Rplp0 (ThermoFisher Mm01974474_gH). Samples were loaded into a MicroAmp 384-well plate (ThermoFisher Scientific Catalog #4309849) and ran on the QuantStudio 6 Flex system using the standard TaqMan protocol with results being analyzed using ΔΔCt method.

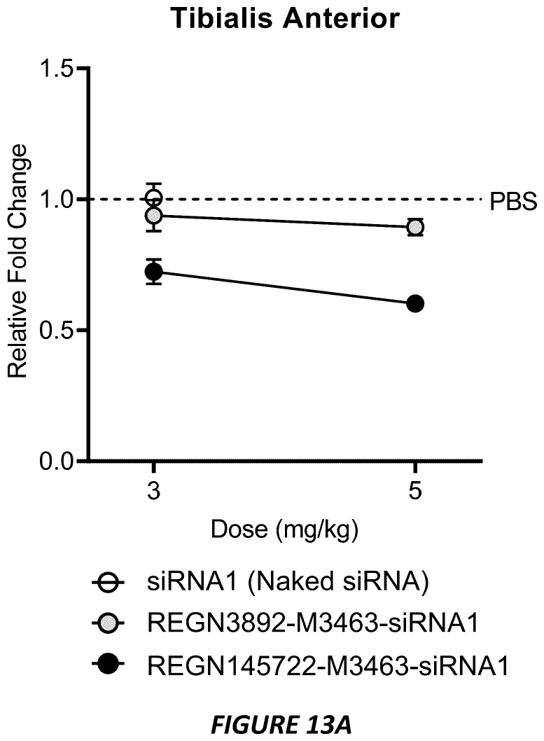

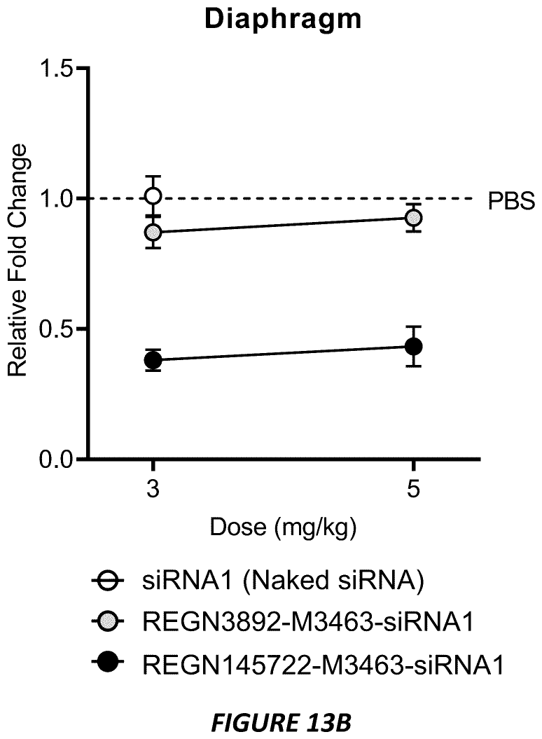

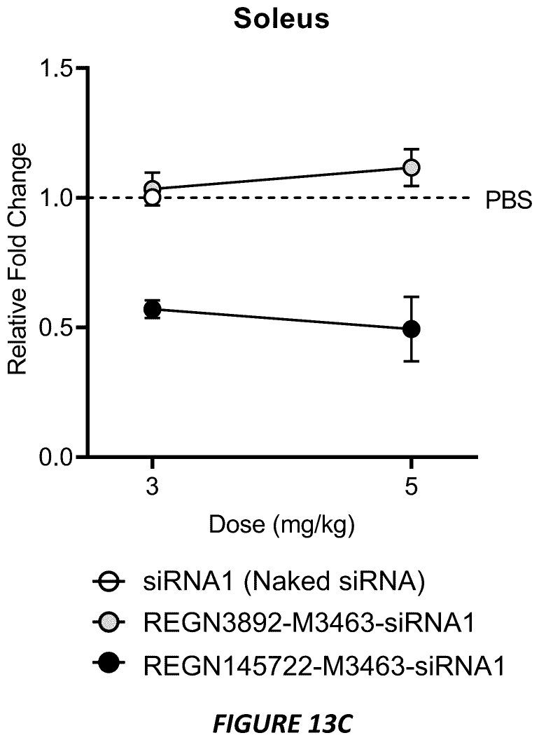

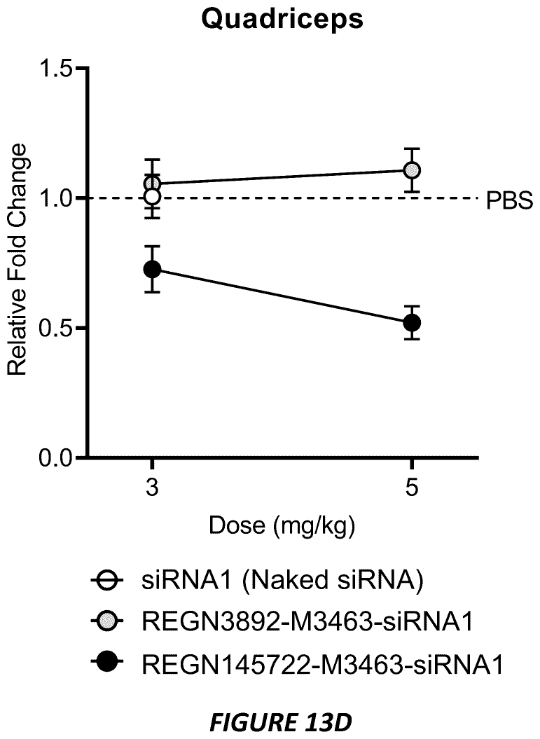

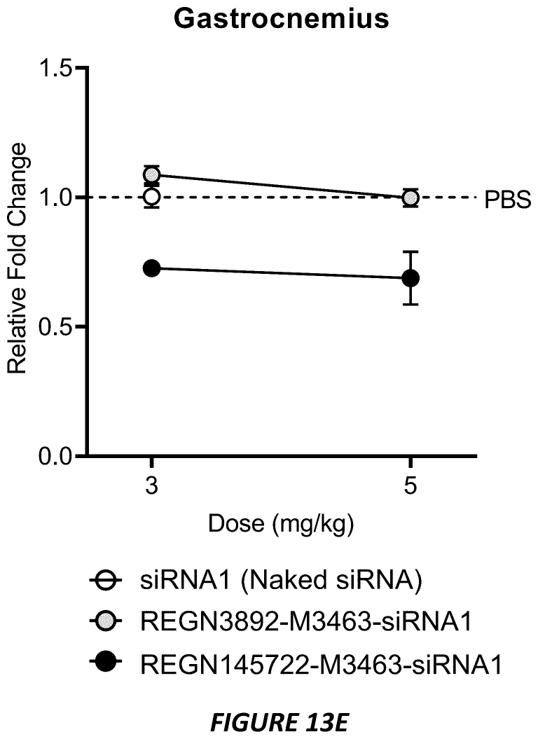

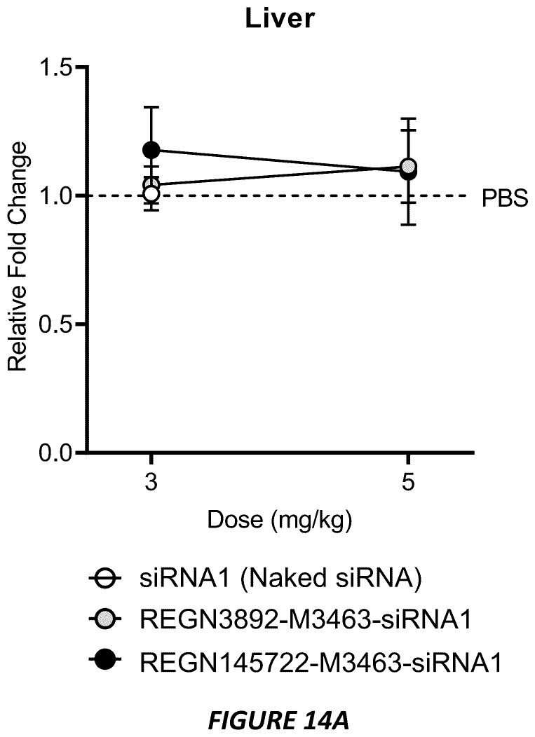

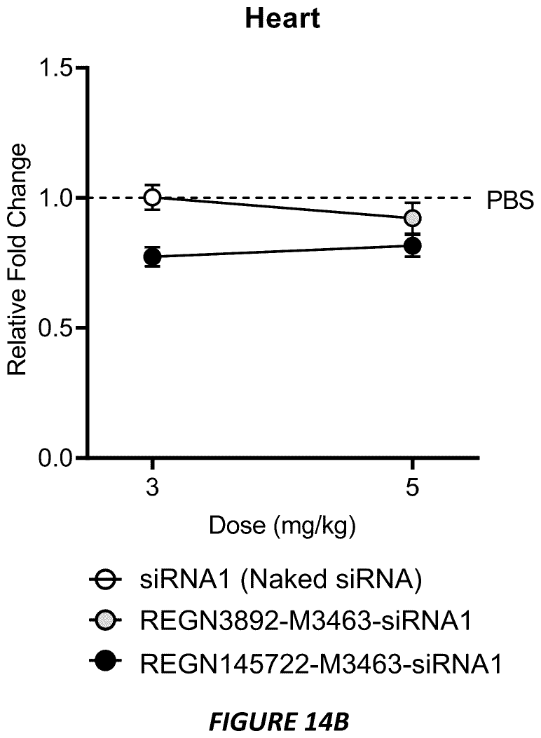

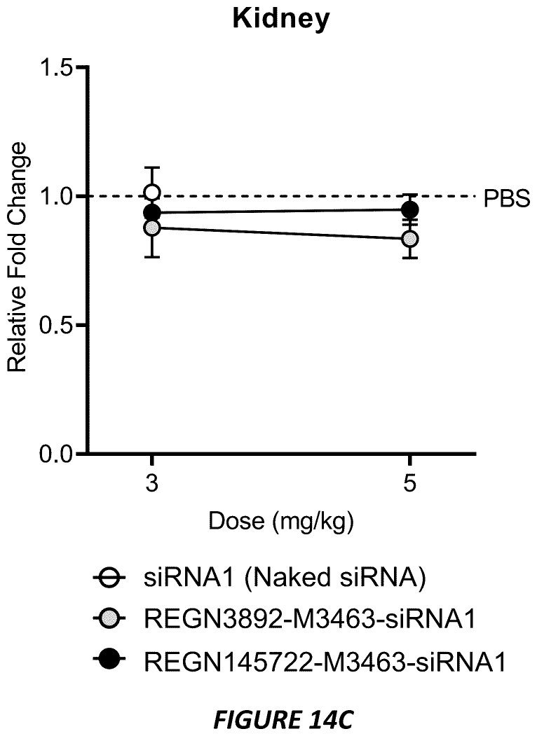

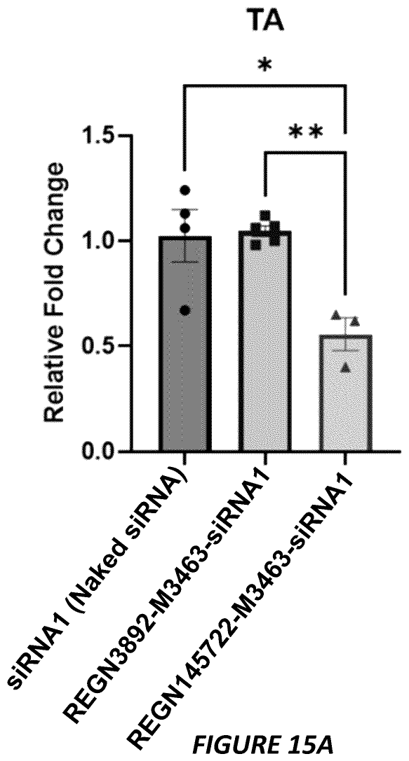

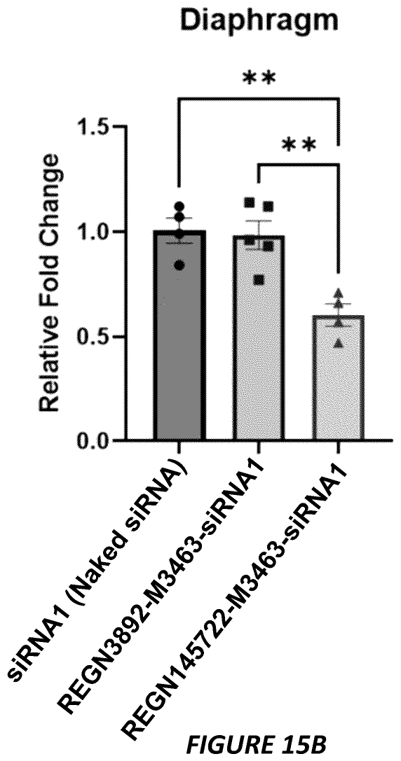

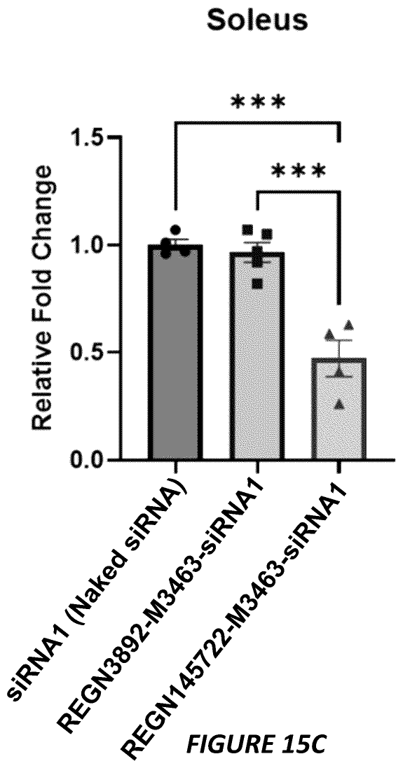

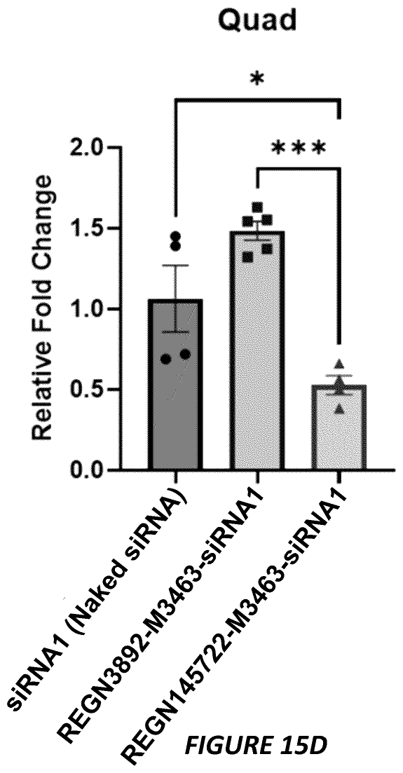

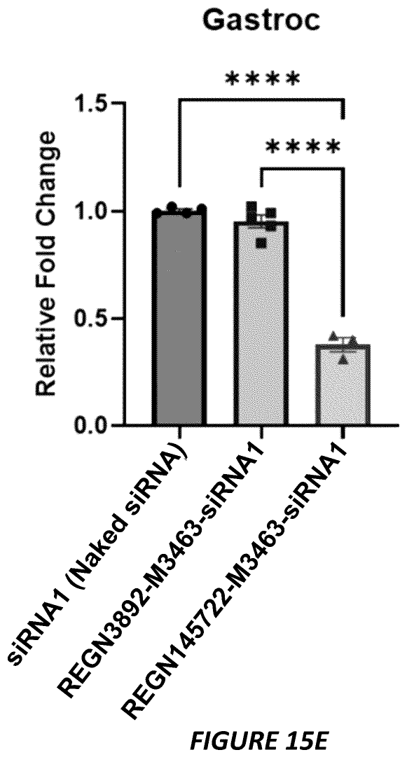

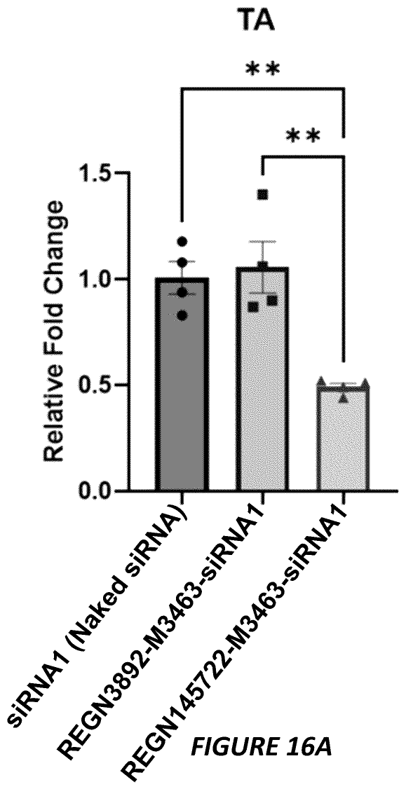

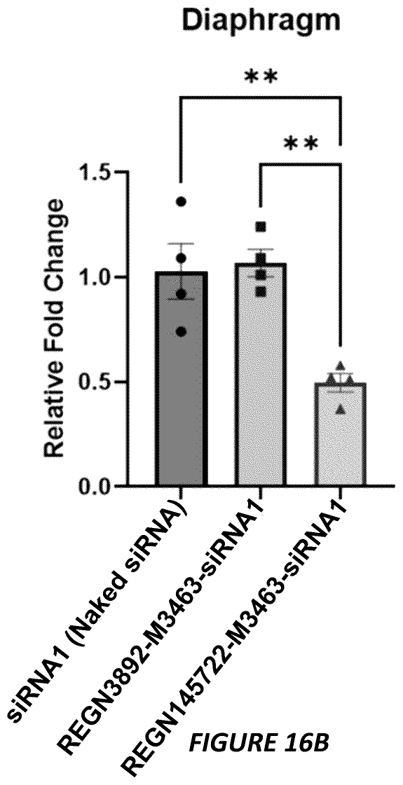

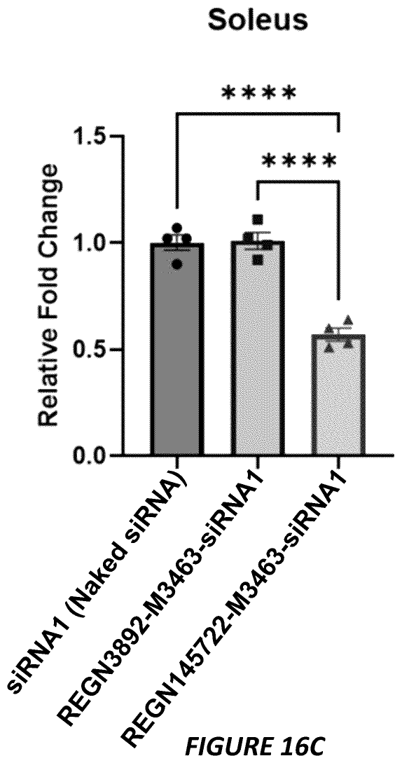

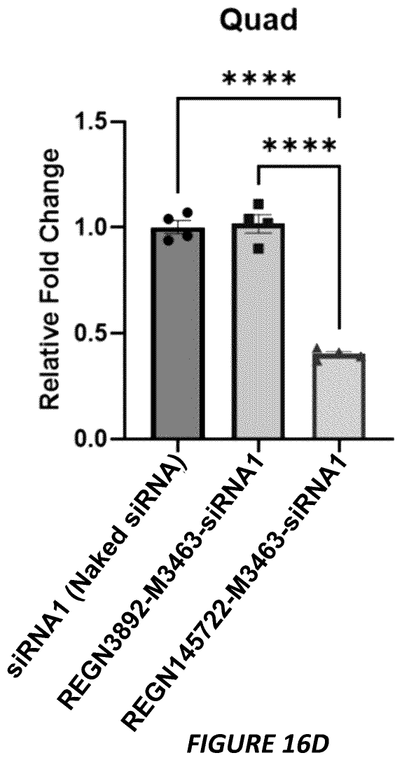

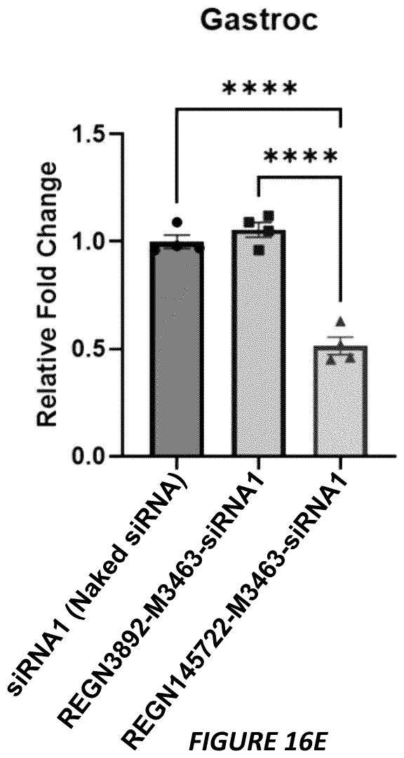

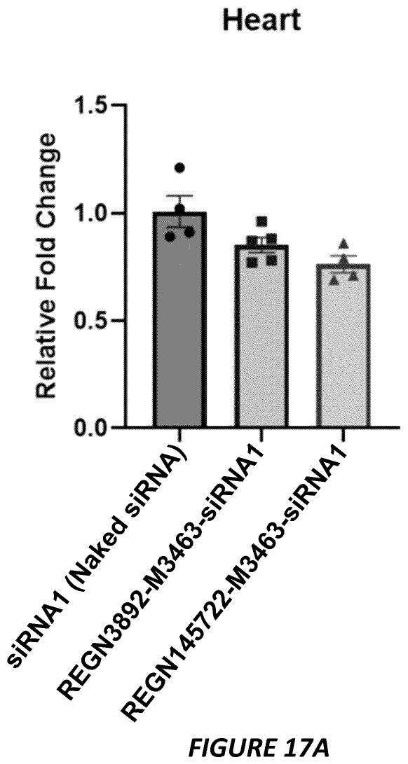

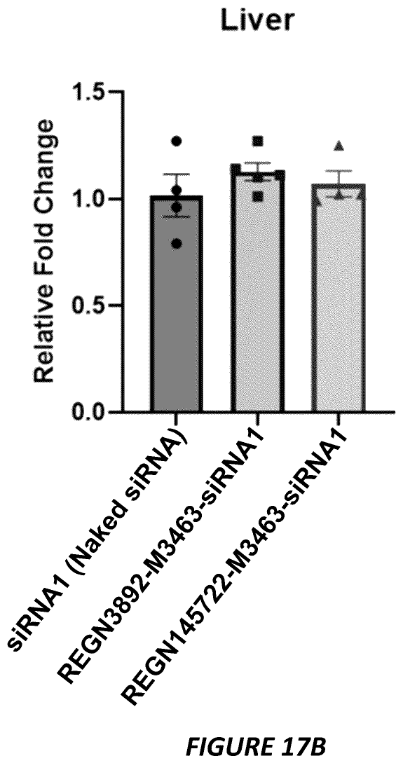

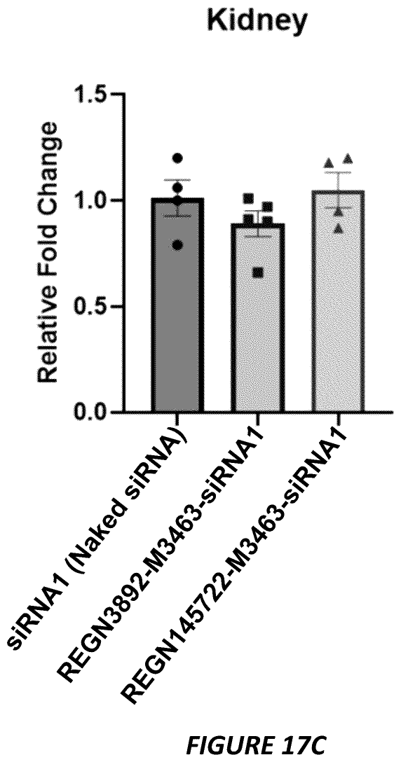

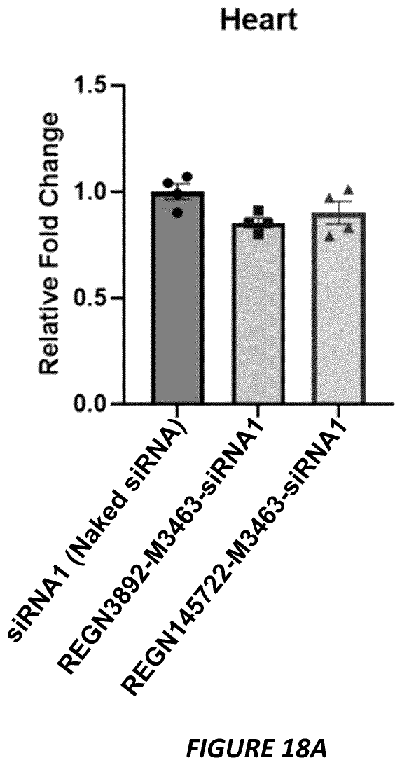

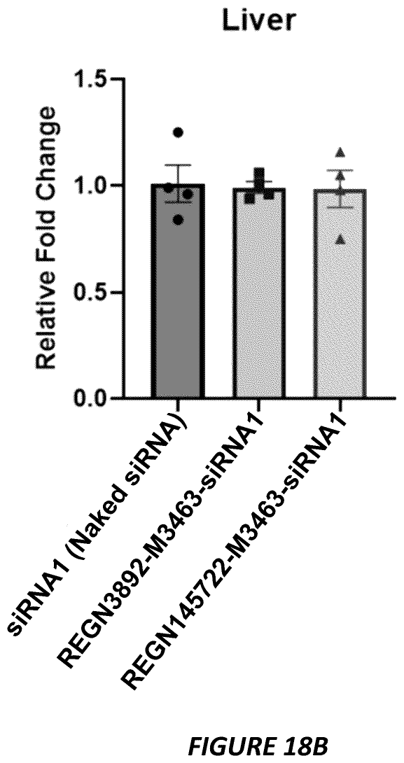

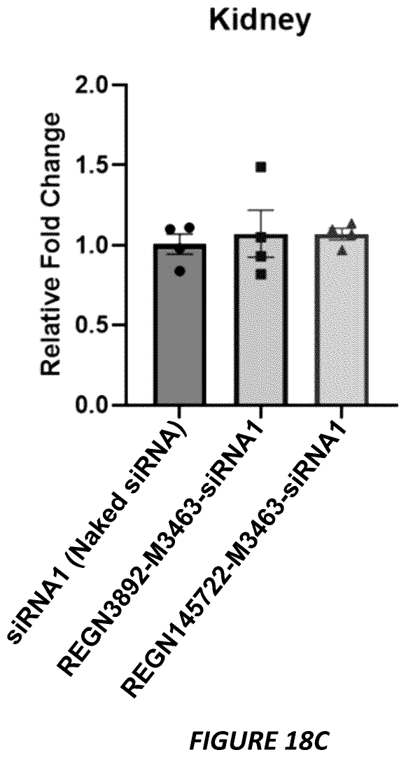

After 1 week post treatment there was a significant reduction of Dmpk expression in skeletal muscle (˜30-60% knockdown) at the 3 and 5 mg/kg dose using a-CACNG1-Dmpk siRNA conjugate (REGN14572-M3463), compared to no noticeable knockdown with isotype control conjugated Ab or naked siRNA (FIGS. 13A-13E). This knockdown was observed only in skeletal muscle, as other tissues did not display any knockdown at either dose (FIGS. 14A-14C). Skeletal muscle knockdown was durable at least 6 weeks post dosing, with a ˜45-60% reduction in Dmpk expression with REGN14572-M3464 at 3 weeks (FIGS. 15A-15E) and 6 weeks (FIGS. 16A-16E) (see also Table 8-1). Similar to 1 week post dosing, there was no knockdown in other organs at 3 weeks (FIG. 17A-17C) and 6 weeks (FIGS. 18A-18C) (see also Table 8-1).

TABLE 8-1
Dmpk expression relative to naked siRNA at 3 and 6 weeks post dosing

| | Week 3 Dmpk | | Week 6 Dmpk | | Week 3 Dmpk | | Week 6 Dmpk | |
|---|---|---|---|---|---|---|---|---|
| | expression in | | expression in | | expression in | | expression in | |
| | REGN3892- | | REGN3892- | | REGN14572- | | REGN14572- | |
| | M3463-siRNA1 | | M3463-siRNA1 | | M3463-siRNA1 | | M3463-siRNA1 | |
| | relative to | | relative to | | relative to | | relative to | |
| | naked siRNA | | naked siRNA | | naked siRNA | | naked siRNA | |
| Tissue | (±SEM) | | (±SEM) | | (±SEM) | | (±SEM) | |
| Tibialis anterior | +5% | (±3%) | +6% | (±12%) | −44% | (±8%) | −51% | (±2%) |
| Diaphragm | −2% | (±7%) | +7% | (±7%) | −40% | (±5%) | −50% | (±4%) |
| Soleus | −3% | (±5%) | +1% | (±4%) | −53% | (±9%) | −43% | (±3%) |
| Quadricep | +48% | (±6%) | +2% | (±4%) | −47% | (±6%) | −60% | (±1%) |
| Gastrocnemius | +6% | (±3%) | +5% | (±3%) | −62% | (±3%) | −48% | (±4%) |
| Liver | +13% | (±4%) | −1% | (±3%) | +7% | (±6%) | −1% | (±9%) |
| Heart | −15% | (±4%) | −15% | (±2%) | −24% | (±4%) | −10% | (±5%) |
| Kidney | −11% | (±6%) | +7% | (±15%) | +5% | (±8%) | +7% | (±4%) |

### Example 12. Generation of antibody-steroid conjugates

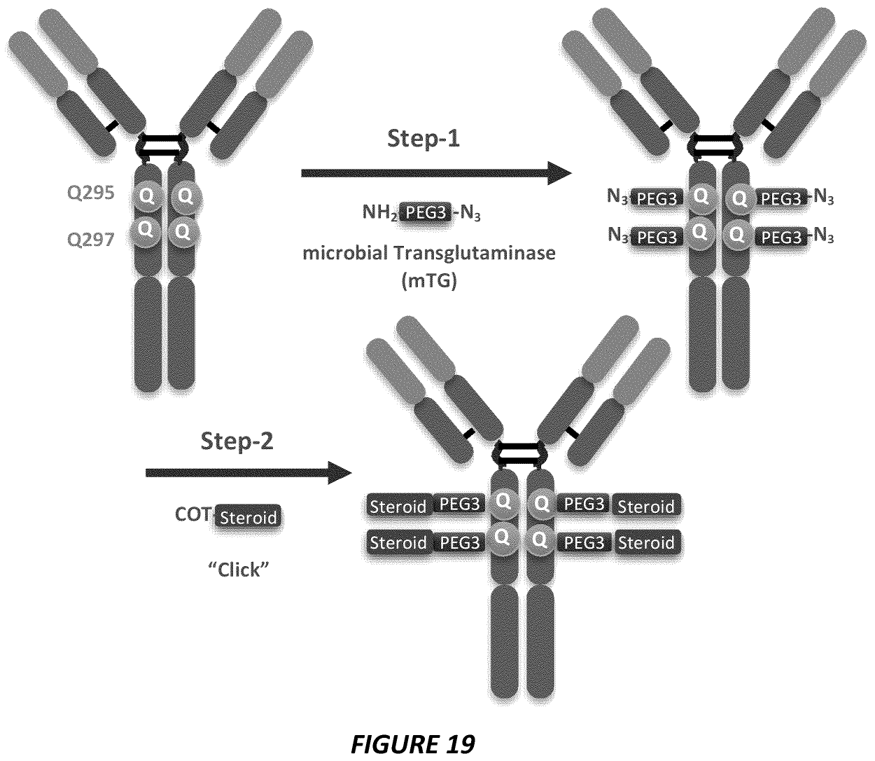

Anti-CACNG1 antibodies were conjugated with steroid linker payload (LP) via a two-step site-specific conjugation method, shown in FIG. 19. The first step was microbial transglutaminase (mTG)-mediated enzymatic attachment of a small linker (LK), azido-PEG3-amine, to Q295 and Q297 sites on heavy chain. Specifically, antibodies were mixed with 40-200 molar equivalents of azido-PEG3-amine; followed by mTG (0.5-5 units mTG per mg of antibody) resulting in a final concentration of the antibody at 1-25 mg/mL. The reaction mixture was incubated at 25-37° C. for 1-5 hours with gentle mixing. Excess linker and mTG were removed by size-exclusion chromatography (SEC) or Protein-A affinity chromatography (ProA). The second step employed the attachment of a cyclooctyne-spacer-payload to the azido-functionalized antibody via a [2+3] cycloaddition (aka copper-free click chemistry). Specifically, azido-functionalized antibodies were mixed with an organic co-solvent (e.g., DMA, DMSO) then slowly added 5-10 molar equivalents of LP. The reaction mixture was incubated at 30-37° C. for 10⁻⁴⁸ hours with gentle mixing. Steroid conjugates were purified by SEC or Tangential Flow Filtration (TFF), formulated in PBS with 5% glycerol or histidine/sucrose buffer, then stored at −80° C. The protein concentration was measured by a UV-vis spectrophotometer. Overall protein recovery 50-80%. ΔΔCt C monomer purity (>90%) was determined by analytical SEC, SDS-PAGE and hydrophobic interaction chromatography (HIC). ADCs were further characterized by liquid chromatography electrospray ionization mass spectrometry (LC-ESI-MS) to calculate the drug-to-antibody ratio (DAR>3.7).

### Procedure for Generating Azido-Functionalized Antibody (Step-1)

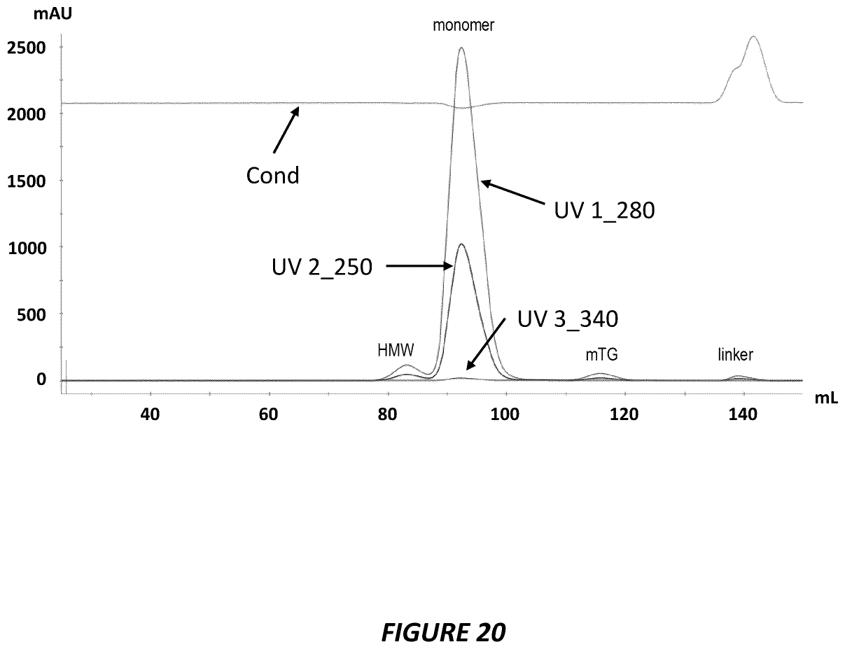

50 mg anti-CACNG1 antibody was mixed with 80 molar equivalents of azido-PEG3-amine; followed by microbial transglutaminase (1 unit mTG per mg of antibody) resulting in a final concentration of the antibody at 17 mg/mL. The reaction mixture was incubated at 37° C. for 2 hours with gentle mixing. Excess linker and mTG were removed by size-exclusion chromatography (SEC) (AKTA pure, Superdex 200 pg) (FIG. 20). The drug-to-antibody ratio (DAR=4.0) was characterized by liquid chromatography electrospray ionization mass spectrometry (LC-ESI-MS).

### Procedure for Site-Specific Coniuqation of Cyclooctyne-Spacer-Payload to the Azido-Functionalized Antibody Via a [2+3] Cycloaddition (Step-2)

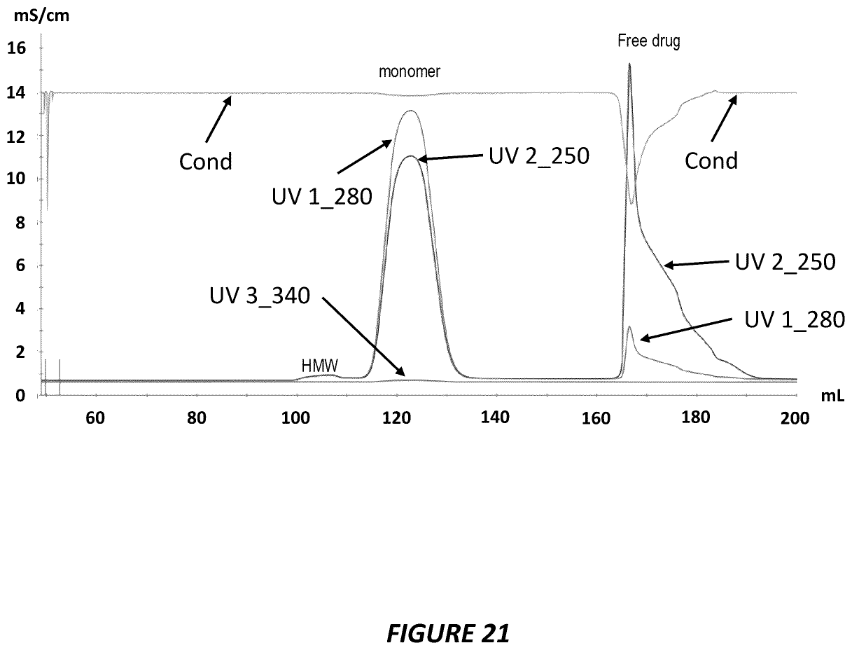

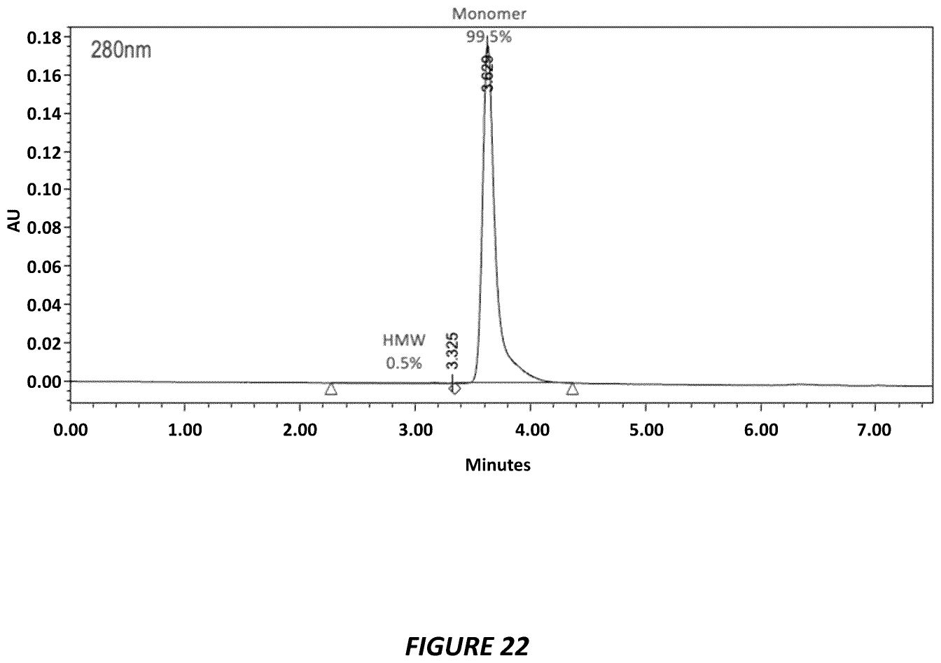

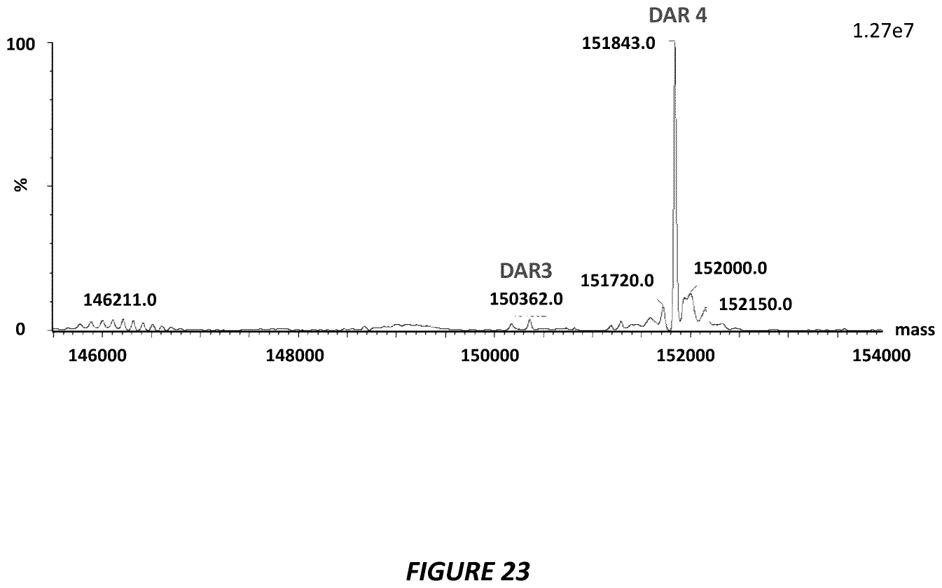

40.4 mg azido-functionalized anti-CACNG1 antibody was mixed with N,N-dimethylacetamine (DMA) then warmed to 37° C. 10 molar equivalents of steroid linker payload (LP) was slowly added. The reaction mixture was incubated at 37° C. overnight with gentle mixing. The reaction was monitored by ESI-MS. Upon the completion, crude ΔΔCt C was purified and concentrated by size-exclusion chromatography (SEC) (AKTA pure, Superdex 200 pg) (FIG. 21), formulated in PBS with 5% glycerol, then stored at −80° C. The protein concentration was measured by a UV-vis spectrophotometer. ΔΔCt C purity was determined by analytical SEC (FIG. 22). The ΔΔCt C was further characterized by liquid chromatography electrospray ionization mass spectrometry (LC-ESI-MS) to calculate the drug-to-antibody ratio (DAR=3.9) (FIG. 23).

### Methods for Characterizing Antibody-Steroid Conjugates

Analytical size exclusion chromatography (SEC) was performed to determine ΔΔCt C monomer purity. Sample was run on an ACQUITY Protein BEH SEC column (200A, 1.7 um, 4.6 mm×150 mm) installed on an ACQUITY UPLC instrument (Waters), using 10 mM phosphate, 1.0 M sodium perchlorate, 5% v/v isopropanol as mobile phase, at a flow rate of 0.3 mL/min, and monitored UV-vis absorbance at 280 nm using an ex PDA detector (Waters). The analytical SEC result (FIG. 22) indicated 96.5% monomer purity.

Liquid chromatography electrospray ionization mass spectrometry (LC-ESI-MS) analysis was performed to determine the drug distribution profile and to calculate the average drug-to-antibody ratio (DAR) (Table 9-1, FIG. 23). Each sample (20 μL at 1 mg/mL) was loaded onto an ACQUITY UPLC Protein BEH C4 column (10K psi, 300A, 1.7 um, 75 um×100 mm; mass spectrum was acquired on a Waters Synapt G2-Si mass spectrometer. As shown in FIG. 23, the major peak is DAR 4 species.

TABLE 9-1
Drug distribution profile and average drug-to-antibody
ratio average drug-to-antibody ratio (DAR)

| | | | Mass |
|---|---|---|---|
| | | DAR by | Species |
| Antibody | Modification | ESI-MS | (m/z) |
| Anti-CACNG1 Ab1 | None | N/A | 144910 |
| Anti-CACNG1 Ab1 | Azido-PEG3-amine | 4.0 | 145716 |
| Anti-CACNG1 Ab1 | Steroid LP | 3.9 | 151566 |
| Anti-CACNG1 Ab2 | None | N/A | 145188 |
| Anti-CACNG1 Ab2 | Azido-PEG3-amine | 4.0 | 145994 |
| Anti-CACNG1 Ab2 | Steroid LP | 3.9 | 151843 |

### Example 13. CACNG1 Antibody-Dihydrotestosterone (DHT) Conjugate Androgen Reporter Assay

CACNG1 is the γ1 subunit of the dihydropyridine receptor that is expressed specifically in skeletal muscle. Therefore, antibodies generated against CACNG1 could be used to deliver conjugated therapeutic payloads specifically to skeletal muscle to enhance therapeutic efficacy in muscle and reduce off-target toxicity. For example, conjugation of the potent metabolite of testosterone, dihydrotestosterone (DHT), to CACNG1 antibodies, may allow for androgen receptor signaling in muscle, leading to increased muscle mass and function. Here, CACNG1 antibodies conjugated to a linker with DHT payload were tested in an androgen receptor (AR) reporter cell line to determine whether these antibody conjugates can specifically activate the AR in CACNG1-expressing cells in vitro.

To assess signaling via the AR, LNCaP cells were transfected with lentivirus (Qiagen; ARE.Luc Cignal Lenti) to generate a stable cell line that expressed AR-luciferase reporter (AR.Luc). A subset of these selected cells was transduced to express human CACNG1 and further selected, with this cell line being referred to as hCACNG1.AR.Luc.

For the bioassay, AR.Luc or hCACNG1.AR.Luc cells were plated at 5,000 cells/well in OptiMEM and 0.5% charcoal-stripped FBS in PDL-coated 96-well plates. Cells were then incubated for 24, 48, or 27 hours with CACNG1 antibodies or isotype control antibody conjugated to DHT (via M3004 linker-payload), or unconjugated DHT alone (M608). All antibodies were conjugated with DHT at a drug-antibody-ratio (DAR) of ˜4. After the respective timepoints, cells were lysed and incubated with One-GLO buffer, and luminescence was read on an Envision plate reader. Relative luminescence units (RLU) were plotted against log concentration in mol/L, adjusted for DAR.

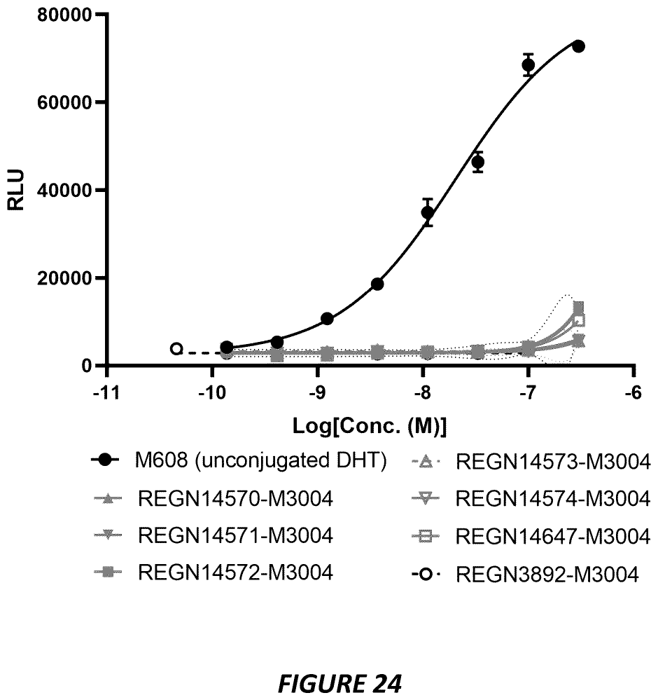

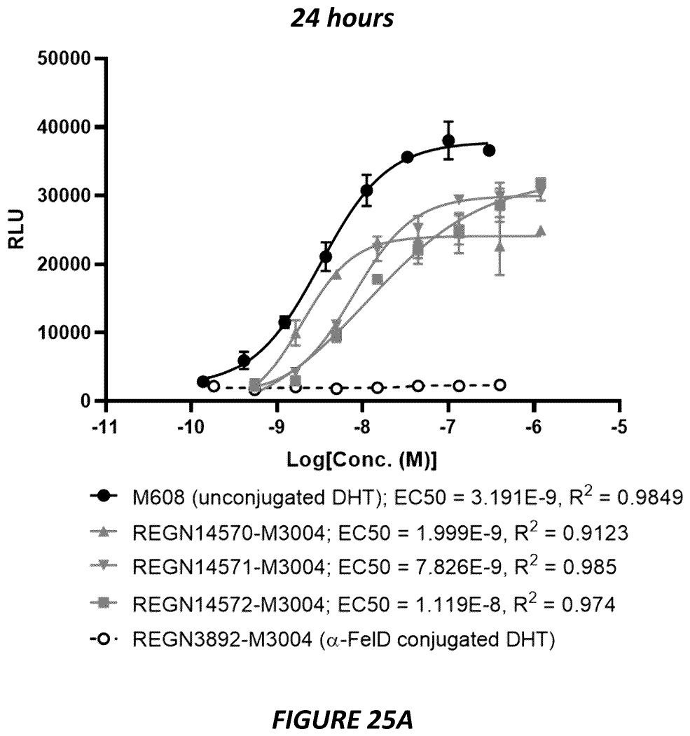

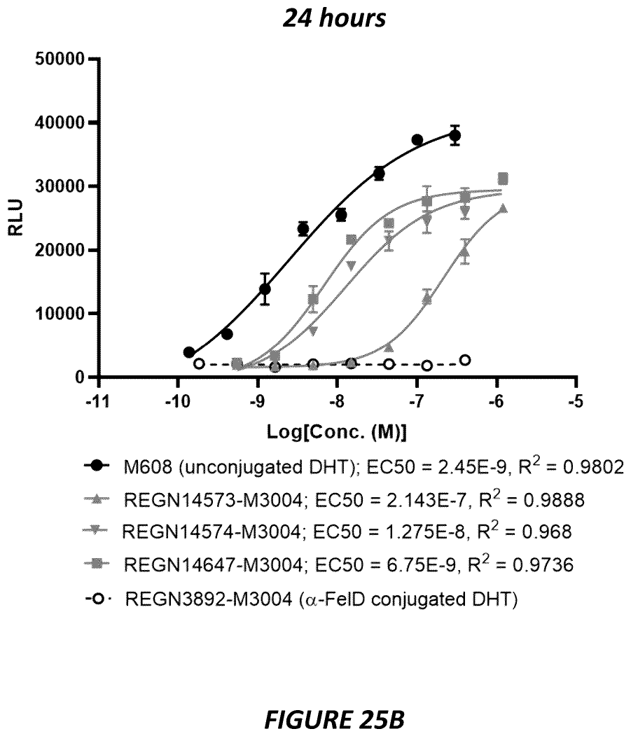

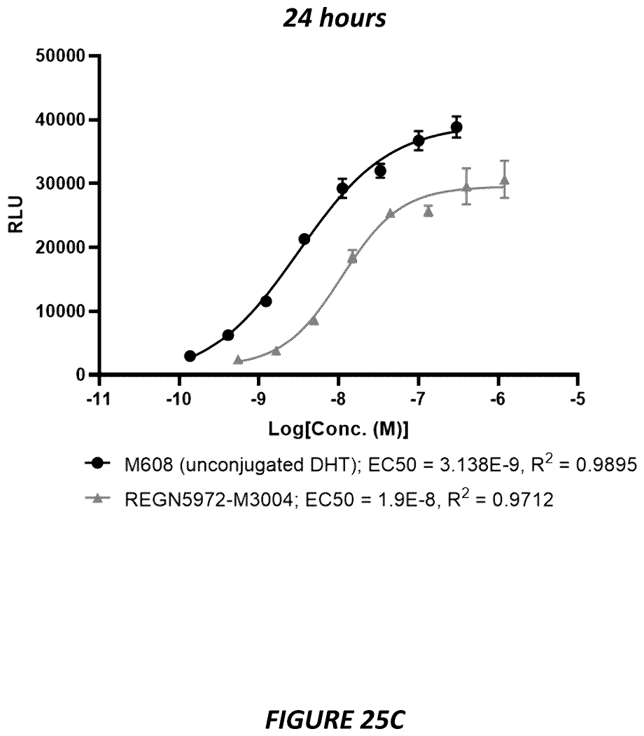

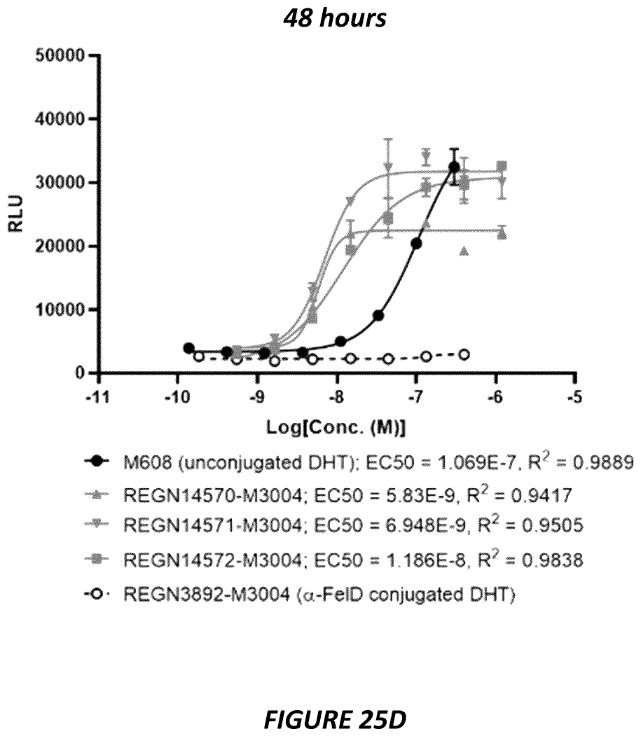

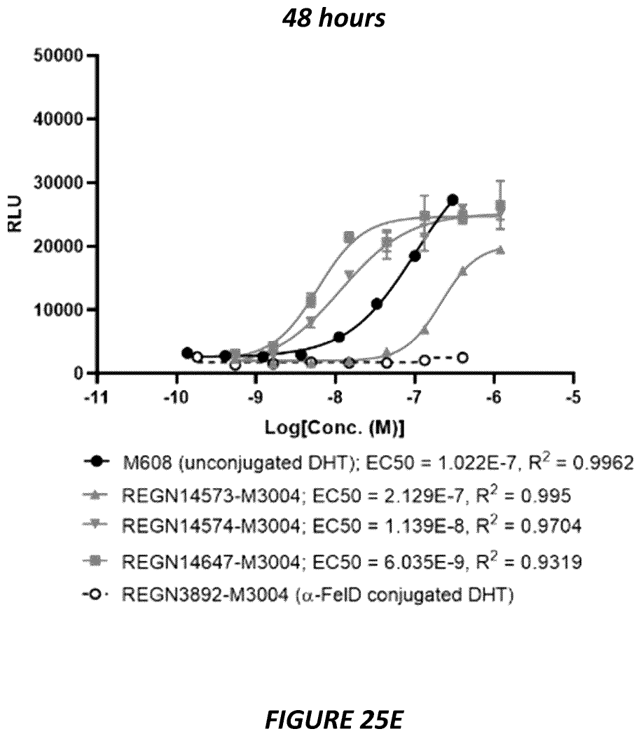

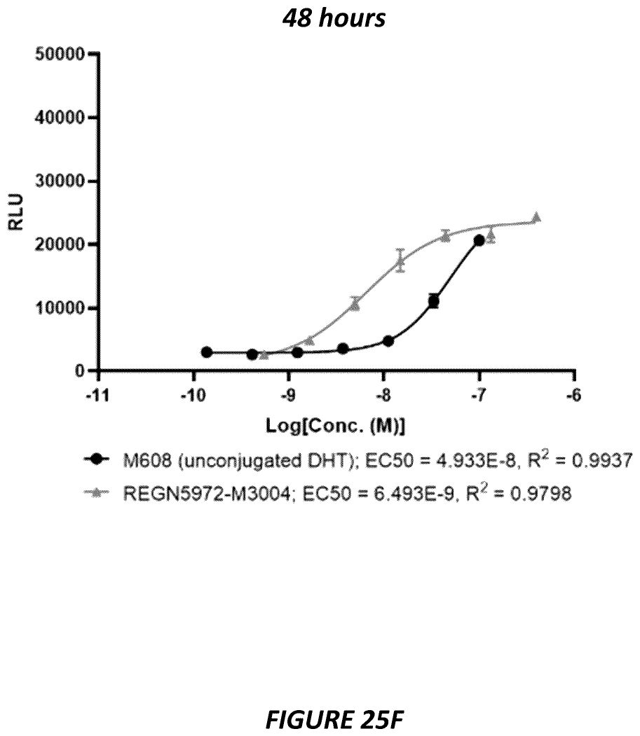

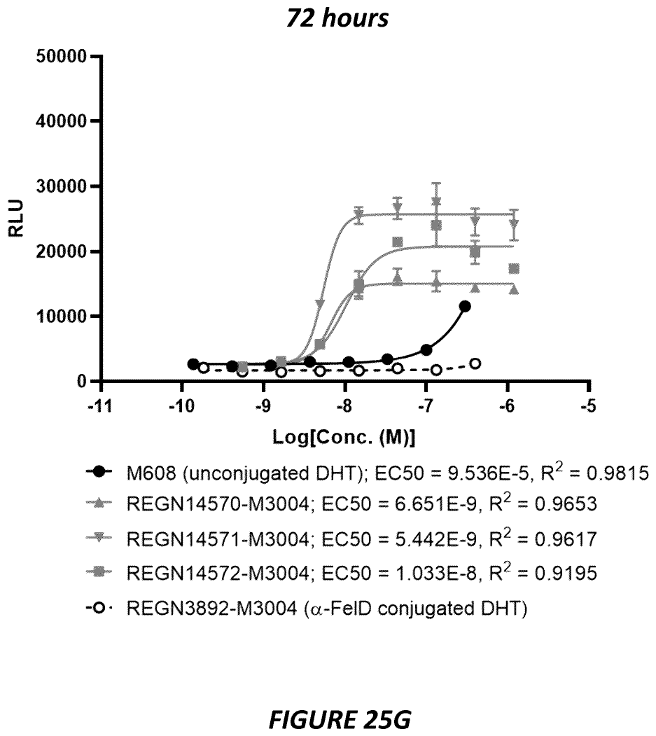

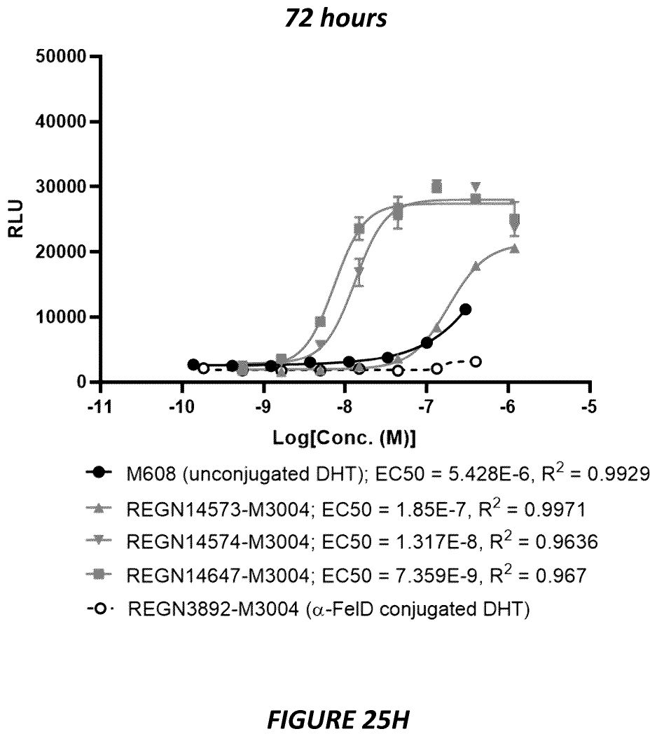

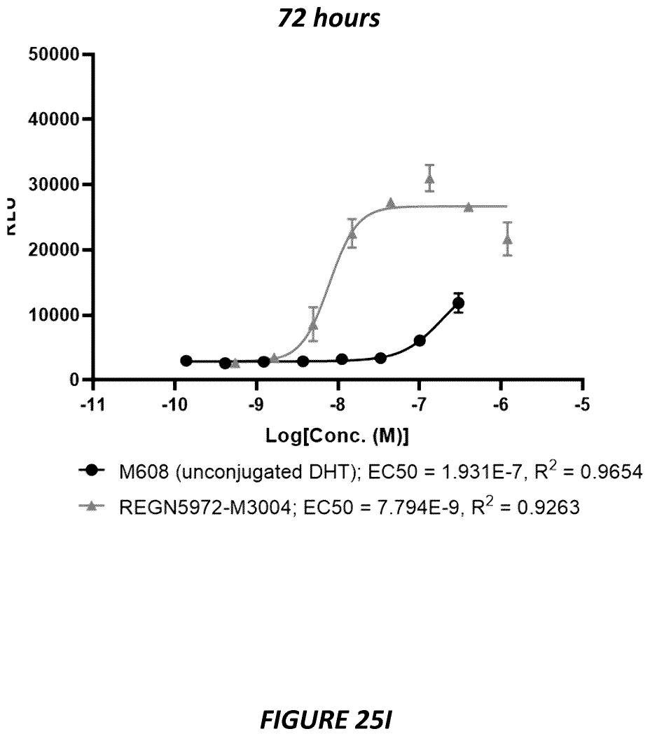

Unconjugated DHT (M608) activated AR in both AR.Luc (FIG. 24) and hCACNG1.AR.Luc cell lines (FIGS. 25A-251), whereas DHT conjugated to an isotype control antibody (REGN3892-M3004) did not activate AR in either of these cell lines. Thus, only unconjugated DHT was shown to activate androgen receptor in this assay, while none of the CACNG1 antibodies conjugated to DHT showed any appreciable activation of the androgen receptor in this cell line that does not express hCACNG1. Several CACNG1 antibody-DHT conjugates activated AR only in the hCACNG1.AR.Luc cell line (FIGS. 25A-251), but not in the AR.Luc cell line (FIG. 24). While the efficacy and potency of AR activation of CACNG1 antibody-DHT conjugates was lower than that of unconjugated DHT at 24 hours following treatment, activation of the AR was sustained at 48 and 72 hours with these conjugates, whereas unconjugated AR signal decreased substantially at these timepoints. Overall, these data demonstrate that conjugation of DHT to CACNG1 antibodies allows for specific activation of the AR in cells expressing hCACNG1, and that DHT conjugated to CACNG1 antibodies maintains sustained AR signaling over several days in hCACNG1-expressing cells in vitro.

### Example 14. Budesonide Delivery to Skeletal Muscle Via CACNG1 Targeted ΔΔCt C

C2C12 cells (ATCC, catalog #CRL-1772) were seeded in collagen I coated tissue culture (TC) treated 24-well dishes (Corning, catalog #356408) at 50,000 cells/well in growth media consisting of DMEM (Gibco, catalog #11965092)+10% FBS (Gibco, catalog #A3160402)+Pen/Strep (Gibco, catalog #15140148). After 24 hours, cells were differentiated using DMEM+2% horse serum (Gibco, catalog #16050130). After 3 days of differentiation, with media changes every other day, media was changed to phenol-free DMEM (Gibco, catalog #A1443001)+GlutaMAX (Gibco, catalog #35050079)+2% charcoal stripped horse serum (Valley Biomedical, catalog #AS3053CS). C2C12 myotubes were treated with either 100 nM budesonide with linker (M3123), or an equivalent volume of DMSO, or 25.6 nM of REGN3892-M404-M3123 (anti-FeID conjugated with budesonide; DAR 3.9), 24.4 nM REGN14570-M404-M3123 (anti-CACNG1 conjugated with budesonide; DAR 4.1) or 25.6 nM of REGN14572-M404-M3123 (anti-CACNG1 conjugated with budesonide; DAR 3.9). Myotubes were treated for 48 hours. For the 24-hour treatment, myotubes were treated with the same concentrations of reagents after 4 days of differentiation for 24 hours.

On completion of treatment, media was removed from cells and 1 ml of Trizol reagent (Invitrogen, catalog #15596026) was added per well, before samples were frozen at −80° C. Total RNA was isolated using MagMAX™-96 for Microarrays Total RNA Isolation Kit (Ambion by Life Technologies, catalog #AM1839) according to the manufacturer's instructions. Isolated RNA was treated with DNAse I (Thermo Scientific, catalog #EN0525) and reverse transcribed using the SuperScript VILO cDNA synthesis Master Mix (Invitrogen, catalog #11754050). Synthesized cDNA was diluted 10-fold in nuclease free water and then assayed by qPCR for the following genes using the listed TaqMan assays: Klf15 (Mm00517792_m1), Fkbp5 (Mm00487406_m1), Pdk4 (Mm01166879_m1), Ppib (Mm00478295_m1) and Actb (Mm00607939_s1). Reactions were prepared using the TaqMan Gene Expression Master Mix, loaded onto MicroAmp 384-well plate (ThermoFisher Scientific, catalog #4309849) and run on the QuantStudio 6 Flex instrument with the standard TaqMan protocol. Results were analyzed by the ΔΔCt method, and data were analyzed using 2-way ANOVA with Dunnett's multiple comparisons test comparing treated groups to untreated, with p<0.05 significance.

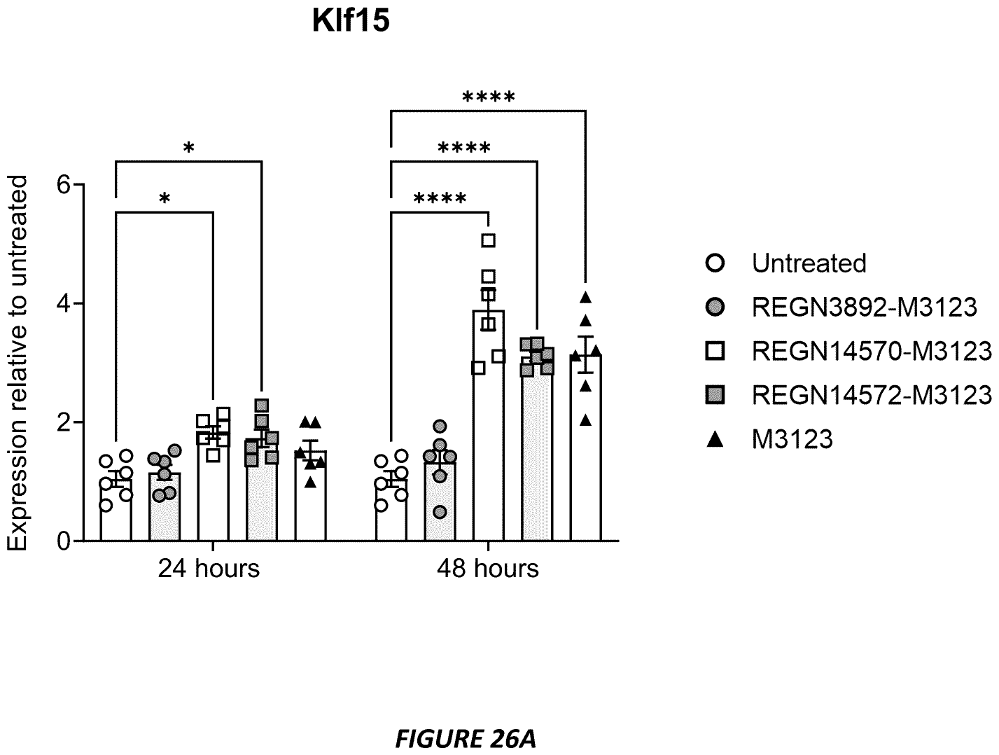

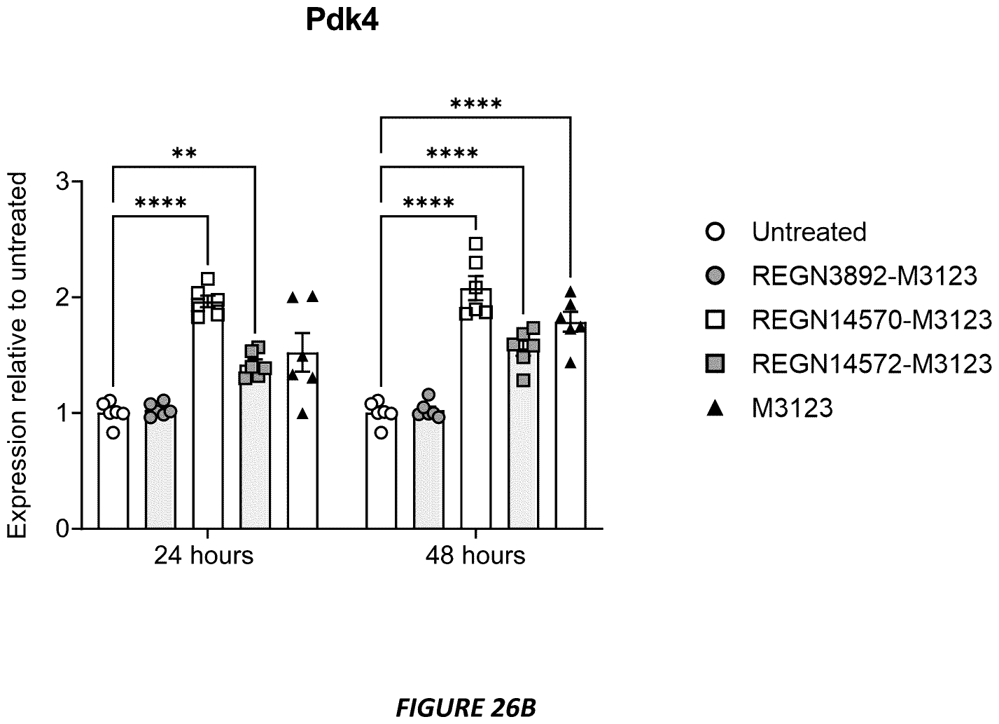

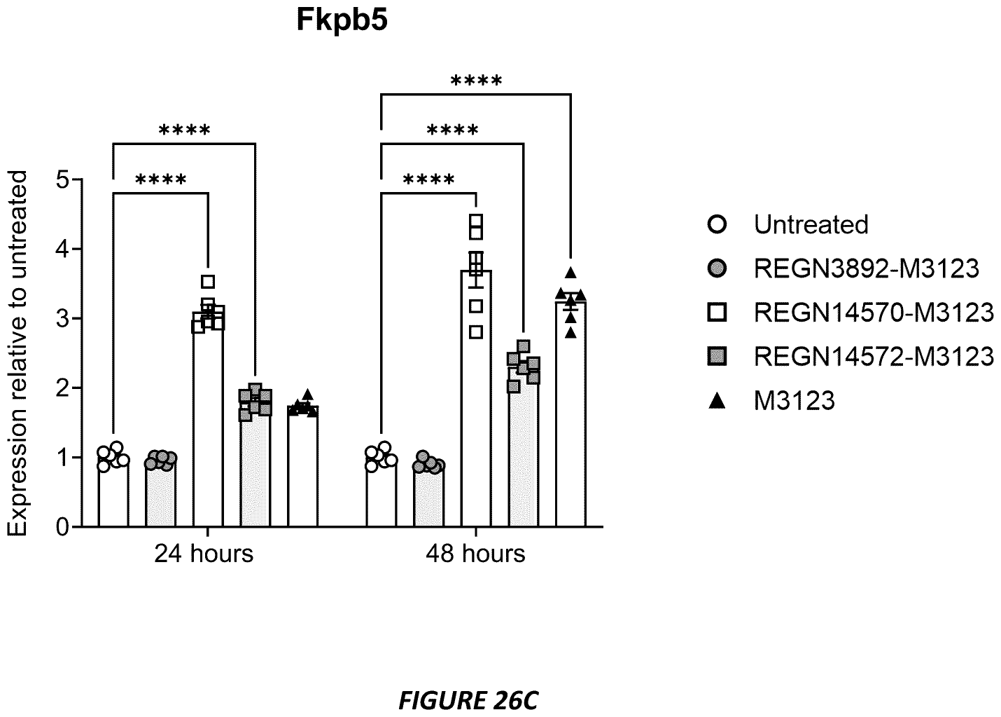

Glucocorticoid responsive genes were induced in C2C12 myotubes following treatment with either M3123 or CACNG1-targeted ADCs compared to untreated myotubes. FIGS. 26A-26C depict significant increases (*p<0.05, **p<0.01, ***p<0.001, ****p<0.0001) in C2C12 myotube expression of Klf15, Pdk4, and Fkbp5, following 24 or 48 hours of treatment with either M3123, REGN14570-M3123, or REGN14572-M3123. Isotype control Ab (REGN3892) conjugated to M3123 did not induce increased gene expression of any of these targets at any timepoint.

### Example 15. Biosensor Imaging in Primary Human and Mouse Myotubes

This Example relates to evaluation of the ability of the anti-CACNG1 antibodies described herein to localize to endosomal/lysosomal intracellular compartments. Table 10⁻¹ shows a description of the anti-CACNG1 antibodies tested in the present Example.

TABLE 10-1
Anti-CACNG1 antibodies tested

| Target | REGN # | Lot # | Modification | Isotype |
|---|---|---|---|---|
| CACNG1 | REGN14570 | REGN14570- | M378- | hIgG1 |
| | | L5 | Biosensor | N297Q |
| CACNG1 | REGN14572 | REGN14572- | M378- | hIgG1 |
| | | L11 | Biosensor | N297Q |
| hIgG1Q | REGN3892 | REGN3892- | M378- | hIgG1N297Q |
| isotype | | L80 | Biosensor | |
| control | | | | |

To determine the ability of anti-CACNG1 antibodies to localize to endosomal/lysosomal intracellular compartments, a cathepsin B cleavable biosensor probe was conjugated to REGN14570 and REGN14572 anti-CACNG1 antibodies and to an isotype control antibody REGN3892-hlgG1Q. The biosensor is an enzyme-activatable dual fluorescent molecule with a Cathepsin B selective peptide sequence separating Alexa Fluor 568 (AF568) from Alexa Fluor 647 (AF647). The intact biosensor emits only AF647 signal, whereas the peptide cleaved biosensor emits both AF568 and AF647 signals. As such, the AF568 signal is used as a marker for internalization to the late endosome/lysosome compartments.

To assess biosensor conjugate activity in cells, primary human skeletal myoblasts (Cook Myosite Cat #SkMDC SK-1111) and C2C12 mouse myoblasts (ATCC Cat #CRL-1772) were utilized. Human skeletal myoblasts (HuSKM) were maintained in MyoTonic Basal Media (Cook Myosite Cat #MB-2222) supplemented with MyoTonic Growth Supplement (Cook Myosite Cat #MS-3333) and grown in a 37° C. incubator with 5% CO₂. HuSKM were seeded in PhenoPlate 96-well, black, optically clear flat-bottom, collagen-coated plates (Perkin Elmer #6055700) at 20,000 cells/well and grown for 24 hours. The growth media was changed to MyoTonic Differentiation Media (Cook Myosite Cat #MD-5555) and replaced every 2 days. C2C12 mouse myoblasts were maintained in DMEM with 10% FBS and 1% penicillin-streptomycin supplement and grown in a 37° C. incubator with 5% CO₂. C2C12 cells were seeded in PhenoPlate 96-well, black, optically clear flat-bottom, collagen-coated plates (Perkin Elmer #6055700) at 10,000 cells/well. After 24 hours, growth media was replaced with differentiation media (DMEM with 2% horse serum) and placed in a 37° C. incubator with 7.5% CO₂. After 6 days (HuSKM myotubes) or 3 days (C2C12 myotubes) of differentiation, whole cells were labeled with CFSE Cell trace (Molecular Probe, Cat #C34554), incubated with the biosensor conjugates (6.7 nM) for 4 hours or 24 hours, imaged on the Opera Phenix (Perkinelmer), and AF568 fluorescence intensity per CFSE labeled cell area was determined via Harmony (PerkinElmer) software.

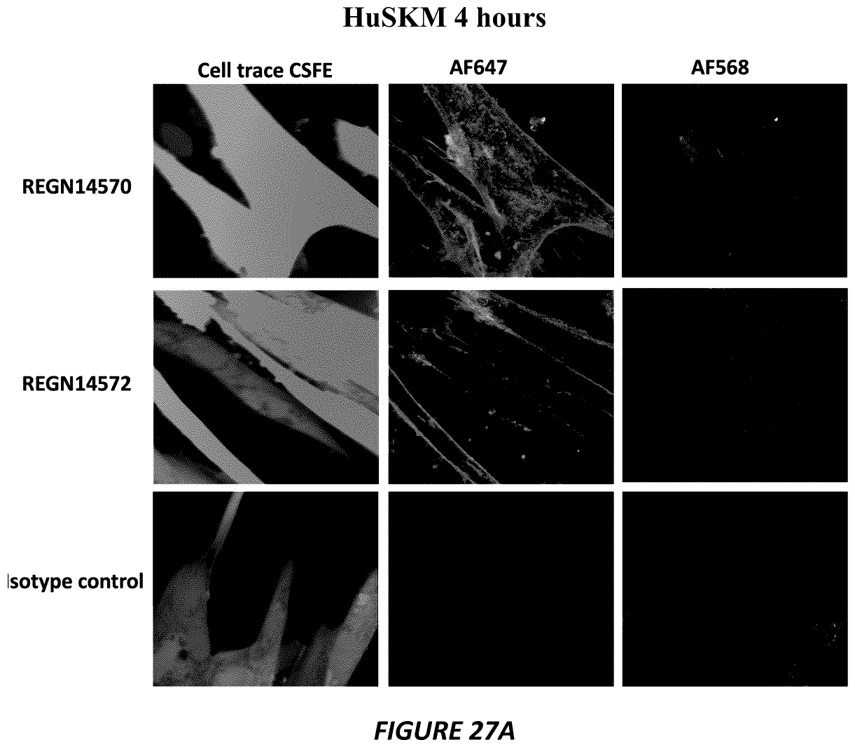

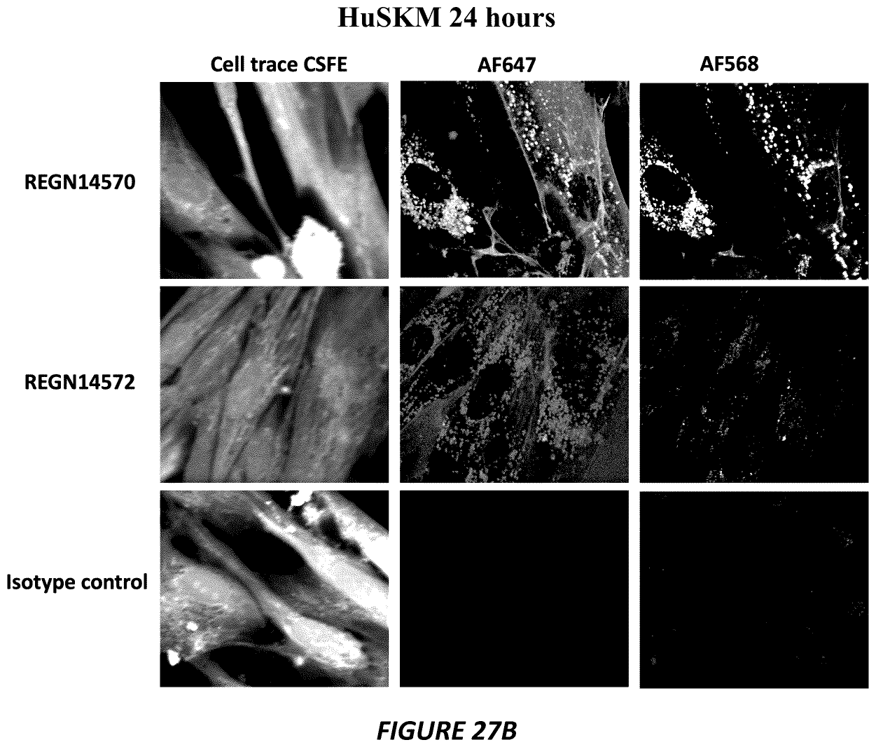

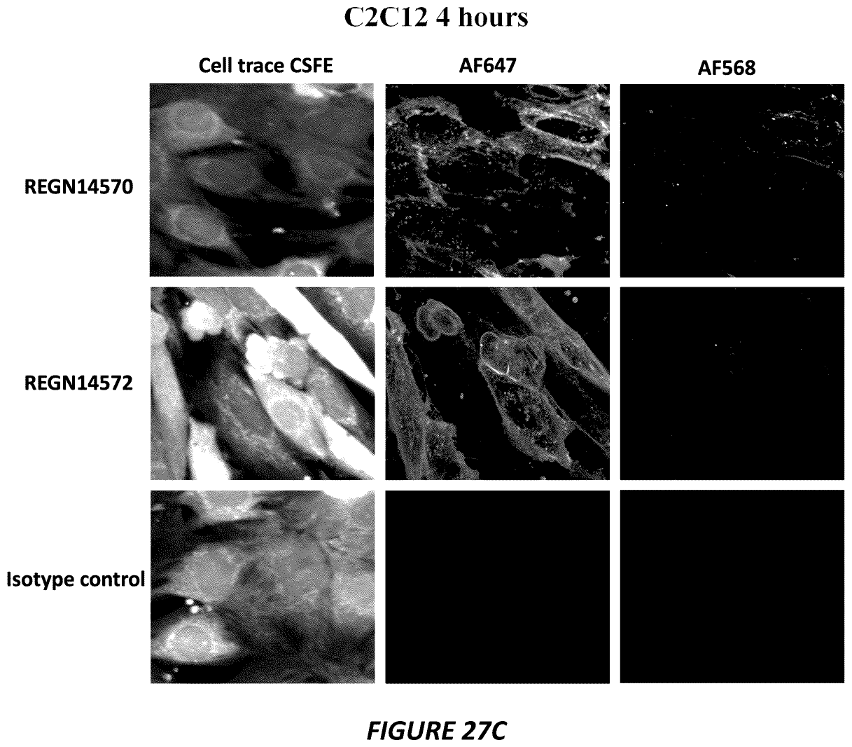

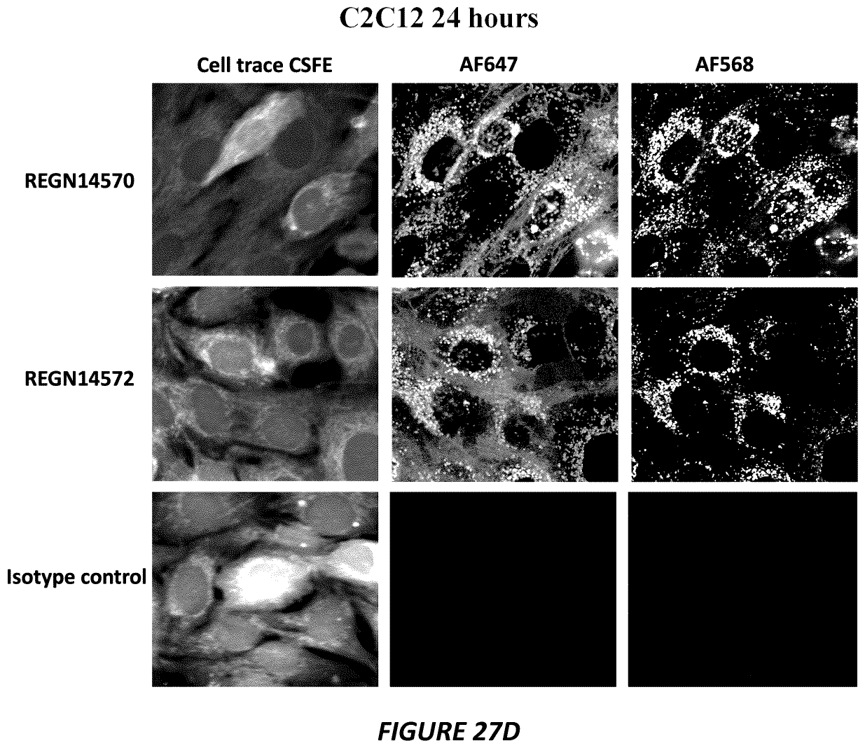

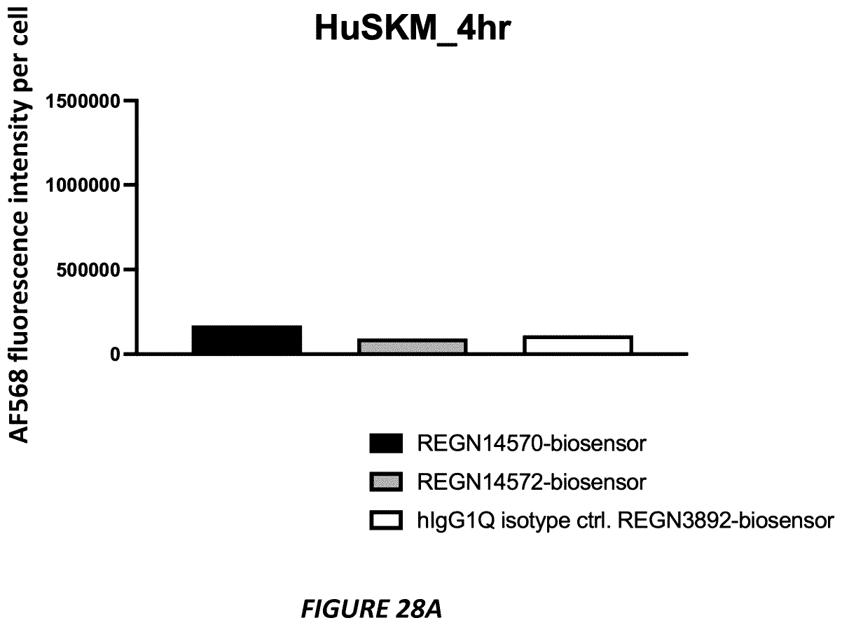

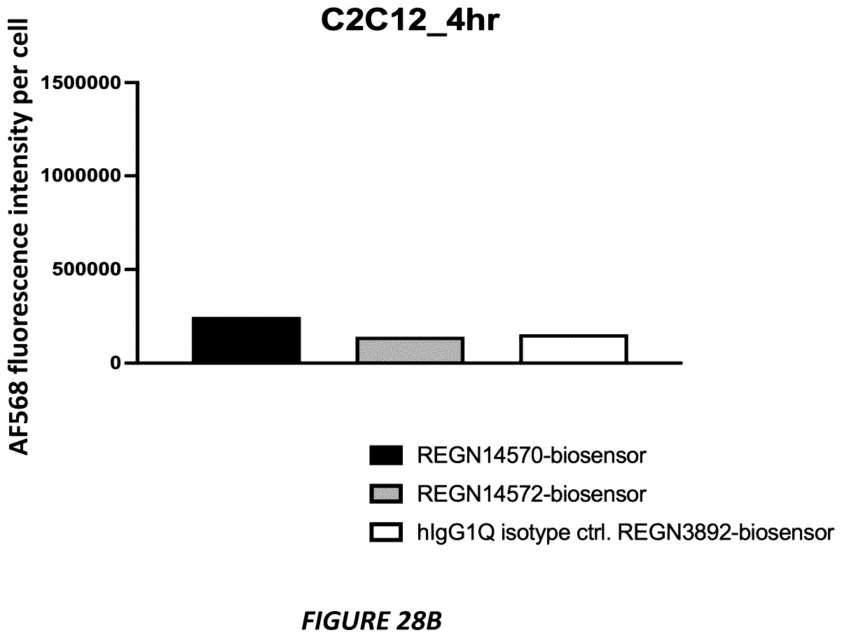

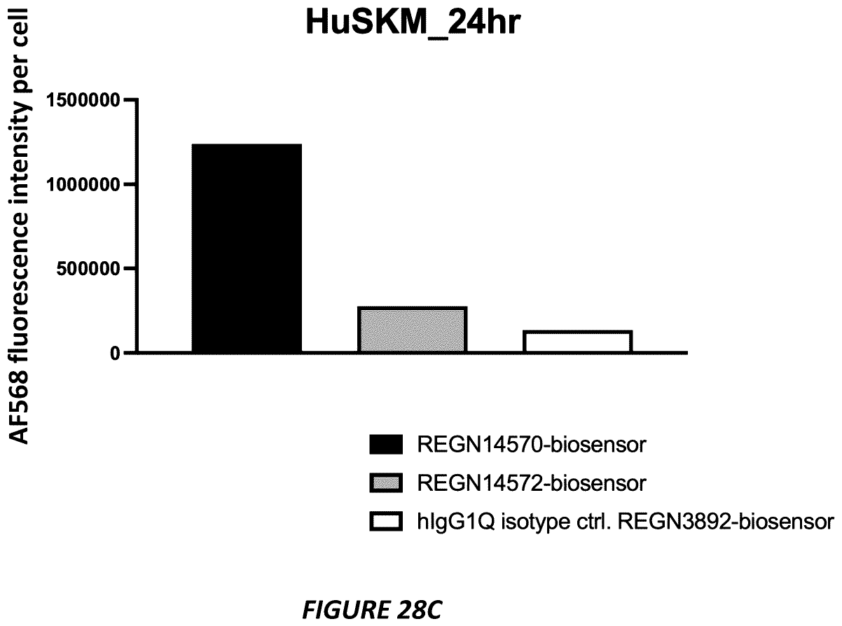

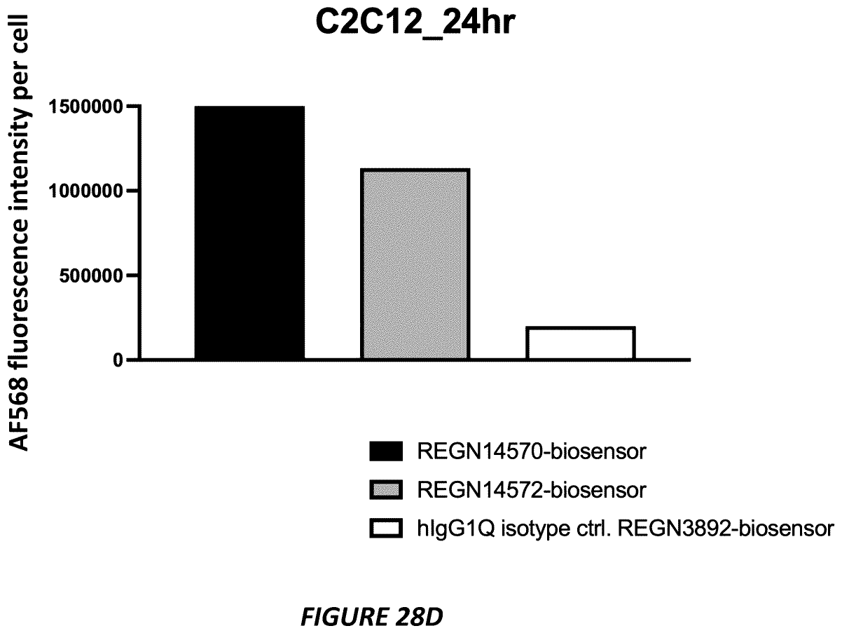

Representative images are shown in FIGS. 27A-27D and the relative AF568 fluorescence intensity per cell for each biosensor conjugate is shown in FIGS. 28A-28D. As shown in FIG. 27A and FIG. 27C, total binding to differentiated HuSKM myotubes and C2C12 mouse myotubes was observed from the AF647 signal for both REGN14570 and REGN14572 biosensor conjugates at 4 hours. In contrast, no AF568 signal from the cleaved biosensor was observed at 4 hours suggesting that the anti-CACNG1 biosensor conjugates had not yet reached the late endosomal/lysosomal compartments. As shown in FIG. 27B and FIG. 27D, both AF647 and AF568 signals were observed at 24 hours for the REGN14570 and REGN14572 biosensor conjugates in differentiated HuSKM myotubes and C2C12 mouse myotubes suggesting internalization into the late endosomal/lysosomal compartments in human and mouse muscle cells. No detectable binding or internalization of the control antibody was observed.

The AF568 fluorescence intensity per cell was calculated and is shown in FIGS. 28A-28D. Consistent with the images in FIGS. 27A-27D, the cleaved biosensor signal (AF568) was low at 4 hours and barely detectable above control monoclonal antibody (mAb) levels. In contrast, cleaved biosensor AF568 was detectable at 24 hours in both C2C12 and HUSKM cells at levels above control mAb further supporting that anti-CACNG1 mAbs have the capacity to traffic into the late endosomal/lysosomal intracellular compartment in differentiated HuSKM myotubes and C2C12 mouse myotubes.

### Example 16. High Content Imaging-Based Cell Binding Assay in Primary Myotubes and In Vitro Secondary Cell Killing Assay in Primary Myotubes and Engineered Cell Lines

To test the ability of anti-CACNG1 antibodies described herein to bind, internalize, and deliver payloads, cell binding and indirect cell killing assays were developed with primary human skeletal myoblasts (Cook Myosite Cat #SkMDC SK-1111), C2C12 mouse myoblasts (ATCC Cat #CRL-1772), and HEK293 cells engineered to express human and mouse CACNG1 (HEK293/hCACNG1 and HEK293/mCACNG1, respectively). To generate the HEK293/hCACNG1 and HEK293/mCACNG1 cell lines, FuGEGE 6 (Promega, Cat #E2691/5/3) was used to transfect HEK293HZ cells with a human CACNG1 (hCACNG1; amino acid M1-H222, Accession #NP_000718.1) or mouse CACNG1 (mCACNG1; amino acid M1-H223, Accession #NP_031608.1) expression vector. HEK293HZ is a highly transfectable subclone of HEK293 cells (1) authenticated via STIR (short tandem repeat) profiling to be 81% identical to HEK293 cells sourced from ATCC). The HEK293HZ cell line was maintained in DMEM+10% FBS+1% L-glutamine/penicillin/streptomycin, while HEK293/hCACNG1 and HEK293/mCACNG1 cell lines were maintained in DMEM+10% FBS+1% L-glutamine/penicillin/streptomycin+500 ug/mL G418. Human skeletal myoblasts (HuSKM) were maintained in MyoTonic Basal Media (Cook Myosite Cat #MB-2222) supplemented with MyoTonic Growth Supplement (Cook Myosite Cat #MS-3333) and grown in a 37° C. incubator with 5% CO₂. HuSKM were seeded in collagen-coated 96-well plates (Greiner Bio-One #655956) at 8,500 cells/well and grown for 24 hours. The growth media was changed to MyoTonic Differentiation Media (Cook Myosite Cat #MD-5555) and replaced every 2 days. Myotubes were assayed after 6 days of differentiation. C2C12 mouse myoblasts were maintained in DMEM with 10% FBS and 1% penicillin-streptomycin supplement and grown in a 37° C. incubator with 5% CO₂. C2C12 cells were seeded in collagen-coated 96-well plates (Greiner Bio-One #655956) at 10,000 cells/well for the cell binding assay and 5,000 cells/well for the cell killing assay. After 24 hours, growth media was replaced with differentiation media (DMEM with 2% horse serum) and placed in a 37° C. incubator with 7.5% CO₂. C2C12 myotubes were assayed after 3 days of differentiation. HEK293/hCACNG1, HEK293/mCACNG1, and HEK293 cells were seeded at 10,000 cells (cell binding assay) or 2,000 (cell killing assay) per well in 96-well black collagen coated plates (Greiner Bio-One, cat #655956) in complete growth media and grown overnight at 37° C. in 5% CO₂.

Relative binding of anti-CACNG1 antibodies was assessed across the entire panel of cell lines via imaging analysis. Briefly, cells were labeled with CarboxyFluoroscein Succinimidyl Ester (CFSE) Cell trace (Molecular Probe, Cat #C34554) to identify the whole cell region, and then incubated with anti-CACNG1 antibodies and isotype control antibodies at final concentrations ranging from 100 nM to 15.2 μM for 30 minutes on ice in PBS+2% FBS+0.2% NaN₃ (antibody dilution buffer). Following a wash with antibody dilution buffer, cells were incubated with 10 μg/mL of Alexa fluor 647 affinipure F(ab′)₂ fragment goat anti-human IgG, Fcy fragment specific (Jackson, Cat #109-606-170) or Alexa fluor 647 affinipure F(ab′)₂ fragment goat anti-mouse IgG, Fcy fragment specific (Jackson, Cat #115-606-071) for 30 minutes on ice. After one wash with antibody dilution buffer, samples were fixed in CytoFix (BD Biosciences, Cat #554655) diluted 1:1 in PBS and supplemented with a 1:2000 dilution of Hoechst 33342 (Thermo, Cat #H1399) for 20 minutes on ice. Images were acquired with a 10× objective on the Opera Phenix (Perkin Elmer) and relative fluorescence units (RFU) was determined using Harmony software (Perkin Elmer). Imaging binding was expressed as EC₅₀ and fold RFU binding above isotype control antibody levels, and results are summarized in Tables 11-1, 11-2, and 11-4.

As shown in Table 11-1, anti-CACNG1 antibodies bind to HEK293/hCACNG1 cells with EC₅₀ values ranging from of 0.21 nM to >100 nM and signal to noise values ranging from 8.3 to 246.6, and to primary HuSKM with EC₅₀ values ranging from 0.13 nM to >100 nM and signal to noise values ranging from 105.3 to 1060. A subset of these antibodies bind HEK293/mCACNG1 with EC₅₀ values ranging from 0.26 nM to >100 nM and signal to noise values ranging from 5.3 to 69.4, and on C2C12 mouse myoblasts with EC₅₀ values ranging from 2.3 nM to >100 nM and signal to noise values ranging from 4.9 to 8.4 folds. In a separate experiment (Table 11-2), anti-CACNG1 REGN9909 binds to HEK293/hCACNG1 cells with an EC₅₀ value of 1.6 nM and a signal to noise value of 5.4, to primary HuSKM with an EC₅₀ value of 14 nM and a signal to noise value of 171.1, and to C2C12 mouse myoblasts with an EC₅₀ value of 8.3 nM and a signal to noise value of 4.2. Anti-CACNG1 REGN10712 binds to primary HuSKM with an EC₅₀ value 53 μM and a signal to noise value of 102.0, and to C2C12 mouse myoblasts with an EC₅₀ value of >100 nM and a signal to noise value of 8.6. In another separate experiment, in Table 11-4, anti-CACNG1 REGN9908 binds to primary HuSKM with an EC₅₀ value if 14.8 nM and a signal to noise value of 163.9.

All anti-CACNG1 antibodies weakly bound to non-CACNG1 expressing HEK293 cells with signal to noise <2.8. Control mAbs in Tables 11-1, Table 11-2 and Table 11-4 bound weakly on all tested cells with EC₅₀ values >100 nM and signal to noise <140.8.

TABLE 11-1
anti-CACNG1 antibodies binding by imaging in primary human skeletal myotubes
(HuSKM), and C2C12 mouse myotubes, HEK293/hCACNG1, HEK293/mCACNG1, and HEK293

| | | | | | 293/ | | 293/ | | | |
|---|---|---|---|---|---|---|---|---|---|---|
| | HuSKM | | C2C12 | | hCACNG1 | | mCACNG1 | | HEK293 Hz | |
| Test | EC₅₀ | | EC₅₀ | | EC₅₀ | | EC₅₀ | | EC₅₀ | |
| article | (M) | S/N | (M) | S/N | (M) | S/N | (M) | S/N | (M) | S/N |
| REGN10713 | NB | 105.3 | NB | 2.0 | NB | 3.1 | >1.0E−07 | 2.7 | NB | 1.8 |
| REGN10715 | 2.0E−10 | 1060.0 | 2.3E−09 | 8.4 | 2.7E−09 | 91.3 | 2.6E−10 | 69.4 | NB | 2.3 |
| REGN10716 | 1.3E−08 | 465.6 | >1.0E−07 | 6.3 | >1.0E−07 | 35.9 | >1.0E−07 | 21.4 | NB | 2.2 |
| REGN10717 | 6.9E−08 | 292.3 | >1.0E−07 | 4.9 | >1.0E−07 | 77.4 | >1.0E−07 | 23.6 | NB | 2.8 |
| REGN10783 | 2.9E−09 | 734.0 | NB | 1.1 | 8.5E−09 | 229.8 | NB | 1.7 | NB | 1.4 |
| REGN7660 | NB | 128.1 | NB | 1.0 | >1.0E−07 | 8.3 | >1.0E−07 | 7.2 | NB | 1.7 |
| H4H31265P2 | 3.3E−09 | 249.8 | NB | 0.8 | 1.1E−08 | 246.6 | NB | 1.2 | NB | 1.1 |
| REGN10728 | 1.3E−09 | 466.8 | NB | 1.8 | 2.4E−09 | 138.5 | NB | 0.8 | NB | 0.9 |
| REGN10729 | 3.4E−09 | 572.3 | NB | 2.4 | 7.3E−09 | 155.4 | NB | 1.9 | NB | 1.1 |
| REGN5972 | 2.9E−09 | 313.3 | NB | 1.6 | 6.6E−09 | 172.5 | >1.0E−07 | 5.3 | NB | 2.8 |
| H2aM31929N | 1.9E−09 | 251.7 | NB | 1.2 | 6.2E−09 | 84.0 | NB | 1.0 | NB | 1.3 |
| H2aM31944N | 4.8E−10 | 320.8 | NB | 0.7 | 3.3E−09 | 93.4 | >1.0E−07 | 2.2 | NB | 2.7 |
| H1M31941N | 1.3E−10 | 559.1 | NB | 1.2 | 2.1E−10 | 51.2 | NB | 1.4 | NE | 1.4 |
| REGN653, | >1.0E−07 | <140.8 | NB | <2.3 | NB | <3.1 | NB | <1.4 | NE | <1.5 |
| REGN1097, | | | | | | | | | | |
| REGN3892, | | | | | | | | | | |
| REGN6387, | | | | | | | | | | |
| REGN1945 | | | | | | | | | | |

>=EC₅₀ values could not be determined with accuracy because the binding did not reach saturation within the tested antibody concentration range, and EC₅₀ is reported as greater than the highest tested concentration. NB = No binding detected within the tested antibody concentration range (S/N similar to control mAb); Control mAbs: REGN653, REGN1097, REGN3892, REGN6387, REGN1945.

TABLE 11-2
Anti-CACNG1 REGN9909 antibody binding by imaging
in primary human skeletal myotubes (HuSKM), and
C2C12 mouse myotubes, HEK293/hCACNG1 and HEK293

| | HuSKM | | C2C12 | | 293/hCACNG1 | | HEK293 Hz | |
|---|---|---|---|---|---|---|---|---|
| Test | EC₅₀ | | EC₅₀ | | EC₅₀ | | EC₅₀ | |
| article | (M) | S/N | (M) | S/N | (M) | S/N | (M) | S/N |
| REGN10712 | 5.3E−11 | 102.0 | >1.0E−07 | 8.6 | NT | NT | NT | NT |
| REGN9909 | 1.4E−08 | 171.1 | 8.3E−09 | 4.2 | 1.6E−09 | 5.4 | NB | 0.6 |
| REGN10716 | 1.2E−08 | 81.1 | >1.0E−07 | 5.2 | >1.0E−07 | 2.2 | NB | 2.2 |
| REGN1097 | >1.0E−07 | 1.4 | >1.0E−07 | 1 | >1.0E−07 | 0.9 | NB | 1.4 |

>=EC₅₀ values could not be determined with accuracy because the binding did not reach saturation within the tested antibody concentration range, and EC₅₀ is reported as greater than the highest tested concentration. NB = No binding detected within the tested antibody concentration range (S/N similar to control mAb); NT = not tested; Control mAb: REGN1097.

An indirect cell killing assay was used to assess the ability of anti-CACNG1 antibodies to internalize and deliver payloads into cells. In vitro cytotoxicity of the anti-CACNG1 antibodies alone or in combination with Fab anti-human Fc or Fab anti-mouse Fc secondary antibodies conjugated to Duocarmycin DM (Moradec, Cat #AH-202-DD-50 or Cat #AM-202-DD-50) were evaluated using the CellTiter-Glo 2.0 Assay Kit (Promega, Cat #G9243), in which the quantity of ATP present is used to determine the number of viable cells in culture. A free payload control, Duocarmycin, was also included. For the assay, three-fold serial dilutions of anti-CACNG1 antibodies or isotype controls were prepared in dilution media (Optimem+0.1% BSA) and added to primary HuSKM or mouse C2C12 myoblasts at final concentrations ranging from 10 nM to 0.0256 μM, and to engineered human (HEK293/hCACNG1) and mouse (HEK293/mCACNG1) cell lines at final concentrations ranging from 1 nM to 0.00256 μM. The Duocarmycin DM secondary antibody conjugates were added at a constant concentration of 30 nM, and the cells were incubated for 6 days at 37° C., 5% CO₂. Three-fold serial dilutions of free Duocarmycin control payloads were prepared in 100% DMSO, transferred to fresh dilution media, and then added to the cells at a final constant DMSO concentration of 0.2% and final payload concentrations ranging from 100 nM to 15.2 μM. The last well in each dilution series (untreated wells) served as blank controls containing only media (anti-CACNG1 antibodies) or media plus 0.2% DMSO (payloads) and was plotted as a continuation of the 3-fold serial dilution. CellTiter Glo 2.0 (100 ml) was added to each well, plates were mixed for 2 minutes on an orbital shaker, and plates were incubated at room temperature for 10 minutes. Relative light units (RLUs) were measured on an Envision luminometer (PerkinElmer) and cell viability was expressed as a percentage of the untreated (100% viable) cells. IC₅₀ values were determined using a four-parameter logistic equation over a 10-point dose response curve (GraphPad Prism). The maximum % kill was also determined for each test article as follows: 100—minimum percent viability. The IC₅₀ value and maximum % kill of each test article is shown in Table 11-3 and 11-4.

As shown in Table 11-3, anti-CACNG1 antibodies co-incubated with anti-Fc-duocarmycin killed HEK293/hCACNG1 cells with IC₅₀ values ranging from of 0.356 μM to 132 pM and maximum % kill values ranging from 88.5% to 97.0%. A subset of these antibodies also killed HEK293/mCACNG1 cells when co-incubated with anti-Fc toxin conjugated antibodies with IC₅₀ values ranging from 0.187 μM to >1.0 nM and maximum % kill values ranging from 2.4% to 96.1%. Anti-CACNG1 antibodies in the indirect killing assay also killed primary HuSKM cells with IC₅₀ values ranging from of 9.7 μM to >10 nM and maximum % kill values ranging from 0% to 99.6%. A subset of these antibodies also killed C2C12 mouse myoblasts when con-incubated with anti-Fc toxin conjugated antibodies with IC₅₀ values ranging from 3.56 μM to >10 nM and maximum % kill values ranging from 0% to 99.9%. All tested anti-CACNG1 antibodies in combination anti-Fc-toxin conjugated antibodies were weakly cytotoxic in CACNG1 negative HEK293 cells with IC₅₀ values >1 nM. Non-binding control mAbs with anti-Fc-toxin co-incubation and all anti-CACNG1 antibodies alone (data not shown) were weakly cytotoxic in all tested cells with IC₅₀ values >1 nM. The free payload Duocarmycin killed all tested cells with IC₅₀ values ranging from 1.58 μM to 3.23 nM. In a separate experiment, in Table 11-4, anti-CACNG1 REGN9908 co-incubated with anti-Fc-duocarmycin killed primary HuSKM cells with IC₅₀ value of 8.59 μM and maximum % kill value of 98.8%.

TABLE 11-3
Indirect cell killing of anti-CACNG1 antibodies in HEK293/hCACNG1, HEK293/mCACNG1,
HEK293, primary human skeletal myotubes (HuSKM), and mouse myotubes (C2C12)

| | HEK293/ | | HEK293/ | | | | | | | |
|---|---|---|---|---|---|---|---|---|---|---|
| | hCACNG1 | | mCACNG1 | | HEK293 | | HuSKM | | C2C12 | |
| | EC₅₀/ | | EC₅₀/ | | EC₅₀/ | | EC₅₀/ | | EC₅₀/ | |
| Test | IC₅₀ | % | IC₅₀ | % | IC₅₀ | % | IC₅₀ | % | IC₅₀ | % |
| article | (M) | KILL | (M) | KILL | (M) | KILL | (M) | KILL | (M) | KILL |
| REGN10713 | 1.32E−10 | 88.5 | >1E−09 | 2.4 | >1E−09 | 0.0 | >1.0E−08 | 0.0 | >1.0E−08 | 0 |
| REGN10715 | 6.29E−13 | 96.4 | 1.87E−13 | 96.1 | >1E−09 | 64.3 | 9.70E−12 | 99.6 | 4.69E−12 | 99.8 |
| REGN10716 | 2.95E−12 | 95.1 | 8.93E−12 | 91.2 | >1E−09 | 0.0 | 3.40E−10 | 61.7 | 1.67E−11 | 99.9 |
| REGN10717 | 8.06E−13 | 97.0 | 1.18E−12 | 96.0 | >1E−09 | 44.2 | 3.64E−11 | 99.6 | 3.56E−12 | 99.8 |
| REGN10783 | 1.78E−12 | 95.4 | >1E−09 | 23.7 | >1E−09 | 1.0 | 2.16E−10 | 99.3 | >1.0E−08 | 0 |
| REGN7660 | 1.83E−12 | 95.6 | 1.34E−11 | 92.3 | >1E−09 | 2.3 | 2.20E−10 | 99.0 | 2.37E−11 | 99.8 |
| H4H31265P2 | 2.45E−12 | 94.6 | >1E−09 | 22.9 | >1E−09 | 1.8 | 5.60E−10 | 95.6 | >1.0E−08 | 3.4 |
| REGN10728 | 5.19E−13 | 95.8 | >1E−09 | 5.1 | >1E−09 | 1.4 | 3.81E−11 | 99.3 | >1.0E−08 | 1.6 |
| REGN10729 | 1.12E−12 | 95.8 | >1E−09 | 9.3 | >1E−09 | 0.6 | 1.56E−10 | 99.3 | >1.0E−08 | 0 |
| REGN5972 | 1.39E−12 | 95.4 | >1E−09 | 11.0 | >1E−09 | 0.5 | 5.85E−10 | 97.4 | >1.0E−08 | 0 |
| REGN9909 | 2.35E−12 | 95.2 | 6.12E−11 | 88.5 | >1E−09 | 0.3 | 1.83E−11 | 99.5 | NT | NT |
| H2aM31929N | 2.77E−12 | 95.0 | >1E−09 | 18.3 | >1E−09 | 0.0 | 3.10E−11 | 96.3 | >1.0E−08 | 7.5 |
| H2aM31944N | 3.56E−13 | 95.7 | >1E−09 | 4.3 | >1E−09 | 1.3 | 1.16E−11 | 98.6 | >1.0E−08 | 0 |
| H1M31941N | 2.17E−12 | 95.4 | >1E−09 | 4.9 | >1E−09 | 0.0 | 1.12E−10 | 91.2 | >1.0E−08 | 0.4 |
| REGN653, | >1E−09 | <4.0 | >1E−09 | <17.0 | >1E−09 | <7.0 | >1.0E−08 | 0.0 | >1.0E−08 | <29.4 |
| REGN1097, | | | | | | | | | | |
| REGN3892, | | | | | | | | | | |
| REGN6387, | | | | | | | | | | |
| REGN1945 | | | | | | | | | | |
| Duocarmycin | 8.98E−12 | 99.7 | 1.58E−12 | 99.8 | 3.96E−11 | 99.6 | 3.02E−10 | 97.3 | 3.23E−09 | 99.6 |

Control mAbs: REGN653, REGN1097, REGN3892, REGN6387, REGN1945;
Free payload: Duocarmycin

TABLE 11-4
Anti-CACNG1 REGN9908 antibody binding in primary human skeletal myotubes
(HuSKM), and indirect cell killing of anti-CACNG1 antibodies in primary
human skeletal myotubes (HuSKM), and mouse myotubes (C2C12)

| | Binding | | 2^{nd} Cell Killing | | | | | |
|---|---|---|---|---|---|---|---|---|
| | HuSKM | | HuSKM | | C2C12 | | HEK293 Hz | |
| | EC₅₀ | | EC₅₀/ | % | EC₅₀/ | % | EC₅₀/ | % |
| Test article | (M) | S/N | IC₅₀ (M) | KILL | IC₅₀ (M) | KILL | IC₅₀ (M) | KILL |
| REGN9908 | 1.48E−08 | 163.9 | 8.59E−11 | 98.8 | ND | 57.5 | ND | 8.9 |
| REGN9909 | 6.51E−09 | 141.4 | 9.68E−12 | 99.1 | ND | 9 | ND | 4.3 |
| REGN1097 | NB | 5.5 | ND | 0 | ND | 0 | ND | 0 |

NB = No binding detected within the tested antibody concentration range; ND = Not determined (while cell killing may have been detected, the cell killing curve was not saturated so no EC₅₀ could be determined); Control mAb: REGN1097.

TABLE 11-5
Summary of test articles of the present Example

| Test article | Isotype |
|---|---|
| REGN10712 | hIgG4 |
| REGN10713 | hIgG4 |
| REGN10715 | hIgG4 |
| REGN10716 | hIgG4 |
| REGN10717 | hIgG4 |
| REGN10783 | hIgG4 |
| REGN7660 | hIgG4 |
| H4H31265P2 | hIgG4 |
| REGN1945 | hIgG4 |
| REGN10728 | hIgG4us |
| REGN10729 | hIgG4us |
| REGN6387 | hIgG4us |
| REGN5972 | hIgG1 Q |
| REGN3892 | hIgG1 Q |
| REGN9908 | mIgG2b |
| REGN9909 | mIgG2a |
| H2aM31929N | mIgG2a |
| H2aM31944N | mIgG2a |
| REGN1097 | mIgG2a |
| H1M31941N | mIgG1 |
| REGN653 | mIgG1 |

### REFERENCE

Graham F., Simley J., Russell W. and Nairn R. (1977) Characteristics of a Human Cell Line Transformed by DNA from Human Adenovirus Type 5. Journal of General Virology, 36:1, 59-72)

All references cited herein are incorporated by reference to the same extent as if each individual publication, database entry (e.g., Genbank sequences or GenelD entries), patent application, or patent, was specifically and individually indicated to be incorporated by reference. This statement of incorporation by reference is intended by Applicants to relate to each and every individual publication, database entry (e.g., Genbank sequences or GenelD entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

We claim:

1. An antigen-binding protein that binds specifically Calcium Voltage-Gated Channel Auxiliary Subunit Gamma 1 (CACNG1), comprising:
(i) an HCVR that comprises the HCDR1, HCDR2, and HCDR3 of an HCVR comprising the amino acid sequence set forth in SEQ ID NO: 1, 9, 17, 25, 33, 41, 49, 57, 65, 73, 81, 89, 97, 105, 113, 121, 129, 137, 429, or 451 (or a variant thereof);
and/or
(ii) an LCVR that comprises the LCDR1, LCDR2, and LCDR3 of an LCVR comprising the amino acid sequence set forth in SEQ ID NO: 5, 13, 21, 29, 37, 45, 53, 61, 69, 77, 85, 93,101, 109, 117, 125, 133, 141, 437, or 459 (or a variant thereof).

2. The antigen-binding protein of claim 1 which comprises:
(1) an HCVR comprising the HCDR1, HCDR2, and HCDR3 of an HCVR that comprises the amino acid sequence set forth in SEQ ID NO: 1 (or a variant thereof);
and an LCVR comprising the LCDR1, LCDR2, and LCDR3 of an LCVR that comprises the amino acid sequence set forth in SEQ ID NO: 5 (or a variant thereof);
(2) an HCVR comprising the HCDR1, HCDR2, and HCDR3 of an HCVR that comprises the amino acid sequence set forth in SEQ ID NO: 9; and an LCVR comprising the LCDR1, LCDR2, and LCDR3 of an LCVR that comprises the amino acid sequence set forth in SEQ ID NO: 13 (or a variant thereof);
(3) an HCVR comprising the HCDR1, HCDR2, and HCDR3 of an HCVR that comprises the amino acid sequence set forth in SEQ ID NO: 17; and an LCVR comprising the LCDR1, LCDR2, and LCDR3 of an LCVR that comprises the amino acid sequence set forth in SEQ ID NO: 21 (or a variant thereof);
(4) an HCVR comprising the HCDR1, HCDR2, and HCDR3 of an HCVR that comprises the amino acid sequence set forth in SEQ ID NO: 25 (or a variant thereof); and an LCVR comprising the LCDR1, LCDR2, and LCDR3 of an LCVR that comprises the amino acid sequence set forth in SEQ ID NO: 29 (or a variant thereof);
(5) an HCVR comprising the HCDR1, HCDR2, and HCDR3 of an HCVR that comprises the amino acid sequence set forth in SEQ ID NO: 33 (or a variant thereof); and an LCVR comprising the LCDR1, LCDR2, and LCDR3 of an LCVR that comprises the amino acid sequence set forth in SEQ ID NO: 37 (or a variant thereof);
(6) an HCVR comprising the HCDR1, HCDR2, and HCDR3 of an HCVR that comprises the amino acid sequence set forth in SEQ ID NO: 41 (or a variant thereof); and an LCVR comprising the LCDR1, LCDR2, and LCDR3 of an LCVR that comprises the amino acid sequence set forth in SEQ ID NO: 45 (or a variant thereof);
(7) an HCVR comprising the HCDR1, HCDR2, and HCDR3 of an HCVR that comprises the amino acid sequence set forth in SEQ ID NO: 49 (or a variant thereof); and an LCVR comprising the LCDR1, LCDR2, and LCDR3 of an LCVR that comprises the amino acid sequence set forth in SEQ ID NO: 53 (or a variant thereof);
(8) an HCVR comprising the HCDR1, HCDR2, and HCDR3 of an HCVR that comprises the amino acid sequence set forth in SEQ ID NO: 57 (or a variant thereof); and an LCVR comprising the LCDR1, LCDR2, and LCDR3 of an LCVR that comprises the amino acid sequence set forth in SEQ ID NO: 61 (or a variant thereof);
(9) an HCVR comprising the HCDR1, HCDR2, and HCDR3 of an HCVR that comprises the amino acid sequence set forth in SEQ ID NO: 65 (or a variant thereof); and an LCVR comprising the LCDR1, LCDR2, and LCDR3 of an LCVR that comprises the amino acid sequence set forth in SEQ ID NO: 69 (or a variant thereof);
(10) an HCVR comprising the HCDR1, HCDR2, and HCDR3 of an HCVR that comprises the amino acid sequence set forth in SEQ ID NO: 73 (or a variant thereof); and an LCVR comprising the LCDR1, LCDR2, and LCDR3 of an LCVR that comprises the amino acid sequence set forth in SEQ ID NO: 77 (or a variant thereof);
(11) an HCVR comprising the HCDR1, HCDR2, and HCDR3 of an HCVR that comprises the amino acid sequence set forth in SEQ ID NO: 81 (or a variant thereof); and an LCVR comprising the LCDR1, LCDR2, and LCDR3 of an LCVR that comprises the amino acid sequence set forth in SEQ ID NO: 85 (or a variant thereof);
(12) an HCVR comprising the HCDR1, HCDR2, and HCDR3 of an HCVR that comprises the amino acid sequence set forth in SEQ ID NO: 89 (or a variant thereof); and an LCVR comprising the LCDR1, LCDR2, and LCDR3 of an LCVR that comprises the amino acid sequence set forth in SEQ ID NO: 93 (or a variant thereof);
(13) an HCVR comprising the HCDR1, HCDR2, and HCDR3 of an HCVR that comprises the amino acid sequence set forth in SEQ ID NO: 97 (or a variant thereof); and an LCVR comprising the LCDR1, LCDR2, and LCDR3 of an LCVR that comprises the amino acid sequence set forth in SEQ ID NO: 101 (or a variant thereof);
(14) an HCVR comprising the HCDR1, HCDR2, and HCDR3 of an HCVR that comprises the amino acid sequence set forth in SEQ ID NO: 105 (or a variant thereof); and an LCVR comprising the LCDR1, LCDR2, and LCDR3 of an LCVR that comprises the amino acid sequence set forth in SEQ ID NO: 109 (or a variant thereof);
(15) an HCVR comprising the HCDR1, HCDR2, and HCDR3 of an HCVR that comprises the amino acid sequence set forth in SEQ ID NO: 113 (or a variant thereof); and an LCVR comprising the LCDR1, LCDR2, and LCDR3 of an LCVR that comprises the amino acid sequence set forth in SEQ ID NO: 117 (or a variant thereof);
(16) an HCVR comprising the HCDR1, HCDR2, and HCDR3 of an HCVR that comprises the amino acid sequence set forth in SEQ ID NO: 121 (or a variant thereof); and an LCVR comprising the LCDR1, LCDR2, and LCDR3 of an LCVR that comprises the amino acid sequence set forth in SEQ ID NO: 125 (or a variant thereof);
(17) an HCVR comprising the HCDR1, HCDR2, and HCDR3 of an HCVR that comprises the amino acid sequence set forth in SEQ ID NO: 129 (or a variant thereof); and an LCVR comprising the LCDR1, LCDR2, and LCDR3 of an LCVR that comprises the amino acid sequence set forth in SEQ ID NO: 133 (or a variant thereof);
(18) an HCVR comprising the HCDR1, HCDR2, and HCDR3 of an HCVR that comprises the amino acid sequence set forth in SEQ ID NO: 137 (or a variant thereof); and an LCVR comprising the LCDR1, LCDR2, and LCDR3 of an LCVR that comprises the amino acid sequence set forth in SEQ ID NO: 141 (or a variant thereof);
(19) an HCVR comprising the HCDR1, HCDR2, and HCDR3 of an HCVR that comprises the amino acid sequence set forth in SEQ ID NO: 429 (or a variant thereof); and an LCVR comprising the LCDR1, LCDR2, and LCDR3 of an LCVR that comprises the amino acid sequence set forth in SEQ ID NO: 437 (or a variant thereof); or
(20) an HCVR comprising the HCDR1, HCDR2, and HCDR3 of an HCVR that comprises the amino acid sequence set forth in SEQ ID NO: 451 (or a variant thereof); and an LCVR comprising the LCDR1, LCDR2, and LCDR3 of an LCVR that comprises the amino acid sequence set forth in SEQ ID NO: 459 (or a variant thereof).

3. The antigen-binding protein of claim 1 or 2 which comprises:
(a) an HCVR that comprises: an HCDR1 comprising the amino acid sequence set forth in SEQ ID NO: 2 (or a variant thereof), an HCDR2 comprising the amino acid sequence set forth in SEQ ID NO: 3 (or a variant thereof), and an HCDR3 comprising the amino acid sequence set forth in SEQ ID NO: 4 (or a variant thereof); and
an LCVR that comprises: an LCDR1 comprising the amino acid sequence set forth in SEQ ID NO: 6 (or a variant thereof), an LCDR2 comprising the amino acid sequence set forth in SEQ ID NO: 7 (or a variant thereof), and an LCDR3 comprising the amino acid sequence set forth in SEQ ID NO: 8 (or a variant thereof);
(b) an HCVR that comprises: an HCDR1 comprising the amino acid sequence set forth in SEQ ID NO: 10 (or a variant thereof), an HCDR2 comprising the amino acid sequence set forth in SEQ ID NO: 11 (or a variant thereof), and an HCDR3 comprising the amino acid sequence set forth in SEQ ID NO: 12 (or a variant thereof); and
an LCVR that comprises: an LCDR1 comprising the amino acid sequence set forth in SEQ ID NO: 14 (or a variant thereof), an LCDR2 comprising the amino acid sequence set forth in SEQ ID NO: 15 (or a variant thereof), and an LCDR3 comprising the amino acid sequence set forth in SEQ ID NO: 16 (or a variant thereof);
(c) an HCVR that comprises: an HCDR1 comprising the amino acid sequence set forth in SEQ ID NO: 18 (or a variant thereof), an HCDR2 comprising the amino acid sequence set forth in SEQ ID NO: 19 (or a variant thereof), and an HCDR3 comprising the amino acid sequence set forth in SEQ ID NO: 20 (or a variant thereof); and
an LCVR that comprises: an LCDR1 comprising the amino acid sequence set forth in SEQ ID NO: 22 (or a variant thereof), an LCDR2 comprising the amino acid sequence set forth in SEQ ID NO: 23 (or a variant thereof), and an LCDR3 comprising the amino acid sequence set forth in SEQ ID NO: 24 (or a variant thereof);
(d) an HCVR that comprises: an HCDR1 comprising the amino acid sequence set forth in SEQ ID NO: 26 (or a variant thereof), an HCDR2 comprising the amino acid sequence set forth in SEQ ID NO: 27 (or a variant thereof), and an HCDR3 comprising the amino acid sequence set forth in SEQ ID NO: 28 (or a variant thereof); and
an LCVR that comprises: an LCDR1 comprising the amino acid sequence set forth in SEQ ID NO: 30 (or a variant thereof), an LCDR2 comprising the amino acid sequence set forth in SEQ ID NO: 31 (or a variant thereof), and an LCDR3 comprising the amino acid sequence set forth in SEQ ID NO: 32 (or a variant thereof);
(e) an HCVR that comprises: an HCDR1 comprising the amino acid sequence set forth in SEQ ID NO: 34 (or a variant thereof), an HCDR2 comprising the amino acid sequence set forth in SEQ ID NO: 35 (or a variant thereof), and an HCDR3 comprising the amino acid sequence set forth in SEQ ID NO: 36 (or a variant thereof); and
an LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof);
(f) an HCVR that comprises: an HCDR1 comprising the amino acid sequence set forth in SEQ ID NO: 42 (or a variant thereof), an HCDR2 comprising the amino acid sequence set forth in SEQ ID NO: 43 (or a variant thereof), and an HCDR3 comprising the amino acid sequence set forth in SEQ ID NO: 44 (or a variant thereof); and
an LCVR that comprises: an LCDR1 comprising the amino acid sequence set forth in SEQ ID NO: 46 (or a variant thereof), an LCDR2 comprising the amino acid sequence set forth in SEQ ID NO: 47 (or a variant thereof), and an LCDR3 comprising the amino acid sequence set forth in SEQ ID NO: 48 (or a variant thereof);
(g) an HCVR that comprises: an HCDR1 comprising the amino acid sequence set forth in SEQ ID NO: 50 (or a variant thereof), an HCDR2 comprising the amino acid sequence set forth in SEQ ID NO: 51 (or a variant thereof), and an HCDR3 comprising the amino acid sequence set forth in SEQ ID NO: 52 (or a variant thereof); and
an LCVR that comprises: an LCDR1 comprising the amino acid sequence set forth in SEQ ID NO: 54 (or a variant thereof), an LCDR2 comprising the amino acid sequence set forth in SEQ ID NO: 55 (or a variant thereof), and an LCDR3 comprising the amino acid sequence set forth in SEQ ID NO: 56 (or a variant thereof);
(h) an HCVR that comprises: an HCDR1 comprising the amino acid sequence set forth in SEQ ID NO: 58 (or a variant thereof), an HCDR2 comprising the amino acid sequence set forth in SEQ ID NO: 59 (or a variant thereof), and an HCDR3 comprising the amino acid sequence set forth in SEQ ID NO: 60 (or a variant thereof); and
an LCVR that comprises: an LCDR1 comprising the amino acid sequence set forth in SEQ ID NO: 62 (or a variant thereof), an LCDR2 comprising the amino acid sequence set forth in SEQ ID NO: 63 (or a variant thereof), and an LCDR3 comprising the amino acid sequence set forth in SEQ ID NO: 64 (or a variant thereof);
(i) an HCVR that comprises: an HCDR1 comprising the amino acid sequence set forth in SEQ ID NO: 66 (or a variant thereof), an HCDR2 comprising the amino acid sequence set forth in SEQ ID NO: 67 (or a variant thereof), and an HCDR3 comprising the amino acid sequence set forth in SEQ ID NO: 68 (or a variant thereof); and
an LCVR that comprises: an LCDR1 comprising the amino acid sequence set forth in SEQ ID NO: 70 (or a variant thereof), an LCDR2 comprising the amino acid sequence set forth in SEQ ID NO: 71 (or a variant thereof), and an LCDR3 comprising the amino acid sequence set forth in SEQ ID NO: 72 (or a variant thereof);
(j) an HCVR that comprises: an HCDR1 comprising the amino acid sequence set forth in SEQ ID NO: 74 (or a variant thereof), an HCDR2 comprising the amino acid sequence set forth in SEQ ID NO: 75 (or a variant thereof), and an HCDR3 comprising the amino acid sequence set forth in SEQ ID NO: 76 (or a variant thereof); and
an LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof);
(k) an HCVR that comprises: an HCDR1 comprising the amino acid sequence set forth in SEQ ID NO: 82 (or a variant thereof), an HCDR2 comprising the amino acid sequence set forth in SEQ ID NO: 83 (or a variant thereof), and an HCDR3 comprising the amino acid sequence set forth in SEQ ID NO: 84 (or a variant thereof); and
an LCVR that comprises: an LCDR1 comprising the amino acid sequence set forth in SEQ ID NO: 86 (or a variant thereof), an LCDR2 comprising the amino acid sequence set forth in SEQ ID NO: 87 (or a variant thereof), and an LCDR3 comprising the amino acid sequence set forth in SEQ ID NO: 88 (or a variant thereof);
(l) an HCVR that comprises: an HCDR1 comprising the amino acid sequence set forth in SEQ ID NO: 90 (or a variant thereof), an HCDR2 comprising the amino acid sequence set forth in SEQ ID NO: 91 (or a variant thereof), and an HCDR3 comprising the amino acid sequence set forth in SEQ ID NO: 92 (or a variant thereof); and
an LCVR that comprises: an LCDR1 comprising the amino acid sequence set forth in SEQ ID NO: 94 (or a variant thereof), an LCDR2 comprising the amino acid sequence set forth in SEQ ID NO: 95 (or a variant thereof), and an LCDR3 comprising the amino acid sequence set forth in SEQ ID NO: 96 (or a variant thereof);
(m) an HCVR that comprises: an HCDR1 comprising the amino acid sequence set forth in SEQ ID NO: 98 (or a variant thereof), an HCDR2 comprising the amino acid sequence set forth in SEQ ID NO: 99 (or a variant thereof), and an HCDR3 comprising the amino acid sequence set forth in SEQ ID NO: 100 (or a variant thereof); and
an LCVR that comprises: an LCDR1 comprising the amino acid sequence set forth in SEQ ID NO: 102 (or a variant thereof), an LCDR2 comprising the amino acid sequence set forth in SEQ ID NO: 103 (or a variant thereof), and an LCDR3 comprising the amino acid sequence set forth in SEQ ID NO: 104 (or a variant thereof);
(n) an HCVR that comprises: an HCDR1 comprising the amino acid sequence set forth in SEQ ID NO: 106 (or a variant thereof), an HCDR2 comprising the amino acid sequence set forth in SEQ ID NO: 107 (or a variant thereof), and an HCDR3 comprising the amino acid sequence set forth in SEQ ID NO: 108 (or a variant thereof); and
an LCVR that comprises: an LCDR1 comprising the amino acid sequence set forth in SEQ ID NO: 110 (or a variant thereof), an LCDR2 comprising the amino acid sequence set forth in SEQ ID NO: 111 (or a variant thereof), and an LCDR3 comprising the amino acid sequence set forth in SEQ ID NO: 112 (or a variant thereof);
(o) an HCVR that comprises: an HCDR1 comprising the amino acid sequence set forth in SEQ ID NO: 114 (or a variant thereof), an HCDR2 comprising the amino acid sequence set forth in SEQ ID NO: 115 (or a variant thereof), and an HCDR3 comprising the amino acid sequence set forth in SEQ ID NO: 116 (or a variant thereof); and
an LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 102 (or a variant thereof);
(p) an HCVR that comprises: an HCDR1 comprising the amino acid sequence set forth in SEQ ID NO: 122 (or a variant thereof), an HCDR2 comprising the amino acid sequence set forth in SEQ ID NO: 123 (or a variant thereof), and an HCDR3 comprising the amino acid sequence set forth in SEQ ID NO: 124 (or a variant thereof); and
an LCVR that comprises: an LCDR1 comprising the amino acid sequence set forth in SEQ ID NO: 126 (or a variant thereof), an LCDR2 comprising the amino acid sequence set forth in SEQ ID NO: 127 (or a variant thereof), and an LCDR3 comprising the amino acid sequence set forth in SEQ ID NO: 128 (or a variant thereof);
(q) an HCVR that comprises: an HCDR1 comprising the amino acid sequence set forth in SEQ ID NO: 130 (or a variant thereof), an HCDR2 comprising the amino acid sequence set forth in SEQ ID NO: 131 (or a variant thereof), and an HCDR3 comprising the amino acid sequence set forth in SEQ ID NO: 132 (or a variant thereof); and
an LCVR that comprises: an LCDR1 comprising the amino acid sequence set forth in SEQ ID NO: 134 (or a variant thereof), an LCDR2 comprising the amino acid sequence set forth in SEQ ID NO: 135 (or a variant thereof), and an LCDR3 comprising the amino acid sequence set forth in SEQ ID NO: 136 (or a variant thereof);
(r) an HCVR that comprises: an HCDR1 comprising the amino acid sequence set forth in SEQ ID NO: 138 (or a variant thereof), an HCDR2 comprising the amino acid sequence set forth in SEQ ID NO: 139 (or a variant thereof), and an HCDR3 comprising the amino acid sequence set forth in SEQ ID NO: 140 (or a variant thereof); and
an LCVR that comprises: an LCDR1 comprising the amino acid sequence set forth in SEQ ID NO: 142 (or a variant thereof), an LCDR2 comprising the amino acid sequence set forth in SEQ ID NO: 143 (or a variant thereof), and an LCDR3 comprising the amino acid sequence set forth in SEQ ID NO: 144 (or a variant thereof);
(s) an HCVR that comprises: an HCDR1 comprising the amino acid sequence set forth in SEQ ID NO: 431 (or a variant thereof), an HCDR2 comprising the amino acid sequence set forth in SEQ ID NO: 433 (or a variant thereof), and an HCDR3 comprising the amino acid sequence set forth in SEQ ID NO: 435 (or a variant thereof); and
an LCVR that comprises: an LCDR1 comprising the amino acid sequence set forth in SEQ ID NO: 439 (or a variant thereof), an LCDR2 comprising the amino acid sequence YNS (or a variant thereof), and an LCDR3 comprising the amino acid sequence set forth in SEQ ID NO: 441 (or a variant thereof); or
(t) an HCVR that comprises: an HCDR1 comprising the amino acid sequence set forth in SEQ ID NO: 453 (or a variant thereof), an HCDR2 comprising the amino acid sequence set forth in SEQ ID NO: 455 (or a variant thereof), and an HCDR3 comprising the amino acid sequence set forth in SEQ ID NO: 457 (or a variant thereof); and
an LCVR that comprises: an LCDR1 comprising the amino acid sequence set forth in SEQ ID NO: 461 (or a variant thereof), an LCDR2 comprising the amino acid sequence RNN (or a variant thereof), and an LCDR3 comprising the amino acid sequence set forth in SEQ ID NO: 463 (or a variant thereof).

4. The antigen-binding protein of any one of claims 1-3 which comprises:
(1) an HCVR that comprises the amino acid sequence set forth in SEQ ID NO: 1 (or a variant thereof); and an LCVR that comprises the amino acid sequence set forth in SEQ ID NO: 5 (or a variant thereof);
(2) an HCVR that comprises the amino acid sequence set forth in SEQ ID NO: 9; and an LCVR that comprises the amino acid sequence set forth in SEQ ID NO:
13 (or a variant thereof);
(3) an HCVR that comprises the amino acid sequence set forth in SEQ ID NO: 17; and an LCVR that comprises the amino acid sequence set forth in SEQ ID NO:
21 (or a variant thereof);
(4) an HCVR that comprises the amino acid sequence set forth in SEQ ID NO: 25 (or a variant thereof); and an LCVR that comprises the amino acid sequence set forth in SEQ ID NO: 29 (or a variant thereof);
(5) an HCVR that comprises the amino acid sequence set forth in SEQ ID NO: 33 (or a variant thereof); and an LCVR that comprises the amino acid sequence set forth in SEQ ID NO: 37 (or a variant thereof);
(6) an HCVR that comprises the amino acid sequence set forth in SEQ ID NO: 41 (or a variant thereof); and an LCVR that comprises the amino acid sequence set forth in SEQ ID NO: 45 (or a variant thereof);
(7) an HCVR that comprises the amino acid sequence set forth in SEQ ID NO: 49 (or a variant thereof); and an LCVR that comprises the amino acid sequence set forth in SEQ ID NO: 53 (or a variant thereof);
(8) an HCVR that comprises the amino acid sequence set forth in SEQ ID NO: 57 (or a variant thereof); and an LCVR that comprises the amino acid sequence set forth in SEQ ID NO: 61 (or a variant thereof);
(9) an HCVR that comprises the amino acid sequence set forth in SEQ ID NO: 65 (or a variant thereof); and an LCVR that comprises the amino acid sequence set forth in SEQ ID NO: 69 (or a variant thereof);
(10) an HCVR that comprises the amino acid sequence set forth in SEQ ID NO: 73 (or a variant thereof); and an LCVR that comprises the amino acid sequence set forth in SEQ ID NO: 77 (or a variant thereof);
(11) an HCVR that comprises the amino acid sequence set forth in SEQ ID NO: 81 (or a variant thereof); and an LCVR that comprises the amino acid sequence set forth in SEQ ID NO: 85 (or a variant thereof); or
(12) an HCVR that comprises the amino acid sequence set forth in SEQ ID NO: 89 (or a variant thereof); and an LCVR that comprises the amino acid sequence set forth in SEQ ID NO: 93 (or a variant thereof)
(13) an HCVR that comprises the amino acid sequence set forth in SEQ ID NO: 97 (or a variant thereof); and an LCVR that comprises the amino acid sequence set forth in SEQ ID NO: 101 (or a variant thereof);
(14) an HCVR that comprises the amino acid sequence set forth in SEQ ID NO: 105 (or a variant thereof); and an LCVR that comprises the amino acid sequence set forth in SEQ ID NO: 109 (or a variant thereof);
(15) an HCVR that comprises the amino acid sequence set forth in SEQ ID NO: 113 (or a variant thereof); and an LCVR that comprises the amino acid sequence set forth in SEQ ID NO: 117 (or a variant thereof);
(16) an HCVR that comprises the amino acid sequence set forth in SEQ ID NO: 121 (or a variant thereof); and an LCVR that comprises the amino acid sequence set forth in SEQ ID NO: 125 (or a variant thereof);
(17) an HCVR that comprises the amino acid sequence set forth in SEQ ID NO: 129 (or a variant thereof); and an LCVR that comprises the amino acid sequence set forth in SEQ ID NO: 133 (or a variant thereof);
(18) an HCVR that comprises the amino acid sequence set forth in SEQ ID NO: 137 (or a variant thereof); and an LCVR that comprises the amino acid sequence set forth in SEQ ID NO: 141 (or a variant thereof);
(19) an HCVR that comprises the amino acid sequence set forth in SEQ ID NO: 429 (or a variant thereof); and an LCVR that comprises the amino acid sequence set forth in SEQ ID NO: 437 (or a variant thereof); or
(20) an HCVR that comprises the amino acid sequence set forth in SEQ ID NO: 451 (or a variant thereof); and an LCVR that comprises the amino acid sequence set forth in SEQ ID NO: 459 (or a variant thereof).

5. The antigen-binding protein of any one of claims 1-4 which comprises:
(a) a heavy chain that comprises the amino acid sequence set forth in SEQ ID NO: 145 (or a variant thereof), and a light chain that comprises the amino acid sequence set forth in SEQ ID NO: 146 (or a variant thereof);
(b) a heavy chain that comprises the amino acid sequence set forth in SEQ ID NO: 147 (or a variant thereof), and a light chain that comprises the amino acid sequence set forth in SEQ ID NO: 148 (or a variant thereof);
(c) a heavy chain that comprises the amino acid sequence set forth in SEQ ID NO: 149 (or a variant thereof), and a light chain that comprises the amino acid sequence set forth in SEQ ID NO: 150 (or a variant thereof);
(d) a heavy chain that comprises the amino acid sequence set forth in SEQ ID NO: 151 (or a variant thereof), and a light chain that comprises the amino acid sequence set forth in SEQ ID NO: 152 (or a variant thereof);
(e) a heavy chain that comprises the amino acid sequence set forth in SEQ ID NO: 153 (or a variant thereof), and a light chain that comprises the amino acid sequence set forth in SEQ ID NO: 154 (or a variant thereof);
(f) a heavy chain that comprises the amino acid sequence set forth in SEQ ID NO: 155 (or a variant thereof), and a light chain that comprises the amino acid sequence set forth in SEQ ID NO: 156 (or a variant thereof);
(g) a heavy chain that comprises the amino acid sequence set forth in SEQ ID NO: 157 (or a variant thereof), and a light chain that comprises the amino acid sequence set forth in SEQ ID NO: 158 (or a variant thereof);
(h) a heavy chain that comprises the amino acid sequence set forth in SEQ ID NO: 159 (or a variant thereof), and a light chain that comprises the amino acid sequence set forth in SEQ ID NO: 160 (or a variant thereof);
(i) a heavy chain that comprises the amino acid sequence set forth in SEQ ID NO: 161 (or a variant thereof), and a light chain that comprises the amino acid sequence set forth in SEQ ID NO: 162 (or a variant thereof);
(j) a heavy chain that comprises the amino acid sequence set forth in SEQ ID NO: 163 (or a variant thereof), and a light chain that comprises the amino acid sequence set forth in SEQ ID NO: 164 (or a variant thereof);
(k) a heavy chain that comprises the amino acid sequence set forth in SEQ ID NO: 165 (or a variant thereof), and a light chain that comprises the amino acid sequence set forth in SEQ ID NO: 166 (or a variant thereof);
(l) a heavy chain that comprises the amino acid sequence set forth in SEQ ID NO: 167 (or a variant thereof), and a light chain that comprises the amino acid sequence set forth in SEQ ID NO: 168 (or a variant thereof);
(m) a heavy chain that comprises the amino acid sequence set forth in SEQ ID NO: 169 (or a variant thereof), and a light chain that comprises the amino acid sequence set forth in SEQ ID NO: 170 (or a variant thereof);
(n) a heavy chain that comprises the amino acid sequence set forth in SEQ ID NO: 171 (or a variant thereof), and a light chain that comprises the amino acid sequence set forth in SEQ ID NO: 172 (or a variant thereof);
(o) a heavy chain that comprises the amino acid sequence set forth in SEQ ID NO: 173 (or a variant thereof), and a light chain that comprises the amino acid sequence set forth in SEQ ID NO: 174 (or a variant thereof);
(p) a heavy chain that comprises the amino acid sequence set forth in SEQ ID NO: 175 (or a variant thereof), and a light chain that comprises the amino acid sequence set forth in SEQ ID NO: 176 (or a variant thereof);
(q) a heavy chain that comprises the amino acid sequence set forth in SEQ ID NO: 177 (or a variant thereof), and a light chain that comprises the amino acid sequence set forth in SEQ ID NO: 178 (or variant thereof);
(r) a heavy chain that comprises the amino acid sequence set forth in SEQ ID NO: 179 (or a variant thereof), and a light chain that comprises the amino acid sequence set forth in SEQ ID NO: 180 (or a variant thereof);
(s) a heavy chain that comprises the amino acid sequence set forth in SEQ ID NO: 443 (or a variant thereof), and a light chain that comprises the amino acid sequence set forth in SEQ ID NO: 447 (or a variant thereof); or
(t) a heavy chain that comprises the amino acid sequence set forth in SEQ ID NO: 465 (or a variant thereof), and a light chain that comprises the amino acid sequence set forth in SEQ ID NO: 469 (or a variant thereof).

6. An antigen-binding protein that binds to the same epitope on CACNG1 as an antibody comprising an HCVR/LCVR amino acid sequence pair as set forth in Table 1-1.

7. An antigen-binding protein that competes for binding to CACNG1 with an antibody comprising an HCVR/LCVR amino acid sequence pair as set forth in Table 1-1.

8. The antigen-binding protein of any one of claims 1-7, wherein the antigen-binding protein comprises an antibody or antigen-binding fragment thereof.

9. The antigen-binding protein of any one of claims 1-7, wherein the antigen-binding protein is a humanized antibody or antigen binding fragment thereof, a human antibody or antigen binding fragment thereof, a murine antibody or antigen binding fragment thereof, a chimeric antibody or antigen binding fragment thereof, a monovalent Fab′, a divalent Fab2, an F(ab)′3 fragment, a single-chain fragment variable (scFv), a bis-scFv, a (scFv)2, a diabody, a minibody, a nanobody, a triabody, a tetrabody, a disulfide stabilized Fv protein (dsFv), a single-domain antibody (sdAb), an Ig NAR, a single heavy chain antibody, a bispecific antibody or binding fragment thereof, a bi-specific T-cell engager (BiTE), a trispecific antibody, or a chemically modified derivative thereof.

10. The antigen-binding protein of claim 9, wherein the antigen-binding protein comprises a fragment antigen-binding region (Fab).

11. The antigen-binding protein of claim 9, wherein the antigen-binding protein comprises a single chain fragment variable (scFv).

12. The antigen-binding protein of claim 11, wherein the scFv comprises variable regions arranged in the following orientation from N-terminus to C-terminus: HCVR-LCVR.

13. The antigen-binding protein of claim 11, wherein the scFv comprises variable regions arranged in the following orientation from N-terminus to C-terminus: LCVR-HCVR.

14. The antigen-binding protein of any one of claims 11-13, wherein the variable regions in the scFv are connected by a linker.

15. The antigen-binding protein of claim 14, wherein the linker is a peptide linker.

16. The antigen-binding protein of claim 15, wherein the peptide linker is -(GGGGS)ₙ-(SEQ ID NO: 411), wherein n is any integral selected from 1-10.

17. The antigen-binding protein of any one of claims 1-16, wherein the antigen-binding protein binds specifically to human CACNG1.

18. The antigen-binding protein of claim 17, wherein the antigen-binding protein binds to human hCACNG1 with a K_{D} of about 1×10⁻⁷ M or a stronger affinity.

19. The antigen-binding protein of claim 18, wherein the anti-hCACNG1 antibody or antigen-binding fragment thereof binds to hCACNG1 with a K_{D} of about 1×10⁻⁷ to about 1×10⁻¹⁰.

20. The antigen-binding protein of claim 18, wherein the anti-hCACNG1 antibody or antigen-binding fragment thereof binds to hCACNG1 with a K_{D} of about 5×10⁻⁹ to about 1×10⁻¹⁰.

21. An isolated polynucleotide encoding the antigen-binding protein of any one of claims 1-20.

22. A vector comprising the isolated polynucleotide of claim 21.

23. A host cell comprising the antigen-binding protein of any one of claims 1-20, the isolated polynucleotide of claim 21, or the vector of claim 22.

24. The host cell of claim 23, which is a Chinese hamster ovary (CHO) cell.

25. A protein-drug conjugate comprising an antigen-binding protein that binds specifically to Calcium Voltage-Gated Channel Auxiliary Subunit Gamma 1 (CACNG1) and is conjugated to a molecular cargo.

26. The protein-drug conjugate of claim 25, wherein the antigen-binding protein comprises the antigen-binding protein of any one of claims 1-20.

27. The protein-drug conjugate of claim 25 or 26, wherein the antigen-binding protein and the molecular cargo are conjugated via a linker.

28. The protein-drug conjugate of any one of claims 25-27, wherein the molecular cargo comprises a polynucleotide molecule, a polypeptide molecule, a carrier, or a small molecule.

29. The protein-drug conjugate of claim 28, wherein the molecular cargo comprises a polynucleotide molecule.

30. The protein-drug conjugate of claim 29, wherein the polynucleotide molecule is an interfering nucleic acid molecule, a guide RNA, a ribozyme, an aptamer, a mixmer, a multimer, or an mRNA.

31. The protein-drug conjugate of claim 30, wherein the interfering nucleic acid molecule is an siRNA, an shRNA, a miRNA, an antisense oligonucleotide, or a gapmer.

32. The protein-drug conjugate of claim 31, wherein the interfering nucleic acid molecule is an siRNA.

33. The protein-drug conjugate of claim 32, wherein the siRNA comprises a sense strand of 21 nucleotides in length.

34. The protein-drug conjugate of claim 32 or 33, wherein the siRNA comprises an antisense strand of 23 nucleotides in length.

35. The protein-drug conjugate of any one of claims 32-34, wherein the siRNA comprises two phosphorothioate linkages at the first and second internucleoside linkages at the 5′ end of the sense strand.

36. The protein-drug conjugate of any one of claims 32-35, wherein the siRNA comprises two phosphorothioate linkages at the first and second internucleoside linkages at the 3′ and/or 5′ ends of the antisense strand.

37. The protein-drug conjugate of claim 31, wherein the interfering nucleic acid is an antisense oligonucleotide.

38. The protein-drug conjugate of claim 30, wherein the polynucleotide molecule is a guide RNA.

39. The protein-drug conjugate of any one of claims 29-38, wherein the polynucleotide molecule targets a gene or gene product associated with a skeletal muscle disease or disorder.

40. The protein-drug conjugate of claim 39, wherein the gene or gene product associated with a skeletal muscle disease or disorder is Double Homeobox 4 (DUX4), myotonic dystrophy protein kinase (DMPK), dystrophin (DMD), F-Box Only Protein 32 (FBX032), Tripartite Motif Containing 63 (TRIM63), Inhibin Subunit Beta A (INHBA), Myostatin (MSTN), Myocyte Enhancer Factor 2D (MEF2D), KLF Transcription Factor 15 (KLF15), Mediator Complex Subunit 1 (MED1), Mediator Complex Subunit 13 (MED13), Protein Phosphatase 1 Regulatory Subunit 3A (PPP1R3A), Myosin Light Chain Kinase (MLCK1), Activin A Receptor Type 1B (ACVR1B), Type II SH2-domain-containing inositol 5-phosphatase (SHIP2), or a gene disclosed in Table 1-3.

41. The protein-drug conjugate of any one of claims 29-40, wherein the polynucleotide molecule comprises one or more modified nucleotides.

42. The protein-drug conjugate of claim 28, wherein the molecular cargo comprises a polypeptide molecule.

43. The protein-drug conjugate of claim 42, wherein the polypeptide molecule is an enzyme or an antigen-binding protein that binds to a target other than CACNG1.

44. The protein-drug conjugate of claim 42 or 43, wherein the polypeptide molecule is associated with a skeletal muscle disease or disorder.

45. The protein-drug conjugate of claim 28, wherein the molecular cargo comprises a small molecule.

46. The protein drug conjugate of claim 45, wherein the small molecule is an androgen, a glucocorticoid, a β2-adrenergic receptor agonist, rapamycin or an analog thereof, a MAPK inhibitor, or a histone deacetylase inhibitor.

47. The protein-drug conjugate of claim 45 or 46, wherein the small molecule is an androgen.

48. The protein-drug-conjugate of claim 47, wherein the androgen is dihydrotestosterone (DHT).

49. The protein-drug conjugate of claim 45 or 46, wherein the small molecule is a glucocorticoid.

50. The protein-drug conjugate of claim 49, wherein the glucocorticoid is budesonide.

51. The protein-drug conjugate of any one of claims 28 and 45-50, wherein the antigen-binding protein and the small molecule are conjugated via a valine-citrulline para-aminobenzylcarbamate (VC-PAB) and/or a glutamic acid-valine-citrulline para-aminobenzylcarbamate (EVC-PAB) linker.

52. The protein-drug conjugate of any one of claims 25-51, for use in treating, preventing, or reducing the likelihood of a skeletal muscle disease or disorder.

53. The protein-drug conjugate of any one of claims 39, 40, 44, and 52, wherein the skeletal muscle disease or disorder is a muscular dystrophy, a muscular atrophy, an inflammatory myopathy, a disease of the peripheral nerve, a disease of the neuromuscular junction, a metabolic disease of the muscle, central core disease, hyperthyroid myopathy, myotonia congenita, myotubular myopathy, Nemaline myopathy, paramyotonia congenita, periodic paralysis-hypokalemic-hyperkalemic, centronuclear myopathy, Laing distal myopathy, myofibrillar myopathy, or a disease or disorder disclosed in Tables 1-3.

54. The protein-drug conjugate of claim 53, wherein the muscular dystrophy is Duchenne muscular dystrophy (DMD), Becker muscular dystrophy (BMD), a congenital muscular dystrophy, a distal muscular dystrophy, Emery-Dreifuss muscular dystrophy, a facioscapulohumeral muscular dystrophy, a Limb-Girdle muscular dystrophy, a myotonic muscular dystrophy, or an oculopharyngeal muscular dystrophy.

55. The protein-drug conjugate of claim 53, wherein the muscular atrophy is a spinal muscular atrophy, or a muscular atrophy induced by cancer cachexia, disuse, heart failure, chronic obstructive pulmonary disease, or a chronic infection.

56. The protein-drug conjugate of claim 55, wherein the spinal muscular atrophy is Amyotrophic Lateral Sclerosis (ALS), infantile progressive spinal muscular atrophy, intermediate spinal muscular atrophy, juvenile spinal muscular atrophy, or adult spinal muscular atrophy.

57. The protein-drug conjugate of claim 53, wherein the inflammatory myopathy is dermatomyositis, polymyositis, or inclusion body myositis.

58. The protein-drug conjugate of claim 53, wherein the disease of the peripheral nerve is Charcot-Marie tooth disease, Dejerine-Sottas disease, or Friedreich's ataxia.

59. The protein-drug conjugate of claim 53, wherein the disease of the neuromuscular junction is Myasthenia gravis, Lambert-Eaton syndrome, or botulism.

60. The protein-drug conjugate of claim 53, wherein the metabolic disease of the muscle is acid maltase deficiency, carnitine deficiency, carnitine palmityl transferase deficiency, debrancher enzyme deficiency, lactate dehydrogenase deficiency, mitochondrial myopathy, myoadenylate deaminase deficiency, phosphorylase deficiency, phosphofructokinase deficiency, or phosphoglycerate kinase deficiency.

61. The protein-drug conjugate of any one of claims 42-44, wherein the polypeptide molecule is Double Homeobox 4 (DUX4), myotonic dystrophy protein kinase (DMPK), dystrophin (DMD), F-Box Only Protein 32 (FBX032), Tripartite Motif Containing 63 (TRIM63), Inhibin Subunit Beta A (INHBA), Myostatin (MSTN), Myocyte Enhancer Factor 2D (MEF2D), KLF Transcription Factor 15 (KLF15), Mediator Complex Subunit 1 (MED1), Mediator Complex Subunit 13 (MED13), Protein Phosphatase 1 Regulatory Subunit 3A (PPP1 R3A), Myosin Light Chain Kinase (MLCK1), Activin A Receptor Type 1B (ACVR1 B), Type-II SH2-domain-containing inositol 5-phosphatase (SHIP2), or a protein disclosed in Tables 1-3.

62. The protein-drug conjugate of claim 28, wherein the molecular cargo comprises a carrier.

63. The protein-drug conjugate of claim 62, wherein the carrier is a lipid-based carrier.

64. The protein-drug conjugate of claim 63, wherein the lipid-based carrier is a lipid nanoparticle (LNP), a liposome, a lipidoid, or a lipoplex.

65. The protein-drug conjugate of claim 64, wherein the lipid-based carrier is a lipid nanoparticle (LNP).

66. The protein-drug conjugate of claim 65, wherein the LNP further comprises a polynucleotide molecule and/or a polypeptide molecule.

67. The protein-drug conjugate of claim 65 or 66, wherein the LNP comprises one or more components of a gene editing system.

68. The protein-drug conjugate of claim 67, wherein the LNP comprises:
(a) a Cas nuclease, or a nucleic acid encoding the Cas nuclease, and/or
(b) a guide RNA, or one or more DNAs encoding the guide RNA.

69. The protein-drug conjugate of claim 68, wherein the Cas nuclease is a Cas9 protein.

70. The protein-drug conjugate of claim 69, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

71. The protein-drug conjugate of any one of claims 68-70, wherein the nucleic acid encoding the Cas nuclease is codon-optimized for expression in a mammalian cell.

72. The protein-drug conjugate of claim 71, wherein the nucleic acid encoding the Cas nuclease is codon-optimized for expression in a human cell.

73. The protein-drug conjugate of any one of claims 68-72, wherein the nucleic acid encoding the Cas nuclease is an mRNA.

74. The protein-drug conjugate of any one of claims 30, 38-41, and 68-73, wherein the guide RNA is a single guide RNA (sgRNA).

75. The protein-drug conjugate of claim 67, wherein the LNP comprises a zinc finger nuclease (ZFN) or a transcription activator-like effector nuclease (TALEN).

76. The protein-drug conjugate of any one of claims 65-75, wherein the LNP comprises a cationic lipid, a neutral lipid, a helper lipid, a stealth lipid, or any combination thereof.

77. The protein-drug conjugate of claim 76, wherein the neutral lipid is distearoylphosphatidylcholine (DSPC).

78. The protein-drug conjugate of claim 76, wherein the helper lipid is cholesterol.

79. The protein-drug conjugate of claim 76, wherein the stealth lipid is PEG2k-DMG.

80. A pharmaceutical composition comprising the antigen-binding protein of any one of claims 1-20, the isolated polynucleotide of claim 21, the vector of claim 22, or the protein-drug conjugate of any one of claims 25-79, and a pharmaceutically acceptable carrier.

81. A composition or kit comprising the antigen-binding protein of any one of claims 1-20, the isolated polynucleotide of claim 21, the vector of claim 22, the protein-drug conjugate of any one of claims 25-79, or the pharmaceutical composition of claim 80, and a further therapeutic agent.

82. A complex comprising the antigen-binding protein of any one of claims 1-20 or the protein-drug conjugate of any one of claims 25-79 bound to Calcium Voltage-Gated Channel Auxiliary Subunit Gamma 1 (CACNG1).

83. A method for making the antigen-binding protein of any one of claims 1-20, comprising culturing a host cell comprising a polynucleotide that encodes the antigen-binding protein in a culture medium under conditions favorable for expression of the antigen-binding protein.

84. The method of claim 83, comprising the steps:
(a) introducing said polynucleotide into a host cell;
(b) culturing the host cell under conditions favorable for expression of the antigen-binding protein;
(c) optionally, isolating the antigen-binding protein from the culture medium and/or host cell; and
(d) optionally, conjugating the antigen-binding protein to a molecular cargo.

85. An antigen-binding protein which is produced by or obtainable by the method of claim 83 or claim 84.

86. A method for making a protein-drug conjugate of any one of claims 25-79 comprising:
(a) contacting the antigen-binding protein, with the molecular cargo under the conditions favorable for conjugation of the antigen-binding protein to the molecular cargo; and
(b) optionally, isolating the protein-drug conjugate produced in step (a).

87. A method for making a protein-drug conjugate of any one of claims 42-44 and 61, wherein the molecular cargo comprises a polypeptide molecule, comprising:
(a) culturing a host cell comprising a polynucleotide encoding the protein-drug conjugate under conditions that allow expression of the protein-drug conjugate; and
(b) optionally, isolating the protein-drug conjugate produced in step (a).

88. A protein-drug conjugate produced by or obtainable by the method of claim 86 or 87.

89. A vessel or injection device comprising the antigen-binding protein of any one of claims 1-20 and 85, the isolated polynucleotide of claim 21, the vector of claim 22, or the protein-drug conjugate of any one of claims 25-79 and 88.

90. A method for imaging skeletal muscle in a subject in need thereof, comprising introducing the antigen-binding protein of any one of claims 1-20 and 85 into the body of the subject, wherein the antigen-binding protein is conjugated to a detectable biosensor or a radioactive isotope.

91. The method of claim 90, wherein the radioactive isotope comprises a radionuclide.

92. The method of claim 90 or 91, wherein the antigen-binding protein conjugated to a detectable biosensor or a radioactive isotope is introduced to the subject via intramuscular, intravenous or subcutaneous administration.

93. A method for causing internalization of a small molecule by a myofiber, comprising contacting the myofiber with the antigen-binding protein of any one of claims 1-20 and 85, and wherein the antigen-binding protein is conjugated to a small molecule.

94. The method of claim 93, wherein the small molecule is an androgen, a glucocorticoid, a β2-adrenergic receptor agonist, rapamycin or an analog thereof, a MAPK inhibitor, or a histone deacetylase inhibitor.

95. The method of claim 93 or 94, wherein the small molecule is an androgen.

96. The protein-drug-conjugate of claim 95, wherein the androgen is dihydrotestosterone (DHT).

97. The protein-drug conjugate of claim 93 or 94, wherein the small molecule is a glucocorticoid.

98. The protein-drug conjugate of claim 97, wherein the glucocorticoid is budesonide.

99. The method of claim 93, wherein the small molecule comprises a detectable biosensor or a radioactive isotope.

100. The method of claim 93, wherein the radioactive isotope comprises a radionuclide.

101. The method of any one of claims 93-100, wherein the contacting comprises administering intramuscularly, intravenously or subcutaneously to a subject in need thereof the antigen-binding protein conjugated to the small molecule.

102. The method of any one of claims 93-100, wherein the contacting comprises culturing the myofiber in vitro with the antigen-binding protein conjugated to the small molecule.

103. The method of any one of claims 93-102, wherein the antigen-binding protein is conjugated to the small molecule via a valine-citrulline para-aminobenzylcarbamate (VC-PAB) and/or a glutamic acid-valine-citrulline para-aminobenzylcarbamate (EVC-PAB) linker.

104. A method for administering the antigen-binding protein of any one of claims 1-20 and 85, the isolated polynucleotide of claim 21, the vector of claim 22, or the protein-drug conjugate of any one of claims 25-79 and 88 to a subject in need thereof, said method comprising introducing the antigen-binding protein, the polynucleotide, the vector, or the protein-drug conjugate into the body of the subject.

105. The method of claim 104, wherein the antigen-binding protein, the polynucleotide, the vector, or the protein-drug conjugate is introduced into the body of the subject via intramuscular, subcutaneous, or intravenous administration.

106. A method for delivering a molecular cargo to a skeletal muscle tissue and/or cell in the body of a subject in need thereof comprising administering to the subject the protein-drug conjugate of any one of claims 25-79 and 88 or the pharmaceutical composition of claim 80.

107. A method for treating, preventing, reducing the likelihood of a skeletal muscle disease or disorder in a subject in need thereof comprising administering to the subject a therapeutically-effective amount of the antigen-binding protein of any one of claims 1-20 and 85, the isolated polynucleotide of claim 21, the vector of claim 22, the protein-drug conjugate of any one of claims 25-79 and 88, or the pharmaceutical composition of claim 80.

108. The method of claim 107, wherein the antigen-binding protein, the polynucleotide, the vector, or the protein-drug conjugate is administered via intramuscular, subcutaneous, or intravenous administration.

109. The method of claim 107 or 108, wherein the skeletal muscle disease or disorder is a muscular dystrophy, a muscular atrophy, an inflammatory myopathy, a disease of the peripheral nerve, a disease of the neuromuscular junction, a metabolic disease of the muscle, central core disease, hyperthyroid myopathy, myotonia congenita, myotubular myopathy, Nemaline myopathy, paramyotonia congenita, periodic paralysis-hypokalemic-hyperkalemic, centronuclear myopathy, Laing distal myopathy, myofibrillar myopathy, or a disease or disorder disclosed in Tables 1-3.

110. The method of claim 109, wherein the muscular dystrophy is Duchenne muscular dystrophy (DMD), Becker muscular dystrophy (BMD), a congenital muscular dystrophy, a distal muscular dystrophy, Emery-Dreifuss muscular dystrophy, a facioscapulohumeral muscular dystrophy, a Limb-Girdle muscular dystrophy, a myotonic muscular dystrophy, or an oculopharyngeal muscular dystrophy.

111. The method of claim 109, wherein the muscular atrophy is a spinal muscular atrophy, or a muscular atrophy induced by cancer cachexia, disuse, heart failure, chronic obstructive pulmonary disease, or a chronic infection.

112. The method of claim 111, wherein the spinal muscular atrophy is Amyotrophic Lateral Sclerosis (ALS), infantile progressive spinal muscular atrophy, intermediate spinal muscular atrophy, juvenile spinal muscular atrophy, or adult spinal muscular atrophy.

113. The method of claim 109, wherein the inflammatory myopathy is dermatomyositis, polymyositis, or inclusion body myositis.

114. The method of claim 109, wherein the disease of the peripheral nerve is Charcot-Marie tooth disease, Dejerine-Sottas disease, or Friedreich's ataxia.

115. The method of claim 109, wherein the disease of the neuromuscular junction is Myasthenia gravis, Lambert-Eaton syndrome, or botulism.

116. The method of claim 109, wherein the metabolic disease of the muscle is acid maltase deficiency, carnitine deficiency, carnitine palmityl transferase deficiency, debrancher enzyme deficiency, lactate dehydrogenase deficiency, mitochondrial myopathy, myoadenylate deaminase deficiency, phosphorylase deficiency, phosphofructokinase deficiency, or phosphoglycerate kinase deficiency.

117. The method of any one of claims 107-116, further comprising administering an additional treatment to the subject.

118. The method of claim 117, wherein the additional treatment comprises physical exercise.

119. The method of claim 117, wherein the additional treatment comprises administering a testosterone and/or a glucocorticoid.


SEQUENCE LISTING (474 sequences)

SEQ ID NO: 1 - protein, 119 aa, synthetic construct
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSS

SEQ ID NO: 2 - protein, 8 aa, synthetic construct
GITFRNYG

SEQ ID NO: 3 - protein, 8 aa, synthetic construct
MWYDGSNK

SEQ ID NO: 4 - protein, 14 aa, synthetic construct
ARRGTIRTAAPFDY

SEQ ID NO: 5 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 6 - protein, 6 aa, synthetic construct
QSISSY

SEQ ID NO: 7 - skipped

SEQ ID NO: 8 - protein, 10 aa, synthetic construct
QQSYSTPPIT

SEQ ID NO: 9 - protein, 121 aa, synthetic construct
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSS

SEQ ID NO: 10 - protein, 8 aa, synthetic construct
GITFRNYG

SEQ ID NO: 11 - protein, 8 aa, synthetic construct
MWYDGSNK

SEQ ID NO: 12 - protein, 14 aa, synthetic construct
ARRGHIATAAPFDY

SEQ ID NO: 13 - protein, 108 aa, synthetic construct
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIK

SEQ ID NO: 14 - protein, 6 aa, synthetic construct
QSISSY

SEQ ID NO: 15 - skipped

SEQ ID NO: 16 - protein, 10 aa, synthetic construct
QQSYSTPPIT

SEQ ID NO: 17 - protein, 121 aa, synthetic construct
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSS

SEQ ID NO: 18 - protein, 8 aa, synthetic construct
GFTFRSYG

SEQ ID NO: 19 - protein, 8 aa, synthetic construct
IWIDGNNI

SEQ ID NO: 20 - protein, 14 aa, synthetic construct
ARRLAITSAAPFDY

SEQ ID NO: 21 - protein, 108 aa, synthetic construct; identical to SEQ ID NO: 5

SEQ ID NO: 22 - protein, 6 aa, synthetic construct
QSISSY

SEQ ID NO: 23 - skipped

SEQ ID NO: 24 - protein, 10 aa, synthetic construct
QQSYSTPPIT

SEQ ID NO: 25 - protein, 126 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSS

SEQ ID NO: 26 - protein, 8 aa, synthetic construct
GYAFTTYG

SEQ ID NO: 27 - protein, 8 aa, synthetic construct
ISAYNGNT

SEQ ID NO: 28 - protein, 19 aa, synthetic construct
ARKGHYGSGTYYNPFGFDF

SEQ ID NO: 29 - protein, 108 aa, synthetic construct
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIK

SEQ ID NO: 30 - protein, 7 aa, synthetic construct
QSVSSSY

SEQ ID NO: 31 - skipped

SEQ ID NO: 32 - protein, 9 aa, synthetic construct
QQYYGSPWT

SEQ ID NO: 33 - protein, 120 aa, synthetic construct
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSS

SEQ ID NO: 34 - protein, 8 aa, synthetic construct
GLTLSDSA

SEQ ID NO: 35 - protein, 10 aa, synthetic construct
IRNKANRYAT

SEQ ID NO: 36 - protein, 11 aa, synthetic construct
TRNWKIFLFDY

SEQ ID NO: 37 - protein, 108 aa, synthetic construct
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIK

SEQ ID NO: 38 - protein, 7 aa, synthetic construct
QSVGSKY

SEQ ID NO: 39 - skipped

SEQ ID NO: 40 - protein, 9 aa, synthetic construct
QQYGSSPWT

SEQ ID NO: 41 - protein, 125 aa, synthetic construct
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSS

SEQ ID NO: 42 - protein, 8 aa, synthetic construct
GFTFNNYG

SEQ ID NO: 43 - protein, 8 aa, synthetic construct
ISGSGGTT

SEQ ID NO: 44 - protein, 18 aa, synthetic construct
GKGGYCSSSGCRHYGMDV

SEQ ID NO: 45 - protein, 110 aa, synthetic construct
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVL

SEQ ID NO: 46 - protein, 8 aa, synthetic construct
SSNIGNNY

SEQ ID NO: 47 - skipped

SEQ ID NO: 48 - protein, 11 aa, synthetic construct
AAWDDTLSGYV

SEQ ID NO: 49 - protein, 121 aa, synthetic construct
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSS

SEQ ID NO: 50 - protein, 8 aa, synthetic construct
GFTFTSHA

SEQ ID NO: 51 - protein, 8 aa, synthetic construct
ITGRGFDT

SEQ ID NO: 52 - protein, 14 aa, synthetic construct
AKGLYDSGNYYIDY

SEQ ID NO: 53 - protein, 111 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVL

SEQ ID NO: 54 - protein, 8 aa, synthetic construct
SSNIGNNY

SEQ ID NO: 55 - skipped

SEQ ID NO: 56 - protein, 12 aa, synthetic construct
GTWDLSLSFNWV

SEQ ID NO: 57 - protein, 119 aa, synthetic construct
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSS

SEQ ID NO: 58 - protein, 8 aa, synthetic construct
GGSFSGYY

SEQ ID NO: 59 - protein, 7 aa, synthetic construct
ILHSGRT

SEQ ID NO: 60 - protein, 13 aa, synthetic construct
AGRIAARHGWFDP

SEQ ID NO: 61 - protein, 107 aa, synthetic construct
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIK

SEQ ID NO: 62 - protein, 6 aa, synthetic construct
QDIRKW

SEQ ID NO: 63 - skipped

SEQ ID NO: 64 - protein, 9 aa, synthetic construct
QQANSFPFT

SEQ ID NO: 65 - protein, 116 aa, synthetic construct
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSS

SEQ ID NO: 66 - protein, 8 aa, synthetic construct
GDSINNYY

SEQ ID NO: 67 - protein, 7 aa, synthetic construct
IYYSGSA

SEQ ID NO: 68 - protein, 10 aa, synthetic construct
ARGAVKYFRH

SEQ ID NO: 69 - protein, 108 aa, synthetic construct
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIK

SEQ ID NO: 70 - protein, 7 aa, synthetic construct
QTINHNN

SEQ ID NO: 71 - skipped

SEQ ID NO: 72 - protein, 9 aa, synthetic construct
QQYGSLPLT

SEQ ID NO: 73 - protein, 119 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSS

SEQ ID NO: 74 - protein, 8 aa, synthetic construct
GFTFSTYG

SEQ ID NO: 75 - protein, 8 aa, synthetic construct
IWHDGSDK

SEQ ID NO: 76 - protein, 12 aa, synthetic construct
ARRGIRGTVFDH

SEQ ID NO: 77 - protein, 107 aa, synthetic construct
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIK

SEQ ID NO: 78 - protein, 6 aa, synthetic construct
QSISNK

SEQ ID NO: 79 - skipped

SEQ ID NO: 80 - protein, 9 aa, synthetic construct
QQYNSYSWT

SEQ ID NO: 81 - protein, 121 aa, synthetic construct
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSS

SEQ ID NO: 82 - protein, 8 aa, synthetic construct
GFTFSSYG

SEQ ID NO: 83 - protein, 8 aa, synthetic construct
IWIDGSNK

SEQ ID NO: 84 - protein, 14 aa, synthetic construct
ARRGGIVVAAPFDY

SEQ ID NO: 85 - protein, 108 aa, synthetic construct; identical to SEQ ID NO: 5

SEQ ID NO: 86 - protein, 6 aa, synthetic construct
QSISSY

SEQ ID NO: 87 - skipped

SEQ ID NO: 88 - protein, 10 aa, synthetic construct
QQSYSTPPIT

SEQ ID NO: 89 - protein, 126 aa, synthetic construct; identical to SEQ ID NO: 25

SEQ ID NO: 90 - protein, 8 aa, synthetic construct
GYAFTTYG

SEQ ID NO: 91 - protein, 8 aa, synthetic construct
ISAYNGNT

SEQ ID NO: 92 - protein, 19 aa, synthetic construct
ARKGHYGSGTYYNPFGFDF

SEQ ID NO: 93 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIK

SEQ ID NO: 94 - protein, 7 aa, synthetic construct
QSVSSSY

SEQ ID NO: 95 - skipped

SEQ ID NO: 96 - protein, 9 aa, synthetic construct
QQYYGSPWT

SEQ ID NO: 97 - protein, 119 aa, synthetic construct; identical to SEQ ID NO: 57

SEQ ID NO: 98 - protein, 8 aa, synthetic construct
GGSFSGYY

SEQ ID NO: 99 - protein, 7 aa, synthetic construct
ILHSGRT

SEQ ID NO: 100 - protein, 13 aa, synthetic construct
AGRIAARHGWFDP

SEQ ID NO: 101 - protein, 107 aa, synthetic construct; identical to SEQ ID NO: 61

SEQ ID NO: 102 - protein, 6 aa, synthetic construct
QDIRKW

SEQ ID NO: 103 - skipped

SEQ ID NO: 104 - protein, 9 aa, synthetic construct
QQANSFPFT

SEQ ID NO: 105 - protein, 116 aa, synthetic construct; identical to SEQ ID NO: 65

SEQ ID NO: 106 - protein, 8 aa, synthetic construct
GDSINNYY

SEQ ID NO: 107 - protein, 7 aa, synthetic construct
IYYSGSA

SEQ ID NO: 108 - protein, 10 aa, synthetic construct
ARGAVKYFRH

SEQ ID NO: 109 - protein, 108 aa, synthetic construct; identical to SEQ ID NO: 69

SEQ ID NO: 110 - protein, 7 aa, synthetic construct
QTINHNN

SEQ ID NO: 111 - skipped

SEQ ID NO: 112 - protein, 9 aa, synthetic construct
QQYGSLPLT

SEQ ID NO: 113 - protein, 119 aa, synthetic construct; identical to SEQ ID NO: 73

SEQ ID NO: 114 - protein, 8 aa, synthetic construct
GFTFSTYG

SEQ ID NO: 115 - protein, 8 aa, synthetic construct
IWHDGSDK

SEQ ID NO: 116 - protein, 12 aa, synthetic construct
ARRGIRGTVFDH

SEQ ID NO: 117 - protein, 107 aa, synthetic construct; identical to SEQ ID NO: 77

SEQ ID NO: 118 - protein, 6 aa, synthetic construct
QSISNK

SEQ ID NO: 119 - skipped

SEQ ID NO: 120 - protein, 9 aa, synthetic construct
QQYNSYSWT

SEQ ID NO: 121 - protein, 121 aa, synthetic construct; identical to SEQ ID NO: 49

SEQ ID NO: 122 - protein, 8 aa, synthetic construct
GFTFTSHA

SEQ ID NO: 123 - protein, 8 aa, synthetic construct
ITGRGFDT

SEQ ID NO: 124 - protein, 14 aa, synthetic construct
AKGLYDSGNYYIDY

SEQ ID NO: 125 - protein, 111 aa, synthetic construct; identical to SEQ ID NO: 53

SEQ ID NO: 126 - protein, 8 aa, synthetic construct
SSNIGNNY

SEQ ID NO: 127 - skipped

SEQ ID NO: 128 - protein, 12 aa, synthetic construct
GTWDLSLSFNWV

SEQ ID NO: 129 - protein, 121 aa, synthetic construct; identical to SEQ ID NO: 81

SEQ ID NO: 130 - protein, 8 aa, synthetic construct
GFTFSSYG

SEQ ID NO: 131 - protein, 8 aa, synthetic construct
IWIDGSNK

SEQ ID NO: 132 - protein, 14 aa, synthetic construct
ARRGGIVVAAPFDY

SEQ ID NO: 133 - protein, 108 aa, synthetic construct; identical to SEQ ID NO: 5

SEQ ID NO: 134 - protein, 6 aa, synthetic construct
QSISSY

SEQ ID NO: 135 - skipped

SEQ ID NO: 136 - protein, 10 aa, synthetic construct
QQSYSTPPIT

SEQ ID NO: 137 - protein, 119 aa, synthetic construct; identical to SEQ ID NO: 1

SEQ ID NO: 138 - protein, 8 aa, synthetic construct
GITFRNYG

SEQ ID NO: 139 - protein, 8 aa, synthetic construct
MWYDGSNK

SEQ ID NO: 140 - protein, 14 aa, synthetic construct
ARRGTIRTAAPFDY

SEQ ID NO: 141 - protein, 108 aa, synthetic construct; identical to SEQ ID NO: 5

SEQ ID NO: 142 - protein, 6 aa, synthetic construct
QSISSY

SEQ ID NO: 143 - skipped

SEQ ID NO: 144 - protein, 10 aa, synthetic construct
QQSYSTPPIT

SEQ ID NO: 145 - protein, 445 aa, synthetic construct
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 451 aa, synthetic construct
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 148 - protein, 215 aa, synthetic construct
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 149 - protein, 448 aa, synthetic construct
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 150 - protein, 215 aa, synthetic construct; identical to SEQ ID NO: 146

SEQ ID NO: 151 - protein, 450 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 152 - protein, 215 aa, synthetic construct
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 153 - protein, 447 aa, synthetic construct
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 154 - protein, 215 aa, synthetic construct
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 455 aa, synthetic construct
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 156 - protein, 217 aa, synthetic construct
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVLRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 157 - protein, 448 aa, synthetic construct
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 217 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 159 - protein, 446 aa, synthetic construct
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - protein, 214 aa, synthetic construct
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 443 aa, synthetic construct
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 215 aa, synthetic construct
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 446 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 164 - protein, 214 aa, synthetic construct
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 448 aa, synthetic construct
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 166 - protein, 215 aa, synthetic construct; identical to SEQ ID NO: 146

SEQ ID NO: 167 - protein, 453 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 449 aa, synthetic construct
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 214 aa, synthetic construct; identical to SEQ ID NO: 160

SEQ ID NO: 171 - protein, 446 aa, synthetic construct
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 215 aa, synthetic construct; identical to SEQ ID NO: 162

SEQ ID NO: 173 - protein, 449 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 214 aa, synthetic construct; identical to SEQ ID NO: 164

SEQ ID NO: 175 - protein, 451 aa, synthetic construct
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 217 aa, synthetic construct; identical to SEQ ID NO: 158

SEQ ID NO: 177 - protein, 451 aa, synthetic construct
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 215 aa, synthetic construct; identical to SEQ ID NO: 146

SEQ ID NO: 179 - protein, 449 aa, synthetic construct
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 215 aa, synthetic construct; identical to SEQ ID NO: 146

SEQ ID NO: 181 - DNA, 357 bp, synthetic construct
  source 1..357: mol_type = other DNA
caggtgcagctggtggagtctgggggaggcgtggtccagcctgggaggtccctgagactctcctgtacagcgtctggaatcaccttcagaaattatggcatgcactgggtccgccaggctccaggcaaggggctggagtgggtggcagttatgtggtatgatggaagtaataagtactatgcagactccgtgaagggccgtttcaccatctccggagacaattccaaggtgtatctgcaaatgaacagcctgagagccgaggacacggctgtatattactgtgcgagaaggggcactataagaacagctgccccttttgactactggggtcagggaaccctggtcaccgtctcctca

SEQ ID NO: 182 - DNA, 24 bp, synthetic construct
  source 1..24: mol_type = other DNA
ggaatcaccttcagaaattatggc

SEQ ID NO: 183 - DNA, 24 bp, synthetic construct
  source 1..24: mol_type = other DNA
atgtggtatgatggaagtaataag

SEQ ID NO: 184 - DNA, 42 bp, synthetic construct
  source 1..42: mol_type = other DNA
gcgagaaggggcactataagaacagctgccccttttgactac

SEQ ID NO: 185 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 186 - DNA, 18 bp, synthetic construct
  source 1..18: mol_type = other DNA
cagagcattagcagctat

SEQ ID NO: 187 - skipped

SEQ ID NO: 188 - DNA, 30 bp, synthetic construct
  source 1..30: mol_type = other DNA
caacagagttacagtacccctccgatcacc

SEQ ID NO: 189 - DNA, 1338 bp, synthetic construct
  source 1..1338: mol_type = other DNA
caggtgcagctggtggagtctgggggaggcgtggtccagcctgggaggtccctgagactctcctgtacagcgtctggaatcaccttcagaaattatggcatgcactgggtccgccaggctccaggcaaggggctggagtgggtggcagttatgtggtatgatggaagtaataagtactatgcagactccgtgaagggccgtttcaccatctccggagacaattccaaggtgtatctgcaaatgaacagcctgagagccgaggacacggctgtatattactgtgcgagaaggggcactataagaacagctgccccttttgactactggggtcagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 190 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 191 - DNA, 363 bp, synthetic construct
  source 1..363: mol_type = other DNA
caggtgcagttggtggagtctgggggaggcgtggtccagcctgggaggtccctgagactctcctgtgaagcgtctggaatcaccttcagaaactatggcatgcactgggtccgccaggctccaggcaaggggctggagtgggtggcagttatgtggtatgatggaagtaataaatactacgcagactccgtgaagggccgattcaccatctccagagacaattccaagaacacggtgtatctgcaaatgaacagcctgagagccgaagacacggctgtgtattactgtgcgagacggggtcatatagcaacagctgctccctttgactactggggccagggaaccctggtcaccgtctcctca

SEQ ID NO: 192 - DNA, 24 bp, synthetic construct
  source 1..24: mol_type = other DNA
ggaatcaccttcagaaactatggc

SEQ ID NO: 193 - DNA, 24 bp, synthetic construct
  source 1..24: mol_type = other DNA
atgtggtatgatggaagtaataaa

SEQ ID NO: 194 - DNA, 42 bp, synthetic construct
  source 1..42: mol_type = other DNA
gcgagacggggtcatatagcaacagctgctccctttgactac

SEQ ID NO: 195 - DNA, 324 bp, synthetic construct
  source 1..324: mol_type = other DNA
gacatccagatgacccagtctccatcctccctgtctgcatccgtaggagacagagtcaccatcagttgccgggcaagtcagagcattagtagttatttaaattggtatcagcagaaaccagggaaagcccctaaggtcctgatgtatgctgcatccagtttgcaaagtggggtcccatcaaggttcagtggcagtggatctgggacagatttcactctcaccatcagcagtctgcaacctgaggattttgcaacttactactgtcaacagagttacagtacccctccgatcaccttcggccaagggacacgactggagattaaa

SEQ ID NO: 196 - DNA, 18 bp, synthetic construct
  source 1..18: mol_type = other DNA
cagagcattagtagttat

SEQ ID NO: 197 - skipped

SEQ ID NO: 198 - DNA, 30 bp, synthetic construct; identical to SEQ ID NO: 188
  source 1..30: mol_type = other DNA

SEQ ID NO: 199 - DNA, 1356 bp, synthetic construct
  source 1..1356: mol_type = other DNA
caggtgcagttggtggagtctgggggaggcgtggtccagcctgggaggtccctgagactctcctgtgaagcgtctggaatcaccttcagaaactatggcatgcactgggtccgccaggctccaggcaaggggctggagtgggtggcagttatgtggtatgatggaagtaataaatactacgcagactccgtgaagggccgattcaccatctccagagacaattccaagaacacggtgtatctgcaaatgaacagcctgagagccgaagacacggctgtgtattactgtgcgagacggggtcatatagcaacagctgctccctttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 200 - DNA, 648 bp, synthetic construct
  source 1..648: mol_type = other DNA
gacatccagatgacccagtctccatcctccctgtctgcatccgtaggagacagagtcaccatcagttgccgggcaagtcagagcattagtagttatttaaattggtatcagcagaaaccagggaaagcccctaaggtcctgatgtatgctgcatccagtttgcaaagtggggtcccatcaaggttcagtggcagtggatctgggacagatttcactctcaccatcagcagtctgcaacctgaggattttgcaacttactactgtcaacagagttacagtacccctccgatcaccttcggccaagggacacgactggagattaaacgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 201 - DNA, 363 bp, synthetic construct
  source 1..363: mol_type = other DNA
caggtgcagctggtggagtctgggggaggcgtggtccagcctgggaggtccctgagactctcctgtacagcgtctggattcaccttccgttcctatggcatgcactgggtccgccaggctccaggcaaggggctggagtgggtgtcagttatttggattgatggaaataatatatactatgcagactccgtgaagggccgattcaccatctccagagacaattccaagaacacgctgtatctgcaaatggacagcctgagagccgaggacacggctgtttattactgtgcgagaagactggctataacatcagctgccccctttgactactggggccagggaaccctggtcaccgtctcctca

SEQ ID NO: 202 - DNA, 24 bp, synthetic construct
  source 1..24: mol_type = other DNA
ggattcaccttccgttcctatggc

SEQ ID NO: 203 - DNA, 24 bp, synthetic construct
  source 1..24: mol_type = other DNA
atttggattgatggaaataatata

SEQ ID NO: 204 - DNA, 42 bp, synthetic construct
  source 1..42: mol_type = other DNA
gcgagaagactggctataacatcagctgccccctttgactac

SEQ ID NO: 205 - DNA, 324 bp, synthetic construct; identical to SEQ ID NO: 185
  source 1..324: mol_type = other DNA

SEQ ID NO: 206 - DNA, 18 bp, synthetic construct
  source 1..18: mol_type = other DNA
cagagcattagcagctat

SEQ ID NO: 207 - skipped

SEQ ID NO: 208 - DNA, 30 bp, synthetic construct; identical to SEQ ID NO: 188
  source 1..30: mol_type = other DNA

SEQ ID NO: 209 - DNA, 1347 bp, synthetic construct
  source 1..1347: mol_type = other DNA
caggtgcagctggtggagtctgggggaggcgtggtccagcctgggaggtccctgagactctcctgtacagcgtctggattcaccttccgttcctatggcatgcactgggtccgccaggctccaggcaaggggctggagtgggtgtcagttatttggattgatggaaataatatatactatgcagactccgtgaagggccgattcaccatctccagagacaattccaagaacacgctgtatctgcaaatggacagcctgagagccgaggacacggctgtttattactgtgcgagaagactggctataacatcagctgccccctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 210 - DNA, 648 bp, synthetic construct; identical to SEQ ID NO: 190
  source 1..648: mol_type = other DNA

SEQ ID NO: 211 - DNA, 378 bp, synthetic construct
  source 1..378: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttca

SEQ ID NO: 212 - DNA, 24 bp, synthetic construct
  source 1..24: mol_type = other DNA
ggttacgccttcaccacctatggt

SEQ ID NO: 213 - DNA, 24 bp, synthetic construct
  source 1..24: mol_type = other DNA
atcagcgcttacaatggaaataca

SEQ ID NO: 214 - DNA, 57 bp, synthetic construct
  source 1..57: mol_type = other DNA
gcgagaaagggtcactatggttcggggacttattataacccctttggttttgatttt

SEQ ID NO: 215 - DNA, 326 bp, synthetic construct
  source 1..326: mol_type = other DNA
gaaattatgttgatgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactgacatcccagacaggttcagtggcagtgggtctgggacagacttcactctcaccatcagcagactggagcctgaagattttgcagtttatttctgtcagcagtattatggctcaccttggacgttcggccaagggaccaaggtggaaatcaagcg

SEQ ID NO: 216 - DNA, 21 bp, synthetic construct
  source 1..21: mol_type = other DNA
cagagtgttagcagcagctac

SEQ ID NO: 217 - skipped

SEQ ID NO: 218 - DNA, 27 bp, synthetic construct
  source 1..27: mol_type = other DNA
cagcagtattatggctcaccttggacg

SEQ ID NO: 219 - DNA, 1353 bp, synthetic construct
  source 1..1353: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagtccaactgggaggcaggaaatactttcacctgctctgtgttacatgagggcctgcacaaccaccatactgagaagtccctctcccactctcctggtaaatga

SEQ ID NO: 220 - DNA, 648 bp, synthetic construct
  source 1..648: mol_type = other DNA
gaaattatgttgatgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactgacatcccagacaggttcagtggcagtgggtctgggacagacttcactctcaccatcagcagactggagcctgaagattttgcagtttatttctgtcagcagtattatggctcaccttggacgttcggccaagggaccaaggtggaaatcaagcgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 221 - DNA, 360 bp, synthetic construct
  source 1..360: mol_type = other DNA
gaggtgcagctggtggagtctgggggaggcttggtccagccgggggggtccctgaaactctcctgtacagcctctgggttgaccctcagtgactctgctatgcactgggtccgccaggcttccgggaaagggctggagtgggttggccgtataagaaataaggctaataggtacgcgacagaatatgctgcgtcggtgaaaggcaggttcaccatttcaagagatgattcaaagaacacggcgtatctacaaatgaacagcctgaaaaccgaggacacggccgtgtattattgtactagaaactggaagattttcctctttgactactggggccagggaaccctggtcaccgtctcctca

SEQ ID NO: 222 - DNA, 24 bp, synthetic construct
  source 1..24: mol_type = other DNA
gggttgaccctcagtgactctgct

SEQ ID NO: 223 - DNA, 30 bp, synthetic construct
  source 1..30: mol_type = other DNA
ataagaaataaggctaataggtacgcgaca

SEQ ID NO: 224 - DNA, 33 bp, synthetic construct
  source 1..33: mol_type = other DNA
actagaaactggaagattttcctctttgactac

SEQ ID NO: 225 - DNA, 324 bp, synthetic construct
  source 1..324: mol_type = other DNA
gaaattgtgttgacgcagtctccaggcaccctgactttgtctccaggggaaagagccaccctctcctgcagggccagtcagagtgttggcagcaaatacttagcctggttccagcagaaacgtggccaggctcccaggctcctcatctatggtgcatccagcaggaccagtggcatccccgacaggatcagtggcagtgggtcagggacagacttcactctcaccatcagcagactggagcctgaagattttgcagtgtattactgtcagcagtatggaagttcaccctggacgttcggccaagggaccaaggtggaaatcaaa

SEQ ID NO: 226 - DNA, 21 bp, synthetic construct
  source 1..21: mol_type = other DNA
cagagtgttggcagcaaatac

SEQ ID NO: 227 - skipped

SEQ ID NO: 228 - DNA, 27 bp, synthetic construct
  source 1..27: mol_type = other DNA
cagcagtatggaagttcaccctggacg

SEQ ID NO: 229 - DNA, 1344 bp, synthetic construct
  source 1..1344: mol_type = other DNA
gaggtgcagctggtggagtctgggggaggcttggtccagccgggggggtccctgaaactctcctgtacagcctctgggttgaccctcagtgactctgctatgcactgggtccgccaggcttccgggaaagggctggagtgggttggccgtataagaaataaggctaataggtacgcgacagaatatgctgcgtcggtgaaaggcaggttcaccatttcaagagatgattcaaagaacacggcgtatctacaaatgaacagcctgaaaaccgaggacacggccgtgtattattgtactagaaactggaagattttcctctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 230 - DNA, 648 bp, synthetic construct
  source 1..648: mol_type = other DNA
gaaattgtgttgacgcagtctccaggcaccctgactttgtctccaggggaaagagccaccctctcctgcagggccagtcagagtgttggcagcaaatacttagcctggttccagcagaaacgtggccaggctcccaggctcctcatctatggtgcatccagcaggaccagtggcatccccgacaggatcagtggcagtgggtcagggacagacttcactctcaccatcagcagactggagcctgaagattttgcagtgtattactgtcagcagtatggaagttcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 231 - DNA, 375 bp, synthetic construct
  source 1..375: mol_type = other DNA
gaggtgcagctgttggagtctgggggaggcttggtacagccgggggggtccctgagactctcctgtgcagcctctggattcacctttaacaactatggcatgagctgggtccgccagggtccagggaaggggctggagtgggtctcatctattagtggtagtggtggtaccacattctacgcagactccgtgaagggccggttcaccatctccagagacaattccaagaacacgctgtatctgcaaatgaacagcctgagagccgaggacacggccgtatattactgtggcaaaggaggatattgtagtagtagcggctgccgtcactacggtatggacgtctggggccaagggaccacggtcaccgtctcctca

SEQ ID NO: 232 - DNA, 24 bp, synthetic construct
  source 1..24: mol_type = other DNA
ggattcacctttaacaactatggc

SEQ ID NO: 233 - DNA, 24 bp, synthetic construct
  source 1..24: mol_type = other DNA
attagtggtagtggtggtaccaca

SEQ ID NO: 234 - DNA, 54 bp, synthetic construct
  source 1..54: mol_type = other DNA
ggcaaaggaggatattgtagtagtagcggctgccgtcactacggtatggacgtc

SEQ ID NO: 235 - DNA, 330 bp, synthetic construct
  source 1..330: mol_type = other DNA
cagtctgtgctgactcagccaccctcagcgtctgggacccccgggcagagggtcaccatctcttgttctggaagcagctccaacatcggaaataattatatatactggtaccagcggctcccaggaacgacccccaaactcctcatctataggaataatcagcggccctcaggggtccctgaccgattctctggctccaagtctggcacctcagcctccctggccatcagtgggctccggtccgaggatgaggctgattattactgtgcagcatgggatgacaccctgagtgggtatgtcttcggaactgggaccaaggtcaccgtccta

SEQ ID NO: 236 - DNA, 24 bp, synthetic construct
  source 1..24: mol_type = other DNA
agctccaacatcggaaataattat

SEQ ID NO: 237 - skipped

SEQ ID NO: 238 - DNA, 33 bp, synthetic construct
  source 1..33: mol_type = other DNA
gcagcatgggatgacaccctgagtgggtatgtc

SEQ ID NO: 239 - DNA, 1368 bp, synthetic construct
  source 1..1368: mol_type = other DNA
gaggtgcagctgttggagtctgggggaggcttggtacagccgggggggtccctgagactctcctgtgcagcctctggattcacctttaacaactatggcatgagctgggtccgccagggtccagggaaggggctggagtgggtctcatctattagtggtagtggtggtaccacattctacgcagactccgtgaagggccggttcaccatctccagagacaattccaagaacacgctgtatctgcaaatgaacagcctgagagccgaggacacggccgtatattactgtggcaaaggaggatattgtagtagtagcggctgccgtcactacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 240 - DNA, 654 bp, synthetic construct
  source 1..654: mol_type = other DNA
cagtctgtgctgactcagccaccctcagcgtctgggacccccgggcagagggtcaccatctcttgttctggaagcagctccaacatcggaaataattatatatactggtaccagcggctcccaggaacgacccccaaactcctcatctataggaataatcagcggccctcaggggtccctgaccgattctctggctccaagtctggcacctcagcctccctggccatcagtgggctccggtccgaggatgaggctgattattactgtgcagcatgggatgacaccctgagtgggtatgtcttcggaactgggaccaaggtcaccgtcctacgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 241 - DNA, 363 bp, synthetic construct
  source 1..363: mol_type = other DNA
gaggtgcagctggtggagtctgggggaaacttggtacagcctggggggtccctgagactctcctgtgcagcctctggattcacctttaccagccatgccatgaactgggtccgccaggctccagggaaggggctggagtgggtctcagttattactggtagaggttttgacacacactacgctgactccgtgaagggccggttcaccatctccagagacatttccaaaaacacgctgtatctgcaaatgaacagcctgagagccgaggacacggccgtttattactgtgcgaaaggtctctatgattcggggaattattatatcgattactggggccagggaaccctggtcaccgtctcctca

SEQ ID NO: 242 - DNA, 24 bp, synthetic construct
  source 1..24: mol_type = other DNA
ggattcacctttaccagccatgcc

SEQ ID NO: 243 - DNA, 24 bp, synthetic construct
  source 1..24: mol_type = other DNA
attactggtagaggttttgacaca

SEQ ID NO: 244 - DNA, 42 bp, synthetic construct
  source 1..42: mol_type = other DNA
gcgaaaggtctctatgattcggggaattattatatcgattac

SEQ ID NO: 245 - DNA, 333 bp, synthetic construct
  source 1..333: mol_type = other DNA
cagtctgtgttgacgcagccgccctcagtgtctgcggccccaggacagaaggtcaccatctcctgctctggaagcagctccaacattgggaataattatgtttcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatctcagcctgagtttcaattgggtgttcggcggagggaccaagctgaccgtccta

SEQ ID NO: 246 - DNA, 24 bp, synthetic construct
  source 1..24: mol_type = other DNA
agctccaacattgggaataattat

SEQ ID NO: 247 - skipped

SEQ ID NO: 248 - DNA, 36 bp, synthetic construct
  source 1..36: mol_type = other DNA
ggaacatgggatctcagcctgagtttcaattgggtg

SEQ ID NO: 249 - DNA, 1347 bp, synthetic construct
  source 1..1347: mol_type = other DNA
gaggtgcagctggtggagtctgggggaaacttggtacagcctggggggtccctgagactctcctgtgcagcctctggattcacctttaccagccatgccatgaactgggtccgccaggctccagggaaggggctggagtgggtctcagttattactggtagaggttttgacacacactacgctgactccgtgaagggccggttcaccatctccagagacatttccaaaaacacgctgtatctgcaaatgaacagcctgagagccgaggacacggccgtttattactgtgcgaaaggtctctatgattcggggaattattatatcgattactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 250 - DNA, 654 bp, synthetic construct
  source 1..654: mol_type = other DNA
cagtctgtgttgacgcagccgccctcagtgtctgcggccccaggacagaaggtcaccatctcctgctctggaagcagctccaacattgggaataattatgtttcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatctcagcctgagtttcaattgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 251 - DNA, 357 bp, synthetic construct
  source 1..357: mol_type = other DNA
caggtgcagctacagcagtggggcgcaggactgttgaagccttcggcgaccctgtcccgcacctgcgctgtctatggtgggtccttcagtggttactactggaactggatccgccagtccccagggaaggggctggaatggattggggaaatccttcatagtggaagaaccaactacaacccgtccctcaagagtcgagtcaccatatcagtagacacgtccaagaaccagttctccctgaagctgacctctgtgaccgccgcggacacggctgtatattactgtgcgggaaggatagcagctcgtcacggctggttcgacccctggggccagggaaccctggtcaccgtctcctca

SEQ ID NO: 252 - DNA, 24 bp, synthetic construct
  source 1..24: mol_type = other DNA
ggtgggtccttcagtggttactac

SEQ ID NO: 253 - DNA, 21 bp, synthetic construct
  source 1..21: mol_type = other DNA
atccttcatagtggaagaacc

SEQ ID NO: 254 - DNA, 39 bp, synthetic construct
  source 1..39: mol_type = other DNA
gcgggaaggatagcagctcgtcacggctggttcgacccc

SEQ ID NO: 255 - DNA, 321 bp, synthetic construct
  source 1..321: mol_type = other DNA
gacatccagatgacccagtctccatcttccgtgtctacatctgtaggagacagagtcaccatctcttgtcgggcgagtcaggatattcgcaagtggttagcctggtatcaacagaaaccaggaaaagcccctaaactcctgatctatgctacatccagtttgcaaagtggggtcccttcaaggttcagcggcagtggatctgggacagatttcactctcaccatcagcagcctgcagcctgaggattttgcaacttacttttgtcaacaggctaacagtttcccgttcacttttggccaggggaccaagctggagatcaaa

SEQ ID NO: 256 - DNA, 18 bp, synthetic construct
  source 1..18: mol_type = other DNA
caggatattcgcaagtgg

SEQ ID NO: 257 - skipped

SEQ ID NO: 258 - DNA, 27 bp, synthetic construct
  source 1..27: mol_type = other DNA
caacaggctaacagtttcccgttcact

SEQ ID NO: 259 - DNA, 1341 bp, synthetic construct
  source 1..1341: mol_type = other DNA
caggtgcagctacagcagtggggcgcaggactgttgaagccttcggcgaccctgtcccgcacctgcgctgtctatggtgggtccttcagtggttactactggaactggatccgccagtccccagggaaggggctggaatggattggggaaatccttcatagtggaagaaccaactacaacccgtccctcaagagtcgagtcaccatatcagtagacacgtccaagaaccagttctccctgaagctgacctctgtgaccgccgcggacacggctgtatattactgtgcgggaaggatagcagctcgtcacggctggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 260 - DNA, 645 bp, synthetic construct
  source 1..645: mol_type = other DNA
gacatccagatgacccagtctccatcttccgtgtctacatctgtaggagacagagtcaccatctcttgtcgggcgagtcaggatattcgcaagtggttagcctggtatcaacagaaaccaggaaaagcccctaaactcctgatctatgctacatccagtttgcaaagtggggtcccttcaaggttcagcggcagtggatctgggacagatttcactctcaccatcagcagcctgcagcctgaggattttgcaacttacttttgtcaacaggctaacagtttcccgttcacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 261 - DNA, 348 bp, synthetic construct
  source 1..348: mol_type = other DNA
caggtgcagctgcaggagtcgggcccaggactggtgaagccttcggagaccctgtccctcacctgcactgtctctggtgactccatcaataattactactggacctggctccggcagcccccagggaagggactggagtggattggttatatctattacagtgggagcgccaactacaacccctccctcaagagtcgagtcaccatatcagtagacacgtccaagaaccagttctccctgaagctaaattctgtgaccgctgcggacacggccgtgtattactgtgcgagaggggcggtcaagtacttccggcattggggccagggcaccctggtcaccgtctcctca

SEQ ID NO: 262 - DNA, 24 bp, synthetic construct
  source 1..24: mol_type = other DNA
ggtgactccatcaataattactac

SEQ ID NO: 263 - DNA, 21 bp, synthetic construct
  source 1..21: mol_type = other DNA
atctattacagtgggagcgcc

SEQ ID NO: 264 - DNA, 30 bp, synthetic construct
  source 1..30: mol_type = other DNA
gcgagaggggcggtcaagtacttccggcat

SEQ ID NO: 265 - DNA, 324 bp, synthetic construct
  source 1..324: mol_type = other DNA
gaaattgtgttgacgcagtctccgggcaccctctctttgtctccaggggaaagagccaccctctcctgcagggccagtcagactattaaccacaacaacttagcctggtaccagcagagacctggccaggctcccaggctcctcatctatggtgcatccaacagggccactgccatcccagacaggttcagtggcagtgggtctgggacagacttcactctcaccatcagcagactggagcctgaagattttgaagtgtattcttgtcagcagtatggtagcttgccgctcactttcggcggagggaccaaggtggagatcaaa

SEQ ID NO: 266 - DNA, 21 bp, synthetic construct
  source 1..21: mol_type = other DNA
cagactattaaccacaacaac

SEQ ID NO: 267 - skipped

SEQ ID NO: 268 - DNA, 27 bp, synthetic construct
  source 1..27: mol_type = other DNA
cagcagtatggtagcttgccgctcact

SEQ ID NO: 269 - DNA, 1332 bp, synthetic construct
  source 1..1332: mol_type = other DNA
caggtgcagctgcaggagtcgggcccaggactggtgaagccttcggagaccctgtccctcacctgcactgtctctggtgactccatcaataattactactggacctggctccggcagcccccagggaagggactggagtggattggttatatctattacagtgggagcgccaactacaacccctccctcaagagtcgagtcaccatatcagtagacacgtccaagaaccagttctccctgaagctaaattctgtgaccgctgcggacacggccgtgtattactgtgcgagaggggcggtcaagtacttccggcattggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 270 - DNA, 648 bp, synthetic construct
  source 1..648: mol_type = other DNA
gaaattgtgttgacgcagtctccgggcaccctctctttgtctccaggggaaagagccaccctctcctgcagggccagtcagactattaaccacaacaacttagcctggtaccagcagagacctggccaggctcccaggctcctcatctatggtgcatccaacagggccactgccatcccagacaggttcagtggcagtgggtctgggacagacttcactctcaccatcagcagactggagcctgaagattttgaagtgtattcttgtcagcagtatggtagcttgccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 271 - DNA, 357 bp, synthetic construct
  source 1..357: mol_type = other DNA
caggtgcagctggtggagtctgggggaggcgtggtccagcctgggaggtccctgagactctcctgtgcagcgtctggattcaccttcagtacatatggcatgcactgggtccgccaggctccaggcaaggggctggagtgggtggcagttatttggcatgatggaagtgataaatattatgtagactccgtgaagggccgattctccatcgccagagacaattccaagaacacgctttatctgcaaatgaatagtctgagagtcgaggacacgggtatatattactgtgcgagaaggggtatacgtggaaccgtttttgaccactggggcctgggaaccctggtcaccgtctcctca

SEQ ID NO: 272 - DNA, 24 bp, synthetic construct
  source 1..24: mol_type = other DNA
ggattcaccttcagtacatatggc

SEQ ID NO: 273 - DNA, 24 bp, synthetic construct
  source 1..24: mol_type = other DNA
atttggcatgatggaagtgataaa

SEQ ID NO: 274 - DNA, 36 bp, synthetic construct
  source 1..36: mol_type = other DNA
gcgagaaggggtatacgtggaaccgtttttgaccac

SEQ ID NO: 275 - DNA, 321 bp, synthetic construct
  source 1..321: mol_type = other DNA
gacatccagatgacccagtctccttccaccctgtctgcatctgtaggagacagagtcaccctcacttgtcgggccagtcagagtattagtaacaagttggcctggtatcagcagaaaccagggaaagcccctaacctcctgatctataaggcgtctaatttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaa

SEQ ID NO: 276 - DNA, 18 bp, synthetic construct
  source 1..18: mol_type = other DNA
cagagtattagtaacaag

SEQ ID NO: 277 - skipped

SEQ ID NO: 278 - DNA, 27 bp, synthetic construct
  source 1..27: mol_type = other DNA
caacagtataatagttattcgtggacg

SEQ ID NO: 279 - DNA, 1341 bp, synthetic construct
  source 1..1341: mol_type = other DNA
caggtgcagctggtggagtctgggggaggcgtggtccagcctgggaggtccctgagactctcctgtgcagcgtctggattcaccttcagtacatatggcatgcactgggtccgccaggctccaggcaaggggctggagtgggtggcagttatttggcatgatggaagtgataaatattatgtagactccgtgaagggccgattctccatcgccagagacaattccaagaacacgctttatctgcaaatgaatagtctgagagtcgaggacacgggtatatattactgtgcgagaaggggtatacgtggaaccgtttttgaccactggggcctgggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 280 - DNA, 645 bp, synthetic construct
  source 1..645: mol_type = other DNA
gacatccagatgacccagtctccttccaccctgtctgcatctgtaggagacagagtcaccctcacttgtcgggccagtcagagtattagtaacaagttggcctggtatcagcagaaaccagggaaagcccctaacctcctgatctataaggcgtctaatttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 281 - DNA, 363 bp, synthetic construct
  source 1..363: mol_type = other DNA
caggtgcagctggtggagtctgggggaggcgtggtccagcctgggacgtccctgagactctcctgtgcagcgtcaggattcaccttcagtagctatggcatgcactgggtccgccaggctccaggcaaggggctggagtgggtggcagttatatggattgatggaagtaataaatattatgcagactccgtgaagggccgattcaccatctccagagacaattccaagaacacgctgtatctgcaaatgaacagcctgagagccgaggacacggctgtgtattactgtgcgagaagggggggtatagtagtagctgccccctttgactactggggccagggaaccctggtcaccgtctcctca

SEQ ID NO: 282 - DNA, 24 bp, synthetic construct
  source 1..24: mol_type = other DNA
ggattcaccttcagtagctatggc

SEQ ID NO: 283 - DNA, 24 bp, synthetic construct
  source 1..24: mol_type = other DNA
atatggattgatggaagtaataaa

SEQ ID NO: 284 - DNA, 42 bp, synthetic construct
  source 1..42: mol_type = other DNA
gcgagaagggggggtatagtagtagctgccccctttgactac

SEQ ID NO: 285 - DNA, 324 bp, synthetic construct; identical to SEQ ID NO: 185
  source 1..324: mol_type = other DNA

SEQ ID NO: 286 - DNA, 18 bp, synthetic construct
  source 1..18: mol_type = other DNA
cagagcattagcagctat

SEQ ID NO: 287 - skipped

SEQ ID NO: 288 - DNA, 30 bp, synthetic construct; identical to SEQ ID NO: 188
  source 1..30: mol_type = other DNA

SEQ ID NO: 289 - DNA, 1347 bp, synthetic construct
  source 1..1347: mol_type = other DNA
caggtgcagctggtggagtctgggggaggcgtggtccagcctgggacgtccctgagactctcctgtgcagcgtcaggattcaccttcagtagctatggcatgcactgggtccgccaggctccaggcaaggggctggagtgggtggcagttatatggattgatggaagtaataaatattatgcagactccgtgaagggccgattcaccatctccagagacaattccaagaacacgctgtatctgcaaatgaacagcctgagagccgaggacacggctgtgtattactgtgcgagaagggggggtatagtagtagctgccccctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 290 - DNA, 648 bp, synthetic construct; identical to SEQ ID NO: 190
  source 1..648: mol_type = other DNA

SEQ ID NO: 291 - DNA, 378 bp, synthetic construct; identical to SEQ ID NO: 211
  source 1..378: mol_type = other DNA

SEQ ID NO: 292 - DNA, 24 bp, synthetic construct
  source 1..24: mol_type = other DNA
ggttacgccttcaccacctatggt

SEQ ID NO: 293 - DNA, 24 bp, synthetic construct
  source 1..24: mol_type = other DNA
atcagcgcttacaatggaaataca

SEQ ID NO: 294 - DNA, 57 bp, synthetic construct; identical to SEQ ID NO: 214
  source 1..57: mol_type = other DNA

SEQ ID NO: 295 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgctttgtatttctgtcagcagtattatggctcaccttggacgttcggccaagggaccaaggtggaaatcaaa

SEQ ID NO: 296 - DNA, 21 bp, synthetic construct
  source 1..21: mol_type = other DNA
cagagtgttagcagcagctac

SEQ ID NO: 297 - skipped

SEQ ID NO: 298 - DNA, 27 bp, synthetic construct
  source 1..27: mol_type = other DNA
cagcagtattatggctcaccttggacg

SEQ ID NO: 299 - DNA, 1362 bp, synthetic construct
  source 1..1362: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 300 - DNA, 648 bp, synthetic construct
  source 1..648: mol_type = other DNA
gaaattgtgttgacgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgctttgtatttctgtcagcagtattatggctcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 301 - DNA, 357 bp, synthetic construct; identical to SEQ ID NO: 251
  source 1..357: mol_type = other DNA

SEQ ID NO: 302 - DNA, 24 bp, synthetic construct
  source 1..24: mol_type = other DNA
ggtgggtccttcagtggttactac

SEQ ID NO: 303 - DNA, 21 bp, synthetic construct
  source 1..21: mol_type = other DNA
atccttcatagtggaagaacc

SEQ ID NO: 304 - DNA, 39 bp, synthetic construct; identical to SEQ ID NO: 254
  source 1..39: mol_type = other DNA

SEQ ID NO: 305 - DNA, 321 bp, synthetic construct; identical to SEQ ID NO: 255
  source 1..321: mol_type = other DNA

SEQ ID NO: 306 - DNA, 18 bp, synthetic construct
  source 1..18: mol_type = other DNA
caggatattcgcaagtgg

SEQ ID NO: 307 - skipped

SEQ ID NO: 308 - DNA, 27 bp, synthetic construct
  source 1..27: mol_type = other DNA
caacaggctaacagtttcccgttcact

SEQ ID NO: 309 - DNA, 1350 bp, synthetic construct
  source 1..1350: mol_type = other DNA
caggtgcagctacagcagtggggcgcaggactgttgaagccttcggcgaccctgtcccgcacctgcgctgtctatggtgggtccttcagtggttactactggaactggatccgccagtccccagggaaggggctggaatggattggggaaatccttcatagtggaagaaccaactacaacccgtccctcaagagtcgagtcaccatatcagtagacacgtccaagaaccagttctccctgaagctgacctctgtgaccgccgcggacacggctgtatattactgtgcgggaaggatagcagctcgtcacggctggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 310 - DNA, 645 bp, synthetic construct; identical to SEQ ID NO: 260
  source 1..645: mol_type = other DNA

SEQ ID NO: 311 - DNA, 348 bp, synthetic construct; identical to SEQ ID NO: 261
  source 1..348: mol_type = other DNA

SEQ ID NO: 312 - DNA, 24 bp, synthetic construct
  source 1..24: mol_type = other DNA
ggtgactccatcaataattactac

SEQ ID NO: 313 - DNA, 21 bp, synthetic construct
  source 1..21: mol_type = other DNA
atctattacagtgggagcgcc

SEQ ID NO: 314 - DNA, 30 bp, synthetic construct; identical to SEQ ID NO: 264
  source 1..30: mol_type = other DNA

SEQ ID NO: 315 - DNA, 324 bp, synthetic construct; identical to SEQ ID NO: 265
  source 1..324: mol_type = other DNA

SEQ ID NO: 316 - DNA, 21 bp, synthetic construct
  source 1..21: mol_type = other DNA
cagactattaaccacaacaac

SEQ ID NO: 317 - skipped

SEQ ID NO: 318 - DNA, 27 bp, synthetic construct
  source 1..27: mol_type = other DNA
cagcagtatggtagcttgccgctcact

SEQ ID NO: 319 - DNA, 1341 bp, synthetic construct
  source 1..1341: mol_type = other DNA
caggtgcagctgcaggagtcgggcccaggactggtgaagccttcggagaccctgtccctcacctgcactgtctctggtgactccatcaataattactactggacctggctccggcagcccccagggaagggactggagtggattggttatatctattacagtgggagcgccaactacaacccctccctcaagagtcgagtcaccatatcagtagacacgtccaagaaccagttctccctgaagctaaattctgtgaccgctgcggacacggccgtgtattactgtgcgagaggggcggtcaagtacttccggcattggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 320 - DNA, 648 bp, synthetic construct; identical to SEQ ID NO: 270
  source 1..648: mol_type = other DNA

SEQ ID NO: 321 - DNA, 357 bp, synthetic construct; identical to SEQ ID NO: 271
  source 1..357: mol_type = other DNA

SEQ ID NO: 322 - DNA, 24 bp, synthetic construct
  source 1..24: mol_type = other DNA
ggattcaccttcagtacatatggc

SEQ ID NO: 323 - DNA, 24 bp, synthetic construct
  source 1..24: mol_type = other DNA
atttggcatgatggaagtgataaa

SEQ ID NO: 324 - DNA, 36 bp, synthetic construct; identical to SEQ ID NO: 274
  source 1..36: mol_type = other DNA

SEQ ID NO: 325 - DNA, 321 bp, synthetic construct; identical to SEQ ID NO: 275
  source 1..321: mol_type = other DNA

SEQ ID NO: 326 - DNA, 18 bp, synthetic construct
  source 1..18: mol_type = other DNA
cagagtattagtaacaag

SEQ ID NO: 327 - skipped

SEQ ID NO: 328 - DNA, 27 bp, synthetic construct
  source 1..27: mol_type = other DNA
caacagtataatagttattcgtggacg

SEQ ID NO: 329 - DNA, 1350 bp, synthetic construct
  source 1..1350: mol_type = other DNA
caggtgcagctggtggagtctgggggaggcgtggtccagcctgggaggtccctgagactctcctgtgcagcgtctggattcaccttcagtacatatggcatgcactgggtccgccaggctccaggcaaggggctggagtgggtggcagttatttggcatgatggaagtgataaatattatgtagactccgtgaagggccgattctccatcgccagagacaattccaagaacacgctttatctgcaaatgaatagtctgagagtcgaggacacgggtatatattactgtgcgagaaggggtatacgtggaaccgtttttgaccactggggcctgggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 330 - DNA, 645 bp, synthetic construct; identical to SEQ ID NO: 280
  source 1..645: mol_type = other DNA

SEQ ID NO: 331 - DNA, 363 bp, synthetic construct; identical to SEQ ID NO: 241
  source 1..363: mol_type = other DNA

SEQ ID NO: 332 - DNA, 24 bp, synthetic construct
  source 1..24: mol_type = other DNA
ggattcacctttaccagccatgcc

SEQ ID NO: 333 - DNA, 24 bp, synthetic construct
  source 1..24: mol_type = other DNA
attactggtagaggttttgacaca

SEQ ID NO: 334 - DNA, 42 bp, synthetic construct; identical to SEQ ID NO: 244
  source 1..42: mol_type = other DNA

SEQ ID NO: 335 - DNA, 333 bp, synthetic construct; identical to SEQ ID NO: 245
  source 1..333: mol_type = other DNA

SEQ ID NO: 336 - DNA, 24 bp, synthetic construct
  source 1..24: mol_type = other DNA
agctccaacattgggaataattat

SEQ ID NO: 337 - skipped

SEQ ID NO: 338 - DNA, 36 bp, synthetic construct; identical to SEQ ID NO: 248
  source 1..36: mol_type = other DNA

SEQ ID NO: 339 - DNA, 1356 bp, synthetic construct
  source 1..1356: mol_type = other DNA
gaggtgcagctggtggagtctgggggaaacttggtacagcctggggggtccctgagactctcctgtgcagcctctggattcacctttaccagccatgccatgaactgggtccgccaggctccagggaaggggctggagtgggtctcagttattactggtagaggttttgacacacactacgctgactccgtgaagggccggttcaccatctccagagacatttccaaaaacacgctgtatctgcaaatgaacagcctgagagccgaggacacggccgtttattactgtgcgaaaggtctctatgattcggggaattattatatcgattactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 340 - DNA, 654 bp, synthetic construct; identical to SEQ ID NO: 250
  source 1..654: mol_type = other DNA

SEQ ID NO: 341 - DNA, 363 bp, synthetic construct; identical to SEQ ID NO: 281
  source 1..363: mol_type = other DNA

SEQ ID NO: 342 - DNA, 24 bp, synthetic construct
  source 1..24: mol_type = other DNA
ggattcaccttcagtagctatggc

SEQ ID NO: 343 - DNA, 24 bp, synthetic construct
  source 1..24: mol_type = other DNA
atatggattgatggaagtaataaa

SEQ ID NO: 344 - DNA, 42 bp, synthetic construct; identical to SEQ ID NO: 284
  source 1..42: mol_type = other DNA

SEQ ID NO: 345 - DNA, 324 bp, synthetic construct; identical to SEQ ID NO: 185
  source 1..324: mol_type = other DNA

SEQ ID NO: 346 - DNA, 18 bp, synthetic construct
  source 1..18: mol_type = other DNA
cagagcattagcagctat

SEQ ID NO: 347 - skipped

SEQ ID NO: 348 - DNA, 30 bp, synthetic construct; identical to SEQ ID NO: 188
  source 1..30: mol_type = other DNA

SEQ ID NO: 349 - DNA, 1356 bp, synthetic construct
  source 1..1356: mol_type = other DNA
caggtgcagctggtggagtctgggggaggcgtggtccagcctgggacgtccctgagactctcctgtgcagcgtcaggattcaccttcagtagctatggcatgcactgggtccgccaggctccaggcaaggggctggagtgggtggcagttatatggattgatggaagtaataaatattatgcagactccgtgaagggccgattcaccatctccagagacaattccaagaacacgctgtatctgcaaatgaacagcctgagagccgaggacacggctgtgtattactgtgcgagaagggggggtatagtagtagctgccccctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 350 - DNA, 648 bp, synthetic construct; identical to SEQ ID NO: 190
  source 1..648: mol_type = other DNA

SEQ ID NO: 351 - DNA, 357 bp, synthetic construct; identical to SEQ ID NO: 181
  source 1..357: mol_type = other DNA

SEQ ID NO: 352 - DNA, 24 bp, synthetic construct
  source 1..24: mol_type = other DNA
ggaatcaccttcagaaattatggc

SEQ ID NO: 353 - DNA, 24 bp, synthetic construct
  source 1..24: mol_type = other DNA
atgtggtatgatggaagtaataag

SEQ ID NO: 354 - DNA, 42 bp, synthetic construct; identical to SEQ ID NO: 184
  source 1..42: mol_type = other DNA

SEQ ID NO: 355 - DNA, 324 bp, synthetic construct; identical to SEQ ID NO: 185
  source 1..324: mol_type = other DNA

SEQ ID NO: 356 - DNA, 18 bp, synthetic construct
  source 1..18: mol_type = other DNA
cagagcattagcagctat

SEQ ID NO: 357 - skipped

SEQ ID NO: 358 - DNA, 30 bp, synthetic construct; identical to SEQ ID NO: 188
  source 1..30: mol_type = other DNA

SEQ ID NO: 359 - DNA, 1350 bp, synthetic construct
  source 1..1350: mol_type = other DNA
caggtgcagctggtggagtctgggggaggcgtggtccagcctgggaggtccctgagactctcctgtacagcgtctggaatcaccttcagaaattatggcatgcactgggtccgccaggctccaggcaaggggctggagtgggtggcagttatgtggtatgatggaagtaataagtactatgcagactccgtgaagggccgtttcaccatctccggagacaattccaaggtgtatctgcaaatgaacagcctgagagccgaggacacggctgtatattactgtgcgagaaggggcactataagaacagctgccccttttgactactggggtcagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 360 - DNA, 648 bp, synthetic construct; identical to SEQ ID NO: 190
  source 1..648: mol_type = other DNA

SEQ ID NO: 361 - DNA, 666 bp, Homo sapiens
  source 1..666: mol_type = genomic DNA
atgtcacagaccaaaatgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcac

SEQ ID NO: 362 - protein, 222 aa, Homo sapiens
MSQTKMLKVRVTLFCILAGIVLAMTAVVTDHWAVLSPHMEHHNTTCEAAHFGLWRICTKRIPMDDSKTCGPITLPGEKNCSYFRHFNPGESSEIFEFTTQKEYSISAAAIAIFSLGFIILGSLCVLLSLGKKRDYLLRPASMFYAFAGLCILVSVEVMRQSVKRMIDSEDTVWIEYYYSWSFACACAAFILLFLGGLALLLFSLPRMPRNPWESCMDAEPEH

SEQ ID NO: 363 - protein, 29 aa, Mus musculus
MHRPRRRGTRPPPLALLAALLLAARGADA

SEQ ID NO: 364 - protein, 6 aa, synthetic construct
LLQGSG

SEQ ID NO: 365 - protein, 6 aa, synthetic construct
HHHHHH

SEQ ID NO: 366 - RNA, 16 nt, synthetic construct
  source 1..16: mol_type = other RNA
gttttagagctatgct

SEQ ID NO: 367 - RNA, 22 nt, synthetic construct
  source 1..22: mol_type = other RNA
gttttagagctatgctgttttg

SEQ ID NO: 368 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 369 - RNA, 72 nt, synthetic construct
  source 1..72: mol_type = other RNA
aaacagcatagcaagttaaaataaggctagtccgttatcaacttgaaaaagtggcaccgagtcggtgctttt

SEQ ID NO: 370 - RNA, 82 nt, synthetic construct
  source 1..82: mol_type = other RNA
gttggaaccattcaaaacagcatagcaagttaaaataaggctagtccgttatcaacttgaaaaagtggcaccgagtcggtgc

SEQ ID NO: 371 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 372 - RNA, 82 nt, synthetic construct; identical to SEQ ID NO: 370
  source 1..82: mol_type = other RNA

SEQ ID NO: 373 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 374 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 375 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 376 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 377 - RNA, 92 nt, synthetic construct
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 378 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 379 - RNA, 100 nt, synthetic construct
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = a, c, u, g, unknown or other
  misc_difference 5: note = a, c, u, g, unknown or other
  misc_difference 6: note = a, c, u, g, unknown or other
  misc_difference 7: note = a, c, u, g, unknown or other
  misc_difference 8: note = a, c, u, g, unknown or other
  misc_difference 9: note = a, c, u, g, unknown or other
  misc_difference 10: note = a, c, u, g, unknown or other
  misc_difference 11: note = a, c, u, g, unknown or other
  misc_difference 12: note = a, c, u, g, unknown or other
  misc_difference 13: note = a, c, u, g, unknown or other
  misc_difference 14: note = a, c, u, g, unknown or other
  misc_difference 15: note = a, c, u, g, unknown or other
  misc_difference 16: note = a, c, u, g, unknown or other
  misc_difference 17: note = a, c, u, g, unknown or other
  misc_difference 18: note = a, c, u, g, unknown or other
  misc_difference 19: note = a, c, u, g, unknown or other
  misc_difference 20: note = a, c, u, g, unknown or other
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  modified_base 37: mod_base = OTHER; note = 2'-O-methyl modified nucleotide
  modified_base 38: mod_base = OTHER; note = 2'-O-methyl modified nucleotide
  modified_base 39: mod_base = OTHER; note = 2'-O-methyl modified nucleotide
  modified_base 40: mod_base = OTHER; note = 2'-O-methyl modified nucleotide
  modified_base 69: mod_base = OTHER; note = 2'-O-methyl modified nucleotide
  modified_base 70: mod_base = OTHER; note = 2'-O-methyl modified nucleotide
  modified_base 71: mod_base = OTHER; note = 2'-O-methyl modified nucleotide
  modified_base 72: mod_base = OTHER; note = 2'-O-methyl modified nucleotide
  modified_base 73: mod_base = OTHER; note = 2'-O-methyl modified nucleotide
  modified_base 74: mod_base = OTHER; note = 2'-O-methyl modified nucleotide
  modified_base 75: mod_base = OTHER; note = 2'-O-methyl modified nucleotide
  modified_base 76: mod_base = OTHER; note = 2'-O-methyl modified nucleotide
  modified_base 77: mod_base = OTHER; note = 2'-O-methyl modified nucleotide
  modified_base 78: mod_base = OTHER; note = 2'-O-methyl modified nucleotide
  modified_base 79: mod_base = OTHER; note = 2'-O-methyl modified nucleotide
  modified_base 80: mod_base = OTHER; note = 2'-O-methyl modified nucleotide
  modified_base 81: mod_base = OTHER; note = 2'-O-methyl modified nucleotide
  modified_base 82: mod_base = OTHER; note = 2'-O-methyl modified nucleotide
  modified_base 83: mod_base = OTHER; note = 2'-O-methyl modified nucleotide
  modified_base 84: mod_base = OTHER; note = 2'-O-methyl modified nucleotide
  modified_base 85: mod_base = OTHER; note = 2'-O-methyl modified nucleotide
  modified_base 86: mod_base = OTHER; note = 2'-O-methyl modified nucleotide
  modified_base 87: mod_base = OTHER; note = 2'-O-methyl modified nucleotide
  modified_base 88: mod_base = OTHER; note = 2'-O-methyl modified nucleotide
  modified_base 89: mod_base = OTHER; note = 2'-O-methyl modified nucleotide
  modified_base 90: mod_base = OTHER; note = 2'-O-methyl modified nucleotide
  modified_base 91: mod_base = OTHER; note = 2'-O-methyl modified nucleotide
  modified_base 92: mod_base = OTHER; note = 2'-O-methyl modified nucleotide
  modified_base 93: mod_base = OTHER; note = 2'-O-methyl modified nucleotide
  modified_base 94: mod_base = OTHER; note = 2'-O-methyl modified nucleotide
  modified_base 95: mod_base = OTHER; note = 2'-O-methyl modified nucleotide
  modified_base 96: mod_base = OTHER; note = 2'-O-methyl modified nucleotide
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
nnnnnnnnnnnnnnnnnnnngttttagagctagaaatagcaagttaaaataaggctagtccgttatcaacttgaaaaagtggcaccgagtcggtgctttt

SEQ ID NO: 380 - protein, 28 aa, synthetic construct
EQKLISEEDLGGEQKLISEEDLHHHHHH

SEQ ID NO: 381 - protein, 12 aa, synthetic construct
GSGEGEGSEGSG

SEQ ID NO: 382 - protein, 12 aa, synthetic construct
GGSEGEGSEGGS

SEQ ID NO: 383 - protein, 5 aa, synthetic construct
GGGGS

SEQ ID NO: 384 - protein, 4 aa, synthetic construct
GGGS

SEQ ID NO: 385 - protein, 7 aa, synthetic construct
PKKKRKV

SEQ ID NO: 386 - protein, 7 aa, synthetic construct
PKKKRRV

SEQ ID NO: 387 - protein, 16 aa, synthetic construct
KRPAATKKAGQAKKKK

SEQ ID NO: 388 - skipped

SEQ ID NO: 389 - protein, 5 aa, synthetic construct
LLQGG

SEQ ID NO: 390 - protein, 4 aa, synthetic construct
LLQG

SEQ ID NO: 391 - protein, 6 aa, synthetic construct
LSLSQG

SEQ ID NO: 392 - protein, 8 aa, synthetic construct
GGGLLQGG

SEQ ID NO: 393 - protein, 5 aa, synthetic construct
GLLQG

SEQ ID NO: 394 - skipped

SEQ ID NO: 395 - protein, 10 aa, synthetic construct
GSPLAQSHGG

SEQ ID NO: 396 - protein, 7 aa, synthetic construct
GLLQGGG

SEQ ID NO: 397 - protein, 6 aa, synthetic construct
GLLQGG

SEQ ID NO: 398 - protein, 4 aa, synthetic construct
GLLQ

SEQ ID NO: 399 - protein, 8 aa, synthetic construct
LLQLLQGA

SEQ ID NO: 400 - protein, 5 aa, synthetic construct
LLQGA

SEQ ID NO: 401 - protein, 7 aa, synthetic construct
LLQYQGA

SEQ ID NO: 402 - protein, 6 aa, synthetic construct
LLQGSG

SEQ ID NO: 403 - protein, 6 aa, synthetic construct
LLQYQG

SEQ ID NO: 404 - protein, 7 aa, synthetic construct
LLQLLQG

SEQ ID NO: 405 - protein, 5 aa, synthetic construct
SLLQG

SEQ ID NO: 406 - protein, 5 aa, synthetic construct
LLQLQ

SEQ ID NO: 407 - protein, 6 aa, synthetic construct
LLQLLQ

SEQ ID NO: 408 - protein, 5 aa, synthetic construct
LLQGR

SEQ ID NO: 409 - protein, 12 aa, synthetic construct
EQKLISEEDLGG

SEQ ID NO: 410 - protein, 233 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 411 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 412-415 - skipped (4 entries)

SEQ ID NO: 416 - RNA, 19 nt, synthetic construct
  source 1..19: mol_type = other RNA
cctcggtatttattgtcta

SEQ ID NO: 417 - RNA, 19 nt, synthetic construct
  source 1..19: mol_type = other RNA
tagacaataaataccgagg

SEQ ID NO: 418 - DNA, 18 bp, synthetic construct
  source 1..18: mol_type = other DNA
aaggccgtggtgctgatg

SEQ ID NO: 419 - DNA, 21 bp, synthetic construct
  source 1..21: mol_type = other DNA
tctccagagctgggttgttct

SEQ ID NO: 420 - DNA, 23 bp, synthetic construct
  source 1..23: mol_type = other DNA
aagaacaccatgatgcgcaaggc

SEQ ID NO: 421 - RNA, 23 nt, synthetic construct
  source 1..23: mol_type = other RNA
  modified_base 1: mod_base = OTHER; note = Inverted abasic site with phosphorothioate linkage attached to 2,3,5,6-tetrafluorophenyl 26-(2,5-dioxo-2,5-di hydro-1H-pyrrol-1-yl)-18-(3-(2,5-dioxo-2,5-dihydro-1H-pyrr ol-1-yl)propanamido)-17,24-dioxo-4,7,10,13-tetraoxa-16,23- diazahexacosanoate
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = Inverted abasic site
ncctcggtatttattgtctattn

SEQ ID NO: 422 - RNA, 21 nt, synthetic construct
  source 1..21: mol_type = other RNA
  modified_base 1: mod_base = OTHER; note = Vinylphosphate uridine with phosphorothioate linkage
  modified_base 2: mod_base = OTHER; note = 2'-fluoro modified nucleotide with phosphorothioate linkage
  modified_base 3: mod_base = OTHER; note = 2'-fluoro modified nucleotide with phosphorothioate linkage
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = Inverted abasic site with phosphorothioate linkage
  modified_base 21: mod_base = OTHER; note = Inverted abasic site
tagacaataaataccgaggnn

SEQ ID NO: 423 - protein, 15 aa, synthetic construct
GGGGSGGGGSGGGGS

SEQ ID NO: 424 - RNA, 100 nt, synthetic construct
  source 1..100: mol_type = other RNA
  misc_difference 1..100: note = This sequence may encompass 95-100 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 425 - protein, 4 aa, synthetic construct
GGFG

SEQ ID NO: 426 - protein, 4 aa, synthetic construct
ALAL

SEQ ID NO: 427 - protein, 4 aa, synthetic construct
GFLG

SEQ ID NO: 428 - DNA, 360 bp, synthetic construct
  source 1..360: mol_type = other DNA
caggtgcagctggtggagtctgggggaggcgtggtccagcctgggaggtccctgagactctcctgtgcagcctctggattcacattcagtacctatggcatgtactgggtccgccagactccaggcaaggggctggagtgggtgacagttatatcatttgatggaaataaaaaatactatgcagactccgtgaagggccgattcaccatctccagagacaattccaaaaacacgctgtttctgcaaatgaacagcctgaaaactgaggacacggctgtatattactgtgcgaaatcttctaactggaactacggttcttttgatatatggggccaagggacaatggtcaccgtctcttca

SEQ ID NO: 429 - protein, 120 aa, synthetic construct
QVQLVESGGGVVQPGRSLRLSCAASGFTFSTYGMYWVRQTPGKGLEWVTVISFDGNKKYYADSVKGRFTISRDNSKNTLFLQMNSLKTEDTAVYYCAKSSNWNYGSFDIWGQGTMVTVSS

SEQ ID NO: 430 - DNA, 24 bp, synthetic construct
  source 1..24: mol_type = other DNA
ggattcacattcagtacctatggc

SEQ ID NO: 431 - protein, 8 aa, synthetic construct
GFTFSTYG

SEQ ID NO: 432 - DNA, 24 bp, synthetic construct
  source 1..24: mol_type = other DNA
atatcatttgatggaaataaaaaa

SEQ ID NO: 433 - protein, 8 aa, synthetic construct
ISFDGNKK

SEQ ID NO: 434 - DNA, 39 bp, synthetic construct
  source 1..39: mol_type = other DNA
gcgaaatcttctaactggaactacggttcttttgatata

SEQ ID NO: 435 - protein, 13 aa, synthetic construct
AKSSNWNYGSFDI

SEQ ID NO: 436 - DNA, 333 bp, synthetic construct
  source 1..333: mol_type = other DNA
cagtctgtgctgacgcagccgccctcagtgtctggggccccagggcagagggtcaccatctcctgcactgggagcagctccaacatcggggcaggttatgatgtacactggtaccagcagcttccaggcacagcccccagactcctcatctcttataacagcaatcggccctcaggggtccctgaccgattctctggctccaagtctggcacctcagcctccctggccatcactgggctccaggctgaggatgaggctgactattactgccagtcctatgacagaagcctgagtggttctgtgttcggaggaggcacccagctgaccgtcctc

SEQ ID NO: 437 - protein, 111 aa, synthetic construct
QSVLTQPPSVSGAPGQRVTISCTGSSSNIGAGYDVHWYQQLPGTAPRLLISYNSNRPSGVPDRFSGSKSGTSASLAITGLQAEDEADYYCQSYDRSLSGSVFGGGTQLTVL

SEQ ID NO: 438 - DNA, 27 bp, synthetic construct
  source 1..27: mol_type = other DNA
agctccaacatcggggcaggttatgat

SEQ ID NO: 439 - protein, 9 aa, synthetic construct
SSNIGAGYD

SEQ ID NO: 440 - DNA, 33 bp, synthetic construct
  source 1..33: mol_type = other DNA
cagtcctatgacagaagcctgagtggttctgtg

SEQ ID NO: 441 - protein, 11 aa, synthetic construct
QSYDRSLSGSV

SEQ ID NO: 442 - DNA, 1344 bp, synthetic construct
  source 1..1344: mol_type = other DNA
caggtgcagctggtggagtctgggggaggcgtggtccagcctgggaggtccctgagactctcctgtgcagcctctggattcacattcagtacctatggcatgtactgggtccgccagactccaggcaaggggctggagtgggtgacagttatatcatttgatggaaataaaaaatactatgcagactccgtgaagggccgattcaccatctccagagacaattccaaaaacacgctgtttctgcaaatgaacagcctgaaaactgaggacacggctgtatattactgtgcgaaatcttctaactggaactacggttcttttgatatatggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 443 - protein, 447 aa, synthetic construct
QVQLVESGGGVVQPGRSLRLSCAASGFTFSTYGMYWVRQTPGKGLEWVTVISFDGNKKYYADSVKGRFTISRDNSKNTLFLQMNSLKTEDTAVYYCAKSSNWNYGSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 444 - DNA, 984 bp, synthetic construct
  source 1..984: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 445 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 446 - DNA, 654 bp, synthetic construct
  source 1..654: mol_type = other DNA
cagtctgtgctgacgcagccgccctcagtgtctggggccccagggcagagggtcaccatctcctgcactgggagcagctccaacatcggggcaggttatgatgtacactggtaccagcagcttccaggcacagcccccagactcctcatctcttataacagcaatcggccctcaggggtccctgaccgattctctggctccaagtctggcacctcagcctccctggccatcactgggctccaggctgaggatgaggctgactattactgccagtcctatgacagaagcctgagtggttctgtgttcggaggaggcacccagctgaccgtcctcggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 447 - protein, 217 aa, synthetic construct
QSVLTQPPSVSGAPGQRVTISCTGSSSNIGAGYDVHWYQQLPGTAPRLLISYNSNRPSGVPDRFSGSKSGTSASLAITGLQAEDEADYYCQSYDRSLSGSVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 448 - DNA, 321 bp, synthetic construct
  source 1..321: mol_type = other DNA
ggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 449 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 450 - DNA, 375 bp, synthetic construct
  source 1..375: mol_type = other DNA
gaggtgcagttgttggagtctgggggaggcttggcacagcctggggggtccctgagactctcctgtgcagcctctggattcacctttagcagttatgccatgagctgggtccgccaggctccagggaaggggctggagtgggtctcatctgttagtggtagtggtggtaccacatattatgcagcctccgtgaagggccggttcaccgtctccagagacaattccaagaagacgctctatctgcaaatgaacagcctgagagccgaggacacggccgtatattactgtgggaaaggaggatattgtagtagtagtggttgccgtcactacggtatggacgtctggggccaagggaccacggtcaccgtctccgca

SEQ ID NO: 451 - protein, 125 aa, synthetic construct
EVQLLESGGGLAQPGGSLRLSCAASGFTFSSYAMSWVRQAPGKGLEWVSSVSGSGGTTYYAASVKGRFTVSRDNSKKTLYLQMNSLRAEDTAVYYCGKGGYCSSSGCRHYGMDVWGQGTTVTVSA

SEQ ID NO: 452 - DNA, 24 bp, synthetic construct
  source 1..24: mol_type = other DNA
ggattcacctttagcagttatgcc

SEQ ID NO: 453 - protein, 8 aa, synthetic construct
GFTFSSYA

SEQ ID NO: 454 - DNA, 24 bp, synthetic construct
  source 1..24: mol_type = other DNA
gttagtggtagtggtggtaccaca

SEQ ID NO: 455 - protein, 8 aa, synthetic construct
VSGSGGTT

SEQ ID NO: 456 - DNA, 54 bp, synthetic construct
  source 1..54: mol_type = other DNA
gggaaaggaggatattgtagtagtagtggttgccgtcactacggtatggacgtc

SEQ ID NO: 457 - protein, 18 aa, synthetic construct
GKGGYCSSSGCRHYGMDV

SEQ ID NO: 458 - DNA, 330 bp, synthetic construct
  source 1..330: mol_type = other DNA
cagtctgtgctgactcagccaccctcagcgtctgggacccccgggcagagggtcgccatttcttgttctggaagcaactccaacatcggaaataattacttatactggtaccagcagatcccaggaacgacccccaaactcctcatctatagaaataatcagcggccctcaggggtccctgaccgattctctgcctccaagtctggcacctcagcctccctggccatcagtgggctccggtccggggatgaggctgattattactgtgcagcatgggatgacagcctgagtgggtatgtcttcggaactgggaccaaggtcaccgtccta

SEQ ID NO: 459 - protein, 110 aa, synthetic construct
QSVLTQPPSASGTPGQRVAISCSGSNSNIGNNYLYWYQQIPGTTPKLLIYRNNQRPSGVPDRFSASKSGTSASLAISGLRSGDEADYYCAAWDDSLSGYVFGTGTKVTVL

SEQ ID NO: 460 - DNA, 23 bp, synthetic construct
  source 1..23: mol_type = other DNA
aactccaacatcggaaataatta

SEQ ID NO: 461 - protein, 7 aa, synthetic construct
NSNIGNN

SEQ ID NO: 462 - DNA, 33 bp, synthetic construct
  source 1..33: mol_type = other DNA
gcagcatgggatgacagcctgagtgggtatgtc

SEQ ID NO: 463 - protein, 11 aa, synthetic construct
AAWDDSLSGYV

SEQ ID NO: 464 - DNA, 1386 bp, synthetic construct
  source 1..1386: mol_type = other DNA
gaggtgcagttgttggagtctgggggaggcttggcacagcctggggggtccctgagactctcctgtgcagcctctggattcacctttagcagttatgccatgagctgggtccgccaggctccagggaaggggctggagtgggtctcatctgttagtggtagtggtggtaccacatattatgcagcctccgtgaagggccggttcaccgtctccagagacaattccaagaagacgctctatctgcaaatgaacagcctgagagccgaggacacggccgtatattactgtgggaaaggaggatattgtagtagtagtggttgccgtcactacggtatggacgtctggggccaagggaccacggtcaccgtctccgcagccaagacaacacctccttctgtgtatcctctggctcctggatgtggagatacaacaggatcttctgtgacactgggatgtctggtgaagggatattttcctgaatctgtgacagtgacatggaactctggatctctgtcttcttctgtgcatacatttcctgctctgctgcagtctggactgtatacaatgtcttcttctgtgacagtgccttcttctacatggccttctcagacagtgacatgttctgtggctcatcctgcttcttctacaacagtggataagaagctggaaccttctggacctatctctacaatcaatccttgtcctccttgtaaggaatgtcataagtgtcctgctcctaatctggaaggaggaccttctgtgtttatctttcctcctaatatcaaggatgtgctgatgatctctctgacacctaaggtgacatgtgtggtggtggatgtgtctgaagatgatcctgatgtgcagatctcttggtttgtgaataatgtggaagtgcatacagctcagacacagacacatagagaagattataattctacaatcagagtggtgtctacactgcctatccagcatcaggattggatgtctggaaaggaatttaagtgtaaggtgaataataaggatctgccttctcctatcgaaagaacaatctctaagatcaagggactggtgagagctcctcaggtgtatatcctgcctcctcctgctgaacagctgtccagaaaggatgtgtctctgacatgtctggtggtgggatttaatcctggagatatctctgtggaatggacatctaatggacatacagaagaaaattataaggatacagctcctgtgctggattctgatggatcttattttatctattctaagctgaatatgaagacatctaagtgggaaaagacagattctttttcttgtaatgtgagacatgaaggactgaagaattattatctgaagaagacaatctccagatctcctggaaagtga

SEQ ID NO: 465 - protein, 461 aa, synthetic construct
EVQLLESGGGLAQPGGSLRLSCAASGFTFSSYAMSWVRQAPGKGLEWVSSVSGSGGTTYYAASVKGRFTVSRDNSKKTLYLQMNSLRAEDTAVYYCGKGGYCSSSGCRHYGMDVWGQGTTVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 466 - DNA, 1011 bp, synthetic construct
  source 1..1011: mol_type = other DNA
gccaagacaacacctccttctgtgtatcctctggctcctggatgtggagatacaacaggatcttctgtgacactgggatgtctggtgaagggatattttcctgaatctgtgacagtgacatggaactctggatctctgtcttcttctgtgcatacatttcctgctctgctgcagtctggactgtatacaatgtcttcttctgtgacagtgccttcttctacatggccttctcagacagtgacatgttctgtggctcatcctgcttcttctacaacagtggataagaagctggaaccttctggacctatctctacaatcaatccttgtcctccttgtaaggaatgtcataagtgtcctgctcctaatctggaaggaggaccttctgtgtttatctttcctcctaatatcaaggatgtgctgatgatctctctgacacctaaggtgacatgtgtggtggtggatgtgtctgaagatgatcctgatgtgcagatctcttggtttgtgaataatgtggaagtgcatacagctcagacacagacacatagagaagattataattctacaatcagagtggtgtctacactgcctatccagcatcaggattggatgtctggaaaggaatttaagtgtaaggtgaataataaggatctgccttctcctatcgaaagaacaatctctaagatcaagggactggtgagagctcctcaggtgtatatcctgcctcctcctgctgaacagctgtccagaaaggatgtgtctctgacatgtctggtggtgggatttaatcctggagatatctctgtggaatggacatctaatggacatacagaagaaaattataaggatacagctcctgtgctggattctgatggatcttattttatctattctaagctgaatatgaagacatctaagtgggaaaagacagattctttttcttgtaatgtgagacatgaaggactgaagaattattatctgaagaagacaatctccagatctcctggaaagtga

SEQ ID NO: 467 - protein, 336 aa, synthetic construct
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 468 - DNA, 651 bp, synthetic construct
  source 1..651: mol_type = other DNA
cagtctgtgctgactcagccaccctcagcgtctgggacccccgggcagagggtcgccatttcttgttctggaagcaactccaacatcggaaataattacttatactggtaccagcagatcccaggaacgacccccaaactcctcatctatagaaataatcagcggccctcaggggtccctgaccgattctctgcctccaagtctggcacctcagcctccctggccatcagtgggctccggtccggggatgaggctgattattactgtgcagcatgggatgacagcctgagtgggtatgtcttcggaactgggaccaaggtcaccgtcctaggccaacctaaatcatctccatccgttactctcttccccccatcttcagaagaactcgaaaccaataaagccacactcgtttgcaccattacagatttctatccaggagtagttacagtcgattggaaagtagacggaacaccagttacacagggtatggaaaccacacaaccatctaagcagtctaataacaaatacatggcctcatcatacctcactcttaccgcccgcgcatgggaaagacattcatcatattcttgccaggtaacccacgaaggacacacagttgaaaaatctttgagtagagcagattgtagttga

SEQ ID NO: 469 - protein, 216 aa, synthetic construct
QSVLTQPPSASGTPGQRVAISCSGSNSNIGNNYLYWYQQIPGTTPKLLIYRNNQRPSGVPDRFSASKSGTSASLAISGLRSGDEADYYCAAWDDSLSGYVFGTGTKVTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 470 - DNA, 321 bp, synthetic construct
  source 1..321: mol_type = other DNA
ggccaacctaaatcatctccatccgttactctcttccccccatcttcagaagaactcgaaaccaataaagccacactcgtttgcaccattacagatttctatccaggagtagttacagtcgattggaaagtagacggaacaccagttacacagggtatggaaaccacacaaccatctaagcagtctaataacaaatacatggcctcatcatacctcactcttaccgcccgcgcatgggaaagacattcatcatattcttgccaggtaacccacgaaggacacacagttgaaaaatctttgagtagagcagattgtagttga

SEQ ID NO: 471 - protein, 106 aa, synthetic construct
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 472 - protein, 330 aa, synthetic construct [sequence omitted]

SEQ ID NO: 473 - DNA, 1302 bp, Homo sapiens
  source 1..1302: mol_type = genomic DNA
actcccagctcgacaaccactgccaccccccaagctcggcttgtcacctgccctaggagacgcagccgccggaccctgcccagggcacccacgcctcggcgaccaccatgtcccagaccaaaatgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcactaaccctcctgcggccctagcgaccctcaggcttcttccccaggaagcggggtcttggcctggaaccttccagagaggaggcgggagcaattttagccccaccctgctcccatctgcccccctgcaacagtcgcaggctgcttcctctctctgagttcctctgggctgccgcaggctcccctgggaatagagcaagacgtgagtcctaacctggccacagttgggggaggcagagccagcaggtggacaggtgtttgcaggggcccaacttcccctggagctcagaggtgtccccactgtaccagcctctgataagctgcctccagttgtcctttatgaacattgcagggacaacctgtgtttgccagctgggtgttccgtgtaaatagccagcctgtctctttctcggtgataaaacacaccctctctggtgagcccagcgtcccctccttggcttccaggagccctgggaagcatttttaactgggtagaatctgactgtggcttgaaataaaaagctctcagaaaa

SEQ ID NO: 474 - protein, 222 aa, Homo sapiens; identical to SEQ ID NO: 362